(19) United States
(12) Patent Application Publication
(10) Pub. No.: US 2006/0130157 A1
(43) Pub. Date: Jun. 15, 2006

(54) Ungulates with genetically modified immune systems

(76) Inventors: Kevin Wells, Christiansburg, VA (US); David Ayares, Blacksburg, VA (US)

Correspondence Address: KING & SPALDING LLP, 1180 PEACHTREE STREET, ATLANTA, GA, 30309, US

(21) Appl. No.: 11/257,817

(22) Filed: Oct. 24, 2005

Related U.S. Application Data
(60) Provisional application No. 60/621,433, filed on Oct. 22, 2004.

(51) Int. Cl. A01K 67/027

(52) U.S. Cl. 800/6; 800/15; 800/16; 800/17

(57) ABSTRACT

The present invention provides ungulate animals, tissue and organs as well as cells and cell lines derived from such animals, tissue and organs, which lack expression of functional endogenous immunoglobulin loci. The present invention also provides ungulate animals, tissue and organs as well as cells and cell lines derived from such animals, tissue and organs, which express xenogenous, such as human, immunoglobulin loci. The present invention further provides ungulate, such as porcine genomic DNA sequence of porcine heavy and light chain immunogobulins. Such animals, tissues, organs and cells can be used in research and medical therapy. In addition, methods are provided to prepare such animals, organs, tissues, and cells.

This application claims priority to U.S. provisional application No. 60/621,433 filed on Oct. 22, 2004, which is herein incorporated by reference in its entirety.

### FIELD OF THE INVENTION

The present invention provides ungulate animals, tissue and organs as well as cells and cell lines derived from such animals, tissue and organs, which lack expression of functional endogenous immunoglobulin loci. The present invention also provides ungulate animals, tissue and organs as well as cells and cell lines derived from such animals, tissue and organs, which express xenogenous, such as human, immunoglobulin loci. The present invention further provides ungulate, such as porcine genomic DNA sequence of porcine heavy and light chain immunogobulins. Such animals, tissues, organs and cells can be used in research and medical therapy. In addition, methods are provided to prepare such animals, organs, tissues, and cells.

### BACKGROUND OF THE INVENTION

An antigen is an agent or substance that can be recognized by the body as ‘foreign’. Often it is only one relatively small chemical group of a larger foreign substance which acts as the antigen, for example a component of the cell wall of a bacterium. Most antigens are proteins, though carbohydrates can act as weak antigens. Bacteria, viruses and other microorganisms commonly contain many antigens, as do pollens, dust mites, molds, foods, and other substances. The body reacts to antigens by making antibodies. Antibodies (also called immunoglobulins (Igs)) are proteins that are manufactured by cells of the immune system that bind to an antigen or foreign protein. Antibodies circulate in the serum of blood to detect foreign antigens and constitute the gamma globulin part of the blood proteins. These antibodies interact chemically with the antigen in a highly specific manner, like two pieces of a jigsaw puzzle, forming an antigen/antibody complex, or immune complex. This binding neutralises or brings about the destruction of the antigen.

When a vertebrate first encounters an antigen, it exhibits a primary humoral immune response. If the animal encounters the same antigen after a few days the immune resonse is more rapid and has a greater magnitude. The initial encounter causes specific immune cell (B-cell) clones to proliferate and differentiate. The progeny lymphocytes include not only effector cells (antibody producing cells) but also clones of memory cells, which retain the capacity to produce both effector and memory cells upon subsequent stimulation by the original antigen. The effector cells live for only a few days. The memory cells live for a lifetime and can be reactivated by a second stimuation with the same antigen. Thus, when an antigen is encountered a second time, its memory cells quickly produce effector cells which rapidly produce massive quantities of antibodies.

By exploiting the unique ability of antibodies to interact with antigens in a highly specific manner, antibodies have been developed as molecules that can be manufactured and used for both diagnostic and therapeutic applications. Because of their unique ability to bind to antigenic epitopes, polyclonal and monoclonal antibodies can be used to identify molecules carrying that epitope or can be directed, by themselves or in conjunction with another moiety, to a specific site for diagnosis or therapy. Polyclonal and monoclonal antibodies can be generated against practically any pathogen or biological target. The term polyclonal antibody refers to immune sera that usually contain pathogen-specific antibodies of various isotypes and specificities. In contrast, monoclonal antibodies consist of a single immunoglobulin type, representing one isotype with one specificity.

In 1890, Shibasaburo Kitazato and Emil Behring conducted the fundamental experiment that demonstrated immunity can be transmitted from one animal to another by transferring the serum from an immune animal to a non-immune animal. This landmark experiment laid the foundation for the introduction of passive immunization into clinical practice. However, wide scale serum therapy was largely abandoned in the 1940s because of the toxicity associated with the administration of heterologous sera and the introduction of effective antimicrobial chemotherapy. Currently, such polyclonal antibody therapy is indicated to treat infectious diseases in relatively few situations, such as replacement therapy in immunoglobulin-deficient patients, post-exposure prophylaxis against several viruses (e.g., rabies, measles, hepatitis A and B, varicella), and toxin neutralization (diphtheria, tetanus, and botulism). Despite the limited use of serum therapy, in the United States, more than 16 metric tons of human antibody are required each year for intravenous antibody therapy. Comparable levels of use exist in the economies of most highly industrialized countries, and the demand can be expected to grow rapidly in developing countries. Currently, human antibody for passive immunization is obtained from the pooled serum of donors. Thus, there is an inherent limitation in the amount of human antibody available for therapeutic and prophylactic therapies.

The use of antibodies for passive immunization against biological warfare agents represents a very promising defense strategy. The final line of defense against such agents is the immune system of the exposed individual. Current defense strategies against biological weapons include such measures as enhanced epidemiologic surveillance, vaccination, and use of antimicrobial agents. Since the potential threat of biological warfare and bioterrorism is inversely proportional to the number of immune persons in the targeted population, biological agents are potential weapons only against populations with a substantial proportion of susceptible persons.

Vaccination can reduce the susceptibility of a population against specific threats, provided that a safe vaccine exists that can induce a protective response. Unfortunately, inducing a protective response by vaccination may take longer than the time between exposure and onset of disease. Moreover, many vaccines require multiple doses to achieve a protective immune response, which would limit their usefulness in an emergency to provide rapid prophylaxis after an attack. In addition, not all vaccine recipients mount a protective response, even after receiving the recommended immunization schedule.

Drugs can provide protection when administered after exposure to certain agents, but none are available against many potential agents of biological warfare. Currently, no small-molecule drugs are available that prevent disease following exposure to preformed toxins. The only currently available intervention that could provide a state of immediate immunity is passive immunization with protective antibody (Arturo Casadevall “Passive Antibody Administration (Immediate Immunity) as a Specific Defense Against Biological Weapons” from Emerging Infectious Diseases, Posted Dec. 12, 2002).

In addition to providing protective immunity, modern antibody-based therapies constitute a potentially useful option against newly emergent pathogenic bacteria, fungi, virus and parasites (A. Casadevall and M. D. Scharff, Clinical Infectious Diseases 1995; 150). Therapies of patients with malignancies and cancer (C. Botti et al, Leukemia 1997; Suppl 2:S55-59; B. Bodey, S. E. Siegel, and H. E. Kaiser, Anticancer Res 1996; 16(2):661), therapy of steroid resistant rejection of transplanted organs as well as autoimmune diseases can also be achieved through the use of monoclonal or polyclonal antibody preparations (N. Bonnefoy-Berard and J. P. Revillard, J Heart Lung Transplant 1996; 15(5):435-442; C. Colby, et al Ann Pharmacother 1996; 30(10):1164-1174; M. J. Dugan, et al, Ann Hematol 1997; 75(1-2):41 2; W. Cendrowski, Boll Ist Sieroter Milan 1997; 58(4):339-343; L. K. Kastrukoff, et al Can J Neurol Sci 1978; 5(2):175178; J. E. Walker et al J Neurol Sci 1976; 29(2-4):303309).

Recent advances in the technology of antibody production provide the means to generate human antibody reagents, while avoiding the toxicities associated with human serum therapy. The advantages of antibody-based therapies include versatility, low toxicity, pathogen specificity, enhancement of immune function, and favorable pharmacokinetics.

The clinical use of monoclonal antibody therapeutics has just recently emerged. Monoclonal antibodies have now been approved as therapies in transplantation, cancer, infectious disease, cardiovascular disease and inflammation. In many more monoclonal antibodies are in late stage clinical trials to treat a broad range of disease indications. As a result, monoclonal antibodies represent one of the largest classes of drugs currently in development.

Despite the recent popularity of monoclonal antibodies as therapeutics, there are some obstacles for their use. For example, many therapeutic applications for monoclonal antibodies require repeated administrations, especially for chronic diseases such as autoimmunity or cancer. Because mice are convenient for immunization and recognize most human antigens as foreign, monoclonal antibodies against human targets with therapeutic potential have typically been of murine origin. However, murine monoclonal antibodies have inherent disadvantages as human therapeutics. For example, they require more frequent dosing to maintain a therapeutic level of monoclonal antibodies because of a shorter circulating half-life in humans than human antibodies. More critically, repeated administration of murine immunoglobulin creates the likelihood that the human immune system will recognize the mouse protein as foreign, generating a human anti-mouse antibody response, which can cause a severe allergic reaction. This possibility of reduced efficacy and safety has lead to the development of a number of technologies for reducing the immunogenicity of murine monoclonal antibodies.

Polyclonal antibodies are highly potent against multiple antigenic targets. They have the unique ability to target and kill a plurality of “evolving targets” linked with complex diseases. Also, of all drug classes, polyclonals have the highest probability of retaining activity in the event of antigen mutation. In addition, while monoclonals have limited therapeutic activity against infectious agents, polyclonals can both neutralize toxins and direct immune responses to eliminate pathogens, as well as biological warfare agents.

The development of polyclonal and monoclonal antibody production platforms to meet future demand for production capacity represents a promising area that is currently the subject of much research. One especially promising strategy is the introduction of human immunoglobulin genes into mice or large domestic animals. An extension of this technology would include inactivation of their endogenous immunoglobulin genes. Large animals, such as sheep, pigs and cattle, are all currently used in the production of plasma derived products, such as hyperimmune serum and clotting factors, for human use. This would support the use of human polyclonal antibodies from such species on the grounds of safety and ethics. Each of these species naturally produces considerable quantities of antibody in both serum and milk.

Arrangement of Genes Encoding Immunoglobulins

Antibody molecules are assembled from combinations of variable gene elements, and the possibilities resulting from combining the many variable gene elements in the germline enable the host to synthesize antibodies to an extraordinarily large number of antigens. Each antibody molecule consists of two classes of polypeptide chains, light (L) chains (that can be either kappa (κ) L-chain or lambda (λ) L-chain) and heavy (H) chains. The heavy and light chains join together to define a binding region for the epitope. A single antibody molecule has two identical copies of the L chain and two of the H chain. Each of the chains is comprised of a variable region (V) and a constant region (C). The variable region constitutes the antigen-binding site of the molecule. To achieve diverse antigen recognition, the DNA that encodes the variable region undergoes gene rearrangement. The constant region amino acid sequence is specific for a particular isotype of the antibody, as well as the host which produces the antibody, and thus does not undergo rearrangement.

The mechanism of DNA rearrangement is similar for the variable region of both the heavy- and light-chain loci, although only one joining event is needed to generate a light-chain gene whereas two are needed to generate a complete heavy-chain gene. The most common mode of rearrangement involves the looping-out and deletion of the DNA between two gene segments. This occurs when the coding sequences of the two gene segments are in the same orientation in the DNA. A second mode of recombination can occur between two gene segments that have opposite transcriptional orientations. This mode of recombination is less common, although such rearrangements can account for up to half of all V_{κ} to J_{κ} joins; the transcriptional orientation of half of the human V_{κ} gene segments is opposite to that of the J_{κ} gene segments.

The DNA sequence encoding a complete V region is generated by the somatic recombination of separate gene segments. The V region, or V domain, of an immunoglobulin heavy or light chain is encoded by more than one gene segment. For the light chain, the V domain is encoded by two separate DNA segments. The first segment encodes the first 95-101 amino acids of the light chain and is termed a V gene segment because it encodes most of the V domain. The second segment encodes the remainder of the V domain (up to 13 amino acids) and is termed a joining or J gene segment. The joining of a V and a J gene segment creates a continuous exon that encodes the whole of the light-chain V region. To make a complete immunoglobulin light-chain messenger RNA, the V-region exon is joined to the C-region sequence by RNA splicing after transcription.

A heavy-chain V region is encoded in three gene segments. In addition to the V and J gene segments (denoted V_{H} and J_{H} to distinguish them from the light-chain V_{L} and J_{L}), there is a third gene segment called the diversity or D_{H} gene segment, which lies between the V_{H} and J_{H} gene segments. The process of recombination that generates a complete heavy-chain V region occurs in two separate stages. In the first, a D_{H} gene segment is joined to a J_{H} gene segment; then a V_{H} gene segment rearranges to DJ_{H} to make a complete V_{H}-region exon. As with the light-chain genes, RNA splicing joins the assembled V-region sequence to the neighboring C-region gene.

Diversification of the antibody repertoire occurs in two stages: primarily by rearrangement (“V(D)J recombination”) of Ig V, D and J gene segments in precursor B cells resident in the bone marrow, and then by somatic mutation and class switch recombination of these rearranged Ig genes when mature B cells are activated. Immunoglobulin somatic mutation and class switching are central to the maturation of the immune response and the generation of a “memory” response.

The genomic loci of antibodies are very large and they are located on different chromosomes. The immunoglobulin gene segments are organized into three clusters or genetic loci: the κ, λ, and heavy-chain loci. Each is organized slightly differently. For example, in humans, immunoglobulin genes are organized as follows. The λ light-chain locus is located on chromosome 22 and a cluster of V_{λ} gene segments is followed by four sets of J_{λ} gene segments each linked to a single C_{λ} gene. The κ light-chain locus is on chromosome 2 and the cluster of V_{κ} gene segments is followed by a cluster of J_{λ} gene segments, and then by a single C_{λ} gene. The organization of the heavy-chain locus, on chromosome 14, resembles that of the κ locus, with separate clusters of V_{H}, D_{H}, and J_{H} gene segments and of C_{H} genes. The heavy-chain locus differs in one important way: instead of a single C-region, it contains a series of C regions arrayed one after the other, each of which corresponds to a different isotype. There are five immunoglobulin heavy chain isotypes: IgM, IgG, IgA, IgE and IgD. Generally, a cell expresses only one at a time, beginning with IgM. The expression of other isotypes, such as IgG, can occur through isotype switching.

The joining of various V, D and J genes is an entirely random event that results in approximately 50,000 different possible combinations for VDJ(H) and approximately 1,000 for VJ(L). Subsequent random pairing of H and L chains brings the total number of antibody specificities to about 10⁷ possibilities. Diversity is further increased by the imprecise joining of different genetic segments. Rearrangements occur on both DNA strands, but only one strand is transcribed (due to allelic exclusion). Only one rearrangement occurs in the life of a B cell because of irreversible deletions in DNA. Consequently, each mature B cell maintains one immunologic specificity and is maintained in the progeny or clone. This constitutes the molecular basis of the clonal selection; i.e., each antigenic determinant triggers the response of the pre-existing clone of B lymphocytes bearing the specific receptor molecule. The primary repertoire of B cells, which is established by V(D)J recombination, is primarily controlled by two closely linked genes, recombination activating gene (RAG)-1 and RAG-2.

Over the last decade, considerable diversity among vertebrates in both Ig gene diversity and antibody repertoire development has been revealed. Rodents and humans have five heavy chain classes, IgM, IgD, IgG, IgE and IgA, and each have four subclasses of IgG and one or two subclasses of IgA, while rabbits have a single IgG heavy chain gene but 13 genes for different IgA subclasses (Burnett, R. C et al. EMBO J 8:4047; Honjo, In Honjo, T, Alt. F. W. T. H. eds, Immunoglobulin Genes p. 123 Academic Press, New York). Swine have at least six IgG subclasses (Kacskovics, I et al. 1994 J Immunol 153:3565), but no IgD (Butler et al. 1996 Inter. Immunol 8:1897-1904). A gene encoding IgD has only been described in rodents and primates. Diversity in the mechanism of repertoire development is exemplified by contrasting the pattern seen in rodents and primates with that reported for chickens, rabbits, swine and the domesticated Bovidae. Whereas the former group have a large number of V_{H} genes belonging to seven to 10 families (Rathbun, G. In Hongo, T. Alt. F. W. and Rabbitts, T. H., eds, Immunoglobulin Genes, p. 63, Academic press New York), the V_{H} genes of each member of the latter group belong to a single V_{H} gene family (Sun, J. et al. 1994 J. Immunol. 1553:56118; Dufour, V et al.1996, J Immunol. 156:2163). With the exception of the rabbit, this family is composed of less than 25 genes. Whereas rodents and primates can utilize four to six J_{H} segments, only a single J_{H} is available for repertoire development in the chicken (Reynaud et al. 1989 Adv. Immunol. 57:353). Similarly, Butler et al. (1996 Inter. Immunol 8:1897-1904) hypothesized that swine may resemble the chicken in having only a single J_{H} gene. These species generally have fewer V, D and J genes; in the pig and cow a single VH gene family exists, consisting of less than 20 gene segments (Butler et al, Advances in Swine in Biomedical Research, eds: Tumbleson and Schook, 1996; Sinclair et al, J. Immunol. 159: 3883, 1997). Together with lower numbers of J and D gene. segments, this results in significantly less diversity being generated by gene rearrangement. However, there does appear to be greater numbers of light chain genes in these species. Similar to humans and mice, these species express a single K light chain but multiple λ light chain genes. However, these do not seem to affect the restricted diversity that is achieved by rearrangement.

Since combinatorial joining of more than 100 V_{H}, 20-30 D_{H} and four to six J_{H} gene segments is a major mechanism of generating the antibody repertoire in humans, species with fewer V_{H}, D_{H} or J_{H} segments must either generate a smaller repertoire or use alternative mechanisms for repertoire development. Ruminants, pigs, rabbits and chickens, utilize several mechanisms to generate antibody diversity. In these species there appears to be an important secondary repertoire development, which occurs in highly specialized lymphoid tissue such as ileal Peyer's patches (Binns and Licence, Adv. Exp. Med. Biol. 186: 661, 1985). Secondary repertoire development occurs in these species by a process of somatic mutation which is a random and not fully understood process. The mechanism for this repertoire diversification appears to be templated mutation, or gene conversion (Sun et al, J. Immunol. 153: 5618, 1994) and somatic hypermutation.

Gene conversion is important for antibody diversification in some higher vertebrates, such as chickens, rabbits and cows. In mice, however, conversion events appear to be infrequent among endogenous antibody genes. Gene conversion is a distinct diversifying mechanism characterized by transfers of homologous sequences from a donor antibody V gene segment to an acceptor V gene segment. If donor and acceptor segments have numerous sequence differences then gene conversion can introduce a set of sequence changes into a V region by a single event. Depending on the species, gene conversion events can occur before and/or after antigen exposure during B cell differentiation (Tsai et al. International Immunology, Vol. 14, No. 1, 55-64, January 2002).

Somatic hypermutation achieves diversification of antibody genes in all higher vertebrate species. It is typified by the introduction of single point mutations into antibody V(D)J segments. Generally, hypermutation appears to be activated in B cells by antigenic stimulation.

Production of Animals with Humanized Immune Systems

In order to reduce the immunogenicity of antibodies generated in mice for human therapeutics, various attempts have been made to replace murine protein sequences with human protein sequences in a process now known as humanization. Transgenic mice have been constructed which have had their own immunoglobulin genes functionally replaced with human immunoglobulin genes so that they produce human antibodies upon immunization. Elimination of mouse antibody production was achieved by inactivation of mouse Ig genes in embryonic stem (ES) cells by using gene-targeting technology to delete crucial cis-acting sequences involved in the process of mouse Ig gene rearrangement and expression. B cell development in these mutant mice could be restored by the introduction of megabase-sized YACs containing a human germline-configuration H- and κ L-chain minilocus transgene. The expression of fully human antibody in these transgenic mice was predominant, at a level of several 100 μg/l of blood. This level of expression is several hundred-fold higher than that detected in wild-type mice expressing the human Ig gene, indicating the importance of inactivating the endogenous mouse Ig genes in order to enhance human antibody production by mice.

The first humanization attempts utilized molecular biology techniques to construct recombinant antibodies. For example, the complementarity determining regions (CDR) from a mouse antibody specific for a hapten were grafted onto a human antibody framework, effecting a CDR replacement. The new antibody retained the binding specificity conveyed by the CDR sequences (P. T. Jones et al. Nature 321: 522-525 (1986)). The next level of humanization involved combining an entire mouse VH region with a human constant region such as gamma₁ (S. L. Morrison et al., Proc. Natl. Acad. Sci., 81, pp. 6851-6855 (1984)). However, these chimeric antibodies, which still contain greater than 30% xenogeneic sequences, are sometimes only marginally less immunogenic than totally xenogeneic antibodies (M. Bruggemarm et al., J. Exp. Med., 170, pp. 2153-2157 (1989)).

Subsequently, attempts were carried out to introduce human immunoglobulin genes into the mouse, thus creating transgenic mice capable of responding to antigens with antibodies having human sequences (Bruggemann et al. Proc. Nat'l. Acad. Sci. USA 86:6709-6713 (1989)). Due to the large size of human immunoglobulin genomic loci, these attempts were thought to be limited by the amount of DNA, which could be stably maintained by available cloning vehicles. As a result, many investigators concentrated on producing mini-loci containing limited numbers of V region genes and having altered spatial distances between genes as compared to the natural or germline configuration (See, for example, U.S. Pat. No. 5,569,825). These studies indicated that producing human sequence antibodies in mice was possible, but serious obstacles remained regarding obtaining sufficient diversity of binding specificities and effector functions (isotypes) from these transgenic animals to meet the growing demand for antibody therapeutics.

In order to provide additional diversity, work has been conducted to add large germline fragments of the human Ig locus into transgenic mammals. For example, a majority of the human V, D, and J region genes arranged with the same spacing found in the unrearranged germline of the human genome and the human Cμ and Cδ constant regions was introduced into mice using yeast artificial chromosome (YAC) cloning vectors (See, for example, WO 94/02602). A 22 kb DNA fragment comprising sequences encoding a human gamma-2 constant region and the upstream sequences required for class-switch recombination was latter appended to the foregoing transgene. In addition, a portion of a human kappa locus comprising Vλ, Jλ and Cλ region genes, also arranged with substantially the same spacing found in the unrearranged germline of the human genome, was introduced into mice using YACS. Gene targeting was used to inactivate the murine IgH & kappa light chain immunoglobulin gene loci and such knockout strains were bred with the above transgenic strains to generate a line of mice having the human V, D, J, Cμ, Cδ. and Cγ₂ constant regions as well as the human Vκ, Jκ and Cκ region genes all on an inactivated murine immunoglobulin background (See, for example, PCT patent application WO 94/02602 to Kucherlapati et al.; see also Mendez et al., Nature Genetics 15:146-156 (1997)).

Yeast artificial chromosomes as cloning vectors in combination with gene targeting of endogenous loci and breeding of transgenic mouse strains provided one solution to the problem of antibody diversity. Several advantages were obtained by this approach. One advantage was that YACs can be used to transfer hundreds of kilobases of DNA into a host cell. Therefore, use of YAC cloning vehicles allows inclusion of substantial portions of the entire human Ig heavy and light chain regions into a transgenic mouse thus approaching the level of potential diversity available in the human. Another advantage of this approach is that the large number of V genes has been shown to restore full B cell development in mice deficient in murine immunoglobulin production. This ensures that these reconstituted mice are provided with the requisite cells for mounting a robust human antibody response to any given immunogen. (See, for example, WO 94/02602.; L. Green and A. Jakobovits, J. Exp. Med. 188:483-495 (1998)). A further advantage is that sequences can be deleted or inserted onto the YAC by utilizing high frequency homologous recombination in yeast. This provides for facile engineering of the YAC transgenes.

In addition, Green et al. Nature Genetics 7:13-21 (1994) describe the generation of YACs containing 245 kb and 190 kb-sized germline configuration fragments of the human heavy chain locus and kappa light chain locus, respectively, which contained core variable and constant region sequences. The work of Green et al. was recently extended to the introduction of greater than approximately 80% of the human antibody repertoire through introduction of megabase sized, germline configuration YAC fragments of the human heavy chain loci and kappa light chain loci, respectively, to produce XenoMouse™ mice. See, for example, Mendez et al. Nature Genetics 15:146-156 (1997), Green and Jakobovits J. Exp. Med. 188:483-495 (1998), European Patent No. EP 0 463 151 B1, PCT Publication Nos. WO 94/02602, WO 96/34096 and WO 98/24893.

Several strategies exist for the generation of mammals that produce human antibodies. In particular, there is the “minilocus” approach that is typified by work of GenPharm International, Inc. and the Medical Research Council, YAC introduction of large and substantially germline fragments of the Ig loci that is typified by work of Abgenix, Inc. (formerly Cell Genesys). The introduction of entire or substantially entire loci through the use microcell fusion as typified by work of Kirin Beer Kabushiki Kaisha.

In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (such as a gamma constant region) are formed into a construct for insertion into an animal. See, for example, U.S. Pat. Nos. 5,545,807, 5,545,806, 5,625,825, 5,625,126, 5,633,425, 5,661,016, 5,770,429, 5,789,650, 5,814,318, 5,591,669, 5,612,205, 5,721,367, 5,789,215, 5,643,763; European Patent No. 0 546 073; PCT Publication Nos. WO 92/03918, WO 92/22645, WO 92/22647, WO 92/22670, WO 93/12227, WO 94/00569, WO 94/25585, WO 96/14436, WO 97/13852, and WO 98/24884; Taylor et al. Nucleic Acids Research 20:6287-6295 (1992), Chen et al. International Immunology 5:647-656 (1993), Tuaillon et al. J. Immunol. 154:6453-6465 (1995), Choi et al. Nature Genetics 4:117-123 (1993), Lonberg et al. Nature 368:856-859 (1994), Taylor et al. International Immunology 6:579-591 (1994), Tuaillon et al. J. Immunol. 154:6453-6465 (1995), and Fishwild et al. Nature Biotech. 14:845-851 (1996).

In the microcell fusion approach, portions or whole human chromosomes can be introduced into mice (see, for example, European Patent Application No. EP 0 843 961 A1). Mice generated using this approach and containing the human Ig heavy chain locus will generally possess more than one, and potentially all, of the human constant region genes. Such mice will produce, therefore, antibodies that bind to particular antigens having a number of different constant regions.

While mice remain the most developed animal for the expression of human immunoglobulins in humans, recent technological advances have allowed for progress to begin in applying these techniques to other animals, such as cows. The general approach in mice has been to genetically modify embryonic stem cells of mice to knock-out murine immunoglobulins and then insert YACs containing human immunoglobulins into the ES cells. However, ES cells are not available for cows or other large animals such as sheep and pigs. Thus, several fundamental developments had to occur before even the possibility existed to generate large animals with immunoglobulin genes knocked-out and that express human antibody. The alternative to ES cell manipulation to create genetically modified animals is cloning using somatic cells that have been genetically modified. Cloning using genetically modified somatic cells for nuclear transfer has only recently been accomplished.

Since the announcement of Dolly's (a cloned sheep) birth from an adult somatic cell in 1997 (Wilmut, I., et al (1997) Nature 385: 810-813), ungulates, including cattle (Cibelli, J et al 1998 Science 280: 1266-1258; Kubota, C. et al.2000 Proc. Nat'l. Acad. Sci 97: 990-995), goats (Baguisi, A. et al., (1999) Nat. Biotechnology 17: 456-461), and pigs (Polejaeva, I. A., et al. 2000 Nature 407: 86-90; Betthauser, J. et al. 2000 Nat. Biotechnology 18: 1055-1059) have been cloned.

The next technological advance was the development of the technique to genetically modify the cells prior to nuclear transfer to produce genetically modified animals. PCT publication No. WO 00/51424 to PPL Therapeutics describes the targetted genetic modification of somatic cells for nuclear transfer.

Subsequent to these fundamental developments, single and double allele knockouts of genes and the birth of live animals with these modifications have been reported. Between 2002 and 2004, three independent groups, Immerge Biotherapeutics, Inc. in collaboration with the University of Missouri (Lai et al. (Science (2002) 295: 1089-1092) & Kolber-Simonds et al. (PNAS. (2004) 101(19):7335-40)), Alexion Pharmaceuticals (Ramsoondar et al. (Biol Reprod (2003)69: 437-445) and Revivicor, Inc. (Dai et al. (Nature Biotechnology (2002) 20: 251-255) & Phelps et al. (Science (2003) Jan 17;299(5605):411-4)) produced pigs that lacked one allele or both alleles of the alpha-1,3-GT gene via nuclear transfer from somatic cells with targeted genetic deletions. In 2003, Sedai et al. (Transplantation (2003) 76:900-902) reported the targeted disruption of one allele of the alpha-1,3-GT gene in cattle, followed by the successful nuclear transfer of the nucleus of the genetically modified cell and production of transgenic fetuses.

Thus, the feasibility of knocking-out immunoglobulin genes in large animals and inserting human immunoglobulin loci into their cells is just now beginning to be explored. However, due to the complexity and species differences of immunoglobulin genes, the genomic sequences and arrangement of Ig kappa, lambda and heavy chains remain poorly understood in most species. For example, in pigs, partial genomic sequence and organization has only been described for heavy chain constant alpha, heavy chain constant mu and heavy chain constant delta (Brown and Butler Mol Immunol. June 1994;31(8):633-42, Butler et al Vet Immunol Immunopathol. October 1994;43(1-3):5-12, and Zhao et al J Immunol. Aug. 1, 2003;171(3):1312-8).

In cows, the immunoglobulin heavy chain locus has been mapped (Zhao et al. 2003 J. Biol. Chem. 278:35024-32) and the cDNA sequence for the bovine kappa gene is known (See, for example, U.S. Patent Publication No. 2003/0037347). Further, approximately 4.6 kb of the bovine mu heavy chain locus has been sequenced and transgenic calves with decreased expression of heavy chain immunoglobulins have been created by disrupting one or both alleles of the bovine mu heavy chain. In addition, a mammalian artificial chromosome (MAC) vector containing the entire unarranged sequences of the human Ig H-chain and κ L-chain has been introduced into cows (TC cows) with the technology of microcell-mediated chromosome transfer and nuclear transfer of bovine fetal fibroblast cells (see, for example, Kuroiwa et al. 2002 Nature Biotechnology 20:889, Kuroiwa et al. 2004 Nat Genet. June 6 Epub, U.S. Patent Publication Nos. 2003/0037347, 2003/0056237, 2004/0068760 and PCT Publication No. WO 02/07648).

While significant progress has been made in the production of bovine that express human immunoglobulin, little has been accomplished in other large animals, such as sheep, goats and pigs. Although cDNA sequence information for immunoglobulin genes of sheeps, goats and pigs is readily available in Genbank, the unique nature of immunoglobulin loci, which undergo massive rearrangements, creates the need to characterize beyond sequences known to be present in mRNAs (or cDNAs). Since immunoglobulin loci are modular and the coding regions are redundant, deletion of a known coding region does not ensure altered function of the locus. For example, if one were to delete the coding region of a heavy-chain variable region, the function of the locus would not be significantly altered because hundreds of other function variable genes remain in the locus. Therefore, one must first characterize the locus to identify a potential “Achilles heel”.

Despite some advancements in expressing human antibodies in cattle, greater challenges remain for inactivation of the endogenous bovine Ig genes, increasing expression levels of the human antibodies and creating human antibody expression in other large animals, such as porcine, for which the sequence and arrangement of immunoglobulin genes are largely unknown.

It is therefore an object of the present invention to provide the arrangement of ungulate immunoglobin germline gene sequence.

It is another object of the presenst invention to provide novel ungulate immunoglobulin genomic sequences.

It is a further object of the present invention to provide cells, tissues and animals lacking at least one allele of a heavy and/or light chain immunoglobulin gene.

It is another object of the present invention to provide ungulates that express human immunoglobulins.

It is a still further object of the present invention to provide methods to generate cells, tissues and animals lacking at least one allele of novel ungulate immunoglobulin gene sequences and/or express human immunoglobulins.

### SUMMARY OF THE INVENTION

The present invention provides for the first time ungulate immunoglobin germline gene sequence arrangement as well as novel genomic sequences thereof. In addition, novel ungulate cells, tissues and animals that lack at least one allele of a heavy or light chain immunoglobulin gene are provided. Based on this discovery, ungulates can be produced that completely lack at least one allele of a heavy and/or light chain immunoglobulin gene. In addition, these ungulates can be further modified to express xenoogenous, such as human, immunoglobulin loci or fragments thereof.

In one aspect of the present invention, a transgenic ungulate that lacks any expression of functional endogenous immunoglobulins is provided. In one embodiment, the ungulate can lack any expression of endogenous heavy and/or light chain immunoglobulins. The light chain immunoglobulin can be a kappa and/or lambda immunoglobulin. In additional embodiments, transgenic ungulates are provided that lack expression of at least one allele of an endogenous immunoglobulin wherein the immunoglobulin is selected from the group consisting of heavy chain, kappa light chain and lambda light chain or any combination thereof. In one embodiment, the expression of functional endogenous immunoglobulins can be accomplished by genetic targeting of the endogenous immunoglobulin loci to prevent expression of the endogenous immunoglobulin. In one embodiment, the genetic targeting can be accomplished via homologous recombination. In another embodiment, the transgenic ungulate can be produced via nuclear transfer.

In other embodiments, the transgenic ungulate that lacks any expression of functional endogenous immunoglobulins can be further genetically modified to express an xenogenous immunoglobulin loci. In an alternative embodiment, porcine animals are provided that contain an xenogeous immunoglobulin locus. In one embodiment, the xenogeous immunoglobulin loci can be a heavy and/or light chain immunoglobulin or fragment thereof. In another embodiment, the xenogenous immunoglobulin loci can be a kappa chain locus or fragment thereof and/or a lambda chain locus or fragment thereof. In still further embodiments, an artificial chromosome (AC) can contain the xenogenous immunoglobulin. In one embodiment, the AC can be a yeast AC or a mammalian AC. In a further embodiment, the xenogenous locus can be a human immunoglobulin locus or fragment thereof. In one embodiment, the human immunoglobulin locus can be human chromosome 14, human chromosome 2, and human chromosome 22 or fragments thereof. In another embodiment, the human immunoglobulin locus can include any fragment of a human immunoglobulin that can undergo rearrangement. In a further embodiment, the human immunoglobulin loci can include any fragment of a human immunoglobulin heavy chain and a human immunoglobulin light chain that can undergo rearrangement. In still further embodiment, the human immunoglobulin loci can include any human immunoglobulin locus or fragment thereof that can produce an antibody upon exposure to an antigen. In a particular embodiment, the exogenous human immunoglobulin can be expressed in B cells to produce xenogenous immunoglobulin in response to exposure to one or more antigens.

In another aspect of the present invention, transgenic ungulates are provided that expresses a xenogenous immunoglobulin loci or fragment thereof, wherein the immunoglobulin can be expressed from an immunoglobulin locus that is integrated within an endogenous ungulate chromosome. In one embodiment, ungulate cells derived from the transgenic animals are provided. In one embodiment, the xenogenous immunoglobulin locus can be inherited by offspring. In another embodiment, the xenogenous immunoglobulin locus can be inherited through the male germ line by offspring. In still further embodiments, an artificial chromosome (AC) can contain the xenogenous immunoglobulin. In one embodiment, the AC can be a yeast AC or a mammalian AC. In a further embodiment, the xenogenous locus can be a human immunoglobulin locus or fragment thereof. In one embodiment, the human immunoglobulin locus can be human chromosome 14, human chromosome 2, and human chromosome 22 or fragments thereof. In another embodiment, the human immunoglobulin locus can include any fragment of a human immunoglobulin that can undergo rearrangement. In a further embodiment, the human immunoglobulin loci can include any fragment of a human immunoglobulin heavy chain and a human immunoglobulin light chain that can undergo rearrangement. In still further embodiment, the human immunoglobulin loci can include any human immunoglobulin locus or fragment thereof that can produce an antibody upon exposure to an antigen. In a particular embodiment, the exogenous human immunoglobulin can be expressed in B cells to produce xenogenous immunoglobulin in response to exposure to one or more antigens.

In another aspect of the present invention, novel genomic sequences encoding the heavy chain locus of ungulate immunoglobulin are provided. In one embodiment, an isolated nucleotide sequence encoding porcine heavy chain is provided that includes at least one variable region, two diversity regions, at least four joining regions and at least one constant region, such as the mu constant region, for example, as represented in Seq ID No. 29. In another embodiment, an isolated nucleotide sequence is provided that includes at least four joining regions and at least one constant region, such as as the mu constant region, of the porcine heavy chain genomic sequence, for example, as represented in Seq ID No. 4. In a further embodiment, nucleotide sequence is provided that includes 5′ flanking sequence to the first joining region of the porcine heavy chain genomic sequence, for example, as represented in Seq ID No 1. Still further, nucleotide sequence is provided that includes 3′ flanking sequence to the first joining region of the porcine heavy chain genomic sequence, for example, as represented in the 3′ region of Seq ID No 4. In further embodiments, isolated nucleotide sequences as depicted in Seq ID Nos 1, 4 or 29 are provided. Nucleic acid sequences at least 80, 85, 90, 95, 98 or 99% homologous to Seq ID Nos 1, 4 or 29 are also provided. In addition, nucleotide sequences that contain at least 10, 15, 17, 20, 25 or 30 contiguous nucleotides of Seq ID Nos 1, 4 or 29 are provided. In one embodiment, the nucleotide sequence contains at least 17, 20, 25 or 30 contiguous nucleotides of Seq ID No 4 or residues 1-9,070 of Seq ID No 29.

In another embodiment, the nucleotide sequence contains residues 9,070-11039 of Seq ID No 29. Further provided are nucleotide sequences that hybridizes, optionally under stringent conditions, to Seq ID Nos 1, 4 or 29, as well as, nucleotides homologous thereto.

In another embodiment, novel genomic sequences encoding the kappa light chain locus of ungulate immunoglobulin are provided. The present invention provides the first reported genomic sequence of ungulate kappa light chain regions. In one embodiment, nucleic acid sequence is provided that encodes the porcine kappa light chain locus. In another embodiment, the nucleic acid sequence can contain at least one joining region, one constant region and/or one enhancer region of kappa light chain. In a further embodiment, the nucleotide sequence can include at least five joining regions, one constant region and one enhancer region, for example, as represented in Seq ID No. 30. In a further embodiment, an isolated nucleotide sequence is provided that contains at least one, at least two, at least three, at least four or five joining regions and 3′ flanking sequence to the joining region of porcine genomic kappa light chain, for example, as represented in Seq ID No 12. In another embodiment, an isolated nucleotide sequence of porcine genomic kappa light chain is provided that contains 5′ flanking sequence to the first joining region, for example, as represented in Seq ID No 25. In a further embodiment, an isolated nucleotide sequence is provided that contains 3′ flanking sequence to the constant region and, optionally, the 5′ portion of the enhancer region, of porcine genomic kappa light chain, for example, as represented in Seq ID Nos. 15, 16 and/or 19.

In further embodiments, isolated nucleotide sequences as depicted in Seq ID Nos 30, 12, 25, 15, 16 or 19 are provided. Nucleic acid sequences at least 80, 85, 90, 95, 98 or 99% homologous to Seq ID Nos 30, 12, 25, 15, 16 or 19 are also provided. In addition, nucleotide sequences that contain at least 10, 15, 17, 20, 25 or 30 contiguous nucleotides of Seq ID Nos 30, 12, 25, 15, 16 or 19 are provided. Further provided are nucleotide sequences that hybridizes, optionally under stringent conditions, to Seq ID Nos 30, 12, 25, 15, 16 or 19, as well as, nucleotides homologous thereto.

In another embodiment, novel genomic sequences encoding the lambda light chain locus of ungulate immunoglobulin are provided. The present invention provides the first reported genomic sequence of ungulate lambda light chain regions. In one embodiment, the porcine lambda light chain nucleotides include a concatamer of J to C units. In a specific embodiment, an isolated porcine lambda nucleotide sequence is provided, such as that depicted in Seq ID No. 28. In one embodiment, nucleotide sequence is provided that includes 5′ flanking sequence to the first lambda J/C region of the porcine lambda light chain genomic sequence, for example, as represented by Seq ID No 32. Still -further, nucleotide sequence is provided that includes 3′ flanking sequence to the J/C cluster region of the porcine lambda light chain genomic sequence, for example, approximately 200 base pairs downstream of lambda J/C, such as that represented by Seq ID No 33. Alternatively, nucleotide sequence is provided that includes 3′ flanking sequence to the J/C cluster region of the porcine lambda light chain genomic sequence, for example, as represented by Seq ID No 34, 35, 36, 37, 38, and/or 39. In a further embodiment, nucleic acid sequences are provided that encode bovine lambda light chain locus, which can include at least one joining region-constant region pair and/or at least one variable region, for example, as represented by Seq ID No. 31. In further embodiments, isolated nucleotide sequences as depicted in Seq ID Nos 28, 31, 32, 33, 34, 35, 36, 37, 38, or 39 are provided. Nucleic acid sequences at least 80, 85, 90, 95, 98 or 99% homologous to Seq ID Nos 28, 31, 32, 33, 34, 35, 36, 37, 38, or 39 are also provided. In addition, nucleotide sequences that contain at least 10, 15, 17, 20, 25 or 30 contiguous nucleotides of Seq ID Nos 28, 31, 32, 33, 34, 35, 36, 37, 38, or 39 are provided. Further provided are nucleotide sequences that hybridizes, optionally under stringent conditions, to Seq ID Nos 28, 31, 32, 33, 34, 35, 36, 37, 38, or 39, as well as, nucleotides homologous thereto.

In another embodiment, nucleic acid targeting vector constructs are also provided. The targeting vectors can be designed to accomplish homologous recombination in cells. These targeting vectors can be transformed into mammalian cells to target the ungulate heavy chain, kappa light chain or lambda light chain genes via homologous recombination. In one embodiment, the targeting vectors can contain a 3′ recombination arm and a 5′ recombination arm (i.e. flanking sequence) that is homologous to the genomic sequence of ungulate heavy chain, kappa light chain or lambda light chain genomic sequence, for example, sequence represented by Seq ID Nos. 1, 4, 29, 30, 12, 25, 15, 16, 19, 28 or 31, as described above. The homologous DNA sequence can include at least 15 bp, 20 bp, 25 bp, 50 bp, 100 bp, 500 bp, 1 kbp, 2 kbp, 4 kbp, 5 kbp, 10 kbp, 15 kbp, 20 kbp, or 50 kbp of sequence homologous to the genomic sequence. The 3′ and 5′ recombination arms can be designed such that they flank the 3′ and 5′ ends of at least one functional variable, joining, diversity, and/or constant region of the genomic sequence. The targeting of a functional region can render it inactive, which results in the inability of the cell to produce functional immunoglobulin molecules. In another embodiment, the homologous DNA sequence can include one or more intron and/or exon sequences. In addition to the nucleic acid sequences, the expression vector can contain selectable marker sequences, such as, for example, enhanced Green Fluorescent Protein (eGFP) gene sequences, initiation and/or enhancer sequences, poly A-tail sequences, and/or nucleic acid sequences that provide for the expression of the construct in prokaryotic and/or eukaryotic host cells. The selectable marker can be located between the 5′ and 3′ recombination arm sequence.

In one particular embodiment, the targeting vector can contain 5′ and 3′ recombination arms that contain homologous sequence to the 3′ and 5′ flanking sequence of the J6 region of the porcine immunoglobulin heavy chain locus. Since the J6 region is the only functional joining region of the porcine immunoglobulin heavy chain locus, this will prevent the exression of a functional porcine heavy chain immunoglobulin. In a specific embodiment, the targeting vector can contain a 5′ recombination arm that contains sequence homologous to genomic sequence 5′ of the J6 region, including J1-4, and a 3′ recombination arm that contains sequence homologous to genomic sequence 3′ of the J6 region, including the mu constant region (a “J6 targeting construct”), see for example, FIG. 1. Further, this J6 targeting construct can also contain a selectable marker gene that is located between the 5′ and 3′ recombination arms, see for example, Seq ID No 5 and FIG. 1. In other embodiments, the targeting vector can contain a 5′ recombination arm that contains sequence homologous to genomic sequence 5′ of the diversity region, and a 3′ recombination arm that contains sequence homologous to genomic sequence 3′ of the diversity region of the porcine heavy chain locus. In a further embodiment, the targeting vector can contain a 5′ recombination arm that contains sequence homologous to genomic sequence 5′ of the mu constant region and a 3′ recombination arm that contains sequence homologous to genomic sequence 3′ of the mu constant region of the porcine heavy chain locus.

In another particular embodiment, the targeting vector can contain 5′ and 3′ recombination arms that contain homologous sequence to the 3′ and 5′ flanking sequence of the constant region of the porcine immunoglobulin heavy chain locus. Since the present invention discovered that there is only one constant region of the porcine immunoglobulin kappa light chain locus, this will prevent the expression of a functional porcine kappa light chain immunoglobulin. In a specific embodiment, the targeting vector can contain a 5′ recombination arm that contains sequence homologous to genomic sequence 5′ of the constant region, optionally including the joining region, and a 3′ recombination arm that contains sequence homologous to genomic sequence 3′ of the constant region, optionally including at least part of the enhancer region (a “Kappa constant targeting construct”), see for example, FIG. 2. Further, this kappa constant targeting construct can also contain a selectable marker gene that is located between the 5′ and 3′ recombination arms, see for example, Seq ID No 20 and FIG. 2. In other embodiments, the targeting vector can contain a 5′ recombination arm that contains sequence homologous to genomic sequence 5′ of the joining region, and a 3′ recombination arm that contains sequence homologous to genomic sequence 3′ of the joining region of the porcine kappa light chain locus.

In another embodiment, primers are provided to generate 3′ and 5′ sequences of a targeting vector. The oligonucleotide primers can be capable of hybridizing to porcine immunoglobulin genomic sequence, such as Seq ID Nos. 1, 4, 29, 30, 12, 25, 15, 16, 19, 28 or 31, as described above. In a particular embodiment, the primers hybridize under stringent conditions to Seq ID Nos. 1, 4, 29, 30, 12, 25, 15, 16, 19, 28 or 31, as described above. Another embodiment provides oligonucleotide probes capable of hybridizing to porcine heavy chain, kappa light chain or lambda light chain nucleic acid sequences, such as Seq ID Nos. 1, 4, 29, 30, 12, 25, 15, 16, 19, 28 or 31, as described above. The polynucleotide primers or probes can have at least 14 bases, 20 bases, 30 bases, or 50 bases which hybridize to a polynucleotide of the present invention. The probe or primer can be at least 14 nucleotides in length, and in a particular embodiment, are at least 15, 20, 25, 28, or 30 nucleotides in length.

In one embodiment, primers are provided to amplify a fragment of porcine Ig heavy-chain that includes the functional joining region (the J6 region). In one non-limiting embodiment, the amplified fragment of heavy chain can be represented by Seq ID No 4 and the primers used to amplify this fragment can be complementary to a portion of the J-region, such as, but not limited to Seq ID No 2, to produce the 5′ recombination arm and complementary to a portion of Ig heavy-chain mu constant region, such as, but not limited to Seq ID No 3, to produce the 3′ recombination arm. In another embodiment, regions of the porcine Ig heavy chain (such as, but not limited to Seq ID No 4) can be subcloned and assembled into a targeting vector.

In other embodiments, primers are provided to amplify a fragment of porcine Ig kappa light-chain that includes the constant region. In another embodiment, primers are provided to amplify a fragment of porcine Ig kappa light-chain that includes the J region. In one non-limiting embodiment, the primers used to amplify this fragment can be complementary to a portion of the J-region, such as, but not limited to Seq ID No 21 or 10, to produce the 5′ recombination arm and complementary to genomic sequence 3′ of the constant region, such as, but not limited to Seq ID No 14, 24 or 18, to produce the 3′ recombination arm. In another embodiment, regions of the porcine Ig heavy chain (such as, but not limited to Seq ID No 20) can be subcloned and assembled into a targeting vector.

In another aspect of the present invention, ungulate cells lacking at least one allele of a functional region of an ungulate heavy chain, kappa light chain and/or lambda light chain locus produced according to the process, sequences and/or constructs described herein are provided. These cells can be obtained as a result of homologous recombination. Particularly, by inactivating at least one allele of an ungulate heavy chain, kappa light chain or lambda light chain gene, cells can be produced which have reduced capability for expression of ungulate antibodies. In other embodiments, mammalian cells lacking both alleles of an ungulate heavy chain, kappa light chain and/or lambda light chain gene can be produced according to the process, sequences and/or constructs described herein. In a further embodiment, porcine animals are provided in which at least one allele of an ungulate heavy chain, kappa light chain and/or lambda light chain gene is inactivated via a genetic targeting event produced according to the process, sequences and/or constructs described herein. In another aspect of the present invention, porcine animals are provided in which both alleles of an ungulate heavy chain, kappa light chain and/or lambda light chain gene are inactivated via a genetic targeting event. The gene can be targeted via homologous recombination.

In other embodiments, the gene can be disrupted, i.e. a portion of the genetic code can be altered, thereby affecting transcription and/or translation of that segment of the gene. For example, disruption of a gene can occur through substitution, deletion (“knock-out”) or insertion (“knock-in”) techniques. Additional genes for a desired protein or regulatory sequence that modulate transcription of an existing sequence can be inserted. To achieve multiple genetic modifications of ungulate immunoglobulin genes, in one embodiment, cells can be modified sequentially to contain multiple genentic modifications. In other embodiments, animals can be bred together to produce animals that contain multiple genetic modifications of immunoglobulin genes. As an illustrative example, animals that lack expression of at least one allele of an ungulate heavy chain gene can be further genetically modified or bred with animals lacking at least one allele of a kappa light chain gene.

In embodiments of the present invention, alleles of ungulate heavy chain, kappa light chain or lambda light chain gene are rendered inactive according to the process, sequences and/or constructs described herein, such that functional ungulate immunoglobulins can no longer be produced. In one embodiment, the targeted immunoglobulin gene can be transcribed into RNA, but not translated into protein. In another embodiment, the targeted immunoglobulin gene can be transcribed in an inactive truncated form. Such a truncated RNA may either not be translated or can be translated into a nonfunctional protein. In an alternative embodiment, the targeted immunoglobulin gene can be inactivated in such a way that no transcription of the gene occurs. In a further embodiment, the targeted immunoglobulin gene can be transcribed and then translated into a nonfunctional protein.

In a further aspect of the present invention, ungulate, such as porcine or bovine, cells lacking one allele, optionally both alleles of an ungulate heavy chain, kappa light chain and/or lambda light chain gene can be used as donor cells for nuclear transfer into recipient cells to produce cloned, transgenic animals. Alternatively, ungulate heavy chain, kappa light chain and/or lambda light chain gene knockouts can be created in embryonic stem cells, which are then used to produce offspring. Offspring lacking a single allele of a functional ungulate heavy chain, kappa light chain and/or lambda light chain gene produced according to the process, sequences and/or constructs described herein can be breed to further produce offspring lacking functionality in both alleles through mendelian type inheritance.

In one aspect of the present invention, a method is provided to disrupt the expression of an ungulate immunoglobulin gene by (i) analyzing the germline configuration of the ungulate heavy chain, kappa light chain or lambda light chain genomic locus; (ii) determining the location of nucleotide sequences that flank the 5′ end and the 3′ end of at least one functional region of the locus; and (iii) transfecting a targeting construct containing the flanking sequence into a cell wherein, upon successful homologous recombination, at least one functional region of the immunoglobulin locus is disrupted thereby reducing or preventing the expression of the immunoglobulin gene. In one embodiment, the germline configuration of the porcine heavy chain locus is provided. The porcine heavy chain locus contains at least four variable regions, two diversity regions, six joining regions and five constant regions, for example, as illustrated in FIG. 1. In a specific embodiment, only one of the six joining regions, J6, is functional. In another embodiment, the germline configuration of the porcine kappa light chain locus is provided. The porcine kappa light chain locus contains at least six variable regions, six joining regions, one constant region and one enhancer region, for example, as illustrated in FIG. 2. In a further embodiment, the germline configuration of the porcine lambda light chain locus is provided.

In further aspects of the present invention provides ungulates and ungulate cells that lack at least one allele of a functional region of an ungulate heavy chain, kappa light chain and/or lambda light chain locus produced according to the processes, sequences and/or constructs described herein, which are further modified to express at least part of a human antibody (i.e. immunoglobulin (Ig)) locus. In additional embodiments, porcine animals are provided that express xenogenous immunoglobulin. This human locus can undergoe rearrangement and express a diverse population of human antibody molecules in the ungulate. These cloned, transgenic ungulates provide a replenishable, theoretically infinite supply of human antibodies (such as polyclonal antibodies), which can be used for therapeutic, diagnostic, purification, and other clinically relevant purposes. In one particular embodiment, artificial chromosomes (ACs), such as yeast or mammalian artificial chromosomes (YACS or MACS) can be used to allow expression of human immunoglobulin genes into ungulate cells and animals. All or part of human immunoglobulin genes, such as the Ig heavy chain gene (human chromosome 414), Ig kappa chain gene (human chromosome #2) and/or the Ig lambda chain gene (chromosome #22) can be inserted into the artificial chromosomes, which can then be inserted into ungulate cells. In further embodiments, ungulates and ungulate cells are provided that contain either part or all of at least one human antibody gene locus, which undergoes rearrangement and expresses a diverse population of human antibody molecules.

In additional embodiments, methods of producing xenogenous antibodies are provided, wherein the method can include: (a) administering one or more antigens of interest to an ungulate whose cells comprise one or more artificial chromosomes and lack any expression of functional endogenous immunoglobulin, each artificial chromosome comprising one or more xenogenous immunoglobulin loci that undergo rearrangement, resulting in production of xenogenous antibodies against the one or more antigens; and/or (b) recovering the xenogenous antibodies from the ungulate. In one embodiment, the immunoglobulin loci can undergo rearrangement in a B cell.

In one aspect of the present invention, an ungulate, such as a pig or a cow, can be prepared by a method in accordance with any aspect of the present invention. These cloned, transgenic ungulates (e.g., porcine and bovine animals) provide a replenishable, theoretically infinite supply of human polyclonal antibodies, which can be used as therapeutics, diagnostics and for purification purposes. For example, transgenic animals produced according to the process, sequences and/or constructs described herein that produce polyclonal human antibodies in the bloodstream can be used to produce an array of different antibodies which are specific to a desired antigen. The availability of large quantities of polyclonal antibodies can also be used for treatment and prophylaxis of infectious disease, vaccination against biological warfare agents, modulation of the immune system, removal of undesired human cells such as cancer cells, and modulation of specific human molecules.

In other embodiments, animals or cells lacking expression of functional immunoglobulin, produced according to the process, sequences and/or constructs described herein, can contain additional genetic modifications to eliminate the expression of xenoantigens. Such animals can be modified to elimate the expression of at least one allele of the alpha-1,3-galactosyltransferase gene, the CMP-Neu5Ac hydroxylase gene (see, for example, U.S. Ser. No. 10/863,116), the iGb3 synthase gene (see, for example, U.S. Patent Application 60/517,524), and/or the Forssman synthase gene (see, for example, U.S. Patent Application 60/568,922). In additional embodiments, the animals discloses herein can also contain genetic modifications to expresss fucosyltransferase and/or sialyltransferase. To achieve these additional genetic modifications, in one embodiment, cells can be modified to contain multiple genentic modifications. In other embodiments, animals can be bred together to achieve multiple genetic modifications. In one specific embodiment, animals, such as pigs, lacking expression of functional immunoglobulin, produced according to the process, sequences and/or constructs described herein, can be bred with animals, such as pigs, lacking expression of alpha-1,3-galactosyl transferase (for example, as described in WO 04/028243).

### BRIEF DESCRIPTION OF THE DRAWINGS

FIG. 1 illustrates the design of a targeting vector that disrupts the expression of the joining region of the porcine heavy chain immunoglobulin gene.

FIG. 2 illustrates the design of a targeting vector that disrupts the expression of the constant region of the porcine kappa light chain immunoglobulin gene.

FIG. 3 illustrates the genomic organization of the porcine lambda immunoglobulin locus, including a concatamer of J-C sequences as well as flanking regions that include the variable region 5′ to the JC region. Bacterial artificial chromosomes (BAC1 and BAC2) represent fragments of the porcine immunoglobulin genome that can be obtained from BAC libraries.

FIG. 4 represents the design of a targeting vector that disrupts the expression of the JC clusterregion of the porcine lambda light chain immunoglobulin gene. “SM” stands for a selectable marker gene, which can be used in the targeting vector.

FIG. 5 illustrates a targeting strategy to insert a site specific recombinase target or recognition site into the region 5′ of the JC cluster region of the porcine lambda immunoglobulin locus. “SM” stands for a selectable marker gene, which can be used in the targeting vector. “SSRRS” stands for a specific recombinase target or recognition site.

FIG. 6 illustrates a targeting strategy to insert a site specific recombinase target or recognition site into the region 3′ of the JC cluster region of the porcine lambda immunoglobulin locus. “SM” stands for a selectable marker gene, which can be used in the targeting vector. “SSRRS” stands for a specific recombinase target or recognition site.

FIG. 7 illustrates the site specific recombinase mediated transfer of a YAC into a host genome. “SSRRS” stands for a specific recombinase target or recognition site.

### DETAILED DESCRIPTION

The present invention provides for the first time ungulate immunoglobin germline gene sequence arrangement as well as novel genomic sequences thereof. In addition, novel ungulate cells, tissues and animals that lack at least one allele of a heavy or light chain immunoglobulin gene are provided. Based on this discovery, ungulates can be produced that completely lack at least one allele of a heavy and/or light chain immunoglobulin gene. In addition, these ungulates can be further modified to express xenoogenous, such as human, immunoglobulin loci or fragments thereof.

In one aspect of the present invention, a transgenic ungulate that lacks any expression of functional endogenous immunoglobulins is provided. In one embodiment, the ungulate can lack any expression of endogenous heavy and/or light chain immunoglobulins. The light chain immunoglobulin can be a kappa and/or lambda immunoglobulin. In additional embodiments, transgenic ungulates are provided that lack expression of at least one allele of an endogenous immunoglobulin wherein the immunoglobulin is selected from the group consisting of heavy chain, kappa light chain and lambda light chain or any combination thereof. In one embodiment, the expression of functional endogenous immunoglobulins can be accomplished by genetic targeting of the endogenous immunoglobulin loci to prevent expression of the endogenous immunoglobulin. In one embodiment, the genetic targeting can be accomplished via homologous recombination. In another embodiment, the transgenic ungulate can be produced via nuclear transfer.

In other embodiments, the transgenic ungulate that lacks any expression of functional endogenous immunoglobulins can be further genetically modified to express an xenogenous immunoglobulin loci. In an alternative embodiment, porcine animals are provided that contain an xenogeous immunoglobulin locus. In one embodiment, the xenogeous immunoglobulin loci can be a heavy and/or light chain immunoglobulin or fragment thereof. In another embodiment, the xenogenous immunoglobulin loci can be a kappa chain locus or fragment thereof and/or a lambda chain locus or fragment thereof. In still further embodiments, an artificial chromosome (AC) can contain the xenogenous immunoglobulin. In one embodiment, the AC can be a yeast AC or a mammalian AC. In a further embodiment, the xenogenous locus can be a human immunoglobulin locus or fragment thereof. In one embodiment, the human immunoglobulin locus can be human chromosome 14, human chromosome 2, and human chromosome 22 or fragments thereof. In another embodiment, the human immunoglobulin locus can include any fragment of a human immunoglobulin that can undergo rearrangement. In a further embodiment, the human immunoglobulin loci can include any fragment of a human immunoglobulin heavy chain and a human immunoglobulin light chain that can undergo rearrangement. In still further embodiment, the human immunoglobulin loci can include any human immunoglobulin locus or fragment thereof that can produce an antibody upon exposure to an antigen. In a particular embodiment, the exogenous human immunoglobulin can be expressed in B cells to produce xenogenous immunoglobulin in response to exposure to one or more antigens.

In another aspect of the present invention, transgenic ungulates are provided that expresses a xenogenous immunoglobulin loci or fragment thereof, wherein the immunoglobulin can be expressed from an immunoglobulin locus that is integrated within an endogenous ungulate chromosome. In one embodiment, ungulate cells derived from the transgenic animals are provided. In one embodiment, the xenogenous immunoglobulin locus can be inherited by offspring. In another embodiment, the xenogenous immunoglobulin locus can be inherited through the male germ line by offspring. In still further embodiments, an artificial chromosome (AC) can contain the xenogenous immunoglobulin. In one embodiment, the AC can be a yeast AC or a mammalian AC. In a further embodiment, the xenogenous locus can be a human immunoglobulin locus or fragment thereof. In one embodiment, the human immunoglobulin locus can be human chromosome 14, human chromosome 2, and human chromosome 22 or fragments thereof. In another embodiment, the human immunoglobulin locus can include any fragment of a human immunoglobulin that can undergo rearrangement. In a further embodiment, the human immunoglobulin loci can include any fragment of a human immunoglobulin heavy chain and a human immunoglobulin light chain that can undergo rearrangement. In still further embodiment, the human immunoglobulin loci can include any human immunoglobulin locus or fragment thereof that can produce an antibody upon exposure to an antigen. In a particular embodiment, the exogenous human immunoglobulin can be expressed in B cells to produce xenogenous immunoglobulin in response to exposure to one or more antigens.

Definitions

The terms “recombinant DNA technology,” “DNA cloning,” “molecular cloning,” or “gene cloning” refer to the process of transferring a DNA sequence into a cell or orgaism. The transfer of a DNA fragment can be from one organism to a self-replicating genetic element (e.g., bacterial plasmid) that permits a copy of any specific part of a DNA (or RNA) sequence to be selected among many others and produced in an unlimited amount. Plasmids and other types of cloning vectors such as artificial chromosomes can be used to copy genes and other pieces of chromosomes to generate enough identical material for further study. In addition to bacterial plasmids, which can carry up to 20 kb of foreign DNA, other cloning vectors include viruses, cosmids, and artificial chromosomes (e.g., bacteria artificial chromosomes (BACs) or yeast artificial chromosomes (YACs)). When the fragment of chromosomal DNA is ultimately joined with its cloning vector in the lab, it is called a “recombinant DNA molecule.” Shortly after the recombinant plasmid is introduced into suitable host cells, the newly inserted segment will be reproduced along with the host cell DNA.

“Cosmids” are artificially constructed cloning vectors that carry up to 45 kb of foreign DNA. They can be packaged in lambda phage particles for infection into E. coli cells.

As used herein, the term “mammal” (as in “genetically modified (or altered) mammal”) is meant to include any non-human mammal, including but not limited to pigs, sheep, goats, cattle (bovine), deer, mules, horses, monkeys, dogs, cats, rats, mice, birds, chickens, reptiles, fish, and insects. In one embodiment of the invention, genetically altered pigs and methods of production thereof are provided.

The term “ungulate” refers to hoofed mammals. Artiodactyls are even-toed (cloven-hooved) ungulates, including antelopes, camels, cows, deer, goats, pigs, and sheep. Perissodactyls are odd toes ungulates, which include horses, zebras, rhinoceroses, and tapirs. The term ungulate as used herein refers to an adult, embryonic or fetal ungulate animal.

As used herein, the terms “porcine”, “porcine animal”, “pig” and “swine” are generic terms referring to the same type of animal without regard to gender, size, or breed.

A “homologous DNA sequence or homologous DNA” is a DNA sequence that is at least about 80%, 85%, 90%, 95%, 98% or 99% identical with a reference DNA sequence. A homologous sequence hybridizes under stringent conditions to the target sequence, stringent hybridization conditions include those that will allow hybridization occur if there is at least 85, at least 95% or 98% identity between the sequences.

An “isogenic or substantially isogenic DNA sequence” is a DNA sequence that is identical to or nearly identical to a reference DNA sequence. The term “substantially isogenic” refers to DNA that is at least about 97-99% identical with the reference DNA sequence, or at least about 99.5-99.9% identical with the reference DNA sequence, and in certain uses 100% identical with the reference DNA sequence.

“Homologous recombination” refers to the process of DNA recombination based on sequence homology.

“Gene targeting” refers to homologous recombination between two DNA sequences, one of which is located on a chromosome and the other of which is not.

“Non-homologous or random integration” refers to any process by which DNA is integrated into the genome that does not involve homologous recombination.

A “selectable marker gene” is a gene, the expression of which allows cells containing the gene to be identified. A selectable marker can be one that allows a cell to proliferate on a medium that prevents or slows the growth of cells without the gene. Examples include antibiotic resistance genes and genes which allow an organism to grow on a selected metabolite. Alternatively, the gene can facilitate visual screening of transformants by conferring on cells a phenotype that is easily identified. Such an identifiable phenotype can be, for example, the production of luminescence or the production of a colored compound, or the production of a detectable change in the medium surrounding the cell.

The term “contiguous” is used herein in its standard meaning, i.e., without interruption, or uninterrupted.

“Stringent conditions” refers to conditions that (1) employ low ionic strength and high temperature for washing, for example, 0.015 M NaCl/0.0015 M sodium citrate/0.1% SDS at 50° C., or (2) employ during hybridization a denaturing agent such as, for example, formamide. One skilled in the art can determine and vary the stringency conditions appropriately to obtain a clear and detectable hybridization signal. For example, stringency can generally be reduced by increasing the salt content present during hybridization and washing, reducing the temperature, or a combination thereof. See, for example, Sambrook et al., Molecular Cloning: A Laboratory Manual, Cold Spring Harbour Laboratory Press, Cold Spring Harbour, New York, (1989).

I. Immunoglobulin Genes

In one aspect of the present invention, a transgenic ungulate that lacks any expression of functional endogenous immunoglobulins is provided. In one embodiment, the ungulate can lack any expression of endogenous heavy and/or light chain immunoglobulins. The light chain immunoglobulin can be a kappa and/or lambda immunoglobulin. In additional embodiments, transgenic ungulates are provided that lack expression of at least one allele of an endogenous immunoglobulin wherein the immunoglobulin is selected from the group consisting of heavy chain, kappa light chain and lambda light chain or any combination thereof. In one embodiment, the expression of functional endogenous immunoglobulins can be accomplished by genetic targeting of the endogenous immunoglobulin loci to prevent expression of the endogenous immunoglobulin. In one embodiment, the genetic targeting can be accomplished via homologous recombination. In another embodiment, the transgenic ungulate can be produced via nuclear transfer.

In another aspect of the present invention, a method is provided to disrupt the expression of an ungulate immunoglobulin gene by (i) analyzing the germline configuration of the ungulate heavy chain, kappa light chain or lambda light chain genomic locus; (ii) determining the location of nucleotide sequences that flank the 5′ end and the 3′ end of at least one functional region of the locus; and (iii) transfecting a targeting construct containing the flanking sequence into a cell wherein, upon successful homologous recombination, at least one functional region of the immunoglobulin locus is disrupted thereby reducing or preventing the expression of the immunoglobulin gene.

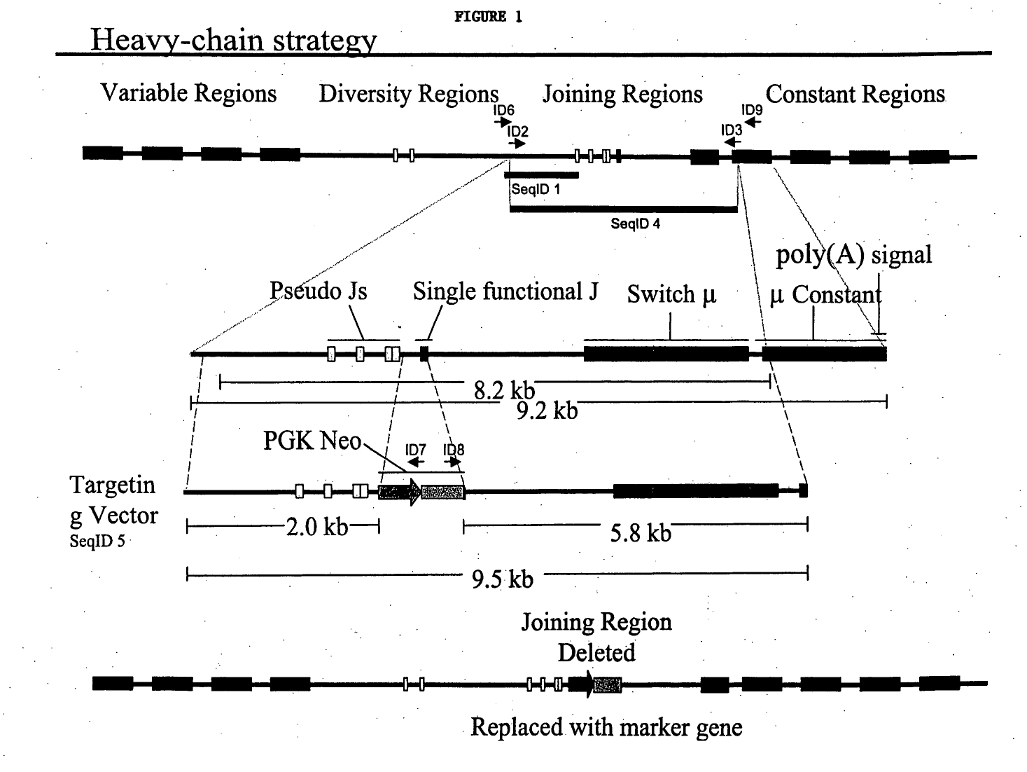

In one embodiment, the germline configuration of the porcine heavy chain locus is provided. The porcine heavy chain locus contains at least four variable regions, two diversity regions, six joining regions and five constant regions, for example, as illustrated in FIG. 1. In a specific embodiment, only one of the six joining regions, J6, is functional.

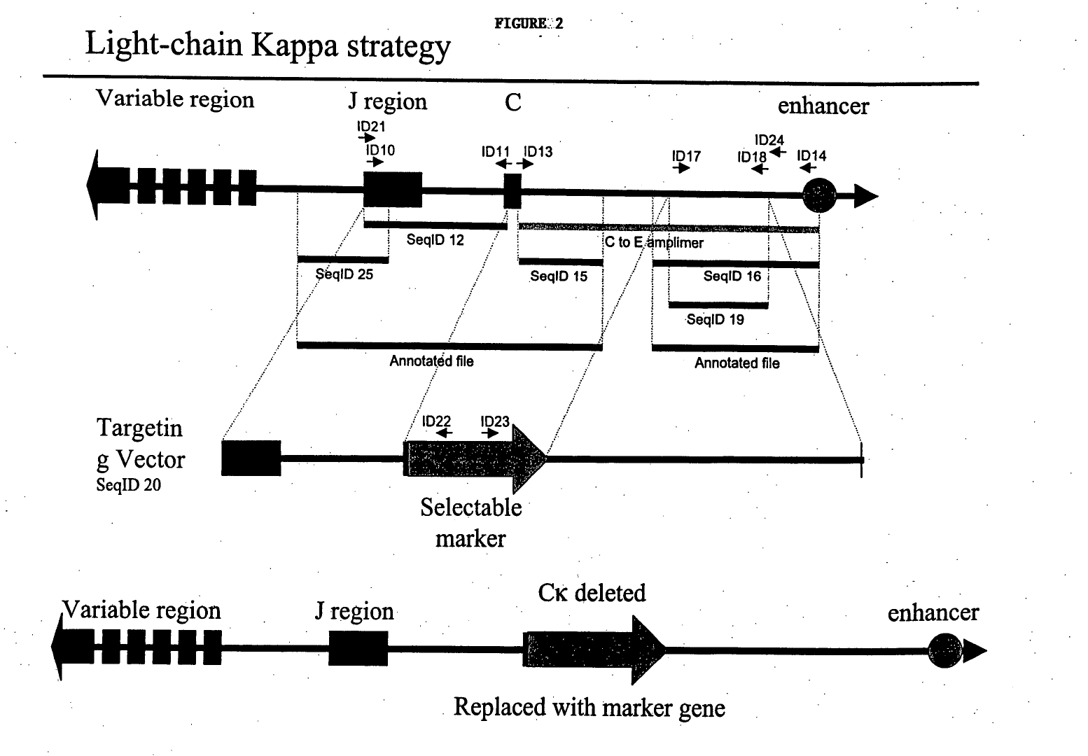

In another embodiment, the germline configuration of the porcine kappa light chain locus is provided. The porcine kappa light chain locus contains at least six variable regions, six joining regions, one constant region and one enhancer region, for example, as illustrated in FIG. 2.

In a further embodiment, the germline configuration of the porcine lambda light chain locus is provided.

Isolated nucleotide sequences as depicted in Seq ID Nos 1-39 are provided. Nucleic acid sequences at least 80, 85, 90, 95, 98 or 99% homologous to any one of Seq ID Nos 1-39 are also provided. In addition, nucleotide sequences that contain at least 10, 15, 17, 20, 25 or 30 contiguous nucleotides of any one of Seq ID Nos 1-39 are provided. Further provided are nucleotide sequences that hybridizes, optionally under stringent conditions, to Seq ID Nos 1-39, as well as, nucleotides homologous thereto.

Homology or identity at the nucleotide or amino acid sequence level can be determined by BLAST (Basic Local Alignment Search Tool) analysis using the algorithm employed by the programs blastp, blastn, blastx, tblastn and tblastx (see, for example, Altschul, S. F. et al (1 997) Nucleic Acids Res 25:3389-3402 and Karlin et al, (1 900) Proc. Natl. Acad. Sci. USA 87, 2264-2268) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a preselected threshold of significance. See, for example, Altschul et al., (1994) (Nature Genetics 6, 119-129). The search parameters for histogram, descriptions, alignments, expect (ie., the statistical significance threshold for reporting matches against database sequences), cutoff, matrix and filter (low co M'plexity) are at the default settings. The default scoring matrix used by blastp, blastx, tblastn, and tblastx is the BLOSUM62 matrix (Henikoff et al., (1 992) Proc. Natl. Acad. Sci. USA 89, 10915-10919), which is recommended for query sequences over 85 in length (nucleotide bases or amino acids).

Porcine Heavy Chain

In another aspect of the present invention, novel genomic sequences encoding the heavy chain locus of ungulate immunoglobulin are provided. In one embodiment, an isolated nucleotide sequence encoding porcine heavy chain is provided that includes at least one variable region, two diversity regions, at least four joining regions and at least one constant region, such as the mu constant region, for example, as represented in Seq ID No. 29. In another embodiment, an isolated nucleotide sequence is provided that includes at least four joining regions and at least one constant region, such as as the mu constant region, of the porcine heavy chain genomic sequence, for example, as represented in Seq ID No. 4. In a further embodiment, nucleotide sequence is provided that includes 5′ flanking sequence to the first joining region of the porcine heavy chain genomic sequence, for example, as represented in Seq ID No 1. Still further, nucleotide sequence is provided that includes 3′ flanking sequence to the first joining region of the porcine heavy chain genomic sequence, for example, as represented in the 3′ region of Seq ID No 4. In further embodiments, isolated nucleotide sequences as depicted in Seq ID Nos 1, 4 or 29 are provided. Nucleic acid sequences at least 80, 85, 90, 95, 98 or 99% homologous to Seq ID Nos 1, 4 or 29 are also provided. Further provided are nucleotide sequences that hybridizes, optionally under stringent conditions, to Seq ID Nos 1, 4 or 29, as well as, nucleotides homologous thereto.

In addition, nucleotide sequences that contain at least 10, 15, 17, 20, 25 or 30 contiguous nucleotides of Seq ID Nos 1, 4 or 29 are provided. In one embodiment, the nucleotide sequence contains at least 17, 20, 25 or 30 contiguous nucleotides of Seq ID No 4 or residues 1-9,070 of Seq ID No 29. In other embodiments, nucleotide sequences that contain at least 50, 100, 1,000, 2,500, 4,000, 4,500, 5,000, 7,000, 8,000, 8,500, 9,000, 10,000 or 15,000 contiguous nucleotides of Seq ID No. 29 are provided. In another embodiment, the nucleotide sequence contains residues 9,070-11039 of Seq ID No 29.

In further embodiments, isolated nucleotide sequences as depicted in Seq ID Nos 1, 4 or 29 are provided. Nucleic acid sequences at least 80, 85, 90, 95, 98 or 99% homologous to Seq ID Nos 1, 4 or 29 are also provided. In addition, nucleotide sequences that contain at least 10, 15, 17, 20, 25 or 30 contiguous nucleotides of Seq ID Nos 1, 4 or 29 are provided. Further provided are nucleotide sequences that hybridizes, optionally under stringent conditions, to Seq ID Nos 1, 4 or 29, as well as, nucleotides homologous thereto.

In one embodiment, an isolated nucleotide sequence encoding porcine heavy chain is provided that includes at least one variable region, two diversity regions, at least four joining regions and at least one constant region, such as the mu constant region, for example, as represented in Seq ID No. 29. In Seq ID No. 29, the Diversity region of heavy chain is represented, for example, by residues 1089-1099 (D(pseudo)), the Joining region of heavy chain is represented, for example, by residues 1887-3352 (for example: J(psuedo): 1887-1931, J(psuedo): 2364-2411, J(psuedo): 2756-2804, J (functional J): 3296-3352), the recombination signals are represented, for example, by residues 3001-3261 (Nonamer), 3292-3298 (Heptamer), the Constant Region is represented by the following residues: 3353-9070 (J to C mu intron), 5522-8700 (Switch region), 9071-9388 (Mu Exon 1), 9389-9469 (Mu Intron A), 9470-9802 (Mu Exon 2), 9830-10069 (Mu Intron B), 10070-10387 (Mu Exon 3), 10388-10517 (Mu Intron C), 10815-11052 (Mu Exon 4), 11034-11039 (Poly(A) signal).

| | |
|---|---|
| Seq ID No. 29 | tctagaagacgctggagagaggccagacttcctcgga |
| | acagctcaaagagctctgtcaaagccagatcccatca |
| | cacgtgggcaccaataggccatgccagcctccaaggg |
| | ccgaactgggttctccacggcgcacatgaagcctgca |
| | gcctggcttatcctcttccgtggtgaagaggcaggcc |
| | cgggactggacgaggggctagcagggtgtggtaggca |
| | ccttgcgccccccaccccggcaggaaccagagaccct |
| | ggggctgagagtgagcctccaaacaggatgccccacc |
| | cttcaggccacctttcaatccagctacactccacctg |
| | ccattctcctctgggcacagggcccagcccctggatc |
| | ttggccttggctcgacttgcacccacgcgcacacaca |
| | cacttcctaacgtgctgtccgctcacccctccccagc |
| | gtggtccatgggcagcacggcagtgcgcgtccggcgg |
| | tagtgagtgcagaggtcccttcccctcccccaggagc |
| | cccaggggtgtgtgcagatctgggggctcctgtccct |
| | tacaccttcatgcccctcccctcatacccaccctcca |
| | ggcgggaggcagcgagacctttgcccagggactcagc |
| | caacgggcacacgggaggccagccctcagcagctggc |
| | tcccaaagaggaggtgggaggtaggtccacagctgcc |
| | acagagagaaaccctgacggaccccacaggggccacg |
| | ccagccggaaccagctccctcgtgggtgagcaatggc |
| | cagggccccgccggccaccacggctggccttgcgcca |
| | gctgagaactcacgtccagtgcagggagactcaagac |
| | agcctgtgcacacagcctcggatctgctcccatttca |
| | agcagaaaaaggaaaccgtgcaggcagccctcagcat |
| | ttcaaggattgtagcagcggccaactattcgtcggca |
| | gtggccgattagaatgaccgtggagaagggcggaagg |
| | gtctctcgtgggctctgcggccaacaggccctggctc |
| | cacctgcccgctgccagcccgaggggcttgggccgag |
| | ccaggaaccacagtgctcaccgggaccacagtgactg |
| | accaaactcccggccagagcagccccaggccagccgg |
| | gctctcgccctggaggactcaccatcagatgcacaag |
| | ggggcgagtgtggaagagacgtgtcgcccgggccatt |
| | tgggaaggcgaagggaccttccaggtggacaggaggt |
| | gggacgcactccaggcaagggactgggtccccaaggc |
| | ctggggaaggggtactggcttgggggttagcctggcc |
| | agggaacggggagcggggcggggggctgagcagggag |
| | gacctgacctcgtgggagcgaggcaagtcaggcttca |
| | ggcagcagccgcacatcccagaccaggaggctgaggc |
| | aggaggggcttgcagcggggcgggggcctgcctggct |
| | ccgggggctcctgggggacgctggctcttgtttccgt |
| | gtcccgcagcacagggccagctcgctgggcctatgct |
| | taccttgatgtctggggccggggcgtcagggtcgtcg |
| | tctcctcaggggagagtcccctgaggctacgctgggg |
| | *ggggactatggcagctccaccaggggcctggggacc |
| | aggggcctggaccaggctgcagcccggaggacgggca |
| | gggctctggctctccagcatctggccctcggaaatgg |
| | cagaacccctggcgggtgagcgagctgagagcgggtc |
| | agacagacaggggccggccggaaaggagaagttgggg |
| | gcagagcccgccaggggccaggcccaaggttctgtgt |
| | gccagggcctgggtgggcacattggtgtggccatggc |
| | tacttagattcgtggggccagggcatcctggtcaccg |
| | tctcctcaggtgagcctggtgtctgatgtccagctag |
| | gcgctggtgggccgcgggtgggcctgtctcaggctag |
| | ggcaggggctgggatgtgtatttgtcaaggaggggca |
| | acagggtgcagactgtgcccctggaaacttgaccact |
| | ggggcaggggcgtcctggtcacgtctcctcaggtaag |
| | acggccctgtgcccctctctcgcgggactggaaaagg |
| | aattttccaagattccttggtctgtgtggggccctct |
| | ggggcccccgggggtggctcccctcctgcccagatgg |
| | ggcctcggcctgtggagcacgggctgggcacacagct |
| | cgagtctagggccacagaggcccgggctcagggctct |
| | gtgtggcccggcgactggcagggggctcgggtttttg |
| | gacaccccctaatgggggccacagcactgtgaccatc |
| | ttcacagctggggccgaggagtcgaggtcaccgtctc |
| | ctcaggtgagtcctcgtcagccctctctcactctctg |
| | gggggttttgctgcattttgtgggggaaagaggatgc |
| | ctgggtctcaggtctaaaggtctagggccagcgccgg |
| | ggcccaggaaggggccgaggggccaggctcggctcgg |
| | ccaggagcagagcttccagacatctcgcctcctggcg |
| | gctgcagtcaggcctttggccgggggggtctcagcac |
| | caccaggcctcttggctcccgaggtccccggccccgg |
| | ctgcctcaccaggcaccgtgcgcggtgggcccgggct |
| | cttggtcggccaccctttcttaactgggatccgggct |
| | tagttgtcgcaatgtgacaacgggctcgaaagctggg |
| | gccaggggaccctagtctacgacgcctcgggtgggtg |
| | tcccgcacccctccccactttcacggcactcggcgag |
| | acctggggagtcaggtgttggggacactttggaggtc |
| | aggaacgggagctggggagagggctctgtcagcgggg |
| | tccagagatgggccgccctccaaggacgccctgcgcg |
| | gggacaagggcttcttggcctggcctggccgcttcac |
| | ttgggcgtcagggggggcttcccggggcaggcggtca |
| | gtcgaggcgggttggaattctgagtctgggttcgggg |
| | ttcggggttcggccttcatgaacagacagcccaggcg |
| | ggccgttgtttggcccctgggggcctggttggaatgc |
| | gaggtctcgggaagtcaggagggagcctggccagcag |
| | agggttcccagccctgcggccgagggacctggagacg |
| | ggcagggcattggccgtcgcagggccaggccacaccc |
| | cccaGGTTTTTGTggggcgagcctggagattgcacCA |
| | CTGTGATTACTATGCTATGGATCTCTGGGGCCGAGGC |
| | GTTGAAGTCGTCGTGTGCTCAGgtaagaacggccctc |
| | cagggcctttaatttctgctctcgtctgtgggctttt |
| | ctgactctgatcctcgggaggcgtctgtgcccccccc |
| | ggggatgaggccggcttgccaggaggggtcagggacc |
| | aggagcctgtgggaagttctgacgggggctgcaggcg |
| | ggaagggccccaccggggggcgagccccaggccgctg |
| | ggcggcaggagacccgtgagagtgcgccttgaggagg |
| | gtgtctgcggaaccacgaacgcccgccgggaagggct |
| | tgctgcaatgcggtcttcagacgggaggcgtcttctg |
| | ccctcaccgtctttcaagcccttgtgggtctgaaaga |
| | gccatgtcggagagagaagggacaggcctgtcccgac |
| | ctggccgagagcgggcagccccgggggagagcggggc |
| | gatcggcctgggctctgtgaggccaggtccaagggag |
| | gacgtgtggtcctcgtgacaggtgcacttgcgaaacc |
| | ttagaagacggggtatgttggaagcggctcctgatgt |
| | ttaagaaaagggagactgtaaagtgagcagagtcctc |
| | aagtgtgttaaggttttaaaggtcaaagtgttttaaa |
| | cctttgtgactgcagttagcaagcgtgcggggagtga |
| | atggggtgccagggtggccgagaggcagtacgagggc |
| | cgtgccgtcctctaattcagggcttagttttgcagaa |
| | taaagtcggcctgttttctaaaagcattggtggtgct |
| | gagctggtggaggaggccgcgggcagccctggccacc |
| | tgcagcagggtggcaggaagcaggtcggccaagaggc |
| | tatttaggaagccagaaaacacggtcgatgaatttat |
| | agcttctggtttccaggaggtggttgggcatggcttt |
| | gcgcagcgccacagaaccgaaagtgcccactgagaaa |
| | aaacaactcctgcttaatttgcatttttctaaaagaa |
| | gaaacagaggctgacggaaactggaaagttcctgttt |
| | taactactcgaattgagttttcggtcttagcttatca |
| | actgctcacttagattcattttcaaagtaaacgttta |
| | agagccgaggcattcctatcctcttctaaggcgttat |
| | tcctggaggctcattcaccgccagcacctccgctgcc |
| | tgcaggcattgctgtcaccgtcaccgtgacggcgcgc |
| | acgattttcagttggcccgcttcccctcgtgattagg |
| | acagacgcgggcactctggcccagccgtcttggctca |
| | gtatctgcaggcgtccgtctcgggacggagctcaggg |
| | gaagagcgtgactccagttgaacgtgatagtcggtgc |
| | gttgagaggagacccagtcgggtgtcgagtcagaagg |
| | ggcccggggcccgaggccctgggcaggacggcccgtg |
| | ccctgcatcacgggcccagcgtcctagaggcaggact |
| | ctggtggagagtgtgagggtgcctggggcccctccgg |
| | agctggggccgtgcggtgcaggttgggctctcggcgc |
| | ggtgttggctgtttctgcgggatttggaggaattctt |
| | ccagtgatgggagtcgccagtgaccgggcaccaggct |
| | ggtaagagggaggccgccgtcgtggccagagcagctg |
| | ggagggttcggtaaaaggctcgcccgtttcctttaat |
| | gaggacttttcctggagggcatttagtctagtcggga |
| | ccgttttcgactcgggaagagggatgcggaggagggc |
| | atgtgcccaggagccgaaggcgccgcggggagaagcc |
| | cagggctctcctgtccccacagaggcgacgccactgc |
| | cgcagacagacagggcctttccctctgatgacggcaa |
| | aggcgcctcggctcttgcggggtgctgggggggagtc |
| | gccccgaagccgctcacccagaggcctgaggggtgag |
| | actgaccgatgcctcttggccgggcctggggccggac |
| | cgagggggactccgtggaggcagggcgatggtggctg |
| | cgggagggaaccgaccctgggccgagcccggcttggc |
| | gattcccgggcgagggccctcagccgaggcgagtggg |
| | tccggcggaaccaccctttctggccagcgccacaggg |
| | ctctcgggactgtccggggcgacgctgggctgcccgt |
| | ggcaggccTGGGCTGACGTGGACTTCACCAGACAGAA |
| | CAGGGCTTTCAGGGCTGAGCTGAGCCAGGTTTAGCGA |
| | GGCCAAGTGGGGCTGAACGAGGCTGAACTGGGCTGAG |
| | CTGGGTTGAGCTGGGCTGACCTGGGCTGAGGTGAGCT |
| | GGGCTGGGCTGGGCTGGGCTGGGCTGGGCTGGGCTGG |
| | ACTGGCTGAGCTGAGCTGGGTTGAGCTGAGCTGAGCT |
| | GGCCTGGGTTGAGCTGGGCTGGGTTGAGCTGAGCTGG |
| | GTTGAGCTGGGTTGAGCTGGGTTGATCTGAGCTGAGG |
| | TGGGCTGAGCTGAGCTAGGCTGGGGTGAGCTGGGCTG |
| | AGCTGGTTTGAGTTGGGTTGAGCTGAGGTGAGGTGGG |
| | CTGTGGTGGCTGAGGTAGGCTGAGGTAGGCTAGGTTG |
| | AGGTGGGGTGGGCTGAGCTGAGCTAGGCTGGGCTGAT |
| | TTGGGCTGAGCTGAGCTGAGCTAGGCTGCGTTGAGCT |
| | GGCTGGGCTGGATTGAGCTGGCTGAGCTGGCTGAGCT |
| | GGGCTGAGGTGGCGTGGGTTGAGCTGAGCTGGACTGG |
| | TTTGAGCTGGGTCGATCTGGGTTGAGCTGTCCTGGGT |
| | TGAGCTGGGCTGGGTTGAGCTGAGCTGGGTTGAGCTG |
| | GGCTCAGCAGAGCTGGGTTGGGCTGAGCTGGGTTGAG |
| | CTGAGCTGGGGTGAGCTGGCCTGGGTTGAGCTGGGCT |
| | GAGCTGAGCTGGGCTGAGCTGGCGTGTGCTGAGCTGG |
| | GCTGGGTTGAGCTGGGCTGAGGTGGATTGAGCTGGGT |
| | TGAGCTGAGCTGGGGTGGGCTGTGCTGACTGAGCTGG |
| | GGTGAGCTAGGCTGGGGTGAGCTGGGCTGAGCTGATC |
| | CGAGGTAGGCTGGGGTGGTATGGGCTGAGCTGAGCTG |
| | AGCTAGGCTGGATTGATCTGGCTGAGCTGGGTTGAGC |
| | TGAGCTGGGCTGAGCTGGTCTGAGCTGGGCTGGGTCG |
| | AGCTGAGCTGGACTGGTTTGAGCTGGGTCGATCTGGG |
| | CTGAGCTGGCCTGGGTTGAGCTGGGCTGGGTTGAGCT |
| | GAGCTGGGCTGAGCTGGGCTGAGGTGAGGGCTGGGGT |
| | GAGCTGGGCTGAACTAGCCTAGCTAGGTTGGGCTGAG |
| | CTGGGCTGGTTTGGGCTGAGCTGAGCTGAGCTAGGCT |
| | GCATTGAGCAGGGTGAGCTGGGCTGAGCAGGCCTGGG |
| | GTGAGCTGGGCTAGGTGGAGCTGAGCTGGGTCGAGCT |
| | GAGTTGGGCTGAGCTGGCCTGGGTTGAGGTAGGCTGA |
| | GCTGAGCTGAGCTAGGCTGGGTTGAGCTGGCTGGGCT |
| | GGTTTGCGCTGGGTCAAGCTGGGCCGAGCTGGCCTGG |
| | GTTGAGCTGGGCTCGGTTGAGCTGGGCTGAGCTGAGC |
| | CGACCTAGGCTGGGATGAGCTGGGCTGATTTGGGCTG |
| | AGCTGAGCTGAGCTAGGCTGCATTGAGCAGGCTGAGC |
| | TGGGCCTGGAGCCTGGCCTGGGGTGAGCTGGGCTGAG |
| | CTGCGCTGAGGTAGGCTGGGTTGAGCTGGCTGGGGTG |
| | GTTTGCGCTGGGTCAAGCTGGGCCGAGCTGGCCTGGG |
| | ATGAGCTGGGCCGGTTTGGGCTGAGCTGAGCTGAGCT |
| | AGGCTGCATTGAGCAGGCTGAGCTGGGCTGAGCTGGC |
| | CTGGGGTGAGCTGGGCTGAGCTAAGCTGAGCTGGGCT |
| | GGTTTGGGCTGAGCTGGCTGAGCTGGGTCCTGCTGAG |
| | CTGGGCTGAGGTGACCAGGGGTGAGCTGGGCTGAGTT |
| | AGGCTGGGCTCAGCTAGGCTGGGTTGATCTGGCAGGG |
| | CTGGTTTGCGCTGGGTCAAGCTGCCGGGAGATGGCCT |
| | GGGATGAGGTGGGCTGGTTTGGGCTGAGCTGAGCTGA |
| | GCTGAGCTAGGGTGCATTGAGGAGGCTGAGCTGGGGT |
| | GAGCTGGCCTGGGGTGAGGTGGGCTGGGTGGAGCTGA |
| | GCTGGGCTGAACTGGGCTAAGCTGGCTGAGCTGGATC |
| | GAGCTGAGCTGGGGTGAGGTGGCCTGGGGTTAGCTGG |
| | GCTGAGCTGAGCTGAGCTAGGCTGGGTTGAGCTGGCT |
| | GGGCTGGTTTGCGCTGGGTCAAGCTGGGCGGAGCTGG |
| | CCTGGGTTGAGCTGGGCTGGGCTGAGCTGAGCTAGGC |
| | TGGGTTGAGCTGGGCTGGGCTGAGCTGAGCTAGGCTG |
| | CATTGAGCTGGCTGGGATGGATTGAGCTGGCTGAGCT |
| | GGCTGAGCTGGCTGAGCTGGGCTGAGCTGGCCTGGGT |
| | TGAGCTGGGCTGGGTTGAGCTGAGCTGGGCTGAGCTG |
| | GGCTCAGCAGAGCTGGGTTGAGCTGAGCTGGGTTGAG |
| | CTGGGGTGAGCTGGGCTGAGCAGAGCTGGGTTGAGCT |
| | GAGCTGGGTTGAGCTGGGCTCGAGCAGAGCTGGGTTG |
| | AGCTGAGCTGGGTTGAGCTGGGCTCAGCAGAGCTGGG |
| | TTGAGCTGAGCTGGGTTGAGCTGGGCTGAGCTAGCTG |
| | GGCTGAGCTAGGCTGGGCTGAGCTGAGCTGGGCTGAA |
| | CTGGGCTGAGCTGGGCTGAACTGGGCTGAGCTGGGCT |
| | GAGCTGGGCTGAGCAGAGCTGGGCTGAGCAGAGCTGG |
| | GTTGGTCTGAGCTGGGTTGAGCTGGGCTGAGCTGGGC |
| | TGAGCAGAGTTGGGTTGAGCTGAGCTGGGTTCAGCTG |
| | GGCTGAGCTAGGCTGGGTTGAGCTGGGTTGAGTTGGG |
| | CTGAGCTGGGCTGGGTTGAGCGGAGCTGGGCTGAACT |
| | GGGCTGAGCTGGGCTGAGCGGAACTGGGTTGATCTGA |
| | ATTGAGCTGGGCTGAGCCGGGCTGAGGCGGGCTGAGG |
| | TGGGGTAGGTTGAGCTTGGGTGAGCTTGCCTCAGCTG |
| | GTCTGAGCTAGGTTGGGTGGAGCTAGGCTGGATTGAG |
| | CTGGGCTGAGCTGAGCTGATCTGGCCTCAGCTGGGGT |
| | GAGGTAGGGTGAACTGGGCTGTGCTGGGCTGAGCTGA |
| | GGTGAGCCAGTTTGAGCTGGGTTGAGGTGGGCTGAGC |
| | TGGGGTGTGTTGATCTTTGCTGAACTGGGCTGAGCTG |
| | GGCTGAGCTGGCCTAGCTGGATTGAACGGGGGTAAGC |
| | TGGGCCAGGCTGGACTGGGCTGAGCTGAGCTAGGCTG |
| | AGCTGAGTTGAATTGGGTTAAGCTGGGCTGAGATGGG |
| | CTGAGCTGGGGTGAGCTGGGTTGAGCCAGGTCGGACT |
| | GGGTTAGCGTGGGCCAGACTGGGCTGAGGTGGGCGGA |
| | GCTCGattaacctggtcaggctgagtcgggtccagca |
| | gacatgcgctggccaggctggcttgacctggacacgt |
| | tcgatgagctgccttgggatggttcacctcagctgag |
| | ccaggtggctccagctgggctgagctggtgaccctgg |
| | gtgacctcggtgaccaggttgtcctgagtccgggcca |
| | agccgaggctgcatcagactcgccagacccaaggcct |
| | gggccccggctggcaagccaggggcggtgaaggctgg |
| | gctggcaggactgtcccggaaggaggtgcacgtggag |
| | ccgcccggaccccgaccggcaggacctggaaagacgc |
| | ctctcactcccctttctcttctgtcccctctcgggtc |
| | ctcagAGAGCGAGTGTGCCGGGAATCTCTACCCCCTC |
| | GTCTCCTGCGTCAGCCCCCCGTCCGATGAGAGCCTGG |
| | TGGCCCTGGGCTGCCTGGCCCGGGACTTCCTGCCCAG |
| | CTCCGTCACCTTCTCCTGGAACTACAAGAACAGCAGC |
| | AAGGTCAGCAGCCAGAACATCCAGGACTTCCCGTCCG |
| | TCCTGAGAGGCGGCAAGTACTTGGCCTCCTCCCGGGT |
| | GCTCCTACCCTCTGTGAGCATCCCCCAGGACCCAGAG |
| | GCCTTCCTGGTGTGCGAGGTGCAGCACCCCAGTGGCA |
| | CGAAGTCCGTGTCCATCTCTGGGCCAGgtgagctggg |
| | ctccccctgtggctgtggcgggggcggggccgggtgc |
| | cgccggcacagtgacgccccgttcctgcctgcagTCG |
| | TAGAGGAGCAGCCCCCCGTCTTGAACATCTTCGTGCC |
| | CAGCCGGGAGTCCTTCTCCAGTACTCCCCAGCGCACG |
| | TCCAAGCTCATCTGCCAGGCCTCAGAGTTCAGCCCCA |
| | AGGAGATGTCCATGGCCTGGTTGCGTGATGGGAAACG |
| | GGTGGTGTGTGGCGTGAGCACAGGCCCCGTGGAGACC |
| | CTACAGTCCAGTCCGGTGACCTACAGGCTCCACAGCA |
| | TGGTGACCGTCACGGAGTCCGAGTGGCTCAGCCAGAG |
| | CGTCTTCACCTGCCAGGTGGAGGACAAAGGGCTGAAG |
| | TAGGAGAAGAACGGGTCCTCTGTGTGCACCTGCAgtg |
| | agtgcagcccctcgggccgggcggcggggcggcggga |
| | gccacacacacaccagctgctccctgagccttggctt |
| | ccgggagtggccaaggcggggaggggctgtgcagggc |
| | agctggagggcactgtcagctggggcccagcaccccc |
| | tcaccccggcagggcccgggctccgaggggccccgca |
| | gtcgcaggccctgctcttgggggaagccctacttggc |
| | cccttcagggcgctgacgctccccccacccacccccg |
| | cctagATGCCAACTCTGCCATCACCGTCTTCGGCATC |
| | GCCCGCTCCTTCGCTGGCATCTTCCTCACCAAGTCGG |
| | CCAAGCTTTCCTGCCTGGTCACGGGCCTGGTCACCAG |
| | GGAGAGCCTCAACATCTCCTGGACCCGCCAGGACGGC |
| | GAGGTTCTGAAGACCAGTATCGTCTTCTCTGAGATGT |
| | ACGCCAACGGCACCTTCGGCGCCAGGGGCGAAGCCTC |
| | CGTCTGCGTGGAGGACTGGGAGTCGGGCGACAGGTTC |
| | ACGTGCACGGTGACCCACACGGACCTGCGCTGGCCGC |
| | TGAAGCAGAGCGTCTGCAAGCCCAGAGgtaggccctg |
| | ccctgcccctgcctccgcccggcctgtgccttggccg |
| | ccggggcgggagccgagcctggccgaggagcgccctc |
| | ggccccccgcggtcccgacccacacccctcctgctct |
| | cctccccagGGATCGCCAGGCACATGGCGTCCGTGTA |
| | GGTGCTGCCGCCGGCCCCGGAGGAGCTGAGCGTGCAG |
| | GAGTGGGCCTCGGTCAGCTGCCTGGTGAAGGGCTTCT |
| | CCCCGGCGGACGTGTTCGTGCAGTGGCTGCAGAAGGG |
| | GGAGCCCGTGTCCGCCGACAAGTACGTGACCAGCGCG |
| | CCGGTGCCCGAGCCCGAGCCCAAGGCCCCCGCCTCGT |
| | ACTTCGTGCAGAGCGTCCTGACGGTGAGCGCCAAGGA |
| | CTGGAGCGACGGGGAGACCTACACCTGCGTCGTGGGC |
| | CACGAGGCCCTGCCCCACACGGTGACCGAGAGGACCG |
| | TGGACAAGTCCACCGGTAAACCCACCCTGTACAACGT |
| | CTCCCTGGTCGTGTCCGACACGGCCAGCACCTGCTAC |
| | TGACCCCGTGGCTGCCCGCCGCGGCCGGGGCCAGAGC |
| | CCCCGGGCGACCATCGCTCTGTGTGGGCCTGTGTGCA |
| | ACCCGACCCTGTCGGGGTGAGCGGTCGCATTTCTGAA |
| | AATTAGAaataaaAGATCTCGTGCCG |
| Seq ID No.1 | TCTAgAAGACGCTGGAGAGAGGCCagACTTCCTCGGA |
| | ACAGCTCAAAGAGCTCTGTCAAAGCCAGATCCCATCA |
| | CACGTGGGCACCAATAGGCCATGCCAGCCTCCAAGGG |
| | CCGAACTGGGTTCTCCACGGCGCACATGAAGCCTGCA |
| | GCCTGGCTTATGCTCTTCCGTGGTGAAGAGGCAGGCC |
| | CGGGAGTGGACGAGGGGCTAGCAGGGTGTGGTAGGCA |
| | CGTTGCGGCCCCGAGCCCGGCAGGAACCAGAGACCCT |
| | GGGGCTGAGAGTGAGCCTCCAAACAGGATGCCCCACC |
| | CTTGAGGCCACCTTTCAATCCAGCTACACTCCACCTG |
| | CCATTCTCCTCTGGGCACAGGGCCCAGCCCCTGGATC |
| | TTGGCCTTGGCTCGACTTGCACCCACGCGCACACACA |
| | CACTTCCTAACGTGCTGTCCGCTCACCCCTCCCCAGC |
| | GTGGTCCATGGGCAGCACGGCAGTGGGCGTCCGGCGG |
| | TAGTGAGTGCAGAGGTCCCTTCCCCTCCCCCAGGAGC |
| | CCCAGGGGTGTGTGCAGATCTGGGGGCTCCTGTCCCT |
| | TACACCTTCATGCCCCTCCCCTCATACCCACCCTCCA |
| | GGCGGGAGGCAGCGAGACCTTTGCCCAGGGACTCAGC |
| | CAACGGGCACACGGGAGGCCAGCCCTCAGCAGCTGGG |
| Seq ID No.4 | GGCCAGACTTCCTCGGAACAGCTCAAAGAGCTCTGTC |
| | AAAGCCAGATCCCATGACAGGTGGGCACCAATAGGCC |
| | ATGCCAGCCTCCAAGGGCCGAACTGGGTTCTCCACGG |
| | CGCACATGAAGCCTGCAGCCTGGCTTATCCTCTTCCG |
| | TGGTGAAGAGGCAGGCCCGGGACTGGACGAGGGGCTA |
| | GCAGGGTGTGGTAGGCACCTTGCGCCCCCCACCCCGG |
| | CAGGAACCAGAGACCCTGGGGCTGAGAGTGAGGCTCC |
| | AAACAGGATGCCCCACCCTTCAGGCCACCTTTCAATC |
| | CAGCTACACTCCACCTGCCATTCTGCTCTGGGCACAG |
| | GGCCCAGCCCCTGGATCTTGGCGTTGGCTCGACTTGC |
| | ACCCACGCGCACACACACACTTCGTAACGTGCTGTCC |
| | GCTCACCCCTCCCCAGCGTGGTCCATGGGCAGCACGG |
| | CAGTGCGCGTCGGGCGGTAGTGAGTGCAGAGGTCCCT |
| | TCCCCTGCCCCAGGAGCCCCAGGGGTGTGTGCAGATC |
| | TGGGGGGTCCTGTCCCTTACACCTTCATGGGCCTCCC |
| | CTCATACCCACCCTCCAGGCGGGAGGCAGCGAGAGCT |
| | TTGCCCAGGGACTCAGCCAACGGGCACACGGGAGGCC |
| | AGCCCTCAGCAGCTGGCTCCCAAAGAGGAGGTGGGAG |
| | GTAGGTCCACAGCTGCCACAGAGAGAAACCCTGACGG |
| | ACCCCACAGGGGGCACGCCAGCCGGAACCAGCTCCCT |
| | CGTGGGTGAGCAATGGCCAGGGCCCCGCCGGCCACCA |
| | CGGCTGGCCTTGCGCCAGCTGAGAACTCACGTCCAGT |
| | GCAGGGAGACTCAAGACAGCCTGTGCACACAGCCTCG |
| | GATCTGCTCCCATTTCAAGCAGAAAAAGGAAACCGTG |
| | CAGGCAGCCCTCAGCATTTCAAGGATTGTAGCAGCGG |
| | CCAACTATTCGTCGGCAGTGGCCGATTAGAATGACCG |
| | TGGAGAAGGGCGGAAGGGTGTCTCGTGGGCTCTGCGG |
| | CCAACAGGCCCTGGCTCCACCTGCCCGCTGCCAGCCC |
| | GAGGGGCTTGGGCCGAGCCAGGAACCACAGTGCTCAC |
| | GGGGACCACAGTGACTGAGGAAACTCGGGGCGAGAGC |
| | AGCGCCAGGCCAGCCGGGGTCTCGCCCTGGAGGACTG |
| | ACCATCAGATGCACAAGGGGGCGAGTGTGGAAGAGAC |
| | GTGTCGCGCGGGCGATTTGGGAAGGCGAAGGGACCTT |
| | TCCAGGTGGACAGGAGGTGGGACGCACTCCAGGCAAG |
| | GGACTGGGTCCCCAAGGCCTGGGGAAGGGGTACTGGC |
| | TTGGGGGTTTAGCCTGGCCAGGGAACGGGGAGCGGGG |
| | CGGGGGGCTGAGCAGGGAGGACCTGACCTCGTGGGAG |
| | CGAGGCAAGTCAGGCTTCAGGCAGCAGCCGCACATCC |
| | CAGACCAGGAGGCTGAGGCAGGAGGGGCTTGCAGCGG |
| | GGCGGGGGCCTGCCTGGCTCCGGGGGCTCCTGGGGGA |
| | CGCTGGCTGTTGTTTCGGTGTCCCGCAGCACAGGGCC |
| | AGCTCGCTGGGCCTATGCTTACCTTGATGTCTGGGGC |
| | CGGGGCGTCAGGGTCGTCGTCTCCTCAGGGGAGAGTC |
| | CCCTGAGGCTACGCTGGGG*GGGGACTATGGCAGCTC |
| | CACCAGGGGCCTGGGGACCAGGGGCCTGGACCAGGCT |
| | GCAGCCCGGAGGACGGGCAGGGCTCTGGCTCTCCAGC |
| | ATCTGGCCCTCGGAAATGGCAGAACCCCTGGGGGGTG |
| | AGCGAGCTGAGAGCGGGTCAGACAGACAGGGGCCGGC |
| | CGGAAAGGAGAAGTTGGGGGCAGAGCCCGCCAGGGGC |
| | CAGGCCCAAGGTTCTGTGTGCCAGGGCCTGGGTGGGC |
| | ACATTGGTGTGGCCATGGCTACTTAGATTCGTGGGGG |
| | CAGGGCATCCTGGTCACCGTCTCCTCAGGTGAGGCTG |
| | GTGTCTGATGTCCAGCTAGGCGCTGGTGGGCCGCGGG |
| | TGGGCCTGTCTCAGGCTAGGGCAGGGGCTGGGATGTG |
| | TATTTGTCAAGGAGGGGCAACAGGGTGCAGACTGTGC |
| | CCCTGGAAACTTGACCACTGGGGCAGGGGCGTCCTGG |
| | TCACGTCTCCTCAGGTAAGACGGCCCTGTGCCCCTCT |
| | CTCGCGGGACTGGAAAAGGAATTTTCCAAGATTCCTT |
| | GGTCTGTGTGGGGCCCTCTGGGGCCCCCGGGGGTGGC |
| | TCCCCTCCTGCCCAGATGGGGCCTCGGCCTGTGGAGC |
| | ACGGGCTGGGCACACAGCTCGAGTCTAGGGCCACAGA |
| | GGCCCGGGCTCAGGGCTCTGTGTGGCCCGGCGACTGG |
| | CAGGGGGCTCGGGTTTTTGGACACCCCCTAATGGGGG |
| | CCACAGCACTGTGACCATCTTCACAGCTGGGGCCGAG |
| | GAGTCGAGGTCACCGTCTCCTCAGGTGAGTCCTCGTC |
| | AGCCCTCTCTCACTCTCTGGGGGGTTTTGCTGCATTT |
| | TGTGGGGGAAAGAGGATGCCTGGGTCTCAGGTCTAAA |
| | GGTCTAGGGCCAGCGCCGGGGCCCAGGAAGGGGCCGA |
| | GGGGCCACGCTCGGCTCGGCCAGGAGCAGAGCTTCCA |
| | GACATCTCGCGTCCTGGCGGCTGCAGTCAGGCCTTTG |
| | GCCGGGGGGGTCTCAGCACCACCAGGCGTCTTGGGTC |
| | CCGAGGTCCCCGGCCCCGGCTGCCTCACCAGGCACCG |
| | TGCGCGGTGGGCCCGGGCTCTTGGTCGGCCACCCTTT |
| | CTTAACTGGGATCCGGGCTTAGTTGTCGCAATGTGAC |
| | AACGGGCTCGAAAGCTGGGGCCAGGGGACCCTAGT*T |
| | ACGACGCCTCGGGTGGGTGTCGCGCACCCCTCCCCAC |
| | TTTCACGGCACTCGGCGAGACCTGGGGAGTCAGGTGT |
| | TGGGGACACTTTGGAGGTCAGGAACGGGAGCTGGGGA |
| | GAGGGCTCTGTCAGCGGGGTCCAGAGATGGGGCGCCC |
| | TCCAAGGACGCCCTGCGCGGGGACAAGGGCTTCTTGG |
| | CCTGGCCTGGCCGCTTCACTTGGGCGTCAGGGGGGGC |
| | TTCCCGGGGCAGGCGGTCAGTCGAGGCGGGTTGGAAT |
| | TCTGAGTCTGGGTTCGGGGTTCGGGGTTCGGCCTTCA |
| | TGAACAGACAGCCCAGGCGGGCCGTTGTTTGGCCCCT |
| | GGGGGCGTGGTTGGAATGCGAGGTGTCGGGAAGTCAG |
| | GAGGGAGCCTGGCCAGCAGAGGGTTCGCAGCCCTGCG |
| | GCCGAGGGACCTGGAGACGGGCAGGGCATTGGCCGTC |
| | GCAGGGCCAGGCCACACCCCCCAGGTTTTTGTGGGGC |
| | GAGCCTGGAGATTGCACCACTGTGATTACTATGCTAT |
| | GGATCTCTGGGGCCCAGGCGTTGAAGTCGTCGTGTCC |
| | TCAGGTAAGAACGGCCCTCCAGGGCCTTTAATTTCTG |
| | CTCTCGTCTGTGGGCTTTTCTGACTCTGATCCTCGGG |
| | AGGCGTCTGTGCCCCCCCCGGGGATGAGGCCGGCTTG |
| | CCAGGAGGGGTCAGGGACCAGGAGCCTGTGGGAAGTT |
| | CTGACGGGGGCTGCAGGCGGGAAGGGCCGCACCGGGG |
| | GGCGAGCCCCAGGCCGCTGGGCGGCAGGAGACCCGTG |
| | AGAGTGCGCCTTGAGGAGGGTGTCTGCGGAACCACGA |
| | ACGCCCGCCGGGAAGGGCTTGCTGCAATGCGGTCTTC |
| | AGACGGGAGGCGTCTTCTGCCCTCACCGTCTTTCAAG |
| | CCCTTGTGGGTCTGAAAGAGCCATGTCGGAGAGAGAA |
| | GGGAGAGGCCTGTCCCGACCTGGCCGAGAGCGGGCAG |
| | CCCCGGGGGAGAGCGGGGCGATCGGCCTGGGCTCTGT |
| | GAGGCCAGGTCCAAGGGAGGACGTGTGGTCCTCGTGA |
| | CAGGTGCACTTGCGAAACCTTAGAAGACGGGGTATGT |
| | TGGAAGCGGCTCCTGATGTTTAAGAAAAGGGAGACTG |
| | TAAAGTGAGCAGAGTCCTCAAGTGTGTTAAGGTTTTA |
| | AAGGTCAAAGTGTTTTAAACCTTTGTGACTGCAGTTA |
| | GCAAGCGTGCGGGGAGTGAATGGGGTGGCAGGGTGGC |
| | CGAGAGGCAGTACGAGGGCCGTGCCGTCCTCTAATTC |
| | AGGGCTTAGTTTTGCAGAATAAAGTCGGCCTGTTTTC |
| | TAAAAGCATTGGTGGTGCTGAGCTGGTGGAGGAGGCC |
| | GCGGGCAGGCCTGGCCACCTGCAGCAGGTGGCAGGAA |
| | GCAGGTCGGCCAAGAGGCTATTTTAGGAAGCCAGAAA |
| | ACACGGTCGATGAATTTATAGCTTCTGGTTTCCAGGA |
| | GGTGGTTGGGCATGGCTTTGCGCAGCGCCACAGAACC |
| | GAAAGTGCCCACTGAGAAAAAACAACTCCTGCTTAAT |
| | TTGCATTTTTCTAAAAGAAGAAACAGAGGCTGACGGA |
| | AACTGGAAAGTTCCTGTTTTAACTACTCGAATTGAGT |
| | TTTCGGTCTTAGCTTATCAACTGCTCACTTAGATTCA |
| | TTTTCAAAGTAAACGTTTAAGAGCCGAGGCATTCCTA |
| | TCCTCTTCTAAGGCGTTATTCCTGGAGGCTCATTCAC |
| | CGCCAGCACCTCCGCTGCCTGCAGGCATTGCTGTCAC |
| | CGTCACCGTGACGGCGCGCACGATTTTCAGTTGGCCC |
| | GCTTCCCCTCGTGATTAGGACAGACGCGGGCACTCTG |
| | GCCCAGCCGTCTTGGCTCAGTATCTGCAGGCGTCCGT |
| | CTCGGGACGGAGCTCAGGGGAAGAGCGTGACTCCAGT |
| | TGAACGTGATAGTCGGTGCGTTGAGAGGAGACCCAGT |
| | CGGGTGTCGAGTCAGAAGGGGCCCGGGGCCCGAGGCC |
| | CTGGGCAGGACGGCGCGTGCCCTGCATCACGGGCCCA |
| | GCGTGCTAGAGGCAGGACTCTGGTGGAGAGTGTGAGG |
| | GTGCCTGGGGCCCCTCCGGAGCTGGGGCCGTGCGGTG |
| | CAGGTTGGGCTCTCGGCGCGGTGTTGGCTGTTTCTGC |
| | GGGATTTGGAGGAATTCTTCCAGTGATGGGAGTCGCC |
| | AGTGACCGGGCACCAGGCTGGTAAGAGGGAGGCCGCC |
| | GTCGTGGCCAGAGCAGCTGGGAGGGTTCGGTAAAAGG |
| | CTCGCCCGTTTCCTTTAATGAGGACTTTTCCTGGAGG |
| | GCATTTAGTCTAGTCGGGACCGTTTTCGACTCGGGAA |
| | GAGGGATGCGGAGGAGGGCATGTGCCCAGGAGCCGAA |
| | GGCGCCGCGGGGAGAAGCCCAGGGCTCTCCTGTCCCC |
| | ACAGAGGCGACGCCACTGCCGCAGACAGACAGGGCCT |
| | TTCCCTCTGATGACGGCAAAGGCGCCTCGGCTCTTGC |
| | GGGGTGCTGGGGGGGAGTCGCCCCGAAGCCGCTCACC |
| | CAGAGGCCTGAGGGGTGAGACTGACCGATGCCTGTTG |
| | GCCGGGCCTGGGGCCGGACCGAGGGGGACTCCGTGGA |
| | GGCAGGGCGATGGTGGCTGCGGGAGGGAACCGACCCT |
| | GGGCCGAGCCCGGCTTGGCGATTCCCGGGCGAGGGCC |
| | CTCAGCCGAGGCGAGTGGGTCCGGCGGAACCACCCTT |
| | TCTGGCCAGCGCCACAGGGCTCTCGGGACTGTCCGGG |
| | GCGACGCTGGGCTGCCCGTGGCAGGCCTGGGCTGACC |
| | TGGACTTCACCAGACAGAACAGGGCTTTCAGGGCTGA |
| | GGTGAGCCAGGTTTAGCGAGGCCAAGTGGGGCTGAAC |
| | CAGGCTCAACTGGCCTGAGCTGGGTTGAGCTGGGCTG |
| | ACCTGGGCTGAGCTGAGCTGGGCTGGGCTGGGCTGGG |
| | CTGGGCTGGGCTGGGCTGGACTGGCTGAGCTGAGCTG |
| | GGTTGAGCTGAGCTGAGCTGGCCTGGGTTGAGCTGGG |
| | CTGGGTTGAGGTGAGGTGGGTTGAGCTGGGTTGAGGT |
| | GGGTTGATCTGAGCTGAGCTGGGCTGAGGTGAGCTAG |
| | GCTGGGGTGAGCTGGGCTGAGCTGGTTTGAGTTGGGT |
| | TGAGCTGAGCTGAGCTGGGCTGTGCTGGCTGAGCTAG |
| | GCTGAGCTAGGCTAGGTTGAGGTGGGCTGGGCTGAGG |
| | TGAGCTAGGCTGGGCTGATTTGGGCTGAGCTGAGCTG |
| | AGCTAGGCTGCGTTGAGCTGGCTGGGCTGGATTGAGC |
| | TGGCTGAGCTGGCTGAGCTGGGCTGAGCTGGCCTGGG |
| | TTGAGCTGAGCTGGACTGGTTTGAGCTGGGTCGATCT |
| | GGGTTGAGCTGTCCTGGGTTGAGCTGGGCTGGGTTGA |
| | GCTGAGCTGGGTTGAGCTGGGCTCAGCAGAGCTGGGT |
| | TGGGCTGAGCTGGGTTGAGCTGAGCTGGGCTGAGCTG |
| | GCCTGGGTTGAGCTGGGCTGAGCTGAGCTGGGCTGAG |
| | GTGGCCTGTGTTGAGCTGGGCTGGGTTGAGCTGGGCT |
| | GAGCTGGATTGAGCTGGGTTGAGCTGAGCTGGGCTGG |
| | GCTGTGCTGACTGAGCTGGGCTGAGCTAGGCTGGGGT |
| | GAGCTGGGCTGAGCTGATCCGAGGTAGGCTGGGCTGG |
| | TTTGGGCTGAGCTGAGCTGAGCTAGGCTGGATTGATC |
| | TGGCTGAGCTGGGTTGAGCTGAGCTGGGCTGAGCTGG |
| | TCTGAGCTGGCCTGGGTCGAGCTGAGCTGGACTGGTT |
| | TGAGCTGGGTCGATCTGGGCTGAGCTGGCCTGGGTTG |
| | AGCTGGGCTGGGTTGAGCTGAGCTGGGTTGAGGTGGG |
| | CTGAGCTGAGGGCTGGGGTGAGCTGGGCTGAACTAGC |
| | CTAGCTAGGTTGGGCTGAGCTGGGCTGGTTTGGGCTG |
| | AGCTGAGCTGAGCTAGGCTGCATTGAGCAGGCTGAGC |
| | TGGGCTGAGCAGGCCTGGGGTGAGCTGGGCTAGGTGG |
| | AGCTGAGCTGGGTCGAGCTGAGTTGGGCTGAGCTGGC |
| | CTGGGTTGAGGTAGGCTGAGCTGAGCTGAGCTAGGCT |
| | GGGTTGAGCTGGCTGGGCTGGTTTGCGCTGGGTCAAG |
| | CTGGGCCGAGCTGGCCTGGGTTGAGCTGGGCTCGGTT |
| | GAGCTGGGCTGAGCTGAGCCGACCTAGGCTGGGATGA |
| | GCTGGGCTGATTCGGGCTGAGCTGAGCTGAGCTAGGC |
| | TGCATTGAGCAGGCTGAGCTGGGCCTGGAGGCTGGCC |
| | TGGGGTGAGCTGGGCTGAGCTGCGCTGAGCTAGGCTG |
| | GGTTGAGCTGGCTGGGCTGGTTTGCGCTGGGTCAAGC |
| | TGGGCCGAGCTGGCCTGGGATGAGCTGGGCCGGTTTG |
| | GGCTGAGCTGAGCTGAGCTAGGCTGCATTGAGCAGGC |
| | TGAGCTGGGCTGAGCTGGCCTGGGGTGAGCTGGGCTG |
| | AGCTAAGCTGAGCTGGGCTGGTTTGGGCTGAGCTGGC |
| | TGAGCTGGGTCCTGCTGAGCTGGGCTGAGCTGACCAG |
| | GGGTGAGCTGGGCTGAGTTAGGCTGGGCTCAGCTAGG |
| | CTGGGTTGATCTGGCAGGGCTGGTTTGCGCTGGGTCA |
| | AGCTCCCGGGAGATGGCCTGGGATGAGCTGGGCTGGT |
| | TTGGGCTGAGCTGAGCTGAGCTGAGCTAGGCTGCATT |
| | GAGCAGGCTGAGCTGGGCTGAGCTGGCCTGGGGTGAG |
| | CTGGGCTGGGTGGAGCTGAGCTGGGCTGAACTGGGCT |
| | AAGGTGGGTGAGCTGGATCGAGCTGAGCTGGGCTGAG |
| | CTGGCGTGGGGTTAGCTGGGCTGAGCTGAGCTGAGCT |
| | AGGGTGGGTTGAGCTGGCTGGGCTGGTTTGCGCTGGG |
| | TCAAGCTGGGCCGAGCTGGCCTGGGTTGAGCTGGGGT |
| | GGGCTGAGCTGAGCTAGGCTGGGTTGAGCTGGGGTGG |
| | GCTGAGCTGAGCTAGGCTGCATTGAGCTGGCTGGGAT |
| | GGATTGAGCTGGCTGAGCTGGCTGAGCTGGCTGAGCT |
| | GGGCTGAGCTGGCCTGGGTTGAGCTGGGCTGGGTTGA |
| | GCTGAGCTGGGCTGAGCTGGGCTCAGCAGAGCTGGGT |
| | TGAGCTGAGCTGGGTTGAGCTGGGGTGAGCTGGGCTG |
| | AGCAGAGCTGGGTTGAGCTGAGCTGGGTTGAGCTGGG |
| | CTCGAGGAGAGCTGGGTTGAGCTGAGGTGGGTTGAGC |
| | TGGGCTCAGCAGAGGTGGGTTGAGCTGAGCTGGGTTG |
| | AGCTGGGGTGAGGTAGCTGGGGTCAGCTAGGGTGGGT |
| | TGAGGTGAGCTGGGCTGAACTGGGCTGAGCTGGGCTG |
| | AACTGGGCTGAGCTGGGCTGAGCTGGGCTGAGCAGAG |
| | GTGGGGTGAGCAGAGCTGGGTTGGTCTGAGCTGGGTT |
| | GAGCTGGGGTGAGCTGGGGTGAGCAGAGTTGGGTTGA |
| | GCTGAGCTGGGTTCAGCTGGGCTGAGGTAGGCTGGGT |
| | TGAGGTGGGTTGAGTTGGGCTGAGCTGGGCTGGGTTG |
| | AGCGGAGCTGGGCTGAACTGGGTTGAGGTGGGCTGAG |
| | CGGAACTGGGTTGATCTGAATTGAGCTGGGGTGAGCC |
| | GGGCTGAGGCGGGCTGAGCTGGGCTAGGTTGAGCTTG |
| | GGTGAGGTTGCCTCAGCTGGTCTGAGCTAGGTTGGGT |
| | GGAGCTAGGCTGGATTGAGCTGGGCTGAGCTGAGCTG |
| | ATCTGGCCTCAGCTGGGCTGAGGTAGGCTGAACTGGG |
| | CTGTGCTGGGCTGAGCTGAGGTGAGCCAGTTTGAGCT |
| | GGGTTGAGCTGGGCTGAGCTGGGCTGTGTTGATCTTT |
| | CCTGAACTGGGCTGAGGTGGGCTGAGGTGGCCTAGCT |
| | GGATTGAACGGGGGTAAGCTGGGCCAGGCTGGAGTGG |
| | GGTGAGCTGAGCTAGGCTGAGCTGAGTTGAATTGGGT |
| | TAAGCTGGGCTGAGATGGGGTGAGCTGGGCTGAGCTG |
| | GGTTGAGCCAGGTCGGACTGGGTTACGCTGGGCCACA |
| | CTGGGCTGAGCTGGGCGGAGCTCGATTAACGTGGTCA |
| | GGCTGAGTGGGGTCCAGCAGACATGCGCTGGCCAGGC |
| | TGGCTTGACCTGGACAGGTTGGATGAGCTGCCTTGGG |
| | ATGGTTCACCTCAGCTGAGGCAGGTGGCTCCAGCTGG |
| | GCTGAGCTGGTGACCCTGGGTGACCTCGGTGACCAGG |
| | TTGTCCTGAGTCCGGGCCAAGGCGAGGCTGCATCAGA |
| | CTCGCCAGACCCAAGGCGTGGGCCCCGGCTGGCAAGC |
| | CAGGGGCGGTGAAGGCTGGGCTGGCAGGACTGTCCCG |
| | GAAGGAGGTGCACGTGGAGCCGCCCGGACCCCGACCG |
| | GCAGGACCTGGAAAGACGCCTCTCACTCCCCTTTCTC |
| | TTCTGTCCCCTCTCGGGTCCTCAGAGAGCCAGTCTGC |
| | CCCGAATCTGTACCGCCTGGTCTCCTGCGTCAGCCCC |
| | CCGTCCGATGAGAGCCTGGTGGCCCTGGGCTGCCTGG |
| | CCCGGGACTTCCTGCCCAGCTGCGTCACCTTCTCCTG |
| | GAA |

Porcine Kappa Light Chain

In another embodiment, novel genomic sequences encoding the kappa light chain locus of ungulate immunoglobulin are provided. The present invention provides the first reported genomic sequence of ungulate kappa light chain regions. In one embodiment, nucleic acid sequence is provided that encodes the porcine kappa light chain locus. In another embodiment, the nucleic acid sequence can contain at least one joining region, one constant region and/or one enhancer region of kappa light chain. In a further embodiment, the nucleotide sequence can include at least five joining regions, one constant region and one enhancer region, for example, as represented in Seq ID No. 30. In a further embodiment, an isolated nucleotide sequence is provided that contains at least one, at least two, at least three, at least four or five joining regions and 3′ flanking sequence to the joining region of porcine genomic kappa light chain, for example, as represented in Seq ID No 12. In another embodiment, an isolated nucleotide sequence of porcine genomic kappa light chain is provided that contains 5′ flanking sequence to the first joining region, for example, as represented in Seq ID No. 25. In a further embodiment, an isolated nucleotide sequence is provided that contains 3′ flanking sequence to the constant region and, optionally, the 5′ portion of the enhancer region, of porcine genomic kappa light chain, for example, as represented in Seq ID Nos. 15, 16 and/or 19.

In further embodiments, isolated nucleotide sequences as depicted in Seq ID Nos 30, 12, 25, 15, 16 or 19 are provided. Nucleic acid sequences at least 80, 85, 90, 95, 98 or 99% homologous to Seq ID Nos 30, 12, 25, 15, 16 or 19 are also provided. In addition, nucleotide sequences that contain at least 10, 15, 17, 20, 25 or 30 contiguous nucleotides of Seq ID Nos 30, 12, 25, 15, 16 or 19 are provided. In addition, nucleotide sequences that contain at least 10, 15, 17, 20, 25 or 30 contiguous nucleotides of Seq ID Nos 1, 4 or 29 are provided. In other embodiments, nucleotide sequences that contain at least 50, 100, 1,000, 2,500, 5,000, 7,000, 8,000, 8,500, 9,000, 10,000 or 15,000 contiguous nucleotides of Seq ID No. 30 are provided. Further provided are nucleotide sequences that hybridizes, optionally under stringent conditions, to Seq ID Nos 30, 12, 25, 15, 16 or 19, as well as, nucleotides homologous thereto.

In one embodiment, an isolated nucleotide sequence encoding kappa light chain is provided that includes at least five joining regions, one constant region and one enhancer region, for example, as represented in Seq ID No. 30. In Seq ID No. 30, the coding region of kappa light chain is represented, for example by residues 1-549 and 10026-10549, whereas the intronic sequence is represented, for example, by residues 550-10025, the Joining region of kappa light chain is represented, for example, by residues 5822-7207 (for example, J1:5822-5859, J2:6180-6218, J3:6486-6523, J4:6826-6863, J5:7170-7207), the Constant Region is represented by the following residues: 10026-10549 (C exon) and 10026-10354 (C coding), 10524-10529 (Poly(A) signal) and 11160-11264 (SINE element).

| | |
|---|---|
| Seq ID No 30 | GCGTCCGAAGTCAAAAATATCTGCAGCCTTCATGTAT |
| | TCATAGAAACAAGGAATGTCTACATTTTGCAAAGTGG |
| | GAGCAGAATGTTGGGTCATGTCTAAGGCATGTGCATT |
| | TGCACATGGTAGGCAAAGGACTTTGCTTCTCCCAGCA |
| | CATCTTTCTGCAGAGATCCATGGAAACAAGACTCAAC |
| | TCGAAAGCAGCAAAGAAGCAGCAAGTTGTCAAGTGAT |
| | GTCCTGTGACTCCGTCGTCGCAGGGTAATGAAGCCAT |
| | GTTGCCCCTGGGGGATTAAGGGCAGGTGTCCATTGTG |
| | GCACCCAGCCCGAAGACAAGCAATTTGATCAGGTTGT |
| | GAGCAGTCCTGAATGTGGACTCTGGAATTTTCTCCTC |
| | ACCTTGTGGCATATCAGCTTAAGTCAAGTACAAGTGA |
| | CAAACAACATAATCCTAAGAAGAGAGGAATCAAGCTG |
| | AAGTCAAAGGATCAGTGCCTTGGATTCTACTGTGAAT |
| | GATGACCTGGAAAATATCGTGAACAACAGCTTCAGGG |
| | TGATCATCAGAGACAAAAGTTCCAGAGCCAGgtaggg |
| | aaaccctcaagccttgcaaagagcaaaatcatgccat |
| | tgggttcttaacctgctgagtgatttactatatgtta |
| | ctgtgggaggcaaagcgctcaaatagcctgggtaagt |
| | atgtcaaataaaaagcaaaagtggtgtttcttgaaat |
| | gttagacctgaggaaggaatattgataacttaccaat |
| | aattttcagaatgatttatagatgtgcacttagtcag |
| | tgtctctccaccccgcacctgacaagcagtttagaat |
| | ttattctaagaatctaggtttgctgggggctacatgg |
| | gaatcagcttcagtgaagagtttgttggaatgattca |
| | ctaaattttctatttccagcataaatccaagaacctc |
| | tcagactagtttattgacactgcttttcctccataat |
| | ccatctcatctccgtccatcatggacactttgtagaa |
| | tgacaggtcctggcagagactcacagatgcttctgaa |
| | acatcctttgccttcaaagaatgaacagcacacatac |
| | taaggatctcagtgatccacaaattagtttttgccac |
| | aatggttcttatgataaaagtctttcattaacagcaa |
| | attgttttataatagttgttctgctttataataattg |
| | catgcttcactttcttttcttttctttttttttcttt |
| | ttttgctttttagtgccgcaggtgcagcatatgaaat |
| | ttcccaggctaggggtcaaatcagaactacacctact |
| | ggcctacgccacagccacagcaactcaggatctaagc |
| | catgtcggtgacctacactacagctcatggcaatgcc |
| | agatccttaacccaatgagcgaggccagggatcgaac |
| | ccatgtcctcatggatactagtcaggctcattatccg |
| | ctgagccataacaggaactcccgagtttgctttttat |
| | caaaattggtacagccttattgtttctgaaaaccaca |
| | aaatgaatgtattcacataattttaaaaggttaaata |
| | atttatgatatacaagacaatagaaagagaaaacgtc |
| | attgcctctttcttccacgacaacacgcctccttaat |
| | tgatttgaagaaataactactgagcatggtttagtgt |
| | acttctttcagcaattagcctgtattcatagccatac |
| | atattcaattaaaatgagatcatgatatcacacaata |
| | cataccatacagcctatagggatttttacaatcatct |
| | tccacatgactacataaaaacctacctaaaaaaaaaa |
| | aaaaccctacttcatcctcctattggctgctttgtgc |
| | tccattaaaaagctctatcataattaggttatgatga |
| | ggatttccattttctacctttcaagcaacatttcaat |
| | gcacagtcttatatacacatttgagcctacttttctt |
| | tttctttctttttttggtttttttttttttttttttt |
| | ttggtctttttgtcttttctaaggctgcatatggagg |
| | ttcccaggctagctgtctaatcagaactatagctgct |
| | ggcctacgccacatccacagcaatacaagatctgagc |
| | catgtctgcaacttacaccacagctcacagcaacggt |
| | ggatccttaaaccactgagcaaggccagggatcaaac |
| | ccataacttcatggctcctagttggatttgttaacca |
| | ctgagccatgatggcaactcctgagcctacttttcta |
| | atcatttccaaccctaggacacttttttaagtttcat |
| | ttttctccccccaccccctgttttctgaagtgtgttt |
| | gcttccactgggtgacttcactcccaggatctcatct |
| | gcaggatactgcagctaagtgtatgagctctgaattt |
| | gaatcccaactctgccactcaaagggataggagtttc |
| | cgatgtggcccaatgggatcagtggcatctctgcagt |
| | gccaggacgcaggttccatccctggcccagcacagtg |
| | ggttaagaatctggcattgctgcagctgaggcataga |
| | tttcaattgtgcctcagatctgatccttggcccaagg |
| | actgcatatgcctcagggcaaccaaaaaagagaaaag |
| | gggggtgatagcattagtttctagatttgggggataa |
| | ttaaataaagtgatccatgtacaatgtatggcatttt |
| | gtaaatgctcaacaaatttcaactattatggagttcc |
| | catcatggctcagtggaagggaatctgattagcatcc |
| | atgaggacacaggtccaaccccgaccttgctcagtgg |
| | gcattgctgtgagctgtggcatgggttacagacgaag |
| | ctcggatctggcattgctgtggctgtggtgtaagcca |
| | gcaactacagctctcattcagcccctagcctgggaac |
| | ctccatatgcctaaaagacaaaaaataaaatttaaat |
| | taaaaataaagaaatgttaactattatgattggtact |
| | gcttgcattactgcaaagaaagtcactttctatactc |
| | tttaatatcttagttgactgtgtgctcagtgaactat |
| | tttggacacttaatttccactctcttctatctccaac |
| | ttgacaactctctttcctctcttctggtgagatccac |
| | tgctgactttgctctttaaggcaactagaaaagtgct |
| | cagtgacaaaatcaaagaaagttaccttaatcttcag |
| | aattacaatcttaagttctcttgtaaagcttactatt |
| | tcagtggttagtattattccttggtcccttacaactt |
| | atcagctctgatctattgctgattttcaactatttat |
| | tgttggagttttttccttttttccctgttcattctgc |
| | aaatgtttgctgagcatttgtcaagtgaagatactgg |
| | actgggccttccaaatataagacaatgaaacatcggg |
| | agttctcattatggtgcagcagaaacgaatccaacta |
| | ggaaatgtgaggttgcaggttcgatccctgcccttgc |
| | tcagtgggttaaggatccagcattaccgtgagctgtg |
| | gtgtaggttgcagacgtggctcagatcctgcgttgct |
| | gtggctgtggcataggctggcagctctagctctgatt |
| | cgaccgctagcctgggaacctccatgcgccccgagtg |
| | cagcccttaaaaagcaaaaaaaaaagaaagaaagaaa |
| | aagacaatgaaacatcaaacagctaacaatccagtag |
| | ggtagaaagaatctggcaacagataagagcgattaaa |
| | tgttctaggtccagtgaccttgcctctgtgctctaca |
| | cagtcgtgccacttgctgagggagaaggtctctcttg |
| | agttgagtcctgaaagacattagttgttcacaaacta |
| | atgccagtgagtgaaggtgtttccaagcagagggaga |
| | gtttggtaaaaagctggaagtcacagaaagactctaa |
| | agagtttaggatggtgggagcaacatacgctgagatg |
| | gggctggaaggttaagagggaaacaactatagtaagt |
| | gaagctggactcacagcaaagtgaggacctcagcatc |
| | cttgatggggttaccatggaaacaccaaggcacacct |
| | tgatttccaaaacagcaggcacctgattcagcccaat |
| | gtgacatggtgggtacccctctagctctacctgttct |
| | gtgacaactgacaaccaacgaagttaagtctggattt |
| | tctactctgctgatccttgtttttgtttcacacgtca |
| | tctatagcttcatgccaaaatagagttcaaggtaaga |
| | cgcgggccttggtttgatatacatgtagtctatcttg |
| | tttgagacaatatggtggcaaggaagaggttcaaaca |
| | ggaaaatactctctaattatgattaactgagaaaagc |
| | taaagagtcccataatgacactgaatgaagttcatca |
| | tttgcaaaagccttcccccccccccaggagactataa |
| | aaaagtgcaattttttaaatgaacttatttacaaaac |
| | agaaatagactcacagacataggaaacgaacagatgg |
| | ttaccaagggtgaaagggagtaggagggataaataag |
| | gagtctggggttagcagatacaccccagtgtacacaa |
| | aataaacaacagggacctactatatagcacagggaac |
| | tatatgcagtagcttacaataacctataatggaaaag |
| | aatgtgaaaaagaatatatgtatgcgtgtgtgtgtaa |
| | ctgaatcactttgctgtaacctgaatctaacataaca |
| | ttgtaaatcaactacagttttttttttttttaagtgc |
| | agggttttggtgttttttttttttcatttttgttttt |
| | gtttttgttttttgctttttagggccacacccagaca |
| | tatgggggttcccaggctaggggtctaattagagcta |
| | cagttgccggcttgcaccacagccacagcaacatcag |
| | atccgagccgcacttgcgacttacaccacagctcatg |
| | gcaataccagatccttaacccactgagcaaggcccag |
| | ggatcgtacccgcaacctcatggttcctagtcagatt |
| | catttctgctgcgctacaatgggaactccaagtgcag |
| | ttttttgtaatgtgcttgtctttctttgtaattcata |
| | ttcatcctacttcccaataaataaataaatacataaa |
| | taataaacataccattgtaaatcaactacaatttttt |
| | ttaaatgcagggtttttgttttttgttttttgttttg |
| | tctttttgccttttctagggccgctcccatggcatat |
| | ggaggttcccaggctaggggtcgaatcggagctgtag |
| | ccaccggcctacgccagagccacagcaacgcgggatc |
| | cgagccgcgtctgcaacctacaccacagctcacggca |
| | acgccggatcgttaacccactgagcaagggcagggat |
| | cgaacctgcaacctcatggttcctagtcagattcgtt |
| | aactactgagccacaacggaaactcctaaagtgcagt |
| | ttttaaatgtgcttgtctttctttgtaatttacactc |
| | aacctacttcccaataaataaataaataaacaaataa |
| | atcatagacatggttgaattctaaaggaagggaccat |
| | caggccttagacagaaatacgtcatcttctagtattt |
| | taaaacacactaaagaagacaaacatgctctgccaga |
| | gaagcccagggcctccacagctgcttgcaaagggagt |
| | taggcttcagtagctgacccaaggctctgttcctctt |
| | cagggaaaagggtttttgttcagtgagacagcagaca |
| | gctgtcactgtgGTGGACGTTCGGCCAAGGAACCAAG |
| | CTGGAAGTCAAACgtaagtcaatccaaacgttccttc |
| | cttggctgtctgtgtcttacggtctctgtggctctga |
| | aatgattcatgtgctgactctctgaaaccagactgac |
| | attctccagggcaaaactaaagcctgtcatcaaactg |
| | gaaaactgagggcacattttctgggcagaactaagag |
| | tcaggcactgggtgaggaaaaacttgttagaatgata |
| | gtttcagaaacttactgggaagcaaagcccatgttct |
| | gaacagagctctgctcaagggtcaggaggggaaccag |
| | tttttgtacaggagggaagttgagacgaacccctgtg |
| | TATATGGTTTCGGCGCGGGGACCAAGCTGGAGCTGAA |
| | ACgtaagtggctttttccgactgattctttgctgttt |
| | ctaattgttggttggctttttgtccatttttcagtgt |
| | tttcatcgaattagttgtcagggaccaaacaaattgc |
| | cttcccagattaggtaccagggaggggacattgctgc |
| | atgggagaccagagggtggctaatttttaacgtttcc |
| | aagccaaaataactggggaagggggcttgctgtcctg |
| | tgagggtaggtttttatagaagtggaagttaagggga |
| | aatcgctatgGTTCACTTTTGGCTCGGGGACCAAAGT |
| | GGAGCCCAAAAttgagtacattttccatcaattattt |
| | gtgagatttttgtcctgttgtgtcatttgtgcaagtt |
| | tttgacattttggttgaatgagccattcccagggacc |
| | caaaaggatgagaccgaaaagtagaaaagagccaact |
| | tttaagctgagcagacagaccgaattgttgagtttgt |
| | gaggagagtagggtttgtagggagaaaggggaacaga |
| | tcgctggctttttctctgaattagcctttctcatggg |
| | actggcttcagagggggtttttgatgagggaagtgtt |
| | ctagagccttaactgtgGGTTGTGTTGGGTAGCGGCA |
| | CCAAGCTGGAAATCAAACgtaagtgcacttttctact |
| | cctttttctttcttatacgggtgtgaaattggggact |
| | tttcatgtttggagtatgagttgaggtcagttctgaa |
| | gagagtgggactcatccaaaaatctgaggagtaaggg |
| | tcagaacagagttgtctcatggaagaacaaagaccta |
| | gttagttgatgaggcagctaaatgagtcagttgactt |
| | gggatccaaatggccagacttcgtctgtaaccaacaa |
| | tctaatgagatgtagcagcaaaaagagatttccattg |
| | aggggaaagtaaaattgttaatattgtgGATCACCTT |
| | TGGTGAAGGGACATCCGTGGAGATTGAACgtaagtat |
| | tttttctctactaccttctgaaatttgtctaaatgcc |
| | agtgttgacttttagaggcttaagtgtcagttttgtg |
| | aaaaatgggtaaacaagagcatttcatatttattatc |
| | agtttcaaaagttaaactcagctccaaaaatgaattt |
| | gtagacaaaaagattaatttaagccaaattgaatgat |
| | tcaaaggaaaaaaaaattagtgtagatgaaaaaggaa |
| | ttcttacagctccaaagagcaaaagcgaattaatttt |
| | ctttgaactttgccaaatcttgtaaatgatttttgtt |
| | ctttacaatttaaaaaggttagagaaatgtatttctt |
| | agtctgttttctctcttctgtctgataaattattata |
| | tgagataaaaatgaaaattaataggatgtgctaaaaa |
| | atcagtaagaagttagaaaaatatatgtttatgttaa |
| | agttgccacttaattgagaatcagaagcaatgttatt |
| | tttaaagtctaaaatgagagataaactgtcaatactt |
| | aaattctgcagagattctatatcttgacagatatctc |
| | ctttttcaaaaatccaatttctatggtagactaaatt |
| | tgaaatgatcttcctcataatggagggaaaagatgga |
| | ctgaccccaaaagctcagatttaaagaaatctgttta |
| | agtgaaagaaaataaaagaactgcattttttaaaggc |
| | ccatgaatttgtagaaaaataggaaatattttaataa |
| | gtgtattcttttattttcctgttattacttgatggtg |
| | tttttataccgccaaggaggccgtggcaccgtcagtg |
| | tgatctgtagaccccatggcggccttttttcgcgatt |
| | gaatgaccttggcggtgggtccccagggctctggtgg |
| | cagcgcaccagccgctaaaagccgctaaaaactgccg |
| | ctaaaggccacagcaaccccgcgaccgcccgttcaac |
| | tgtgctgacacagtgatacagataatgtcgctaacag |
| | aggagaatagaaatatgacgggcacacgctaatgtgg |
| | ggaaaagagggagaagcctgatttttattttttagag |
| | attctagagataaaattcccagtattatatcctttta |
| | ataaaaaatttctattaggagattataaagaatttaa |
| | agctatttttttaagtggggtgtaattctttcagtag |
| | tctcttgtcaaatggatttaagtaatagaggcttaat |
| | ccaaatgagagaaatagacgcataaccctttcaaggc |
| | aaaagctacaagagcaaaaattgaacacagcagccag |
| | ccatctagccactcagattttgatcagttttactgag |
| | tttgaagtaaatatcatgaaggtataattgctgataa |
| | aaaaataagatacaggtgtgacacatctttaagtttc |
| | agaaatttaatggcttcagtaggattatatttcacgt |
| | atacaaagtatctaagcagataaaaatgccattaatg |
| | gaaacttaatagaaatatatttttaaattccttcatt |
| | ctgtgacagaaattttctaatctgggtcttttaatca |
| | cctaccctttgaaagagtttagtaatttgctatttgc |
| | catcgctgtttactccagctaatttcaaaagtgatac |
| | ttgagaaagattatttttggtttgcaaccacctggca |
| | ggactattttagggccattttaaaactcttttcaaac |
| | taagtattttaaactgttctaaaccatttagggcctt |
| | ttaaaaatcttttcatgaatttcaaacttcgttaaaa |
| | gttattaaggtgtctggcaagaacttccttatcaaat |
| | atgctaatagtttaatctgttaatgcaggatataaaa |
| | ttaaagtgatcaaggcttgacccaaacaggagtatct |
| | tcatagcatatttcccctcctttttttctagaattca |
| | tatgattttgctgccaaggctattttatataatctct |
| | ggaaaaaaaatagtaatgaaggttaaaagagaagaaa |
| | atatcagaacattaagaattcggtattttactaactg |
| | cttggttaacatgaaggtttttattttattaaggttt |
| | ctatctttataaaaatctgttcccttttctgctgatt |
| | tctccaagcaaaagattcttgatttgttttttaactc |
| | ttactctcccacccaagggcctgaatgcccacaaagg |
| | ggacttccaggaggccatctggcagctgctcaccgtc |
| | agaagtgaagccagccagttcctcctgggcaggtggc |
| | caaaattacagttgacccctcctggtctggctgaacc |
| | ttgccccatatggtgacagccatctggccagggccca |
| | ggtctccctctgaagcctttgggaggagagggagagt |
| | ggctggcccgatcacagatgcggaaggggctgactcc |
| | tcaaccggggtgcagactctgcagggtgggtctgggc |
| | ccaacacacccaaagcacgcccaggaaggaaaggcag |
| | cttggtatcactgcccagagctaggagaggcaccggg |
| | aaaatgatctgtccaagacccgttcttgcttctaaac |
| | tccgagggggtcagatgaagtggttttgtttcttggc |
| | ctgaagcatcgtgttccctgcaagaagcggggaacac |
| | agaggaaggagagaaaagatgaactgaacaaagcatg |
| | caaggcaaaaaaggccttaggatggctgcaggaagtt |
| | agttcttctgcattggctccttactggctcgtcgatc |
| | gcccacaaacaacgcacccagtggagaacttccctgt |
| | tacttaaacaccattctctgtgcttgcttcctcagGG |
| | GGTGATGCCAAGCCATCGGTCTTCATCTTCCCGCCAT |
| | CGAAGGAGCAGTTAGCGACCCCAACTGTCTCTGTGGT |
| | GTGCTTGATCAATAACTTCTTCCCCAGAGAAATCAGT |
| | GTCAAGTGGAAAGTGGATGGGGTGGTCCAAAGCAGTG |
| | GTCATCCGGATAGTGTCACAGAGCAGGACAGCAAGGA |
| | CAGCACGTAGAGGGTCAGCAGCAGGGTCTCGCTGCCC |
| | ACGTCACAGTACCTAAGTCATAATTTATATTCCTGTG |
| | AGGTCAGCCACAAGACCCTGGCGTCCCCTCTGGTCAC |
| | AAGCTTCAACAGGAACGAGTGTGAGGCTtagAGGCCG |
| | ACAGGCCCCTGGCCTGCCCCCAGGGCCAGCCCGCCTC |
| | CGCACCTCAAGCCTCAGGCCCTTGCCCCAGAGGATCG |
| | TTGGCAATCCCCCAGCCCCTCTTGCCTCCTCATCCCC |
| | TCGCCCTCTTTGGCTTTAAGCGTGTTAATAGTGGGGG |
| | GTGGGGGAATGAATAaataaaGTGAACGTTTGGACCT |
| | GTGAtttctctctcctgtctgattttaaggttgttaa |
| | atgttgttttccccattatagttaatcttttaaggaa |
| | ctacatactgagttgctaaaaactacaccatcactta |
| | taaaattcacgccttctcagttctcccctcccctcct |
| | gtcctccgtaagacaggcctccgtgaaacccataagc |
| | acttctctttacaccctctcctgggccggggtaggag |
| | actttttgatgtcccctcttcagcaagcctcagaacc |
| | attttgagggggacagttcttacagtcacat*tcctg |
| | tgatctaatgactttagttaccgaaaagccagtctct |
| | caaaaagaagggaacggctagaaaccaagtcatagaa |
| | atatatatgtataaaatatatatatatccatatatgt |
| | aaaataacaaaataatgataacagcataggtcaacag |
| | gcaacagggaatgttgaagtccattctggcacttcaa |
| | tttaagggaataggatgccttcattacattttaaata |
| | caatacacatggagagcttcctatctgccaaagacca |
| | tcctgaatgccttccacactcactacaaggttaaaag |
| | cattcattacaatgttgatcgaggagttcccgttgtg |
| | gctcagcaggttaagaacgtgactggtatccaggagg |
| | atgcgggtttggtccccagcctcgctcagtggattaa |
| | ggatccagtgttgctgcaagatcacgggctcagatcc |
| | cgtgttctatggctatggtgtaggctggtagctgcat |
| | gcagccctaatttgacccctagcctgggaactgccat |
| | atgccacatgtgaggcccttaaaacctaaaagaaaaa |
| | aaaagaaaagaaatatcttacacccaatttatagata |
| | agagagaagctaaggtggcaggcccaggatcaaagcc |
| | ctacctgcctatcttgacacctgatacaaattctgtc |
| | ttctagggtttccaacactgcatagaacagagggtca |
| | aacatgctaccctcccagggactcctcccttcaaatg |
| | acataaattttgttgcccatctctgggggcaaaactc |
| | aacaatcaatggcatctctagtaccaagcaaggctct |
| | tctcatgaagcaaaactctgaagccagatccatcatg |
| | acccaaggaagtaaagacaggtgttactggttgaact |
| | gtatccttcaattcaatatgctcaatttccaactccc |
| | agtccccgtaaatacaaccccctttgggaagagagtc |
| | cttgcagatgtagccacgttaaaaagagattatacag |
| | aaaggctagtgaggatgcagtgaaacgggatctttca |
| | tacattgctggtggaaatgtaaaatgctgcaggcact |
| | ctagaaaataatttgccagttttttgaaaagctaaac |
| | aaaatagtttagttgcattctgggttatttatccccc |
| | agaaattaaaaattatgtccgcacaaaaacgtgtaca |
| | taatcattcataacagccttgtac |
| Seq ID No.12 | caaggaaccaagctggaactcaaacgtaagtcaatcc |
| | aaacgttccttccttggctgtctgtgtcttacggtct |
| | ctgtggctctgaaatgattcatgtgctgactctctga |
| | aaccagactgacattctccagggcaaaactaaagcct |
| | gtcatcaaactggaaaactgagggcacattttctggg |
| | cagaactaagagtcaggcactgggtgaggaaaaactt |
| | gttagaatgatagtttcagaaacttactgggaagcaa |
| | agcccatgttctgaacagagctctgctcaagggtcag |
| | gaggggaaccagtttttgtacaggagggaagttgaga |
| | cgaacccctgtgtatatggtttcggcgcggggaccaa |
| | gctggagctcaaacgtaagtggctttttccgactgat |
| | tctttgctgtttctaattgttggttggctttttgtcc |
| | atttttcagtgttttcatcgaattagttgtcagggac |
| | caaacaaattgccttcccagattaggtaccagggagg |
| | ggacattgctgcatgggagaccagagggtggctaatt |
| | tttaacgtttccaagccaaaataactggggaaggggg |
| | cttgctgtcctgtgagggtaggtttttatagaagtgg |
| | aagttaaggggaaatcgctatggttcacttttggctc |
| | ggggaccaaagtggagcccaaaattgagtacattttc |
| | catcaattatttgtgagatttttgtcctgttgtgtca |
| | tttgtgcaagtttttgacattttggttgaatgagcca |
| | ttcccagggacccaaaaggatgagaccgaaaagtaga |
| | aaagagccaacttttaagctgagcagacagaccgaat |
| | tgttgagtttgtgaggagagtagggtttgtagggaga |
| | aaggggaacagatcgctggctttttctctgaattagc |
| | ctttctcatgggactggcttcagagggggtttttgat |
| | gagggaagtgttctagagccttaactgtgggttgtgt |
| | tcggtagcgggaccaagctggaaatcaaacgtaagtg |
| | cacttttctactcctttttctttcttatacgggtgtg |
| | aaattggggacttttcatgtttggagtatgagttgag |
| | gtcagttctgaagagagtgggactcatccaaaaatct |
| | gaggagtaagggtcagaacagagttgtctcatggaag |
| | aacaaagacctagttagttgatgaggcagctaaatga |
| | gtcagttgacttgggatccaaatggccagacttcgtc |
| | tgtaaccaacaatctaatgagatgtagcagcaaaaag |
| | agatttccattgaggggaaagtaaaattgttaatatt |
| | gtggatcacctttggtgaagggacatccgtggagatt |
| | gaacgtaagtattttttctctactaccttctgaaatt |
| | tgtctaaatgccagtgttgacttttagaggcttaagt |
| | gtcagttttgtgaaaaatgggtaaacaagagcatttc |
| | atatttattatcagtttcaaaagttaaactcagctcc |
| | aaaaatgaatttgtagacaaaaagattaatttaagcc |
| | aaattgaatgattcaaaggaaaaaaaaattagtgtag |
| | atgaaaaaggaattcttacagctccaaagagcaaaag |
| | cgaattaattttctttgaactttgccaaatcttgtaa |
| | atgatttttgttctttacaatttaaaaaggttagaga |
| | aatgtatttcttagtctgttttctctcttctgtctga |
| | taaattattatatgagataaaaatgaaaattaatagg |
| | atgtgctaaaaaatcagtaagaagttagaaaaatata |
| | tgtttatgttaaagttgccacttaattgagaatcaga |
| | agcaatgttatttttaaagtctaaaatgagagataaa |
| | ctgtcaatacttaaattctgcagagattctatatctt |
| | gacagatatctcctttttcaaaaatccaatttctatg |
| | gtagactaaatttgaaatgatcttcctcataatggag |
| | ggaaaagatggactgaccccaaaagctcagattt*aa |
| | gaaaacctgtttaag*gaaagaaaataaaagaactgc |
| | attttttaaaggcccatgaatttgtagaaaaatagga |
| | aatattttaataagtgtattcttttattttcctgtta |
| | ttacttgatggtgtttttataccgccaaggaggccgt |
| | ggcaccgtcagtgtgatctgtagaccccatggcggcc |
| | ttttttcgcgattgaatgaccttggcggtgggtcccc |
| | agggctctggtggcagcgcaccagccgctaaaagccg |
| | ctaaaaactgccgctaaaggccacagcaaccccgcga |
| | ccgcccgttcaactgtgctgacacagtgatacagata |
| | atgtcgctaacagaggagaatagaaatatgacgggca |
| | cacgctaatgtggggaaaagagggagaagcctgattt |
| | ttattttttagagattctagagataaaattcccagta |
| | ttatatccttttaataaaaaatttctattaggagatt |
| | ataaagaatttaaagctatttttttaagtggggtgta |
| | attctttcagtagtctcttgtcaaatggatttaagta |
| | atagaggcttaatccaaatgagagaaatagacgcata |
| | accctttcaaggcaaaagctacaagagcaaaaattga |
| | acacagcagccagccatctagccactcagattttgat |
| | cagttttactgagtttgaagtaaatatcatgaaggta |
| | taattgctgataaaaaaataagatacaggtgtgacac |
| | atctttaagtttcagaaatttaatggcttcagtagga |
| | ttatatttcacgtatacaaagtatctaagcagataaa |
| | aatgccattaatggaaacttaatagaaatatattttt |
| | aaattccttcattctgtgacagaaattttctaatctg |
| | ggtcttttaatcacctaccctttgaaagagtttagta |
| | atttgctatttgccatcgctgtttactccagctaatt |
| | tcaaaagtgatacttgagaaagattatttttggtttg |
| | caaccacctggcaggactattttagggccattttaaa |
| | actcttttcaaactaagtattttaaactgttctaaac |
| | catttagggccttttaaaaatcttttcatgaatttca |
| | aacttcgttaaaagttattaaggtgtctggcaagaac |
| | ttccttatcaaatatgctaatagtttaatctgttaat |
| | gcaggatataaaattaaagtgatcaaggcttgaccca |
| | aacaggagtatcttcatagcatatttcccctcctttt |
| | tttctagaattcatatgattttgctgccaaggctatt |
| | ttatataatctctggaaaaaaaatagtaatgaaggtt |
| | aaaagagaagaaaatatcagaacattaagaattcggt |
| | attttactaactgcttggttaacatgaaggtttttat |
| | tttattaaggtttctatctttataaaaatctgttccc |
| | ttttctgctgatttctccaagcaaaagattcttgatt |
| | tgttttttaactcttactctcccacccaagggcctga |
| | atgcccacaaaggggacttccaggaggccatctggca |
| | gctgctcaccgtcagaagtgaagccagccagttcctc |
| | ctgggcaggtggccaaaattacagttgacccctcctg |
| | gtctggctgaaccttgccccatatggtgacagccatc |
| | tggccagggcccaggtctccctctgaagcctttggga |
| | ggagagggagagtggctggcccgatcacagatgcgga |
| | aggggctgactcctcaaccggggtgcagactctgcag |
| | ggtgggtctgggcccaacacacccaaagcacgcccag |
| | gaaggaaaggcagcttggtatcactgcccagagctag |
| | gagaggcaccgggaaaatgatctgtccaagacccgtt |
| | cttgcttctaaactccgagggggtcagatgaagtggt |
| | tttgtttcttggcctgaagcatcgtgttccctgcaag |
| | aagcggggaacacagaggaaggagagaaaagatgaac |
| | tgaacaaagcatgcaaggcaaaaaaggccttaggatg |
| | gctgcaggaagttagttcttctgcattggctccttac |
| | tggctcgtcgatcgcccacaaacaacgcacccagtgg |
| | agaacttccctgttacttaaacaccattctctgtgct |
| | tgcttcctcaggggctgatgccaagccatccgtcttc |
| | atcttcccgccatcgaaggagcagttagcgaccccaa |
| | ctgtctctgtggtgtgcttgatca |
| Seq ID No.15 | gatgccaagccatccgtcttcatcttcccgccatcga |
| | aggagcagttagcgaccccaactgtctctgtggtgtg |
| | cttgatcaataacttcttccccagagaaatcagtgtc |
| | aagtggaaagtggatggggtggtccaaagcagtggtc |
| | atccggatagtgtcacagagcaggacagcaaggacag |
| | cacctacagcctcagcagcaccctctcgctgcccacg |
| | tcacagtacctaagtcataatttatattcctgtgagg |
| | tcacccacaagaccctggcctcccctctggtcacAAG |
| | CTTCAACAGGAACGAGTGTGAGGCTTAGAGGCCCACA |
| | GGGGCCTGGCGTGCCCCGAGCCCCAGCCCCGCTCCCC |
| | ACCTCAAGCCTCAGGCCCTTGCCCCAGAGGATCCTTG |
| | GCAATCCCCCAGCCCCTCTTCCGTCGTCATGCGGTCC |
| | CCCTGTTTGGCTTTAACCGTGTTAATACTGGGGGGTG |
| | GGGGAATGAATAAATAAAGTGAACCTTTGCACCTGTG |
| | ATTTCTCTCTCCTGTCTGATTTTAAGGTTGTTAAATG |
| | TTGTTTTCCCCATTATAGTTAATCTTTTAAGGAACTA |
| | CATACTGAGTTGCTAAAAACTACACCATCACTTATAA |
| | AATTCAcgCCTTCTCAGTTCTCCCCTCCCCTCCTGTC |
| | CTCCGTAAGACAGGCCTCCGTGAAACCCATAAGCACT |
| | TCTCTTTACACCCTCTCCTGGGCCGGGGTAGGAGACT |
| | TTTTGATGTCCCCTcTTCAGCAAGCCTCAGAACCATT |
| | TTGAGGGGGACAGTTCTTACAGTCACAT*TCCtGtGA |
| | TCTAATGACTTTAGTTaCCGAAAAGCCAGTCTCTCAA |
| | AAAGAAGGGAACGGCTAGAAACCAAGTCATAGAAATA |
| | TATATGTATAAAATATATATATATCCATATATGTAAA |
| | ATAACAAAATAATGATAAGAGCATAGGTCAACAGGCA |
| | ACAGGGAATGTTGAAGTCGATTCTGGCAGTTCAATTT |
| | AAGGGAATAGGATGCCTTCATTACATTTTAAATAGAA |
| | TACACATGGAGAGCTTCGTATCTGCCAAAGACCATCC |
| | TGAATGCCTTCCACACTCACTACAAGGTTAAAAGCAT |
| | TCATTACAATGTTGATCGAGGAGTTCCCGTTGTGGCT |
| | CAGCAGGTTTAAGAAGGTGACTGGTATGCAGGAGGAT |
| | GCGGGTTGGTCCCGAGCCTCGCTCAGTGGATTAAGGA |
| | TCCAGTGTTGCTGCAAGATCACGGGCTCAGATCCCGT |
| | GTTCTATGGCTATGGTGTAGGCTGGTAGCTGCATGCA |
| | GCCCTAATTTGACCCCTAGCCTGGGAACTGCCATAtG |
| | CCACATGTGAGGCCCTTAAAACGTAAAAGAAAAAaAA |
| | AGAAAAGAAATATCTTACACGCAATTTATAGATAAGA |
| | GAGAAGCTAAGGTGGCAGGCCCAGGATGAAAGCCCTA |
| | CGTGCCTATCTTGACACCTGAtACAAATTCTGTCTTC |
| | TAGGGtTTCCAACACTGCATAGAACAGAGGGTCAAAC |
| | ATGCTACCCTCCCAGGGACTCCTCCCTTCAAATGACA |
| | TAAATTTTGTTGGCCATCTCTGGGGGCAAAACTGAAC |
| | AATCAATGGCATGTCTAGTACCAAGCAAGGCTCTTCT |
| | CATGAAGCAAAAGTGTGAAGCCAGATCCATCATGACC |
| | CAAGGAAGTAAAGACAGGTGTTACTGGTTGAACTGTA |
| | TCCTTGAATTGAATATGCTGAATTTCGAACTGCCAGT |
| | CCCCGTAAATACAACCCCCTTTGGGAAGAGAGTCCTT |
| | GCAGATGTAGCCACGTTAAAAAGAGATTATACAGAAA |
| | GGCTAGTGAGGATGCAGTGAAACGGGATCTTTCATAC |
| | ATTGCTGGTGGAAATGTAAAATGCTGCAGGCACTCTA |
| | GAAAATAATTTGCCAGTTTTTTGAAAAGCTAAACAAA |
| | ATAGTTTAGTTGCATTCTGGGTTATTTATCCCCCAGA |
| | AATTAAAAATTATGTCCGCACAAAAACGTGTACATAA |
| | TCATTCATAACAGCCTTGTACGAAAAGCTT |
| Seq ID No.16 | GGATCCTTAACCCACTAATCGAGGATCAAACACGCAT |
| | CCTCATGGACAATATGTTGGGTTCTTAGCCTGCTGAG |
| | ACACAACAGGAACTCCCCTGGCACCACTTTAGAGGCC |
| | AGAGAAACAGCACAGATAAAATTCCCTGCCCTCATGA |
| | AGCTTATAGTGTAGCTGGGGAGATATGATAGGCAAGA |
| | TAAACACATACAAATACATGATCTTAGGTAATAATAT |
| | ATAGTAAGGAGAAAATTACAGGGGAGAAAGAGGACAG |
| | GAATTGCTAGGGTAGGATTATAAGTTCAGATAGTTCA |
| | TGAGGAACACTGTTGCTGAGAAGATAACATTTAGGTA |
| | AAGACCGAAGTAGTAAGGAAATGGACCGTGTGCCTAA |
| | GTGGGTAAGACCATTCTAGGCAGCAGGAACAGCGATG |
| | AAAGCACTGAGGTGGGTGTTCACTGCACAGAGTTGTT |
| | CACTGCACAGAGTTGTGTGGGGAGGGGTAGGTCTTGC |
| | AGGCTCTTATGGTCACAGGAAGAATTGTTTTACTCCC |
| | ACCGAGATGAAGGTTGGTGGATTTTGAGCAGAAGAAT |
| | AATTCTGCCTGGTTTATATATAACAGGATTTCCCTGG |
| | GTGCTCTGATGAGAATAATCTGTCAGGGGTGGGATAG |
| | GGAGAGATATGGCAATAGGAGCGTTGGCTAGGAGCCC |
| | ACGACAATAATTCCAAGTGAGAGGTGGTGCTGCATTG |
| | AAAGCAGGACTAACAAGACCTGCTGACAGTGTGGATG |
| | TAGAAAAAGATAGAGGAGACGAAGGTGCATCTAGGGT |
| | TTTCTGCCTGAGGAATTAGAAAGATAAAGCTAAAGCT |
| | TATAGAAGATGCAGCGCTCTGGGGAGAAAGACCAGCA |
| | GCTCAGTTTTGATCCATCTGGAATTAATTTTGGCATA |
| | AAGTATGAGGTATGTGGGTTAACATTATTTGTTTTTT |
| | TTTTTTCCATGTAGCTATCCAACTGTCCCAGCATCAT |
| | TTATTTTAAAAGACTTTCCTTTCCCCTATTGGATTGT |
| | TTTGGCACCTTCACTGAAGATCAACTGAGCATAAAAT |
| | TGGGTCTATTTCTAAGCTCTTGATTCCATTCCATGAC |
| | CTATTTGTTCATCTTTACCCCAGTAGACACTGCCTTG |
| | ATGATTAAAGCCCCTGTTACCATGTCTGTTTTGGACA |
| | TGGTAAATCTGAGATGCCTATTAGCCAACCAAGCAAG |
| | CACGGCCCTTAGAGAGCTAGATATGAGAGCCTGGAAT |
| | TCAGACGAGAAAGGTCAGTCCTAGAGACATACATGTA |
| | GTGCCATCACCATGCGGATGGTGTTAAAAGCCATCAG |
| | ACTGCAACAGAGTGTGAGAGGGTACCAAGCTAGAGAG |
| | CATGGATAGAGAAACCCAAGCACTGAGCTGGGAGGTG |
| | CTCCTACATTAAGAGATTAGTGAGATGAAGGACTGAG |
| | AAGATTGATCAGAGAAGAAGGAaAATCAGGAAAATGG |
| | TGCTGTCcTGAAAATCCAAGGGAAGAGATGTTCCAAA |
| | GAGGAGAaAACTGATGAGTTGTCAGCTAGCGTCAATT |
| | GGGATGAAAATGGACCATTGGACAGAGGGATGTAGTG |
| | GGTCATGGGTGAATAGATAAGAGCAGCTTCTATAGAA |
| | TGGCAGGGGCAAAATTCTCATCTGATCGGCATGGGTT |
| | CTAAAGAAAACGGGAAGAAAAAATTGAGTGCATGACC |
| | AGTCCCTTCAAGTAGAGAGGTgGAAAAGGGAAGGAGG |
| | AAAATGAGGCCACGACAACATGAGAGAAATGACAGCA |
| | TTTTTAAAAATTTTTTATTTTATTTtATTTATTTATT |
| | TTTGCTTTTTAGGGCTGCCCCTGCAAcatatggaggt |
| | tcccaggttaggggtctaatcagagctatagctgcca |
| | gcctacaccacagccatagcaatgccagatctacatg |
| | acctacaccacagctcacagcaacgccggatccttaa |
| | cccactgagtgaggccagagatcaaacccatatcctt |
| | atggatactagtcaggttcattaccactgagccaaaa |
| | tgggaaATCCTGAGTAATGACAGCATTTTTTAATGTG |
| | CCAGGAAGCAAAACTTGCCACCCCGAAATGTCTCTCA |
| | GGCATGTGGATTATTTTGAGCTGAAAACGATTAAGGC |
| | CCAAAAAAGACAAGAAGAAATGTGGACCTTCCGGCAA |
| | CAGCCTAAAAAATTTAGATTGAGGGCCTGTTCCCAGA |
| | ATAGAGCTATTGCCAGACTTGTCTACAGAGGCTAAGG |
| | GCTAGGTGTGGTGGGGAAACCCTCAGAGATCAGAGGG |
| | ACGTTTATGTACCAAGCATTGACATTTCCATCTCCAT |
| | GCGAATGGCCTTCTTCCCCTCTGTAGCCCCAAACCAC |
| | CACCCCCAAAATCTTCTTCTGTCTTTAGCTGAAGATG |
| | GTGTTGAAGGTGATAGTTTCAGCCACTTTGGCGAGTT |
| | CCTCAGTTGTTCTGGGTCTTTCCTCCGGATCCACATT |
| | ATTCGACTGTGTTTGATTTTCTCCTGTTTATCTGTCT |
| | CATTGGCACCCATTTCATTCTTAGACCAGCCCAAAGA |
| | ACCTAGAAGAGTGAAGGAAAATTTCTTCCACCCTGAC |
| | AAATGCTAAATGAGAATCACCgCAGTAGAGGAAAATG |
| | ATCTGGTgCTGCGGGAGATAGAAGAGAAAATcGCTGG |
| | AGAGATGTCACTGAGTAGGTGAGATGGGAAAGGGGGG |
| | GCACAGGTGGAGGTGTTGCCCTCAGCTAGGAAGACAG |
| | ACAGTTcacagaagagaagcgggtgtccgtGGACATC |
| | TTGGGTCATGGATGAGGAAACCGAGGGTAAGAAAGAG |
| | TGCAAAAGAAAGGTAAGGATTGCAGAGAGGTCGATCC |
| | ATGAGTAAAATCACAGTAACCAACGCCAAACCAGCAT |
| | GTTTTCTCCTAGTCTGGCACGTGGCAGGTACTGTGTA |
| | GGTTTTCAATATTATTGGTTTGTAACAGTACCTATTA |
| | GGCCTCCATCcCCTCCTCTAATACTAACAAAAGTGTG |
| | AGACTGGTCAGTGAAAAATGGTCTTCTTTCTCTATGC |
| | AATCTTTCTCAAGAAGATACATAACTTTTTATTTTAT |
| | CATaGGCTTGAAGAGCAAATGAGAAACAgCCTCCAAC |
| | CTATGACACCGTAACAAAGTGTTTATGATCAGTGAAG |
| | GGCAAGAAACAAAACATACACaGTAAAGACCCTCCAT |
| | AATATTGtGGGCTGGCCCAaCACAGGCCAGGTTGTAA |
| | AAGCTTTTTATTCTTTGATAGAGGAATGGATAGTAAT |
| | GTTTCAACCTGGACAGAGAT*CATGTTCACTGAATCC |
| | TTCCAAAAATTCATGGGTAGTTTGAAtTATAAGGAAA |
| | ATAAGACTTAGGATAAATACTTTgTCCA*GATCCCAG |
| | AGTTAATgCCAAAATCAGTTTTCAGACTCCAGGCAGC |
| | CTGATCAAGAGCCTAAACTTTAAAGACACAGTCCCTT |
| | AATAACTACTATTCACAGTTGCACTTTCAgGGCGCAA |
| | AGACTCATTGAATCCTACAATAGAATGAGTTTAGATA |
| | TCAAATCTCTCAGTAATAGATGAGGAGACTAAATAGC |
| | GGGCATGACCTGGTCACTTAAAGACAGAATTGAGATT |
| | CAAGGCTAGTGTTCTTTCTACCTGTTTTGTTTCTACA |
| | AGATGTAGCAATGCGCTAATTACAGACCTCTCAGGGA |
| | AGGAATTCACAACCCTCAGCAAAAACCAAAGACAAAT |
| | CTAAGACAACTAAGAGTGTTGGTTTAATTTGGAAAAA |
| | TAACTCACTAACCAAACGCCCGTCTTAGCACGCCAAT |
| | GTCTTCCACCATCACAGTGCTCAGGCCTCAACCATGC |
| | CGCAATGACCCGAGCCCCAGACTGGTTATTACCAAGT |
| | TTTCATGATGACTGGCGTGAGAAGATCAAAAAGGAAT |
| | GACATCTTACAGGGGACTACGCCGAGGACCAAGATAG |
| | CAACTGTCATAGCAACCGTCACAGTGCTTTGGTGA |
| Seq ID No.19 | ggatcaaacacgcatcctcatggacaatatgttgggt |
| | tcttagcctgctgagacacaacaggaactcccctggc |
| | accactttagaggccagagaaacagcacagataaaat |
| | tccctgccctcatgaagcttatagtctagctggggag |
| | atatcataggcaagataaacacatacaaatacatcat |
| | cttaggtaataatatatactaaggagaaaattacagg |
| | ggagaaagaggacaggaattgctagggtaggattata |
| | agttcagatagttcatcaggaacactgttgctgagaa |
| | gataacatttaggtaaagaccgaagtagtaaggaaat |
| | ggaccgtgtgcctaagtgggtaagaccattctaggca |
| | gcaggaacagcgatgaaagcactgaggtgggtgttca |
| | ctgcacagagttgttcactgcacagagttgtgtgggg |
| | aggggtaggtcttgcaggctcttatggtcacaggaag |
| | aattgttttactcccaccgagatgaaggttggtggat |
| | tttgagcagaagaataattctgcctggtttatatata |
| | acaggatttccctgggtgctctgatgagaataatctg |
| | tcaggggtgggatagggagagatatggcaataggagc |
| | cttggctaggagcccacgacaataattccaagtgaga |
| | ggtggtgctgcattgaaagcaggactaacaagacctg |
| | ctgacagtgtggatgtagaaaaagatagaggagacga |
| | aggtgcatctagggttttctgcctgaggaattagaaa |
| | gataaagctaaagcttatagaagatgcagcgctctgg |
| | ggagaaagaccagcagctcagttttgatccatctgga |
| | attaattttggcataaagtatgaggtatgtgggttaa |
| | cattatttgttttttttttttccatgtagctatccaa |
| | ctgtcccagcatcatttattttaaaagactttccttt |
| | cccctattggattgttttggcaccttcactgaagatc |
| | aactgagcataaaattgggtctatttctaagctcttg |
| | attccattccatgacctatttgttcatctttacccca |
| | gtagacactgccttgatgattaaagcccctgttacca |
| | tgtctgttttggacatggtaaatctgagatgcctatt |
| | agccaaccaagcaagcacggcccttagagagctagat |
| | atgagagcctggaattcagacgagaaaggtcagtcct |
| | agagacatacatgtagtgccatcaccatgcggatggt |
| | gttaaaagccatcagactgcaacagactgtgagaggg |
| | taccaagctagagagcatggatagagaaacccaagca |
| | ctgagctgggaggtgctcctacattaagagattagtg |
| | agatgaaggactgagaagattgatcagagaagaagga |
| | aaatcaggaaaatggtgctgtcctgaaaatccaaggg |
| | aagagatgttccaaagaggagaaaactgatcagttgt |
| | cagctagcgtcaattgggatgaaaatggaccattgga |
| | cagagggatgtagtgggtcatgggtgaatagataaga |
| | gcagcttctatagaatggcaggggcaaaattctcatc |
| | tgatcggcatgggttctaaagaaaacgggaagaaaaa |
| | attgagtgcatgaccagtcccttcaagtagagaggtg |
| | gaaaagggaaggaggaaaatgaggccacgacaacatg |
| | agagaaatgacagcatttttaaaaattttttatttta |
| | ttttatttatttatttttgctttttagggctgcccct |
| | gcaacatatggaggttcccaggttaggggtctaatca |
| | gagctatagctgccagcctacaccacagccatagcaa |
| | tgccagatctacatgacctacaccacagctcacagca |
| | acgccggatccttaacccactgagtgaggccagagat |
| | caaacccatatccttatggatactagtcaggttcatt |
| | accactgagccaaaatgggaaatcctgagtaatgaca |
| | gcattttttaatgtgccaggaagcaaaacttgccacc |
| | ccgaaatgtctctcaggcatgtggattattttgagct |
| | gaaaacgattaaggcccaaaaaacacaagaagaaatg |
| | tggaccttcccccaacagcctaaaaaatttagattga |
| | gggcctgttcccagaatagagctattgccagacttgt |
| | ctacagaggctaagggctaggtgtggtggggaaaccc |
| | tcagagatcagagggacgtttatgtaccaagcattga |
| | catttccatctccatgcgaatggccttcttcccctct |
| | gtagccccaaaccaccacccccaaaatcttcttctgt |
| | ctttagctgaagatggtgttgaaggtgatagtttcag |
| | ccactttggcgagttcctcagttgttctgggtctttc |
| | ctccTgatccacattattcgactgtgtttgattttct |
| | cctgtttatctgtctcattggcacccatttcattctt |
| | agaccagcccaaagaacctagaagagtgaaggaaaat |
| | ttcttccaccctgacaaatgctaaatgagaatcaccg |
| | cagtagaggaaaatgatctggtgctgcgggagataga |
| | agagaaaatcgctggagagatgtcactgagtaggtga |
| | gatgggaaaggggtgacacaggtggaggtgttgccct |
| | cagctaggaagacagacagttcacagaagagaagcgg |
| | gtgtccgtggacatcttgcctcatggatgaggaaacc |
| | gaggctaagaaagactgcaaaagaaaggtaaggattg |
| | cagagaggtcgatccatgactaaaatcacagtaacca |
| | accccaaaccaccatgttttctcctagtctggcacgt |
| | ggcaggtactgtgtaggttttcaatattattggtttg |
| | taacagtacctattaggcctccatcccctcctctaat |
| | actaacaaaagtgtgagactggtcagtgaaaaatggt |
| | cttctttctctatgaatctttctcaagaagatacata |
| | actttttattttatcataggcttgaagagcaaatgag |
| | aaacagcctccaacctatgacaccgtaacaaaatgtt |
| | tatgatcagtgaagggcaagaaacaaaacatacacag |
| | taaagaccctccataatattgtgggtggcccaacaca |
| | ggccaggttgtaaaagctttttattctttgatagagg |
| | aatggatagtaatgtttcaacctggacagagatcatg |
| | ttcactgaatccttccaaaaattcatgggtagtttga |
| | attataaggaaaataagacttaggataaatactttgt |
| | ccaagatcccagagttaatgccaaaatcagttttcag |
| | actccaggcagcctgatcaagagcctaaactttaaag |
| | acacagtcccttaataactactattcacagttgcact |
| | ttcagggcgcaaagactcattgaatcctacaatagaa |
| | tgagtttagatatcaaatctctcagtaatagatgagg |
| | agactaaatagcgggcatgacctggtcacttaaagac |
| | agaattgagattcaaggctagtgttctttctacctgt |
| | tttgtttctacaagatgtagcaatgcgctaattacag |
| | acctctcagggaaggaattcacaaccctcagcaaaaa |
| | ccaaagacaaatctaagacaactaagagtgttggttt |
| | aatttggaaaaataactcactaaccaaacgcccctct |
| | tagcaccccaatgtcttccaccatcacagtgctcagg |
| | cctcaaccatgccccaatcacc |
| Seq ID No.25 | GCACATGGTAGGCAAAGGACTTTGCTTCTCCCAGCAC |
| | ATCTTTCTGCAGAGATCCATGGAAACAAGACTCAACT |
| | CCAAAGCAGCAAAGAAGCAGCAAGTTCTCAAGTGATC |
| | TCCTCTGACTCCCTCCTCCCAGGCTAATGAAGCCATG |
| | TTGCCCCTGGGGGATTAAGGGCAGGTGTCCATTGTGG |
| | CACCCAGCCCGAAGACAAGCAATTTGATCAGGTTCTG |
| | AGCACTCCTGAATGTGGACTCTGGAATTTTCTCCTGA |
| | GCTTGTGGCATATCAGGTTAAGTGAAGTACAAGTGAC |
| | AAACAACATAATCGTAAGAAGAGAGGAATCAAGCTGA |
| | AGTCAAAGGATCACTGCCTTGGATTCTACTGTGAATG |
| | ATGACCTGGAAAATATCCTGAACAACAGCTTGAGGGT |
| | GATCATCAGAGACAAAAGTTCCAGAGCCAGGTAGGGA |
| | AACCGTCAAGCCTTGCAAAGAGCAAAATGATGCCATT |
| | GGGTTCTTAACCTGCTGAGTGATTTAGTATATGTTAC |
| | TGTGGGAGGCAAAGCGCTCAAATAGGGTGGGTAAGTA |
| | TGTCAAATAAAAAGCAAAAGTGGTGTTTCTTGAAATG |
| | TTAGAGCTGAGGAAGGAATATTGATAACTTACCAATA |
| | ATTTTCAGAATGATTTATAGATGTGCACTTAGTGAGT |
| | GTCTCTCCACCCCGCACCTGAGAAGCAGTTTAGAATT |
| | TATTCTAAGAATCTAGGTTTGCTGGGGGCTACATGGG |
| | AATCAGCTTCAGTGAAGAGTTTGTTGGAATGATTCAC |
| | TAAATTTTCTATTTCCAGCATAAATCCAAGAACCTCT |
| | CAGACTAGTTTATTGACACTGCTTTTCCTCCATAATC |
| | CATCTCATCTCCGTCCATCATGGACACTTTGTAGAAT |
| | GACAGGTCGTGGCAgAGACTCaCAGATGCTTCTGAAA |
| | CATCCTTTGCCTTCAAAGAATGAACAGCACACATACT |
| | AAGGATCTCAGTGATCCACAAATTAGTTTTTGCCACA |
| | ATGGTTCTTATGATAAAAGTCTTTCATTAACAGCAAA |
| | TTGTTTTATAATAGTTGTTCTGCTTTATAATAATTGC |
| | ATGCTTCACTTTCTTTTCTTTTCTTTTTTTTTCTTTT |
| | TTTGCTTTTTAGTGCCGCAGGTgcagcatatgaaatt |
| | tcccaggctaggggtcaaatcagaactacacctactg |
| | gcctacgccacagccacagcaactcaggatctaagcc |
| | atgtcggtgacctacactacagctcatggcaatgcca |
| | gatccttaacccaatgagcgaggccagggatcgaacc |
| | catgtcctcatggatactagtcaggctcattatccgc |
| | tgagccataacaggaactcccGAGTTTGCTTTTTATC |
| | AAAATTGGTACAGCCTTATTGTTTCTGAAAACCACAA |
| | AATGAATGTATTCACATAATTTTAAAAGGTTAAATAA |
| | TTTATGATATACAAGACAATAGAAAGAGAAAACGTCA |
| | TTGCCTCTTTCTTCCACGACAACACGCCTCCTTAATT |
| | GATTTGAAGAAATAACTACTGAGCATGGTTTAGTGTA |
| | CTTCTTTCAGCAATTAGCCTGTATTCATAGCCATACA |
| | TATTCAATTAAAATGAGATCATGATATCACACAATAC |
| | ATACCATACAGCCTATAGGGATTTTTACAATCATCTT |
| | CCACATGACTACATAAAAACCTACCTAAAAAAAAAAA |
| | AAACCCTACTTCATCGTCCTATTGGCTGCTTTGTGCT |
| | CCATTAAAAAGCTCTATCATAATTAGGTTATGATGAG |
| | GATTTCCATTTTCTACCTTTCAAGCAACATTTCAATG |
| | CACAGTCTTATATACACATTTGAGCCTACTTTTCTTT |
| | TTCTTTCTTTTTTTGGTTTTTTTTTTTTTTTTTTTTT |
| | TGGTCTTTTTGTCTTTTCTAAGgctgcatatggaggt |
| | tcccaggctagctgtctaatcagaactatagctgctg |
| | gcctacgccacatccacagcaatacaagatctgagcc |
| | atgtctgcaacttacaccacagctcacagcaacggtg |
| | gatccttaaaccactgagcaaggccagggatcaaacc |
| | catAACTTCATGGCTCCTAGTTGGATTTGTTAACCAC |
| | TGAGCCATGATGGCAACTCCTGAGCCTACTTTTCTAA |
| | TCATTTCCAACCCTAGGACACTTTTTTAAGTTTCATT |
| | TTTCTCCCCCCACCCCCTGTTTTCTGAAGtGTGTTTG |
| | CTTCCACTGGGTGACTTCACtCCCAGGATCTCATCTG |
| | CAGGATAGTGCAGCTAAGTGTATGAGCTCTGAATTTG |
| | AATGCGAACTCTGCGACTCAAAGGGATAGGAGTTTCC |
| | GATGTGGCGGAATGGGATCAGTGGCATGTCTGCAGTG |
| | CCAGGACGCaggttccatccctggcccagcacagtgg |
| | gttaagaatctggCATTGCTGCAGCTGAGGCATAGAT |
| | TTCAATTGTGCCTCAgATCTGATCCTTGGCCCAAGGA |
| | CTGCATATGCGTGAGGGCAACCAAAAAAGAGAAAAGG |
| | GGGGTGATAGCATTAGTTTCTAGATTTGGGGGATAAT |
| | TAAATAAAGTGATCCATGTACAATGTATGGCATTTTG |
| | TAAATGCTCAACAAATTTCAACTATTATggagttccc |
| | atcatggctcagtggaagggaatctgattagcatcca |
| | tgaggacacaggtCCAACCCCGACCTTGCTCAGTGGG |
| | CATTGCTGTGAGCTGTGGCATGGGTTACAGACGAAGC |
| | TCGGATCTGGCATTGCTGTGGCTGTGGTGTAAGCCAg |
| | CAActacagctctcattcagcccctagcctgggaacc |
| | tccatatgccTAAAAGACAAAAAATAAAATTTAAATT |
| | AAAAATAAAGAAATGTTAACTATTATGATTGgTACTG |
| | CTTGCATTACTGCAAAGAAAGTCACTTTCTATACTGT |
| | TTAATATCTTAGTTGACTGTGTGCTGAGTGAACTATT |
| | TTGGACACTTAATTTCCACTCTCTTCTATCTCCAACT |
| | TGACAACTCTCTTTCCTCTCTTCTGGTGAGATCCACT |
| | GCTGACTTTGCTCTTTAAGGCAAGTAGAAAAGTGCTC |
| | AGTGAGAAAATCAAAGAAAGTTAGCTTAATCTTCAGA |
| | ATTACAATCTTAAGTTCTCTTGTAAAGCTTACTATTT |
| | CAGTGGTTAGTATTATTCCTTGGTCCCTTACAACTTA |
| | TCAGCTCTGATCTATTGCTGATTTTCAACTATTTATT |
| | GTTGGAGTTTTTTCCTTTTTTCCCTGTTCATTCTGCA |
| | AATGTTTGCTGAGCATTTGTCAAGTGAAGATACTGGA |
| | CTGGGCCTTCCAAATATAAGACAATGAAACATGGGGA |
| | GTTCTCATTATGGTGCAGCAGAaacgaatccaactag |
| | gaaatgtgaggttgcaggttcgatccctgcccttgct |
| | cagtgggttaaggatccagcattaccgtgagctgtgg |
| | tgtaggttgcagacgtggctcagatcctgcgttgctg |
| | tggctgtggcataggctggcagctctagctctgattc |
| | gaccgctagcctgggaacctccatGCGCCCGGAGTGC |
| | AGCCCTTAAAAAGCAAAAAAAAAAGAAAGAAAGAAAA |
| | AGACAATGAAACATCAAACAGCTAACAATCGAGTAGG |
| | GTAGAAAGAATGTGGCAACAGATAAGAGCGATTAAAT |
| | GTTCTAGGTCCAGTGACCTTGCCTCTGTGCTCTACAC |
| | AGTCGTGCCACTTGCTGAGGGAGAAGGTCTCTCTTGA |
| | GTTGAGTCCTGAAAGACATTAGTTGTTCACAAACTAA |
| | TGCCAGTGAGTGAAGGTGTTTCCAAGCAGAGGGAGAG |
| | TTTGGTAAAAAGCTGGAAGTCACAGAAAGAGTCTAAA |
| | GAGTTTAGGATGGTGGGAGCAACATACGCTGAGATGG |
| | GGCTGGAAGGTTAAGAGGGAAACAACTATAGTAAGTG |
| | AAGGTGGACTCACAGCAAAGTGAGGACCTCAGCATCC |
| | TTGATGGGGTTAGCATGGAAACACCAAGGCACACCTT |
| | GATTTCCAAAACAGCAGGCACCTGATTCAGCGGAATG |
| | TGACATGGTGGGTACCCCTCTAGCTCTACCTGTTCTG |
| | TGACAACTGACAACCAACGAAGTTAAGTCTGGATTTT |
| | CTACTCTGCTGATCCTTGTTTTTGTTTCACACGTCAT |
| | CTATAGCTTCATGCCAAAATAGAGTTCAAGGTAAGAC |
| | GCGGGCCTTGGTTTGATATACATGTAGTCTATCTTGT |
| | TTGAGACAATATGGTGGCAAGGAAGAGGTTCAAACAG |
| | GAAAATACTCTCTAATTATGATTAACTGAGAAAAGCT |
| | AAAGAGTCCCATAATGACACTGAATGAAGTTCATCAT |
| | TTGCAAAAGCCTTCCCCCCCCCCCAGGAGACTATAAA |
| | AAAGTGCAATTTTTTAAATGAACTTATTTACAAAACA |
| | GAAATAGAGTCACAGACATAGGAAACGAACAGATGGT |
| | TACCAAGGGTGAAAGGGAGTAGGAGGGATAAATAAGG |
| | AGTCTGGGGTTAGCAGATACACCCCAGTGTACACAAA |
| | ATAAACAACAGGGACCTACTATATAGCACAGGGAACT |
| | ATATGCAGTAGCTTACAATAACCTATAATGGAAAAGA |
| | ATGTGAAAAAGAATATATGTATGCGTGTGTGTGTAAC |
| | TGAATCACTTTGGTGTAACCTGAATCTAACATAACAT |
| | TGTAAATCAACTACAGTTTTTTTTTTTTTTAAGTGCA |
| | GGGTTTTGGTGTTTTTTTTTTTTCATTTTTGTTTTTG |
| | TTTTTGTTTTTTGCTTTTTAGGGCCACACCCAGACAT |
| | ATGGGGGTTCCCAGGctAGGGGTcTAaTTAGAGcTAC |
| | AGtTGCCGGCTTGCAccacagccacagcaacatcaga |
| | tccgagccgcacttgcgacttacaccacagctcatgg |
| | caataccagatccttaacccactgagcaaggcccagg |
| | gatcgtacccgcaacctcatggttcctagtcagattc |
| | attTCTGCTGCGCTACAATGGGAACTCCAAGTGCAGT |
| | TTTTTGTAATGTGCTtGTCTTTCTTTGTAATTCATAT |
| | TCATCCTACTTCCCAATAAATAAATAAATACATAAAT |
| | AATAAACATACCATTGTAAATCAACTACAATTTTTTT |
| | TAAATGCAGGGTTTTTGTTTTTTGTTTTTTGTTTTGT |
| | CTTTTTGCCTTTTGTAgggccgctcccatggcatatg |
| | gaggttcccaggctaggggtcgaatcggagctgtagc |
| | caccggcctacgccagagccacagcaacgcgggatcc |
| | gagccgcgtctgcaacctacaccacagctcacggcaa |
| | cgccggatcgttaacccactgagcaagggcagggatc |
| | gaacctgcaacctcatggttcctagtcagattcgtta |
| | actactgagccacaacggaaacTCCTAAAGTGCAGTT |
| | TTTAAATGTGCTTGTCTTTCTTTGTAATTTACACTCA |
| | ACCTACTTCCCAATAAATAAATAAATAAACAAATAAA |
| | TCATAGACATGGTTGAATTCTAAAGGAAGGGACCATG |
| | AGGGCTTAGACAGAAATACGTCATGTTCTAGTATTTT |
| | AAAACACACTAAAGAAGACAAACATGCTCTGCCAGAG |
| | AAGGCCAGGGCCTCCACAGCTGCTTGCAAAGGGAGTT |
| | AGGCTTCAGTAGGTGACCCAAGGCTGTGTTGCTCTTC |
| | AGGGAAAAGGGTTTTTGTTCAGTGAGACAGCAGAGAG |
| | CTGTCACTGTGgtggacgttcggccaaggaaccaagc |
| | tggaactcaaacGTAAGTCAATCCAAACGTTCCTTCC |
| | TTGGCTGTCTGTGTCTTACGGTCTCTGTGCTCTGCTC |
| | AAGGGTCAGGAGGGGAACCAGTTTTTGTACAGGAGGG |
| | AAGTCCAGGGGAAAACTAAAGGCTGTCATCAAACcGG |
| | AAAAGTGAGGGCACATTTTCTGGGCAGAACTAAGAGT |
| | CAGGCACTGGGTGAGGAAAAACTTGTTAGAATGATAG |
| | TTTCAGAAACTTACTGGGAAGCAAAGCCCATGTTCTG |
| | AACAGAGCTCTGCTCAAGGGTCAGGAGGGGAACCAGT |
| | TTTTGTACAGGAGGGAAGTTGAGACGAACCCCTGTGT |
| | Atatggtttcggcgcggggaccaagctggagctcaaa |
| | cGTAAGTGGCTTTTTCCGACTGATTCTTTGCTGTTTC |
| | TAATTGTTGGTTGGCTTTTTGTCCATTTTTCAGTGTT |
| | TTCATCGAATTAGTTGTCAGGGACCAAACAAATTGCC |
| | TTCCCAGATTAGGTAGGAGGGAGGGGACATTGCTGCA |
| | TGGGAGACCAGAGGGTGGCTAATTTTTAACGTTTCCA |
| | AGCCAAAATAACTGGGGAAGGGGGGTTGCTGTCCTGT |
| | GAGGGTAGGTTTTTATAGAAGTGGAAGTTAAGGGGAA |
| | ATCGCTATGGTtcacttttggctcggggaccaaagtg |
| | gagcccaaaattgaGTACATTTTCCATCAATTATTTG |
| | TGAGATTTTTGTCCTGTTGTGTCATTTGTGCAAGTTT |
| | TTGACATTTTGGTTGAATGAGCCATTCCCAGGGACCC |
| | AAAAGGATGAGACCGAAAAGTAGAAAAGAGGCAACTT |
| | TTAAGCTGAGCAGACAGACCGAATTGTTGAGTTTGTG |
| | AGGAGAGTAGGGTTTGTAGGGAGAAAGGGGAACAGAT |
| | CGCTGGCTTTTTCTCTGAATTAGCCTTTCTCATGGGA |
| | CTGGCTTCAGAGGGGGTTTTTGATGAGGGAAGTGTTC |
| | TAGAGGCTTAAGTGTGGgttgtgttcggtagcgggac |
| | caagctggaaatcaaaCGTAAGTGCACTTTTCTACTC |
| | C |

Porcine Lambda Light Chain

In another embodiment, novel genomic sequences encoding the lambda light chain locus of ungulate immunoglobulin are provided. The present invention provides the first reported genomic sequence of ungulate lambda light chain regions. In one embodiment, the porcine lambda light chain nucleotides include a concatamer of J to C units. In a specific embodiment, an isolated porcine lambda nucleotide sequence is provided, such as that depicted in Seq ID No. 28.

In one embodiment, nucleotide sequence is provided that includes 5′ flanking sequence to the first lambda J/C region of the porcine lambda light chain genomic sequence, for example, as represented by Seq ID No 32. Still further, nucleotide sequence is provided that includes 3′ flanking sequence to the J/C cluster region of the porcine lambda light chain genomic sequence, for example, approximately 200 base pairs downstream of lambda J/C, such as that represented by Seq ID No 33. Alternatively, nucleotide sequence is provided that includes 3′ flanking sequence to the J/C cluster region of the porcine lambda light chain genomic sequence, for example, approximately 11.8 kb downstream of the J/C cluster, near the enhancer (such as that represented by Seq ID No. 34), approximately 12 Kb downstream of lambda, including the enhancer region (such as that represented by Seq ID No. 35), approximately 17.6 Kb downstream of lambda (such as that represented by Seq ID No. 36, approximately 19.1 Kb downstream of lambda (such as that represented by Seq ID No. 37), approximately 21.3 Kb downstream of lambda (such as that represented by Seq ID No.38), and/or approximately 27 Kb downstream of lambda (such as that represented by Seq ID No.39).

In still further embodiments, isolated nucleotide sequences as depicted in Seq ID Nos 28, 31, 32, 33, 34, 35, 36, 37, 38, or 39 are provided. Nucleic acid sequences at least 80, 85, 90, 95, 98 or 99% homologous to Seq ID Nos 28, 31, 32, 33, 34, 35, 36, 37, 38, or 39 are also provided. In addition, nucleotide sequences that contain at least 10, 15, 17, 20, 25, 30, 40, 50, 75, 100, 150, 200, 250, 500 or 1,000 contiguous nucleotides of Seq ID Nos 28, 31, 32, 33, 34, 35, 36, 37, 38, or 39are provided. Further provided are nucleotide sequences that hybridizes, optionally under stringent conditions, to Seq ID Nos 28, 31, 32, 33, 34, 35, 36, 37, 38, or 39, as well as, nucleotides homologous thereto.

| | |
|---|---|
| Seq ID No.28 | CCTTCCTCCTGCACCTGTCAACTCCCAATAAACCGTC |
| | CTCCTTGTCATTCAGAAATCATGCTCTCCGCTCACTT |
| | GTGTCTACCCATTTTCGGGCTTGCATGGGGTCATCCT |
| | CGAAGGTGGAGAGAGTCCCCCTTGGCCTTGGGGAAGT |
| | CGAGGGGGGCGGGGGGAGGCCTGAGGCATGTGCCAGC |
| | GAGGGGGGTCACCTCCACGCCCCTGAGGACCTTCTAG |
| | AACCAGGGGCGTGGGGCCACGGCCTGAGTGGAAGGGT |
| | GTCGACTTTTCCCCCGGGCGCCCAGGCTCCCTCCTCC |
| | GTGTGGACCTTGTCCACCTCTGACTGGCCCAGCCACT |
| | CATGCATTGTTTCCCCGAAACCCCAGGACGATAGCTC |
| | AGCACGCGACAGTGTCCCCCTCTGAGGGCCTCTGTCC |
| | ATTTCAGGAGGACCCGCATGTACAGGGTGACCACTCT |
| | GGTCAGGCCCACTCACCACGTCCTAGAGCCCCACCCC |
| | CAGCCCCATCCTTAGGGGCACAGCCAGcTCCGACCGC |
| | CCCGGGGACACCACCCTCTGCCCCTTcCCCAGGCGCT |
| | CCCTGTCACACGCACCACAGGGCCCTCCGTCCCGAGA |
| | CCCTGCTCCCTCATCCCTCGGTCGCCTCAGGTAGCCT |
| | TCCACCGGCGTGTGTCCCGAGGTCCCAGATGCAGGAA |
| | GGCCCCTGGGACAACGCCAGATGTCTGCTCTcCCCGA |
| | CCCCTCAGAAGGGAGCCCACGCGTGGCCCCACCAGCA |
| | CTGCCTAACgTCCAAGTGTCCATAGGCCTCGGGACCT |
| | CCAAGTCCAGGTTCTGCCTCTGGGATTCGGCCATGGG |
| | TCTGCCTGGGAAATGATGGACTTGGAGGAGCTCAGGA |
| | TGGGATGCGGGACGTTGTCTCTAGGCGCTcCCTCAGG |
| | ATCCCACAGCTGCCCTGTGAGACACACACACACACAC |
| | ACACACACACACACACACACACACAGACACAAACACG |
| | CATGCACGCACGCCGGCACAGACGGTATTGCAGAGAT |
| | GGCCACGGTAGCTGTGCCTCGAGGCCGAGTGGAGTGT |
| | CTAGAACTCTCGGGGGTCGGCTGTGCAGACGACACTG |
| | CTCCATCCCCCCCGTGCCCTGAAGGGCTCCTCAGTCT |
| | CCCATCAGGATCTCTCCAAGGTGCTGAGCTGGAGAGG |
| | AAGGGGCCTGGGACAGGCGGGGACACTCAGACCTCGC |
| | TGCTGCCCCTCCTGTGCCTGGGCTTGGACGGCTCCCC |
| | CCTTCCCACGGGTGAAGGTGCAGGTGGGGAGAGGGCA |
| | CCCCGGTCAGCCTCCCAGAGGCAGAGCAGGGCCCGTG |
| | GCAGGGGCAGCCTGTGAGGCTCGAGCCAGATGGAGGT |
| | GGCCTGGGGTGGGGGGTGGAGGGGGCGGGAGGTTTAT |
| | GTTTGAGGCTGTATCACTGTGTAATATTTTCGGCGGT |
| | GGGACCCATCTGACCGTCCTCGGTGAGTCTCCCCTTT |
| | TCTCTCCTCCTTGGGGATCCGAGTGAAATCTGGGTCG |
| | ATCTTCTCTCCGTTCTCCTCGGACTGGGGGTGAGGTC |
| | TGAAGCTCGGTGGGGTCCGAAGAGGAGGCCCCTAGGC |
| | GAGGCTGCTCAGCCCCTCCAGCGCGAGcgGCCGTGTT |
| | GACACAGGGTCCAGCTAAGGGGAGACATGGAGGCTGC |
| | TAGTCCAGGGCCAGGCTCTGAGACCCAAGGGCGCTGC |
| | CCAAGGAACCCTTGCCCCAGGGACCCTGGGAGCAAAG |
| | GTGCTCACTCAGAGCCTGCAGCGGTGGGGTCTGAGGA |
| | CAAGGAGGGACTGAGGACTGGGCGTGGGGAGTTCAGG |
| | CGGGGACACCAGGTCCAGGGAGGTGACAAAGGCGCTG |
| | GGAGGGGGCGGACGGTGCCGGGGAGTCCTCCTGGGCC |
| | CTGTGGGCTCGGGGTCCTTGTGAGGACCCTGAGGGAC |
| | TGAGGGGCCCCTGGGCCTAGGGACTTGCAgTgAGGGA |
| | GGCAGGGAGTGTCCCTTGAGAACGTGGCCTCCGCGGG |
| | GTGGGTCCGCCTCGTGCTGGCAGCC*GGGAGGACACC |
| | CCAGAGCAAGCGGCCCAGGTGGGCGGGGAGGGTCTCG |
| | TCACAGGGGCAGCTGACAGATAGAGGCCCCCGCGAGG |
| | CAGATGCTTGATCCTGGCAgTTATACTGGGTTC**GC |
| | ACAACTTTGCCTGAACAAGGGGCCCTCCGAACAGACA |
| | CAGACGCAACCCAGTCGAGCcaggCTCAGCACAgAAA |
| | ATGCACTGACACGCAAAACCCTCATCTggggGCCTGG |
| | CGGGcAtCCCGCCCCAGGAGCCAAGGCCCGTGCCCCC |
| | TGGCAGCGCTGGACACGGTCCTCTGTGGGCGGTGGGG |
| | TCgGGGCTGTGGTGACGGTGGCATCGGGGAGCCTGTG |
| | CCCCCTCGCTGAAAGGGGGGAGAGGCTCAAGAGGGGA |
| | CAGAAATGTCCTCCCCTAGGAAGAGCTCGGACGGGGG |
| | CGGGGGGGTGGTGTCCGACAGACAGATGCCCGGGACC |
| | GACAGACCTGCCGAGGGAAGAGGGCACCTCGGTCGGG |
| | TTAGGCTCCAGGCAGCACGAGGGAGCGAGGCTGGGAG |
| | GGTGAGGACATGGGAGCGTGAGGAGGAGCTGGAGACT |
| | TCAGGAGGCCCCCAGGTCCGGGCTTCGGGCTCTGAGA |
| | TGCTCGGAGGGAAGGTGAGTGAGGCCAGCTGTGGCTG |
| | ACCTGACCTCAgGGgGACAAGGCTCAGCCTGGGACTC |
| | TGTGTCCCCATCGCCTGcACAGGGGATTCCCCTGATG |
| | GACACTGAGCCAACGAGCTCCCGTCTCTGCCCGACCC |
| | CCAGGTCAGCCGAAgGCCaCTCCCAGGGTCAACCTCT |
| | TCCGGCCCTCCTCTGAGGAGCTCGGCACCAACAAGGC |
| | CACCCTGGTGTGTCTAATAAGTGACTTCTACCCGGGC |
| | GCCGTGACGGTGACGTGGAAGGCAGGGGGCACCACCG |
| | TCACGCAGGGCGTGGAGACCACCAAGCCCTCGAAACA |
| | GAGCAACAACAAGTACGCGGCCAGGAGCTACCTGGCC |
| | CTGTCCGCCAGTGACTGGAAATCTTCCAGCGGCTTCA |
| | CCTGCCAGGTCACCCACGAGGGGACCATTGTGGAGAA |
| | GACAGTGACGCCCTCCGAGTGCGGCTAGGTCCCTGGG |
| | CCCCCACCGTCAGGGGGCTGGAGCCACAGGACCCCCG |
| | CGAGGGTcTCCCCGCGACCGTGGTCCAGCCCAGCCGT |
| | TCCTCCTGCACCTGTCAACTCCCAATAAACCGTCCTC |
| | CTTGTCATTCAGAAATCATGCTCTCCGCTCACTTGTG |
| | TCTACCCATTTTCGGGCTTGCATGGGGTCATCCTCGA |
| | AGGTGGAGAGAGTCCCCCTTGGCGTTGGGgAAATCGA |
| | GGGGGGCGGGGGGAGGGCTGAGGCATGTGCCAGCGAG |
| | GGGGGTCAGCTGCACGCCCCTGAGGACCTTCTAGAAC |
| | CAGGGGCGTGGGGCCAGCGCCAGAGTGGAAGGCTGTC |
| | CACTTTTCCCCCGGGCCCCCAGGCTCCCTCCTCCGTG |
| | TGGACCTTGTCCACCTCTGACTGGCCCAGCCACTCAT |
| | GCATTGTTTCCCCGAAACCCCAGGAGGATAGCTGAGC |
| | ACGCGACAGTGTCGCGCTGTGAGGGCCTGTGTCCATT |
| | TCAGGACGACCCGCATGTACAGCGTGACCACTCTGGT |
| | GACGCCCACTCACCACGTCCTAGAGCCCCACGCCCAG |
| | CCCGATCCTTAGGGGCACAGCCAGCTCCGACGGCCCG |
| | GGGGACACCACCCTCTGCCGGTTGCCCAGGCCGTCCC |
| | TGTCAGACGCACCACAGGGCCCTCCGTCGGGAGACGC |
| | TGCTCCGTGATCGCTCGGTCCCCTCAGGTAGCCTTCC |
| | ACCCGCGTGTGTCCCGAGGTCCCAGATGCAGCAAGGC |
| | CGCTGGGACAACGCCAGATGTCTGCTGTCCGCGACGG |
| | TCAGAAGCCAGCCCACGCCTGGCCCACCACCACTGGC |
| | TAACGTCCAAGTGTCCATAGGCTCGGGAcCTCcAaGT |
| | CCAGGTTCTGCGTCTGGGATTGCGCCATGGGTCTGCG |
| | TGGAATGATGCACTTGGAGgAgGTCAGcATGGGATGc |
| | GGAACTTGTCTAGcGCTCCTCAGATCCAcAGcTGCCT |
| | GtGAgAcacacacacacacacacacacaccAAAcaCG |
| | cATGCACGCACGCGGGCACACACGGTATTACAGAGAT |
| | GGCGACGGTAGCTGTGCCTCGAGGCCGAGTGGAGTGT |
| | CTAGAACTCTCGGGGGTCCCCTCTGCAGACGACAGTG |
| | CTCCATCCCCCCCGTGCCCTGAAGGGCTCCTCACTCT |
| | CCCATCAGGATCTCTCCAAGCTGCTGACCTGGAGAGG |
| | AAGGGGCCTGGGACAGGCGGGGACACTCAGAGGTGCG |
| | TGGTGGCGCTGGTCTGCCTGGGCTTGGAGGGCTCCGG |
| | CCTTGCGACGGGTGAAGGTGCAGGTGGGGAGAGGGCA |
| | CCCCCCTCACGCTCCCAGACCCAGAGCAGCCCCCGTG |
| | GGAGGGGCAGCCTGTGAGCCTCCAGCCAGATGCAGGT |
| | GGCCTGGGGTGGGGGGTGGAGGGGGCGGGAGGTTTAT |
| | GTTTGAGGCTGTATTCATCTGTGTAATATttTGGGGG |
| | GTGGGACCCATGTGAGGGTCCTCGGTGAGTGTCGCCT |
| | tttctttcctccttggggatccgagtgaaATcTGGGT |
| | GGATCTTCTCTCGGTTCTCGTCCGACTGGGGCTGAGG |
| | TCTGAACCTCGGTgGGGTCCGAAGAGGAGGCGCGTAG |
| | GCC*GGCTCcTCAGCCCCTCCAGCCCGACCCGCCGTG |
| | TTGACACAGGGTCCAGCTAAGGGCAGAGAT***GGCT |
| | GCTAGTCGAGGGCCAGGCTcTGAGAGCCAAGGGCGGT |
| | GCCCAAGGAAGCGTTGCCGCAGGGACCCTGGGAGCAA |
| | AGGTCCTCACTCAGAGCCTGCAGCCCTGGgGTCTGAG |
| | GACAAGGAGGGAGTGAGGACTGGGCGTGGGGAGTTCA |
| | GGCgGGGACACGGGGTCCAGGGAGGTGAGAAAGGCGC |
| | TGGGAGGGGGCGGAGGGTGCGGGAGACTCCTCCTGGG |
| | GCGTGTGGGCTCGTGGTCCTTGTGAGGACCCTGAGGG |
| | *CTGAGGGGCGCCTGGGCGTAGGGACTTGGAGTGAGG |
| | GAGGCAGGGAGTGTCCCTTGAGAACGTGGCCTCCGCG |
| | GGCTGGGTCCCCCTCGTGCTCCCAGGAGGGAGGACAC |
| | GCGAGAGCAAGCGCCCCAGGTGGGCGGGGAGGGTGTG |
| | CTCACAGGGGCAGCTGACAGATAGAC*GgccCCCGCC |
| | AGACAGATGCTTGATCCTGGTCag***TACTGGGTTC |
| | GCcACTTCCCTGAACAGGGGCCCTCCGAACAGACACA |
| | GACGCAGACCaggCTCAGCACAgAAAATGCACTGACA |
| | CCCAAAACCCTGATCTGggGGGCTGGCCGGCATCCCG |
| | CCCCAGGACGCAAGGCCCCTGCCCCCTGGCAGCCCTG |
| | GACACGGTCCTCTGTGGGGGGTGGGGTCgGGGCTGTG |
| | GTGACGGTGGCATCGGGGAGCCTGTGCCCCCTCCCTG |
| | AAAGGGCGGAGAGGCTCAAGAGGGGACAGAAATGTCG |
| | TCCCCTAGGAAGACGTCGGAGGGGGGCGGGGGGGTGG |
| | TCTCCGACAGACAGATGGGCGGGACCGACAGACCTGC |
| | CGAGGGAAGAGGGCACCTCGGTCGGGTTAGGCTCCAG |
| | GCAGCACGAGGGAGCGAGGCTGGGAGGGTGAGGACAT |
| | GGGAGCCTGAGGAGGAGCTGGAGAGTTCAGCAGGCCC |
| | CCAGCTCCGGGCTTCGGGCTCTGAGATGCTCGGACGC |
| | AAGGTGAGTGACCCCACCTGTGGCTGACCTGACCTGA |
| | CCtCAGGGGGACAAGGCTCAGCCTGGGACTCTgTGTC |
| | CCCATCGCCTGCACAGGGGATTCCCCTGATGGACACT |
| | GAGCCAACGACCTGCCGTCTCTCCGCGACCCCCAGGT |
| | CAGCCCAAGGCCACTCCCACGGTCAACCTCTTCCCGC |
| | CCTCGTGTGAGGAGCTGGGCACCAACAAGGCCACCCT |
| | GGTGTGTGTA |
| Seq ID No.32 | |
| | GCCACGCCCACTCCATCATGCGGGGAGGGGATGGGCA |
| | GAGGCTCCAGAAAGAAGCTCCCTGGGGTGCAGGTTAA |
| | CAGCTTTCCCAGACACAGCCAGTACTAGAGTGAGGTG |
| | AATAAGACATCCTCGTTGCTTGTGAAATTTAGGAAGT |
| | GCCCCCAACATCAGTCATTAAGATAAATAATATTGAA |
| | TGCACTTTTTTTTTTTTTATTTTTTTTTTTTGCTTTT |
| | TAGGGCCTAATCTGCAGCatatggaagttcccaggct |
| | acaagtcgaaccagagctgcagctgccagcctacatc |
| | acagccacagcaacaccagatccgagccacatctgtg |
| | actaacactgcagttcacagcaacgccagatccttaa |
| | cccattgagtgaggccagggatcaaacccacatcctc |
| | atggatactagtctggttcgtaaaccactgagccaCA |
| | AGGGGAACTCCTGAATGCAATATTTTTGAAAATTGAA |
| | ATTAAATCTGTCACTCTTTCACTTAAGAGTCCCCTTA |
| | GATTGGGGAAAATTTAAATATCTGTCATCTTAGTGCA |
| | TCTTTGCTCATATGATGTGAATAAAATCCCAAAATCC |
| | ATATGAATGAAGCATCAAAATGTACATGAAGTCAGGC |
| | TGACCCTGCACTGCGGTCACTTGCCTCATGTACCCCC |
| | CAGCTCAAAGGAAGATGCAGAAAGGAGTCCAGCCCCT |
| | ACACCGCCACCTGCCCCCACCACTGGAGCCCCTCAGG |
| | TCTCCCACCTCCTTTTCTGAGCTTCAGTCTTCCTGTG |
| | GCATTGCCTACCTCTACAGCTGCCCCCTACTAGGCCC |
| | TCCCCCTGGGGCTGAGCTCCAGGCACTGGACTGGGAA |
| | AGTTAGAGGTTAAAGCATGGAAAATTCCCAAAGCCAC |
| | CAGTTCCAGGCTGCCCCCCACCCCACCGCCACGTCCA |
| | AAAAGGGGCATCTTCCCAGATCTCTGGCTGGTATTGG |
| | TAGGACCCAGGACATAGTCTTTATACCAATTCTGCTG |
| | TGTGTCTTAGGAAAGAaactctccctctctgtgcttc |
| | agtttcctcatcaataaaAGGAGCAGGCCAGGTTGGA |
| | GGGTCTGTGACGTCTGCTGAAGCAGCAGGATTCTCTC |
| | TCCTTTTGCTGGAGGAGAACTGATCCTTCACCCCCAG |
| | GATCAACAGAGAAGCCAAGGTCTTCAGCCTTCCTGGG |
| | GACCCCTCAGAGGGAACTCAGGGCCACAGAGCCAGAC |
| | CCTGATGCCAGAACCTTTGTCATATGCCCAGAGGGAG |
| | ACTTCATCCCGCTCCTGGTCAGACCCTCCAGGCCCCA |
| | ACAGTGAGATGCTGAAGATATTAAGAGAAGGGCAAGT |
| | CAGcTTAAGTTTGGGGGTAGACGGGAACAGGGAGTGA |
| | GGAGATCTGGCCTGAGAGATAGGAGCCCTGGTGGCCA |
| | CAGGAGGACTCTTTGGGTCCTGTCGGATGGACACAGG |
| | GCGGCCCGGGGGCATGTTGGAGCCCGGCTGGTTCTTA |
| | CCAGAGGCAGGGGGCACCCTCTGACACGGGAGCAGGG |
| | CATGTTCCATACATGACACACCCCTCTGCTCCAGGGC |
| | AGGTGGGTGGCGGCACAGAGGAGCCAGGGACTCTGAG |
| | CAAGGGGTCCACCAGTGGGGCAGTTGGATCCAGACTT |
| | CTCTGGGCCAGCGAGAGTGTAGCCCTCAGCCGTTCTC |
| | TGTCCAGGAGGGGGGTGGGGCAGGCGTGGGCGGCGAG |
| | AGCTCATCCCTCAAGGGTTCCCAGGGTGCTGGGAGAC |
| | CCAGATTTCCGACCGCAGCCACCACAAGAGGATGTGG |
| | TCTGCTGTGGCAGCTGCCAAGACCTTGCAGCAGGTGC |
| | AGGGTGGGGGGGTGGGGGCACCTGGGGGCAGCTGGGG |
| | TCACTGAGTTCAGGGAAAACCCCTTTTTTCCCCTAAA |
| | CCTGGGGCCATCCCTAGGGGAAACCACAACTTCTGAG |
| | CCCTGGGGAGTGGCTGGTGGGAGGGAAGAGCTTCATC |
| | CTGGACCCTGGGGGGGAACCCAGCTCCAAAGGTGCAA |
| | GGGGCCGAGGTGCAAGGCTAGAGTGGGCCAAGCACCG |
| | GAATGGCCAGGGAGTGGGGGAGGTGGAGCTGGACTGG |
| | ATCAGGGCCTCCTTGGGACTCCCTACACCCTGTGTGA |
| | CATGTTAGGGTACCCACACCCCATCACCAGTCAGGGC |
| | CTGGCCCATCTCCAGGGCCAGGGATGTGCATGTAAGT |
| | GTGTGTGAGTGTGTGTGTGTGGTGTAGTACACCCCTT |
| | GGCATCCGGTTCCGAGGCCTTGGGTTCCTCCAAAGTT |
| | GCTCTCTGAATTAGGTCAAACTGTGAGGTCCTGATCG |
| | CGATCATCAACTTCGTTCTCCCCACCTCCCATCATTA |
| | TCAAGAGCTGGGGAGGGTCTGGGATTTCTTCCCACCC |
| | ACAAGCCAAAAGATAAGCCTGCTGGTGATGGCAGAAG |
| | ACACAGGATCCTGGGTCAGAGACAAAGGCGAGTGTGT |
| | CACAGCGAGAGAGGCAGCCGGACTATCAGCTGTCACA |
| | GAGAGGCGTTAGTCCGCTGAACTCAGGCCCCAGTGAC |
| | TCCTGTTCCACTGGGCACTGGCCCCCCTCCACAGCGC |
| | CCCCAGGCCCCAGGGAGAGGCGTCACAGGTTAGAGAT |
| | GGCCCTGCTGAACAGGGAACAAGAACAGGTGTGCGGC |
| | ATCCAGCGCCCCAGGGGTGGGACAGGTGGGCTGGATT |
| | TGGTGTGAAGCCCTTGAGCCCTGgAACCCAAcCACAG |
| | CAgGGCAGTTGGTAGATGCCATTTGGGGAGAGGCCCC |
| | AGGAGTAAGGGCCATGGGCCCTTGAGGGGGCCAGGAG |
| | CTGAGGACAGGGAGAGAGAGGGCGCAGGGAGAGGACA |
| | GGGCCATGAGGGGTGCAGTGAGATGGCCACTGCCAGC |
| | AGGGGCAGCTGCCAACCCGTCCAGGGAACTTATTCAG |
| | GAGTCAGGTGGAGGTGCCATTGACCCTGAGGGCAGAT |
| | GAAGCCCAGGGCAGGCTAGGTGGGGTGTGAAGACCGG |
| | AGGGGACAGAGCTCTGTCCCTGGGCAGCACTGGCCTC |
| | TCATTCTGCAGGGCTTGACGGGATCCCAAGGCGTGCT |
| | GCCCCTGATGGTAGTGGCAGTACCGCCCAGAGCAGGA |
| | CCCCAGCATGGAAACCCCAACGGGACGCAGCCTGCGG |
| | AGCCCACAAAACGAGTAAGGAGCCGAAGCAGTGATGG |
| | CACGGGGAGTGTGGACTTCCGTTTGATGGGGCCCAGG |
| | CATGAAGGACAGAATGGGACAGCGGCCATGAGCAGAA |
| | AATCAGCCGGAGGGGATGGGCCTAGGCAGACGCTGGC |
| | TTTATTTGAAGTGTTGGCATTTTGTCTGGTGTGTATT |
| | GTTGGTATTGATTTTATTTTAGTATGTCAGTGACATA |
| | CTGACATATTATGTAACGACATATTATTATGTGTTTT |
| | AAGAAGCACTCCAAGGGAACAGGCTGTCTGTAATGTG |
| | TCCAGAGAAGAGAGCAAGAGCTTGGCTCAGTCTCCCC |
| | CAAGGAGGTCAGTTGGTCAACAGGGGTCCTAAATGTT |
| | TCCTGGAGCCAGGCGTGAATCAAGGGGgTCATATCTA |
| | CACGTGGGGGAGAGCCATGGACCATTTTCGGAGCAAT |
| | AAGATGGCAGGGAGGATACCAAGCTGGTcTTACAGAT |
| | CCAGGGCTTTGACCTGTGACGCGGGCGCTCCTGCAGG |
| | CAAAGGGAGAAGCCAGCAGGAAGCTTTCAGAACTGGG |
| | GAGAACAGGGTGCAGACCTCCAGGGTCTTGTAGAACG |
| | CACCCTTTATCCTGGGGTCCAGGAGGGGTCACTGAGG |
| | GATTTAAGTGGGGGAGCATCAGAACCAGGTTTGTGTT |
| | TTGGAAAAATGGCTCCAAAGCAGAGACCAGTGTGAGG |
| | CCAGATTAGATGATGAAGAAGAGGCAGTGGAAAGTCG |
| | ATGGGTGGCCAGGTAGCAAGAGGGCCTATGGAGTTGG |
| | CAAGTGAATTTAAAGTGGTGGCACCAGAGGGCAGATG |
| | GGGAGGAGCAGGCACTGTCATGGACTGTCTATAGAAA |
| | TCTAAAATGTATACGCTTTTTAGCAATATGCAGTGAG |
| | TCATAAAAGAACACATATATATTTAAATTGTGTAATT |
| | GGACTTCTAAGGATTCATGCCAAGGGGGGAAAATAAT |
| | CAAAGATGTAAGCAAAGGTTTACAAACAAGAACTCAT |
| | CATTAATGTTGGTTGTTGTTATTTCAACGATATTATT |
| | ATTATTACTATTATTATTATTATTTATTttgtctttt |
| | tgcatttctagggccactcccacggcatagagaggtt |
| | cccaggctaggggtcaaatcggagctacagctgccgg |
| | cctacgccagagccacagcaacgcaggatctgagcca |
| | cagcaatgcaggatctacaccacagctcatggtaacg |
| | ctggatccttaacccaatgagtgaggccagggatcga |
| | acctgtaacttcatggttcctagtcggattcattaac |
| | cactgagccacgacaggaactccAACATTATTAATGA |
| | TGGGAGAAAACTGGAAGTAACCTAAATATCCAGCAGA |
| | AAGGGTGTGGCCAAATACAGCATGGAGTAGCCATCAT |
| | AAGGAATCTTACACAAGCCTCCAAAATTGTGTTTCTG |
| | AAATTGGGTTTAAAGTACGTTTGCATTTTAAAAAGCC |
| | TGCCAGAAAATACAGAAAAATGTCTGTGATATGTCTC |
| | TGGCTGATAGGATTTTGCTTAGTTTTAATTTTGGCTT |
| | TATAATTTTCTATAGTTATGAAAATGTTCACAAGAAG |
| | ATATATTTCATTTTAGCTTCTAAAATAATTATAACAC |
| | AGAAGTAATTTGTGGTTTAAAAAAATATTCAACACAG |
| | AAGTATATAAAGTAAAAATTGaggagttcccatcgtg |
| | gctcagtgattaacaaacccaactagtatccatgagg |
| | atatggatttgatccctggccttgctcagtgggttga |
| | ggatccagtgttgctgtgagctgtggtgtaggttgca |
| | gacacagcactctggcgttgctgtgactctggcgtag |
| | gccggcagctacagctccatttggacccttagcctgg |
| | gaacctccatatgcctgagatacggcccTAAAAAGTC |
| | AAAAGCCAAAAAAATAGTAAAAATTGAGTGTTTCTAC |
| | TTACCACCCCTGCCCACATCTTATGCTAAAACCCGTT |
| | CTCCAGAGACAAACATCGTCAGGTGGGTCTATATATT |
| | TCCAGCCCTCCTCCTGTGTGTGTATGTCCGTAAAACA |
| | GACACACACACACACACACGCACACACACACACACGT |
| | ATGTAATTAGCATTGGTATTAGTTTTTCAAAAGGGAG |
| | GTCATGCTCTACCTTTTAGGCGGCAAATAGATTATTT |
| | AAACAAATCTGTTGACATTTTCTATATCAACCCATAA |
| | GATCTCCCATGTTCTTGGAAAGGCTTTGTAAGACATC |
| | AACATCTGGGTAAACCAGCATGGTTTTTAGGGGGTTG |
| | TGTGGATTTTTTTCATATTTTTTAGGGGACACCTGCA |
| | gcatatggaggttcccaggctaggggttgaatcagag |
| | ctgtagctgccggcctacaccacagccacagcaacgc |
| | cagatccttaacccactgagaaaggccagggattgaa |
| | cctgcatcctcatggATGCTGGTCAGATTTATTTGTG |
| | CTGAGCCAGAACAGGAACTCCCTGAACCAGAATGCTT |
| | TTAACCATTCCACTTTGCATGGACATTTAGATTGTTT |
| | CCATTTAAAAATACAAATTACAaggagttcccgtcgt |
| | ggctcagtggtaacgaattggactaggaaccatgagg |
| | tttcgggttcgatccctggccttgctcggtgggttaa |
| | ggatccagcattgatgtgagatatggtgtaggtcgca |
| | gacgtggctcggatcccacgttgctgtggctctggcg |
| | taggccggcaacaacagctccgattcgacccctagcc |
| | TGggaacctccatgtgccacaggagcagccctaGAAA |
| | AGGCAAAAAGACAAAAAAATAAAAAATTAAAATGAAA |
| | AAATAAAATAAAAATACAAATTAGAAGAGACGGCTAG |
| | AAGGAAATCCCCAAGTGTGTGCAAATGCCATATATGT |
| | ATAAAATGTACTAGTGTCTCCTCGCGGGAAAGTTGCG |
| | TAAAAGTGGGTTGGCTGGACAGAGAGGACAGGCTTTG |
| | ACATTCTCATAGGTAGTAGCAATGGGGTTCTCAAAAT |
| | GCTGTTCCAGTTTACACTCAGCATAGCAAATGACAGT |
| | GCGTCTTCCTCTCCACCCTTGCCAATAATGTGAGAGG |
| | TGGATCTTTTTCTATTTTGTGTATCTGAGAAGCAAAA |
| | AATGAGAAGAggagttcctgtcgtggtgcagtggaga |
| | caaatctgactaggaaccatgaaatttcgggttcaat |
| | ccctggcctcactcagtaggtaaaggatccagggttg |
| | cagtgagctgtggggtaggtcgcagacacagtgcaaa |
| | tttggccctgttgtggctgtggtgtaggccggcagct |
| | atagctccaattggacccctagcctgggaacctcctt |
| | atgccgtgggtgaggccctAAAAAAAAGAGTGCAAAA |
| | AAAAAAAATAAGAACAAAAATGATCATCGTTTAATTC |
| | TTTATTTGATCATTGGTGAAACTTATTTTCCTTTTAT |
| | ATTTTTATTGACTGATTTTATTTCTCCTATGAATTTA |
| | CCGGTCATAGTTTTGCCTGGGTGTTTTTACTCCGGTT |
| | TTAGTTTTGGTTGGTTGTATTTTCTTAGAGAGCTATA |
| | GAAACTCTTCATCTATTTGGAATAGTAATTCCTCATT |
| | AAGTATTTGTGCTGCAAAAAATTTTCCCTGATCTGTT |
| | TTATGCTTTTGTTTGTGGGGTCTTTCACGAGAAAGCC |
| | TTTTTAGTTTTTACAGCTCAGCTTGGTTGTTTTTCTT |
| | GATTGTGTCTGTAATCTGCGGCCAACATAGGAAACAC |
| | ATTTTTACTTTAGTGTTTTTTTCCTATTTTCTTCAAG |
| | TACGTCCATTGTTTTGGTGTCTGATTTTACTTTGCGT |
| | GGGGTTTGTTTTTGTGTGGCAGGAATATAAACTTATG |
| | TATTTTCCAAATGGAGAGCCAATGGTTGTATATTTGT |
| | TGAATTCAAATGCAACTTTATCAAACACCAAATCATC |
| | GATTTATCACAACTCTTCTCTGGTTTATTGATCTAAT |
| | GATCAATTCCTGTTCCACGCTGTTTTAATTATTTTAG |
| | CTTTGTGGATTTTGGTGCCTGGTAGAGAACAAAGCCT |
| | CCATTATTTTCATTCAAAATAGTCCCGTCTATTATCT |
| | GCCATTGTTGTAGTATTAGACTTTAAAATCAATTTAC |
| | TGATTTTCAAAAGTTATTCCTTTGGTGATGTGGAATA |
| | CTTTATACTTCATAAGGTACATGGATTCATTTGTGGG |
| | GAATTGATGTCTTTGCTATTGTGGCCATTTGTCAAGT |
| | TGTGTAATATTTTACCCATGCCAACTTTGCATATTGT |
| | ATGTGAGTTTATTCCCAGGGTTTTTAATAGGATGTTT |
| | ATTGAAGTTGTCAGTGTTTCCACAATTTCATCGCCTC |
| | AGTGCTTACTGTTTGCATAAAAGGAAACCTACTCACT |
| | TTTGCCTATTGCTCTTGTATTCAATCATTTTAGTTAA |
| | CTCTTGTGTTAATTTTGAGAGTTTTTCAGCTGACTGT |
| | CTGGGGTTTTCTTTAATAGACTAGCCCTTTGTCTGTA |
| | AAGAATAATTTTATCGAATTTTTCTTAACACTGAGAC |
| | TCTCCCCACCCCCACCCCCGCTCATGTCGTTTCATTG |
| | GGTCAAATCTGTAGAATACAATAAAAGTAAGAGTGGG |
| | AACCTTAGCCTTTAAGTCGATTTTGCCTTTAAATGTG |
| | AATGTTGCTATGTTTCGGGACATTCTCTTTATCAAGT |
| | TGCGGATGTTTCCTTAGATTATTAACTTAATAAAAGA |
| | CTGGATGTTTGCTTTCTTCAAATCAGAATTGTGTTGA |
| | ATTTATATTGCTATTCTGTTTAATTTTGTTTCAAAAA |
| | ATTTACATGCACACCTTAAAGATAACCATGACCAAAT |
| | AGTCCTCCTGCTGAGAGAAAATGTTGGCGCCAATGCC |
| | ACAGGTTACCTCCCGACTGAGATAAACTACAATGGGA |
| | GATAAAATCAGATTTGGCAAAGCCTGTGGATTCTTGC |
| | CATAACTCTCAGAGCATGACTTGGGTGTTTTTTCCTT |
| | TTCTAAGTATTTTAATGGTATTTTTGTGTTACAATAG |
| | GAAATCTAGGACACAGAGAGTGATTCAATGAGGGGAA |
| | CGCATTCTGGGATGACTCTAGGCCTCTGGTTTGGGGA |
| | GAGCTCTATTGAAGTAAAGACAATGAGAGGAAGCAAG |
| | TTTGCAGGGAACTGTGAGGAATTTAGATGGGGAATGT |
| | TGGGTTTGAGGTTTCTATAGGGCAGGCAAGCAGAGAT |
| | GCACTCAGGAGGAAGAAGGAGCATAAATCTAGAGGGA |
| | AAAAGAGAGGTCAGGACTGGAAATAGAGATGCGAGAC |
| | ACCAGGGTGGCAGTCAGAGAGCACAGTGTGGGTGAGA |
| | AGACAGTGGAAGAACACAAGGGACAGAGAGGGATCTC |
| | CAACTTCACTGGGATGAGGGCCTTGTTGGCCTTGACC |
| | TGAGAGATTTCCAGGAGTTGAGGGTGGGAAGGAGAGG |
| | GCTCCTGCACATGTCCTGACATGAAAGGGTGCCCAGC |
| | ATATGGGTGCTTGGAAGACATTGTTGGACAGATGGAT |
| | GGATGATGGATGATGGATGAATGGATGGATGGAAGAT |
| | GATGGATAAATGGATGATGGATGGATGGACAGAAGGA |
| | CAAAGAGATGGACAGAAAGAGAGTGATCTGAGAGAGC |
| | AGAGAAGGCTTCATGAAAGGACAGGAACTGAACTGTC |
| | TCAGTGGGTGGAGACAATGGTGTAGGGGGTTTCCACA |
| | TGGAGGCACCAGGGGTCAGGAATAATCTAGTGTGCAC |
| | AGGCCGAGGAAGGAAGCTGTCTGCAGGAAATTGTGGG |
| | GAAGAACCTGAGAGTCCTTAAATGAGGTCAGGAGTGG |
| | TCAGGAGGGTGTGATCAGGTAAGGACTCATGTCCATC |
| | ATCACATGGTCACCTAAGGGCATGTAGCTGTCAGCAT |
| | CTCCATCAGGACAGTCTCAGAATGGGGGCGGGGTCAC |
| | ACACTGGGTGACTCAAGGCGTGGGTCATGGCTGCCTC |
| | GGAGGTGGGCCTGGGGATGGGGACACCTCGAGACCAT |
| | GGGCCGGCCCAGGGCTGGACTGGcctctggtgggcta |
| | gctacccgtccaagcaacacaggacacagccctacct |
| | gctgcaaccctgtgcccgaaacgcccatctggttcct |
| | gctccagcccggccccagggaacaggactcaggtgct |
| | agcccaatggggttttgttcgagcctcagtcagcgtg |
| | gTATTTGTCCGGCAGCGAGACTCAGTTCACCGCCTTA |
| | ttaagtggttctcatgaatttcctagcagtcctgcac |
| | tctgctatgccgggaaagtcacttttgtcgctggggg |
| | ctgtttccccgtgcccttggagaatcaaggattgccc |
| | aactttctctgtgggggaggtggctggtcttggggtg |
| | accagcaggaagggccccaaaagcaggagcagctgcc |
| | tccagAATACAACTGTCGGGTAGAGCTCAAACAGGAG |
| | GCCTGGACTGGGGTTTAACCACCAGGGCGGCACGAAG |
| | GAGGGAGGCTGGGAGGGTGAGGACATGGGAGCGTGAG |
| | GAGGAGCTGGAGACTTCAGGAGGCGCCGAGCTCCGGG |
| | GTTGGGGCTGTGAGATGCTGGGACGCAAGGTGAGTGA |
| | CGCGACCTGTGGCTGACGTGACCTCAGGGGGACAAGG |
| | GTCAGCCTGAGACTGTGTGTCCCCATCGCCTGGACAG |
| | gggattcccctgatggacactgagccaacgacctccc |
| | gtctctccccgacccccaggtcagcccaaggccgccc |
| | ccacggtcaacctcttcccgccctcctctgaggagct |
| | cggcaccaacaaggccaccctggtgtgtctaataagt |
| | gacttctacccgAAGGGcGAATTCCAGCACACTGGCG |
| | GCCGTTACTAGTGGATCGGAGCTGGGTACCAAGCTTG |
| | ATGCATAGCTTGAGTATCTA |
| Seq ID No.33 | agatctttaaaccaccgagcaaggccagggatcgaac |
| | ccgcatcctcatgaatccagttgggttcgttaaccgc |
| | tgaaccacaatgggaactcctGTCTTTCACATTTAAT |
| | TCACAACCTCTCCAGGATTGTGGGGGTGGGTGGGGAA |
| | TCCTAGGTACCCACTGGGAAAGTAATCCAAGGGGAGA |
| | GGCTCACGGACTcTAGGGATCGGCGGAGGAGGGAAGG |
| | TATCTCCCAGGAAACTGGCCAGGACACATTGGTCCTC |
| | CGCGCTCCCCTTCCTCCCACTCCTCCTCCAGACAGGA |
| | GTGTGCCCACCCCCTGCCACCTLTCTGGCCAGAACTG |
| | TCCATGGCAGGTGACCTTCACATGAGCCCTTCCTCCC |
| | TGCCTGCCCTAGTGGGAGCCTCGATACGTCCCCCTGG |
| | ACCCCGTTGTCCTTTCTTTCCAGTGTGGCCGTGAGCA |
| | TAACTGATGCCATCATGGGCTGCTGAGCCACCCGGGA |
| | CTGTGTTGTGCAGTGAGTCACTTCTCTGTCATCAGGG |
| | CTTTGTAATTGATAGATAGTGTTTCATCATCATTAGG |
| | ACCGGGTGGCCTGTATGCTCTGTTAGTCTCCAAACAG |
| | TGATGAAAACCTTCGTTGGCATAGTCCCAGCTTCCTG |
| | TTGCCCATCCATAAATCTTGACTTAGGGATGGAGATC |
| | CTGTCTCCAAGCAACCACCCGTCCCGTAGGCTAACTA |
| | TAAAACTGTCCCAATGGCCCTTGTGTGGTGCAGAGTT |
| | CATGCTTCCAGATCATTTCTCTGCTAGATCCATATCT |
| | CACCTTGTAAGTCATCCTATAATAAACTGATCCATTG |
| | ATTATTTGCTTCTGTTTTTTCCATCTCAAAACAGCTT |
| | CTCAGTTCAGTTCGAATTTTTTATTCCCTCCATCCAC |
| | CCATACTTTCCTCAGCCTGGGGAACGCTTGCGCCCAG |
| | TCCCATGCCCTTCCTCGCTCTCTGCCCAGCTCAGCAG |
| | CTGCCCACCGTCACCCTTCCTGTCACTCCCTAGGACT |
| | GGACCATCCACTGGGGCCAGGACACTCCAGCAGCCTT |
| | GGCTTCATGGGCTCTGAAATCCATGGCCCATCTCTAT |
| | TCCTGACTGGATGGCAGGTTCAGAGATGTGAAAGGTC |
| | TAGGAGGAAGCCAGGAAGGAAACTGTTGCATGAAAGG |
| | CCGGCCTGATGGTTCAGTACTTAAATAATATGAGCTC |
| | TGAGCTCCCCAGGAACCAAAGCATGGAGGGAGTATGT |
| | GGCTGAGAATCTCTCTGAGATTCAGCAAAGCCTTTGC |
| | TAGAGGGAAAATAGTGGCTCAACCTTGAGGGCCAGCA |
| | TCTTGCACCACAGTTAAAAGTGGGTATTTGTTTTACC |
| | TGAGGCCTCAGCATTATGGGAACCGGGCTCTGACACA |
| | AACACAGGTGCAGCGCGGCAGCCTCAGAACACAGGAA |
| | CGACCACAAGCTGGGACAGCTGCCCCTGAACGGGGAG |
| | TGCACCATGCTTCTGTCTCGGGTACCACGAGGTCACC |
| | ATCCCTGGGGGAGGTAGTTCCATAGCAGTAGTCCCCT |
| | GATTTCGCGCCTCGGGCGTGTAGCCAGGCAAGCTCGT |
| | GCCTCTGGACCCAGGGTGGACCCTTGCTCCCCACTAC |
| | CGTGCACATGCCAGACAGTCAAGACCACTCCCACCTC |
| | TGTCTGAGGGGCGCTTGGGTGTCCCAGGGCGCCCGAG |
| | CTGTCCTCTACTGATGGTTCTTCCACGTGGGTACAAA |
| | AGAGGCGAGGGACACTTTCTCAGGTTTTGCGGCTCAG |
| | AAAGGTACCTTCCTAGGGTTTGTCCACTGGGAGTCAC |
| | CTCCGTTGCATCTCAATGTGAGTGGGGAAAACTGGGT |
| | CCCATGGGGGGATTAGTGCCACTGTGAGGCCCCTGAA |
| | GTCTGGGGCCTCTAGACACTATGATGATGAGGGATGT |
| | GGTGAAAAACGCCACCCCAGCCCTTCTTGCCGGGACC |
| | CTGGGCTGTGGCTCCCCCATTGCACTTGGGGTCAGAG |
| | GGGTGGATGGTGGCTATGGTGAGGCATGTTTCGCATG |
| | AGCTGGGGGCACCCTGGGTGACTTTCTCCTGTGAATC |
| | CTGAATTAGCAGCTATAACAAATTGCCCAAACTCTTA |
| | GGCTTAAAACAACAGACATTTATTGCTCTGGGTCCCA |
| | GGGTCAGAAGTCCAAAATGAGTCCTATAGGCTAAATT |
| | TGAGGTGTCTCTGGGTTGAGCTCCTCCTGGAAGCCTT |
| | TTCCAGCCTCTAGAGTCGCAAGTCCTTGGCTCTGGGC |
| | CCCTCCCTCAAGCTTCAAAGCCACAGAAGCTTCTAAT |
| | CTGTCTCCCTTCCCCTCTGACGTCTGCTCGCATCCTC |
| | ATACCCTGTCCCCTCACTCTGACCCTCCTGCCTCCCT |
| | CTTTCCCTTATAAAGACCCTGCATGGGGCCACGGAGA |
| | TAATCGAGGGTAATCGCCCCTCTTCGAGCCCTTAACT |
| | CCATCCCATCTGCAAAATCCCTGTCACCCCATAATGG |
| | ACCTACTGATGGTCTGGGGGTTAGGACGTGGACAAGT |
| | TGGGGCCTTATTCATGTGATCACAACTCCAGTTCCCA |
| | GACCCGCAGACCCCCGGGCATTAGGGAAAGTTCTCCC |
| | AGTTCCTCTCCGTCTGTGTCCTGCCCAGTCTCCAGGA |
| | TGGGCCACTCCCGAGGGCCCTTCAGCTCAGGCTCCCC |
| | CTCCTTTCTCCCTGGCCTCTTGTGGCCCCATCTCCTC |
| | CTCCGCTCACAGGGAGAGAACTTTGATTTCAGCTTTG |
| | GCTCTGGGGCTTTGCTTCCTTCTGGCCATTGGCTGAA |
| | GGGCGGGTTTCTCCAGGTCTTACCTGTCAGTCATCAA |
| | ACCGCCCTTGGAGGAAGACCCTAATATGATGCTTACC |
| | CTACAGATGGAGACTCGAGGCCCAGAGATCCTGAGTG |
| | ACCTGCTCAGATTCACAGCAGGGACTGAACCCGAGTC |
| | ACCTAGCGAACTCCAGGGCTCAGCGCTTTTTTTTTTT |
| | TTTTTCTTTTTgccttttcgagggccgctcccgcaac |
| | atatggagatttccaggctaggggtctaattggagca |
| | gtcgacactggcctaagccaaagccacagcaacaagg |
| | gcaagccgcttctgcagcctataccacagctcacggc |
| | aatgccggatccttaacccactgagcaaagccaggga |
| | ttgaacctgcaacctcatgtttcctagtcaaatttgt |
| | taaccactgacccatgacgggaactcccAGGGCTCAG |
| | CTCTTGACTCCAGGTTCGCAGCTGCCCTCAAAGCAAT |
| | GCAACCCTGGCTGGCCCCGCCTCATGCATCCGGCCTC |
| | CTCCCCAAAGAGCTCTGAGCCCACCTGGGCCTAGGTC |
| | CTCCTCCCTGGGACTCATGGCCTAAGGGTACAGAGTT |
| | ACTGGGGCTGATGAAGGGACCAATGGGGACAGGGGCC |
| | TCAAATCAAAGTGGCTGTCTCTCTCATGTCCCTTCCT |
| | CTCCTCAGGGTCCAAAATCAGGGTCAGGGCCCCAGGG |
| | CAGGGGCTGAGAGGGCGTCTTTCTGAAGGCCCTGTCT |
| | CAGTGCAGGTTATGGGGGTCTGGGGGAGGGTCAATGC |
| | AGGGCTCACCCTTCAGTGCCCCAAAGCCTAGAGAGTG |
| | AGTGCCTGCCAGTGGCTTCCCAGGCCCAATCCCTTGA |
| | CTGCCTGGGAATGCTCAAATGCAGGAACTGTCACAAC |
| | ACCTTCAGTCAGGGGCTGCTCTGGGAGGAAAAACACT |
| | CAGAATTGGGGGTTCAGGGAAGGCCCAGTGCCAAGCA |
| | TAGCAGGAGCTCAGGTGGCTGCAGATGGTGTGAACCC |
| | CAGGAGCAGGATGGGCGGCACTCCCCCCAGACCCTCC |
| | AGAGCCCCAGGTTGGGTGCCGTCTTCACTGGCGACAC |
| | CCGTGGGTGCACTTCTGCGCTTTCCCACGTAAAACCT |
| | TTAGGGCTCCCACTTTCTCCCAAATGTGAGACATCAC |
| | CAGGGCTCCCAGGGAGTGTCCAGAAGGGGATGTGGCT |
| | GAGAGGTCGTGACATCTGGGAGGCTCAGGCCCCACAA |
| | TGGACAGACGCCCTGCCAGGATGGTGCTGCAGGGCTG |
| | TTAGCTAGGCGGGGTGGAGATGGGGTACTTTGCCTCT |
| | CAGAGGCCCCGGCCCCACCATGAAACGTCAGTGACAC |
| | GCCATTTCCCTGAGTTCAGATACCTGTATCCTACTCC |
| | AGTCAGCTTCGCCACGAACCCCTGGGAGCGGAGGATG |
| | ATGCTGGGGCTGGAGCCACGACCAGCGCACGAGTGAT |
| | CCAGGTCTGCCAATCAGCAGTCATTTCCCAAGTGTTC |
| | CAGCCCTGCCAGGTCCCACTACAGCAGTAATGGAGGC |
| | CCCAGACACCAGTCGAGCAGTTAGAGGGCTGGACTAG |
| | CACCAGCTTTCAAGCCTCAGCATCTCAAGGTGAATGG |
| | CCAGTGCCCGTCCCCGTGGCCATCACAGGATCGGAGA |
| | TATGACCCTAGGGGAAGAAATATCCTGGGAGTAAGGA |
| | AGTGCCCATACTCAAGGATGGCCCCTCTGTGACGTAA |
| | CGTGTCCCTGAGGATTGTACTTGCAGGCGTTAAAACA |
| | GTAGAACGGCTGCCTGTGAACCCCCGCCAAGGGACTG |
| | CTTGGGGAGGCCGCCTAAACCAGAACACAGGCACTCC |
| | AGCAGGACGTGTGAACTCTGACCACCCTCAGCAAGTG |
| | GCACCCCGCGCAGCTTCCAAGGCAC |
| Seq ID No.34 | AACAAGATGCTACCCCACCAACAAAATTCACCGGAGA |
| | AGACAAGGACAGGGGGTTCCTGGGGTCCTGACAGGGT |
| | CACCAAAGAGGGTTCTGGGGCAGCAGCAACTCCAGCC |
| | GCCTCAGAACAGAGCCTGGAAGCTGTACCCTCAGAGC |
| | AGAGGCGGAGAGAGAAAGGGGCTCTTGGTGGGTCAGC |
| | AGGAGCAGAGGCTCAGAGGTGGGGGTTGCAGCCCCCC |
| | CTTCAACAGGCCAACACAGTGAAGCAGCTGACCCCTC |
| | CACGTTGGAGACCCCAGACTCCTGTCTCCCACGCCAC |
| | CTTGGTTTTTAAGGTAATTTTTATTTTATATCAGAGT |
| | ATGGTTGACTTACAATGTTGTGTTGGTTTCAGGTGTA |
| | CAGCAGAGTGATTCACTTCTACATAGACTCATATCTA |
| | TTCTTTCTCAGATTCTTTTCCCATATAGGTTATTACA |
| | GAATATTGAGTAGATCCCTGCTGATTACCCATTTTTA |
| | TAATTGTATATGTTAATCCCAAACTCCTAATTTATCC |
| | CTCCCCAGACTATGATTCTTTATATCTCTATCTGTTT |
| | CCTAATCTGTCTCCTCTAAGTCACCCTAGGAGAGCAG |
| | AGGGGTGACGTCTGTCCTGTCGTGGGCCAGCCACCTC |
| | TCTCCACCCAGGAATCCCTTGCATTTGGTGGCAAGGG |
| | CGGGGGCCCGCCCTAAAGAGAAAGGAGAACGGGATGT |
| | GGACAGGACACCGGGCAGAGAGGGACAAGCAGAGGAT |
| | GCCAGGGTAGGGAGGTCTGCAGGGTGGATGGTGGTCT |
| | GTCCGCAGGGAGGATGAGGCAGGAAGGGTGTGGATGT |
| | ACTCGGTGAGGCTGGCGCATGGCGTGGAGTGTCCTGA |
| | GCCCTGGGAGGCCTCAGCCCTGGATCAGATGTGTGAT |
| | TCCAAAGGGCCACTGCATCCAGAGACCGTTGAGTGGC |
| | CCATTGTCCTGAACCATTTATAGAACAGAGGACAAGC |
| | GGTACCTGACTAAGCTGGTCACAGATTGCATGAGGCT |
| | GATGCGAGGGTTGTCACGCCATCTCACAGGCAGGGAA |
| | AGTGATGCATATAGTGCAGAGCGAGGCAGAGGCCCTC |
| | CCAGTGCGCCGTGCCAGCCTGTGGCCGCCGTGCAGTG |
| | GCTGGACACTGAGGCCACACTGGGGCACCCTGTGGAG |
| | ATC |
| Seq ID No.35 | AGATCTGGCCAGGCCAGAGAAGCCCATGTGGTGACCT |
| | CCCTCCATCACTCCACGCCCTGACCTGCCAGGGAGCA |
| | GAAAGTAGGCCCAGGGTGGACCCGGTGGCCACCTGCC |
| | ACCCCATGGCTGGGAGAAGGGAGGGCCTGGGCAAAGG |
| | GCCTGGGAAGCCTGTGGTGGGACCCCAGACCCCAGGG |
| | TGGACAGGGAGGGTCCCACACCCACAGCCATTTGCTT |
| | CCCTCTGTGGGTTCAGTGTCCTCATCTCATCTGTGGG |
| | GAGGGGGCTGATAATGAATCTCCCCCATTGGGGTGGG |
| | CTTGGGGATTAAAGGGCCAGTGTGTGTGATATGCCTG |
| | GACCATAGTGACCCTCACCCTCCCCAGCCATTGCTGT |
| | CACCTTCCGGGCTCTTGCCCAGGCCTGCCTGACATGC |
| | TGTGTGACCCTGGGCAAGATGATCCCCCTTTCTGGGC |
| | CCCAGCCTTCCTCTCTGCTCCGGAAGTGCTTCCTGGG |
| | GAAACCTGTGGGCTGGATCCTATAGGAAACGTGTCCA |
| | ATTGCTGGATGCACAGAGGGGGAGGGAGGCCCTGGGC |
| | CTGGAGGGGCAGGGAGGCTCGAGGTGGGAGGAGGGTA |
| | GGGGCGAGTCCAGGGCAAGGAGGTGGGTGGGTAGGGT |
| | G |
| Seq ID No.36: | GATCTGTGTTCCATCTCAGAGCTATCTTAGCAGAGAG |
| | GTGCAGGGGCCTCCAGGGCCACCAAAGTCCAGGCTCA |
| | GCCAGAGGCAATGGGGTATCGATGAGCTACAGGACAC |
| | AGGCGTCAGCCCAGTGTCAGGGAGAATCACCTTGTTT |
| | GTTTTCTGAGTTCCTCTTAAAATAGAGTTAATTGGTC |
| | TTGGCCTTACGGTTTACAATAACAACTGCACCCTGTA |
| | AACAACGTGAAGAGTACAGAACAACAAATGGGGGAAA |
| | ACATATTTCACCTGAAAGAGCCACCGCTCATATTTTG |
| | ATGGATTTCCTTCTAGTTTAATCCTGTTTTAATTGTA |
| | AACTGTTAAAACAAACATAAATAAAGAAAATGCATCT |
| | GTAAAGTTTAAAAGTCATATCTATGGTGATGGTTGCA |
| | AAACACTGTGAATGTTCACTTTGAAATCGTGAACTCT |
| | ACGTGATATGCATGTCCCGTTAATTAACCTCACAGGC |
| | TCAGAATGTGGTTCATTATTTCTTTAATTTTCCTTTA |
| | ATTTTATGTCCTCTGTGTGTGCCCTTAAACCAACTAC |
| | TTTTCAGCTCTGCCTGTTTTTGACCTTCACATAGATG |
| | ACATTTGTGAGTGTTTTCTTTCTCAACACTGGGTCTG |
| | ATACCCACCCACGCTGTCTGCTGTCACTGCGGACGTG |
| | GAGGGCCACCACCCAGCTATGGCCCCAGCCAGGCCAA |
| | CACTGGATGAATCTGCCCCCAGAGCAGGGCCACCAAC |
| | ACTGGAGGTGCAGAGAGGGTTTCTTCAGGGCCATCAT |
| | TATCCAAGGCATTGTTTCTACTGTAAGCTTTCAAAAT |
| | GCTTCCCCTGATTATTAAAAGAAATAATAAGATGGGG |
| | GGAAAGTACAAGAAGGGAAGTTTCCAGCCCAGCCTGA |
| | AGATCGTGCTGGTTGTATCTGGAGCCTGTCTTCCTGA |
| | CAGGCCTCTATTCCCAGAGTTA |
| Seq ID No.37: | GGATCCTAGGGAAGGGAGGGCGGGGGCCTGGAGAAAG |
| | GGGGCCTAAAGGACATTCTCACCTATCCCACTGGACC |
| | cctgctgtgctctgagggagggagcagagagggggtc |
| | tgaggccttttcccagCTCCTCTGAGTCCCTCCTCCG |
| | AGCACCTGGACGGAAGCCCCTCCTCAGGGAGTCCTCA |
| | GACCCCTCCGCTCCAGCCAGGTTGGCCTGTGTGGAGT |
| | CCCCAGTAAGAATAGAATGCTCAGGGCTTCGAGCTGA |
| | GCCCTGGCTACTTGGGGGGGTGCTGGGGATTGGGGGT |
| | GCTGGGCGGGGAGCTGGGGTGTCACTAGATGCCAGTA |
| | GGCTGTGGGCTCGGGTCTGGGGGGTCTGCACATGTGC |
| | AGCTGTGGGAAGGCCCTATTGGTGGTACCCTCAGACA |
| | CATATGGGCCCTCAATTTGTGAGACCAGAGACGCCAG |
| | TCTGGCCTTCCCAGAACAGGTGGCGGTGGTGGGGGAG |
| | ATGTAGGGGGGCCTTCAGCCCAGGACCCCCAACGGCA |
| | GGGCGTGAGGCCCCCATCGCCTTGTGCTGGGCCCAGA |
| | GCCTCAGCTATCAGGCCTATCAGAGATGGTGGCTGGC |
| | CAGCTCAGGTTCCCCAGGAGCCAGAGGGAGGGCAGGG |
| | GTTACTAGGAAATGCGGAAAGGGTCTTTGAGGCTGGG |
| | CCCCACCCTCTCAGCTTTCACAGGAGAAACAGAGGCC |
| | CACAGGGGGCAAAGGACTTGCCAGACTCACAATGAGC |
| | CGAGCAGGTGGACTCAAGGCCCAGTGTTCGGCCCCAC |
| | AACAGCACTCACGTGCCCTTGATCGTGAGGGGCCCCC |
| | TCTCAGCCAGGCATTGAGAGCTGTGACCTGCATCTAA |
| | GATTCAGCATCAGCCATTGTGAGCTGAAGAGCCCTCA |
| | GGGTGTGCAGTCAAGGCCACAGGGCCAGACCTCCAAC |
| | GGCCAGACATCCCAGCCAGATTCCTTTCTGGTCAATG |
| | GGCGCCAGTCTGGCTTGGCTCCTGCAGGCCCAGTGGC |
| | GCCTTCTTCCCCTGGGCCTGTGGAGTCCAGCCTTTCA |
| | GTTTCCCACCCACATCCTCAGCCACAATCCAGGCTCA |
| | GAGGCAATGTCGGTGGGGAGGCCCTGTGTGACCCGTC |
| | TGTGGGTGATCCTCAGTCCTACCCTTAGCAGACAGCG |
| | CATGAGGGGCCCTCTTGAACCTGAGGGATACTCCATG |
| | TCGGAGGGGAGAAGCTGGCCTTCCCCACCCCCAGTTC |
| | CAGGCGTTGGGGAGCAGAGAAAGACCGCAGACCTGGG |
| | TCCCTTCTAACAGGCCAGGCCCGAGCCCAGCTCTCCA |
| | CCAGCCCCAGGGGCCTCGGGTCCACGCCTGGGGACTG |
| | GAGGGTGGGCCTGTCAGGCGCTGACCCAGAGGCAGGA |
| | CAGCCAAGTTCAGGATCCCAGCCAGGTGGTCCCCGTG |
| | CACCATGCAGGGGTGTGACCCACACAGGGGTGTTGCC |
| | ACCCTCACCTGACTGTGCTCATGGGCCACATGGAGGT |
| | ATCCTGGGTTCATTACTGGTCAACATACCCGTGTCCC |
| | TGCAGTGCCCCCTGTGGcgcacgcgtgcacgcgcaca |
| | cgcacacactcatacaGAGGCTCCAGCCAACAGTGCC |
| | CTGTAGTAGGCACTGCTGTCACTTCTCTAAAAGGTCG |
| | CAATCATACTTGTAAAGACCCAAGATTGTTCAGAAAT |
| | CCCAGATGGAGAAGTCTGGAAAGATCtTTTTCTCCTT |
| | TCACGGGCTGGGGAAATGTGACCTGGCCAAGGTCACA |
| | CAGCAAGTGGTGGAACCCTGGCCCCTGATTCCAGCTC |
| | ATTCCAGTTCCCAAGGCCCTGCCAGAGCCGAGAGGCT |
| | GGGCGGTCTGGGGCAGAGGAGCTGGGGTCCTGCCCCC |
| | TACACAGAGCACACAGCCCCGCAAGAGAGAAGAGACA |
| | GCTTGGGGAGAGGAATCTCCAGACCAGAGATCCCAGT |
| | ATGGGTCTCCTGTATGCTGACGGGATGGGATGTCAAG |
| | AGGGGAGGGGGGTGGGCTTTAGGGAAAGACACAAAAA |
| | TCGCTGAGAACACTGACAGGTGCGACACACCCACGGC |
| | TAATGCTAAGCTGTGGCCCATTACTCAgatct |
| Seq ID No.38 | GATCTTCTCCTAAGACCAAGGAAAACTGGTCATAGCA |
| | GGTGCACTTGTCCCCTGTGGCCATTGTCCCTCCTTCC |
| | CCAGAAGAAACAAGCACTTTCCACTCCACAAGTAGCT |
| | CCTGATCAGCTTGGAAGCCCGGTGCTGCTCTGGGCCC |
| | TGGGGACACGGCAGGGGCATCAGAGACCAAATCCTGG |
| | AACAAAGTTCCAGTGGGTGAGGCAGGCGGGACAAGCA |
| | ACACGTTATACCATAATATGAGGCAAAATATAATGTG |
| | AGTTCTTTATGAAAGGAAGGGGTTGCAGGTGCAACTG |
| | TTGGCTTAGGTGGATGGTCACCCCTGAATGGAGGAGG |
| | GGGTTCCCAGGGCATGTGCCTGGGGAGAAGGGCTCCT |
| | GGCAGGAGGGACAGCAAGTGCAAGGGCCCTGTGATCA |
| | AATGTGGCTGGGAAGTTGCAGGAACAGCTAGAAGGCC |
| | AGCAAGGTTGGAACCAAGGAAGGGGTCAGGGGAGGGG |
| | CAGGGCCGTCAGGGCCTTGCCGAGCAGCCTGAGCATC |
| | TGGAGATTTGTCCAAAGTTTCAAATGTACCTGGGCAA |
| | CCTCATGCGCATATACCATTCCTAACTTCTGCACTTA |
| | ACATCTCTAGGACTGGGACCCAGCCAGTGAAGCGGGG |
| | GGACCCAGAGAGCTCGGGTGTGAACACCGAGGTGCTG |
| | GTGGGTCTGCGTGTGTGGACATAGGGCAGTCCCGGTC |
| | CTTCCTTCACTAACACGGCCCGGGAAGCCCTGTGCCT |
| | CGCTGGTGCGCGGGTCGGCGCTTCCGGAGGGTAGAGG |
| | CCCACCTGGAGCCCGGGCAGAGTGCATGCAAGTCGGG |
| | TTCACGGCAACCTGAGCTGGCTGTGCAGGGCAGTGGG |
| | ACTCACAGCCAGGGGTACAGGGCAGACCGGTCCTGCC |
| | TCTGCGGCCCTCCCTGGCCTGTGGCCCCTGGACGTGA |
| | TCCCCAACAGTTAGCATGGCCCGCCGGTGCTGAGAAC |
| | CTGGACGAGGTCCGCAGGCGTCACTGGGCGGTCACTG |
| | AGCCCGCCCCAGGCCCCGTGTGCCCCTTCCTGGGGTG |
| | ACCGTGGAGTCCTGGATGACCCTGGACCCTAGACTTC |
| | CCAGGGTGTGTCGCGGAGGTTCCTCAGCCAGGATGTC |
| | TGCGTCTCCTCCTTCCATAGAGGGGACGGCGCGGCCT |
| | TGTGGCCAAGGAGGGGACGGTGGGTCCCGGAGCTGGG |
| | GCGGAGAACACAGGGAGCCCCTCCCAGACGCCGCTCT |
| | GGGCAGAACCTGGGAAGGGATGTGGCCATCGGGGGAT |
| | CCCTCCAGGGGATCTCCTCAGATGGGGGCTGGTCGAG |
| | TAGCTTCTGAGTCCTCCAAGGAACCGGGTCCTTCTAG |
| | TCATGACTCTGCGCAGATGAAGAAGGAGAGCACTTCT |
| | CTCCATCAGGAGGATCTGAGCTTCTCTTAATTAGAAT |
| | CAGCTCCTTGGCTTCTACCCCTTAAAAAAAGGTACAG |
| | AAACTTTGCACCTTGATCCAGTATCAGGGGAATTTAT |
| | CAATCAATGTGGGAGAAATTGGCATCTTTACCACACT |
| | GAATCTTTCAATCCATGAATATCCTCTCTCTCTTCCA |
| | TGCATAGGTTTTAATAATTCTCAATGGAGTTTAATGT |
| | AAGTTTTCCTCATAGACAATTGCCTTTGGACATCTCT |
| | TTAGACTCATCTCTAGTAAACTGATATTCTTAATGCA |
| | ATTATAAAATGTATCCTGCTTAATGTTATTTTCTATT |
| | CATTTGCTGTTATATAGAGATACAATGAGTTTCCACA |
| | TTTGAAACTGGATCTGGTAAATTGGCTACCCTTTTTT |
| | TATAGATTCTATTAATTTTTATACATTCTGTGGGACT |
| | TGCTACATACTTAATCATGTCACCTGTGAAGAATGAC |
| | AATTTGGTTGCTACCCTCCCAATTCTTATATGTCTCA |
| | TTTCTTTCCCTCTGCTGGTACTCTGGCAGCAGCAGGG |
| | AAGATAATGGGCCTCGTTATCTTGTCACAAAAGGATG |
| | TTTTTAAAGATTTCGTTATAAAACATAACGCTTTCTG |
| | GTTTTCTTTAAAGATTCTCTCACCAGCTTAAGAAAAT |
| | TTTCTTATACTCTGTATGATAAATGGGTTTTTGACAA |
| | TCATTTGTTGCATTTTACCTAGTGTTTTCTCTGCATC |
| | TTTATATGCTTTTTCTCCTTTAATCCTGAAAATTGTT |
| | TCGATTTTTCTAACATTGAACCAATCTTACATTCCTG |
| | GAATGGATGGACCAGACTAGTCCACATGTTTATTCTG |
| | CCCAATGGCTAGATTTTGTGTTCaatattttgttcag |
| | aatgtttgcatctatattcttGAGTGAGACAGAGCTG |
| | CCCTTGTTAGGTTTCACAACCGAGGTTGTGTTAGCTT |
| | CATAAAATGAGACGTTTATTCTCTAAAAGAATTGTTT |
| | CGCTTCTCTGGATGAATTTGTGTAAGGTTAGAATTGC |
| | TTACCAGTGAagatctCGGGgCCAGTTCTTCTTTAGG |
| | GGAAGATTTTCAACAATTAAGCTCAATGCCTTTAGAA |
| | GAACTGAGAGTTTCTATTATTTCTTGAGTTAAATATA |
| | TGTATTTAATTAGACTTTCTAGGAATAGTCTCATTTC |
| | ATCTCAAATAATTGACATATGCTATTAAAGCAGATTC |
| | TCATGAACCATTGTAGGTATTCCAGGTCTAGAAAAAT |
| | GTTCCCCTTTGCATCCGTAATGTGTTTAATTTTCACC |
| | TTCTTTCTTTTGTTCTTGAGAAATTCACCAAATCATT |
| | TTCAATTTCAGTCATATCCCAAAGCAACCAACTCTCT |
| | ACCTTCTTGTTTTATCATCCCTGCTGGATTTTTGTTA |
| | TCTACTTCTTCAGTATTTGTTCTTCCCTTTCTTCTAT |
| | TCCTCATTCCATTTTTCCCTTGTTTTCTAACTTTCTG |
| | AGATATATGCTTAGTTCCTTCATTTGAAGCCTTTTTA |
| | TTTTCTTTTTTTTTTTTTGGTCTTTTTGTCTTTtGTT |
| | GTTGTTGTTGTGCTATTtCTTGGGCCGCTCCCGCGGC |
| | ATATGGAGGTTCGCAGGGTAGGAGTCGAATCGGAGCT |
| | GTAGCCACCGGCCTACGGCAGAGGCACAGCAATGCGG |
| | GATCCGAGCCGCGTCTGCAACCTACACCACAGCTCAT |
| | GGCAACGCCGGATCGTTAACGCACTGAGCAAGGGCAG |
| | GAACCGAAGCCGCAACCTCATGGTTCCTAGTCGGATT |
| | CGTAACCACTGTGCCACAACAGGAACTCCGCCTTTTT |
| | ATTTTCTATAAAAATTTCTATGTACATTTTAAGGTTA |
| | TAGGTTTCCTTCTATGTACCCCATTGGCTGTATCCTC |
| | AGGGTTCTGTGGAGTGATTTCATTATTGTTCAAGTTC |
| | AATATGTCTTCTGATTTTCCAATTTGAATACCTCTCT |
| | AAATCAGTAGGTGAATATTTCTTTTTCTTTTTCTTTT |
| | CTTTTCTTCTTTTTTTTTTTCTTTCAGCCAGGTCCAT |
| | GGCATGCAGAAATTCCCAGGCCAGGAATCAAACTCTC |
| | ACCATGGCAGTGACAATGTCGGATCCTTTACCCACTA |
| | GGCCACCAGGGAACTGTGGGAGCATATGTTTTTATTT |
| | CCCGAGATCTGAGGATGGCTAGTATGTCTTCATTATT |
| | GATTTCTAGTTTGCCACTGATTTCTAGTATTTTGCTC |
| | ATAGAGTGTATGCTCAATGGTTTTGGTCATTTGAAAT |
| | GTATTTAGTCCTGCTTTATGACCCAGTATGTGGTCAG |
| | TTTTGTCAATGTTCCTTTTCTGCTTGAAGAGAACCTA |
| | CATGCTGTAACTCTGGGTGCATGTTCTGTATATAAGT |
| | CTATAGGCTGAGCCGGGGGAGCCTTCTAATCTGCCGT |
| | TATCTTCTTCGAGTTATTCTAGGTACTATTTCTTAGC |
| | CATAAACCTTTAAATTCTGATATCAATATAATGACCC |
| | CAGCCCGCTTAGGGTCGGCACTTCATGTTATCTTTTT |
| | CCATCCATTTAATCCCTCCCCACTGTTTTGGCCACAC |
| | CCGTGGGATATGGGAGTTCCTGGGCCAAGGATCaGAT |
| | CTGAGCGGCAGCTGCGACCTATGGCACAGCAgcagca |
| | atgatggatctttaacccactgcaccacactggggat |
| | tgaacccaagcctcagcagcaacccaagctactgcag |
| | agacaacaccagatccttaacctgctgtgccatagcg |
| | ggaaTTTCCATCCATTTACTTTCAAGCCAGCTGAATA |
| | ACCTAGCCCACCATGCCTGGACATGGGTGCTCTGCTT |
| | CAAATGATTTTGTTCAGTCAGCATCCATCTCTGAAAT |
| | GTGTGCCAAGCATTTATATGCATGCAAGAGTCATGTT |
| | GGCACTTCTATCATTTCCAACAGTTCAGTAGCCTTTG |
| | TATCATGACATTTCTTGGCCTTTTCTCTACAATATTT |
| | GAGGCTGAGCAGACTGGCCGTGCCCCTGTCCATGCTT |
| | CCAGAGCCTGTGTGCAGACTTCTGCTCTAGACAGAGA |
| | CAGCTAACCATCCTGCAGTGCCCAGAAAACGGAACTC |
| | AAAGACCGTGAAGTAAGGAAGGATTTATTGGCTCACG |
| | TAATCTGGAATCCAGGCATGGGGTATTCAGGGCCACC |
| | TGAACCAGAGGCGCTGGCCCTGTTCTCTAAGCTTCTT |
| | CCTGCCCTGCCCTCGTTCTGGAAGTGACCCTGAAGGA |
| | CAGCAATGAAGGGCAGGTCCCCCAGGGACAGATGACT |
| | GAGAGGTGCATTTCAAGTGCAACTTGGCCTAGATTGA |
| | GAGGCAGCAAGAAATATGGACCTACAGTGAGTCACAG |
| | GATTTACCAGTGGTTTGGCTGGGTTGTCAGTGTTACA |
| | GGCTAAACATTTGGGTCCCTCCAAAATTAACATGTTG |
| | CCACTCTAACCACCAAAATCatggtatttgggggtgg |
| | ggcccttggaggtaattaggtttagaaAGAATGAAGA |
| | GGGGGCCCTTGTGATGGGACTAGTGCCTTTATAGAGA |
| | GAGAAGAGAGAGGG |
| Seq ID No.39 | CACCTCATCCCCAACCACCTGGATGGTGGCAAGTGGC |
| | AGGCTGAGAGGCTGCATATGAGCTCATCAAGAGGGTC |
| | CCCACCCCACAGAGGCTGACCCAGCTGCCACTGCCAC |
| | GTAGTGGCTGATGGGCCAAGAGCAGGAGCCCCAGGGG |
| | CAGGTCCATTCCCTGGGGCGGCCAGGGAACCACCTGG |
| | TGGTAGGACAATTCCATTGCACCTCATCCATCAGGAA |
| | AAGGTTTGCCTTCCCTGGCAGTAATGCATCTTCCCAT |
| | AACATGGTCCCTGGCCTCTTGGAATGGCTTGGCCACC |
| | GTCATGGCCTCACCCACAAAGCCTTGTGTCTCAGCAA |
| | GGAACTTATTCCACAGCAAAGGACTTGCAGCCTGGAA |
| | TGAACTGGTCTGACTACATACCCGATTGGCCAGAAGT |
| | AGGTGGTCTATTGCAAAGTGGAGTGGGTTACCCAAGA |
| | CTCAGTTGTGCCCAAGTTGAGAGATAGCATCCTAAAA |
| | TATGGGCTTATGTCTCACTGGCTGAGGTTTATTCTTT |
| | GAATCAAAGACAATTATATGGTGTGGTCCCCCCAGAG |
| | ATAGAATACATGAGTCTGGGAATCAAGGGATAGAAGT |
| | AAGAAGAGATTTTGTCACCATTAATCCCAATAACTCG |
| | CCCAAAGAATATTTGCTTTCTGTCCTGGCAGCTCTGC |
| | TGCTTTGGCAATAACTTCCTAGAATATAATGTCTCCA |
| | CCAGGGGACTCCACAACGGTTCCATTGATTTGAAGCC |
| | AATGGGCAGAGGAGGGGCTGCCTTACTGGTCGGACTG |
| | GTCAGCCCTGATTACTAAGGAGAAATCAGGCAACTTG |
| | AACAAAACTAAGGCAGGGGGGACTTTGTCTAGAACCC |
| | AAAGCACTAAGCATCTTAGTACTTTTTAGTTCTCAGA |
| | GCCTCCAAGAACAAAGATTTAGCCCCTCAGCACCAGC |
| | AGGTAAAGAAGAGGTAAATCCAGCTGAGGACAAGAGA |
| | AATATTGAATGGATAGAGGAAGAAAGAAATTATAGAT |
| | ATCAACTATGGCCTCATGAGTAGAGTCTCCAGATTAA |
| | GCGGAATAAAAATACAGATGATTaGATCTGAACATCA |
| | GGCCAAACAAGGAAGAACAGTTTAAGTGCGACCTAGG |
| | CAATATTTGGGACATACTTATACTAAAATTTTTTCGC |
| | TATTTGAGCATCCTGTATTTTATCTGGGAACTTTATT |
| | GATCGCTAGGGAAAAAGGAACTGTGGTAACTTAGTGT |
| | ATTTTTACTTTGCTCATTATTGTGTATATACCTACTT |
| | GTATTTATCAATCATATTTACTCTGTTCTCAGTATTA |
| | CTTTATATAGCAGTTGGTGGTGATGGTTAGCAACATA |
| | TTCAGTGGAACTGTGACTGAATTTGAGGAGAAATTAA |
| | CAGAGTTGGCTGTGGCTACAATAACCCTTCGGGACAT |
| | GTGTCCCCTCATTTTGGGGAGATGGTTagatctGTGG |
| | GTAAATGTTAGGGCATCTGAGCCAGAAAGCAAGATTT |
| | TGCCAGCTGGTGCAATGTCAGATTTTACCAGCAGAGG |
| | GTGCCAGAGGAATGCGGCAAAACCCGAGTGCCAGAAA |
| | GCACCTCCCTGTTTTCCAGCTTTTCTTCCTTTTTATT |
| | TATTTTATTTACGGCCCAGGAGTCCGTAATAGCGCTG |
| | AGGATGGCCCAGGCTCTTCTCAGCAGCCCTGACTGAC |
| | TAGTTCAGCAATGCGCTCAGGCCCCATCTGGCCACCG |
| | GGCAGCCTCTTCTGTGGTAGCTCCAGCCTCAGCCAGT |
| | GCAAAAGGCTACCCTACACTGGCGCCACTTCTACAAT |
| | CAGCACTGGCCACACCCTCCACGCCATCCGGCACGGA |
| | GCCAGGTGATCTGCCGGGCAGATTGCAGTTCGTGCTG |
| | CCTGAGTCCAGGTGATTACACTGGGTGCATCTTTTCT |
| | TTCTGGACGAtTCattccattttttt |

Bovine Lambda Light Chain

In a further embodiment, nucleic acid sequences are provided that encode bovine lambda light chain locus, which can include at least one joining region-constant region pair and/or at least one variable region, for example, as represented by Seq ID No. 31. In Seq ID No 31, bovine lambda C can be found at residues 993-1333, a J to C pair can be found at the complement of residues 33848-35628 where C is the complement of 33848-34328 and J is the complement of 35599-35628, V regions can be found at (or in the complement of) residues 10676-10728, 11092-11446, 15088-15381. 25239-25528, 29784-30228, and 51718-52357. Seq ID No. 31 can be found in Genbank ACCESSION No. ACI 17274. Further provided are vectors and/or targetting constructs that contain all or part of Seq ID No. 31, for example at least 100, 250, 500, 1000, 2000, 5000, 10000, 20000, 500000, 75000 or 100000 contiguouos nucleotides of Seq ID No. 31, as well as ceels and animals that contain a disrupted bovine lambda gene.

| | | |
|---|---|---|
| Seq ID No 31 | 1 | tgggttctat gccacccagc ttggtctctg atggtcactt gaggccccca tctcatggca |
| | 61 | aagagggaac tggattgcag atgagggacc gtgggcagac atcagaggga cacagaaccc |
| | 121 | tcaaggctgg ggaccagagt cagagggcca ggaagggctg gggaccttgg gtctagggat |
| | 181 | ccgggtcagg gactcggcaa aggtggaggg ctccccaagg cctccatggg gcggacctgc |
| | 241 | agatcctggg ccggccaggg acccagggaa agtgcaaggg gaagacgggg gaggagaagg |
| | 301 | tgctgaactc agaactgggg aaagagatag gaggtcagga tgcaggggac acggactcct |
| | 361 | gagtctgcag gacacactcc tcagaagcag gagtccctga agaagcagag agacaggtac |
| | 421 | cagggcagga aacctccaga cccaagaaga ctcagagagg aacctgagct cagatctgcg |
| | 481 | gatgggggga ccgaggacag gcagacaggc tccccctcga ccagcacaga ggctccaagg |
| | 541 | gacacagact tggagaccaa cggacgcctt cgggcaaagg ctcgaacaca catgtcagct |
| | 601 | caaaatatac ctggactgac tcacaggagg ccagggaggc cacatcatcc actcagggga |
| | 661 | cagactgcca gccccaggca gaccccatca accgtcagac gggcaggcaa ggagagtgag |
| | 721 | ggtcagatgt ctgtgtggga aaccaagaac cagggagtct caggacagcg ctggcagggg |
| | 781 | tccaggctca ggctttccca ggaagatggg gaggtgcctg agaaaacccc acccaccttc |
| | 841 | cctggcacag gccctctggc tcacagtggt gcctggactc ggggtcctgc tgggctctca |
| | 901 | aaggatcctg tgtccccctg tgacacagac tcaggggctc ccatgacggg caccagacct |
| | 961 | ctgattgtgg tcttcttccc ctcgcccact ttgcaggtca gcccaagtcc acaccctcgg |
| | 1021 | tcaccctgtt cccgccctcc aaggaggagc tcagcaccaa caaggccacc ctggtgtgtc |
| | 1081 | tcatcagcga cttctacccg ggtagcgtga ccgtggtcta gaaggcagac ggcagcacca |
| | 1141 | tcacccgcaa cgtggagacc acccgggcct ccaaacagag caacagcaag tacgcggcca |
| | 1201 | gcagctacct gagcctgatg ggcagcgact ggaaatcgaa aggcagttac agctgcgagg |
| | 1261 | tcacgcacga ggggagcacc gtgacgaaga cagtgaagcc tcagagtgtt cttagggccc |
| | 1321 | tgggccccca ccccggaaag ttctaccctc ccaccctggt tccccctagc ccttcctcct |
| | 1381 | gcacacaatc agctcttaat aaaatgtcct cattgtcatt cagaaatgaa tgctctctgc |
| | 1441 | tcatttttgt tgatacattt ggtgccctga gctcagttat cttcaaagga aacaaatcct |
| | 1501 | cttagccttt gggaatcagg agagagggtg gaagcttggg ggtttgggga gggatgattt |
| | 1561 | cactgtcatc cagaatcccc cagagaacat tctggaacag gggatggggc cactgcagga |
| | 1621 | gtggaagtct gtccaccctc cccatcagcc gccatgcttc ctcctctgtg tggaccgtgt |
| | 1681 | ccagctctga tggtcacggc aacacactct ggttgccacg ggcccagggc agtatctcgg |
| | 1741 | ctccctccac tgggtgctca gcaatcacat ctggaagctg ctcctgctca agcggccctc |
| | 1801 | tgtccactta gatgatgacc cccctgaagt catgcgtgtt ttggctgaaa ccccaccctg |
| | 1861 | gtgattccca gtcgtcacag ccaagactcc ccccgactcg acctttccaa gggcactacc |
| | 1921 | ctctgcccct cccccagggc tccccctcac agtcttcagg ggaccggcaa gcccccaacc |
| | 1981 | ctggtcactc atctcacagt tcccccaggt cgccctcctc ccacttgcat ggcaggaggg |
| | 2041 | tcccagctga cttcgaggtc tctgaccagc ccagctctgc tctgcgaccc cttaaaactc |
| | 2101 | agcccaccac ggagcccagc accatctcag gtccaagtgg ccgttttggt tgatgggttc |
| | 2161 | cgtgagctca agcccagaat caggttaggg aggtcgtggc gtggtcatct ctgaccttgg |
| | 2221 | gtggtttctt aggagctcag aatgggagct gatacacgga taggctgtgc taggcactcc |
| | 2281 | cacgggacca cacgtgagca ccgttagaca cacacacaca cacacacaca cacacacaca |
| | 2341 | cacacacgag tcactacaaa cacggccatg ttggttggac gcatctctag gaccagaggc |
| | 2401 | gcttccagaa tccgccatgg cctcactctg cggagaccac agctccatcc cctccgggct |
| | 2461 | gaaaaccgtc tcctcaccct cccaccgggg tgacccccaa agctgctcac gaggagcccc |
| | 2521 | cacctcctcc aggagaagtt ccctgggacc cggtgtgaca cccagccgtc cctcctgccc |
| | 2581 | ctcccccgcc tggagatggc cggcgcccca tttcccaggg gtgaactcac aggacgggag |
| | 2641 | gggtcgctcc cctcacccgc ccggagggtc aaccagcccc tttgaccagg aggggggcgg |
| | 2701 | acctggggct ccgagtgcag ctgcaggcgg gcccccgggg gtggcggggc tggcggcagg |
| | 2761 | gtttatgctg gaggctgtgt cactgtgcgt gtttgctcgg tggagggacc cagctggcca |
| | 2821 | tccggggtga gtctcccctt tccagctttc cggagtcagg agtgacaaat gggtagattc |
| | 2881 | ttgtgttttt cttacccatc tggggctgag gtctccgtca ccctaggcct gtaaccctcc |
| | 2941 | cccttttagc ctgttccctc tgggcttctt cacgtttcct tgagggacag tttcactgtc |
| | 3001 | acccagcaaa gcccagagaa tatccagatg gggcaggcaa tatgggacgg caagctagtc |
| | 3061 | caccctctta ccttgggctc cccgcggcct ccggataatg tctgagctgc ctccctggat |
| | 3121 | gcttcacctt ctgagactgt gaggcaagaa accccctccc caaaagggag gagacccgac |
| | 3181 | cccagtgcag atgaacgtgc tgtgagggga ccctgggagt aagtggggtc tggcggggac |
| | 3241 | cgtgatcatt gcagactgat gccccaggca gggtgagagg tcatggccgc cgacaccagc |
| | 3301 | agctgcaggg agcacaggcc gggggcaagt catgcagaca ggacaggacg tgtgaccctg |
| | 3361 | aagagtcaga gtgacacgcg gggggggggc ccggagctcc cgagattagg gcttgggtcc |
| | 3421 | taacgggatc caggagggtc cacgggccca ccccagccct ctccctgcac ccaatcaact |
| | 3481 | tgcaataaaa cgtcctctat tgtcttacaa aaaccctgct ctctgctcat gtttttcctt |
| | 3541 | gccccgcatt taatcgtcaa cctctccagg attctggaac tggggtgggg nnnnnnnnnn |
| | 3601 | nnnnnnnnnn nnnnnnnnnn nnnnnnnnnn nnnnnnnnnn nnnnnnnnnn nnnnnnnnnn |
| | 3661 | nnnnnnnnnn nnnnnnnnnn nnnnnnnnnn agcttatgtg gtgggcaggg gggtagtaag |
| | 3721 | atcaaaagtg cttaaattaa taaagccggc atgatatacg agtttggata aaaaatagat |
| | 3781 | ggaaaagtaa gaaaggacag gaggggggtg aggcggaaga aagggggaag aaggaaaaaa |
| | 3841 | aaataagaga gaggaacaaa gaaagggagg ggggccggtg atgggggtgg gatagaatat |
| | 3901 | aataattgga gtaaagagta gcgggtggct gttaattccg ggggggaata gagaaaaaaa |
| | 3961 | aaaaaaaatg tgcgggtggg cggtaagtat ggagatttta taaatattat gtgtggaata |
| | 4021 | atgagcgggg gtggacgggc aaggcgagag taaaaagggg cgagagaaaa aaattaggat |
| | 4081 | ggaatatatg gggtaaattt taaatagagg gtgatatatg ttagattgag caagatataa |
| | 4141 | atatagatgg tgggggaaaa gagacaaggg tgagcgccaa aacgccctcc cgtatcattt |
| | 4201 | gccttccttc ctttaccacc tcgttcaaac tctttttcga gaaccctgaa gcggtcaggc |
| | 4261 | ccggggctgg gggtgggata cccggggagg ggctgcgcct cctcctttgc agagggggtc |
| | 4321 | gaggagtggg agctgaggca ggagactggc aggctggaga gatggctgtt gacttcctgc |
| | 4381 | ctgtttgaac tcacagtcac agtgccagac ccactgaatt gggctaaata ccatattttt |
| | 4441 | ctggggagag agtgtagagc gagcgactga ggcgagctca tgtcatctac agggccgcca |
| | 4501 | gctgcaggga ctttgtgtgt gtcgtgctcg ttgctcagtt gtgtccgact ctttatgact |
| | 4561 | tcatggactg taacctgcca ggctcctctg tccgtggaat tctccaggca agaatactgg |
| | 4621 | agtgggtagc cattctcatc tccgggggat cttcctgacc caagaatcaa acctgagtct |
| | 4681 | cccgcattgc aggcagcttc tttcttgtct gagccaccag ggaagcccct taagtggagg |
| | 4741 | atctaaatag agtgtttagg agtataagag aaaggaagga cgtctataca agatccttcg |
| | 4801 | gttcctgtaa ctacgactcg agttaacaag ccctgtgtga gtgagttgcc agtaattatt |
| | 4861 | gctaacctgt ttctttcact cactgagcca ggtatcctgt gagacggcat acttacctcc |
| | 4921 | tcttctgcat tcctcgggat ggagctgtgc ggtggcctct aggactacca catcgaccag |
| | 4981 | gtcagaccca gggacagagg attgctgaga tgcactgaga agtttgtcag cctaggtctt |
| | 5041 | cacccacaca gactgtgctg tcgtctacca cgtaattctt cctgtccaaa gaactggtta |
| | 5101 | aacgctcctg aagcgtattc tggtctgctt caaaaagtgc ctctttcctt tataagttcc |
| | 5161 | gccaatcctg gactttgtcc caggccagtc tactttattt gtgggaaagg tttttttggt |
| | 5221 | cttttttgtt ttaaactctg cagaaattgc ttacactttt ggtgtgcaat ggctcactct |
| | 5281 | tacggttcta gctgtattca aaggggttgc ttttctttgt ttttaaagct ttttgaacgt |
| | 5341 | ggaccatttt taaagtcttt attaaacgtc taacatcgtt tctggtttat tttctggtgg |
| | 5401 | tctggccatg aggcctacgg gtcttagctc ccctaccagg gtccaaccca catcccttgc |
| | 5461 | actggacggc aaggtcttaa cctttgaacc accagagagc ttctgaaagg ggctgctttt |
| | 5521 | ctccaatcct ctttgctccc tgcctgctgg tagggattca gcacccctgc aatagccctg |
| | 5581 | tctgttctta ggggctcagt agcctttctg cctgggtgtg gagctggggt tgtaagagag |
| | 5641 | cttcatggat ttggacacga cctacgactc agaggtaaga ctccatctta gcgctgtaat |
| | 5701 | gacctctttc caacaaccac ccccaccacc ctggaccact gatcaggaga gatgattctc |
| | 5761 | tctcttatca tcaacgtggt cagtcccaaa cttgcacccg gcctgtcata gatgtagcag |
| | 5821 | gtaagcaata aatatttgtt gaatgttaag tgaattgaaa taacataagt gaaaaagaaa |
| | 5881 | acacttaaaa acatgtgttt ttataattac acagtaaaca tataatcatt gtagaaaaaa |
| | 5941 | atcgaaagag tggcgggggc caagtgaaaa ccaccatccc tggtatgtcc acccgcccgg |
| | 6001 | gtagccccag gtaagaggtg cggacacgga tggccctgta gacacagaga cacacgctca |
| | 6061 | tatgctgggt cttgtcttgt gacctcttgg ggatgatgtt attttcacga tgccattcaa |
| | 6121 | accttctacc acaccatttt tagagggtcg ttcatcgtaa atcagttcac tgctttgttt |
| | 6181 | tctgatrttg aaagtgtcac attcttcgag aaatgagaag gaacaggcgc gcataaggaa |
| | 6241 | gaaagtaaac acgtggcctt gcttccaggg ggcactcagc gtgttggtgt gcacgctggc |
| | 6301 | agtcttttct ctgtgacagt catggccttt tcccaaaggt gggctcagat aagaccgcct |
| | 6361 | cccatcccct gtccctgtcc ccgtccccta cggtggaacc cacccacggc acgtctccga |
| | 6421 | ggccctttgg ggctgtggac gttaggctgt gtggacatgc tgctggtggg gacccagggc |
| | 6481 | tgggcagcac gttgtccctg ggtcccgggc cagigaggag ctcccaagga gcagggctgc |
| | 6541 | tgggccaaag ggcagtgcgt cccgaggcca tggacaaggg gatacatttc ctgctgaagg |
| | 6601 | gctggactgc gtctccctgg ggccccttgg agtcatgggc agtggggagg cctctgctca |
| | 6661 | ccccgttgcc cacccatggc tcagtctgca gccaggagcg cctggggctg ggacgccgag |
| | 6721 | gccggagccc ctccctgctg tgctgacggg ctcggtgacc ctgccgcccc ctccctgggg |
| | 6781 | ccctgctgac cgcgggggcc accccggcca gttctgagat tcccctgggg tccagccctc |
| | 6841 | caggatccca ggacccagga tggcaaggat gttgaggagg cagctagggg gcagcatcag |
| | 6901 | gcccagaccg gggctgggca ggggctgggc gcaggcgggt gggggggtct gcacnccccc |
| | 6961 | acctgcnagc tgcncnnncn tttgntnncg tcctccctgn tcctggtctg tcccgcccgg |
| | 7021 | ggggcccccc ctggtcttgt ttgftccccc tccccgtccc ftcccccctt tttccgtcct |
| | 7081 | cctcccttct tttattcgcc ccttgtggtc gttttttttc cgtccctctt ttgttttttt |
| | 7141 | gtctttttct ttttccccct cttctccctt gctctctttt tcattcgtcg gtttttctgc |
| | 7201 | tcccttccct ctcccccccg ctttttttcc ctgtctgctt tttgtgttct ccctctctac |
| | 7261 | cccccctgca gcctattttt tttatatatc catttccccc tagtatttgg cccccgctta |
| | 7321 | cttctcccta atttttattt tcctttcttt aactaaaatc accgtgtggt tataagtttt |
| | 7381 | aacctttttt gcaccgccca caatgcaatc ttcacgcacg ccccccccgt cagcctcctt |
| | 7441 | aaataccttt gcctactgcc cccctccttg tataataacg cgtcacgtgg tcaaccatta |
| | 7501 | tcacctctcc accaccttac cacattttcc ttcnnnnnnn nnnnnnnnnn nnnnnnnnnn |
| | 7561 | nnnnnnnnnn nnnnnnnnnn nnnnnnnnnn nnnnnnnnnn nnnnnnnnnn nnnnnnnnnn |
| | 7621 | nnnnnnnnnn nnntgaaaaa agaaaaggct gggcaggttt taatatgggg gggttggagt |
| | 7681 | ggaatgaaaa tgcattggag tggttgcaac aaatggaaag gtctcaggag cgctcctccc |
| | 7741 | ccatcaggag ctggaaagaa gtggaagcaa agcaaggaat tcgtgtgatg gccagaggtc |
| | 7801 | aggggcaggg agctgcaaag actgccggct gtttgtgact gnccgtctcc gggtgcattt |
| | 7861 | gttagcaggg aggcattaca ctcatgtctt ggtttgctaa ctaattctta ctattgttta |
| | 7921 | gttgcaaggt catgtctgac tctttgcaac ccagggactg cagcccgcca ggctcctctg |
| | 7981 | tccatgggat ttcgcaggca agaatactgg aggtggtagc cattttcttc accatgggat |
| | 8041 | cttcccgagc cagaaatgga acccgagtcg cctcctgtgc atggggtctg ctgcctaaca |
| | 8101 | ggcagatatt tgacgtctga gccaacaggg aggacagacg gtaattatac caaccattga |
| | 8161 | aagaggaatt acacactaat ctttatcaaa atctttcaaa cagtagagga gaaaggatac |
| | 8221 | tctctagttt attccataaa gttggaatta cgcttatcaa taaagacatt acaagaaaag |
| | 8281 | aaagtgaagc cccaaatgcc ttataaatat acaagaaaaa atcttttaag atattagcca |
| | 8341 | acttaatcaa caaaaaatgt atcaaaagtc caagtaacat tcaccccagg aatgcaagtg |
| | 8401 | tggttcagcc taagacaatc agtcatgagt ataccacgga aacaaattaa agagaaaaga |
| | 8461 | cattaaatct cacaaatggt gcagaaaaag atttggcaat atcgaacatc ttttcatgac |
| | 8521 | caaaggaaaa aaaagaaaca aaacaccaga aaattctgtg tagaaagaat atatctcaac |
| | 8581 | ccaatgaagg gcatttatga aaaacccaca gcatacatca cactccatga gaaagactga |
| | 8641 | aagctttccc cactgccatt gaactctgtc ctggaaattc tagtcacagc gacagaacaa |
| | 8701 | gagaaagaaa taacggccgt ctaaactggt aggaagaaat caaagcgtct ctattctctg |
| | 8761 | ggcgcataat acaatataga caaatttcta aagtccacaa aaattcctag agctcataat |
| | 8821 | gaatccagaa atgcgtcagg gctcaagatt cagatgcaaa aatcgtctgg gttttgatgc |
| | 8881 | accaacaaac aattccatta acaataatac caaggaatta atttaactta gaagagaaaa |
| | 8941 | gacctgttta cagagagtta taaaacattt ggtgatgaaa ttaaataaga gtaaatcata |
| | 9001 | tagaaacacc gttcgtgttt tggagaccta atgtcataaa cgtggcaaca cagagacgcc |
| | 9061 | tcacggggaa ccctgagcct ccttctccaa acaggcctgc tcatcatttc acaggtaacc |
| | 9121 | tgagacccta aagcttgact ctgaggcact ttgagggcat gaagagagca gtagctcctc |
| | 9181 | ccatgggacc gacagtcaag gcccagggaa tgaccacctg gacagatgac ttcccggcct |
| | 9241 | catcagcagt cggtgcagag tggccaccag ggggcagcag agagtcgctc aacactgcac |
| | 9301 | ctggagatga ggcaacctgg gcatcaggtg cccatgcagg ggctggatac ccacacctca |
| | 9361 | cacctgagga caggggccgg ctttctgtgg tgtcgccctc tcaggatgca cagactccac |
| | 9421 | cctcttcgct tgcattgaca gcctctgtcc ttcctggagg acaagctcca ccttccccat |
| | 9481 | ctctccccag ggggctgggg ccaacagtgt tctctcttgt ccactccagg aacacagagc |
| | 9541 | caagagattt atttgtctta attagaaaaa ctatttgtat tcctgcattt ccccagtaac |
| | 9601 | tgaaggcaac tttaaaaaat gtatttcctg gacttccctg gtgggccagt ggctagactc |
| | 9661 | tgagctccca gtgcatgggg cctgggttca atccctgctc aggaaactac atcccacagg |
| | 9721 | ctgcaaataa gatcctgcat gccacccgat gcaggcaaag aaacaagtgt tcggtatgca |
| | 9781 | tgtatttcac gtgaggtgtt tctataattt acagccagta ttctgtctta cacttagtca |
| | 9841 | ttcctttgag cacatgatcg gtcgatggcc cagaccacac acaggaatac tgaggcccag |
| | 9901 | cacccaccgg ctgcccagaa cctcatggcc aagggtggac acttacagga cctcagggga |
| | 9961 | cctttaagaa cgccccgtgc tcttggcagc ggagcagtgt taagcatggc tctgtccctc |
| | 10021 | gggagctgtg tctgggctgc gtgcatcacc tgtggtgtgg gcctggtgag ggtcaccgtc |
| | 10081 | caggggccct cgagggtcag aagaaccttc ccttaaaagt tctagaggtg gagctagaac |
| | 10141 | cagacccaca tgtgaactgc acccaaaaac agtgaaggat gagacacttc aaagtcctgg |
| | 10201 | gtgaaattaa gggccttccc ctgaaccagg atggagcaga ggaaggactt ggcttccagg |
| | 10261 | aaaccctgac gtctccaccg tgactctggc cggggtcatg gcagggccca ggatcctttg |
| | 10321 | gtgcaaagga ctcagggttc ctggaaaata cagtctccac ctctgagccc tcagtgagaa |
| | 10381 | gggcttctct cccaggagtg gggcaaggac ccagattggg gtggagctgt ccccccagac |
| | 10441 | cctgagacca gcaggtgcag gagcagcccc gggctgaggg gagtgtgagg gacgttcccc |
| | 10501 | ccgctctcaa ccgctgtagc cctgggctga gcctctccga ccacggctgc aggcagcccc |
| | 10561 | caccccaccc cccgaccctg gctcggactg atttgtatcc ccagcagcaa ggggataaga |
| | 10621 | caggcctggg aggagccctg cccagcctgg gtttggcgag cagactcagg gcgcctccac |
| | 10681 | catggcctgg accccctcct cctcggcctc ctggctcact gcacaggtga gccccagggt |
| | 10741 | ccacccaccc cagcccagaa ctcggggaca ggcctggccc tgactctgag ctcagtggga |
| | 10801 | tctgcccgtg agggcaggag gctcctgggg ctgctgcagg gtgggcagct ggaggggctg |
| | 10861 | aaatccccct ctgtgctcac tgctaggtca gccctgaggg ctgtgcctgc cagggaaagg |
| | 10921 | ggggtctcct ttactcagag actccatcca ccaggcacat gagccggggg tgctgagact |
| | 10981 | gacggggagg gtgtccctgg gggccagaga atctttggca cttaatctgc atcaggcagg |
| | 11041 | gggcttctgt tcctaggttc ttcacgtcca gctacctctc ctttcctctc ctgcaggcgc |
| | 11101 | tgtgtcctcc tacgagctga ctcagtcacc cccggcatcg atgtccccag gacagacggc |
| | 11161 | caggatcacg tgttgggggc ccagcgttgg aggtganaat gttgagtggc accagcagaa |
| | 11221 | gccaggccag gcctgtgcgc tggtctccta tggtgacgat aaccgaccca cgggggtccc |
| | 11281 | tgaccagttc tctggcgcca actcagggaa catggccacc ctgcccatca gcggggcccg |
| | 11341 | ggccaaggat gaggccgact attactgtca gctgtgggac agcagcagta acaatcctca |
| | 11401 | cagtgacaca ggcagacggg aagggagatg caaaccccct gcctggcccg cgcggcccag |
| | 11461 | cctcctcgga gcagctgcag gtcccgctga ggcccggtgc cctctgtgct cagggcctct |
| | 11521 | gttcatcttg ctgagcagcg gcaagtgggc attggttcca agtcctgggg gcatatcagc |
| | 11581 | acccttgagc cagagggtta ggggttaggg ttagggttag gctgtcctga gtcctaggac |
| | 11641 | agccgtgtcc cctgtccatg ctcagcttct ctcaggactg gtgggaagat tccagaacca |
| | 11701 | ggcaggaaac cgtcagtcgc ttgtggccgc tgagtcaggc agccattctg gtcagcctac |
| | 11761 | cggatcgtcc agcactgaga cccggggcct ccctggaggg caggaggtgg gactgcagcc |
| | 11821 | cggcccccac accgtcaccc caaaccctcg gagaaccgcg ctccccagga cgcctgcccc |
| | 11881 | tttgcaacct gacatccgaa cattttcatc agaacttctg caaaatattc acaccgctcc |
| | 11941 | tttatgcaca ttcctcagaa gctaaaagtt atcatggctt gctaaccact ctccttaaat |
| | 12001 | attcttctct aacgtccatc ttccctgctc cttagacgcg ttttcattcc acatgtctta |
| | 12061 | ctgcctttgg tctgctcgtg tattttcttt tttttttttt ttttattgga atatatttgc |
| | 12121 | gttacaatgt tgaatttgaa ttggtttctg ttgtacaaca atgtgaatta gttatacatg |
| | 12181 | tcctgaggag gggcggctgc gtgggtgcag gagggccgag aggagctact ccacgttcaa |
| | 12241 | ggtcaggagg ggcggccgtg aggagatacc cctcgtccaa ggtaagagaa acccaagtaa |
| | 12301 | gacggtaggt gttgcgagag ggcatcagag ggcagacaca ctgaaaccat aatcacagaa |
| | 12361 | actagccaat gtgatcacac ggaccacagc ctggtctaac tcagtgaaac taagccatgc |
| | 12421 | ccatggggcc aaccaagatg ggcgggtcat gtgcccatgg ggccaaccaa gatgggcggg |
| | 12481 | tcatggtgaa gaggtctgat ggaatgtggt ccactggaga agggaaaggc aaaccacttc |
| | 12541 | agtattcttg ccttgagagc cccatgaaca gtatgaaaag gcaaaatgat aggatactga |
| | 12601 | aagaggaact ccccaggtca gtaggtgccc aatatgctac tggagatcag tggagaaata |
| | 12661 | actccagaaa gaatgaaggg atggagccaa agcaaaaaca atacccagtt gtggatgtga |
| | 12721 | ctggtgatag aagcaagggc caatgatgta aagagcaata ttgcatagga acctggaatg |
| | 12781 | ttaagtccaa gannnnnnnn nnnnnnnnnn nnnnnnnnnn nnnnnnnnnn nnnnnnnnnn |
| | 12841 | nnnnnnnnnn nnnnnnnnnn nnnnnnnnnn nnnnnnnnnn nnnnnnnnnn nnagaatttt |
| | 12901 | gagcattact ttactagcgt gtgagacgag tgcaattgtg cggtagtttg agcattcttt |
| | 12961 | ggcattgcct ttctttggga ttggaatgaa aactgacctg ttccaggcct gtggccactg |
| | 13021 | ctgagttttc caaatttgct ggcgtattga gtgcatcact ttaacagcat catcttttag |
| | 13081 | gatttgaaat agctcaactg gaattctatc actttagcta attccattca ttagctttgt |
| | 13141 | ttgtagtgat gcttcctaag gcccccctgg ctttatcttc ctggatgtct ggctctggtg |
| | 13201 | agtgatcaca ccgctgtgat tatctgggtc atgaaggtct ttttgtatag ttcttcttag |
| | 13261 | gaacagatat tatgatctcc atccttgcat ctcgttatat ctagagaagc actgactccc |
| | 13321 | ttcatggtga cgtcagatcc tcatgactaa caaatggcct tttgtaagat gagtgcctca |
| | 13381 | tggtattgag ctcccccgtc accaagacct tatgactgac ctcccccact gccccaggtg |
| | 13441 | cctctcgaag cgtctgagat gccgcctccc aggctgcact cctcattttg cccccaataa |
| | 13501 | aacttaactt gcagctctcc agctgtgcat ctgtgtttag ttgacagtac aaatataatg |
| | 13561 | gaaaatttaa attaaatata atctatgggg agaaatccaa acatcttatg agggagagag |
| | 13621 | agggagagaa aggaaagaag aagaagcagg aggaggagga gagtagagaa acagggggag |
| | 13681 | ggcggcaggg agacagaggg gaggacaccg aggggaaagg gaggaaggcg agtgcagtga |
| | 13741 | gagagaggcc agagttcatc agagtctgga ctcgcagccc aatcccacgg gtgtgtcccg |
| | 13801 | aagcagggga gagcctgagc caggcggaga cagagctgtg tctccagtcc tcgtggccgt |
| | 13861 | gacctggagc tgtgtggtca gcccccctga ccccagcctg gccctgctgg tggtcggagg |
| | 13921 | cagtgatcct ggacacagtg tctgagcgtc tgtctgaaat ccctgtggag gcgccactca |
| | 13981 | ggacggacct cgcctggccc cacctggatc tgcaggtcca ggcccgagtg gggcttcctg |
| | 14041 | cctggaactg agcagctgga ggggcgtctg caccccagca gtggagcggc cccaggggcg |
| | 14101 | ctcagagctg ccggggggac acagagcttg tctgagaccc agggctcgtc tccgaggggt |
| | 14161 | cccctaaggt gtcttctggc cagggtcaga gccgggatga gcacaggtct gagtcagact |
| | 14221 | ttcagagctg gtggctgcat ccctggggac agagggctgg gtcctaacct gggggtcaga |
| | 14281 | gggcaggacg ggagcccagc tgacccctgg ggactggcct cctctgtggt ctcccctggg |
| | 14341 | cagtcacagc ttccccggac gtggactctg aggaggacag ctggggcctg gctgtcagga |
| | 14401 | gggggttcga gaggccacac tcagaggagg agaccctggc ctgcttgggt tgtgactgag |
| | 14461 | tttttggggt cctctaggag actctggccc tgcaggccct gcaaggtcat ctctagtgga |
| | 14521 | gcaggactcc acaagattga tgaactgaat cctctaggag aggtgtggtt gtgagggggc |
| | 14581 | agcattctag aaccaacagc gtgtgcaggt agctggcacc gggtctagtg gcggcgggca |
| | 14641 | gggcactcag ggccgactag gggtctgggg gattcaatgg tgcccacagc actgggtctt |
| | 14701 | ccatcagaat cccagacttc acaaggcagt ttcggggatt aggtcaggac gtgagggcca |
| | 14761 | cagagaggtg gtgatggcct agacaagtcc ttcacagaga gagctccagg ggccatgata |
| | 14821 | agatggatgg gtctgtattg tcagtttccc cacatcaaca ccgtggtccc gccagcccat |
| | 14881 | aatgctctgt ggatgcccct gtgcagagcc tacctggagg cccgggaggc ggggccgcct |
| | 14941 | gggggctcag ctccggggta accgggccag gcctgtccct gctgtgtcca cagtcctccc |
| | 15001 | ggggttggag gagagtgtga gcaggacagg agggtttgtg tctcacttcc ctggctgtct |
| | 15061 | gtgtcactgg gaacattgta actgccactg gcccacgaca gacagtaata gtcggcttca |
| | 15121 | tcctcggcac ggaccccact gatggtcaag atggctgttt tgccggagct ggagccagag |
| | 15181 | aactggtcag ggatccctga gcgccgctta ctgtctttat aaatgaccag cttaggggcc |
| | 15241 | tggcccggct tctgctggta ccactgagta tattgttcat ccagcagctc ccccgagcag |
| | 15301 | gtgatcttgg ccgtctgtcc caaggccact gacactgaag tcaactgtgt cagttcatag |
| | 15361 | gagaccacgg agcctggaag agaggaggga gaggggatga gaaggaagga ctccttcccc |
| | 15421 | aagtgagaag ggcgcctccc ctgaggttgt gtctgggctg agctctgggt ttgaggcagg |
| | 15481 | ctcagtcctg agtgctgggg gaccagggcc ggggtgcagt gctggggggc cgcacctgtg |
| | 15541 | cagagagtga ggaggggcag caggagaggg gtccaggcca tggtggacgt gccccgagct |
| | 15601 | ctgcctctga gcccccagca gtgctgggct ctctgagacc ctttattccc tctcagagct |
| | 15661 | ttgcaggggc cagtgagggt ttgggtttat gcaaattcac cccccggggg cccctcactc |
| | 15721 | agaggcgggg tcaccacacc atcagccctg tctgtcccca gcttcctcct cggcttctca |
| | 15781 | cgtctgcaca tcagacttgt cctcagggac tgaggtcact gtcaccttcc ctgtgtctga |
| | 15841 | ccacatgacc actgtcccaa gcccccctgc ctgtggtcct gggctcccca gtggggcggt |
| | 15901 | cagcttggca gcgtcctggc cgtggactgc ggcatggtgt cctggggttc actgtgtatg |
| | 15961 | tgaccctcag aggtggtcac tagttctgag gggatggcct gtccagtcct gacttcctgc |
| | 16021 | caagcgctgc tccctggaca cctgtggacg cacagggctg gttcccctga agccccgctt |
| | 16081 | gggcagccca gcctctgacc tgctgctcct ggccgcgctc tgctgccccc tgctggctac |
| | 16141 | cccatgtgct gcctctagca gagctgtgat ttctcagcat aactgattac tgtctccagt |
| | 16201 | actttcatgt ccctgtgacg ggctgagtta gcatttctca cactagagaa ccacagtcct |
| | 16261 | cctgtgtaaa gtgatcacac tcctctctgt gggacttttg taaaagattc tgcagccagg |
| | 16321 | agtcatgggt ggtcttagct gagaaatgct ggatcagaga gacctgataa ccgatgtgaa |
| | 16381 | gaggggaacc tggaagatct tcagttcagt tcatttcagt cattcagttg tgtccgactg |
| | 16441 | tttgggatcc catggactgc cacacgccag tcctccctgt ccatcaccaa cttctgaagc |
| | 16501 | ttgttcaaac tcatgtccat caagttggag atgcctttca accatctcat cctctgtcat |
| | 16561 | ccccttctcc tcccgccttc aatcttccct agcattaggg tcttttccgt gagtcagttc |
| | 16621 | ttcgcatcag gtggccaagt tttggagttt cagtttcagc atcagtcctt tcaatgaata |
| | 16681 | gtaaggactg atttccttta ggatggactg gtttgatatc cttgcagttc aagggactct |
| | 16741 | caagagtctt ctccaacact gcagttaaaa gccatcaatt cttcggtgct cagctttctt |
| | 16801 | tttggtacaa ctctcacatt catacatgac taccgaaaat acattagtcg tgtagaacca |
| | 16861 | gtttggggct tcccacgtgg ctctagtggt aaagaatatg cctgccaact cagaagatgt |
| | 16921 | aagagatgcg gttcaatctc tgggtcggga agatcccctg gagaagggca tgacaaccca |
| | 16981 | ctccagtatt tttgcctgga gaatcccatg gacagagaag cctggtggac tgcagtccat |
| | 17041 | ggagtctcac agagtcagac acgactgaag caacttagct acttggaaaa gagcatgcac |
| | 17101 | gaagctgtct aaaaaacagg tcaagaagtc ttgtgttttg aaggtttact gagaaagttg |
| | 17161 | atgcactgct ccaacacttc ctctcagttg aaaagatcag aagcgttaga tcaaatggtg |
| | 17221 | gtcaatacct tggatgcgct ccaacaggtt atatctgcag atggaaatga aggcagttta |
| | 17281 | tggggtaact ggaggacaag atgagatcat acacttggaa cactgtctgg catcaaaggc |
| | 17341 | gtgtacagta aacattagct gttattagca aaataaattc agcttgaatc acccaaatca |
| | 17401 | gatggcattc ttaaagccac tgagtggtaa aatcaggggt gtgcagccaa aacgtccatt |
| | 17461 | ttgactcatt atgatttcca tgtcacaaga ctagaaagtc actttctcct cagcagaaga |
| | 17521 | gaaggtagaa cattttaacc tttttttgga gtgtcaaggg aattttgttt acactgtaaa |
| | 17581 | gtcagtgaaa atattgaagc ttttcatttg tggaaaatat taaatatgta aaattgaaat |
| | 17641 | tttaaaattt attcctgggt agttttgttt ttccagtagt catgcatgga tgtgagagtt |
| | 17701 | ggactataaa gaaagctgag cgctgaagaa ttaatgctrt tgaactgtgg cactggagaa |
| | 17761 | gactcttgag agtcccttgg tctgcaagga gatcaaacca gtccatccta aaggaaatca |
| | 17821 | gtcctgaata ttcactggaa ggactgatgc tgaagctgaa actccaatac tttggccacc |
| | 17881 | tgatgtgaag aactgactca tatgaaaaga ctcagatgct gggaaagatt gaaggtggga |
| | 17941 | ggagaagggg acgacagagg atgagatggc tgaatggcat caccgactcg atggacatga |
| | 18001 | gtctgaataa gctctgggag ttgttgatgg acagggaggc cctggagtgc tgcagtccat |
| | 18061 | gggattgcaa agagttggac atgactgagt gactgaactg aactgagttt ggtaacagat |
| | 18121 | atgagaatta tataatttaa atctaaactc ttggtatttc tttctttggc ggttccaaaa |
| | 18181 | gagctgtccc ttctgttaac tatataaatc ctttttgaga attactaaat tgataatgtt |
| | 18241 | cacaagttat ccaatttctc attactctta gttgtcagta taagaaatcc catttgattt |
| | 18301 | atcatgttat agtatctgca actctaatag ttcagttctg acaaattttt attttattta |
| | 18361 | aaaatattgg catacagtaa aatttcaaac aatatacaat tctccctttc agtttaaaaa |
| | 18421 | acaaaacaaa acaaaagtaa tattagttaa aaaaatccgg gaagaatcca agcatttaaa |
| | 18481 | attgcatcac atttctatgc tagacaagct gatataaagt tataattaat aaaggattgg |
| | 18541 | actattaaac tctttacata tgaggtaaca tggctctcta gcaaaacatt taaaaatatg |
| | 18601 | ttgtgggtaa attattgttg tccttaaaga aataaaaaga cataagcgta agcaattggn |
| | 18661 | nnnnnnnnnn nnnnnnnnnn nnnnnnnnnn nnnnnnnnnn nnnnnnnnnn nnnnnnnnnn |
| | 18721 | nnnnnnnnnn nnnnnnnnnn nnnnnnnnnn nnnnnnnnna aaatggataa ggggggagga |
| | 18781 | catgggtagg ggagcgcgat ggaggaagta aggtggtcga gggagttggg gggggaataa |
| | 18841 | gtgggtaaaa gggaagcggg cggaaggagg gggaagcagg agagaggggt gggcgtcaga |
| | 18901 | tcggggggag gggtatgagg gagagggaat ggtagacggg gggtgggaag cataaaggaa |
| | 18961 | aagatagggg ggggaaaagt tagaagaaga atgaggggat aggcggaaag ggaagagaaa |
| | 19021 | tgggagaaga acagaaaaat agggggaggg ggggcgtaaa gagggggggg gagggcaggt |
| | 19081 | gtggagatga cagatacggg gaatgccccg gtataaaaga gtatatggcg tggggcgaga |
| | 19141 | aggctgtcat cctgtgggag gggggacgcg gagaaccctt cgggctatag ggaggattcg |
| | 19201 | gggggatcgt tcgggaaggc agtcagcaca gcacccacca agggtgcagg gatggatctg |
| | 19261 | gggtcccaaa gaagaggccc aatcccgcgt cttggcagca aggagccctg gagactggga |
| | 19321 | agtgtccagg acactgaccc aggggttcga ggaacccaga agtgtgtctg tgaagatgtg |
| | 19381 | ttttgtgggg ggacaggtcc agagctttga gcagaaaagc ggccatggcc tgtggagggc |
| | 19441 | caaccacgct gatctttttt aaaaggtttt tgttttgatg tggaccattt ttaaagtctt |
| | 19501 | cattgaattt gctacaatat tgtttctggt ttatgctctg gtttcttcgg ctgcaaggtt |
| | 19561 | tgtgtgatcg tatctcctca accaggactg aacccacagc ccctgcactg gaaggcgaag |
| | 19621 | tcttaaccca gatcgccagg aacgtccctc ccctcactga tctaatccaa gaccctcatt |
| | 19681 | aaggaaaaac cgagattcaa agctccccca ggaggactcg gtggggagga gagagccaag |
| | 19741 | cactcagcac tcagtccagc acggcgccct ccctgtccag ggcgagggct cggccgaagg |
| | 19801 | accaccggag accctgtcgg attcaccagt aggattgtga ggaatttcaa cttacttttt |
| | 19861 | aaatctgtct ctcaaggctg ttacaagcgg actttaccag taacttaaaa gttgaaaggg |
| | 19921 | acttcccagg cggcacttgc ggtgaagaac ccgccggctg gttttaggag acataagaga |
| | 19981 | tgtgggttag atccctggtt caggaggatt cccctggaga aggaaatggc aacccactcc |
| | 20041 | agtattcttg cctggaaagc ctcacggaca gaggaggctg gcgggctaca gtccacgggg |
| | 20101 | tcgcacacga ctgaatcgac ttagcttcaa gttgagacag gaagaggcag tgactggtgg |
| | 20161 | caaaacaccg cacccatgct cccaggggac ctgcagcgct ctggttcatg agctgtgcta |
| | 20221 | acaaaaatca acccaacgag aggcccagac agagggaagc tgagttcatc aaacacgggc |
| | 20281 | atgatgtgga ggagataatc caggaaggga cctgccaagc ccatgacaga ccggtgtcct |
| | 20341 | gtctgagggc cgtcctggca gagcagtgca gggccctccg agaccgcccg agctccagac |
| | 20401 | ccggctgggg gctacagggt ggggctgagc tgcaaggact ctgctgtgag ccccacgtca |
| | 20461 | gggaggatca ccttgtttgt tttctgagtt tctcttaaaa tagcctttat gggtcctggt |
| | 20521 | ctttggtttt aaaataacaa ctgttctccg taaacaacgt gaaaaaaaac aaacaggagg |
| | 20581 | aaaacaacgc agcccgggca tttcacccgg aagagccgcc tctaacactt tgacgggttg |
| | 20641 | ccttctattt taaccctgtt ttcattgtaa actgtaaaaa ccacatcata aataaattaa |
| | 20701 | aggtctctgt gaagtttaaa aagtaagcat ggcggtggcg atggctgtgc cacaccgtga |
| | 20761 | acgctcgttt caaaacggta aattctaggg accccctggt ggtccagtgg gtgagatttt |
| | 20821 | gcttccattg caggagccgt gggtttgatc cctggttggg gaactaagat cccacatgct |
| | 20881 | gtatggagtg gccaaaaaga attttttgta aatggtgagt tttaggtgac gtgaatttcc |
| | 20941 | cattgatgca cttcacaggc tcagatgcag ccaggccctc aggaagcccg agtccaccgg |
| | 21001 | tcctttactt ttccttagag ttttatggct tctgtttctg cccttaaacc caccatgttt |
| | 21061 | caacctcatc tgattttgga ctttataata aagttaggct gtgtttcagg aaactttgct |
| | 21121 | cagtattctg taataatcta aatggaaaga atttgaaaaa agagcagaca cttgtacatg |
| | 21181 | cataactgaa tcactttggt gtacacctga aactcgagtg cagccgctca gtcgtgtccg |
| | 21241 | accctgcgac cccacggact gcagcacgcg ggcttccctg cccatcacca actcccggag |
| | 21301 | ttcactcaaa cacatgtccg tcgactcggt gatgccgtcc aaccgtctca tcctctgtcg |
| | 21361 | tccccttctc ctcccgcctt caatcttttc cagcatcagg gtcttttcaa atgagtcagt |
| | 21421 | tcttcacacc aggtggccag agtattggag tttcagcttc agcatcagcc cttccaacga |
| | 21481 | ccccccatac ctgaagctaa cacagtgcta atccactgtg ctgcaacatg aaagaaaaac |
| | 21541 | acatttttta agtttaggct gtgtgtgtct tccttctctc aacactgcgt ctgaccccac |
| | 21601 | ccacactgcc cagcactgca ttccccgtgg acaggaggcc ccctgcccca cagctgcgtg |
| | 21661 | ccggccggtc actgccgagc agacctgccc gcccagagtg gggcccctgg cactggggac |
| | 21721 | aaggcagggg cctctccagg gccggtcact gtccactgtt cctactggtt ttgttttcaa |
| | 21781 | aagtggaggc agcgtaatat ttccctgatt ataaaaagaa gtacacaggt tctccacaaa |
| | 21841 | taaaacaggg gaaaagtata aagaatggaa gttcccagca cagcctggag atcacgccgg |
| | 21901 | gtgcacctgg ggtgtccttc caggctggac ctcacatttc acgcagacat cagaaggctg |
| | 21961 | cgagatctac ccagaaggct gggtagatgg gggataggtc agtgacaaac agtagacaga |
| | 22021 | gagatataca gacagatgat ggatagacag acgctaagac accgagcgag gggacagacg |
| | 22081 | gatggaagac accatccttt gtcactgacc acacacccac atgggtgtgg tgagccggct |
| | 22141 | gtcatacttg tgaacctgct gctctcacaa caccagctgg gtccctccag ccccagcgtc |
| | 22201 | ccacacagca gactcccggc tccatcccca ggcaggaatc ccaccaccaa ctggggtgga |
| | 22261 | ccctccccgc aggaaggtcg tgctgtctaa ggccttgaga gcaagttaca gacctacttc |
| | 22321 | tgggaagaca gcgcacaacc gcctaccccg cagagcccag gaggacccct gagtcctagg |
| | 22381 | gaagggacca cgcggcctgg acggggagcg gccccaggac gctgccccca acctgtccca |
| | 22441 | cctcactcct gctctgctct gaggcggggc gcagagaggg gccctgaggc ctcttcccag |
| | 22501 | ttcttgggag cacccactgg gcctgaacca ggccagaagc cccctcctca aggtgtcccc |
| | 22561 | agaccactcc cctccacctc cggttgctct gtctcctggc agcagggagc cccagtgaga |
| | 22621 | agagacagct ccaggctgtg atcttggccc ctggctgctc tggcagtgtg gggggtgggg |
| | 22681 | gtcgctggga ggccatgagt gctgggggtc ggggctgtga aagcacctcg aggtcagtgg |
| | 22741 | gctgttggtc gggctctgcg aggtccgcac gggtagagct gtgccaggac acaggaggcc |
| | 22801 | tggtcagtgg tcccaagagt cagggccaaa ggaaggggtt cgggcccctc tggttcctca |
| | 22861 | gcttctgagg ccggggaccc cagtctggcc ttggtagggg ggcgattgga gggtacaacg |
| | 22921 | atccaaaaga aaacacacat ctacgaggga agagtcctga ggaggagaga gctacacaga |
| | 22981 | gggtctgcac actgcggaca ctgcttggag tctgagagct cgagtgcggg gcacagtgag |
| | 23041 | cgaagggagg acggaacctc caaggacacc ggacgccgat ggccagagac acacgcacgt |
| | 23101 | cccatgaggg ccggctgctc agacgcaggg gagctcctca ttaaggcctc tcgctgaata |
| | 23161 | gtgaggagaa ctggccccgt gtgtggggaa acttagccca gaagaaacgc tgccctggcc |
| | 23221 | ccaaggatca nnnnnnnnnn nnnnnnnnnn nnnnnnnnnn nnnnnnnnnn nnnnnnnnnn |
| | 23281 | nnnnnnnnnn nnnnnnnnnn nnnnnnnnnn nnnnnnnnnn nnnnnnnnnn tgccctttgc |
| | 23341 | ctccagggag ggaggaagcg tggatcttgg gtttgccttg ggtttaaagg atccacccac |
| | 23401 | tcccttttta gccactccct gtgctggcaa tttcttaaga ctggaggtcg caaagagttg |
| | 23461 | gacacactga gcgagtgaac tgcactgagc ctaagaaaag tctttgaatt cctccaaaca |
| | 23521 | aaacacactt gtcttgggta ctttccttgg ttttgttaca aatgtctggt ccctctgttc |
| | 23581 | tcctggccag ctcctgggtg tcattttgac ctgacgaagt caaagggagc ctggaccctc |
| | 23641 | aaaatctgta ggacccagca cccctccatt acacctctgt tcccccgcga acgggcacgt |
| | 23701 | gtttcgccgt ctggcgtaat gtgtaagcga cggtgtgata ctcgggagtc ttactctgtt |
| | 23761 | tctttttctt ctggggtgac accaccatcc gcacgactct gtctgaatgt gaacatttgg |
| | 23821 | gtgatttgat gtggcccaga ctcccccaac gaatgtacct tcaggttggt tttcttcttt |
| | 23881 | tatattttgc ttttgtgaat agacacagga tcccatcagt tgtatgtagt gagaaagtaa |
| | 23941 | aaacccactc agccttagct ggatggagat ctagtagtaa gatagcacgt tagccggaaa |
| | 24001 | tggaaatttc agccagaatc tgaaaagcgt gtcctggaag gagaagaggg actcaggccc |
| | 24061 | gagcacactg ctccacgctg gagcctcagg ctctgacagc tgtacctgcc ggggtcttca |
| | 24121 | tgggacaggc catgcaggcc acgatcccgt tgagaagttt cttgcctttc catcacattg |
| | 24181 | gcaattgcac gctttgctct tgcttctaca tggagtttta cttttatccc agacagtttg |
| | 24241 | gtttcttctc tgattttcgc caattgtaca gatcgttaca gtatttctta accacataga |
| | 24301 | attcggcagg gggggtgggg ggacagggta gggtggggtg agagtgaggg gagggggctg |
| | 24361 | caccgagcag catctggggt cgtagctccc tgacggggat agacctcgtg cccctgcagt |
| | 24421 | gacagcacag agtcctcctc tctgaactgc cagggacgct cctgcaattg acttaatgaa |
| | 24481 | aggcatctaa ttaggaattt tggggtgaca ttttacattt aagtgtgtga gcagtgatta |
| | 24541 | tagttcatat cattttatag tttcgtgatt ttactagctt aaagggtttt tggggtttct |
| | 24601 | ttttgtttta aaagctaaaa tctgtttttt aattccatgg aatacaaaaa aaaaaagtct |
| | 24661 | gtagaatatt ttaaagagtg aaggctttgt tcggaatgtg agcgctttgc tccactgaac |
| | 24721 | cgaacggtaa taacatttgt agaagagacg cagagtgaaa ggtacctctt tttattgagt |
| | 24781 | gacatgacag cacccatcgc gtgagttatt ggctggagtt tagagacagg ccatgttggg |
| | 24841 | ctaaactcct tattgctgtt ctcagccttt gagtaataat cagaagcttt ctctgaagag |
| | 24901 | agtggggtca gctgtcagac tcctaggtgt ctacctgcag cagggctggg attaaatgca |
| | 24961 | gcagccagta gatacgggat ggggcaagag gtcaccttgt ccctttgttg ctgctgggag |
| | 25021 | agaggcttgt cctggtgcca gtggggccaa agctgtgact ttgtgaccac aggatgtctc |
| | 25081 | tgaccctgcc ttgggttccc tgagggtgga gggacagcag ggtctccccg gttccttggc |
| | 25141 | cggagaagga ccccccaccc cttgctctct gacatccccc caggacttgc cccggagtag |
| | 25201 | gttcttcagg atgggcatcc gggccccacc ctgactcctg gagctggccg gctagagctt |
| | 25261 | gctgcagaat gaggccttgg ccattgcggc cctgaaggag ctgcccgtca agctcttccc |
| | 25321 | gaggctgttt acggcggcct ttgccaggag gcacacccat gccgtgaagg cgatggtgca |
| | 25381 | ggcctggccc ttcccctacc tcccgatggg ggccctgatg aaggactacc agcctcatct |
| | 25441 | ggagaccttc caggctgtac ttgatggcct ggacctcctg cttgctgagg aggtccgccg |
| | 25501 | taggtaaggt cgacctggca gactggtggg gcctggggtg tgagcaagat gcagccaggc |
| | 25561 | caggaagatg aggggtcacc tgggaacagg cgttgggtgt acaggactgg ttgaggctca |
| | 25621 | gaggggacaa aaggcacgtg ggcctccccc ccagtgtccc ttaaagtggg aaccaagggg |
| | 25681 | gccccggaag ccggaggagc tgtggtgtgt ggagtgcaga gccctcgcgg ggtcctgatg |
| | 25741 | cccgtcggac tctgcacagc tcagcgtgtg ccccgcggcc cggtaggcgg tggaagctgc |
| | 25801 | aggtgctgga cttgcgccgg aacgcccacc agggacttct ggaccttgtg gtccggcatc |
| | 25861 | aaggccagcg tgtgctcact gctggagccc gagtcagccc agcccatgca gaagaggagc |
| | 25921 | agggtagagg gttccagggg tgggggctga agcctgtgcc gggccctttg gaggtgctgg |
| | 25981 | tcgacctgtg cctcaaggag gacacgctgg acgagaccct ctgctacctg ctgaagaagg |
| | 26041 | ccaagcagag gaggagcctg ctgcacctgc gctgccagaa gctgaggatc ttcgccatgc |
| | 26101 | ccatgcagag catcaggagg atcctgaggc tggtgcagct ggactccatc caggacctgg |
| | 26161 | aggtgaactg cacctggaag ctggctgggc cggatgggca acctgcgcgg ctgctgctgt |
| | 26221 | cgtgcatgcg cctgttgccg cgcaccgccc ccgaccggga ggagcactgc gttggccagc |
| | 26281 | tcaccgccca gttcctgagc ctgccccacc tgcaggagct ctacctggac tccatctcct |
| | 26341 | tcctcaaggg cccgctgcac caggtgctca ggtgaggcgt ggcgccagct ccaaagacca |
| | 26401 | gagcaggcct ctcttgtttc gtgcccgctg gggacattgc cagggtgccc ggccactcgg |
| | 26461 | aagtcctcac gatgccaccg ctctgaccct gggcatcttg tcaggtcact tccctggtta |
| | 26521 | gggtcagagg cgtggcctag gttaaatgct gtcaaagggg actcctttct gggagtccgc |
| | 26581 | atagtggggg cttggtgtga tgcccttggg aattctttcc gagagagtga tgtcttagct |
| | 26641 | gagataatga cagataacta agcgagaagg acggtccatc aggtgtgagg tttgaagtcc |
| | 26701 | aaagctctgt ctctccctcc cacctgcccc ttctgtcctg agctgtttta ggctccaggt |
| | 26761 | gagctgtggg aagtgggtga ttctggagat gacaagaagg gatcaggagg ggaaaattgt |
| | 26821 | ggctcctaag cagtccagag aagagaaaaa gtcaaataag cattattgtt aaagtggctc |
| | 26881 | cagtctcttt aagtccaaat tataattata attttcctct aagacttctg aatacatagg |
| | 26941 | aaatcctcag taacaggtta ttgctctgcc ttgaacacag tgataaaagc tgggaggatg |
| | 27001 | cagcctaatc tgtctgtgtg aatgagttgt attgattccc tttttggcag ctgcaaactc |
| | 27061 | caagcattag gaataaatat gttcactgag aaccccgaag aaagaaagaa agaaaaaaaa |
| | 27121 | aaagaattgt aggtgttgat ggacggtttg tggcccctga atatctgggg gatgttcacc |
| | 27181 | cagggatcac gtgtaactgc tgggaccccc agccccatgt ccactgcatc cagcctgctg |
| | 27241 | ttgaattccg cggatcnnnn nnnnnnnnnn nnnnnnnnnn nnnnnnnnnn nnnnnnnnnn |
| | 27301 | nnnnnnnnnn nnnnnnnnnn nnnnnnnnnn nnnnnnnnnn nnnnnnnnnn nnnnnncaat |
| | 27361 | tcgagctcgg taccccaaag gtccgtctag tcaaggctat ggtttttcca gtggtcatgt |
| | 27421 | atggatgtga gagttggact gtgaagaaag ctgagtgcca aagaattatt cttttgtact |
| | 27481 | gggtgttgga gaagactctt gagagtccct tgaactgcaa ggagatccaa ccagtccgtt |
| | 27541 | ctaaaggaga tcagtcctga atgttcattg gaaggactga tgctgaagct gaaactccaa |
| | 27601 | tactttggcc acctgacgtg aagagttgac tcattggaaa agaccatgat gctgagagga |
| | 27661 | attgggggca ggaggagaag gggacgacag aggatgagat ggctggatgg catcaccaac |
| | 27721 | tcgatgngac atgagtttgg ttaaactcca ggagttggtg atggacttgg aggcctggtg |
| | 27781 | tgctgggatt catggggtcg cagagtcgga catgactgag cgactgaact gaactgaact |
| | 27841 | gagctgaaga gctcacctgt accagagctc ctcaggtcct cctgcaggcc tggctgtaat |
| | 27901 | ggcccccagg tcaccgtcct gcctccttca tcccatcctt tcacgacagg ctgggagtgg |
| | 27961 | ggtgaggtga gttgtcttgt atctagaatt tctgcatgcg accctcagag tgcaatttag |
| | 28021 | ctccagagaa ctgagctcca agagttcatt ttttcctttt cttctttatg atactaccct |
| | 28081 | cttctgagca gagacctcat gtcagggaga aggggactct gccttcctca gccttttgtt |
| | 28141 | cctccaagac ccacacgggg agggtcgcct gcttcactga gccggaaggt tcaattgctc |
| | 28201 | atgtcctcca gaaacacccc cccccccaga gacccccaga aataagtgga acagcacctt |
| | 28261 | gtttcccaga caagtgggac acacgttatg aaccacctca gtgattaaaa tagtaacctc |
| | 28321 | tgtgtatgtg tatttactgg agaaggaaac ggcaacctac tccactattc ctgcctagaa |
| | 28381 | aattccatgg gagagaagcc aggcaggcta cagtccacgg ggtcacagag actgaacata |
| | 28441 | cacaagcaca tggaagtgta ttttgcagta tttttaaatt tgttcagttc aacatggagt |
| | 28501 | acaagaattc aaatcgtgaa gtcaattgac caagaaacca gaagaaatca ctgtgttgtg |
| | 28561 | atctctgtgg aggtaacatg ggtacctgtg ctctgaccct cacagcctct ggctctctct |
| | 28621 | ctacatgtac atacacatat atttccatgt atgtatgtat tcggaagatt tcacatacgt |
| | 28681 | ctcaccagtc cacagccccc gcgttccctg atgcccagaa catctgtgat agctgtgagt |
| | 28741 | attgtcacca gataagatct tccaggttcc tgcactcaca ttggttatca ggtctctctg |
| | 28801 | atccagcatt tctcagctaa gattccttgt gactcctggc tgcagaatct tctgcaaaag |
| | 28861 | tcccacagag aggagtgtga tcactgtaca caggagggcc gtggttctct agtgtgagaa |
| | 28921 | aagctaactc agcccgtcac agggacgtga atgtacctga gacagtaatc agttatgctg |
| | 28981 | agaaatcaca gctctgctag aggcagcaca tggggtagcc agcagggggc agcagagcac |
| | 29041 | ggccaggagc cgcaggtcag aggctgggct gcccaagcgg ggcttcaggg gaaccagccc |
| | 29101 | tgcgggtcca caggtgtcca gggagcagcg cttggcagga agtcaggacc ggacaggcca |
| | 29161 | tcccctcagg actagtgacc acctctgagg gtcacatcca cagtgaaccc cagagcacca |
| | 29221 | tgcctcagtc cacggccagg acgctgccag gctgaccgcc ccactgggga gtccagggga |
| | 29281 | gaccacaggc cggggggctt gggacagtga tcatgtggtc agacacagag aaggtgacag |
| | 29341 | tgacctcagt ccctgaggac aagtctgatg tgcagacgtg agaagccgag gaggaagctg |
| | 29401 | gggacagaca gggctgatgg tgtggtgacc ccgcctctca gtgaggggcc cccgggggtg |
| | 29461 | aatttgcata aacccaagcc ctcactgccc ccacaaagct ctgagaggga ataaaggggc |
| | 29521 | tcggagagcc cagcactgct gcgggctcag aggcagagct cggggcgcgt ccaccatggc |
| | 29581 | ctgggcccct ctcgtactgc ccctcctcac tctctgcgca ggtgcggccc cccagcctcg |
| | 29641 | gtccccaagt gaccaggcct caggctggcc tgtcagctca gcacaggggc tgctgcaggg |
| | 29701 | aatcggggcc gctgggagga gacgctcttc ccacactccc cttcctctcc tctcttctag |
| | 29761 | gtcacctggc ttcttctcag ctgactcagc cgcctgcggt gtccgtgtcc ttgggacaga |
| | 29821 | cggccagcat cacctgccag ggagacgact tagaaagcta ttatgctcac tggtaccagc |
| | 29881 | agaagccaag ccaggccccc tgtgctggtc atttatgagt ctagtgagag accctcaggg |
| | 29941 | atccctgacc ggttctctgg ctccagctca gggaacacgg ccaccctgac catcagcggg |
| | 30001 | gcccagactg aggacgaggc cgactattac tgtcagtcat atgacagcag cggtgatcct |
| | 30061 | cacagtgaca cagacagacg gggaagtgag acacaaacct tccagtcctg ctcacgctct |
| | 30121 | cctccagccc cgggaggact gtgggcacag cagggacagg cctggcccgg ttcccccgga |
| | 30181 | gctgagcccc caggcggccc cgcctcccgg ccctccaggc aggctctgca caggggcgtt |
| | 30241 | agcagtggac gatgggctgg caggccctgc tgtgtcgggg tctgggctgt ggagtgacct |
| | 30301 | ggagaacgga ggcctggatg aggactaaca gagggacaga gactcagtgc taatggcccc |
| | 30361 | tgggtgtcca tgtgatgctg gctggaccct cagcagccaa aatctcctgg attgacccca |
| | 30421 | gaacttccca gatccagatc cacgtggctt tagaaaggct taggaggtga acaagtgggg |
| | 30481 | tgagggctac catggtgacc tggaccagaa ctcctgagac ccatggcacc ccactccagt |
| | 30541 | actcttccct ggaaaatccc atggacggag gagcctggaa ggcttcagcc catggggtcg |
| | 30601 | ctaagagtca gacacgactg agcgacgtca ctttcccttt tcactttcat gcattggaga |
| | 30661 | aggaaatggc aacccagtcc agtgttcctg cctggaaaat cccagggaca ggggagcctg |
| | 30721 | gtgggctgcc atccatgggg ccacacagag tcagacacga ctgaagcaac ttagcagcag |
| | 30781 | cagcagcagc ccaataaaac tcagcttaag taatggcatc taaatggacc ctattgccaa |
| | 30841 | ataaggtcca ctcgcgtgca ctctgtttag gacttcagtt cctgattgtg gagggttccc |
| | 30901 | acaagacgtg tgtgtatatt ggtgttgccg gaaaacagtg tcaatgtgag catcccagac |
| | 30961 | tcatcaccct cctactccca ctattccatt gtctctgcag gtattaagca taaaggttaa |
| | 31021 | gggtcttatt agatggaaga ggagtgaata ctcgtctgtg cttaacacat accaagtacc |
| | 31081 | atcaaggtcc ttcctattta ttaacgtgtg ttttaatcag aaatatgcta tgtagaagca |
| | 31141 | tccggacgat agcccatgtt acagacgggg aagctgaggc atgaagttct cagcaccttg |
| | 31201 | tttcacgtca gacctgaaac ggggcagagc cggcagcaaa caaggttcct cttcccaagc |
| | 31261 | gcccgctctt cacccgcttc ctatggcttc tcactgtgct tcctaaacta agctctcccc |
| | 31321 | aaccctgtgg agacaggatt agagacttta ggagaaaaga ccaggaacat cccacacccg |
| | 31381 | acccgagtga gccactaaga caaggctttg taaggacaga accagcaggt gtcctcagcg |
| | 31441 | agccagggag agacctcgca ccaaaaacaa tattgtagca tcctgaccct ggacttctga |
| | 31501 | cctccagaaa tgtgaaaaag aaacgtgtgg ggtttaatca actcaccggt gttatttggt |
| | 31561 | tatgactgcc tgagttaaga aggagttggg aacacttgag tgtaggtgtt tatggaacat |
| | 31621 | aagtcttgtt tctctgaaat aaattcccaa gggtataatt cctaggttgt agggtaactg |
| | 31681 | ccacaaatct aggcagctta ttaaaaaaca aagatatcac tttgccagca aaggttcata |
| | 31741 | tagtcaaatt atggttttta tagtagtcat gtatggatgt aaaagttgga tcataaagaa |
| | 31801 | ggctgagcac cagagaattg atcccttcaa atcgtggtgc tggagaagac tcttgagagt |
| | 31861 | cccttggaca gcaaggagat ccaaccagtc aatcctaaag gaaatgaact gtgaatattc |
| | 31921 | actggaagga ctgatgctga agctgaagat ccaatacttt ggccacctga tgcgaagagt |
| | 31981 | tgactcattg gaaaagaccc tgatgctgga aagcttgagg gcaggaggag aagagggcgg |
| | 32041 | cagaggatga gacggttgga tggcatcact gactcaatgg acatgagttt gagccaactc |
| | 32101 | tgggagacag tgaaggatag ggaaggctgg cgtggtacag tgcatgcggt cacaaagagt |
| | 32161 | ctgacacatc ttagtgactc aacaacgaca gcaacacagg catcacacgc ttagtgtgat |
| | 32221 | aagcggcaga actgttttcc aggggtccgn nnnnnnnnnn nnnnnnnnnn nnnnnnnnnn |
| | 32281 | nnnnnnnnnn nnnnnnnnnn nnnnnnnnnn nnnnnnnnnn nnnnnnnnnn nnnnnnnnnn |
| | 32341 | nnnnnnnnng tacgattcga gctcggaccc tgacattgtg agtcacgtca tgagcagctg |
| | 32401 | ttttccggtc ttcagggatt gtggacgatt tctgtttggg tttgctcatg ataatttagt |
| | 32461 | tacagcttag gttctttctt tccaggccac gagcgacatg ttttcaggtg agatgacgtg |
| | 32521 | gtgggggatg ggcggccaag cccccactgg ggggggaggg attctgttgt gggcaggagt |
| | 32581 | tggcagcatc cctgaactga tgacctgcga tccaggtgac aagaaccggg ggatattatt |
| | 32641 | cctctgcctt ctcatgtcat gtcctcggtt cttcatgatg aaaacatatg acaatacagg |
| | 32701 | ggagttagat ttgggcgggc acaactctgg gtgggggacc cggtggcatt gtgcccagca |
| | 32761 | gggccatcaa gatgagggcg acctgggtgg tccccttctc ccctggggtc ttagttttcc |
| | 32821 | cctcatggaa atgggatcag gcagcagcca tggaacaccg cgaccgtggc ttctctcacc |
| | 32881 | tcctcgtctg tgattttggg tcgggatacc aggcatgaag acctggggcg gggggacatc |
| | 32941 | actcctctgc agcagggagg ccgcagagtc ctccgtccat gaggacttcg tccctgggct |
| | 33001 | gaccctgcgg actgctggag gctgaagctg gaggcacagg cgggctgcga ggccagggtc |
| | 33061 | ctgaggacga cagagccagt ggggctgcag ctctgagcag atggcccctc gccccgggcc |
| | 33121 | ctgagcttgt gtgtccagct gcaggttcgc tcaggtgagc cactacgtta tgggggaggc |
| | 33181 | gccctgggca gggatcgggg gtgctgactc ctccgagatt ccgaccttct gggagcactc |
| | 33241 | tggccacact ctaagcctgg caagagctgg gttcatcagt ctaactctcc tcctgaagtc |
| | 33301 | caatggactc tctccatgcg gcagtcactg gatggcctct ttatccccga tggtgtcctt |
| | 33361 | ttccgctgac ctggctctcc tgaccacctc ccagcccccc accatacagg aagatggcac |
| | 33421 | ctggtccctg cagagctaag tccacccctg gcctggcttc agatgcctac agtcctcctg |
| | 33481 | cgggaggccc cgctccccac taggccccaa gcctgccgtg tgagtctcag tctcacctgg |
| | 33541 | aaccctcctc atttctcccc agtcctcagc tcccaacccc agaggtatcc cctgcccctt |
| | 33601 | tcaaggccct tgtcccttcc tggggggatg gggtgtatgg gagggcaagc ctgatccccc |
| | 33661 | gagcctgtgc cgctgacaat gtccgtctct ggatcatcgc tcccctggct ctcagagctc |
| | 33721 | cctggtccct ggggatgggt tgcggtgatg acaagtggat ggactctcag gtcacacctg |
| | 33781 | tcccttccct aaggaactga cccttaaccc cgacactcgg ccagacccag aaagcacttc |
| | 33841 | agacatgtcg gctgataaat gagaaggtct ttattcagga gaaacaggaa cagggaggga |
| | 33901 | ggagaggccc ctggtgtgag gcgacctggg taggggctca ggggtccatg gagaggtggg |
| | 33961 | ggagggggtg tgggccagag ggcccccgag ggtgggggtc cagggcccta agaacacgct |
| | 34021 | gaggtcttca ctgtcttcgt cacggtgctc ccctcgtgcg tgacctcgca gctgtaactg |
| | 34081 | cctttcgatt tccagtcgct gcccgtcagg ctcagtagct gctggccgcg tatttgctgt |
| | 34141 | tgctctgttt ggaggcccgg gtggtctcca cgttgcgggt gatggtgctg ccgtctgcct |
| | 34201 | tccaggccac ggtcacgcta cccgggtaga agtcgctgat gagacacacc agggtggcct |
| | 34261 | tgttggcgct gagctcctcg gtggggggcg ggaacagggt gaccgagggt gcggacttgg |
| | 34321 | gctgacccgt gtggacagag gagagggtgt aagacgccgg ggaggttctg accttgtccc |
| | 34381 | cacggtagcc ctgtttgcct tctctgtgcc ctccgaccct tgccctcagc ccctgggcgg |
| | 34441 | cagacagccc ctcagaagcc attgcaatcc actctccaag tgaccagcca aacgtggcct |
| | 34501 | cagagtcccc ggctgcgacc agggctgctc tcctccgtcc tcctggcccc gggagtctgt |
| | 34561 | gtctgctctt ggcactgacc ccttgagccc tcagcccctg ccagacccct ccgtgacctt |
| | 34621 | ccgctcatgc agcccaggtg cctcctccgt gaacccgggt ccccccgccc acctgccagg |
| | 34681 | acggtcctga tgggagatgt ggggacaagc gtgctagggt catgtgcgga gccgggcccg |
| | 34741 | ggcctccctc tcctcgccca gcccagcctc agctctcctg gccaaagccc ggggctcctc |
| | 34801 | tgaggtcctg cctgtctacc gtccgccctg cctgagtgca gggcccctcg cctcacctgc |
| | 34861 | cttcagggga cggtgccccc acacagcacc tccaaagacc ccgattctgt gggagtcaga |
| | 34921 | gccctgttca tatctcctaa gtccaatgct cgcttcgagg ccagcggagg ccgaccctcg |
| | 34981 | gacaggtgtg acccctgggt cccaggggat caggtctccc agactgacga gtttctgccc |
| | 35041 | catgggaccc gctcctttct gaccgctgtc ctgagatcct ctggtcagct tgccccgtct |
| | 35101 | cagctgtgtc cacccggccc ctcagcccag agcgggcgag acccctctct ctctgccctc |
| | 35161 | cagggccttc cctcaggctg ccctctgtgt tcctggggcc tggtcatagc ccccgccgag |
| | 35221 | cccccaagct cctgtctggc ctcccggctg gggcatggag ctcacagcac agagcccggg |
| | 35281 | gcttggagat gcccctagtc agcaccagcc tctggcccgc accccagcgt ctgccctgca |
| | 35341 | agaggggaac aagtccctgc attcctggac caaacaccag ccccggcgcc ccgactggcc |
| | 35401 | ccattggacg gtcggccact ggatgctcct gctggttacc ccaagaccaa cccgcctccc |
| | 35461 | ctcccggccc cacggagaaa ggtggggatc ggcccttaag gccgggggga cagagaggaa |
| | 35521 | gctgccccca gagcaagaga agtgactttc ccgagagagc agagggtgag agaggctggg |
| | 35581 | gtagggtgag agccacttac ccaggacggt gacccaggtc ccgccgccta agacaaaata |
| | 35641 | cagagactaa gtctcggacc aaaacccgcc gggacagcgc ctggggcctg tcccccgggg |
| | 35701 | gggctgggcc gagcgggaac ctgctgggcg tgacgggcgc agggctgcag ccggtggggc |
| | 35761 | tgtgtcctcc gctgaggggt gttgtggagc cagccttcca gaggccaggg gaccttgtgt |
| | 35821 | cctggaggtg ccctgtgccc agccccctgg ccgaggcagc agccacacac gcccttgggg |
| | 35881 | tcacccagtg ccccctcact cggaggctgt cctggccacc actgacgcct tagcgctgag |
| | 35941 | ggagacgtgg agcgccgcgt ctgtgcgggg cggcagagga gtaccggcct ggcttggacc |
| | 36001 | tgcccagccg ctcctggcct cactgtaagg cctctgggtg ttccttcccc acagtcctca |
| | 36061 | cagtccagcc aggcagcttc cttcctgggg ctgtggacac cgggctattc ctcaggcccc |
| | 36121 | aagtggggaa ccctgccctt tttctccacc cacggagatg cagttcagtt tgttctcttc |
| | 36181 | aatgaacatt ctctgctgtc agatcactgt ctttctgtac atctgtttgt ccatccatcg |
| | 36241 | atccaacatc catccatcca tccatcaccc agccatccat ctgtcatcca acatccatcc |
| | 36301 | ttccatccat tgtccatcca tctgtccatc ttgcatctgt ctgtccaaca gtggccatca |
| | 36361 | agcacccgtc tgccaagccc tgtgtcacac gctgggactt ggtgggggga gccctcgccc |
| | 36421 | tcccaccctc ccatctctcc tgaaacttct ggggtcaagt ctaacaaggt cccatcccgt |
| | 36481 | ctagtctgag gtccccccgc agcctcctct tccactctct ctgcttctga cccacactgt |
| | 36541 | gcactcggac gaccacccag ggcccttgca tccctgtttc cttcctgacc tctttttttt |
| | 36601 | ggctctggat ttatacacat tctgcctcct ggaggcgtct cagcttgagt gtcccacaga |
| | 36661 | cgcctcagac tcagcatctt ccatcgaaac tgctcccagg tccttgcaga cctggtcccc |
| | 36721 | cacattgttc tcaattcggt agatttctcc acaagccaga ggcctggact catcccataa |
| | 36781 | tgcctgcccc tcattgagtc agcctctgtg tcctaccata accaaacatc cccttaaaaa |
| | 36841 | tctcagaaga acaaaaaaag cacccagatg gcactgtcag agtttatgat gacaagaatc |
| | 36901 | ctcagttcag ttcagtcact cagtcgtgtc cgactctttg cgaccccatg aatcgcagca |
| | 36961 | cgccaggcct ccctgtccat caccaactcc cggagttcac tcagactcac gtccattgag |
| | 37021 | tcagtgatgc catccagcca tctcatcctc tctcgtcccc ttctcctcct gcccccaatc |
| | 37081 | cctcccagca tcagagtttt ttccaatgag tcaactcttc gcgtgaggtg accaaagtac |
| | 37141 | tggagtttca gcttcagcat cattccttcc aaagaaatcc cagggctgat ctccttcaga |
| | 37201 | atggactggt tggatctcct tacagtccaa gggactctca agagtcttct ccaacaccac |
| | 37261 | agttcaaaag cctcaattct ttggcgctca gccttcttca cagtccaact ctcacatcca |
| | 37321 | tacatgacca caggaaaaac cataaccttg actagatgga cctttgttgg caaagtaatg |
| | 37381 | tctctgcttt ttaatatgcn atctaggttg ctcataactt tccttccaag aagtaagtgt |
| | 37441 | cttttaattt catggctgca atcaacatct gcagtgattt tggagcccca aaaaataaag |
| | 37501 | tctgccactg tttccactgt ttccccatct atttcccatg aagtgatggg accagatgcc |
| | 37561 | atgatctttg ttttctgaat gttgagcttt aagccaactt ttcactctcc actttcactt |
| | 37621 | tcatcaagag gctttttagt tcctcttcac tttctgccat aagggtggtg tcatctgcat |
| | 37681 | atctgaggtt attgatattt ctcctggcaa tcttgattcc agtttgtgtt tcttccagtc |
| | 37741 | cagtgtttct catgatgtac tctgcatata agttaaataa gcagggtgat aatatacagc |
| | 37801 | cttgacgtac tccttttcct alttggaacc agtctgttgt tccatgtcca gttctaactg |
| | 37861 | ttgcttcctg acctgcatac agatttctca agaggcaggt caggtggtct ggtattccca |
| | 37921 | tctctttcag aattttccac agttgattgt gatccacaca gtcaaaggct ttggcatagt |
| | 37981 | caataaagca gaaatagatg tttttctgaa actctcttgc tttttccatg atccagcaga |
| | 38041 | tgttggcaat ttgatctctg gttcctctgc cttttctaaa accagcttga acatcaggaa |
| | 38101 | gttcacggtt catgtattgc tgaagcctgg cttggagaat tttgagcatt cctttgctag |
| | 38161 | cgtgtgagat gagtgcaatt gtgcggcagt ttgagcattc tttggcattg cctttctttg |
| | 38221 | ggattggaat gaaaactgac ctgttccagg cctgtggcca ctgttgagtt ttcccaattt |
| | 38281 | gctggcatat tgagtgcagc actttcacag catcatcttt caggatttga aatcgctcca |
| | 38341 | ctggaattcc atcacctcca ctagctttgt ttgtagtgat gctctctaag gcccacttga |
| | 38401 | cttcacattc caggatgtct ggctctagat gagtgatcac accatcgtga ttatctgggt |
| | 38461 | cgtgaagatc ttttttgtac agttcttctg tgtattcttg ccacctcttc ttaatatctt |
| | 38521 | ctgcttctgt taggcccata ccgtttctgt cctcgcctat cgagccctcg cctccctacg |
| | 38581 | tagagactct aagcaggaag gtgacccgtg ctgcactggg tccagcatgc ttttaattca |
| | 38641 | gcagtggaac ttctgggtca tgattgtgtt taagggatgc gcatacgatt tttgaagcaa |
| | 38701 | aatttaacag gacagcagtg taaagtcagt acttatttct gattaaagaa agcaaatatc |
| | 38761 | cagcctgtta ctaagttaat taactaaaga aacatcttca acttaataaa cagtatctcc |
| | 38821 | tgaaacttac agcatgcttc acatttaaag gcaaaaccat tttagaggcc agggttccca |
| | 38881 | cgcttacgtt tattatttaa tatatgctac agattcaagc ccatgacaca aaatgggggg |
| | 38941 | aagagtgtga gtgttaggaa aaatgagata aaattggttt ttgcaggtga tgggctagtt |
| | 39001 | tactttaaaa aaaaaaacaa aacaagctca agatgaactg aaggactatt agaactggta |
| | 39061 | caagagttaa cctgtgatcg aatacaagca ggctgggcaa aactcagcag gttttcttct |
| | 39121 | atacaggcag taatgattga gaatacgaaa cggcggaagc gcttacaacc tcgataacag |
| | 39181 | ttctattaaa agccctagga atgaacttaa cacggnnnnn nnnnnnnnnn nnnnnnnnnn |
| | 39241 | nnnnnnnnnn nnnnnnnnnn nnnnnnnnnn nnnnnnnnnn nnnnnnnnnn nnnnnnnnnn |
| | 39301 | nnnnnnnnnn nnnnngctcc ccccaccctc ccctcctccc cccccaccac cagtgcccca |
| | 39361 | ggtctcgtgc ccagagagct gaagatgcca gcaggcccgc tgcctgcctc gctcgcgtgg |
| | 39421 | cccgggctcg ctgccggtct gcctgcccag cacacagatg cagccccagc tctcgctgcc |
| | 39481 | acccgcctcc cccaggcagg actctcccac aacaccaagg gcgtctctgg gttcaggatg |
| | 39541 | gccctcgttg aggtgtaaag tgcttcccgg ggctgagacg aatgggccgg agatccaaac |
| | 39601 | gaggccaagg ccgccacggc gcctggcgca gggcacccat ggtgcagagc ggcccagctc |
| | 39661 | cctccctccc tccctccctc cctgcttctt tatgctcccg gctatgtcta tttttactct |
| | 39721 | gcaatttaga aatgataccg aaggacaaac accgttcccc ctgtgtgtct gctctaaacc |
| | 39781 | ctttatctac ttatctatta gcgtgtccaa gttttgctgc taagtgaatg aaggaacact |
| | 39841 | acccacaagc agcaacgtcc ccacgaccct cgcctgttca actgggaatg taaatgtgct |
| | 39901 | ttcaaaggac ctaagtttct atgttcaaaa ccgttgtgtg tttcttttgg gagtgaacct |
| | 39961 | aggccactcg ttgttctgcc tttcaaagca ttcttaacaa ctctccagaa cccagggctt |
| | 40021 | ggcttacgtt tccagaaatt ccaaagacag acacttggaa acctgatgaa gaaggcctgt |
| | 40081 | gagcacagca ggggccgggg tacctgaggt aggtgggggg ctcggtgctg atggacacgg |
| | 40141 | ccttgtactt ctcatcgttg ccgtccagga tctcctccac ctcggaggct ttcagcaggg |
| | 40201 | tcacgctggt ggccagggtc gtgtatccat gatctgcaac cagagacggg gctgcggtca |
| | 40261 | gcccgcgggc gggcagcagg caggagcagc caggagacgc agcacaccga ggtcctcaca |
| | 40321 | tgcaggaggt gggggaagcg gctgtggacc tcacgactgc ccgatgtggg cctcttccaa |
| | 40381 | agggccggcc tggaccctgg ctttctccag aggccctgct gggccgtccg cacaggctcc |
| | 40441 | agccacaggg cctcttggga caggagggct ccagagtgag ccggccggcg ggaagaggtc |
| | 40501 | tgacaccgct gcagtccaca acacgaagcg aggtggagat gggatgaggg atgagaaaca |
| | 40561 | cttttctttt aaaacaagag cccagagagt tggaaagagc tgctgcacac gcaacatgaa |
| | 40621 | ctcctggccc cggtgccagc ggcgctggga gcccgagttc tcggcaatcc gaccacagct |
| | 40681 | tgcctaggga gccgggtgga gacggagggt taggggaagg cggctcccca gggagcgcga |
| | 40741 | ggcccggggt cgccaaggct cgccaggggc aagcgcagct aggggcgcag ggttagtgac |
| | 40801 | cggcactgca cccggcgcag gagggccagg gaggggctga aaggtcacag cagtgtgtgg |
| | 40861 | acaagaggct ccggctcctg cgttaaaaga acgcggtgga cagaccacga cagcgccacg |
| | 40921 | gacacactca taccggacgg actgcggagt gcacgcgcgc gcacacacac acacacacca |
| | 40981 | cacacacaca cacacggccc gggacacact cataccggac ggactgcgga gtgcacgcgc |
| | 41041 | acacacacac ccaccacaca cacacccacc acacacacac ccaccacaca cacacacaca |
| | 41101 | cacacacacc cccacacaca cccacacaca cccacacaca cccacacaca cacacccaca |
| | 41161 | cacacacaca cacacacaca cacacacacg gcccggtggc cccaggcgca cacagcacgg |
| | 41221 | agcaaacatg cacagagcac agagcgagcg ctagcggacc ggctgccaga ccaggcgcca |
| | 41281 | cgcgatggat tgggggcggg gacggggagg ggcgggagca aacggnnnnn nnnnnnnnnn |
| | 41341 | nnnnnnnnnn nnnnnnnnnn nnnnnnnnnn nnnnnnnnnn nnnnnnnnnn nnnnnnnnnn |
| | 41401 | nnnnnnnnnn nnnnnnnnnn nnnnngtatt aaagaagccg ggagcgagaa tatgacggca |
| | 41461 | agaggatgta ggtgggggcg gggcaagagt aaagagagcg gacggtagag gggatgcgat |
| | 41521 | tgtgatgcgg aagcgagacg aggagtgatg ccgtattaga ttgatagcaa gaggaacagt |
| | 41581 | aggagggggg ggggagagga gggggaggtg gggggtggtg ggtgggaagg gaactttaaa |
| | 41641 | aaaaagaggg gagagttgga ggggggaata aacgggcggt aaaaaagaac aatttgaaat |
| | 41701 | taccagggtg gggcggccag gggggtgatt cattcttgga gggggcaaca tatggggggt |
| | 41761 | ggctgtcgcg gattaggaga aaataaatat caggggtgat taagtgtttg gcgttgggga |
| | 41821 | ataatgaagt aagaatcaaa tatgaatcgc gttggcatcg ttagccatcg ggggaaacat |
| | 41881 | ttcccatgca aggaacaagg atgtgagaat gcgtccgtct gaaccaccgt cccggggtcc |
| | 41941 | cagtaggact cgccgagctg atagttgccg gagcaacagt taagggagca gaagctgcta |
| | 42001 | caaaaccacc acctgccaaa gtagggtctc caattacgga gtgcgcctcc tgggtgtcgg |
| | 42061 | tccaaacctt tggaaaggac ctggaaataa gtgctaccca ccagatatta atataaaccc |
| | 42121 | acctggccag gagaggcagg cgctgctggc acaggaagtg tccccagact cagtcatcaa |
| | 42181 | ggtaaataat attttgggac ctccctggaa atccagtggt taggactctg cggttcaatc |
| | 42241 | cctggtcggg gaactaagat cccacaagtc acaagacatg gccaaattta aaaaagaaaa |
| | 42301 | aaagagagag aaatatttag tgcaataggt tttagaattg aaattaagct cctgcccacc |
| | 42361 | cccacccccc aatctggatg aataaagcat tgaaatagta agtgaagtca ggctctgaca |
| | 42421 | tgcactgatg tgactcacct taagcaaccc ccaccctagg actggtcggg gttccaggag |
| | 42481 | tttcaggggt gccaggaaga tggagtccag cccctgccct ctccccccac cacgtcctcc |
| | 42541 | actggagccg cctaccccac ctcccacccc tccgcaccct gctacccccc acccctgccc |
| | 42601 | ccaggtctcc cctgtcctgt gtctgagctc cacactttct gggcagtgtc tccctctaca |
| | 42661 | gctggtttct gctgcccgct accgggcccg tcccctctgt tcagttcagt tcagtcgctc |
| | 42721 | agtcatgtct gactctttgt gaccccatgg actgcagcac accaggcctc cctggccatc |
| | 42781 | accaaccccc agaacttact caaactcatg tccatcgagc cagtgatgcc atccaaccat |
| | 42841 | ctcatcctct gtcgacccct tctcctggcc tcaatctttc ccagcatcag ggtcttttcc |
| | 42901 | aatgagtcag ttctttgcat caggtagcca aagtattgga gtttcagctt cagcatcatt |
| | 42961 | tcttccaatg aatattcagg actcatttcc tttgggatga actggttgga tctccttgca |
| | 43021 | gtccaaggga ctctcaagag tcttctccaa caccacagtt caaaagcatc aattcttcag |
| | 43081 | tgctcagctc tctttatagt ccaactctca catccatacg tgaccactgg aaaaaccata |
| | 43141 | gcctcgacta gatggaactt tgtgggcaaa gtaatgtctc tgcttttgaa tatgctgtct |
| | 43201 | aggttggtca taacttttct tccaaggagc aagcgtcttt taatttcatg gctgcagtca |
| | 43261 | ccatctgcag tgatttttgg agcccaagaa aataaagtct gtcactgttt ccactgtttc |
| | 43321 | cccgtctatt taacggaggg aaatttccca gagcccccag gttccaggct gggccccacc |
| | 43381 | ccactcccat gtcccagaga gcctggtcct cccaggctcc cggctggcgc tggtaagtcc |
| | 43441 | caggatatag tctttacatc aagttgctgt gtgtcttagg aaagaaactc tccctctctg |
| | 43501 | tgcctctgtt ccctcatccg cagaagtgac tgccaggtcg gggagtctgt gacgtctcca |
| | 43561 | gaagccggag gattttctcc ccatttgctg aaagagagct cggggtgggg gaagcttctg |
| | 43621 | cacccctagg atcaccagag gagccagggt cttcagggtt cccggggacc cctcagtggg |
| | 43681 | ggctcaggaa ccacagagcc agaccctgat tccaaaaacc tggtcacacc tccagatgac |
| | 43741 | cctttgtccc ttggctccgc ctcaaatgct ccaagcccca acagtgaagc gcttaagaga |
| | 43801 | aggatccacc aggcttgagt ttggggagga gggaagtggg gagctggggg agggcctggg |
| | 43861 | cctgggagac aggaatccac catggcttca ggcagggtct ctggggcctg cggggtggag |
| | 43921 | agcgggcagg agcagacaga ggtgactgga cacgacacac ccctccactc caagggaggt |
| | 43981 | gggcaggggc ggggcacaga ggaacaagag accctgagaa ggggtccacc gagcagactg |
| | 44041 | ctggacccag acatctctga gccagctgga atccagctct aagccatgct cagcccaggc |
| | 44101 | agggtatagg gcaggactga gtggagtggc cagagctgca gctgcatggg ctgggaaggc |
| | 44161 | cctgcccgtc ccctgagggt cccccagggt ctagccagac tccaatttcc gaccgcagca |
| | 44221 | cacacaggag gaagtggtcg gggtggagtt ggcccagagg tctgggcagg tgcagggtgg |
| | 44281 | gggaaggggg gcagctggag tcacccgctg aattcaggga cagtcccttt ttctccctga |
| | 44341 | aacctggggc tgtcccgggg gccaccgcag cctccaggca gcggggggac ccagccccca |
| | 44401 | atatgtgaga agagcaggtc ccaggctgga gagagcgaag caccatggtg gggagaagtt |
| | 44461 | agactggatc ggggccccta ggggctcccc cggacctgca cggcagccgt cagggcaccc |
| | 44521 | gcaccccatt gctgttcagt gctggccagt gtccaaggcc agggatgtgt gtgtgtgtgt |
| | 44581 | gtgcgtgcgt gcgtgcgtgt gtgtgtgcgt gtgtgcgcgt gcgtgcgtgt gtgtgtgtgt |
| | 44641 | gcgtgcgtgt gcgtgcgtag acgtgtgcgt gcgtgcgtgc gtgcgtgcgt gtgtgtgcgc |
| | 44701 | acgcgcgcag cccagcctca gcactggacc aggcagcctg ggattcctcc aaaactgcct |
| | 44761 | tgtgagtttg gtcaaaccgt gaggctctga tcaccgccat ccattcgccc cctcctgccc |
| | 44821 | ccctcatcac cgtggttgtt gtcattatcg agagctgtgg agggtctggg aggtcatccc |
| | 44881 | acctgccagc taaaccgtga ggctgccgca atcgcactga tgcgggcaga cccgagacgc |
| | 44941 | tgtgccggag acgaaggcca gcttgtcacc ccgccagagc ggcagtcggg ccacaagcat |
| | 45001 | catccaagca gtggttctct gagcccgacg gggtgatgca aaggagccag gagacacctg |
| | 45061 | cgcgtccaag ctgggggacc ccaggtctgt tatgccggac agtaaacacg ttcagctccg |
| | 45121 | gagggagagg gttcccctac cttccagggt ttctcattcc acaaacatcc aaagacaatc |
| | 45181 | cataccgaag gcgatccgtg cctttgctcc tgagacgtgc ggaagcacag agatccacag |
| | 45241 | acactgtctc ccaggatcct atgtatgtaa aggaaccgaa gtcccaggct gtgtgtctgg |
| | 45301 | taccacatcc cacggaacag gctggactga ttttcaccaa atgtagcaga aacgttaagg |
| | 45361 | agtatcagct tcaaaatatg agggccagac atgtctgaga agtcccttcc agaaaagtcc |
| | 45421 | ctttggggtc cttccccaga gttgctgaaa cagagaaccg gaagggctgc agagctgaac |
| | 45481 | ttaaacaact ggatcgcaaa ggtccgtctc atcagagcga tggtttttcc agtggtcatg |
| | 45541 | tatggatgag agagttggac cataaagaaa gctgagcgcc gaagaatcga tgcttttgaa |
| | 45601 | ctctggtgtt ggagaagact cttgagagtc ccttggactg caaggagatc caaccagtca |
| | 45661 | atcctaaagg aaatcaatcc tgaatattca tgggaaggac tgatgctgaa gctgaaactc |
| | 45721 | caatactttg gccacttgat gcaaagaact gactcactgg aaaaaccctg atgctgggaa |
| | 45781 | aggttgaagg caggaggaga aggggtcgac agaggatgag atggttgggt ggcatcaccc |
| | 45841 | acccatggac tcaatggaca tgggtttgag taaactctgg gagttggtga tggacagaga |
| | 45901 | atcctggcat gctgcggtcc atggggtcat agagagtcag acacaactga gcgactgaca |
| | 45961 | gaactgaagc aactggcaag ccggagggta ggtgccggct gcgatgagcg ggaacgtgca |
| | 46021 | acctgccacg tggagctctt cctacaccca gagtcctgac ggcactggga ccctagccct |
| | 46081 | ccacggcctc tccagggcca-cgagacaccc tcacagagca gagaagcgga acagagctgg |
| | 46141 | tgtgcagaac caggccccgg gggtggggcg gggctggtgg gcaggcttta gtgagaagcc |
| | 46201 | cttgagccct ggaaccagag cagagcagaa cagttggcag aggcccccct gggagaggcc |
| | 46261 | ccccgcccag agtaccggcc ctgggccctg ggggagaggg cggtgctggg ggcagggaca |
| | 46321 | gaaggcccag gcagaggatg ggccccgtgg gacggggcgc accaaaacag cccctgccag |
| | 46381 | caaggggaag ctggggcact ttcgaccccc tccaaggagg agcccacacc agcgcatctg |
| | 46441 | cccaaggtgc ccttggccct gggggcacat gaggcccagg ccaggccagg gggcccatga |
| | 46501 | ggcccccagg ggtcagtgca gtgtccccag gcagccctgg cctctcatcc tgctgggcct |
| | 46561 | ggcctcttat cccgtgggcg cccacggcct gctgcccccg acagcggcgc ctcagagcac |
| | 46621 | agccccccgc atggaagccc cgtcaggaaa gagcccttgg agcctgcagg acaggtaagg |
| | 46681 | gccgagggag tcatggtgca gggaagtggg gcttcccttc gatgggaccc aggggtgaat |
| | 46741 | gaccgcaggg gcggggaacg agaagggaaa ccagctggag agaaggagcc tgggcagacg |
| | 46801 | tggctgcacg cacagcgctg accctgggcc cagtgtgcct ttgtgttggg ttttattttt |
| | 46861 | aattttgtat tgagatgcta tttatctcgt ggagcttttg ccgccctgag attttgtacc |
| | 46921 | cgtggctggt gtccctcttg cctcaccccg gcctctgtag cagggcagac acggcgcaac |
| | 46981 | ggggcagggc gtgcccagga ggcactgtca ttttgggggc agcggcccca caaggcaggt |
| | 47041 | ctgccttcct cccctcttac aggcagcgac agaggtccag agaggtgagg caagctgccc |
| | 47101 | aatgtcacac agcacacggg cgcagtccca ggactgtaga aatcccggga ctagacaggc |
| | 47161 | accagagtgt cctgtgtttt taaaaaaacg gcccaagaga agaggcaagt ctgcaaggcg |
| | 47221 | tcccgggaag gcagcagggg cttggctcgg tctcccccaa ggaggccagc tcctcagcga |
| | 47281 | ggttcctaag tgtctaacgg agccaagcct gaaccaaggg ggtcacgtgc agctatggga |
| | 47341 | cactgacctg ggatggggga gctccaggca aagggagtag ggaggccaag gaggagagag |
| | 47401 | gggtgcacag gcctgcaggg agcttccaga gctggggaaa acggggttca gaccacgggg |
| | 47461 | tcatgtccac ccctccttta tcctgggatc cggggcaggt attgagggat ttatgtgcgg |
| | 47521 | ggctgtcagg gtccagttcg tgctgtggaa aaattgtttc agatcagaga ccagcgtgag |
| | 47581 | gtcaggttag aggatggaga agaagctgtg aaaaggtgat ggagagcggg gggacggtcc |
| | 47641 | tcggtgatca ggcaccgaga tcgcccatgg aatccgcagg cgaatttaca gtgacgtcgt |
| | 47701 | cagagggctg tcggggagga acaggcactg tcatgaactg gctacaaaaa tctaaaatgt |
| | 47761 | gcaccctttt cggcaatatg cagcaagtca taaaagaaaa cgcatttctt taaaattgcg |
| | 47821 | taattccgct tttaggaatt catctggggg cgggggaaca atcaaaaaga tgtgaccaaa |
| | 47881 | ggtttacaag ccaggaagtc aactcgttaa tgatgggaga aaaccggaaa taacctgaat |
| | 47941 | atccaacaga aagggtgtga tgaagcgcag catggcacat ccaccgcaag gaatcctaac |
| | 48001 | acaaacttcc aaaacaatat ttctgacgtt gggtttttaa agcatgcgtg cactttcaaa |
| | 48061 | agcttgtcag aaaacataga aatatgccaa taatgtgtct ctagccaaat tttttaattt |
| | 48121 | ttgctttata attttataaa gttataattg tatgaaatat aatgataaaa ttataaacta |
| | 48181 | taaaaaagtt atgaaaatgt tcacaagaag atatacatgt aattttatct tctacaatac |
| | 48241 | tttttaatac cagaataacg tgcttttaaa aaagattgag cacagaagcg tataaagtaa |
| | 48301 | aaattgagag tttctgctca ccaaccacac gtcttacctt aaaacccatt ctccagcgag |
| | 48361 | agacagtgtc atgtgggtct gtacacttct ggcctttctc ctaggcatgt atgtccctga |
| | 48421 | aaactcacac acacggctaa tggtgctggg attttagttt tcaaaacgga ctcatactct |
| | 48481 | gcctatgagc ctgcaactat ttattcagtc tgttgagatt ttctatatca gcccacatgg |
| | 48541 | atcccgcatg ttctctgaat ggctctgtat gaattcaaag tttggaagaa gcagcgtgtc |
| | 48601 | tttaatcatt cgcctattaa tggacgtttg gggtgtttcc actacaaaan nnnnnnnnnn |
| | 48661 | nnnnnnnnnn nnnnnnnnnn nnnnnnnnnn nnnnnnnnnn nnnnnnnnnn nnnnnnnnnn |
| | 48721 | nnnnnnnnnn nnnnnnnnnn nnnnnnnnng atacaattcg agctcggtac cctggcttga |
| | 48781 | actatatgaa cagagaacga tgagaacagt ttctcaaact tggaacagtt aacattttgg |
| | 48841 | gctaaatgat tcttttttgt gtggagttgg cctatgaata gaggatatta gcagcatcat |
| | 48901 | ttaaccttta ctcactacat acctgtagca actacatcct ctccatttgt gtcaatcaaa |
| | 48961 | actgtctccg gacatggaca agtgtgcccc tgggatgggt ggaatgacct tttgttaaga |
| | 49021 | accactgggt cagagattca tagatttttg tcttgttgac tttttaaaaa tacatcttgg |
| | 49081 | tttttatttt attggtttct gctcttatct ttatgattac cttcctttta cttggggctt |
| | 49141 | ccctgataga ttttcccttc tggctcagct ggtaaagaat ctgcctgcaa tgcaggagac |
| | 49201 | ctgggttcag tccctgggtt gggaggatcc cctggagagg agaagggcta cccaccccag |
| | 49261 | tattctggcc tggaggattc catggagtgt atagtccatg gggtcgcaga gtcggacatg |
| | 49321 | actgagtgac tttcacacac acatatgtcc ctggtagctc agctagtaaa gaatcccacc |
| | 49381 | cgcaatgcag gagaccccgg tccaattcct gggtccggaa gattcccttt tgtttactcc |
| | 49441 | ataagatctt atctggggac aaaactaaca gctatgccag accttctgga catcagggaa |
| | 49501 | cgtgaggggt gtggactgga cagatgtgtg tgttctccca aacacaaaca tacatctgta |
| | 49561 | tacatgtaca tggagagagg gggagggagg ctgtgagtct ccaggggacc gtgcaaccat |
| | 49621 | gtgacattca tggaggcgtt tgcgggtgat cactacacag tttcttcttc tggtttcttg |
| | 49681 | gtcaattgac ttcacaattc caattcctat acttcatttt agactgaggg aattttacac |
| | 49741 | tattgtaaga catatgtata catgagttat gttcagcgcc atgagggctc attttgtgtg |
| | 49801 | tccactttgc ctggaaacaa agttggactg atttacttct aggggtgcct gggggtgttt |
| | 49861 | ctggaggaca ggagcatttg aacccaaggg ctcggtgaag catgagcctc tctgcaggtg |
| | 49921 | gacccaggag gaacgcaagg ccgaggaagg cagactctcc tcctccctaa cccgaggtct |
| | 49981 | ctgctcagaa aagggacaat ataatgacta gaagaaaaga aagaacatca gctgtgggag |
| | 50041 | gtttgttctc tggagcagat tcacacgttg aggctcatgt gcaggaattc taggtgaaac |
| | 50101 | agagcagtca cccatgtgtg ttggaaaatt ttaaattaca tttgcagtta cgactttgtt |
| | 50161 | taagccagac agggtagcac agcaaagtca ccatgtggtc acctgtgttt tgtaaaggag |
| | 50221 | agagaacttg ctggcacatt caggaaaggc cgtgtctcag ctttggaggc acactgagag |
| | 50281 | gccacaagca gatggtgagg accagggtct cgggcagagg gatcaattca ctgctcttca |
| | 50341 | cttttgccac atctgtgtgc tgtccatcct ggccagagta gttcagtctt cagatgctgg |
| | 50401 | agttcccatt ggtagaaatc caatctgggt catttttaaa cctctcttgg ttctacttaa |
| | 50461 | tggttttaaa atctctttgg ctcaagaaaa aaaataaaca taattttaaa gggtggtttg |
| | 50521 | gggccttgac tataaagtac attatctggg ccatttcaga gcatggttga attaatacat |
| | 50581 | ttcgtgctta ctatagctcc tattttcttg attctttaca ggtaattttt gttaggaatc |
| | 50641 | gggtactgtg aatattttct tgttgaatac gggatctttg tattttttcc taattttttt |
| | 50701 | ttttttttca tttttggttt taccttcagg aaagtcacta ggactcagga aagtcctttg |
| | 50761 | tccgcctgtt atttcagtct cttacctggg gccagggcag cgtttcctct gggctaagtt |
| | 50821 | tccccacaac cggggccagt tctcctcact cttcaccctg aggccttaat gaggagctcc |
| | 50881 | cctgcgtctg agcagccggc cctcctgtga cgtgcgtgtg tctctggcca tcggcgtccg |
| | 50941 | gtgtccttgg aggttccgtc ctcccttcgc tcactgtgcc ccgcactcga gctctcaggc |
| | 51001 | tccaagcagt gtccgcagtg tgcagaccct ctgtgtagct ctctcctcct caggactctt |
| | 51061 | ccctctagat gtgtgttttc ttttggctcc ttggacctcc gctctgaacg caggcctggt |
| | 51121 | gctgagtgtg atctctggag ggaagcctgg gaggctggac gggtccgccc tgcggtgtgg |
| | 51181 | tgacaggtgt gggctcgggg cggggcctgc acgtcgtcct gacccgagcc gggactgggc |
| | 51241 | tccgggcctc aggcatcact gactgaatct ccctcacaga ggggtcaggg cctgggcggg |
| | 51301 | ggaaccgtct ctgcaatgac agcccctccc agggagggca cagcggggag ctgccgaggc |
| | 51361 | tccagcccta gtgggaggtc ggggagccca ggggagcggc ctgacggccc cacaccggcc |
| | 51421 | cagggctggt tcgttctgtt tctcgagctc aacagaagct ccgaggagct gggcagttct |
| | 51481 | ctgaattcgt cccggagttt tggctgctga gtgtcctgtc agcaccgtat ggacatccag |
| | 51541 | agtccattag cagtggtctc tgtccctctg tctgtccttc atcaggctct ttgtccaggt |
| | 51601 | caccacacgg ccaacaccag gacagtctgg tcccgccagc ccatcgtccc tgcggacgcc |
| | 51661 | cctgtgcagc ctgccgaagg gccgggaggc cgggggaacc gggccaggcc tgtccctgct |
| | 51721 | gtgtccacag tcctcccggg gctggaggag agcgtgagca ggacgggagg gtttgtgtct |
| | 51781 | cacttccccg tctgtctgtg tcactgtgag gattatcact gctgtcagct gactgacagt |
| | 51841 | aatagtcggc ctcgtcctcg gtctgggccc cgctgatggt cagcgtggct gttttgcctg |
| | 51901 | agctggagcc agagaaccgg tcagagatcc ctgagggccg ctcactatct ttataaatga |
| | 51961 | ccctcacagg gccctggccc ggcttctgct ggtaccactg agtatattgt tcatccagca |
| | 52021 | ggtcccccga gcaggtgatc ttggccgtct gtcccaaggc cactgacact gaagtcggct |
| | 52081 | gggtcagttc ataggagacc acggagccgg aagagaggag ggagagggga tgagaaagaa |
| | 52141 | ggaccccttc cccgggcatc ccaccctgag gcggtgcctg gagtgcactc tgggttcggg |
| | 52201 | gcaggcccca gcccagggtc ctgtgtggcc ggagcctgcg ggcagggccg gggggccgca |
| | 52261 | cctgtgcaga gagtgaggag gggcagcagg agaggggtcc aggccatggt ggatgcgccc |
| | 52321 | cgagctctgc ctctgagccc gcagcagcac tgggctctct gagacccttt attccctctc |
| | 52381 | agagctttgc aggggccagt gagggtttgg gtttatgcaa attcaccccc gggggcccct |
| | 52441 | cactgagagg cggggtcacc acaccatcag ccctgtctgt ccccagcttc ctcctcggct |
| | 52501 | tctcacgtct gcacatcaga cttgtcctca gggactgagg tcactgtcac cttccccgtc |
| | 52561 | tctgaccaca tgaccactgt cccaagcccc ccggcctgtg gtctcccctg gactccccag |
| | 52621 | tggggcggtc agcctggcag catcctggcc gtggactgag gcatggtgct ctggggttca |
| | 52681 | ctgtggatgt gaccctcaga ggtggtcact agtcctgagg ggatggcctg tccagtcctg |
| | 52741 | acttcctgcc aagcgctgct ccttggacag ctgtggaccc gcagggctgc ttcccctgaa |
| | 52801 | gctccccttg ggcagcccag cctctgacct gctgctcctg gccacgctct gctgccccct |
| | 52861 | gctggtggag gacgatcagg gcagcggctc ccctcccgca ggtcacccca aggcccctgt |
| | 52921 | cagcagagag ggtgtggacc tgggagtcca gccctgcctg gcccagcact agaggccgcc |
| | 52981 | tgcaccggga agttgctgtg ctgtgaccct gtctcagggc ggagatgacc gcgccgtccc |
| | 53041 | tttggtttgt tagtggagtg gagggtccgg gatgactcta gccgtaaact gccaggctcc |
| | 53101 | gtagcaacct gtgcgatgcc cccggggacc cagggctcct tgtgctggtg taccaaggtt |
| | 53161 | ggcactagtc ccaccccagg agggcacttc gctgatggtg ttcctggcag ttgagtgcat |
| | 53221 | ttgagaactt acatcatttt catcatcaca tcttcatcac cagtatcatc accaccatca |
| | 53281 | ccattccatc atctcttctc tctttttctt ttatgtcatc tcacaatctc acacccctca |
| | 53341 | agagtttgca ttggtagcat atttacttta gcacagtgtg cctcttttta ggaaactggg |
| | 53401 | ggtctcctgc tgatacccct gggaacccat ccagaaattg tactgatggc tgaacccctg |
| | 53461 | cgtttggatt cttgccgagg agaccctagg gcctcaaagt tctctgaatc actcccatag |
| | 53521 | ttaacaacac tcattgggcc tttttatact ttaatttgga aaaatatcct tgaagttagt |
| | 53581 | acctacctcc acattttaca gcaggtaaag ctgcttcgca tttgagagca agtccccaga |
| | 53641 | tcaataaaga gaatgggatg aacccaggat ggggcccagg ggtcctggat tcagactcca |
| | 53701 | gccgtttagg acagaacttg actaggtacg aagtgagcgg ggtggggggg caatctgggg |
| | 53761 | ggaactgtgg cacccccagg gctcggggcc atccccacca catcctggct ttcatcagta |
| | 53821 | gccccctcag cctgcgtgtg gaggaggcca gggaagctat ggtccaggtc atgctggaga |
| | 53881 | atatgtgggg ctggggtgct gctgggtcct aggggtctgg ccaggtcctg ctgcctctgc |
| | 53941 | tgggcagtga taattggtcc tcatcctcct gagaagtcac gagtgacagg tgtctcatgg |
| | 54001 | ccaagctatt ggaggaggca gtgagcactc ccacccctgc agacatctct ggaggcatca |
| | 54061 | gtggtcctgt aggtggtcct ggggcttggg ccgggggacc tgagattcag ccattgactc |
| | 54121 | tcagaggggc cagctgtggg tgcagcggca gggctgggcg gtggaggata cctcaccaga |
| | 54181 | gccaaaataa gagatcaccc aacggataga aattgactca caccctttgg tctggcacat |
| | 54241 | tctgtcttga aatttcttgt ggacaggaca cagtccctgg ataaagggat ttctatcttg |
| | 54301 | cgtgtgcaat agagctgtcg acacgcttgg ctgggacatg taatcctttg aacatggtat |
| | 54361 | taaattctgt tcactaacat ctgaaaggat ttttgcatca ataaacctaa ggtatattgc |
| | 54421 | cctgtcattt ccttgtcttg tagtgtctct gagtaggctg gaaggggtaa ccagcttcac |
| | 54481 | aaatcgagtt aggaaattcc cttattcttc cactgtctaa tagactttca taagattagt |
| | 54541 | gttaattcct ctttaaatcg ctgctataat catcactgtg gccaccggta ctgaattttt |
| | 54601 | tgttaggatg atttttaaac aagcatttta atgatttttc cttttatttt cggctgtgct |
| | 54661 | gggtctcgtt gctgtgtgcc ggcgttctct cgctgtggcc agtgggggcg ctgctctcgc |
| | 54721 | gttgcgaagc tcgggcttct gactgcagtg gcttctctcg ttgcagagcg cgggctccag |
| | 54781 | ggcgctcagg ctcgcgtggc tgcggcacgt gggctcagta gtcctggggc acaggtgcag |
| | 54841 | cagcctctca ggacgttttg ttcccagatg gtgggtcggt cgaaccggtg tcccctgcgt |
| | 54901 | tgcaaggtgg attcttcacc gctggaccac cagcgacgtt ccctggaggt ttttaattat |
| | 54961 | ggatttaagc tctcattaga tgtctcctca catttcctat ttctttttga gtcagtttga |
| | 55021 | tactttgttt gtgtctgtaa gtttgtccat tttatccaag tcatctaatg tgttgataga |
| | 55081 | caattattgg ttagtcatct aattgttggt ttacaatttt gagagcattg tcctgcaatt |
| | 55141 | ccttctatct gcaagattgg taataatatc tcccaagagg agtcacaaac tgaaatgaga |
| | 55201 | ttanatacag gctttttttt taaaagaatg aacttatgtt gttgcctttc tcatagatct |
| | 55261 | tacttcttag catgactgta cttactgact ggggcgtttt catgtctgtg tggagagcta |
| | 55321 | ccattagtac ttcttatcgc ccaaagacat cgggctcctg ggcacagtga aaacactcct |
| | 55381 | ttctgtggct attttgcaaa atatggccta gcctagcgtc ataagggatc acagctgaca |
| | 55441 | actgctggaa cagagggaca tgcgaagcaa cgtgagggct ggaacctgga gggtcctctc |
| | 55501 | tggggacagt ttaaccagct ataatggaca ttccagcatc tgggacatgg agctgtgaac |
| | 55561 | tggaccaatg actgtcattt ttggaagaga aatcccagga gagaagggtc caggggaatc |
| | 55621 | tgaggccgca tgcagtgcct caggacaggg gacaccttct ccagcagagc aggggggccc |
| | 55681 | gcccaggccg cctgcagtga ttccaccagg aggagatgca tccctgcaga cctctgacag |
| | 55741 | cacggccctc tcctgagaca cagggtcaca cccggggccc tggaaccctt tgagacccta |
| | 55801 | aacctttcct ttcctgacca ccctgacagc agtctagctc agaacagaca tcttcatttt |
| | 55861 | cagcaggaaa atccttttcc tcgtttgagg gagcgactgg caccggagga gctgagtctt |
| | 55921 | ttaaacacag gctgcctgaa cctcagggat gacctgcagc tgctcagagg aggctggagt |
| | 55981 | gtgatagctc actctaatgt tactaaaagg aacatattgg acaccccctc tctgaaaaat |
| | 56041 | ttccctcctg cctctcatct cttagtccac tttatcgccg ttttactgct tttctattta |
| | 56101 | ctactcttaa cgccaaccta tcttatttcc cctcccagtt taacacggtt ttccctccac |
| | 56161 | ccgctctctt taatctcaga agattctgcc tattcctcta ttatcacacg cccctacttt |
| | 56221 | ttattttttt tcttacccgc cttttattcc ctcccctcct cactctctat ttaattacat |
| | 56281 | cttaactaca ccgcctgcgc tatcttcgaa tgtatccaaa tatttttccc ttatataaca |
| | 56341 | ctccaggccg agcggctaac ttattataat ttctttatag cgcctaccta atttcccttt |
| | 56401 | atttctaatt atctatatat acccatgcaa tttcgnnnnn nnnnnnnnnn nnnnnnnnnn |
| | 56461 | nnnnnnnnnn nnnnnnnnnn nnnnnnnnnn nnnnnnnnnn nnnnnnnnnn nnnnnnnnnn |
| | 56521 | nnnnnnnnnn nnnnntgggt gtacgttata gagtaaacgc gcatgaagaa gtgggtcaat |
| | 56581 | ctatggctgt gagaggcaga aaataatatt atcatatata atttatgtta taacacactg |
| | 56641 | aggtggtggg ctcgtagaat agtgcggacg gggagaaagg tgggaaggag aagacacaag |
| | 56701 | agagagatgt tcgcctcgcg ggatggatgg gcggagggat agaagaataa aaagaggaga |
| | 56761 | ggtatagagg ggggcggggg gcataacgtg tggtggggta aatagtaggc ggtaattatg |
| | 56821 | aaaaaaagaa agacgggggg ggcggtaaca tagaatacgc aaaaaagtca tatactgaac |
| | 56881 | ggggattagg gagaagaggt ggggggcgtg gggtgcgggg gaaagaggtg tgtgtataat |
| | 56941 | tggtatggag tgttatttga atatatatta atgtaatagg gagtgtaatt agtgaaattg |
| | 57001 | tgggagtatt atattggggt gtgggggaca tggcaaagtg atgatcggga taaaaaaagt |
| | 57061 | aaagcaagag gggaggggaa aataaggggg gggagaaggt cgaagaaaat aagaggaaga |
| | 57121 | agaaagaacg ggggtggcgg gcgggggggg cgccgctctt gtatctggct tttttgttgt |
| | 57181 | gtcggtggtt gttcgcgtct tgttgggtcc ggggcgggtg tgcggaaaaa aaaaaaggcg |
| | 57241 | ggaggcccgg ggcccggtca cgcggcaccc ccgcgggtcc ctggcttctc cttcggcagc |
| | 57301 | tccgggggtc ggtgagcctg cgccctccgg gccgccggcc cgagctgtgt gcgccctgga |
| | 57361 | gaatcggagc cgctgtggca gcacgcggag ggcgcgcgca agggccacgg gacggacctt |
| | 57421 | caaaggccgc ggcggagcgc ggcaagccga accgagggcg gtctggcgat cggccgagcc |
| | 57481 | ctgctccccc ctcccgcgtg gccccagggt cgcgggtgga ctggggcggg tacaaagcac |
| | 57541 | tcacccccgt cccgccccca gaaagcctcc caggactctc acagagcacc cgccaggagg |
| | 57601 | catccggttc ccccctcggc tcagttcagt tgctcagtcg tgtccaactc tttgcgaccc |
| | 57661 | catggactgc agcaccccaa gcttccctgt ccatcaccaa ctcccggagt ttactcaaac |
| | 57721 | tcatctattg agtcagtgat gccatccaac cgtctcatcc tctgttgtcc ccttctcctc |
| | 57781 | ccactttcaa tctttcccag catcagggtc ttttcttatg agccagttct tcacatcagg |
| | 57841 | tggtcagagt attggagttt cagcttcagc atcagtcctt ccaatgaaca ctcaggactg |
| | 57901 | atttccttta ggatggactg gctggatgca gcgccagaca ccgaccgcgt ttaccccgtg |
| | 57961 | tgtcctttcc aatggctgtc ccctgcgggc ctaggggcat tggtgcgggt ttgaatcctg |
| | 58021 | tggccttgaa ttttacgcct tagttccagg tccagggcag ggccatccgg attcaggatg |
| | 58081 | cttcccagcc cttcaggaat ggcaggtttt catggtcctt tctgagtgag ttctgagtgg |
| | 58141 | tcatattggt gcccttggca gggagggctc ctgactttcc tatcttcaca tcactgtccc |
| | 58201 | caacccccaa gagaggcctc ttggcccagg gactgcaggg aggatgaagt caggagcaga |
| | 58261 | agcatggggt agggggctca ggtgggcaga ggaggcccct ctgtgaggag gaacggcaag |
| | 58321 | cgaggaggga acaggggcac cggcagtgcc tggcaagctg ggtgatgtca cgactacgtc |
| | 58381 | ccgaccacac agtcctctca gccagcccga gaagcagggc cctcccctga cccccatctg |
| | 58441 | ggcctgggct tcagttttct cctccctgca atggggtgac tgtttgcctc caggagaggg |
| | 58501 | gagcatgtaa aggtggccac tctcttctgg cagacatgcc aggcctgggc cagcctccac |
| | 58561 | ccctttgctc ctgcagcccc tgctgacctg ctcctgtttg ccacaccggc ccctcctggg |
| | 58621 | ctgatcaggg cccccctcct gcaggaagcc ctctgggaca agcccagctt gctgtaactg |
| | 58681 | tggctttcca ctgtgacctg caacgtggga ggctgttact taaaactccc atgactggtg |
| | 58741 | gattgccggt ccccagaaca aggccacgca tccctggagg ccctcgagac catttaaggt |
| | 58801 | agttaaacat ttttacttta tgcattttca tgtgtatcag aaagaaaaaa aatgtatcat |
| | 58861 | cagttcatca aatccatgat ttcttgacca atattgctaa gatgaggctg aaataggcat |
| | 58921 | ttccattttt aaaaaactga atcactctga agaaacagat ggcaggcttc cctggtggtc |
| | 58981 | cggtggttaa cagtccatgc ttccagtgct gggggcatgg gttcgatccc tgaaaatttt |
| | 59041 | aaaaaggaag aaaaagatgg ctcccccgtc cctgggattc tccaggcaag aacactggag |
| | 59101 | tgggttgcca tttccttctc cagtgcatga aagggaaaag ggaaagtgaa gtcgctcagt |
| | 59161 | cgtgtgcgac tcttagcaac cccatggact gcagcctacc agactcctcc gtccatggga |
| | 59221 | ttttccaggc aagagtactg gagtggggtg ccattgcctt ctccaggcaa acggcctgct |
| | 59281 | actgctactg ctgctaaatc gcttcagtcg tgtccaactc tgtgcgaccc catagacggc |
| | 59341 | agcccaccag gctcccccgt ccctgggatt ctccaggcaa gaacactgga gtggggtgcc |
| | 59401 | attgccttca gcctgctgct gctgctgcta agtcgcttca gtcgtgtccg actctgtgtg |
| | 59461 | accgcataga cggcagccca ccaggctccc ccgtccctgg gattctccag gcaagaacac |
| | 59521 | tggagtgggt tgccatttcc ttctccaatg catgaaagtg aaaagttaaa gtgaaattgc |
| | 59581 | tcagtcgtgt ccgactctta gtgacccaat ggactgcagc ctaccagggt cctccatcca |
| | 59641 | tgggattttc caggcaagag tactggagtg gggtgccatt cggcctaggg agtgagaaat |
| | 59701 | cacggctgtc ttccctcttc tcgccctcta ggggtctctg tggagcctcc ctggagaggc |
| | 59761 | cgcggcggct ccggggactg gagggggagg gggggttgag tcagccggtg gccctcccct |
| | 59821 | cgctgcccgt ctcctccctt tttaggcaca agctgggcgc cctttttagg cgcagcctca |
| | 59881 | ccctgcgggc cactgcccgt gtttcggctc cccggagata aaacagattg cctgcacccc |
| | 59941 | gggtcatcac aaggattgta tgaccgtttc ccagtgtgct caccaccctc cctctgattc |
| | 60001 | tcagagacgc gccctcgcct caggaggctg ctcatcccag gccaaggggc ggcgtggggt |
| | 60061 | ccccagcgcc ccgcacagac actgccttct gaccacctcc tcccaacagc ttacctgcca |
| | 60121 | agaaggcctc ctgacccctc atcctgcccg gtggtttgga gaaagcctca tctggcccct |
| | 60181 | ccttctcggg gcctcagttt ccccctctgt gaactggcgg attctgccaa gctgacgtcc |
| | 60241 | tggccagccg cctccccgtg gccagtgtcc cccgggacac agctgaatgt ccctgctcgg |
| | 60301 | gatgcacctt cccaagttgg cctgtcagga ggcgggggcg agcagggaaa cccgactcct |
| | 60361 | ctcagacggc ccatcgcatt ggggacgctg aggcccggag cagcggcacc ctcctggcca |
| | 60421 | gggtcattct cccgccccgc cccgtccctc cgggcctccg agaccgcagc ccggcccgcc |
| | 60481 | ccgggaagga ccggatccgc gggccgggcc accccccttc cctggccgcg ggcgcggggc |
| | 60541 | gagtgcagaa caaaagcggg gggcggggcc ggggcggggg cggggcggag gatataaggg |
| | 60601 | gcggcggccg gcggcacccc agcaggccct gcacccccgg gggggatggc tcgggccgcc |
| | 60661 | ggcctccgcg gggcggcctc gcgcgccttt ttgtttttgg tgagggtgat gggggcggtc |
| | 60721 | gcggggtact attttttcat ttataattgg gtattagcta gcgagtggaa ccacaccctt |
| | 60781 | attccactat agccaatttt tgcgggggca tcttacatta cagactcgcc cgcctcttat |
| | 60841 | ttcggtacag catatcagat cgtctctrta ctcagacact agtgattatt gtctatagta |
| | 60901 | cacaaaaaga acggttgtgt cggcgtaatg gttgcatttt ccctcctcgt ttctcctgac |
| | 60961 | cacctcaatt acaccaacac tctactattt aaatcacgta ttgtacgcca ccctccgccc |
| | 61021 | gcgaactaaa agaatgtgca gatattctga agataaaatc gttcattgtt acgccccgcg |
| | 61081 | cgcttcgcgt atattactct tagaacttct tattcgcccg agcagttatt caccccccgc |
| | 61141 | aactagatgt cgccttaata tttgttctaa ccgttrtgga ttctaacgat aggcgggaaa |
| | 61201 | ggtagacatt cgaccgctac gacaactaaa atcgacgagc acaggctatt tatatcgcga |
| | 61261 | ccacacgcgc gcggtataca naccgtaaaa ttatctaaca tcgagagtaa gggcacagag |
| | 61321 | cgaaatacaa gcggcgtggt gggaggtgtg tctgtagtga attcgcacct cgcgccgccg |
| | 61381 | cctctgtgcg tcgnnnnnnn nnnnnnnnnn nnnnnnnnnn nnnnnnnnnn nnnnnnnnnn |
| | 61441 | nnnnnnnnnn nnnnnnnnnn nnnnnnnnnn nnnnnnnnnn nnnnnnnnnn nnngatataa |
| | 61501 | tattaataaa cagcggatag atgtgtgtaa gggaggaggt gcataagaga ttaaagagag |
| | 61561 | gcgggcggag agaaatagag tagaggagga tgagagaaaa aagaaagcaa gcgtaggtac |
| | 61621 | aacggcgggt gggtagtatg ataaagtgag tgtatatatt tgagtaaagg aagggtagat |
| | 61681 | ggagtataaa gaagtaagga gaggagaggg cggcggagag agagagtgca aagaaaataa |
| | 61741 | gtgggcaaag gcggggtggg tgagaagcag tagaagagaa gatagagaag ggggaaaaag |
| | 61801 | aggaaaatga ggattagaac aagtaggaca ggatagatgt gaaaaatgag atcaggtcaa |
| | 61861 | ggtggagaaa aagtagaaac tggggcgtga ttgtaaaaaa gggaggccgc gatggggcag |
| | 61921 | caccataagc gaagagatga attaatgaaa gcaaggcagg gagaatcaaa tgagttgggt |
| | 61981 | ggaggaagga ggctgtgact tccttcgctg ccggaaagag aactagaata gcctcgggct |
| | 62041 | gtggggggag gtaaagataa agtgacttct gggccctggg ggaggcccag gagtttctac |
| | 62101 | cgagctgagc tgggtgcctc tcccaaatgc ccaaccccct gagagtcgac gggagagcac |
| | 62161 | agcctggcca aacctgggca gggcacacgt gtccttcacc ccacagtggt cacgagccca |
| | 62221 | gcgtggtccc tgcgtctggc gggaaacaca gaccctcaca ccccacacaa gggtccggcc |
| | 62281 | gctttcaaat aacagcagcc gtgccctctg ggccggtgac ccggacacag agagatgaag |
| | 62341 | tccgcatctc tcagagtgcg ctgtcctccg cccggtcagg cccgggtccc ctgcttctct |
| | 62401 | gaggtcacca ggagggattg catgtgggtc tcagggacac aggttcagtg atgtgacaga |
| | 62461 | gggtagtggg tcccagcagg gccggtcttt ggacccgttt ttctgaaaag ccagttggcg |
| | 62521 | acctggggtc acagcaaagc tgatcctgtt tggccaggag tctcccagtg acggcctccc |
| | 62581 | ccagaacatc gggcccagtg ggggctccag ggggtagact tgcctcccag ctcacgcccg |
| | 62641 | tgtcttgaca agtccatgat ttggtaaaat taatttgtgt tggatggagt tgatttagtg |
| | 62701 | gtgtgtgagt ttctgtggcg cagcaaagtc aatcagttac gcatacacat gtatccagct |
| | 62761 | cttcctacga ttctgttccc atataggtca ttatggggtg tcaggtagag cttcctgtgc |
| | 62821 | tacgcagtac ggccttattc agttcagctc agtcgtgtcc gactccttgt gaccccatgg |
| | 62881 | actgcagcac gccaggctcc cctgtccatc accaactcct ggagcttatt caaactcatg |
| | 62941 | tccatcgagc cggtgatgcc atccaaccat ctcatcctct gtcgttccct ctcctcctgc |
| | 63001 | cttcagtctt tcccagcacc ccctagagaa gggaatggca aaccacttcg gtattcttgc |
| | 63061 | cctgagaacc ccatgaacag tacggaaagt ccttattagt tttctatttt atatatagca |
| | 63121 | gtgcacacgt gtcagcccca atctcgcaat ttatcacccc cctccgccgc cgattggtag |
| | 63181 | tcatgtttgt tttctacatc tgcgactcta tttctgtttt gtaaacaagt tcatttacac |
| | 63241 | cactttttta gattctgcac atacgtggca agcccacagc aaacatgctc aatggtgaaa |
| | 63301 | gactgaaagc atttcctcta agatcaaaaa caagacgagg atgtccactc actccgtttt |
| | 63361 | tactcaacac agccctgaac gtcctagcca tggcaatcag agaagagaaa gaaattaagg |
| | 63421 | aatccaaatt ggaaaagaag aagtaaaact cactctttgc aaatgacatg acacttatac |
| | 63481 | ccagaaaatc ctagagatgc taccagataa ctattagagc tcatcagtga atttgttgca |
| | 63541 | ggatacaaaa ttaatacaca gaaatctcct gcattcctat agactgacaa caaaagatct |
| | 63601 | gagagagaaa ttaaggaaac catcccacgg catgaaaaag agtaaaatac ctaggaataa |
| | 63661 | agctacctaa agaggcaaaa gacctgtact cagaaaacta taaaatactg acaaaggaaa |
| | 63721 | tcagacgaca cagagagaga gagataccac gctcttggat gagaagaatc gatagtgtga |
| | 63781 | caatgactat actacccaga gaaacataca gattcagtac aacccctatc aaattcccaa |
| | 63841 | tggcattttt cacagaatca gaattagaac aaaaagtttt acaagtttca gggaaacaag |
| | 63901 | aaagatccta aagagccaga gcaatcttga gaaagaaaaa tggagctgga agagtcaggc |
| | 63961 | tccctgagtt ctgactgtgt atacaaagct ggcatgattt ttaacagcag gggtgtaaat |
| | 64021 | gaacttgttc acaaaacaga tggtggggtg ggcttccctg gtggctcagc tggtaaagaa |
| | 64081 | tcctcctgca acgcaggaga cctgggttcg atccctaggc tgggaagatc ccctggagaa |
| | 64141 | gggaaaggct acccactcca gtattctggc ctggaaaatt ccaaggacca tatagtccat |
| | 64201 | gggtttgcaa agagtcggac acgactgagc gacttccaat cctggaaacg tcccattgtg |
| | 64261 | gacggtgaac tggggttgtc caagctcagg gtaaccgttt gctgagtgac tgacactcct |
| | 64321 | tctcatgggt taaaatgtgg ggcccaaggc caggaccaga ccccgcagtc agccaggcag |
| | 64381 | accctgtgca gccccagcga gtgtgtggcc gccgtggagt tcctggcccc catgggcctc |
| | 64441 | gactggagcc cctggagtga gcccattccc tcccagcccg tgagaggctg ggtgcagccc |
| | 64501 | taaccatttc ccacccagtg acagatccgc ctgtgtggaa acctgctctt gtccccaggg |
| | 64561 | aacctggcag gactcaggga gaatgtctca gggcggccac agatcagggg ctgggggggc |
| | 64621 | agggctgggt ccagcagagg ccctgtgccc actccccgga aagagcagct gatggtcagc |
| | 64681 | atgacccacc agggcaccga cgcgtgcttg cacacaggcc gccccctcat ggtgacactc |
| | 64741 | ttttcctgtg gccacatctc gccccctcag gtccctcctg ctccccagct cctggcctgg |
| | 64801 | gaacctcttc cccgccccgg ggacgtcagg gctggtgtcc actgagcatc ccatgcccgg |
| | 64861 | gactgtgctg atcaccagca cctgcacccc ctctcgggtc tcaccaggat gggcaactcc |
| | 64921 | tgcccatcca gcacccagcc tcctgggtac acatcggggg aggagggaga agcctgggcc |
| | 64981 | agacccccag tgggctccct aaggaggaca gaaaggctgc cgtgggccag ccgagagcag |
| | 65041 | ctctctgaga gacgtgggac cccagaccac ctgtgagcca cccgcagtgt ctctgctcac |
| | 65101 | acgggccacc agcccagcac tagtgtggac gagggtgagt gggtgaggcc caggtgcacc |
| | 65161 | agggcaagtg ggtgaggccc gagtggacag ggtgagtggg tgaggcccag gtagaccagg |
| | 65221 | gcccatgtgg gtgaggcccg ggtggaccag agtgagcggg tgaggcccag gtggacaggg |
| | 65281 | cgagcgggtg aggcccaggt ggacagggcg agcgggtgag gcccgggtgg acagggcgag |
| | 65341 | cgggtgaggc ccgggtggac agggcgagcg ggtgaggccc gggtggacag ggcgagtggg |
| | 65401 | tgaggcccgg gtggaccagg gcgagtgggt gaggcccggg tggacagggc gagtgggtga |
| | 65461 | ggcccgggtg gaccagggcg agtgggtgag gcccaggtgg acagggtgag tgggtgaggc |
| | 65521 | ccaggtagac cagggcccag agcaaagccc cggctcagca gtgatrtcct gagcgcccac |
| | 65581 | tgcttgcagg gacctcagcg atggtaaggc agccctgttg ggggctcccg actggggaca |
| | 65641 | gcatgcagag agcgagtggt cccctggaga aacagccagg gcatggccgg gcgccctgcc |
| | 65701 | aggctgcccc aggggccaca gctgagcccc gaggcggcca ggggccggga cagccctgat |
| | 65761 | tctgggttgg gggctggggg ccagagtgcc ctctgtgcag ctgggccggt gacagtggcg |
| | 65821 | cctcgctccc tgggggcccg ggagggacgg tcaggtggaa aatggacgtt tgcgggtctc |
| | 65881 | tggggttgac agttgtcgcc attggcactg ggctgttggg gcccagcagc ctcaggccag |
| | 65941 | cacccccggg gctccccacg ggccccgcac cctcacccca cgcagctggc ctggcgaaac |
| | 66001 | caagaggccc tgacgcccga aatagccagg aaaccccgac cgaccgccca gccctggcag |
| | 66061 | caggtgcctc cctctccccg gggtgggggg aggggttgct ccagttctgg aagcttccac |
| | 66121 | cagcccagct ggagaaaggc ccacatccca gcacccaggc cgcccaggcc cctgtgtcca |
| | 66181 | ggcctggccg cctgagacca cgtccgtcag aagcggcatc tcttatccca cgatcctgtg |
| | 66241 | tctgggatcc tggaggtcat ggcccctctc ggggccccag gagcccatct aagtgccagg |
| | 66301 | ctcagagctg aggctgccgc gggacacaga ggagctgggg ctggcctagg gcaccgcggt |
| | 66361 | cacacttccc ctgccgcccc tcacttggga ctctttgcgg ggagggactg agccaagtat |
| | 66421 | ggggatgggg agaaaaatgg ggaccctcac gatcactgcc ctgggagccc tggtgcgtct |
| | 66481 | ggagtaacaa tgcggtgact cgaagcacag ctgttcccca cgaggcctca cagggtcctt |
| | 66541 | ctccagggga cgggacctca gatggccagt cactcatcca ttccccacga ggcctcacag |
| | 66601 | ggtccttctc caggggacgg gacctcagat ggccagtcac tcatccattc cccatgaggt |
| | 66661 | ctcacagggt ccttctccag gggacgggac ctcagatggc cagtcactca tccattcccc |
| | 66721 | acgaggcctc acagggtcct tctccagggg acgggacccc agatgggcca gtcactcatc |
| | 66781 | catccgtctg tgcacccatc cgtccaacca tcacccttcc ctccatccat ctgaaagctt |
| | 66841 | ccctgaggcc tccccgggga cccagcctgc atgcggccct cagctgctca tcccaggcca |
| | 66901 | gtcaggcccg gcacagtcaa ggccaaagtc agacctggaa ggtgcctgct tcaccacggg |
| | 66961 | aggagggggg ctgtggacac agggcgcccc atgccctgcc cagcctgccc cccgtgctcg |
| | 67021 | gccgagatgc tgagggcaac gggggggcag gaggtgggac agacaggcca gcgtgggggg |
| | 67081 | ccagctgccg cctggctgcg ggtgagcaga ctgcccccct caccccaggt acaggtctcc |
| | 67141 | ctgatgtccc ctgccctccc tgcctccctg tccggctcca atcagagagg tcccggcatt |
| | 67201 | ccagggctcc gtggtcctca tgggaataaa aggtggggaa caagtacccg gcacgctctc |
| | 67261 | ctgagcccac ccccaaacac acacaaaaaa atccctccac cggtgggact tcaccagctc |
| | 67321 | gttctcaggg gagctgccag ggggtccccc agccccagga agccaggggc caggcctgca |
| | 67381 | agtccacagc cataacacca tgtcagctga cacagagaga cagtgtctgg tggacaggtg |
| | 67441 | cccccacctg cgagcctgga gagtgtggcc ctcgcctgcc ccagccgcgg tcagtcggct |
| | 67501 | cagcaaccgc tgtccactcc cagcgccctg gcctcccctg tgggcccagg tcaagtcctg |
| | 67561 | ggggtgaagc taagtcaggg agcctcatcc atgcccagcc cggagcccac agcgccatca |
| | 67621 | agaaatgctt cttccctcca tcaggaaaca ttagtgggaa agacaagagc tggggggttc |
| | 67681 | tggggtcctg ggggatcaga tgaaggggtc tgggagcagc agcagcctca ggcaccccaa |
| | 67741 | aacaaggccc aggagctgga ctcccagggc tgaggggcag agggaaggaa ggcctcctgg |
| | 67801 | ggggttggca tgagcaaagg cacccaggtg ggggctgagc acccctcggc tggcacacac |
| | 67861 | aggcccccac tgcagtacct tccccctcgg agaccctggg ctcccgtctc ccgcctggcc |
| | 67921 | tgccatcctg ctcaccaccc agaaatccct gagtgcggtg ccatgtgact gggccctgcc |
| | 67981 | ctggggagga aggagattca gacagacagg atgccagggc agagaggggc gagcagagga |
| | 68041 | tgctgggagg gggcccgggg aggcctgggg ggcagggggg caggagttct ccagggtgga |
| | 68101 | cggcgctgtg ctatgctcgg tgagcacaga ggccccgggt gtcccaggcc tgggaaccca |
| | 68161 | gcagaggggc agggacgggg ctcaaaggac ccaaaggccg agccctgacc agacctgtgg |
| | 68221 | gtccagaagg cagctgcgcc ctgaggccac tgagtggccc cgtgtcccga accaccgctg |
| | 68281 | aaacatggga cacacgttcc caggcggagc cactcctgcc ttccgggagg ctcccagcgg |
| | 68341 | gctcatcgct ccatcccaca gggagggaaa ccgaggccca gatgacgaac atcccggcga |
| | 68401 | gcaggtcaaa gccagcccct ggggtcccct ctcccggcct ggggcctccc ctctgcaggg |
| | 68461 | tgggaaaccg aggccacaca ggggctccat ggggctgccc tctgccaggc cctggacacc |
| | 68521 | ccgcgggtga cccccgcctc tatcatccca gccctgccag gccctggaca ccccgtggat |
| | 68581 | gacccccgcc tctatcatcc cagccctggg ggacagatgg gaggcccaag cgtggacccc |
| | 68641 | ctggccaccc cctaccccac agccgggagg agccgggagc tggtggccaa gggcctagag |
| | 68701 | gagccagann nnnnnnnnnn nnnnnnnnnn nnnnnnnnnn nnnnnnnnnn nnnnnnnnnn |
| | 68761 | nnnnnnnnnn nnnnnnnnnn nnnnnnnnnn nnnnnnnnnn nnnnnnnnca atatagaggg |
| | 68821 | ggtgggataa agggtaatat gatgtttagg tagttagagt taaattagaa gggtttggat |
| | 68881 | aaagattaat aaaattacaa gcgtacatat cgtgtgagtg tgggtgataa tatttgtgta |
| | 68941 | tgtggggaat agaagtgagt gtgagtagta ttcaagatgt aagtgtgcga atacaggtct |
| | 69001 | gagcgatttg aatggaagtg aaaaaaagcg tgtgtgtgga ggaggcggga gaggaagata |
| | 69061 | gtgtggggga agaaaagaag gctagtgggt aaagaaatat cagtaggcgg ttgacgaaag |
| | 69121 | aagaactagg aagaattaat ataaaaataa agggaggatt aaaaaataaa gagggaggag |
| | 69181 | gtaacggaaa tagttagtta agaaaagaat ggagagtgga ggtaagataa ataagggagt |
| | 69241 | aatgggagtg aggaggaata aataaaaaaa tggtgaggga aaatagagta gaatgagaac |
| | 69301 | aagaatgaaa aagggagtga agggggtgaa aaaaagtgaa gttgaaaaaa gaggaaaaaa |
| | 69361 | aaggagaaga taaaaaaata aaataaaaaa aggaaaaaaa agaaaaaaag aaagaagggt |
| | 69421 | taaaggacga aaagaaggga agagaaaaaa aatagtttaa gtgggggagg gtaaaaaaga |
| | 69481 | attaataaag taaatatggt tgtggtcgaa aaaaaaaaaa aaattgttgt gttgatgaga |
| | 69541 | agaaaagaaa aaagaagaaa gggaaaagca aaaagaaagg agagaaaaag acaaccccac |
| | 69601 | cgcccgggcg catggagggt gaggatggcg cacgcccgcg gatggcacag catcacagca |
| | 69661 | atcctaaaac gttttcagac cggtgcatct tcaccgcgcg cgcgccccgc ccggccctcc |
| | 69721 | tcccgccctg accgcggacc cccacccgca ccggggagcc tacccccacc ccggggacgc |
| | 69781 | tccgccacgc taaggtcagg actgccgtga agacgcgccg gggtgaaaac gttttatctt |
| | 69841 | catgacataa gcgagtggtt ttgaaacagg tttacaaacc ctcgtgaaga cgcaccctta |
| | 69901 | gcgttaggtt ttgttttttt accatgtgac gatgcaacta ttttcttcct ctcttccaca |
| | 69961 | gtggctagtc gcctccagag cgaggggtat ctcttgtaca gagaccctcg gaacatccgg |
| | 70021 | aggtagtttc ccacctaggg gtaaagcgag aaggctcatt acgagggccg gggctcctcg |
| | 70081 | gggaagggca gggccctggc gcagaggctc tgccacctca gtgacacgca gaccacgcgc |
| | 70141 | ggcctgcagg cgccgggctc tgaaagcagg caaagcccga tctgctgaca tcaggggttc |
| | 70201 | cgcagcagcg aaggtctggc ccgcacctgg cccactggca gggggtaagc tctgcctccc |
| | 70261 | gacgacagca ccaagttcag gaagggccac gcagacactg gtgagacacg gcccccccgg |
| | 70321 | agctgcccga gaagctctga ctttgcacta aagatctctg gcgcggtcca aaaatgtaag |
| | 70381 | gcctctcttc cttttatctt aagactttga tatttttacg atgtaataaa taccaagaag |
| | 70441 | ggcttttaat ttcagacaga tgtaggataa tttcccccgt agcccttgct gctttgttta |
| | 70501 | gtaacgaaac tcaaaccaga aataccaaag gaattttcca aagagtttca aaagcgctta |
| | 70561 | tcagcaatca ctagactgct gcatacatca tcactgcccc aaacaatagc ctgcctgtgc |
| | 70621 | cagttactca aagtactact tacttgacga aaacaaatct agtcctaacg tttttacaaa |
| | 70681 | gaaactccac tcttccgcca acttttcaga aacaaccact cgatcacgtg gcaggggacc |
| | 70741 | gtggctggac tgggtgctgg ctccttctgt gaccaggcaa cactgccccc ttctcggcct |
| | 70801 | ccctacgcct cttgacaaat gttcatcagc tgtaaagttc accccacgag ggacccactt |
| | 70861 | ctgctatttc ccacgtacct accccattat aggagttttc tttgtgacag tttctgcatt |
| | 70921 | tttcatggat ttagaggttt acataatcag ggctgctgaa cagcatgaga gacgtggcca |
| | 70981 | caaggtccct cctgcacctt gccgcagggg cagggcgagt tatctggctt gagcgtggtt |
| | 71041 | accatcaggg ggtaaacaca gtttccagga cgtttttgac aagacactga cccggatgcc |
| | 71101 | cccactacca ccgtgcaggt cctgcaggcc tcccagcctc ccaggccctt cccgaggtcc |
| | 71161 | cttcggaact taggggactc ggtctgcccc cctgggtttt ccctgcacca gcttttgccc |
| | 71221 | cctctggacc caggtttccc aaatggaaaa cgaaggtgtg ggtatggaag ctccctgggc |
| | 71281 | tcctctcagc tgtgcctctg catggtgatg acggctgccc atcggggggg gcaggactgg |
| | 71341 | ggcagctgcg gacaccctcc caaggctgct acccccgagt ggtgtggggc gctgtgggca |
| | 71401 | cgctctgctc agcgcacctc ctggaaacca gcgcctgccg tctgcccggg gcaaccggcc |
| | 71461 | cgggagccaa gcaccactgc cgtcagagga gctgctggct gtgagtggac gccagtctag |
| | 71521 | ctctgaaccc tgcccaggcc tcctgaggtc tgaacattgt aaaatcaggc cccggacggc |
| | 71581 | aactgcctct ccctcctgcc gtctggtctc cataaactgc atctcaggac aaatcttctc |
| | 71641 | actcaccagg gctgaaacag aagactgcag ctatctttct caaatctaag gtgtgctaca |
| | 71701 | gggcaagtcg cagaaactgt ctggcctaag catctcatca gatgcctgag acaagagctg |
| | 71761 | tggacgccaa gctggagcca gagctcctcg cgttctgccc acctggcacc gcgttccacc |
| | 71821 | cagtaaacgc aggcttgatt ttcaaaagta ccaccgactc agagccaatg ctaaaccgac |
| | 71881 | cacttttcct gcccattaga ttgggtgaag gtttctttaa tcaatctgcc agtcaccaca |
| | 71941 | tgccgcctct gtgcccacag gctggcgaag acctttctga gctacggcat gtggcaggca |
| | 72001 | gcggcacctc tcttcagtac ggccagctgt caaggggagc gtttctgtga tgatgtgaaa |
| | 72061 | atacattgca tccggccccg tgtttcatga acacgggtga ggaaaggaaa cacacaaagt |
| | 72121 | tctgatgcga ctgacagcac gggtctcata actcaataca agtcagacaa accacaggga |
| | 72181 | gtcacaggga atcccaatag cctcatctag tgtgaccatc atgaggctta atttattcag |
| | 72241 | tgtattcaat cataaagagg gggaaaaatt gtaaaaaaaa aaaaaaagaa agagtgaaat |
| | 72301 | gtgtaatact gaaaactgtt gctaggagaa gcaagcattg gcgtttgtaa ctgctttgac |
| | 72361 | tccccaagac ccacactcgc ctcgctacaa aagggaggca ctgctgctca gtacttgcac |
| | 72421 | acccgaactg cggatttgta atttaaaaat gtgtgtgtgg acacagcaca agccagagac |
| | 72481 | tgccaaaggt tgagggacac tggaagaact taatatactt ggtgcatgct gccagtgaca |
| | 72541 | gtcagtcacc agctgattca atagagtgcc gaaaggtcac cttttaggta aggatgaagg |
| | 72601 | ggttctgggc tcgtttactt gcactaactc agagttagtc cgagatatcc gaagtgccag |
| | 72661 | gtgcctccca tttgctgatg gatctagctc agggacggct gggccctagc catccaaaaa |
| | 72721 | tcaagcattg ttctcccaac ctgtcttctc gctgataatg gaaggtcaga acgcccaccc |
| | 72781 | gcccacctca aagtcaaaga acaccaagcg ggtgagtccc cactaagctc ggtgtttcca |
| | 72841 | atcagcggtt tcaggattcc agctggggca atgagggagg gagcgtgcga gggatccaac |
| | 72901 | acctcgcccc gtgcgcagca agggataacc caacaccccg tttctgtacg tccggctgga |
| | 72961 | gttgtggaac tcagcgcgga cccggggcca ccgcgacccc cgggaccctg gccgcgcggc |
| | 73021 | gcatccccgc tgccgggaca cgggtaagcg tccccaaact gccggacgcg gggcggggcc |
| | 73081 | ttctccgcca cgccccgata ggccacgccc aaggacaagg atggtcgtgc ccagacggcc |
| | 73141 | ggggcgggnn nnnnnnnnnn nnnnnnnnnn nnnnnnnnnn nnnnnnnnnn nnnnnnnnnn |
| | 73201 | nnnnnnnnnn nnnnnnnnnn nnnnnnnnnn nnnnnnnnnn nnnnnnnncg gagggggggg |
| | 73261 | ggcggggcgg gggctgccgc cgcgcgtata ggacggtggt cgcccggcct ggggtccggc |
| | 73321 | cgggaatgac cccgcctctc cccgcatccc gcagccgccc cgccgcgccc tctgccgcgc |
| | 73381 | acccgcctgc gcacccgccg ccctcggccg cggccccggc ccccgccccg tcgggccagc |
| | 73441 | ccggcctgat ggcgcagatg gcgaccaccg ccgccggagt ggccgtgggc tcggctgtgg |
| | 73501 | gccacgtcgt gggcagcgct ctgaccggag ccttcagtgg ggggagctca gagcccgccc |
| | 73561 | agcctgcggc ccagcaggtg agcaagggct caggggaaac tgaggcccga cacagagccg |
| | 73621 | cagcaagaag gatcctactg gtcactcggc tgttggcctg gggtcatcac aggcgggctc |
| | 73681 | tcccaaccca tcccctgagg ccaaggtccc tagaaccccg tgggcagaca ccaaccagcc |
| | 73741 | ctttaaatat ggggaaacca aggtgcttag gggtcagaga tagccctagg tcgcccaacc |
| | 73801 | ctagtagaag ggagggctgt tggagttcct gagtgcccgc tctcccaccc cccgggaggc |
| | 73861 | cccttcctga gcccaagggt gactggtagt cagtgacttt gggcctgccg acctgtaccc |
| | 73921 | cactgggcac cccaccagtc ctgagccaca tttgggctta gtgacggggt cagggatcat |
| | 73981 | gaggatcaat gtggctgagc caggaaggtg ttagaacctg tcggcctgga gttcatacca |
| | 74041 | gcactgccct gggcttttct agacccatgt cccgcctcct gccccacctg cccctgttcc |
| | 74101 | cgcaccccac cagcagcggc aggggcttcg agagggctgt gggctcaccc tatttcaggg |
| | 74161 | atggagccgc taagacctgg ggcacactgc ccgctaggga cccctgaggc accagggccg |
| | 74221 | ggggctctgc ggaggggcag ccgccacccc cagctttgga gtcctctccc gggtgcccag |
| | 74281 | cccgagctga tccggctgcc tcccacgctg tgccccaggg cccggagcgc gccgccccgc |
| | 74341 | agcccctgca gatggggccc tgtgcctatg agatcaggca gttcctggac tgctccacca |
| | 74401 | cccagagcga cctgaccctg tgtgagggct tcagcgaggc cctgaagcag tgcaagtaca |
| | 74461 | accacggtga gcggctgctg cccgactggc gccagggtgg gaagggcggt ccacggctcc |
| | 74521 | cactccttcg gggtgctccc gctattccca ggtgctcctg cacttcccat gtgctcccga |
| | 74581 | ttctccctgg tgctccctct cctcctggct gctcctttgc ctcccaggtg ctcccacttc |
| | 74641 | tccctggtgc tcctgctcct cccggcggct cctgtacctt cggcctgacc tcctccctct |
| | 74701 | acaggtctga gctccctgcc ctaagagacc agagcagatt gggtggccag ccctgcaccc |
| | 74761 | acctgcaccc ccctcccacc gacagccgga ccatgacgtc agattgtacc caccgagctg |
| | 74821 | ggacccagag tgaggagggg gtccctcacc ccacagatga cctgagatga aaacgtgcaa |
| | 74881 | ttaaaagcct ttattttagc cgaacctgct gtgtctcctc ttgttggact gtctgcgggg |
| | 74941 | ggcggggggg agggagatgg aagtcccact gcggggtggg gtgccacccc ttcagctgct |
| | 75001 | gccccctgtg gggagggtga ccttgtcatc ctgcgtaatc cgacgggcag cgcagaccgg |
| | 75061 | atggtgaggc actaactgct gacctcaagc ctcaagggcg tccgactccg gccagctgga |
| | 75121 | gaccctggag gagcgtgccg cctccttctc gtctctgggg gcccctcggt ggcctcacgc |
| | 75181 | tctgtcggtc accttgcccc tcttgctgat gcaatttccc cgtaattgca gattcagcag |
| | 75241 | gaggaatgct tcgggccttt gcacctgacc gcatgagcag aggtcacggc cagccccctt |
| | 75301 | ggatctcagt ccagctcggc cgcttggccg tgacgttcca ggtcacaggg cctgccggca |
| | 75361 | cagaggagca ggcccttcag tgccgtcgag cactcggagc tgctgcctcc gctgagttca |
| | 75421 | ctcagtgtct acgcacagag cgcccactgt gtaccaggcc ctattccacg ttccccagtc |
| | 75481 | accgagcccc cagggctggt ggggacctgc cctcgggtac actgtgtccc gtcacgtggc |
| | 75541 | tttacgtgtg tctctgaggg aggctggcat tgcggtccac ctctcagcac aaacatctgt |
| | 75601 | cccctgggaa gggggtccca tttctgggtg cgagcagccc cctggggtcc gtgtctcctc |
| | 75661 | cttacctggc tcaaggcccc ggctcctggg tcctggacag cagggagccc acccctcggg |
| | 75721 | gctgtggagg gggaccttgc ttctggaggc cacgccgagg gcccaggcgc cgcctccggc |
| | 75781 | cgtcgccctg agggagcagg cccgacgcca gcgcggctcc tctgtgaggc ccgggaaacc |
| | 75841 | ctgcctgagg gtgcgggtgg gcaggtgccc ctgcccccag gctctcctgt gtgagtgaca |
| | 75901 | ctcaccagcc agctctggat gccacccatc cgggttctcc aggaggcact catagcgggt |
| | 75961 | ggggtcccct ccctcccccc tctgtggagg gagggagtct gatcactggg aggctggtgg |
| | 76021 | tccgtacccg cccccccgac tctggacgtg tttactaccc ccgcctgggc tcaggacagg |
| | 76081 | gcattggatg ggaaggacag ggctgggtcc tggccaggct gggggctctg cagggcatgg |
| | 76141 | gtgcccctgt ctcttcttat attccaacgt cactgcaggg gggcgcaaat cttggacccc |
| | 76201 | acttactgat gatctgcatc aggacatagg tcccccctcc tgcagcgggg ggctggccac |
| | 76261 | ggagggcgct ggggaaggcc cctcctccag cccctcggcg aggctcacca ggtgcccatc |
| | 76321 | ctcagccagc agggcgacgc tcgctgggag ggcggagagg gaggcagggc agggctggta |
| | 76381 | cgacccccgc tggggcgggg gggccctcag ccggtcctcc agcacccttg ctgccccccc |
| | 76441 | tcaccgtcag ggggcacctg gccgctctgc ctcaggtggg cggtgagggt cccaaggcca |
| | 76501 | caccaggtgt tcaccagctc ccagcagctg gctgtgggag aggggcagag gtgggcgcat |
| | 76561 | ggcacccgcc ttccccccag accaggatgc tctgccttcc tcccgcccat ctccccagac |
| | 76621 | atctgaagga ctcttgcctc caccatgcag ccccgcctcc accagaagct caggttcccc |
| | 76681 | gccccccctc cccgaagctg caggacccct gaccagcgaa gagatgggac agttggaaca |
| | 76741 | cacgctcccc cagcagcggc acagcagctg tgtggcccag aagagcccgc ctgtttccct |
| | 76801 | caagcaactc cccatggatg tcatcccatg gacaccccct tccccacacc gcctcctcgt |
| | 76861 | tctccccctc caaggcagag ggaacgcacc cccacctgtc tgctaggaca ggggacccca |
| | 76921 | cttacctccg aacatcacct tgataaacat ggccgtggtg gggacagatc cctccgaccc |
| | 76981 | ccaacttccg acctggggaa ggagctgggg tggagctcga ctgcagggtg gggccctgtg |
| | 77041 | ggaggtgtac gggtggagag ggtgatgggt gggtgggctc aagcggagct ccttgctcag |
| | 77101 | tccaggcggt ccctgcagct agtccaggat cctcagcctt ctccccctca ctggatcagg |
| | 77161 | gaagactgag gttccctccc ctgccccccc acccagcttc caagctggtc tctgtggcag |
| | 77221 | tgggagctgc caagaggtct gagcggccag tatccgggta acggggtttg tggagggtcc |
| | 77281 | gggcattccc ggtgcagggc tctagtgggg gctggagcct cgggcccaga gctgtccaga |
| | 77341 | gaccagtgcc ctcccaccgc cgccgcccgc aaggagagac agagctccca ggcggggagt |
| | 77401 | cggaggttcc tggaggggga gcatcctcaa ctctgcaggc ccccttccca ggcgcactcc |
| | 77461 | cggcctcccc gtcttctgtc ccctgctctt gttgaagtat gattggcata cagttcacag |
| | 77521 | ccactcttcg gagtgttctc cacactaagg atacagaaca tgtccctcgt ccccccaaac |
| | 77581 | tcccagccag gctgtcacga agagggaggc ggccgacggg gcagggcctt gcactcctgc |
| | 77641 | gtgtggggtc cacaggggtc gtccccgtgt cggtggcccc ttcctctcac gccaggaggg |
| | 77701 | tccccttgcc tggaggtgcc gtggatccgc tcgctgcctg ctctttgggt tgtttcccgc |
| | 77761 | atggggtgat gatgaagagg ccagtacaga cactcgccag caggtctctg ggtgaacagg |
| | 77821 | catttatttc tctttcctga gggcagatcc tgggagtggg gtgccggacc gtccggggag |
| | 77881 | agtatgcttc tgtttctaag aagctgccgt gttctccagt gtgctgcacc atgtcacggc |
| | 77941 | ccctctgtgc gtctggactc aggagacctc cttctcagcg gccctccccc ccaggtggtc |
| | 78001 | aggccatctg tgcccttctg ggggcagagc tcagcgccgg aggcgggagg aggcccagat |
| | 78061 | cccagcgcag cccaccagcg ttgctctgct tccctcggca ttcatagctg gagaaagggc |
| | 78121 | aaggagcacc ggctgaagcc ccacctggag gacgcacttc gatggcagca ggtgctcaga |
| | 78181 | ggtggccccg ggcagcattc cccagacgca caggccagtg ctttcttccc aggacaccac |
| | 78241 | tgtgtctggg gacccgagtc ctgcagcacg gtcgggagcg gctgtgccca gattccggcc |
| | 78301 | tgcacccttg gctccagcca ccacccctgt ttgtcaaggg gtttttgtct ttcgagccgc |
| | 78361 | cgaggaggga gtcttttgtc tgcagtgtca cagaagtgcc ataaagaggg gcccacagtg |
| | 78421 | ggagctttat aacattggtg cggagggctg taacaggtca gggaggcact tgagggagcc |
| | 78481 | ttctagggcg atggagatgt tctaaaattt ggtctgggta caggctacag agatgtgtgg |
| | 78541 | gtgtgtgtgt gtgtgtgtgt aaaaccctcg agccacacgt gtgaggtctg tgcatgtgac |
| | 78601 | cgtacacagg agacctcggt ggaaagcagc cacctgctct gactgcacct gtggatttcc |
| | 78661 | agctcctgcc ctcaggcggc cctgcggggc ccactggctg acggggagac ggcaccgccc |
| | 78721 | tcccccgctg tcagggtggg ggggctgacg atttgcatgt cgtgtcaggg tccagcggcc |
| | 78781 | tcccttgcgt ggaggtcccg aagcacctgg agcgccgccc gcagaacagc ggactcctgc |
| | 78841 | ctgcctccct gcctctggcc atggcctgcc cgcctctggc cctctttctg ctcggggccc |
| | 78901 | tcctggcagg tgagccctcc caaggcctgg ctcacctagg ggtgtgtaag acagcacggg |
| | 78961 | gctctagaag taaatcgcgg ggaagtaaat cgtagtgggc aggggggatg gtttccgaag |
| | 79021 | gggccctgag ggggacagga gacctggcct cagtttcccc actggtgagt gaccagatag |
| | 79081 | ccagggtacc tttggactct gactctgggg ggctctcaga gactggtctc ctactcagtt |
| | 79141 | tttcagaggg gaagctggtg tggccttgtc actgccctgc agggcctcag ggacaagcta |
| | 79201 | tccctgagga ggtctccagc agtcagtggc cggaggctga gccgatggat atagtaacag |
| | 79261 | cccaggcggc ctcttggggg tggtcagcct gtagccaggt tttggacgag ccgaagtgac |
| | 79321 | ctaagtgatg ggggtctgca gagcaaggga tgagggtggg cagcaggagg acccagagcc |
| | 79381 | caccagccca ccctctgaat tctggaccct tagctgcatg tggctccttg ggaagacggg |
| | 79441 | gcttaagggt tgcccgctct gtggcccaca cagtgctgat tccacagcac tggctgtgag |
| | 79501 | cttttgggag cagattctcc cggggagtct gacccaggct ttgtggggca ggggctggag |
| | 79561 | ggaaggggcc caggccagac ctgagtgtgt gtctctcagc ctcccagcca gccctgacca |
| | 79621 | agccagaagc actgctggtc ttcccaggac aagtggccca actgtcctgc acgatcagcc |
| | 79681 | cccattacgc catcgtcggg gacctcggcg tgtcctggta tcagcagcga gcaggcagcg |
| | 79741 | ccccccgcct gctcctctac taccgctcag aggagcacca acaccgggcc cccggcattc |
| | 79801 | cggaccgctt ctctgcagct gcggatgcag cccacaacac ctgcatcctg accatcagcc |
| | 79861 | ccgtgcagcc cgaagatgac gccgattatt actgctttgt gggtgactta ttctaggggt |
| | 79921 | gtgggatgag tgtcttccgt ctgcctgcca cttctactcc tgaccttggg accctctctc |
| | 79981 | tgagcctcag ttttcctcct ctgtgaaatg ggttaataac actcaccatg tcaacaataa |
| | 80041 | ctgctctgag ggttatgaga tccctgtggc tcggggtgtg ggggtaggga tggtcctggg |
| | 80101 | gattactgca gaagaggaag cacctgagac ccttggcgtg gggcccagcc tccccaccag |
| | 80161 | cccccagggg cccagactgg tggctcttgc cttcctgtga cgggaggagc tggagtgaga |
| | 80221 | gaaaaaggaa ccagcctttg ctggtcccgg ctctgcatgg ctggttgggt tccaacactc |
| | 80281 | aacgagggga ctggaccggg tcttcgggag cccctgccta ctcctgggtg gggcaagggg |
| | 80341 | gcaggtgtga gtgtgtgtgt ggggtgcaga cactcagagg cacctgaagg caggtgggca |
| | 80401 | gagggcaggg gaggcatggg cagcagccct cctggggtag agaggcaggc ttgccaccag |
| | 80461 | aagcagaact tagccctggg aggggggtgg gggggttgaa gaacacagct ctcttctctc |
| | 80521 | ccggttcctc taagaggcgc cacatgaaca gggggactac ccatcagatg nnnnnnnnnn |
| | 80581 | nnnnnnnnnn nnnnnnnnnn nnnnnnnnnn nnnnnnnnnn nnnnnnnnnn nnnnnnnnnn |
| | 80641 | nnnnnnnnnn nnnnnnnnnn nnnnnnnnnn agagggtggg tgggtggaat ttaatatagt |
| | 80701 | ggtgcgcgtg gagcgtgggc ggcgcattta aggcggtcat ctaaaatagt ggataggggg |
| | 80761 | tggtgtgaca ataacgggtg gtggatgtgg tttacggggg gtgcaatagt tctgagtttg |
| | 80821 | ttagtgtctt cttgatgggg ttgcggcgtg tggacctacg ccttgagtat gtgggggggg |
| | 80881 | aaaagcagtg agggtagtag ggatgggaaa tattggtgga ggttctttgt tggtgtattt |
| | 80941 | tttggtatta tgttgggtgg tggagtggtg ggttgggtgt aatttcgctt gcgttatgtg |
| | 81001 | ttttttttct ttttcgtgtc gtgggttggg ttggttggtg ctttgtggtg gtggtgggtt |
| | 81061 | gtggtataaa aaaaaatgtg tggttgtgct cagcttagcc ctataacggt cggctttgtt |
| | 81121 | tcttgtttgt tctgtgggcg tgagcggatg gctcgggcct ccgtgctccg cggcgcggcc |
| | 81181 | tcgcgcgccc tcctgctccc gctgctgctg ctgctgctgc tcccgccgcc gccgctgctg |
| | 81241 | ctggcccggg ccccgcggcc gccggtgagt gcccgccgtc ctccagcccc cccgccccgc |
| | 81301 | cccgccctcc acgccgaggg gcgccggctc gcagagctgg atccaagggg gtgcccggga |
| | 81361 | gtggcccggc gcggcccgtt accccgaaac gctgtctggg tgccccgggg gtgtggtgga |
| | 81421 | tagtgagctt cccgtccctg gaagtatgca agtgaagccg gcgccgggat cgctcgggct |
| | 81481 | ggctggtgag cgggcgggac tcggtcgggc gctagacgca cgccgccagc cccccagctc |
| | 81541 | ccagacctgc ccactccgcg cccgcccggc cgcgatcccg ggtgtgtgtg tgtgttgcag |
| | 81601 | gggagggaca gcgggagtgg ctacagggct cccgactcac cgcagggaca aagacccgcg |
| | 81661 | ggtccccagc tggcgtcagc cgccaggtgt gtggcctcgg tgagcacacc tccaggcggg |
| | 81721 | agggttgagg gaagcgctgt ggggagggca tgcggggtct gagcctggaa gagacggatg |
| | 81781 | ctaccgcctg ggacctgtga gtggcgggat tgggaggcta tggaatcagg aggcagccta |
| | 81841 | agcgtgagag ctccggtgtg gcctggcggg ggtggtaggg gggggacgcc cctgtgtgtg |
| | 81901 | ccagcctgcg tgtgccctaa aggctgcgcc ctcccccact gctggggctt cgggggacca |
| | 81961 | gtcacagcct aggctactgc aggcgcacag ctccccggga gcccggccca cgcgggtgtg |
| | 82021 | ccgctgagcc tccagcctgt cggggcaggg gtggggggca gggatggggt cgttagcggg |
| | 82081 | gttgggggca gacgcccagg cagactctct gggcacagct ccggtgacaa gggaggtctg |
| | 82141 | gcaagcctgg gccccttctg tccagccacg ccagctctgc cctggccagt cttgccccct |
| | 82201 | ggcagtgctg gggatggaag ggggagcggg tacctcagtc tgggggccct gcctcctccc |
| | 82261 | cagccccgcc cggcccccta ggcctagggg cagagtctag gggtcaccct ggggagctgc |
| | 82321 | tgaatccgcg ggtttaggaa ccggagggac ctgggctttt gaaccacgtg gccctaggtg |
| | 82381 | agccctccgg cgcctcggta gccctcaccc ccagccttgt ccaggtgggc gggtgggagg |
| | 82441 | cgacagtgcc cactgctggg ctgaacagcg tctgcaggga ggccaggaga gctgggcaca |
| | 82501 | cggacacgtt ccatcacctg gagctgccac tgtgccactt gtgcggggtc aggcggggtc |
| | 82561 | tgagccgggc tgtcatctgt cacgccacag atatgcaggg ggcactcggg gtcgcctcgg |
| | 82621 | acatgcttat ccctggacgg ctgttggcag ggccgggaag gctctgtaaa tatttatcca |
| | 82681 | tcccagctca cagctttcag ggttgatgaa agccccgccg cccgcccact gtgggggacc |
| | 82741 | ccgccttccc ttctggagcc agcggggtga gggggtgggg gagatggacc tgcctgccca |
| | 82801 | ggagcaggcg gtgtgactct ggcaggtcac ttgacctctc tgagcctcag ggagggcccg |
| | 82861 | ggatggtgtg cggatgctct ctgccttcct cccagcctga ccagtgtcct cccctcgggg |
| | 82921 | tcgcctcctg cccaccgcag agggggtggc tatggggacc tgggccgatg gcaggcaggc |
| | 82981 | cggagagggc atgcccggct cagccgtgcc cagcacttcc cagtccaggg gcccccgcca |
| | 83041 | ctcccagccg ctggctgcct cccattttcc cgattgcagg ttggccccga ggctgaccgg |
| | 83101 | agcctctggc tcagctggga gactgaattc cccaagcaat tcctcaagga tgtgtgaggc |
| | 83161 | tgtggtgtgg tgcctatccg ggagaggtgg ggtgagcgga ctgggcacct ccgcccaggg |
| | 83221 | caggcccagg gagacgctgg ctgacgagca ggcaggcctg caaggaggac gagcagccat |
| | 83281 | ctcaggaatg tgggttttgg agacaagcca cagctggggg ggtggggggg ccatgggtgg |
| | 83341 | ggaggcctga tccccaggtc taggtccagc tctgggctcc ctcgccgtgt gaccctgggc |
| | 83401 | caagacctgg acctctctgg gccccgtctc ttcccctggg aggtggggcg atgcctgctc |
| | 83461 | cccaatcccc cagggctgtg gatgaggcag acgaggtgtg tgctcatccc cacctcactg |
| | 83521 | ccttccagca gccccgggcg gggggggtgg tggggactgg cgcacccagg tgaggatcag |
| | 83581 | gccttggagc tagggagggc cccccagccc caggccagaa aggacacggg gagacagaat |
| | 83641 | gcaggagggc ggcagagcag gggccagcgg tggggaaact gaggccaaga gcctgtggac |
| | 83701 | gatgtgctcc aggaaaggac ctcgctgcct ggggcctgga tcctagagcc tccaggagcg |
| | 83761 | gtgaccatga cgtgggcagg gaaccggagg ccccggcttg caggtggacc cggcgcgagt |
| | 83821 | cactcttcct ctctggccct gagagcttcc ttccagctgc cgctcctgtg ttctaatgtc |
| | 83881 | aagtctggag gcctgggggg caggtggggg ctgactgcca ggtgggggag ggcaggaatt |
| | 83941 | tggcagagca gcgtcccaga gtgggagaag ccagcccatg gaggggactc tctccatgcc |
| | 84001 | tgctgcccca aagggcgtta tagagagagg tcggttaccc cttcgccatg gccccgttcc |
| | 84061 | cattgaacag atgggaaagt ggaggctgag agaaggctgt gacttgccca gggtctccgt |
| | 84121 | ggcatggaac tgggcctgct gagtctcagg ccggggatct cgctgctgca ctgagcacgc |
| | 84181 | caggatgcag gggtctgggc ctggacctag cgcctcgtgg gggcaagaga ggaaggcacg |
| | 84241 | ctgggcctgc ctgtcaccct ccaccccacc gtggcttgtt gctcaggcct tcctgggggc |
| | 84301 | agaggagagg ggagatttca ctcgctggca ggctaggccc tgggctctct ggggctccgg |
| | 84361 | gggaacaatg cagccctggt ctttctgagg agggtccttg gacctccacc agggttgagg |
| | 84421 | aaaggatttc tgttcctcct ggaggtcacg gagccgacat ggggaggagc aggggcaggc |
| | 84481 | ccggggccca catcctcagt gtgagacctg gacgtgtgtc ctcccacctg acgctggggg |
| | 84541 | tggggggtgg gggccggggg ggatccagtg aaccctgccc ccaaattgtc tggaagacag |
| | 84601 | cgggtacttg gtcatttccc cttcctcctc ttcgtttgcc ctggtgggga cagtccctcc |
| | 84661 | cctggggaag ggggacccca gcctgaagaa cagagcagag ctggggtcag gggtgtgctg |
| | 84721 | ggagcgcaga gagcctcctg ctctgcctgc tggtcattcc tggtggctct ggagtcggca |
| | 84781 | gctggtgggg agcggctggg gtgctcgtct gagctctggg gtgcccaggg cctgggagag |
| | 84841 | ttgccagagg ctgaggccga gggtggggcc ctggcggccc ggctcctgcc ccaaatatgg |
| | 84901 | ctcgggaagg ccacagcggc actgagcaga caggccgggc cagacgggcg ctgaggctcc |
| | 84961 | cggcctctcc cccagctccg ctgtgaccct cacctgcggc ccggggtgcc agggcccccg |
| | 85021 | cttggttctg ccgtgtcttt gcaggctgat cccacgggct ctccctgcct ctctgagctt |
| | 85081 | ccgccttttc caggcagggg aaccgcgacc tccaggctgg gacgcgggga gggtgtatgc |
| | 85141 | gccaggtcag aatcacccct ccaccgggag agcgtggtcc aggggccctg gcagggtggg |
| | 85201 | gaccgagcat ctgggaactg ccagccaccc ccacccatgc agaggggaca tacagaccac |
| | 85261 | acggaggctg tgcctccgct gcagcaactg gagaacaccc agccgcggcc aaacataaat |
| | 85321 | aactaaataa taaaagtttt aaagatcgtt acttaaaaaa acaagtgtgc cccagtgatc |
| | 85381 | ggaccccagt tcccggtgcc ctgagtggtg ccggccctgt gctgagcatg gcctggttgg |
| | 85441 | ttcaccccca gatccacact aaagggtggg atcaccccta ctagtcaggt gagcagatgc |
| | 85501 | agggggggag ggcggcagcc cctccatgct ggtgggtggc cgtggtgggt gtcctgggca |
| | 85561 | ggagccagct cacggagctg gagaggacag acctgggggg ttgggggcgc ccaggaagaa |
| | 85621 | acgcaggggg agaggtgtct gccgggggtg ggggtccctt cgaggctgtg cgtgaagagg |
| | 85681 | gcaggcgggc ctgcagcccc acctacccgt ccccggccca aacggcggga gtaagtgacc |
| | 85741 | ctgggcacct ggggccctcc aggagggggc gggaggcctt gggatcagca tctggacgcc |
| | 85801 | agtcagcccg cgccagagcg ccatgctccc cgacggcctc cgctggagtg aggctgcgct |
| | 85861 | gacacccaca ccgctgaccc gggcctctct cccgctcagg atgccccccg ccgccacccc |
| | 85921 | gtgagcagag ggccacagcc ctggcccgac gcccctcccg acagtgacgc ccccgccctg |
| | 85981 | gccacccagg aggccctccc gcttgctggc cgccccagac ctccccgctg cggcgtgcct |
| | 86041 | gacctgcccg atgggccgag tgcccgcaac cgacagaagc ggttcgtgct gtcgggcggg |
| | 86101 | cgctgggaga agacggacct cacctacagg tagggccagt ggccacgagc tggcctttga |
| | 86161 | tctccacctg ctgtctgaga cacgctggag ctggggggag ggcagatccc tatggccaac |
| | 86221 | aggctggagt gtcccccaac tcccgtgccc actgctcaac accccaaacc cacacttaga |
| | 86281 | tgcactccca tgccctccct tgggagcacg gtctccacac ccacctggcc accccacaca |
| | 86341 | cccgtggggc acggccgtta gtcacccacg caacctctgc gggcaccgtg ctgcgggcca |
| | 86401 | ggccctggga ctctcagtga gggaggcaga cacggcccct cctccggggg agcgaggtgc |
| | 86461 | tccccacgcc cggttcagct ctagcaccgc actcgggacc ctcacaggga gggacccact |
| | 86521 | ggggcaggcc aggtgacggc tcgggtgacc tcggcccctg gcgctgagac tacacttcct |
| | 86581 | gcagtgggcg gcgaagatgg gtgtggtgtc ccacgtcgtt gcagcgggga ctcctggggc |
| | 86641 | ctcggaagtg tcctgggcgg ggagcctggg gagcaggaag ggcaggtctt ggggtccaag |
| | 86701 | gcctccccac ggtcaggtct gggagggggc ctcggggctc ttgggtcctt tccgcccagt |
| | 86761 | gcagaccctc gcggccacct aagggcacac agaccacaca aagctgtgcc catgcagtgt |
| | 86821 | ggggagtggt gcgcaccctc agagcacact gggcccacat cacgcacgcc tgccccctca |
| | 86881 | ctgtgcatcc ggggaaactc ctggccccga cagccagcgg ggctgacgct accccgtgag |
| | 86941 | ccagacccag gcccccctca ccgcccctgt cctccccagg atcctccggt tcccatggca |
| | 87001 | gctgctgcgg gaacaggtgc ggcagacggt ggcggaggcc ctccaggtgt ggagcgatgt |
| | 87061 | cacaccgctc accttcaccg aggtgcacga gggccgcgcc gacatcgtga tcgacttcac |
| | 87121 | caggtgagcg ggggcctgag ggcaccccca ccctgggaag gaaacccatc tgccggcagc |
| | 87181 | cactgactct gcccctaccc accccccgac aggtactggc acggggacaa tctgcccttt |
| | 87241 | gatggacctg ggggcatcct ggcccacgcc ttcttcccca agacccaccg agaaggggat |
| | 87301 | gtccacttcg actatgatga gacctggacc atcggggaca accagggtag gggctggggc |
| | 87361 | cccactttcc ggaggggccc tgtcgaggcc ccggagccgg gcccgggctc tgcgtccgct |
| | 87421 | ggggagctcg cgcattgccg ggctgtctcc ctcttccagg cacggatctc ctgcaggtgg |
| | 87481 | cggcacacga gtttggccac gtgctcgggc tgcagcacac gacagctgcg aaggccctga |
| | 87541 | tgtccccctt ctacaccttc cgctacccac tgagcctcag cccagacgac cgcaggggca |
| | 87601 | tccagcagct gtacggccgg cctcagctag ctcccacgtc caggcctccg gacctgggcc |
| | 87661 | ctggcaccgg ggcggacacc aacgagatcg cgccgctgga ggtgaggccc tgctccccct |
| | 87721 | gcccacggct gcctctgcag ctccaacatg ggctcctcct aacccttcgc tctcacccca |
| | 87781 | gccggacgcc ccaccggatg cctgccaggt ctcctttgac gcagccgcca ccatccgtgg |
| | 87841 | cgagctcttc ttcttcaagg caggctttgt gtggcggctg cgcgggggcc ggctgcagcc |
| | 87901 | tggctaccct gcgctggcct ctcgccactg gcaggggctg cccagccctg tggatgcagc |
| | 87961 | cttcgaggac gcccagggcc acatctggtt cttccaaggt gagtgggagc cgggtcacac |
| | 88021 | tcaggagact gcagggagcc aggaacgtca tggccaaggg tagggacaga cagacgtgat |
| | 88081 | gagcagatgg acagacggag ggggtcccgg agttttgggg cccaggaaga gcgtgactca |
| | 88141 | ctcctctggg cacagctggg aggcttcctg gaggaggcgg ttctcgaagc gggagtagga |
| | 88201 | taaaaggtat tgcaccccat gaagcacgtg tgatccttgc ccctagagac aaggctctgg |
| | 88261 | ggctcagagg tggtgaagtg acccacatga gggcacagct tggagaatgt cgggagggat |
| | 88321 | gtgagctcag tgtgccagag atgggagcct ggagcatgcc aaggggcagg gcctgctgcc |
| | 88381 | tgagagctgg cactggggtg ggcagccaag tgcagggatg gagcgggcgc ccaggtggcc |
| | 88441 | tctttgctgc tcagaacgac ctttcccatg tatacctccc agcgccgctg gcattgccca |
| | 88501 | gtgtccttct tgggggcagg agtaccaagc aggcattatt actggccttt tgtgttttat |
| | 88561 | ggacaacgaa actgaggctg ggaaggtccg aggtggtgtt ggtggcggaa ggtggccgct |
| | 88621 | gggcagccct gttgcagcac acacccccca cccaccgttt ctccaacagg agctcagtac |
| | 88681 | tgggtgtatg acggtgagaa gccggtcctg ggccccgcgc ccctctccga gctgggcctg |
| | 88741 | caggggtccc cgatccatgc cgccctggtg tggggctccg agaagaacaa gatctacttc |
| | 88801 | ttccgaagtg gggactactg gcgcttccag cccagcgccc gccgcgtgga cagccctgtg |
| | 88861 | ccgcgccggg tcaccgactg gcgaggggtg ccctcggaga tcgacgcggc cttccaggat |
| | 88921 | gctgaaggtg tgcagggggc aggccctctg cccagccccc tcccattccg cccctcctcc |
| | 88981 | tgccaaggac tgtgctaact ccctgtgctc catctttgtg gctgtgggca ccaggcacgg |
| | 89041 | catggagact gaggcccgtg cccaggtccc ttggatgtgg ctagtgaaat cagtccgagg |
| | 89101 | ctccagcctc tgtcaggctg ggtggcagct cagaccagac cctgagggca ggcagaaggg |
| | 89161 | ctcgcccaag ggtagaaaga ccctggggct tccttggtgg ctcagacagt aaagcgtctg |
| | 89221 | cctgcaatgc gggagacctg gattcgatcc ctgggtcagg gagatcccct ggagaaggaa |
| | 89281 | atggcaatgc cctccggtac tgttgcctgg aaaattccat ggacagagca gcctggaagc |
| | 89341 | tccatggggt cgcgaagagt cagacacaat ggagcgactt cactgtctta agggccacct |
| | 89401 | gaggtcctca ggtttcaagg aacccagcag tggccaaggc ctgtgcccat ccctctgtcc |
| | 89461 | acttaccagg ccctgaccct cctgtctcct caggcttcgc ctacttcctg cgtggccgcc |
| | 89521 | tctactggaa gtttgacccc gtgaaggtga aagccctgga gggcttcccc cggctcgtgg |
| | 89581 | gccccgactt cttcagctgt actgaggctg ccaacacttt ccgctgatca ccgcctggct |
| | 89641 | gtcctcaggc cctgacacct ccacacagga gaccgtggcc gtgcctgtgg ctgtaggtac |
| | 89701 | caggcagggc acggagtcgc ggctgctatg ggggcaaggc agggcgctgc caccaggact |
| | 89761 | gcagggaggg ccacgcgggt cgtggccact gccagcgact gtctgagact gggcaggggg |
| | 89821 | gctctggcat ggaggctgag ggtggtcttg ggctggctcc acgcagcctg tgcaggtcac |
| | 89881 | atggaaccca gctgcccatg gtctccatcc acacccctca gggtcgggcc tcagcagggc |
| | 89941 | tgggggagct ggagccctca ccgtcctcgc tgtggggtcc catagggggc tggcacgtgg |
| | 90001 | gtgtcagggt cctgcgcctc ctgcctccca caggggttgg ctctgcgtag gtgctgcctt |
| | 90061 | ccagtttggt ggttctggag acctattccc caagatcctg gccaaaaggc caggtcagct |
| | 90121 | ggtgggggtg cttcctgcca gagaccctgc accctggggg ccccagcata cctcagtcct |
| | 90181 | atcacgggtc agatcctcca aagccatgta aatgtgtaca gtgtgtataa agctgttttg |
| | 90241 | tttttcattt tttaaccgac tgtcattaaa cacggtcgtt ttctacctgc ctgctggggt |
| | 90301 | gtctctgtga gtgcaaggcc agtatagggt ggaactggac cagggagttg ggaggcttgg |
| | 90361 | ctggggaccc gctcagtccc ctggtcctca gggctgggtg ttggttcagg gctccccctg |
| | 90421 | ctccatctca tcctgcttga atgcctacag tggcttcaca gtctgctccc catctcccca |
| | 90481 | gcggcctctc agaccgtcgt ccaccaagtg ctgctcacgt tttccgatcc agccactgtc |
| | 90541 | aggacacaga accgaactca aggttactgt ggctgactcc tcactctctg gggtctactt |
| | 90601 | gcctgccacc ctcagagagc caaggatccg cctgtgatgc aggagtgagt gaagtcgctc |
| | 90661 | agccgagtcc gactctttgc aaccccatag gactgtagcc taccaggctc ctctgtctat |
| | 90721 | gggatttttc aggcaagagt gctggagtgg gttgccattt ccttctccag gggatcttcc |
| | 90781 | caaccctggt ctcccgcata gcaggcagac tctttactgt ctgagccacc aggcaatgca |
| | 90841 | ggagacctag gttcagtctc tgggtgggga agatcccctg gagaagggaa tgacaacctg |
| | 90901 | cttcagtatt cttgattggg gaatcccatg gacaaaggag cctggaggcc tacagcccat |
| | 90961 | agggtgcaaa gagacacgac tgagcaagtc acacacacag agccctacgt ggatgctcat |
| | 91021 | agcggcacct catagctgcc atgtatcagg tgttggcatg ggcagccatc agcagggggc |
| | 91081 | catttctgac ccactgcctt gttccaccgg atacacgggt gccttcctgt gtgtcgggcc |
| | 91141 | cactcggctg tcagcgccca agggcagggc tgtcgggagg cacagggcac agagttaagg |
| | 91201 | aggggatggg gacgttagct cctccccagc tctcagcgga tgcagcaggc aaaacaaacg |
| | 91261 | ctaggaatcc tgccaaaccc ggtagtctct gcccatgctc gccccatccc cagagccaca |
| | 91321 | agaacgggag ctggggggtg gcccggagct gggatactgg tccctgggcc cgcccatgtg |
| | 91381 | ctcggccgca cagcgtcctc cgggcgggga aactgaggca cgggcgcctc cggcttcctc |
| | 91441 | cccgccttcc gggcctcgcc tcgttcctcc tcaccagggc agtattccag ccccggctgt |
| | 91501 | gagacggaga agggcgccgt tcgagtcagg gccgcggctg ttatttctgc cggtgagcgg |
| | 91561 | ccttccctgg tacctccact tgagaggcgg ccgggaaggc cgagaaacgg gccgaggctc |
| | 91621 | ctttaagggg cccgtggggg cgcgcccggc ccttttgtcc gggtggcggc ggcggcgacg |
| | 91681 | cgcgcgtcag cgtcaacgcc cgcgcctgcg cactgagggc ggcctgcttg tcgtctgcgg |
| | 91741 | cggcggcggc ggcggcggcg gaggaggcga accccatctg gcttggcaag agactgagnn |
| | 91801 | nnnnnnnnnn nnnnnnnnnn nnnnnnnnnn nnnnnnnnnn nnnnnnnnnn nnnnnnnnnn |
| | 91861 | nnnnnnnnnn nnnnnnnnnn nnnnnnnnnn nnnnnnnnct gcaggtgccg gcggtgacgc |
| | 91921 | ggacgtacac cgcggcctgc gtcctcacca ccgccgccgt ggtaaccgcc cccgggggtt |
| | 91981 | gccaaggtta cgattggacc ctccccgccc cgaccctgct cccctagggt gggtgggtcg |
| | 92041 | gggggcagtt tctaagatct cctggttccg cagcagctgg aactcctcag tcccttccag |
| | 92101 | ctctacttca acccgcacct cgtgttccgg aagttccagg tgaggccgcc ccgccccttg |
| | 92161 | cacttgctgg cccaacccct cccgcccagc gctggcctga ccgcccccca ccccgcccac |
| | 92221 | cccacgcagg tttggaggct catcaccaac ttcctcttct tcgggcccct gggattcagc |
| | 92281 | ttcttcttca acatgctctt cgtgtatcct gcgccgtggt ggaagcggga ggagggcggg |
| | 92341 | gcgggggacc gggcgggagg cagcgggccc cgggaagctg agaccctcca aggggcacgc |
| | 92401 | ttcctatacc aaagccgcag gttccgctac tgccgcatgc tggaggaggg ctccttccgc |
| | 92461 | ggccgcacgg ccgacttcgt cttcatgttt ctcttcgggg gcgtcctgat gactgtatcc |
| | 92521 | ttcccgggct cggggaccta tgggtccggg cctctgctgg ccctgaggcc ctgcttgagc |
| | 92581 | gcatgccaca gagggagagt tgcgaccccg agctgagggt gtttttgagc gtacatcacg |
| | 92641 | tgctcagctg caggtgcccc tgtcgaactc cagggctaca cccaaaatac cacagggcag |
| | 92701 | ggtgcccagg ggctgagtcc tgaatgcagg tagccaggag gatctagggc tgggcccggg |
| | 92761 | ggctggggtg aagtggagag gcagggccga tcagggggcc cctggaggcc accgtttggt |
| | 92821 | cttagagtgg gaagcgaaac caacctgctt gagggtttca ggggtttagg aagtcagagg |
| | 92881 | ggccctgggc agggcacaag accttgactc tggcccagct actggggctc ctgggtagcc |
| | 92941 | tcttcttcct gggccaggcc ctcacggcca tgctggtgta cgtgtggagc cgccgcagcc |
| | 93001 | ctggggtgag ggtcaacttc tttggcctcc tcaccttcca ggcgccgttc ctgccctggg |
| | 93061 | cgctcatggg cttttcaatg ctgctgggca actccatcct ggtggacctg ctgggtgagc |
| | 93121 | ctgctgtcca gggagcctgc cccaagctgg gtgtgctggg ccagagccct ggtcctctcc |
| | 93181 | ccgcccccac ccctcttccc cactcctggc gcccccatcc ttccagcccc tccaacaagt |
| | 93241 | cagcctatag gttttactta ttcgagcctg acccatttgc tgacgcttgt gtggggcccg |
| | 93301 | acccggtagg gatgggtggc tcagggtgcc tgctcacagc tccacttctt ctgacgtcct |
| | 93361 | caggcctgac ctcctcccag gttctgccta ctctgggcca agcctggccc cacgctgggc |
| | 93421 | tggctggccg tgcagggcat cagaccccca tgctttgggg gcttcagggc tgtggagggt |
| | 93481 | ggcctcggca ttggcgcctc tcccacaggg attgcggtgg gccacgtcta ctacttcctg |
| | 93541 | gaggacgtct tccccaacca gcctggaggc aagaggctgc tgctgacccc cagcttcctg |
| | 93601 | tgagtgctga cagccttccc cacccccttc cccagatggc tctctacccc atgagggggg |
| | 93661 | gggaccctgc cagctgccgc tcagcgtggg ctcctcccca caggaaactg ctactggatg |
| | 93721 | ccccagagga ggaccccaat tacctgcccc tccccgagga gcagccagga cccctgcagc |
| | 93781 | agtgaggacg acctcaccca gagccgggtc ccccaccccc acccctggcc tgcaacgcag |
| | 93841 | ctccctgtcc tggaggccgg gcctgggccc agggcccccg ccctgaataa acaagtgacc |
| | 93901 | tgcagcctgt tcgccacagc actggctctc ctgccgcggc cagcctctcc acgcggggca |
| | 93961 | ggtgctgctg gccgagagcc agggccacca agcctgacgt gctctccgac ccagaacatt |
| | 94021 | ggcacagctg gaggcccaga gagggtccag aacctgccca ctcgccagca gaactctgag |
| | 94081 | cacagagggc agccctgctg gggttctcat ccctgccctg cctgtgccgt aattcagctt |
| | 94141 | ccactgatgg ggctcacatc tcaggggcgg ggctgggact gggatgctgg gttgtgctga |
| | 94201 | gctttggccg tgggggccct cctgtcccga actagcaacc cccaagggga cctctgcttc |
| | 94261 | atttcccagc caggccactg aaggacgggc caggtgcaga agagggccag gccctttctg |
| | 94321 | tgactccgaa gcctcaagtg tcagtgtttg cagagtccag tggctgaggc agaggcctct |
| | 94381 | gggaagctct gcccctgccg tttgcagctg aggccggcag gagcctcacc tggtccccag |
| | 94441 | ctcacgggca ttggaggacc agtccgcacg gtggtttact cctgggtcgg caccagccgc |
| | 94501 | cgccggctgt ccctttcaca gaggataaaa gtactcgctc tggagttgga ctttaatgtt |
| | 94561 | gtcatgaaac ctctggccca gcagcgggct ccgcagtggg tggcaggtga aggcccctcc |
| | 94621 | ccgggcctct ccaggcaggt gccgcctggc cagcagggaa ggcaggcagt gtcatccccc |
| | 94681 | actggctctg gggctcaggc tacctcctgc tgtggccgga acatctcccc cagtggtgga |
| | 94741 | gcccagtgtc cgtgaggcca gctgggcctg aaaccttcct ctctgaagcc ccgctgtccc |
| | 94801 | cttgccctgt atggagggca gaggctggag cgcaagttcc taggatgtgc ttgcgagacc |
| | 94861 | cccgagccca ggggcgaggc ccatctcagc ccacccccga actggaaacc cttggagctc |
| | 94921 | tgcccctcgt ggtgtgaggc ccctgctatg cgaccctcag ccctgccagc aacggaaggt |
| | 94981 | gcagggcccg ggcccacggg cttaacgcaa ctgggcctgg gtcacctgcg gggcctggtc |
| | 95041 | ccaggaggaa gacccaggtg ccaccctcct gggtgccacg tccaggtcac gtggggaccc |
| | 95101 | gtccatgtca cagaagatgc agggtcaccc ggtgagctgg cgccgggccc tgccagagca |
| | 95161 | ccagccgcgg gtggaggtgg gccccagctc tcctgtcagg cacgtggtgc tgggaggtgc |
| | 95221 | ggccggagca gtgcccacca gctgcagcag gacaggtggg cacaggccca ccagcagtgc |
| | 95281 | ccgcacggga tgggcccctg caagggccag agaagccacg ctcctggctg ggggctgggc |
| | 95341 | tgggactgac aggtggccct gccctctgcg ccccactact tcccagccac ccgggactcc |
| | 95401 | aaggacttgc tgagctgggc aggtgggacg ccgaggggag tcaaactgct cgtgggggca |
| | 95461 | ggaggggcgg tccacagggc tgagccctga gctgaaccct ggccctgctc gtggttgtgg |
| | 95521 | gggtgggggg gtccagtggc gccctagccc tgctgaggcc cagctgggac gtgcgcgccg |
| | 95581 | gagggcgagg ggccagccca tgccatgctg tcccccgttc tcagctccat gctaccactt |
| | 95641 | tgaagaaaca gaacctgttg cctttttatt tagaaagtgt tgcttgccct gcctggggct |
| | 95701 | tctatacaaa aaacaaacac agctcaacgt ggcctctcct gaccagagac gggcggtggg |
| | 95761 | gactggggct cagcagacgg aatgtgtccc cggcggcggg agaccaggag gcccctggcc |
| | 95821 | cgctcctcag gacggctggg ctgtccccac ctggtcccct ccgagccaga agatggagga |
| | 95881 | gaggtgggct gatctccaga tgctccctgg gagccaagcg ccacggggtg gtcaccaggc |
| | 95941 | cggggccgtg ttggccagac gcctcatccg cctgtgggag ggggagggca gcaacccccg |
| | 96001 | gatctctcag gcaaccgagt gaggaggcag gagcccccag cccctccctc ggccgctctg |
| | 96061 | ctgcgtgggg ccctgaagtc gtcctctgtc tcgcccccct ccccagggag agtgagcctg |
| | 96121 | ttctgggctg tggtcagacc tgcccgaggg ccagcctcgc ccggggccct gtcctgcctg |
| | 96181 | gaaggggctg gggcagcacc ttgtgttccg gtcctggtcc cggatcttct tctccatctc |
| | 96241 | tgcatccgtc agggtctcca gcagcgggca ccactggtca gcgtcgcctg tgttccggat |
| | 96301 | ggcaatctcc accgtgggca gggggttctc actgtggagg acgagagagg tagacggctc |
| | 96361 | acagagcagc tgcaggagag gcccctagaa agcagtgtcc accccgctgc gggcagacag |
| | 96421 | gacatggagc ctggtttctg cacccggctc ccgacacagg gcggccgggc acgctgccaa |
| | 96481 | catggcatct ccgggtctgc atgtggggag gggtccacag gacagtgctg caggtccagc |
| | 96541 | cattcccagt ggacttgctg ggaggaggag ggccgtccgc cccgctcagt gtccaggaga |
| | 96601 | aaggagagca aaggagtcca tccacccagg agtggagtcc cagggcccct gccctgacca |
| | 96661 | gcctgcaggg ggcccctcgg cccacatcac aggggcccag aatccataag ccctgactgc |
| | 96721 | tccaccccgg ggcccctcaa agacgcgcct agactccgtc cgagggccac ctgcacaccc |
| | 96781 | tctggcgaag tggactcagg gctgggggtc agcctcggtg aggccgcaaa ggctggggac |
| | 96841 | tcctggccga gctgctgcct ctgccaggag ccaggcccag cctgccggcg agcctcagcc |
| | 96901 | acgccctcac ccaccctgcc cgcggcgcca cgctggcctc cgggtcctct cctctggcct |
| | 96961 | cctgctgggc cactggtgct cagccccagc agtcggcctg ccaggagccc tgcagagtca |
| | 97021 | gcccccagag ggaggagggg gcccggggga acagcacagg aacaaacaga cccctggcct |
| | 97081 | tagttttagc tcctcatctg gaaaatgggg acagtgtcct tgctgcgagg ggtttcagag |
| | 97141 | gaccactgcc atgcaacacc cagcacacac ccactgcgtg ggggctcggg cccgagccgg |
| | 97201 | tgcccccgag tcccaggctg gtggctgggc cgccccagcc accctgccga cagctgcttc |
| | 97261 | ccagccgggc ggtgctgcgg cagtccagaa gccagcactg cagacccaaa tgtcactcct |
| | 97321 | cacgttgcgg gctcccagct gccttccttg ggggcagcag acacgaaagt caccaagccc |
| | 97381 | acgccgacgg gagcaaacac gtcttcctct taaacaagtg cgggtcccgg aggccctgtg |
| | 97441 | tttacctccc tgtggctccg ggaagattgc atcccagggg gttgttctaa accaagggct |
| | 97501 | gctcgggcca ggcctggaag gaggggcctg gagccaggag cccaccctta cgggcattcg |
| | 97561 | gcttcctggg tctcaaggcc ggctgggacc ctgcattccc accacccgcc aggtgcaagc |
| | 97621 | agggaggccg tgtcggagga ggcagagggc ctggagggtc gtcttcgacg tgacctcact |
| | 97681 | tttacaacct cacaggtgcg gcaggccagc tgggaggcat ggctgtgccc tcctggtaga |
| | 97741 | tgagaacaag actgcaggga gtgatccccc tgaacttccc caaccaggag gagacaaaac |
| | 97801 | tcggtgtcgc cctcctgctt aagatcaact gactctggac aaggggccca gcccacccga |
| | 97861 | tggggaaagg gcagtccttc caacaagcgg tgctgggacg ggacccggca ggccatggtt |
| | 97921 | tctcagctat gacaccagca gcacaagcac cccgagaaaa acagctaagc tgggcactgt |
| | 97981 | cacacaagtg aactccaaac ccaagaaaac cacaaaaagc ctgcggatct tcagatatgt |
| | 98041 | gggaagggac ctgtatctgg aatgtataac gaactcctga aaagtgaaag tgttagtcac |
| | 98101 | tcagtctgtt cagctctttg caaccccatg gacggtagcc tgccaggctc ctctgcccat |
| | 98161 | gggattctct aggcaagaat actggagtgg gttgccatgc cttcctccag gggatcttcc |
| | 98221 | caacccaggg attgaacctg tgtctctctt gcactggcag gcgggttctt taccagtagc |
| | 98281 | gccacctgag tagaaacact ccaggtgccc tgagtgtcag agcaggaggg actcggccca |
| | 98341 | ggcctgtgag gggaccctct ccgagtcccc tgctgcacag cagtgagagg tgcgttctga |
| | 98401 | gtcagcctcc agggatgagg gacttggtgt cgacatcact cccaggacct caggatctgc |
| | 98461 | tctgggaagc gaggctcccc aggctggccc caggcccgct ggcctcagct cgtgagccgt |
| | 98521 | gcgtggacag gtgccatgag caggcctccc acgggactcg gggcgcggcc tggaccccgg |
| | 98581 | ggctgccagt ggtcgcgggg ggccccgtgt ggcggctgtt ccctctcttg ctccgagtcc |
| | 98641 | taggaacatg gtgggcgctg cctcctgggg tttctggaga agcagctgag atgcaaacag |
| | 98701 | ccccacgcgc tccctcagct gttccctgtc acgggtggcc ccttggtgac ggcctccatg |
| | 98761 | cagggacggt gacagctcga gcagccgcgt aaaaccacac ggggacggtg gcagctcgag |
| | 98821 | cagccgcgta aagcctgaca tccaatttgg aagcctcccg cagtggaaga ggggcccggg |
| | 98881 | gacggggctg cccggggcga gctccaccgg gtcgggggtc acgaggagcc cacccgcgtc |
| | 98941 | cccgccacca gcacctggga ccagataccc tccccgctct gagggcggcc tgaacgccgc |
| | 99001 | cccctcccac gggggcgccc accgcctgct cgtggactga acaagaggcg gcagtggcct |
| | 99061 | ccagaccccc tcgggggagg gcagacctgt ccgagactga gcacaagtcc agggaatgag |
| | 99121 | caagggtctc agtaatgtcc ccaccgggac gggacgggag gaggcgacag aggccgctga |
| | 99181 | ggtgcggggc agccctcagt agctggcatc aaggccccag gcagtcccgg ggcatccccg |
| | 99241 | cagggggcgg gggcgaccac cggcccgagc ccaggcagtc ccggggcatc cctgcagcgg |
| | 99301 | gcgggggcga ccaccggccc gagccctacc tgaaggcgta ggtcttctga tgccagctca |
| | 99361 | gctgtccccg gatgctgtag gcgatggtgg tgacgaactc cccgcccagc cccagctcgg |
| | 99421 | agcacagctt cagagcgaac ttctcgggcg agttctcctt ctccgacatg tcccactcga |
| | 99481 | actggtccac caaggagatg ttccccacgt ggatgttcag ctggcccggg agcacagaca |
| | 99541 | tgagccagag cggccccctc tggggccagg ccgcaccctc accacccctt ctccccggaa |
| | 99601 | catccccgcc tcgttcttgg ccgcgcccct gtgctgctac ttggggtaag gaaaacaacc |
| | 99661 | cccatctctc tgaaaagggt taactagcga ggaagatgcg ctggtaactg gaaaactccc |
| | 99721 | tacaaagaaa gcttggatct gatggcttca ctggtgaatt ccaccaaaca tttcaagcac |
| | 99781 | taacaccaat ccttatcaaa tcctgccaaa aaactgaaaa ggaaggaaca catcataact |
| | 99841 | ccctgccttg ataccaaagc cagacaaaga tactacgaga aaggaaaggt gcagaccggc |
| | 99901 | acttactgtg gacattgatg tgaaacctca gcagacacga gcaaaactac attcaccagc |
| | 99961 | acgtcagaag aatcacacac cgttataaat gatgggatga tgacacaacc acattataaa |
| | 100021 | cggtggggct tactctggtg atgtaaggac ggctcagtaa gaaaaccggt caatgccatg |
| | 100081 | aaccacttga acagagtgaa ggacaaaaac cacacagtca tcttgataat tggaggaaaa |
| | 100141 | tcattagaca aacttcaacg tgctttcacg ataaaagcac tcagtaaact aagatcagat |
| | 100201 | ggaaaccaca tcaacaagat taattcagtc aaaaaattca ctgcaagtat cacccacaat |
| | 100261 | ggcagaagac tggtaacttt tcctctaaga tcaggaacga gccaaagata cccagtcttg |
| | 100321 | ccacttttgt tcaatatagc gttggaattt ctactcagtg cagtgcagtc gctcagtcgt |
| | 100381 | gtccgactct tttcgacccc atggatcaca gcacgccagg cctccctgtc catcaccaac |
| | 100441 | tcccggagtt cacccaaact catgtgcact gagtcagtga tgccatccag ccatctcatc |
| | 100501 | ctctgtcgtc cccttctcct cctgcctcca atcccttcca gcagttaggc aagaaaaata |
| | 100561 | aatcaaaggt atccacctgg aatggaagaa gtaaaactat ctctggtccg agatgttaca |
| | 100621 | atcttatatg cagagtttaa gatgctaaca aaatactatt agaactaatg aatgaattca |
| | 100681 | gcaaggtacc aggatacaaa gtcaacgtgc aaaaatcagc cgcatttcta catgctaaca |
| | 100741 | ctgcacaatc tgaagaagaa aggatgaaca aattacaata acataaaaaa gaataaaatc |
| | 100801 | cttagaaatt aacttgatca aagagatgta caatgaacaa tataaaacat actgaaagaa |
| | 100861 | attgaagata taaataaatg gaaaaacatc ctatgtccat ggattggaag acttaaaatt |
| | 100921 | attaagctgt caaggctatg gtttttccag tggtcatgta tggatgtgag agttggacta |
| | 100981 | taaagaaagc tgagcaccga agaagtgatg cttttgaact gtggtgttgg agaagactct |
| | 101041 | tgagaggtcc ttggactgca aggagatcca accagtccat cctaaaggag atcagtcctg |
| | 101101 | ggtgttcatt ggaaggactg atgttaaagc tgaaactcca atactttggc cacctgatgc |
| | 101161 | gaagagctga ctcatttgaa aagaccctga tgctgggtaa gattgagggc gggaggggaa |
| | 101221 | ggggacaaca gaggatgaga tggttggatg gcatcaccga ctcaatggac atgggtttgg |
| | 101281 | gtggactctg gaagttggtg atggacaggg aggcctggcg tgctgcggtt catggggttg |
| | 101341 | tgaggagtcg gacacgactg agcgactgaa ctgaactgaa catgaatacc caaagcaatc |
| | 101401 | tacaaagcca aatgtaatcc ctatcaaaat cccaatagca tttctgcaga aacaggaaaa |
| | 101461 | aaaatcttaa aattcatatg gaatctaagg aaaagcaaag gatgtctggt caaaacaatg |
| | 101521 | acgaaaagaa caacaaagct ggaagactca cacttcctga tttcagaact tactgcaaag |
| | 101581 | atacaataat gaaaacactg tgggactaac gtaaaagcag acacgtgggc caacgggaca |
| | 101641 | gcccagaaat aaactctcaa ataagcagtc aaatgatttt caacagagat gccaagacca |
| | 101701 | ctcagtgaag gaaagtgttt gcaaccaacg gttttgggaa aaaagaaccc acatgcgaaa |
| | 101761 | gaatgaagtg ggacccttac ccagccccat ctacagaaat caactcaaaa cagacagaac |
| | 101821 | atatggctca agccataaaa cgctcagaaa aacagagcaa agctttatga tgttggattt |
| | 101881 | ggcggtgatt tctcagatat gacgtcaaag gcataggtga taagcgaaaa aataaactgg |
| | 101941 | acttcaccaa aatacaacac ttctatgcat ccaaggacac taccgacagc ataacaaggc |
| | 102001 | agcccaggga aaggaggaaa catccgcaaa tcacagcatc tgggaacaga ccgctgcctg |
| | 102061 | tgagatacag ggaaccgata aaaacaagaa aacagcaaaa cccggactca aaaatgggaa |
| | 102121 | ggactccagc agacacagga gacagacaag ccgccagcag gtcactaatc agcaagcaag |
| | 102181 | gcccgcaaag gcccgtatcc aaggctgtgg tttttccagt ggtcatgtag gaaagagagc |
| | 102241 | tggatcgtaa gaaagctgag cgctgaagaa ttgattgaac tgtggtgttg gagaagactc |
| | 102301 | ttgagagtcc cttggactgc aagatcaaac cagtccattc tgaaggagat cagtcccgaa |
| | 102361 | tagtcactga aggactgatg ctgtagctcc aatactttgg ccacctgatt cgaagaactg |
| | 102421 | actcattggc aaagaccctg atgctgggaa agattgaagg caggaggaga aggggacgac |
| | 102481 | agaggatgag atggttggat ggcatcactg actccatgga catgagcttg ggcaagctcc |
| | 102541 | gggagagagt gaaggacagg gaagcctggc gtgctgcagc ccgtgggtcc caaatctttg |
| | 102601 | gaccaagcga ctgaacaata acaaatcaac agggaaatgc aaatcaaaac cacagtgaga |
| | 102661 | tactgtccac caccaggcag gcgttcttca gcggggttcg gggcaggtgg tgccctcttc |
| | 102721 | tctcgtaacg cccccaggac cgcgggggct gctgagacag catggggtgt gcttggccta |
| | 102781 | gcctgcccat gacaagagtg gcagtgtgct cgcctcactg cgcccttccc tgctctgccc |
| | 102841 | accagctggg ccacccctgg gaccacccag cttccgctcc gtggacggca aggccgcagc |
| | 102901 | agcgcccgga cacgcccaga acgtggtgcc ctcctcagaa gtcggcctgt gcccttcctg |
| | 102961 | ggacaagccg cccaagagac agtcttccag agccctgccc cacaacacgg accccagaca |
| | 103021 | ggctcctgtg gaggcctcca cgcacctccg cacctcgcaa gccccgagga caaggcaggc |
| | 103081 | ccgctgcggg tgaggagccg cctaccttga taatgacgcg ctggtctgac tggtcttcca |
| | 103141 | ggatgctgtc cgtggggtag gactcgatct gctgtctgat ggcagaggca atggctggca |
| | 103201 | cgaatgtcag tgggttcaga tccaggtcgt cacagagaat ctctgagaac atctccgggg |
| | 103261 | tcatcagctt ctctgaaacg atgacggagc gggggaaccc ccagtggacc acagggccta |
| | 103321 | cggtcagcgt gctcagcccc ggcctccccc agccttgcct cctctgccac cgcccccccg |
| | 103381 | ggtgacgaca ggaccccctg gcagcacgca gacagagctg agtgcacgcc agccagggcg |
| | 103441 | gcggacggac cattcatgtt ccaggtaaag gcatcccgca gcttctgccc gtcaatctcc |
| | 103501 | atgtccagtc ggatggggac cagcacctcg ggctgggacg cgttctcgtg gatcacggct |
| | 103561 | gggtcgtggt cgtcgaagct ggaaggggag cggccgcgtg ctcagcaaag cgggctgggc |
| | 103621 | ccctgtgccc agggcctccc tctctgcacc actggtcgct gagacctgcc cagagaggac |
| | 103681 | ctgtccacta cgggccgggc cggcagaaac agggctggcg ggggtccacg cggggcggga |
| | 103741 | ggggagctgc cgactcggca gcgggacaag ctcagaggtt ccctgcagga agagaggttt |
| | 103801 | aagccccaga gcaggcagga ttctcccagc agctgtgggg aagaaagggt atgtccagaa |
| | 103861 | gaagaaaccc tggaacaaag gccgaggggc aggagggttg aggagctgct tggagagcag |
| | 103921 | tgaagggggg ctgggcggct ggggggtgct ggggagcctc ggtggccaag cacccagggc |
| | 103981 | tccccacctg cagcctggac cccgagggag ccccagagga cggagagcaa ggcagctccg |
| | 104041 | cactcacacc tgccctttag gatggggaag agggaagaga cgggggctgc ggggggcaag |
| | 104101 | gaaaccaggc acgccccgct tagacccggg ggcgagaacc actttccaag aacgcagggg |
| | 104161 | cgccaatgat gaacaatggg tagcagcccg caggcgggag gcccggtggc cgaggcccct |
| | 104221 | caccagagcg ggaaggtccg cttcttgtcg cggcccatgc ggttcctgtt gatggtggtg |
| | 104281 | gagcagggca cggcgtccag gtggtgcgag ctgttgggca gggtgggcac ccactggctg |
| | 104341 | ttcctcttgg ccttctgttc cctgggagac acagacgccc gtccgctcag cctatgggcc |
| | 104401 | aaaagccgcc ccccagccgc caggttgtgg ccagtggacg cccgccatgc ccctctgggc |
| | 104461 | ccaggccccc atggggacct ctgtgcgccc agctccgcgg tggttattcc ccaggctcca |
| | 104521 | agcggcacct gctcggggtc accagtttta ggggaggagg agagggcagg ggccccagcc |
| | 104581 | cagtctgtga gctgtcaccc ccaggctcca agcggcacct gctcggggtc accagtttta |
| | 104641 | ggggaggagg agagggcagg ggccccagcc cagtctgtga gctgtcaccc ccaggctcca |
| | 104701 | agcggcacct gctcggggtc accagtttta ggggaggagg agagggcagg ggccccagcc |
| | 104761 | cagtctgtga gctgtcaccc ccaggctcca agcggcacct gctcggggtc accagtttta |
| | 104821 | ggggaggagg agagggcagg ggccccagcc cagtctgtga gctgtcaccc gtgctatgtg |
| | 104881 | ctgggctggg cactcaggaa agagggtcag ggttcacggg ggggtggcgc gcagatttcc |
| | 104941 | aggagagccc cgagggcagc agagaggagg ctcaggtcaa tggttgggca gggggccagg |
| | 105001 | gctggagaca cagagagggt cccgattcgg gggggtgccc tcagcaggtg gctgggagtc |
| | 105061 | cctgggggtt tgcacacttt cgatcaggct gttatttcag acgcttggtc cagcctgaga |
| | 105121 | caggtaatgc ctctggcctc cgggccttca gggatggaaa gatactctag aaagcgggac |
| | 105181 | tcaaagtaac tcaaggaact cgcgtcccac agtggggagc ccttctctcc aatttacatg |
| | 105241 | gggcgtttac tacgaggaaa ataccgaagg ccgttttgag ctgaggctcc cgggccgggc |
| | 105301 | tgtccgtttg tgagactgct cgtcacccct gggccacatc cctggtggcc aagggggcaa |
| | 105361 | tcagtgcggt gactgcacga cacacctctg cagccctgcc ccacagctgt caccatcggt |
| | 105421 | gacgtccacc ccctggagaa cctgaccact gcccggtttc ccgctaaaac agcgcccttc |
| | 105481 | caggatgggg ggcagaggga gaggccttgg ccttttcact cctcttctgc agcgggggcc |
| | 105541 | cctcgcaccc cagtgcccgg gcccaggagc gccccttggg gtggggcagg gagggatcca |
| | 105601 | cacaccaagg ggagccagga cccccccaaa tctgctgccc tgccctgata cccgagacct |
| | 105661 | ggggaaacgg gggactgggg ctgatgcggg caggaccaag aactgaggcg gtgagacggg |
| | 105721 | gtccccacca caggccatct ggctggcagt ttctactccg ggcctgcagg ccaagaggga |
| | 105781 | aaaggtgccc cactcagatc aggcgcctcc cgtccccagg gagggcctac aaggtcagat |
| | 105841 | cctttgtaac ttccacgggc aaaactggct tgctgggcct gtgcgggccg catgggcgtg |
| | 105901 | gaccaccaca cctttcccca ctgagtctcc agccggagct gtcacccagg tccccccagg |
| | 105961 | ccagccccac cccgccacct tgcagtagcc tctcgtatcc aggccgaggc tgcccggtcg |
| | 106021 | acccctcctg cctgatggcc tcaagtggac aatgcgagtc acgttgcagc acgtgagtgg |
| | 106081 | gacgggcagc gccacgcggg gtccgggcat ccgagtccca ccactcagcc tcccttccgc |
| | 106141 | tgcagagagg tctgtccaag agccctgggg gccatccagc ccctgtccga cctggccggt |
| | 106201 | gtggaagagg gggtgtgcca cccctcctgg ggggctggct gggcgctggg caggcccctc |
| | 106261 | ctaagagtgg agcccactgg tggttttcct gcagccccac ctccacacag cagttctcac |
| | 106321 | tgcccagtaa caggaggcta ctggcctagc tctctccctc gtgtgatgga ctcaaccagg |
| | 106381 | agcgttcacg gccccacaca gggttctcgg ctgctgcatg aggatctcaa agccccatcc |
| | 106441 | acgtgcatgt aatctcctcc ggtaacttct ctagggaagc ccggctatcc tgccatcctc |
| | 106501 | accgcaccac cagggcgaga aaagccatct ccagcgctca catccacaat gggccaggcc |
| | 106561 | gtgagcacac caccttcttc gggaggttgt gggggcgggn nnnnnnnnnn nnnnnnnnnn |
| | 106621 | nnnnnnnnnn nnnnnnnnnn nnnnnnnnnn nnnnnnnnnn nnnnnnnnnn nnnnnnnnnn |
| | 106681 | nnnnnnnnnn nnnnnnnnng cgcgcccccc ccccccgcgg cgccggcacc ccgggcggcg |
| | 106741 | gcccccggcg ctgggagcag gtgcggggcc gcggccgctc gtgagcctcc agcccggagg |
| | 106801 | acgggccccg ggggccggcc cggtgcccag gccctgggag ccccggaggc cagagtgcca |
| | 106861 | gagggccgga ggacccggga aggcccgaga gaggtgggaa gcacggggtt ccagccctag |
| | 106921 | gccatttcag ccccaaagcc atcggtgaaa ccattgctgg ccccagataa aagcgtcgcc |
| | 106981 | aactttttca ccccggcgga gactttagcg ggtagctgcc ccctaggggg aatggaaaaa |
| | 107041 | ccaggattta ccaggtgggt ggaggtcaca actgcccaga tcctgagaaa gaggggtcag |
| | 107101 | tggggcggga agattagtgg ggagaggagc tttcagaacc caagggaatg aaacgaggct |
| | 107161 | tgaggttggt tatccagcag ccgccccctg ccccgtgagt gagcgaaggc tgggcccctt |
| | 107221 | attgtcacat cttccagctc ttcgctagaa aacctagagt tttaaatact gtggcagctg |
| | 107281 | agtcaaacaa taaggaaaag cccgactctt tgagagccag gcacaaggcg tctgtgacag |
| | 107341 | ggtctccagg ctgcccattt gcagtctctg aaacggaggg tttttcgaga aggaggtctt |
| | 107401 | ggggtgcctg ccagaattgg aggggggggc gcgggaagtg aggacccaga agagagggct |
| | 107461 | tggcccgctg caaggaggtc actggacact ggagctgaag cgccagccga aactggaaac |
| | 107521 | tcgaaatctg tctccgtgcc agccacaagg cctatgattt tccttggcga cgttcagcat |
| | 107581 | cttaggagga gctggcgggg gaggcgggta gttcgtgggc ggttgcagca gggcaggaag |
| | 107641 | gtgaggaacc tgaggctggt cagagagctg gttggagtga tgcccatcgg tggacccgct |
| | 107701 | ggagaaggcc tgagtagaga aggtctaagc ttaacgggga aggggtgggc cagggtggaa |
| | 107761 | atggggtggg aagtttgagg agggggagca gtggagatgg gggttgtgag gaatgggagt |
| | 107821 | gagcttagac gtcttgagga tactgcagtt ctgtgctttt tttcacacct ggctgaaaat |
| | 107881 | tcactgaaaa caaaacaacc cttgctctgt gacagcctag aggggtggga gggaggctta |
| | 107941 | agagggaggg gacgtgcgtg tgcctatggg cgattcatgt gggtgtacgg cagaaagcaa |
| | 108001 | cacagtatgt aattaccctc caattaaaga tcaagtacaa cttaaaaacc ccaaacacaa |
| | 108061 | cattgtaagt cagctagact ccagtaaaca tttcagtaag aagattcaac tgggaatgag |
| | 108121 | ttccgccgtg actatcctga tgaatttccc gtgtcttctt gaggccattc ctctttgaac |
| | 108181 | ttccgtgttt ggggaagcgt gcctrtgtat ggagtcctga ggagtaaatg agacgggctt |
| | 108241 | gtagaaggcc tagtagtgcc ttgcacgcgg cagatgctca ataacctcga gttgtcacca |
| | 108301 | ttatggtacc tcaagagtct ccttggagct tgcacggttt ctgaatgggg tcctgcgggg |
| | 108361 | ctcccttggg gctcccacat ggggttgggg ggctgagtgg ggtgtccccg ctccttgctt |
| | 108421 | gtcccctgtg gaacaccccc ttccacccga gcagctctgc ttttgtctct tgtgtttgtt |
| | 108481 | tatatctcct agattgttgt tcagtcgctc agtcgtgtcc aactctccga ccccatggac |
| | 108541 | tgcagcacac caggccttct gccttcacca tctcccggag cttgctcaaa ctcctgtcca |
| | 108601 | ttgagttgct gatgccgtcc aaccatctcg tcctctgtcg tccccttctc cttttgacct |
| | 108661 | cagtctttcc cagcatcagg gtcttttcca atgagtcagc tctttgactc aggtggccaa |
| | 108721 | gtattggagc ttcagcttca ttatcagtcc ttccaatgaa tattcagggt tgatttcttt |
| | 108781 | taggattgag tgacttgatc tccttgcagt ccaagggact ctcaagagtc ttcaacacca |
| | 108841 | cagttcaaaa gcatcagttc ttcggcactc agccttcttt atgatccaac gcccacatcg |
| | 108901 | gtacatgact actggaaaaa ctttggctca gagataattg acttgattga atacaaagtt |
| | 108961 | ctttggcaaa aaataaaagt gtggcaagca gtactgacac aaaagcaagt ggcttttcct |
| | 109021 | ccgttgagtc atttatttat tcagtgggtg tgtgcgtgta gagacggagc ggctgtgctg |
| | 109081 | ggagctgggg cttccacttc agaggagccc cggacctgcc ctcggggagt tcacaggcag |
| | 109141 | tgctgcgggg ggtcctgcca ggacgcctgc cctgcgagtg cccagtgctg tgatggatgc |
| | 109201 | gtgtcccgca tctgcggcca ctggggccac gtgcccgaga ttgtccgggt ctgagggtgc |
| | 109261 | agagaagagg aggcatttgg actgagtctg gaaaaatgag catgtggcca cgtgagaagc |
| | 109321 | cagtggtgag gggaccagtc aggcggagga aagagcggct catacgagtt gtggagctgg |
| | 109381 | aagcatgagg gtgtgtggaa gcagaggccg gggacagggc cgcagggccg gccatggagg |
| | 109441 | gcgtgggctg ctgcaggctc ctgagaaggg ggacgctgcc atcatgaccg ggtttaggtg |
| | 109501 | tttgaccctg gtgtccacgt agaggacaga tgtgtggggg gggagctgga gatgggcatc |
| | 109561 | catcgggagt cagcctggag agaggcagag accccgtcag tgggccctca ggacgtggat |
| | 109621 | ggggcggatg ttgggaagat ctgactcctg ggttccggct ggggctccgg gctggagggg |
| | 109681 | tgccgcccac cgagcacagg aggcaaacag atgccctctc ccagcaagac cccagcccca |
| | 109741 | gcaccctccg gggccggact ccgcccctct tccagaatgg ctcccttgct gtcctcgccc |
| | 109801 | atctttccgg tgccctgagc ctctagagtc tggacaccag cgtccgcctt gcgcttgttt |
| | 109861 | ctgggaagtc tctggcttgt ctctgactca cccaggaccg tcttcgaggg caaggttgtg |
| | 109921 | tccttggttc catctgcttt ggggtccggc tcctcgctgc ttgacctgct gatgtgacag |
| | 109981 | tgtctcttgt tttcttttca gaatccgaga gcagctgtgt gtgtcccaga cagacccagc |
| | 110041 | cgctgggatg acgggcccct ctgtggagat ccccccggcc gccaagctgg gtgaggcttt |
| | 110101 | cgtgtttgcc ggcgggctgg acatgcaggc agacctgttc gcggaggagg acctgggggc |
| | 110161 | cccctttctt caggggaggg ctctggagca gatggccgtc atctacaagg agatccctct |
| | 110221 | cggggagcaa ggcagggagc aggacgatta ccggggggac ttcgatctgt gctccagccc |
| | 110281 | tgttccgcct cagagcgtcc ccccgggaga cagggcccag gacgatgagc tgttcggccc |
| | 110341 | gaccttcctc cagaaaccag acccgactgc gtaccggatc acgggcagcg gggaagccgc |
| | 110401 | cgatccgcct gccagggagg cggtgggcag gggtgacttg gggctgcagg ggccgcccag |
| | 110461 | gaccgcgcag cccgccaagc cctacgcgtg tcgggagtgc ggcaaggcct tcagccagag |
| | 110521 | ctcgcacctg ctccggcacc tggtgattca caccggggag aagccgtatg agtgcggcga |
| | 110581 | gtgcggcaag gccttcagcc agagctcgca cctgctccgg caccaggcca tccacaccgg |
| | 110641 | ggagaagccg tacgagtgcg gcgagtgcgg caaggccttc cggcagagct cggccctggc |
| | 110701 | gcagcacgcg aagacgcaca gcgggaggcg gccgtacgtc tgccgcgagt gcggcaagga |
| | 110761 | cttcagccgc agctccagcc tgcgcaagca cgagcgcatc cacaccgggg agaagcccta |
| | 110821 | cgcgtgccag gagtgcggca aggccttcaa ccagagctcg ggcctgagcc agcaccgcaa |
| | 110881 | gatccactcg ctgcagaggc cgcacgcctg cgagctgtgc gggaaggcct tctgccaccg |
| | 110941 | ctcgcacctg ctgcggcacc agcgcgtcca cacgggcaag aagccgtacg cctgcgcgga |
| | 111001 | ctgcggcaag gccttcagcc agagctccaa cctcatcgag caccgcaaga cgcacacggg |
| | 111061 | cgagaggccc taccggtgcc acaagtgcgg caaggccttc agccagagct cggcgctcat |
| | 111121 | cgagcaccag cgcacccaca cgggcgagag gccttacgag tgcggccagt gcggcaaggc |
| | 111181 | cttccgccac agctcggcgc tcatccagca ccagcgcacg cacacgggcc gcaagcccta |
| | 111241 | cgtgtgcaac gagtgcggca aggccttccg ccaccgctcg gcgctcatcg agcactacaa |
| | 111301 | gacgcacacg cgcgagcggc cctacgagtg caaccgctgc ggcaaggcct tccggggcag |
| | 111361 | ctcgcacctc ctccgccacc agaaggtcca cgcggcggac aagctctagg gtccgcccgg |
| | 111421 | ggcgagggca cgccggccct ggcgcccccg gcccagcggg tggacctggg gggccagccg |
| | 111481 | gacggcggaa tcccggccgg ctcttctctg ccgtgacccc ggggggttgg ttttgccctc |
| | 111541 | cattcgcttt ttctaaagtg cagacgaata cacgtcagag ggacgaagtg gggttaagcc |
| | 111601 | cccgggagac gtccggcgag ctctaacgtc agacacttga agaagtgaag cggactcgca |
| | 111661 | gcccgtacag cccggggaag atgagtccaa agtcgagggt caccttggcc actgcagggt |
| | 111721 | cgctcggcgg tggggcggag cgggtgcagg agggctcctc ctgggcttgg ggtggcaggc |
| | 111781 | gaggaccccg cgcctctcag ccctcggcct gggttggctg agggcgggcc tggctgtagg |
| | 111841 | ccctccagcg gaggtggagg cgctgcccgg ctcagccagg cacaggaccc tgccacgagg |
| | 111901 | agtagccctc cgccagaccc ggcgtccagg ctggggcgcc tgcggggcct ccgttctgtg |
| | 111961 | gctgggcagc ctgcgccctg tccagggatg aaggggttcc ggtctgaagg gctgggttca |
| | 112021 | gggtccagct ctggcccctc ctgccttggt gtcctggagg aagccccaag gctccgtttc |
| | 112081 | cctctccagg aggtggggac gttgggaatg ccacattccc ctggggggtg tgtgtgtgtg |
| | 112141 | ttcaaggctc ccattcagac tgggactggg cactcacgag ctttggcaac tggcaactga |
| | 112201 | ggacggagac ccagggtgac accccacctc ctgctgcggc ccccccggca ggggagacac |
| | 112261 | aggcccgtct ggttcccaag atggcagggc ccctccccct ccagcttgtg ccctgggtgt |
| | 112321 | ggtgcctggg gctacagcga ccctttccgg ttccccgggc cagttcagct gggcatcctc |
| | 112381 | agggcggggc tctgagggtg ccatgtttcc agagctcctc ctcctcccac cagtagcagg |
| | 112441 | cgggcggcca gctcccaggc agccccctgg catcgcctag gtgcacacct gcccgctgtg |
| | 112501 | acccagcaag gcttgaaggt ggccatccca gttaagtccc ctgcccctgg cccaggaatg |
| | 112561 | ggctcgggca gggccgcatc tggctgcccc agaagcgtct gtccctggcc tctgggagtt |
| | 112621 | ggcggtggtc tctggtactg tccctcgcag ggccccttag cactgctcgg ggaggaggtg |
| | 112681 | ggctgaactg attttgaagt tttacatgtc tgcggccgca gtcctacgag cccgtcaggg |
| | 112741 | tcatgctggt tatttcagca gatggggctt ggctcggcag ctaggatggt cctgaataaa |
| | 112801 | aatgggaagg ccagagctgt tcctccatca gcaggcttgg cagctgggga cgttgaaagg |
| | 112861 | acaggtctgc tggtctgggg agaccagctc tgtgcagccc ctgctgtccg tgggggtact |
| | 112921 | aaaccagccc ctgtgtgcgc ccatctgagt ggcagcccgc ctggaggatc gcccatcact |
| | 112981 | tgtgagaatt gagagaatgc tgacaccccc gcttggtgca gggggacagg gccccctaag |
| | 113041 | atctacctcc ttgccccacc cccgggaccc cctcagcctt ggccaggact gtccttactg |
| | 113101 | ggcagggcag tcatccactt ccaacctttg ccgtctcctc cgcgcgctgt gctcccagcc |
| | 113161 | aaattgtttt atttttttcc aagcatcact ttgcacacgt caccactctc cttaaaacca |
| | 113221 | cccttccgga gtctcctgct cgtaaatcgc cggtttcagc caacctgggt cgccccccaa |
| | 113281 | gcccagcaag cctgctgagc cccgcgcctc ccagctactt cacgctcgcc tcaagcttct |
| | 113341 | aaacgcggac cttctccccc ccacccccat ccctttcttt tctgatttat gtaacacggc |
| | 113401 | aggtaagact cctctcctga agggttgaca gactcacaca aaaccgtggt cagaccagge |
| | 113461 | aagtgctttt tttcagaagt gtgagcggaa cctagtcttc agctcatgct ctttccttgt |
| | 113521 | tttcttatgt gttctaagtc ctttgacttg ggctcccaga cagcgacgtt gtaagaggcc |
| | 113581 | gtcctggtag catttgaatt gtcctcgagt ttcgttgtcg gattttgttt tattgtctta |
| | 113641 | gttttccctt cttttagcag acgttgttga ctgtcgtaaa gctccagttc ttggttctgt |
| | 113701 | ttactaatca aattgttttg tcaaagtaca tgtattctgc tcttttcttt atcttttttg |
| | 113761 | ttgcttaata ttaacacttt acatttctaa gattaattat ttaggtaatt aataattttt |
| | 113821 | aacatttcta gtaaacgtgg gtacttgggt ctgtgtttgt tttcttgtag ttacagcttt |
| | 113881 | ttctgctcta tactgttgac gtctgggttt ttttttgctc ttaggaattt ccctttgacc |
| | 113941 | ccattattat tattttaatt agtatttttt aataattaaa aattagtgtt tttaaattaa |
| | 114001 | ccctaatcct aaccccagtg atgactgctt cagtcattgc tgttacttat tatgtgctgg |
| | 114061 | tgtcaggatt tttaagtgtc catagacatt ctctgagcct gaatatatta tcagttttat |
| | 114121 | acagcatttg tgtactctca agaaacgtgt tttcactctg tcagttcggt ttgttacctc |
| | 114181 | agtctttatg ttattttgct ccagtccgca cttgctctaa cttgtcttcc cttcgaggtg |
| | 114241 | tgaggacgcc tggcagccgg tgagcatgcc ggggtccggg gtcgtgggcc caggcgccca |
| | 114301 | gcaaagccct gtgggtgtgt gcacggctgg gctgctccgg gaggaagcct gtggccccac |
| | 114361 | ggtagttagg agcgctggtt tacctggtca caccacggtc tggttttgtg tgcttttccc |
| | 114421 | tgacgtgttt ctgttttgcc ttggtttcta ttctgtttta tgagtgccgt ttacgctttg |
| | 114481 | ttagtcatgc cgttatctcg atagacaggg tgtacgtgat caagtgatta ccgtatttgg |
| | 114541 | agcagatgtc tatttaacag agatgaactg agaacctgtg cctttgcatg ccctctttgc |
| | 114601 | ctcttttaat gcttctagct tcaacttctc ttttccaaac attataatgg aaaccccttg |
| | 114661 | cttttttttt tttaatttgc atttgcatga gagtttattt agctcggcat tttattttta |
| | 114721 | aaatttgtgt atatattttt gctatatatc tgtaacttat aaacagcaaa ttattggatt |
| | 114781 | ttgctttctg attctttctg taattcttct tacataagaa gttctcctat gagtaacatt |
| | 114841 | gctgtttaga gtgaggcatg atttatttcc agcttagtat gtattgggtc ggttaacccc |
| | 114901 | caaaggtcat gctcatcccc gccccatctc tgtgagttat tgtccgagtg tggagcgccc |
| | 114961 | tgtctaggcc gacgagagac ccaccatcgg gcacacctgc ccctcctggt ctggtcagtg |
| | 115021 | ccgggctctg tcctgagtcc actcctgatg tcacaggctg gtgcttcagc gacctcggct |
| | 115081 | gtgacacgga gggtgtgatg gcactgccca gccccatggg gcttggagga ctaaaggatg |
| | 115141 | cacacctgcc tggcagactg agggcacagg tgtttctcac actgtcagcg ttttgaaata |
| | 115201 | ttcctttgat tttctaccct aactcccaaa ggccgttcaa cataagctag aatgctacgt |
| | 115261 | ggtgcttgat tacattttag aaaagtttca gcaaatacca cgagatgcag caaagaacta |
| | 115321 | gacctcacag atcaggccgc ctgcataagg gagcccacac agtcgtggga gacggggacc |
| | 115381 | ctctcccacg tcctgtctgt cccaggatgg tcccctcacc cgccccctct ctcccctcgc |
| | 115441 | cctcctgtgg tgggggccgg ccaccatcac agctgcagag cctcaagaag ggggtcgccc |
| | 115501 | tggccactcc cgtggcagga gggacacgag ggcaggagct taccgcgggt gcagtggtct |
| | 115561 | cggatcagct cagctggccg ctgcggggtc ggggggacag ttcagtggga ggcaggagcc |
| | 115621 | cccactacag ctgccaggac ttctcagagg tgacaagggg gttcagtcac ctcagcccag |
| | 115681 | gtggaaacca aatggcctct tgcgcggctc ctggggccac gcggaggttc gctgggatca |
| | 115741 | caggtatctg gatgtgtgcg ccatggacat gcaccacctt cggggggtaa ggggtgggga |
| | 115801 | aaggcagccc ctttcttttg ggggaccccc tcttcagtgt ctgataacca ggaaaccaaa |
| | 115861 | tcagaaggtg gtctgggggt gctgagcagg gtgtctccta caccacaggc cacacactca |
| | 115921 | cacagcctcc aggactccag tggggctgag cgctggagac tcacccacgt ttgctacccc |
| | 115981 | cccacccaag gccatcccag aacagctgcc tgcgtcctca cggctggccc ctcccctctg |
| | 116041 | gtctaaccca gtgtgggtgg gccggcctgg ggtctccacc tgcctcctgc tgttccctgg |
| | 116101 | gctgctggct gtctgcagat gcggggccct ggcccggaga agccccatca gagcccagag |
| | 116161 | gacgggagtg gagcggggag gtgagccccg gagtctcgag gggccagagg caaaatactg |
| | 116221 | ggctgtgtcc ctggaaggca gtttcccatg aaaccttcaa tataggccgc cccagacgat |
| | 116281 | cagcctcatc tgctacgtgg attcctcccc gtagcgaatg gtgattgggt tctacatgga |
| | 116341 | cccgggactt ctgtttgaat tataatcttt cccccactgc ccctccaggg atctggaaaa |
| | 116401 | tggaggcctg ggctagacgg aagcttcctc caagattctt tattgaaggg attcgaagag |
| | 116461 | aaacaggtgg tcagtaatct gtgggggatg gaggggtgag cgctacgtgt aacggtttta |
| | 116521 | ctgttgctac gggaccagtt ttgatgtctt tccccttcaa gaagcagacc caaacaccga |
| | 116581 | gatgctgagg ttagcagcac agagcgggtt catccacaag gcaaccaggc agggagacca |
| | 116641 | gagacgctct ggaatctgcc tccctatggg cacgggctgg gtgctcacgg atgaagacca |
| | 116701 | agcagcaggt ggcgtggggc gtggggagcc tgcggaaagc gatggacaag gtgcgggacc |
| | 116761 | gcggtccgcg cggtggaccc aagctccgcc tctgcgctgc agcgcgagct gggggcggag |
| | 116821 | cttccaggga cccgcgaccg cgcccagtgg gagggtccgc ggtccaccca gtcctaacag |
| | 116881 | ctcagctcca gctagacgcc gctgagtccg gctttctaga gagcaacccc ggcgggtatt |
| | 116941 | ttatggttct ggcttcctga ttggaggaca cgcgagtctt agaacaccct tgattagtgc |
| | 117001 | gggcaggcgg aatggatttg actgatcacg atctgcagtt tcaccatctc aggggccgcc |
| | 117061 | ctcaccccca cctatcctgc caaagggggg gcctcggtgc tgagatcggg gccacacgtg |
| | 117121 | cactagacgg tcggtcagcg ctgctgctga gcggacccgg ggccatcctc acaccgccac |
| | 117181 | tggcccctgt gctcaataaa aggaaggaaa gcgggaaaag cgctttctgg ccgcggtggc |
| | 117241 | ctcgcgcgtt cctccatcgc catctgctgg cagagcccgg catggcaccc gctgcacaga |
| | 117301 | aacctcggtg tccgtttggg tgccccatcc ttgaccccga gagagcaccc tccgtccaaa |
| | 117361 | atgaaaaaca gctgctccca agagtcatta taatcacagc caattgtgtt aattcgtcct |
| | 117421 | cggatccact cacagttcca cggaacattc tgctaacctc tgacaactcc tacataaagc |
| | 117481 | aatactgaga agaaaagaac gtggttgata aatacaaagg catacaacaa taaggagcaa |
| | 117541 | agaaaaaaga cagtcctcgc agttctgttt tgttcatctc tcatgagtag gatggcagat |
| | 117601 | aaaacacaga atgcccagtg aataatttta gtctaagtat gtccccaata ctgcctaatc |
| | 117661 | ttcaaatcta accttatttt taaaatatat attttttgct ggtcactcat cagttcatgc |
| | 117721 | accaaagcct ttgtttcttg actcctaact ttttgacccc tctggggtga ggagcacccc |
| | 117781 | taacctcgag agcccatcac acagtcccct tgggactaga cccttctttg cccatcacag |
| | 117841 | ctgaccggaa gggccagccc atggccagcg ctcgcgcccc ctggcggaca gactctgcgc |
| | 117901 | ggcagccccg ggagcccagg tgcgaccccg cggtctctgg cgccctctag tgtggaaaga |
| | 117961 | tctcctcctg gtgttcccag tcattgggct gtattttatt agagaagatg ctcgcgtgac |
| | 118021 | gatgatgatg gtcctttacc gggaggcacg tttggggcgc gtcggctcag gggccgagct |
| | 118081 | attagcctgc atcgcgccca caggcatcgc gtccccctga gccgggtcag ctgtgggctg |
| | 118141 | tcctgacacg ggtttccccc agtctctggc ccgctgtccc tcccaggtca gtgtccagcg |
| | 118201 | ttgcccttct ggttgtggac ttgtgcagcg gtctcagcag atggaggggc gaccctaaag |
| | 118261 | gatgtattga ggcatctcag cactgtcctc cgcccaggtt tgctggtcag cagtgaagtg |
| | 118321 | accgggaaaa ggggctgtct tggggtcctt tcagaggcct gggttagacc aaagttttct |
| | 118381 | agaagattca ccattgcagg gagtcaaaga caaaactagg gtggtcagca atctgtgggg |
| | 118441 | gattcggcgg tgagggaatt ctgaatgcta catgtaatgg ttttactatt gttagggaac |
| | 118501 | atttttcccc cctacaaaca gcaggccaaa atactgagat gtcaggtttg catcaaagag |
| | 118561 | cgggttcatc cacaaggcaa ccagagaacg ctctggaatc tgcctccctg cgggcacagg |
| | 118621 | ctgggtgctc acggatgaag accaagcagc aggtggcgtg gggagtgggg agcctgggga |
| | 118681 | aagcgatgga caaggtgcga ggacctccgg cgcgagctgg aggcggagct tccagggaca |
| | 118741 | cgcggccacg cccagtggga gggtcagcgg tccatccagt cctaacagct cagctccaac |
| | 118801 | tagacgctgc tgagtctggc tttctagaga acactccggg cgggtatttt attgttttgg |
| | 118861 | cttcgtgact ggaggacgtt caagtcttaa aacacccttg attagtgcgg ggaggcggaa |
| | 118921 | tggatttgac tgatcacgac ccgcagtttc accatctcag gggccgccct caccccctcc |
| | 118981 | taccctacca aaggtggggg catcggtgct gagatctggg gtgacacata aaatcaggtg |
| | 119041 | aagtcttagg acagggggcc gattccaggt cctagggtgc agaaaaaacc tacctggccc |
| | 119101 | cgggctagac agcgtggagg gcgtggcccg ggctggtgca cagaagtggc ccccaactgg |
| | 119161 | tcagaaggtg tgggagccca gggctggtct actgcagaag gggtcgcctg gtggacagag |
| | 119221 | tggggcctga gtgcctgctg aactggtccg tcagggctgc tgagcagaca cgggccatca |
| | 119281 | tcactggctc ctgtgctcga tagaagggag ggaaaccagg aaagcaaagg cgctttatgg |
| | 119341 | ccgcttttgt gtttcgcgtt cctctagcac cgtctgccgg cagaacgcgg cattacatcc |
| | 119401 | gctggccaaa cctcggggtc cggcttggat gtccccatcc ttgtctcgga gatctcacct |
| | 119461 | ctcagcagtt cccctgggga caatgtcgag aagatgcgac cttgacccgg agctcggtgg |
| | 119521 | agagggtgcc ctgggttctt tccgcagttg cttggagtgg aggtgcctca tgttgggctg |
| | 119581 | ggaacgggag gaaggaaaca ggtcatgatt gagatgctct agacagactg tccctgctct |
| | 119641 | tgccaaattt cagaagattg tctttaataa atattccatt ttttgtatgc ccttaggtct |
| | 119701 | atttccagac actttaaata tattgaaaga ctttaaatat ttatataaaa atattattta |
| | 119761 | tagactgtat aaaaggaaca gttagaactg gacttggaac aacagactgg ttccaaatag |
| | 119821 | gaaaaggagt acgtcaaggc tgtatattgt caccctgctt atttaactta tatgcagagt |
| | 119881 | acatcatgag aaacgctggg ctggaagaaa cacaagctgg aatcaagatt gccgggagaa |
| | 119941 | atatcaataa cctcagatat gcagatgaca ccacccttat ggcagaaagt gaagaggaac |
| | 120001 | tcaaaagcct-cttgatgaag gtgaaagagg agagcgaaaa agttggctta aagctcaaca |
| | 120061 | tttagaaaac gaagatcatg gcatctggtc ccatcacttc atggaaatag atggggaaac |
| | 120121 | agttgagaca gtgtcagact ttatttttgg gggctccaat gaaattaaaa gacgcttact |
| | 120181 | tcttggaagg aaagttatga ccaacctaga cagcatatta aaaagcagag acactacttt |
| | 120241 | gccagcaaag gtccgtctag tcaaggctat ggtttttcca gtggtcatgt atggatgtga |
| | 120301 | gagttggact gtgaagaagg ctgagcaccg aagaagtgat gcttttgaac tgtggtgttg |
| | 120361 | gagaagactc ttgagaggcc cttggactgc aaggagatcc aaccagtcca tcgtaaagga |
| | 120421 | gatcaccccc tgggtggtca ttggaaggac tgatgttgaa gctgaaactc cagtactttg |
| | 120481 | gctacctaat gcgaagagct gactcattgg aaaagaccct gatgctggga aagattgaag |
| | 120541 | gtgggaggag aaggggacaa cagaggatga gatggttgga ttgcatcact gactcgatgg |
| | 120601 | acgtgagtct gagtgaagtc tgggagttgg tgatggccag ggaggccctg gcgtgctggc |
| | 120661 | ggttcatggg gtcgcaaaga gtcggccatg actgagtgac tgaactgaac tgatccagaa |
| | 120721 | atttaaaatt aatatataaa ccaaatccat gcagacaatt ataagcatat attataaatg |
| | 120781 | cataattata agcaagtata tgttatattt ataatagttt ataatgtatt tataagcaag |
| | 120841 | tatatattat tataagcata attgtaagta gaagtaactt tgggctttcc tggtggctca |
| | 120901 | gacagtaaag aatctgcctg cagtacagga gaccgggttc gatccctggt ttggggaaat |
| | 120961 | tccctggaga agggaatggc aaccaactcc aacatgtttg cctggagaat tccatggaca |
| | 121021 | gaggagcccg gaaggttgca gtccatgggg ttgcaaagag ctggatacaa cagagtgact |
| | 121081 | aacacatgta tataaataaa tttacctata tattgtatat atatttataa acatattcag |
| | 121141 | atattataaa taattagaaa catattatac atgtatttaa atactgttat aaacataaat |
| | 121201 | ttaaaaaata attttcagcc ctttggcttg ggggtgtgtt tgtggacgtc tttgtgctac |
| | 121261 | tgttcctgaa gtggagctct cccctcccaa accagctttt gaaatgactg ggaaagcaat |
| | 121321 | ggaatacata agcatcagga agatagcaac agagctgtca ttcttcacag agggtgtgct |
| | 121381 | tgagtgtgta gcaagtcccg cagaatgtag acagattaat atagtctatt aaaaatagtg |
| | 121441 | tagcaaattt acgaggtgcg atttcaagta taaagactta ctgggtctct cagttcagtt |
| | 121501 | cagtcgcttg gttgtgtccg actctttttg accccatgga ccgcagcacg ccaggcctcc |
| | 121561 | ctgtccatca ccaactcctg gagttcactc aaactcatgt ccatcgagtc ggtgatgcca |
| | 121621 | tccaaccatc tcatcctctg gcgtcccctt ctcctcccac cttcaatctt tcccagcatc |
| | 121681 | agggtctttc ccagtgagtc agttctttgc atcaggtggc cagagtagtg gagtttcagc |
| | 121741 | ttcagcatcg gtccttccaa tgaatattct ggactgattt cctttaggat tgactggttg |
| | 121801 | gatctccttg cagttcaagg gactctcaag agtcttctcc aacagcacag tctatgaata |
| | 121861 | gaatagcaaa tgaatagaga ataacattta cgaggatata ttttaccatt gcataaaata |
| | 121921 | tatcagcttg tagagaacag acttgttccc aggggagagg gtgggtaggg atggagtggg |
| | 121981 | agtttgngat cancagaagc gagctgttat atagaagatg gataaaaagg atacacaaca |
| | 122041 | atgtcctact gtgtggcacc gggacctata ttcagtagct tgtgagaaac cataatcgac |
| | 122101 | aagactgagg aaaagtatat atatatgtat gtacttgagt tgctttgctg tacagaagaa |
| | 122161 | attaacacaa cattgtaaat cgatatttca atagaatcca cccccccaaa tatataagtt |
| | 122221 | tcctggagat ggagacggca acccactcca tttcttgcac ccaatattct tgcctggagg |
| | 122281 | atcccatgga tagaggatcg caaagactcg gacataaccc agcgactaac actttccctt |
| | 122341 | tcaaatgtgt aggtttacta gcgtgaatct acagagatgc ccaagacatt cgtttatgag |
| | 122401 | gaaaactcca cacgcagctt cactgagaat tattaaacct attaaaggga gagagcgcca |
| | 122461 | ggatattcat ggattgaaag attcgatgtg gtcaagttgc cagttttccc caaactgatt |
| | 122521 | ggtaaattcc ccaggagctg gctcaaggcg caaaattccc tttacctttt tttaagagac |
| | 122581 | gaagccaagg agccgattct ggttgagaga cgctcaggtc ctcctgcggg agagcagccc |
| | 122641 | tcttcctccc ggtcgcctgg gcagtttcga ggccacgacc agaaggactt ggctccctgt |
| | 122701 | gtcgcgcact cagaagtctc cctctccgtc ccaaggactc agaagctggg cgtcctgccc |
| | 122761 | gcagcagagg aggcagcctg gaggggcccc gcgggcacag cggtccgggt ttcagccgag |
| | 122821 | ttgcccgccc cgcccctcta cctgggcgct gccgcccggc tccggggccg gccgtgccct |
| | 122881 | ccgtggccgc aaggcgtcgc tgtccccccg ctggaagtgc tgacccggag gaaggggccc |
| | 122941 | agacggaggg actcggagcc tccgagtgac accctgggac tccgagcgct ggagcctggc |
| | 123001 | gtcaccccag gcaggggcag tgggggcccg gggcggggtc aggggcctcc cccggttctc |
| | 123061 | atttgacacc gcgggggtgc gctgggcaca gtgtccaggg gccacgttcc gagcaggggc |
| | 123121 | gcgatgcagg cccgggcgcg gcctgtcccg ggcgcgagtc cagctgcttt gcagaggtgg |
| | 123181 | cggcaggtcg cagtgaccct cacagagacg ccccactctg cggctccagg tgggcctgtg |
| | 123241 | ccccccagaa gtgctgacct gtgcaccggg aaggcacagg gccccccagc catgtctgcg |
| | 123301 | atggaagagc cggaaccgcg ccatgcccgt cctcgctgac cggcaggcac ccgccgtgtg |
| | 123361 | tccacacgct gagccatctg gctccccttg cttgacatac acccaggacc tgagtgtgca |
| | 123421 | ggaagttaga aggggcaggt gtggtgacac gatgccatcc agcatcacct gagaacctgg |
| | 123481 | acaaacctca ggggcccagc ctgctctgtg aggccccgag ggccggcccc tccccggacc |
| | 123541 | cctgccttga atccggccac actgcccgcc ttcctgctcc tgcggcttgt cagacacgcc |
| | 123601 | tgagcccagg gcctgtgcac tcgctgtccc ttctgccagg actgctcctc cccaggctct |
| | 123661 | tgctggggct ccccttcttc attcgggggt ggcctctctt gttcagtggc tcagctgtgc |
| | 123721 | ccagtctttg caaccccatg gactgcagca cgccaggctt ccctgtcctt cactagctcc |
| | 123781 | tggagtttgc tcaaactcat gtccattgag tcagtgatgc tatccaacca tctcatcctt |
| | 123841 | tgctgcccac ttcttctcct gctctcaatc tttcccagca tcagggtctt ttccaatgag |
| | 123901 | ttagctctct gcatcaggag gccaaagtat tggagcttca gcatcagtcc ttccagtgaa |
| | 123961 | tatgcgaggt tgatttccct tagaattgac tggttggatc tccttcctgt ccagagaact |
| | 124021 | ctcaagagtc ttctccagca ccacagtcgg agagcatcag ttcttcagtg atcaggtttc |
| | 124081 | tttatagccc agctctcaca tcggtacatg actattggaa aacccatagc tttgattaga |
| | 124141 | tggaccttca ttggcaaagt gatgggcctt cattggccct gctttttaat acaccatcta |
| | 124201 | ggtttgtcgt agctttcctt ccaaagagca aacatctttt aatttcctgg ctgcagtaac |
| | 124261 | catccatagt gattttggag cccaagaaaa taaaatctgc cactgtttcc actttttccc |
| | 124321 | cttctatttg ctatgaagtg aggggactgg atgccatgat cttagtttaa accagcagtt |
| | 124381 | gtcaccccga ccgcttcctt tcctaaagag ctcatcacac ctcccactgg aatgcaatgt |
| | 124441 | gttgcctgtc cgcctgcttc acctcctggg actttgctgc aggtcttggt ctctgaggcc |
| | 124501 | cctgccgtat ccccagggcc cagagcagtg ctgggcttcg agtccgatca gggactatgt |
| | 124561 | gtgtggactg gatggtgctt gcttcttctg gggaacgaga gacctgggcc tggggaacga |
| | 124621 | ggggacctgg tgtgaccgga tctcctccct cgggagagga gccaagcgag tggacacagg |
| | 124681 | tcagtgtgtc ttgctcctgt gtggcaggtg tcccgtctgt gtctgtcatc ttggcatttc |
| | 124741 | ggtgtttctg tgaacccagc ccctcccctc ctgatacccc atcccatcag cacagaggag |
| | 124801 | actgggcttg gggactctct ggtcctgaga ttcctctccg catgtgactc ccccctcctg |
| | 124861 | gggggagcag gcaccgtgtg tgaggagggt ggaagctttt caagaccccc agcttttctg |
| | 124921 | tcccaggggg ctctggcagg gccttgggag ctggaatgag ctggaatctg ggccagtggg |
| | 124981 | ggtttccctg gtggtaaaga acccgcctgc ccatgcacga ggcataagag acgcgggttc |
| | 125041 | gatcactggg tcgggaagat cccctacagg agggcatggc aacccactcc agtattcttt |
| | 125101 | cctgaagaat cccttggaca gaggagcctg gtgggctaca gtctctgggg tggcaaggag |
| | 125161 | tcggacacga ctgaagcgac ttaccatgca cgcacgcggg gtcaggggtc agggccgcgc |
| | 125221 | tgcttacctg ctgtgtgacc ttagccaggt cacacccccc aggctgtgaa agagaacagt |
| | 125281 | cttcccagac tcgggcatcc aggtctttac agacgtgcct gtgagctttg tgactctggc |
| | 125341 | tctgtggccg ctagagggcg ctgtccgccg ggccctatgt gcgtgcacgc atgtgagcat |
| | 125401 | gttcgcatac gtgtgtgcat ctgtcggggg cgcacggtgc ggggacacgg gcacgcggtc |
| | 125461 | aggaacgcag cccggacacc tccacgtggc ccgcgagtac cgtcaggtgg gggctgtggc |
| | 125521 | tccgctgtgt gggtgacccg ccctcccccc gcgaacgtgg tgcatagtga ccgcctggct |
| | 125581 | gggctcctga gctcagccat cctgcccccc gggtcagctc ccgacaggcc cagctctagg |
| | 125641 | ccccaggcgt ggaccgaggc ccccaggccc cggcctgtga gatgggacct ccgtctgggg |
| | 125701 | ggctcattct gctcccggag gcctggcagg cccctcctct ttggcattgc ataccctcgc |
| | 125761 | attggggtgg gtaagcacag taccccatgc ctgtggcccc gtgggagcgg cctgctcagg |
| | 125821 | gaggccggag cctcagctac agggctgtca caccgggctg cagaggaaga agacgggagc |
| | 125881 | gaggcctaca ggaacctagc caggccctgg cccactgagc cgacaggagc ctggccagag |
| | 125941 | gcctgcacag gacggggtgg cggggggggt ggggtggggt gctgggcccc gtggccttga |
| | 126001 | ctgcagaccc cgagggctcc tcagcttaga acggccaagc ctgagtcttg ggggtgcagg |
| | 126061 | tcaggggg |

Primers

In another embodiment, primers are provided to generate 3′ and 5′ sequences of a targeting vector. The oligonucleotide primers can be capable of hybridizing to porcine immunoglobulin genomic sequence, such as Seq ID Nos. 1, 4, 29, 30, 12, 25, 15, 16, 19, 28 or 31, as described above. In a particular embodiment, the primers hybridize under stringent conditions to Seq ID Nos. 1, 4, 29, 30, 12, 25, 15, 16, 19, 28 or 31, as described above. Another embodiment provides oligonucleotide probes capable of hybridizing to porcine heavy chain, kappa light chain or lambda light chain nucleic acid sequences, such as Seq ID Nos. 1, 4, 29, 30, 12, 25, 15, 16, 19, 28 or 31, as described above. The polynucleotide primers or probes can have at least 14 bases, 20 bases, 30 bases, or 50 bases which hybridize to a polynucleotide of the present invention. The probe or primer can be at least 14 nucleotides in length, and in a particular embodiment, are at least 15, 20, 25, 28, or 30 nucleotides in length.

In one embodiment, primers are provided to amplify a fragment of porcine Ig heavy-chain that includes the functional joining region (the J6 region). In one non-limiting embodiment, the amplified fragment of heavy chain can be represented by Seq ID No 4 and the primers used to amplify this fragment can be complementary to a portion of the J-region, such as, but not limited to Seq ID No 2, to produce the 5′ recombination arm and complementary to a portion of Ig heavy-chain mu constant region, such as, but not limited to Seq ID No 3, to produce the 3′ recombination arm. In another embodiment, regions of the porcine Ig heavy chain (such as, but not limited to Seq ID No 4) can be subcloned and assembled into a targeting vector.

In other embodiments, primers are provided to amplify a fragment of porcine Ig kappa light-chain that includes the constant region. In another embodiment, primers are provided to amplify a fragment of porcine Ig kappa light-chain that includes the J region. In one non-limiting embodiment, the primers used to amplify this fragment can be complementary to a portion of the J-region, such as, but not limited to Seq ID No 21 or 10, to produce the 5′ recombination arm and complementary to genomic sequence 3′ of the constant region, such as, but not limited to Seq ID No 14, 24 or 18, to produce the 3′ recombination arm. In another embodiment, regions of the porcine Ig heavy chain (such as, but not limited to Seq ID No 20) can be subcloned and assembled into a targeting vector.

II. Genetic Targeting of the Immunoglobulin Genes

The present invention provides cells that have been genetically modified to inactivate immunoglobulin genes, for example, immunoglobulin genes described above. Animal cells that can be genetically modified can be obtained from a variety of different organs and tissues such as, but not limited to, skin, mesenchyme, lung, pancreas, heart, intestine, stomach, bladder, blood vessels, kidney, urethra, reproductive organs, and a disaggregated preparation of a whole or part of an embryo, fetus, or adult animal. In one embodiment of the invention, cells can be selected from the group consisting of, but not limited to, epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, granulosa cells, cumulus cells, epidermal cells, endothelial cells, Islets of Langerhans cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, and osteoclasts. In one alternative embodiment, embryonic stem cells can be used. An embryonic stem cell line can be employed or embryonic stem cells can be obtained freshly from a host, such as a porcine animal. The cells can be grown on an appropriate fibroblast-feeder layer or grown in the presence of leukemia inhibiting factor (LIF).

In a particular embodiment, the cells can be fibroblasts; in one specific embodiment, the cells can be fetal fibroblasts. Fibroblast cells are a suitable somatic cell type because they can be obtained from developing fetuses and adult animals in large quantities. These cells can be easily propagated in vitro with a rapid doubling time and can be clonally propagated for use in gene targeting procedures.

Targeting constructs

Homologous Recombination

In one embodiment, immunoglobulin genes can be genetically targeted in cells through homologous recombination. Homologous recombination permits site-specific modifications in endogenous genes and thus novel alterations can be engineered into the genome. In homologous recombination, the incoming DNA interacts with and integrates into a site in the genome that contains a substantially homologous DNA sequence. In non-homologous (“random” or “illicit”) integration, the incoming DNA is not found at a homologous sequence in the genome but integrates elsewhere, at one of a large number of potential locations. In general, studies with higher eukaryotic cells have revealed that the frequency of homologous recombination is far less than the frequency of random integration. The ratio of these frequencies has direct implications for “gene targeting” which depends on integration via homologous recombination (i.e. recombination between the exogenous “targeting DNA” and the corresponding “target DNA” in the genome).

A number of papers describe the use of homologous recombination in mammalian cells. Illustrative of these papers are Kucherlapati et al., Proc. Natl. Acad. Sci. USA 81:3153-3157, 1984; Kucherlapati et al., Mol. Cell. Bio. 5:714-720, 1985; Smithies et al, Nature 317:230-234, 1985; Wake et al., Mol. Cell. Bio. 8:2080-2089, 1985; Ayares et al., Genetics 111:375-388, 1985; Ayares et al., Mol. Cell. Bio. 7:1656-1662, 1986; Song et al., Proc. Natl. Acad. Sci. USA 84:6820-6824, 1987; Thomas et al. Cell 44:419-428, 1986; Thomas and Capecchi, Cell 51: 503-512, 1987; Nandi et al., Proc. Natl. Acad. Sci. USA 85:3845-3849, 1988; and Mansour et al., Nature 336:348-352, 1988. Evans and Kaufman, Nature 294:146-154, 1981; Doetschman et al., Nature 330:576-578, 1987; Thoma and Capecchi, Cell 51:503-512,4987; Thompson et al., Cell 56:316-321, 1989.

The present invention can use homologous recombination to inactivate an immunoglobulin gene in cells, such as the cells described above. The. DNA can comprise at least a portion of the gene(s) at the particular locus with introduction of an alteration into at least one, optionally both copies, of the native gene(s), so as to prevent expression of functional immunoglobulin. The alteration can be an insertion, deletion, replacement or combination thereof. When the alteration is introduce into only one copy of the gene being inactivated, the cells having a single unmutated copy of the target gene are amplified and can be subjected to a second targeting step, where the alteration can be the same or different from the first alteration, usually different, and where a deletion, or replacement is involved, can be overlapping at least a portion of the alteration originally introduced. In this second targeting step, a targeting vector with the same arms of homology, but containing a different mammalian selectable markers can be used. The resulting transformants are screened for the absence of a functional target antigen and the DNA of the cell can be further screened to ensure the absence of a wild-type target gene. Alternatively, homozygosity as to a phenotype can be achieved by breeding hosts heterozygous for the mutation.

Targeting Vectors

In another embodiment, nucleic acid targeting vector constructs are also provided. The targeting vectors can be designed to accomplish homologous recombination in cells. These targeting vectors can be transformed into mammalian cells to target the ungulate heavy chain, kappa light chain or lambda light chain genes via homologous recombination. In one embodiment, the targeting vectors can contain a 3′ recombination arm and a 5′ recombination arm (i.e. flanking sequence) that is homologous to the genomic sequence of ungulate heavy chain, kappa light chain or lambda light chain genomic sequence, for example, sequence represented by Seq ID Nos. 1, 4, 29, 30, 12, 25, 15, 16, 19, 28 or 31, as described above. The homologous DNA sequence can include at least 15 bp, 20 bp, 25 bp, 50 bp, 100 bp, 500 bp, 1 kbp, 2 kbp, 4 kbp, 5 kbp, 10 kbp, 15 kbp, 20 kbp, or 50 kbp of sequence, particularly contiguous sequence, homologous to the genomic sequence. The 3′ and 5′ recombination arms can be designed such that they flank the 3′ and 5′ ends of at least one functional variable, joining, diversity, and/or constant region of the genomic sequence. The targeting of a functional region can render it inactive, which results in the inability of the cell to produce functional immunoglobulin molecules. In another embodiment, the homologous DNA sequence can include one or more intron and/or exon sequences. In addition to the nucleic acid sequences, the expression vector can contain selectable marker sequences, such as, for example, enhanced Green Fluorescent Protein (eGFP) gene sequences, initiation and/or enhancer sequences, poly A-limiting tail sequences, and/or nucleic acid sequences that provide for the expression of the construct in prokaryotic and/or eukaryotic host cells. The selectable marker can be located between the 5′ and 3′ recombination arm sequence.

Modification of a targeted locus of a cell can be produced by introducing DNA into the cells, where the DNA has homology to the target locus and includes a marker gene, allowing for selection of cells comprising the integrated construct. The homologous DNA in the target vector will recombine with the chromosomal DNA at the target locus. The marker gene can be flanked on both sides by homologous DNA sequences, a 3′ recombination arm and a 5′ recombination arm. Methods for the construction of targeting vectors have been described in the art, see, for example, Dai et al., Nature Biotechnology 20: 251-255, 2002; WO 00/51424.

Various constructs can be prepared for homologous recombination at a target locus. The construct can include at least 50 bp, 100 bp, 500 bp, 1 kbp, 2 kbp, 4 kbp, 5 kbp, 10 kbp, 15 kbp, 20 kbp, or 50 kbp of sequence homologous with the target locus. The sequence can include any contiguous sequence of an immunoglobulin gene.

Various considerations can be involved in determining the extent of homology of target DNA sequences, such as, for example, the size of the target locus, availability of sequences, relative efficiency of double cross-over events at the target locus and the similarity of the target sequence with other sequences.

The targeting DNA can include a sequence in which DNA substantially isogenic flanks the desired sequence modifications with a corresponding target sequence in the genome to be modified. The substantially isogenic sequence can be at least about 95%, 97-98%, 99.0-99.5%, 99.6-99.9%, or 100% identical to the corresponding target sequence (except for the desired sequence modifications). In a particular embodiment, the targeting DNA and the target DNA can share stretches of DNA at least about 75, 150 or 500 base pairs that are 100% identical. Accordingly, targeting DNA can be derived from cells closely related to the cell line being targeted; or the targeting DNA can be derived from cells of the same cell line or animal as the cells being targeted.

Porcine Heavy Chain Targeting

In particular embodiments of the present invention, targeting vectors are provided to target the porcine heavy chain locus. In one particular embodiment, the targeting vector can contain 5′ and 3′ recombination arms that contain homologous sequence to the 3′ and 5′ flanking sequence of the J6 region of the porcine immunoglobulin heavy chain locus. Since the J6 region is the only functional joining region of the porcine immunoglobulin heavy chain locus, this will prevent the exression of a functional porcine heavy chain immunoglobulin. In a specific embodiment, the targeting vector can contain a 5′ recombination arm that contains sequence homologous to genomic sequence 5′ of the J6 region, optionally including J1-4 and a 3′ recombination arm that contains sequence homologous to genomic sequence 3′ of the J6 region, including the mu constant region (a “J6 targeting construct”), see for example, FIG. 1. Further, this J6 targeting construct can also contain a selectable marker gene that is located between the 5′ and 3′ recombination arms, see for example, Seq ID No 5 and FIG. 1. In other particular embodiments, the 5′ targeting arm can contain sequence 5′ of J1, such as depicted in Seq ID No. 1 and/or Seq ID No 4. In another embodiments, the 5′ targeting arm can contain sequence 5′ of J1, J2 and/or J3, for example, as depicted in approximately residues 1-300, 1-500, 1-750, 1-1000 and/or 1-1500 Seq ID No 4. In a further embodiment, the 5′ targeting arm can contain sequence 5′ of the constant region, for example, as depicted in approximately residues 1-300, 1-500, 1-750, 1-1000, 1-1500 and/or 1-2000 or any fragment thereof of Seq ID No 4 and/or any contiguous sequence of Seq ID No. 4 or fragment thereof. In another embodiment, the 3′ targeting arm can contain sequence 3′ of the constant region and/or including the constant region, for example, such as resides 7000-8000 and/or 8000-9000 or fragment thereof of Seq ID No 4. In other embodiments, targeting vector can contain any contiguous sequence or fragment thereof of Seq ID No 4. sequence In other embodiments, the targeting vector can contain a 5′ recombination arm that contains sequence homologous to genomic sequence 5′ of the diversity region, and a 3′ recombination arm that contains sequence homologous to genomic sequence 3′ of the diversity region of the porcine heavy chain locus. In a further embodiment, the targeting vector can contain a 5′ recombination arm that contains sequence homologous to genomic sequence 5′ of the mu constant region and a 3′ recombination arm that contains sequence homologous to genomic sequence 3′ of the mu constant region of the porcine heavy chain locus.

In further embodiments, the targeting vector can include, but is not limited to any of the following sequences: the Diversity region of heavy chain is represented, for example, by residues 1089-1099 of Seq ID No 29 (D(pseudo)), the Joining region of heavy chain is represented, for example, by residues 1887-3352 of Seq ID No 29 (for example: J(psuedo): 1887-1931 of Seq ID No 29, J(psuedo): 2364-2411 of Seq ID No 29, J(psuedo): 2756-2804 of Seq ID No 29, J (functional J): 3296-3352 of Seq ID No 29), the recombination signals are represented, for example, by residues 3001-3261 of Seq ID No 29 (Nonamer), 3292-3298 of Seq ID No 29 (Heptamer), the Constant Region is represented by the following residues: 3353-9070 of Seq ID No 29 (J to C mu intron), 5522-8700 of Seq ID No 29 (Switch region), 9071-9388 of Seq ID No 29 (Mu Exon 1), 9389-9469 of Seq ID No 29 (Mu Intron A), 9470-9802 of Seq ID No 29 (Mu Exon 2), 9830-10069 of Seq ID No 29 (Mu Intron B), 10070-10387 of Seq ID No 29 (Mu Exon 3), 10388-10517 of Seq ID No 29 (Mu Intron C), 10815-11052 of Seq ID No 29 (Mu Exon 4), 11034-11039 of Seq ID No 29 (Poly(A) signal) or any fragment or combination thereof. Still further, any contiguous sequence at least about 17, 20, 30, 40, 50, 100, 150, 200 or 300 nucleotides of Seq ID No 29 or fragment and/or combination thereof can be used as targeting sequence for the heavy chain targeting vector. It is understood that in general when designing a targeting construct one targeting arm will be 5′ of the other targeting arm.

In other embodiments, targeting vectors designed to disrupt the expression of porcine heavy chain genes can contain recombination arms, for example, the 3′ or 5′ recombination arm, that target the constant region of heavy chain. In one embodiment, the recombination arm can target the mu constant region, for example, the C mu sequences described above or as disclosed in Sun & Butler Immunogenetics (1997) 46: 452-460. In another embodiment, the recombination arm can target the delta constant region, such as the sequence disclosed in Zhao et al. (2003) J Immunol 171: 1312-1318, or the alpha constant region, such as the sequence disclosed in Brown & Butler (1994) Molec Immunol 31: 633-642.

| | |
|---|---|
| Seq ID No.5 | GGCCAGACTTCCTCGGAACAGCTCAAAGAGCTCTGTCAA |
| | AGCCAGATCCCATCACACGTGGGCACCAATAGGCCATGC |
| | CAGCCTCCAAGGGCCGAACTGGGTTCTCCACGGCGCACA |
| | TGAAGCCTGCAGCCTGGCTTATCCTCTTCCGTGGTGAAG |
| | AGGCAGGCCCGGGACTGGACGAGGGGCTAGCAGGGTGTG |
| | GTAGGCACCTTGCGCCCCCCACCCCGGCAGGAACCAGAG |
| | ACCCTGGGGCTGAGAGTGAGCCTCCAAACAGGATGCCCC |
| | ACCCTTCAGGCCACCTTTCAATCCAGCTACACTCCACCT |
| | GCCATTCTCCTCTGGGCACAGGGCCCAGCCCCTGGATCT |
| | TGGCCTTGGCTCGACTTGCACCCACGCGCACACACACAC |
| | TTCCTAACGTGCTGTCCGCTCACCCCTCCCCAGGGTGGT |
| | CCATGGGCAGCACGGCAGTGGGCGTCCGGCGGTAGTGAG |
| | TGCAGAGGTCCCTTCCCCTCCCCCAGGAGCCCCAGGGGT |
| | GTGTGCAGATCTGGGGGCTCCTGTCCCTTACACCTTCAT |
| | GCCCCTCCCCTCATACCCACCCTCCAGGCGGGAGGCAGC |
| | GAGACCTTTGCCCAGGGACTCAGCCAACGGGCACAGGGG |
| | AGGCCAGCCCTCAGCAGCTGGCTCCCAAAGAGGAGGTGG |
| | GAGGTAGGTCCACAGCTGCCACAGAGAGAAACCCTGACG |
| | GACCCCACAGGGGCCACGCCAGCCGGAACCAGCTCCCTC |
| | GTGGGTGAGCAATGGCCAGGGCCCGGCCGGCGACCACGG |
| | CTGGCGTTGCGCCAGGTGAGAACTCACGTCCAGTGCAGG |
| | GAGACTCAAGACAGCGTGTGCACACAGGGTCGGATCTGC |
| | TCCCATTTCAAGCAGAAAAAGGAAACCGTGCAGGCAGCC |
| | GTCAGCATTTCAAGGATTGTAGCAGCGGCCAACTATTCG |
| | TCGGCAGTGGCCGATTAGAATGACCGTGGAGAAGGGCGG |
| | AAGGGTCTCTCGTGGGCTCTGCGGCCAACAGGCCCTGGC |
| | TCCACCTGCCCGCTGCCAGGCCGAGGGGCTTGGGCCGAG |
| | CCAGGAACCACAGTGCTCACCGGGAGCACAGTGACTGAC |
| | CAAACTCCGGGCCAGAGCAGCGCCAGGCGAGCCGGGGTC |
| | TCGCCGTGGAGGACTCACCATCAGATGCACAAGGGGGCG |
| | AGTGTGGAAGAGACGTGTCGCCCGGGCCATTTGGGAAGG |
| | CGAAGGGACCTTCCAGGTGGACAGGAGGTGGGACGCACT |
| | CCAGGCAAGGGACTGGGTCCCCAAGGCCTGGGGAAGGGG |
| | TACTGGCTTGGGGGTTAGCCTGGCCAGGGAACGGGGAGC |
| | GGGGCGGGGGGGTGAGCAGGGAGGACCTGAGCTGGTGGG |
| | AGCGAGGCAAGTCAGGCTTCAGGCAGCAGCCGCAGATCC |
| | CAGACCAGGAGGGTGAGGCAGGAGGGGGTTGCAGGGGGG |
| | CGGGGGCCTGCCTGGCTCCGGGGGCTCCTGGGGGACGCT |
| | GGCTCTTGTTTCCGTGTCCCGCAGCACAGGGCCAGCTCG |
| | CTGGGCCTATGCTTACCTTGATGTCTGGGGCCGGGGCGT |
| | CAGGGTCGTCGTCTCCTCAGGGGAGAGTCCCCTGAGGCT |
| | ACGGTGGGG*GGGGACTATGGCAGCTCCACCAGGGGCCT |
| | GGGGACCAGGGGCCTGGACCAGGCTGCAGCCCGGAGGAC |
| | GGGGAGGGCTGTGGCTCTCCAGCATCTGGCCCTCGGAAA |
| | TGGCAGAACGGCTGGCGGGTGAGCGAGCTGAGAGCGGGT |
| | CAGACAGACAGGGGCCGGCCGGAAAGGAGAAGTTGGGGG |
| | CAGAGCCCGCCAGGGGCCAGGCCCAAGGTTCTGTGTGCC |
| | AGGGCCTGGGTGGGCACATTGGTGTGGCCATGGCTACTT |
| | AGACGCGTGATCAAGGGCGAATTCCAGCACACTGGCGGC |
| | CGTTACTAGTggatcccggcgcgccctaccgggtagggg |
| | aggcgcttttcccaaggcagtctggagcatgcgctttag |
| | cagccccgctgggcacttggcgctacacaagtggcctct |
| | ggcctcgcacacattccacatccaccggtaggcgccaac |
| | cggctccgttctttggtggccccttcgcgccaccttcta |
| | ctcctcccctagtcaggaagttcccccccgccccgcagc |
| | tcgcgtcgtgcaggacgtgacaaatggaagtagcacgtc |
| | tcactagtctcgtgcagatggacagcaccgctgagcaat |
| | ggaagcgggtaggcctttggggcagcggccaatagcagc |
| | tttggctccttcgctttctgggctcagaggctgggaagg |
| | ggtgggtccgggggcgggctcaggggcgggctcaggggc |
| | ggggcgggcgcccgaaggtcctccggaagcccggcattc |
| | tgcacgcttcaaaagcgcacgtctgccgcgctgttctcc |
| | tcttcctcatctccgggcctttcgacctgcagccaatat |
| | gggatcggccattgaacaagatggattgcacgcaggttc |
| | tccggccgcttgggtggagaggctattcggctatgactg |
| | ggcacaacagacaatcggctgctctgatgccgccgtgtt |
| | ccggctgtcagcgcaggggcgcccggttctttttgtcaa |
| | gaccgacctgtccggtgccctgaatgaactgcaggacga |
| | ggcagcgcggctatcgtggctggccacgacgggcgttcc |
| | ttgcgcagctgtgctcgacgttgtcactgaagcgggaag |
| | ggactggctgctattgggcgaagtgccggggcaggatct |
| | cctgtcatctcaccttgctcctgccgagaaagtatccat |
| | catggctgatgcaatgcggcggctgcatacgcttgatcc |
| | ggctacctgcccattcgaccaccaagcgaaacatcgcat |
| | cgagcgagcacgtactcggatggaagccggtcttgtcaa |
| | tcaggatgatctggacgaagagcatcaggggctcgcgcc |
| | agccgaactgttcgccaggctcaaggcgcgcatgcccga |
| | cggcgaggatctcgtcgtgacccatggcgatgcctgctt |
| | gccgaatatcatggtggaaaatggccgcttttctggatt |
| | catcgactgtggccggctgggtgtggcggatcgctatca |
| | ggacatagcgttggctacccgtgatattgctgaagagct |
| | tggcggcgaatgggctgaccgcttcctcgtgctttacgg |
| | tatcgccgctcccgattcgcagcgcatcgccttctatcg |
| | ccttcttgacgagttcttctgaggggatcaattcTCTAG |
| | ATGCATGCTCGAGCGGCCGCCAGTGTGATGGATATCTGC |
| | AGAATTCGCCCTtCCAGGCGTTGAAGTCGTCGTGTCCTC |
| | AGGTAAGAACGGCCCTCCAGGGCCTTTAATTTCTGCTCT |
| | CGTCTGTGGGCTTTTCTGACTCTGATCCTCGGGAGGCGT |
| | CTGTGCCCCCCCCGGGGATGAGGCCGGCTTGCCAGGAGG |
| | GGTCAGGGACCAGGAGCCTGTGGGAAGTTCTGACGGGGG |
| | CTGCAGGCGGGAAGGGCCCCACCGGGGGGCGAGCCCCAG |
| | GCCGCTGGGCGGCAGGAGACCCGTGAGAGTGCGCCTTGA |
| | GGAGGGTGTCTGCGGAACCACGAACGCCCGCCGGGAAGG |
| | GCTTGCTGCAATGCGGTCTTCAGACGGGAGGCGTCTTCT |
| | GCCCTCACCGTCTTTCAAGCCCTTGTGGGTCTGAAAGAG |
| | CCATGTCGGAGAGAGAAGGGACAGGCCTGTGGCGAGCTG |
| | GCCGAGAGCGGGCAGCCCCGGGGGAGAGCGGGGCGATCG |
| | GCGTGGGCTCTGTGAGGCCAGGTCCAAGGGAGGACGTGT |
| | GGTCCTCGTGACAGGTGCACTTGCGAAACCTTAGAAGAC |
| | GGGGTATGTTGGAAGCGGCTCCTGATGTTTAAGAAAAGG |
| | GAGACTGTAAAGTGAGCAGAGTCCTCAAGTGTGTTAAGG |
| | TTTTAAAGGTCAAAGTGTTTTAAAGCTTTGTGAGTGCAG |
| | TTAGCAAGCGTGCGGGGAGTGAATGGGGTGCCAGGGTGG |
| | CCGAGAGGCAGTACGAGGGCCGTGCCGTCCTGTAATTCA |
| | GGGCTTAGTTTTGCAGAATAAAGTCGGCCTGTTTTCTAA |
| | AAGCATTGGTGGTGCTGAGGTGGTGGAGGAGGCCGCGGG |
| | CAGCCCTGGCCACCTGCAGCAGGTGGCAGGAAGCAGGTC |
| | GGCCAAGAGGCTATTTTAGGAAGCCAGAAAACACGGTCG |
| | ATGAATTTATAGCTTCTGGTTTCCAGGAGGTGGTTGGGC |
| | ATGGCTTTGCGCAGCGCCACAGAACCGAAAGTGCCCACT |
| | GAGAAAAAACAACTCCTGCTTAATTTGCATTTTTCTAAA |
| | AGAAGAAACAGAGGCTGACGGAAACTGGAAAGTTCCTGT |
| | TTTAACTACTCGAATTGAGTTTTCGGTCTTAGCTTATCA |
| | ACTGCTCACTTAGATTCATTTTCAAAGTAAACGTTTAAG |
| | AGCCGAGGCATTCCTATCCTCTTCTAAGGCGTTATTCCT |
| | GGAGGCTCATTCACCGCCAGCACCTCCGCTGCCTGCAGG |
| | CATTGCTGTCAGGGTCACCGTGACGGCGCGCACGATTTT |
| | CAGTTGGCCCGCTTCCCCTCGTGATTAGGACAGACGCGG |
| | GCACTCTGGCCCAGCCGTCTTGGCTCAGTATCTGCAGGC |
| | GTCCGTCTCGGGACGGAGCTCAGGGGAAGAGCGTGACTC |
| | CAGTTGAACGTGATAGTCGGTGCGTTGAGAGGAGACCCA |
| | GTCGGGTGTCGAGTCAGAAGGGGCCCGGGGCCCGAGGCC |
| | CTGGGCAGGACGGGCCGTGCCCTGGATCACGGGCCCAGC |
| | GTCCTAGAGGCAGGACTCTGGTGGAGAGTGTGAGGGTGC |
| | CTGGGGCCCCTCCGGAGCTGGGGCCGTGCGGTGCAGGTT |
| | GGGCTCTCGGCGCGGTGTTGGCTGTTTCTGCGGGATTTG |
| | GAGGAATTCTTCCAGTGATGGGAGTCGCCAGTGACCGGG |
| | CACCAGGGTGGTAAGAGGGAGGCCGCCGTCGTGGCCAGA |
| | GCAGGTGGGAGGGTTCGGTAAAAGGCTCGCCCGTTTCCT |
| | TTAATGAGGACTTTTCCTGGAGGGCATTTAGTCTAGTCG |
| | GGACCGTTTTCGACTCGGGAAGAGGGATGCGGAGGAGGG |
| | CATGTGCCCAGGAGCCGAAGGCGCCGCGGGGAGAAGCCC |
| | AGGGCTCTCCTGTCCCCACAGAGGCGACGCCACTGCCGC |
| | AGACAGACAGGGCCTTTCCCTCTGATGACGGCAAAGGCG |
| | CCTCGGCTCTTGCGGGGTGCTGGGGGGGAGTCGCCCCGA |
| | AGCCGCTCACCCAGAGGCCTGAGGGGTGAGACTGACCGA |
| | TGCCTCTTGGCCGGGCCTGGGGCCGGACCGAGGGGGACT |
| | CCGTGGAGGCAGGGCGATGGTGGCTGCGGGAGGGAACCG |
| | ACCCTGGGCCGAGCCCGGCTTGGCGATTCCCGGGCGAGG |
| | GCCCTCAGGCGAGGCGAGTGGGTCCGGCGGAACCACCCT |
| | TTCTGGCCAGCGCCACAGGGCTCTCGGGACTGTCCGGGG |
| | CGACGCTGGGCTGCCCGTGGCAGGCCTGGGCTGACCTGG |
| | ACTTCACCAGACAGAACAGGGCTTTCAGGGCTGAGCTGA |
| | GCCAGGTTTAGCGAGGCCAAGTGGGGCTGAACCAGGCTC |
| | AACTGGCCTGAGCTGGGTTGAGCTGGGCTGACCTGGGCT |
| | GAGCTGAGCTGGGGTGGGCTGGGCTGGGCTGGGCTGGGG |
| | TGGGCTGGACTGGCTGAGCTGAGCTGGGTTGAGCTGAGC |
| | TGAGCTGGCCTGGGTTGAGCTGGGCTGGGTTGAGCTGAG |
| | CTGGGTTGAGCTGGGTTGAGCTGGGTTGATCTGAGCTGA |
| | GGTGGGCTGAGCTGAGCTAGGCTGGGGTGAGGTGGGGTG |
| | ATCTGAGCTGAGCTGGGCTGAGCTGAGCTAGGCTGGGGT |
| | GAGCTGGGCTGAGCTGGTTTGAGTTGGGTTGAGCTGAGC |
| | TGAGCTGGGCTGTGCTGGCTGAGCTAGGCTGAGCTAGGC |
| | TAGGTTGAGCTGGGCTGGGCTGAGCTGAGCTAGGCTGGG |
| | CTGATTTGGGCTGAGCTGAGCTGAGCTAGGCTGCGTTGA |
| | GCTGGCTGGGCTGGATTGAGCTGGCTGAGCTGGCTGAGC |
| | TGGGCTGAGCTGGCCTGGGTTGAGCTGAGCTGGACTGGT |
| | TTGAGCTGGGTCGATCTGGGTTGAGCTGTCCTGGGTTGA |
| | GCTGGGCTGGGTTGAGCTGAGCTGGGTTGAGCTGGGCTC |
| | AGCAGAGCTGGGTTGGGCTGAGCTGGGTTGAGCTGAGCT |
| | GGGCTGAGCTGGCCTGGGTTGAGCTGGGCTGAGCTGAGC |
| | TGGGCTGAGCTGGCCTGTGTTGAGCTGGGCTGGGTTGAG |
| | CTGGGCTGAGCTGGATTGAGCTGGGTTGAGCTGAGCTGG |
| | GCTGGGCTGTGCTGACTGAGCTGGGGTGAGCTAGGCTGG |
| | GGTGAGCTGGGCTGAGCTGATCCGAGCTAGGCTGGGCTG |
| | GTTTGGGCTGAGCTGAGCTGAGCTAGGCTGGATTGATCT |
| | GGCTGAGCTGGGTTGAGCTGAGCTGGGCTGAGCTGGTCT |
| | GAGCTGGCCTGGGTCGAGCTGAGCTGGACTGGTTTGAGC |
| | TGGGTCGATCTGGGCTGAGCTGGCCTGGGTTGAGCTGGG |
| | CTGGGTTGAGCTGAGCTGGGTTGAGCTGGGGTGAGCTGA |
| | GGGCTGGGGTGAGGTGGGGTGAACTAGCCTAGCTAGGTT |
| | GGGCTGAGCTGGGCTGGTTTGGGCTGAGCTGAGCTGAGG |
| | TAGGGTGCATTGAGCAGGCTGAGCTGGGCTGAGCAGGCG |
| | TGGGGTGAGCTGGGCTAGGTGGAGCTGAGCTGGGTCGAG |
| | CTGAGTTGGGCTGAGCTGGCCTGGGTTGAGGTAGGCTGA |
| | GCTGAGCTGAGCTAGGCTGGGTTGAGCTGGCTGGGCTGG |
| | TTTGCGCTGGGTCAAGCTGGGCCGAGCTGGCCTGGGTTG |
| | AGCTGGGCTCGGTTGAGCTGGGCTGAGCTGAGCCGACCT |
| | AGGCTGGGATGAGCTGGGCTGATTTGGGCTGAGCTGAGC |
| | TGAGCTAGGCTGCATTGAGCAGGCTGAGGTGGGGCTGGA |
| | GCCTGGCGTGGGGTGAGCTGGGCTGAGCTGCGCTGAGCT |
| | AGGCTGGGTTGAGCTGGCTGGGCTGGTTTGCGCTGGGTC |
| | AAGCTGGGCCGAGCTGGCCTGGGATGAGCTGGGCCGGTT |
| | TGGGCTGAGCTGAGCTGAGGTAGGCTGCATTGAGCAGGG |
| | TGAGCTGGGGTGAGCTGGGCTGGGGTGAGCTGGGCTGAG |
| | CTAAGCTGAGCTGGGCTGGTTTGGGGTGAGGTGGCTGAG |
| | CTGGGTCCTGGTGAGCTGGGCTGAGGTGACCAGGGGTGA |
| | GGTGGGCTGAGTTAGGCTGGGCTCAGCTAGGCTGGGTTG |
| | ATCTGGCAGGGCTGGTTTGCGCTGGGTCAAGCTCCCGGG |
| | AGATGGCCTGGGATGAGCTGGGCTGGTTTGGGCTGAGCT |
| | GAGCTGAGCTGAGCTAGGCTGCATTGAGCAGGCTGAGCT |
| | GGGCTGAGCTGGCCTGGGGTGAGGTGGGCTGGGTGGAGC |
| | TGAGCTGGGCTGAACTGGGGTAAGCTGGCTGAGCTGGAT |
| | CGAGCTGAGCTGGGGTGAGGTGGCCTGGGGTTAGCTGGG |
| | CTGAGCTGAGCTGAGCTAGGGTGGGTTGAGCTGGCTGGG |
| | CTGGTTTGCGGTGGGTCAAGGTGGGCGGAGGTGGCCTGG |
| | GTTGAGCTGGGCTGGGCTGAGCTGAGCTAGGCTGGGTTG |
| | AGCTGGGCTGGGCTGAGCTGAGCTAGGCTGCATTGAGCT |
| | GGCTGGGATGGATTGAGCTGGCTGAGCTGGCTGAGCTGG |
| | CTGAGCTGGGCTGAGCTGGCCTGGGTTGAGCTGGGCTGG |
| | GTTGAGCTGAGCTGGGCTGAGCTGGGCTCAGCAGAGCTG |
| | GGTTGAGCTGAGCTGGGTTGAGGTGGGGTGAGCTGGGGT |
| | GAGCAGAGCTGGGTTGAGCTGAGCTGGGTTGAGCTGGGC |
| | TCGAGCAGAGCTGGGTTGAGCTGAGCTGGGTTGAGCTGG |
| | GCTCAGCAGAGCTGGGTTGAGCTGAGCTGGGTTGAGCTG |
| | GGCTGAGCTAGCTGGGCTCAGCTAGGGTGGGTTGAGCTG |
| | AGCTGGGCTGAAGTGGGCTGAGCTGGGCTGAAGTGGGGT |
| | GAGCTGGGCTGAGCTGGGGTGAGCAGAGCTGGGCTGAGC |
| | AGAGGTGGGTTGGTCTGAGCTGGGTTGAGCTGGGGTGAG |
| | CTGGGCTGAGCAGAGTTGGGTTGAGCTGAGCTGGGTTCA |
| | GCTGGGCTGAGCTAGGCTGGGTTGAGCTGGGTTGAGTTG |
| | GGCTGAGCTGGGCTGGGTTGAGCGGAGCTGGGCTGAACT |
| | GGGCTGAGCTGGGCTGAGCGGAACTGGGTTGATGTGAAT |
| | TGAGCTGGGCTGAGCCGGGCTGAGCCGGGCTGAGCTGGG |
| | CTAGGTTGAGCTTGGGTGAGCTTGCCTCAGCTGGTCTGA |
| | GCTAGGTTGGGTGGAGCTAGGCTGGATTGAGGTGGGCTG |
| | AGCTGAGCTGATCTGGCCTCAGCTGGGCTGAGGTAGGCT |
| | GAACTGGGGTGTGCTGGGCTGAGGTGAGCTGAGCCAGTT |
| | TGAGCTGGGTTGAGCTGGGCTGAGCTGGGCTGTGTTGAT |
| | CTTTCCTGAACTGGGCTGAGCTGGGCTGAGCTGGCCTAG |
| | CTGGATTGAACGGGGGTAAGCTGGGCCAGGCTGGACTGG |
| | GCTGAGGTGAGCTAGGCTGAGCTGAGTTGAATTGGGTTA |
| | GCTGGGCTGAGATGGGCTGGAGCTGGGCTGAGCTGGGTT |
| | GAGCCAGGTCGGACTGGGTTACCCTGGGCCACACTGGGG |
| | TGAGCTGGGCGGAGCTCGATTAACCTGGTCAGGCTGAGT |
| | CGGGTCCAGCAGACATGCGCTGGCCAGGCTGGCTTGACC |
| | TGGACACGTTCGATGAGCTGCCTTGGGATGGTTCACCTC |
| | AGCTGAGCCAGGTGGCTCCAGCTGGGCTGAGCTGGTGAC |
| | CCTGGGTGACCTCGGTGACCAGGTTGTCCTGAGTCCGGG |
| | CCAAGCCGAGGCTGCATCAGAGTCGCCAGACCCAAGGCC |
| | TGGGGCCCGGGTGGCAAGCCAGGGGCGGTGAAGGCTGGG |
| | GTGGCAGGACTGTCCCGGAAGGAGGTGCACGTGGAGCCG |
| | CCCGGACCCCGACCGGCAGGACCTGGAAAGACGGGTCTC |
| | ACTCCCCTTTCTCTTCTGTCCCCTCTCGGGTGCTCAGAG |
| | AGCCAGTCTGCCCCGAATCTGTACCCCCTCGTCTCCTGC |
| | GTCAGCCCCCCGTCCGATGAGAGCCTGGTGGCCCTGGGC |
| | TGCCTGGCCCGGGACTTCCTGCCCAGCTCCGTCACCTTC |
| | TCCTGGAA |

Porcine Kappa Chain Targeting

In particular embodiments of the present invention, targeting vectors are provided to target the porcine kappa chain locus. In one particular embodiment, the targeting vector can contain 5′ and 3′ recombination arms that contain homologous sequence to the 3′ and 5′ flanking sequence of the constant region of the porcine immunoglobulin kappa chain locus. Since the present invention discovered that there is only one constant region of the porcine immunoglobulin kappa light chain locus, this will prevent the expression of a functional porcine kappa light chain immunoglobulin. In a specific embodiment, the targeting vector can contain a 5′ recombination arm that contains sequence homologous to genomic sequence 5′ of the constant region, optionally including the joining region, and a 3′ recombination arm that contains sequence homologous to genomic sequence 3′ of the constant region, optionally including at least part of the enhancer region (a “Kappa constant targeting construct”), see for example, FIG. 2. Further, this kappa constant targeting construct can also contain a selectable marker gene that is located between the 5′ and 3′ recombination arms, see for example, Seq ID No 20 and FIG. 2. In other embodiments, the targeting vector can contain a 5′ recombination arm that contains sequence homologous to genomic sequence 5′ of the joining region, and a 3′ recombination arm that contains sequence homologous to genomic sequence 3′ of the joining region of the porcine kappa light chain locus. In other embodiments, the 5′ arm of the targeting vector can include Seq ID No 12 and/or Seq ID No 25 or any contiguous sequence or fragment thereof. In another embodiment, the 3′ arm of the targeting vector can include Seq ID No 15, 16 and/or 19 or any contiguous sequence or fragment thereof.

In further embodiments, the targeting vector can include, but is not limited to any of the following sequences: the coding region of kappa light chain is represented, for example by residues 1-549 of Seq ID No 30 and 10026-10549 of Seq ID No 30, whereas the intronic sequence is represented, for example, by residues 550-10025 of Seq ID No 30, the Joining region of kappa light chain is represented, for example, by residues 5822-7207 of Seq ID No 30 (for example, J1:5822-5859 of Seq ID No 30, J2:6180-6218 of Seq ID No 30, J3:6486-6523 of Seq ID No 30, J4:6826-6863 of Seq ID No 30, J5:7170-7207 of Seq ID No 30), the Constant Region is represented by the following residues: 10026-10549 of Seq ID No 30 (C exon) and 10026-10354 of Seq ID No 30 (C coding), 10524-10529 of Seq ID No 30 (Poly(A) signal) and 11160-11264 of Seq ID No 30 (SINE element) or any fragment or combination thereof. Still further, any contiguous sequence at least about 17, 20, 30, 40, 50, 100, 150, 200. or 300 nucleotides of Seq ID No 30 or fragment and/or combination thereof can be used as targeting sequence for the heavy chain targeting vector. It is understood that in general when designing a targeting construct one targeting arm will be 5′ of the other targeting arm.

| | |
|---|---|
| Seq ID No.20 | ctcaaacgtaagtggctttttccgactgattctttgc |
| | tgtttctaattgttggttggctttttgtccatttttc |
| | agtgttttcatcgaattagttgtcagggaccaaacaa |
| | attgccttcccagattaggtaccagggaggggacatt |
| | gctgcatgggagaccagagggtggctaatttttaacg |
| | tttccaagccaaaataactggggaagggggcttgctg |
| | tcctgtgagggtaggtttttatagaagtggaagttaa |
| | ggggaaatcgctatggttcacttttggctcggggacc |
| | aaagtggagcccaaaattgagtacattttccatcaat |
| | tatttgtgagatttttgtcctgttgtgtcatttgtgc |
| | aagtttttgacattttggttgaatgagccattcccag |
| | ggacccaaaaggatgagaccgaaaagtagaaaagagc |
| | caacttttaagctgagcagacagaccgaattgttgag |
| | tttgtgaggagagtagggtttgtagggagaaagggga |
| | acagatcgctggctttttctctgaattagcctttctc |
| | atgggactggcttcagagggggtttttgatgagggaa |
| | gtgttctagagccttaactgtgggttgtgttcggtag |
| | cgggaccaagctggaaatcaaacgtaagtgcactttt |
| | ctactcctttttctttcttatacgggtgtgaaattgg |
| | ggacttttcatgtttggagtatgagttgaggtcagtt |
| | ctgaagagagtgggactcatccaaaaatctgaggagt |
| | aagggtcagaacagagttgtctcatggaagaacaaag |
| | acctagttagttgatgaggcagctaaatgagtcagtt |
| | gacttgggatccaaatggccagacttcgtctgtaacc |
| | aacaatctaatgagatgtagcagcaaaaagagatttc |
| | cattgaggggaaagtaaaattgttaatattgtggatc |
| | acctttggtgaagggacatccgtggagattgaacgta |
| | agtattttttctctactaccttctgaaatttgtctaa |
| | atgccagtgttgacttttagaggcttaagtgtcagtt |
| | ttgtgaaaaatgggtaaacaagagcatttcatattta |
| | ttatcagtttcaaaagttaaactcagctccaaaaatg |
| | aatttgtagacaaaaagattaatttaagccaaattga |
| | atgattcaaaggaaaaaaaaattagtgtagatgaaaa |
| | aggaattcttacagctccaaagagcaaaagcgaatta |
| | attttctttgaactttgccaaatcttgtaaatgattt |
| | ttgttctttacaatttaaaaaggttagagaaatgtat |
| | ttcttagtctgttttctctcttctgtctgataaatta |
| | ttatatgagataaaaatgaaaattaataggatgtgct |
| | aaaaaatcagtaagaagttagaaaaatatatgtttat |
| | gttaaagttgccacttaattgagaatcagaagcaatg |
| | ttatttttaaagtctaaaatgagagataaactgtcaa |
| | tacttaaattctgcagagattctatatcttgacagat |
| | atctcctttttcaaaaatccaatttctatggtagact |
| | aaatttgaaatgatcttcctcataatggagggaaaag |
| | atggactgaccccaaaagctcagattt*aagaaaacc |
| | tgtttaag*gaaagaaaataaaagaactgcatttttt |
| | aaaggcccatgaatttgtagaaaaataggaaatattt |
| | taataagtgtattcttttattttcctgttattacttg |
| | atggtgtttttataccgccaaggaggccgtggcaccg |
| | tcagtgtgatctgtagaccccatggcggccttttttc |
| | gcgattgaatgaccttggcggtgggtccccagggctc |
| | tggtggcagcgcaccagccgctaaaagccgctaaaaa |
| | ctgccgctaaaggccacagcaaccccgcgaccgcccg |
| | ttcaactgtgctgacacagtgatacagataatgtcgc |
| | taacagaggagaatagaaatatgacgggcacacgcta |
| | atgtggggaaaagagggagaagcctgatttttatttt |
| | ttagagattctagagataaaattcccagtattatatc |
| | cttttaataaaaaatttctattaggagattataaaga |
| | atttaaagctatttttttaagtggggtgtaattcttt |
| | cagtagtctcttgtcaaatggatttaagtaatagagg |
| | cttaatccaaatgagagaaatagacgcataacccttt |
| | caaggcaaaagctacaagagcaaaaattgaacacagc |
| | agccagccatctagccactcagattttgatcagtttt |
| | actgagtttgaagtaaatatcatgaaggtataattgc |
| | tgataaaaaaataagatacaggtgtgacacatcttta |
| | agtttcagaaatttaatggcttcagtaggattatatt |
| | tcacgtatacaaagtatctaagcagataaaaatgcca |
| | ttaatggaaacttaatagaaatatatttttaaattcc |
| | ttcattctgtgacagaaattttctaatctgggtcttt |
| | taatcacctaccctttgaaagagtttagtaatttgct |
| | atttgccatcgctgtttactccagctaatttcaaaag |
| | tgatacttgagaaagattatttttggtttgcaaccac |
| | ctggcaggactattttagggccattttaaaactcttt |
| | tcaaactaagtattttaaactgttctaaaccatttag |
| | ggccttttaaaaatcttttcatgaatttcaaacttcg |
| | ttaaaagttattaaggtgtctggcaagaacttcctta |
| | tcaaatatgctaatagtttaatctgttaatgcaggat |
| | ataaaattaaagtgatcaaggcttgacccaaacagga |
| | gtatcttcatagcatatttcccctcctttttttctag |
| | aattcatatgattttgctgccaaggctattttatata |
| | atctctggaaaaaaaatagtaatgaaggttaaaagag |
| | aagaaaatatcagaacattaagaattcggtattttac |
| | taactgcttggttaacatgaaggtttttattttatta |
| | aggtttctatctttataaaaatctgttcccttttctg |
| | ctgatttctccaagcaaaagattcttgatttgttttt |
| | taactcttactctcccacccaagggcctgaatgccca |
| | caaaggggacttccaggaggccatctggcagctgctc |
| | accgtcagaagtgaagccagccagttcctcctgggca |
| | ggtggccaaaattacagttgacccctcctggtctggc |
| | tgaaccttgccccatatggtgacagccatctggccag |
| | ggcccaggtctccctctgaagcctttgggaggagagg |
| | gagagtggctggcccgatcacagatgcggaaggggct |
| | gactcctcaaccggggtgcagactctgcagggtgggt |
| | ctgggcccaacacacccaaagcacgcccaggaaggaa |
| | aggcagcttggtatcactgcccagagctaggagaggc |
| | accgggaaaatgatctgtccaagacccgttcttgctt |
| | ctaaactccgagggggtcagatgaagtggttttgttt |
| | cttggcctgaagcatcgtgttccctgcaagaagcggg |
| | gaacacagaggaaggagagaaaagatgaactgaacaa |
| | agcatgcaaggcaaaaaaggGGGTCTAGCCGCGGTCT |
| | AGGAAGCTTTCTAGGGTACCTCTAGGGATCCCGGCGC |
| | GCCCTACCGGGTAGGGGAGGCGCTTTTCCCAAGGCAG |
| | TCTGGAGCATGCGCTTTAGCAGCCCCGCTGGGCACTT |
| | GGCGCTACACAAGTGGCCTCTGGCCTCGCACACATTG |
| | CACATCCACCGGTAGGGGCCAACCGGCTGCGTTCTTT |
| | GGTGGCCGCTTCGCGCCACCTTGTACTGCTCCCCTAG |
| | TCAGGAAGTTCGCCGCGGCCCCGCAGCTCGCGTCGTG |
| | GAGGACGTGACAAATGGAAGTAGCACGTCTGACTAGT |
| | CTCGTGCAGATGGACAGCACCGCTGAGCAATGGAAGC |
| | GGGTAGGCCTTTGGGGCAGCGGCCAATAGCAGCTTTG |
| | GCTCCTTCGCTTTCTGGGCTCAGAGGCTGGGAAGGGG |
| | TGGGTCCGGGGGCGGGCTCAGGGGCGGGGTCAGGGGC |
| | GGGGCGGGCGCCCGAAGGTCCTCCGGAAGCCCGGCAT |
| | TCTGCACGCTTCAAAAGCGCACGTCTGCCGCGCTGTT |
| | CTCCTCTTCCTCATCTCCGGGCCTTTCGACCTGCAGC |
| | CAATATGGGATCGGCCATTGAACAAGATGGATTGCAC |
| | GCAGGTTCTCCGGCCGCTTGGGTGGAGAGGCTATTCG |
| | GCTATGACTGGGCACAACAGACAATCGGCTGCTCTGA |
| | TGCCGCCGTGTTCCGGCTGTCAGCGCAGGGGCGCCCG |
| | GTTCTTTTTGTCAAGACCGACCTGTCCGGTGCCCTGA |
| | ATGAACTGCAGGACGAGGCAGCGCGGCTATCGTGGCT |
| | GGCCACGACGGGCGTTCCTTGCGCAGCTGTGCTCGAC |
| | GTTGTCACTGAAGCGGGAAGGGACTGGCTGCTATTGG |
| | GCGAAGTGCCGGGGCAGGATCTCCTGTCATCTCACCT |
| | TGCTCCTGCCGAGAAAGTATCCATCATGGCTGATGCA |
| | ATGCGGCGGGTGCATACGCTTGATCCGGGTAGGTGCC |
| | GATTCGACCACCAAGCGAAACATCGCATCGAGCGAGC |
| | ACGTACTCGGATGGAAGGCGGTCTTGTCAATCAGGAT |
| | GATCTGGACGAAGAGCATGAGGGGCTCGCGGCAGCCG |
| | AACTGTTCGCCAGGCTCAAGGCGCGCATGCCCGACGG |
| | CGAGGATCTCGTCGTGACCCATGGCGATGCCTGCTTG |
| | CCGAATATCATGGTGGAAAATGGCCGCTTTTCTGGAT |
| | TCATCGACTGTGGCCGGCTGGGTGTGGCGGATCGCTA |
| | TCAGGACATAGCGTTGGCTACCCGTGATATTGCTGAA |
| | GAGCTTGGCGGCGAATGGGCTGACCGCTTCCTCGTGC |
| | TTTACGGTATCGCCGCTCCCGATTCGCAGCGCATCGC |
| | CTTCTATCGCCTTCTTGACGAGTTCTTCTGAGGGGAT |
| | CAATTCTCTAGAGCTCGCTGATCAGCCTCGACTGTGC |
| | CTTCTAGTTGCCAGCCATCTGTTGTTTGCCCCTCCCC |
| | CGTGCCTTCCTTGACCCTGGAAGGTGCCACTCCCACT |
| | GTCCTTTCCTAATAAAATGAGGAAATTGCATCGCATT |
| | GTCTGAGTAGGTGTCATTCTATTCTGGGGGGTGGGGT |
| | GGGGCAGGACAGCAAGGGGGAGGATTGGGAAGACAAT |
| | AGCAGGCATGCTGGGGATGGGGTGGGCTCTATGGCTT |
| | CTGAGGGGGAAAGAACCAGCTGGGGGCGCGCCCctcg |
| | agcggccgccagtgtgatggatatctgcagaattcgc |
| | ccttggatcaaacacgcatcctcatggacaatatgtt |
| | gggttcttagcctgctgagacacaacaggaactcccc |
| | tggcaccactttagaggccagagaaacagcacagata |
| | aaattccctgccctcatgaagcttatagtctagctgg |
| | ggagatatcataggcaagataaacacatacaaataca |
| | tcatcttaggtaataatatatactaaggagaaaatta |
| | caggggagaaagaggacaggaattgctagggtaggat |
| | tataagttcagatagttcatcaggaacactgttgctg |
| | agaagataacatttaggtaaagaccgaagtagtaagg |
| | aaatggaccgtgtgcctaagtgggtaagaccattcta |
| | ggcagcaggaacagcgatgaaagcactgaggtgggtg |
| | ttcactgcacagagttgttcactgcacagagttgtgt |
| | ggggaggggtaggtcttgcaggctcttatggtcacag |
| | gaagaattgttttactcccaccgagatgaaggttggt |
| | ggattttgagcagaagaataattctgcctggtttata |
| | tataacaggatttccctgggtgctctgatgagaataa |
| | tctgtcaggggtgggatagggagagatatggcaatag |
| | gagccttggctaggagcccacgacaataattccaagt |
| | gagaggtggtgctgcattgaaagcaggactaacaaga |
| | cctgctgacagtgtggatgtagaaaaagatagaggag |
| | acgaaggtgcatctagggttttctgcctgaggaatta |
| | gaaagataaagctaaagcttatagaagatgcagcgct |
| | ctggggagaaagaccagcagctcagttttgatccatc |
| | tggaattaattttggcataaagtatgaggtatgtggg |
| | ttaacattatttgttttttttttttccatgtagctat |
| | ccaactgtcccagcatcatttattttaaaagactttc |
| | ctttcccctattggattgttttggcaccttcactgaa |
| | gatcaactgagcataaaattgggtctatttctaagct |
| | cttgattccattccatgacctatttgttcatctttac |
| | cccagtagacactgccttgatgattaaagcccctgtt |
| | accatgtctgttttggacatggtaaatctgagatgcc |
| | tattagccaaccaagcaagcacggcccttagagagct |
| | agatatgagagcctggaattcagacgagaaaggtcag |
| | tcctagagacatacatgtagtgccatcaccatgcgga |
| | tggtgttaaaagccatcagactgcaacagactgtgag |
| | agggtaccaagctagagagcatggatagagaaaccca |
| | agcactgagctgggaggtgctcctacattaagagatt |
| | agtgagatgaaggactgagaagattgatcagagaaga |
| | aggaaaatcaggaaaatggtgctgtcctgaaaatcca |
| | agggaagagatgttccaaagaggagaaaactgatcag |
| | ttgtcagctagcgtcaattgggatgaaaatggaccat |
| | tggacagagggatgtagtgggtcatgggtgaatagat |
| | aagagcagcttctatagaatggcaggggcaaaattct |
| | catctgatcggcatgggttctaaagaaaacgggaaga |
| | aaaaattgagtgcatgaccagtcccttcaagtagaga |
| | ggtggaaaagggaaggaggaaaatgaggccacgacaa |
| | catgagagaaatgacagcatttttaaaaattttttat |
| | tttattttatttatttatttttgctttttagggctgc |
| | ccctgcaacatatggaggttcccaggttaggggtcta |
| | atcagagctatagctgccagcctacaccacagccata |
| | gcaatgccagatctacatgacctacaccacagctcac |
| | agcaacgccggatccttaacccactgagtgaggccag |
| | agatcaaacccatatccttatggatactagtcaggtt |
| | cattaccactgagccaaaatgggaaatcctgagtaat |
| | gacagcattttttaatgtgccaggaagcaaaacttgc |
| | caccccgaaatgtctctcaggcatgtggattattttg |
| | agctgaaaacgattaaggcccaaaaaacacaagaaga |
| | aatgtggaccttcccccaacagcctaaaaaatttaga |
| | ttgagggcctgttcccagaatagagctattgccagac |
| | ttgtctacagaggctaagggctaggtgtggtggggaa |
| | accctcagagatcagagggacgtttatgtaccaagca |
| | ttgacatttccatctccatgcgaatggccttcttccc |
| | ctctgtagccccaaaccaccacccccaaaatcttctt |
| | ctgtctttagctgaagatggtgttgaaggtgatagtt |
| | tcagccactttggcgagttcctcagttgttctgggtc |
| | tttcctccTgatccacattattcgactgtgtttgatt |
| | ttctcctgtttatctgtctcattggcacccatttcat |
| | tcttagaccagcccaaagaacctagaagagtgaagga |
| | aaatttcttccaccctgacaaatgctaaatgagaatc |
| | accgcagtagaggaaaatgatctggtgctgcgggaga |
| | tagaagagaaaatcgctggagagatgtcactgagtag |
| | gtgagatgggaaaggggtgacacaggtggaggtgttg |
| | ccctcagctaggaagacagacagttcacagaagagaa |
| | gcgggtgtccgtggacatcttgcctcatggatgagga |
| | aaccgaggctaagaaagactgcaaaagaaaggtaagg |
| | attgcagagaggtcgatccatgactaaaatcacagta |
| | accaaccccaaaccaccatgttttctcctagtctggc |
| | acgtggcaggtactgtgtaggttttcaatattattgg |
| | tttgtaacagtacctattaggcctccatcccctcctc |
| | taatactaacaaaagtgtgagactggtcagtgaaaaa |
| | tggtcttctttctctatgaatctttctcaagaagata |
| | cataactttttattttatcataggcttgaagagcaaa |
| | tgagaaacagcctccaacctatgacaccgtaacaaaa |
| | tgtttatgatcagtgaagggcaagaaacaaaacatac |
| | acagtaaagaccctccataatattgtgggtggcccaa |
| | cacaggccaggttgtaaaagctttttattctttgata |
| | gaggaatggatagtaatgtttcaacctggacagagat |
| | catgttcactgaatccttccaaaaattcatgggtagt |
| | ttgaattataaggaaaataagacttaggataaatact |
| | ttgtccaagatcccagagttaatgccaaaatcagttt |
| | tcagactccaggcagcctgatcaagagcctaaacttt |
| | aaagacacagtcccttaataactactattcacagttg |
| | cactttcagggcgcaaagactcattgaatcctacaat |
| | agaatgagtttagatatcaaatctctcagtaatagat |
| | gaggagactaaatagcgggcatgacctggtcacttaa |
| | agacagaattgagattcaaggctagtgttctttctac |
| | ctgttttgtttctacaagatgtagcaatgcgctaatt |
| | acagacctctcagggaaggaa |

Porcine Lambda Chain Targeting

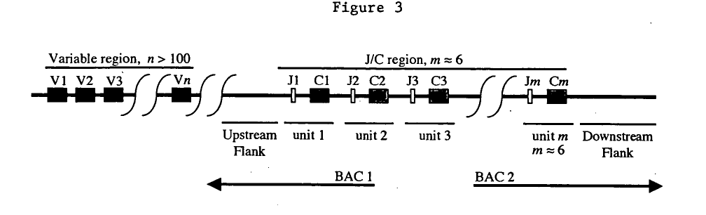

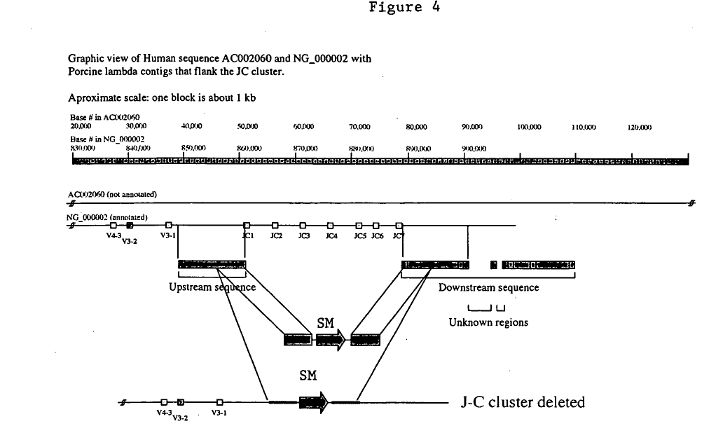

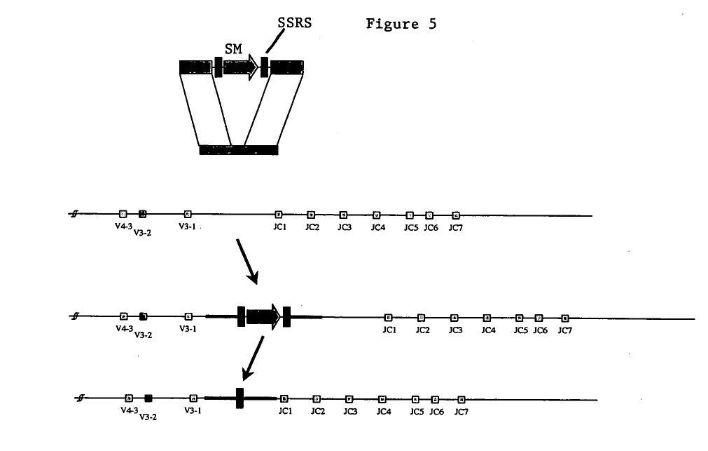

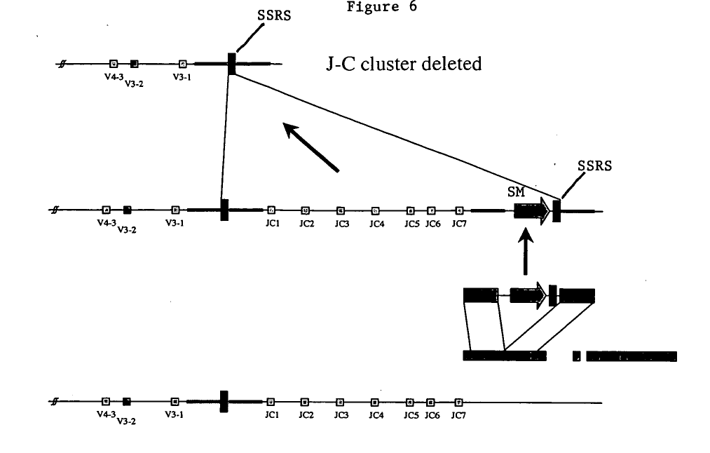

In particular embodiments of the present invention, targeting vectors are provided to target the porcine heavy chain locus. In one embodiment, lambda can be targeted by designing a targeting construct that contains a 5′ arm containing sequence located 5′ to the first JC cluster and a 3′ arm containing sequence 3′ to the last JC cluster, thus preventing functional expression of the lambda locus (see, FIGS. 3-4). In one embodiment, the targeting vector can contain any contiguous sequence (such as about 17, 20, 30, 40, 50, 75, 100, 200, 300 or.5000 nucleotides of contiguous sequence) or fragment thereof Seq ID No 28. In one embodiment, the 5′ targeting arm can contain Seq ID No. 32, which includes 5′ flanking sequence to the first lambda J/C region of the porcine lambda light chain genomic sequence or any contiguous sequence (such as about 17, 20, 30, 40, 50, 75, 100, 200, 300 or 5000 nucleotides of contiguous sequence) or fragment thereof (see also, for example FIG. 5). In another embodiment, the 3′ targeting arm can contain, but is not limited to one or more of the following: Seq ID No. 33, which includes 3′ flanking sequence to the J/C cluster region of the porcine lambda light chain genomic sequence, from approximately 200 base pairs downstream of lambda J/C; Seq ID No.34, which includes 3′ flanking sequence to the J/C cluster region of the porcine lambda light chain genomic sequence, approximately 11.8 Kb downstream of the J/C cluster, near the enhancer; Seq ID No. 35, which includes approximately 12 Kb downstream of lambda, including the enhancer region; Seq ID No. 36, which includes approximately 17.6 Kb downstream of lambda; Seq ID No. 37, which includes approximately 19.1 Kb downstream of lambda; Seq ID No. 38, which includes approximately 21.3 Kb downstream of lambda; and Seq ID No. 39, which includes approximately 27 Kb downstream of lambda, or any contiguous sequence (such as about 17, 20, 30, 40, 50, 75, 100, 200, 300 or 5000 nucleotides of contiguous sequence) or fragment thereof of Seq ID Nos 32-39 (see also, for example FIG. 6). It is understood that in general when designing a targeting construct one targeting arm will be 5′ of the other targeting arm.

In additional embodiments, the targeting constructs for the lambda locus can contain site specific recombinase sites, such as, for example, lox. In one embodiment, the targeting arms can insert thesite specific recombinase site into the targeted region. Then, the site specific recombinase can be activated and/or applied to the cell such that the intervening nucleotide sequence between the two site specific recombinase sites is excised (see, for example, FIG. 6).

Selectable Marker Genes

The DNA constructs can be designed to modify the endogenous, target immunoglobulin gene. The homologous sequence for targeting the construct can have one or more deletions, insertions, substitutions or combinations thereof. The alteration can be the insertion of a selectable marker gene fused in reading frame with the upstream sequence of the target gene.

Suitable selectable marker genes include, but are not limited to: genes conferring the ability to grow on certain media substrates, such as the tk gene (thymidine kinase) or the hprt gene (hypoxanthine phosphoribosyltransferase) which confer the ability to grow on HAT medium (hypoxanthine, aminopterin and thymidine); the bacterial gpt gene (guanine/xanthine phosphoribosyltransferase) which allows growth on MAX medium (mycophenolic acid, adenine, and xanthine). See, for example, Song, K-Y., et al. Proc. Nat'l Acad. Sci. U.S.A. 84:6820-6824 (1987); Sambrook, J., et al., Molecular Cloning—A Laboratory Manual, Cold Spring Harbor Laboratory, Cold Spring Harbor, N.Y. (1989), Chapter 16. Other examples of selectable markers include: genes conferring resistance to compounds such as antibiotics, genes conferring the ability to grow on selected substrates, genes encoding proteins that produce detectable signals such as luminescence, such as green fluorescent protein, enhanced green fluorescent protein (eGFP). A wide variety of such markers are known and available, including, for example, antibiotic resistance genes such as the neomycin resistance gene (neo) (Southern, P., and P. Berg, J. Mol. Appl. Genet. 1:327-341 (1982)); and the hygromycin resistance gene (hyg) (Nucleic Acids Research 11:6895-6911 (1983), and Te Riele, H., et al., Nature 348:649-651 (1990)). Other selectable marker genes include: acetohydroxyacid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline.

Methods for the incorporation of antibiotic resistance genes and negative selection factors will be familiar to those of ordinary skill in the art (see, e.g., WO 99/15650; U.S. Pat. No. 6,080,576; U.S. Pat. No. 6,136,566; Niwa et al., J. Biochem. 113:343-349 (1993); and Yoshida et al., Transgenic Research 4:277-287 (1995)).

Combinations of selectable markers can also be used. For example, to target an immunoglobulin gene, a neo gene (with or without its own promoter, as discussed above) can be cloned into a DNA sequence which is homologous to the immunoglobulin gene. To use a combination of markers, the HSV-tk gene can be cloned such that it is outside of the targeting DNA (another selectable marker could be placed on the opposite flank, if desired). After introducing the DNA construct into the cells to be targeted, the cells can be selected on the appropriate antibiotics. In this particular example, those cells which are resistant to G418 and gancyclovir are most likely to have arisen by homologous recombination in which the neo gene has been recombined into the immunoglobulin gene but the tk gene has been lost because it was located outside the region of the double crossover.

Deletions can be at least about 50 bp, more usually at least about 100 bp, and generally not more than about 20 kbp, where the deletion can normally include at least a portion of the coding region including a portion of or one or more exons, a portion of or one or more introns, and can or can not include a portion of the flanking non-coding regions, particularly the 5′-non-coding region (transcriptional regulatory region). Thus, the homologous region can extend beyond the coding region into the 5′-non-coding region or alternatively into the 3′-non-coding region. Insertions can generally not exceed 10 kbp, usually not exceed 5 kbp, generally being at least 50 bp, more usually at least 200 bp.

The region(s) of homology can include mutations, where mutations can further inactivate the target gene, in providing for a frame shift, or changing a key amino acid, or the mutation can correct a dysfunctional allele, etc. The mutation can be a subtle change, not exceeding about 5% of the homologous flanking sequences. Where mutation of a gene is desired, the marker gene can be inserted into an intron or an exon.

The construct can be prepared in accordance with methods known in the art, various fragments can be brought together, introduced into appropriate vectors, cloned, analyzed and then manipulated further until the desired construct has been achieved. Various modifications can be made to the sequence, to allow for restriction analysis, excision, identification of probes, etc. Silent mutations can be introduced, as desired. At various stages, restriction analysis, sequencing, amplification with the polymerase chain reaction, primer repair, in vitro mutagenesis, etc. can be employed.

The construct can be prepared using a bacterial vector, including a prokaryotic replication system, e.g. an origin recognizable by E. coli, at each stage the construct can be cloned and analyzed. A marker, the same as or different from the marker to be used for insertion, can be employed, which can be removed prior to introduction into the target cell. Once the vector containing the construct has been completed, it can be further manipulated, such as by deletion of the bacterial sequences, linearization, introducing a short deletion in the homologous sequence. After final manipulation, the construct can be introduced into the cell.

The present invention further includes recombinant constructs containing sequences of immunoglobulin genes. The constructs comprise a vector, such as a plasmid or viral vector, into which a sequence of the invention has been inserted, in a forward or reverse orientation. The construct can also include regulatory sequences, including, for example, a promoter, operably linked to the sequence. Large numbers of suitable vectors and promoters are known to those of skill in the art, and are commercially available. The following vectors are provided by way of example. Bacterial: pBs, pQE-9 (Qiagen), phagescript, PsiX174, pBluescript SK, pBsKS, pNH8a, pNH16a, pNH18a, pNH46a (Stratagene); pTrc99A, pKK223-3, pKK233-3, pDR540, pRIT5 (Pharmacia). Eukaryotic: pWLneo, pSv2cat, pOG44, pXTl, pSG (Stratagene) pSVK3, pBPv, pMSG, pSVL (Pharmiacia), viral origin vectors (M13 vectors, bacterial phage 1 vectors, adenovirus vectors, and retrovirus vectors), high, low and adjustable copy number vectors, vectors which have compatible replicons for use in combination in a single host (pACYC184 and pBR322) and eukaryotic episomal replication vectors (pCDM8). Other vectors include prokaryotic expression vectors such as pcDNA II, pSL301, pSE280, pSE380, pSE420, pTrcHisA, B, and C, pRSET A, B, and C (Invitrogen, Corp.), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and pProEx-HT (Invitrogen, Corp.) and variants and derivatives thereof. Other vectors include eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMC1neo, and pOG44 (Stratagene, Inc.), and pYES2, pAC360, pBlueBacHis A, B, and C, pVL1392, pBlueBacIII, pCDM8, pcDNA1, pZeoSV, pcDNA3 pREP4, pCEP4, and pEBVHis (Invitrogen, Corp.) and variants or derivatives thereof. Additional vectors that can be used include: pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen), pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pPICZ□, pGAPZ, pGAPZ□, pBlueBac4.5, pBlueBacHis2, pMelBac, pSinRep5, pSinHis, pIND, pIND(SP1), pVgRXR, pcDNA2.1, pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; □ ExCell, □ gt11, pTrc99A, pKK223-3, pGEX-1 □T, pGEX-2T, pGEX-2TK, pGEX-4T-1, pGEX-4T-2, pGEX-4T-3, pGEX-3X, pGEX-5X-1, pGEX-SX-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4abc(+), pOCUS-2, pTAg, pET-32LIC, pET-30LIC, pBAC-2cp LIC, pBACgus-2cp LIC, pT7Blue-2 LIC, pT7Blue-2, □SCREEN-1, □BlueSTAR, pET-3abcd, pET-7abc, pET9abcd, pET11abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3cp, pBACgus-2cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, p□gal-Basic, p□gal-Control, p□gal-Promoter, p□gal-Enhancer, pCMV□, pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, □gt10, □gt11, pWE15, and □TriplEx from Clontech; Lambda ZAP II, pBK-CMV, pBK-RSV, pBluescript II KS ±, pBluescript II SK ±, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS ±, pBC KS ±, pBC SK ±, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOPI3 CAT,pXT1, pSG5, pPbac, pMbac, pMC1neo, pMC1neo Poly A, pOG44, pOG45, pFRT□GAL, pNEO□GAL, pRS403, pRS404, pRS405, pRS406, pRS413, pRS414, pRS415, and pRS416 from Stratagene and variants or derivatives thereof Two-hybrid and reverse two-hybrid vectors can also be used, for example, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GALI, pHISi, pHISi-1, placZi, pB42AD, pDG202, pJK202, pJG4-5, pNLexA, pYESTrp and variants or derivatives thereof. Any other plasmids and vectors may be used as long as they are replicable and viable in the host.

Techniques which can be used to allow the DNA construct entry into the host cell include, for example, calcium phosphate/DNA co precipitation, microinjection of DNA into the nucleus, electroporation, bacterial protoplast fusion with intact cells, transfection, or any other technique known by one skilled in the art. The DNA can be single or double stranded, linear or circular, relaxed or supercoiled DNA. For various techniques for transfecting mammalian cells, see, for example, Keown et al., Methods in Enzymology Vol.185, pp. 527-537 (1990).

In one specific embodiment, heterozygous or homozygous knockout cells can be produced by transfection of primary fetal fibroblasts with a knockout vector containing immunoglobulin gene sequence isolated from isogenic DNA. In another embodiment, the vector can incorporate a promoter trap strategy, using, for example, IRES (internal ribosome entry site) to initiate translation of the Neor gene.

Site Specific Recombinases

In additional embodiments, the targeting constructs can contain site specific recombinase sites, such as, for example, lox. In one embodiment, the targeting arms can insert thesite specific recombinase target sites into the targeted region such that one site specific recombinase target site is located 5′ to the second site specific recombinase target site . Then, the site specific recombinase can be activated and/or applied to the cell such that the intervening nucleotide sequence between the two site specific recombinase sites is excised.

Site-specific recombinases include enzymes or recombinases that recognize and bind to a short nucleic acid site or sequence-specific recombinase target site, i.e., a recombinase recognition site, and catalyze the recombination of nucleic acid in relation to these sites. These enzymes include recombinases, transposases and integrases. Examples of sequence-specific recombinase target sites include, but are not limited to, lox sites, att sites, dif sites and frt sites. Non-limiting examples of site-specific recombinases include, but are not limited to, bacteriophage P1 Cre recombinase, yeast FLP recombinase, Inti integrase, bacteriophage λ, phi 80, P22, P2, 186, and P4 recombinase, Tn3 resolvase, the Hin recombinase, and the Cin recombinase, E. coli xerC and xerD recombinases, Bacillus thuringiensis recombinase, TpnI and the β-lactamase transposons, and the immunoglobulin recombinases.

In one embodiment, the recombination site can be a lox site that is recognized by the Cre recombinase of bacteriophage P1. Lox sites refer to a nucleotide sequence at which the product of the cre gene of bacteriophage P1, the Cre recombinase, can catalyze a site-specific recombination event. A variety of lox sites are known in the art, including the naturally occurring loxP, loxB, loxL and loxR, as well as a number of mutant, or variant, lox sites, such as loxP511, loxP514, lox.DELTA.86, lox.DELTA.117, loxC2, loxP2, loxP3 and lox P23. Additional example of lox sites include, but are not limited to, loxB, loxL, loxR, loxP, loxP3, loxP23, loxΔ86, loxΔ117, loxP5 11, and loxC2.

In another embodiment, the recombination site is a recombination site that is recognized by a recombinases other than Cre. In one embodiment, the recombinase site can be the FRT sites recognized by FLP recombinase of the 2 pi plasmid of Saccharomyces cerevisiae. FRT sites refer to a nucleotide sequence at which the product of the FLP gene of the yeast 2 micron plasmid, FLP recombinase, can catalyze site-specific recombination. Additional examples of the non-Cre recombinases include, but are not limited to, site-specific recombinases include: att sites recognized by the Int recombinase of bacteriophage λ (e.g. att1, att2, att3, attP, attB, attL, and attR), the recombination sites recognized by the resolvase family, and the recombination site recognized by transposase of Bacillus thruingiensis.

In particular embodiments of the present invention, the targeting constructs can contain: sequence homologous to a porcine immunoglobulin gene as described herein, a selectable marker gene and/or a site specific recombinase target site.

Selection of Homologously Recombined Cells

The cells can then be grown in appropriately-selected medium to identify cells providing the appropriate integration. The presence of the selectable marker gene inserted into the immunoglobulin gene establishes the integration of the target construct into the host genome. Those cells which show the desired phenotype can then be further analyzed by restriction analysis, electrophoresis, Southern analysis, polymerase chain reaction, etc to analyze the DNA in order to establish whether homologous or non-homologous recombination occurred. This can be determined by employing probes for the insert and then sequencing the 5′ and. 3′ regions flanking the insert for the presence of the immunoglobulin gene extending beyond the flanking regions of the construct or identifying the presence of a deletion, when such deletion is introduced. Primers can also be used which are complementary to a sequence within the construct and complementary to a sequence outside the construct and at the target locus. In this way, one can only obtain DNA duplexes having both of the primers present in the. complementary chains if homologous recombination has occurred. By demonstrating the presence of the primer sequences or the expected size sequence, the occurrence of homologous recombination is supported.

The polymerase chain reaction used for screening homologous recombination events is known in the art, see, for example, Kim and Smithies, Nucleic Acids Res. 16:8887-8903, 1988; and Joyner et al., Nature 338:153-156, 1989. The specific combination of a mutant polyoma enhancer and a thymidine kinase promoter to drive the neomycin gene has been shown to be active in both embryonic stem cells and EC cells by Thomas and Capecchi, supra, 1987; Nicholas and Berg (1983) in Teratocarcinoma Stem Cell, eds. Siver, Martin and Strikland (Cold Spring Harbor Lab., Cold Spring Harbor, N.Y. (pp. 469-497); and Linney and Donerly, Cell 35:693-699, 1983.

The cell lines obtained from the first round of targeting are likely to be heterozygous for the targeted allele. Homozygosity, in which both alleles are modified, can be achieved in a number of ways. One approach is to grow up a number of cells in which one copy has been modified and then to subject these cells to another round of targeting using a different selectable marker. Alternatively, homozygotes can be obtained by breeding animals heterozygous for the modified allele, according to traditional Mendelian genetics. In some situations, it can be desirable to have two different modified alleles. This can be achieved by successive rounds of gene targeting or by breeding heterozygotes, each of which carries one of the desired modified alleles.

Identification of Cells That Have Undergone Homologous Recombination

In one embodiment, the selection method can detect the depletion of the immunoglobulin gene directly, whether due to targeted knockout of the immunoglobulin gene by homologous recombination, or a mutation in the gene that results in a nonfunctioning or nonexpressed immunoglobulin. Selection via antibiotic resistance has been used most commonly for screening (see above). This method can detect the presence of the resistance gene on the targeting vector, but does not directly indicate whether integration was a targeted recombination event or a random integration. Certain technology, such as Poly A and promoter trap technology, increase the probability of targeted events, but again, do not give direct evidence that the desired phenotype, a cell deficient in immunoglobulin gene expression, has been achieved. In addition, negative forms of selection can be used to select for targeted integration; in these cases, the gene for a factor lethal to the cells is inserted in such a way that only targeted events allow the cell to avoid death. Cells selected by these methods can then be assayed for gene disruption, vector integration and, finally, immunoglobulin gene depletion. In these cases, since the selection is based on detection of targeting vector integration and not at the altered phenotype, only targeted knockouts, not point mutations, gene rearrangements or truncations or other such modifications can be detected.

Animal cells believed to lacking expression of functional immunoglobulin genes can be further characterized. Such characterization can be accomplished by the following techniques, including, but not limited to: PCR analysis, Southern blot analysis, Northern blot analysis, specific lectin binding assays, and/or sequencing analysis.

PCR-analysis as described in the art can be used to determine the integration of targeting vectors. In one embodiment, amplimers can originate in the antibiotic resistance gene and extend into a region outside the vector sequence. Southern analysis can also be used to characterize gross modifications in the locus, such as the integration of a targeting vector into the immunoglobulin locus. Whereas, Northern analysis can be used to characterize the transcript produced from each of the alleles.

Further, sequencing analysis of the cDNA produced from the RNA transcript can also be used to determine the precise location of any mutations in the immunoglobulin allele.

In another aspect of the present invention, ungulate cells lacking at least one allele of a functional region of an ungulate heavy chain, kappa light chain and/or lambda light chain locus produced according to the process, sequences and/or constructs described herein are provided. These cells can be obtained as a result of homologous recombination. Particularly, by inactivating at least one allele of an ungulate heavy chain, kappa light chain or lambda light chain gene, cells can be produced which have reduced capability for expression of porcine antibodies. In other embodiments, mammalian cells lacking both alleles of an ungulate heavy chain, kappa light chain and/or lambda light chain gene can be produced according to the process, sequences and/or constructs described herein. In a further embodiment, porcine animals are provided in which at least one allele of an ungulate heavy chain, kappa light chain and/or lambda light chain gene is inactivated via a genetic targeting event produced according to the process, sequences and/or constructs described herein. In another aspect of the present invention, porcine animals are provided in which both alleles of an ungulate heavy chain, kappa light chain and/or lambda light chain gene are inactivated via a genetic targeting event. The gene can be targeted via homologous recombination. In other embodiments, the gene can be disrupted, i.e. a portion of the genetic code can be altered, thereby affecting transcription and/or translation of that segment of the gene. For example, disruption of a gene can occur through substitution, deletion (“knock-out”) or insertion (“knock-in”) techniques. Additional genes for a desired protein or regulatory sequence that modulate transcription of an existing sequence can be inserted.

In embodiments of the present invention, alleles of ungulate heavy chain, kappa light chain or lambda light chain gene are rendered inactive according to the process, sequences and/or constructs described herein, such that functional ungulate immunoglobulins can no longer be produced. In one embodiment, the targeted immunoglobulin gene can be transcribed into RNA, but not translated into protein. In another embodiment, the targeted immunoglobulin gene can be transcribed in an inactive truncated form. Such a truncated RNA may either not be translated or can be translated into a nonfunctional protein. In an alternative embodiment, the targeted immunoglobulin gene can be inactivated in such a way that no transcription of the gene occurs. In a further embodiment, the targeted immunoglobulin gene can be transcribed and then translated into a nonfunctional protein.

III. Insertion of Artificial Chromosomes Containing Human Immunoglobulin Genes

Artificial Chromosomes

One aspect of the present invention provides ungulates and ungulate cells that lack at least one allele of a functional region of an ungulate heavy chain, kappa light chain and/or lambda light chain locus produced according to the processes, sequences and/or constructs described herein, which are further modified to express at least part of a human antibody (i.e. immunoglobulin (Ig)) locus. This human locus can undergoe rearrangement and express a diverse population of human antibody molecules in the ungulate. These cloned, transgenic ungulates provide a replenishable, theoretically infinite supply of human antibodies (such as polyclonal antibodies), which can be used for therapeutic, diagnostic, purification, and other clinically relevant purposes.

In one particular embodiment, artificial chromosome (ACs) can be used to accomplish the transfer of human immunoglobulin genes into ungulate cells and animals. ACs permit targeted integration of megabase size DNA fragments that contain single or multiple genes. The ACs, therefore, can introduce heterologous DNA into selected cells for production of the gene product encoded by the heterologous DNA. In a one embodiment, one or more ACs with integrated human immunoglobulin DNA can be used as a vector for introduction of human Ig genes into ungulates (such as pigs).

First constructed in yeast in 1983, ACs are man-made linear DNA molecules constructed from essential cis-acting DNA sequence elements that are responsible for the proper replication and partitioning of natural chromosomes (Murray et al. (1983), Nature 301:189-193). A chromosome requires at least three elements to function. Specifically, the elements of an artificial chromosome include at least: (1) autonomous replication sequences (ARS) (having properties of replication origins—which are the sites for initiation of DNA replication), (2) centromeres (site of kinetochore assembly that is responsible for proper distribution of replicated chromosomes at mitosis and meiosis), and (3) telomeres (specialized structures at the ends of linear chromosomes that function to both stabilize the ends and facilitate the complete replication of the extreme termini of the DNA molecule).

In one embodiment, the human Ig can be maintained as an independent unit (an episome) apart from the ungulate chromosomal DNA. For example, episomal vectors contain the necessary DNA sequence elements required for DNA replication and maintenance of the vector within the cell. Episomal vectors are available commercially (see, for example, Maniatis, T. et al., Molecular Cloning, A Laboratory Manual (1982) pp. 368-369). The AC can stably replicate and segregate along side endogenous chromosomes. In an alternative embodiment, the human IgG DNA sequences can be integrated into the ungulate cell's chromosomes thereby permitting the new information to be replicated and partitioned to the cell's progeny as a part of the natural chromosomes (see, for example, Wigler et al. (1977), Cell 11:223). The AC can be translocated to, or inserted into, the endogenous chromosome of the ungulate cell. Two or more ACs can be introduced to the host cell simultaneously or sequentially.

ACs, furthermore, can provide an extra-genomic locus for targeted integration of megabase size DNA fragments that contain single or multiple genes, including multiple copies of a single gene operatively linked to one promoter or each copy or several copies linked to separate promoters. ACs can permit the targeted integration of megabase size DNA fragments that contain single or multiple human immunoglobulin genes. The ACs can be generated by culturing the cells with dicentric chromosomes (i.e., chromosomes with two centromeres) under such conditions known to one skilled in the art whereby the chromosome breaks to form a minichromosome and formerly dicentric chromosome.

ACs can be constructed from humans (human artificial chromosomes: “HACs”), yeast (yeast artificial chromosomes: “YACs”), bacteria (bacterial artificial chromosomes: “BACs”), bacteriophage P1-derived artificial chromosomes: “PACs”) and other mammals (mammalian artificial chromosomes: “MACs”). The ACs derive their name (e.g., YAC, BAC, PAC, MAC, HAC) based on the origin of the centromere. A YAC, for example, can derive its centromere from S. cerevisiae. MACs, on the other hand, include an active mammalian centromere while HACs refer to chromosomes that include human centromeres. Furthermore, plant artificial chromosomes (“PLACs”) and insect artificial chromosomes can also be constructed. The ACs can include elements derived from chromosomes that are responsible for both replication and maintenance. ACs, therefore, are capable of stably maintaining large genomic DNA fragments such as human Ig DNA.

In one emobidment, ungulates containing YACs are provided. YACs are genetically engineered circular chromosomes that contain elements from yeast chromosomes, such as S. cerevisiae, and segments of foreign DNAs that can be much larger than those accepted by conventional cloning vectors (e.g., plasmids, cosmids). YACs allow the propagation of very large segments of exogenous DNA (Schlessinger, D. (1990), Trends in Genetics 6:248-253) into mammalian cells and animals (Choi et al. (1993), Nature Gen 4:117-123). YAC transgenic approaches are very powerful and are greatly enhanced by the ability to efficiently manipulate the cloned DNA. A major technical advantage of yeast is the ease with which specific genome modifications can be made via DNA-mediated transformation and homologous recombination (Ramsay, M. (1994), Mol Biotech 1:181-201). In one embodiment, one or more YACs with integrated human Ig DNA can be used as a vector for introduction of human Ig genes into ungulates (such as pigs).

The YAC vectors contain specific structural components for replication in yeast, including: a centromere, telomeres, autonomous replication sequence (ARS), yeast selectable markers (e.g., TRP1, URA3, and SUP4), and a cloning site for insertion of large segments of greater than 50 kb of exogenous DNA. The marker genes can allow selection of the cells carrying the YAC and serve as sites for the synthesis of specific restriction endonucleases. For example, the TRP1 and URA3 genes can be used as dual selectable markers to ensure that only complete artificial chromosomes are maintained. Yeast selectable markers can be carried on both sides of the centromere, and two sequences that seed telomere formation in vivo are separated. Only a fraction of one percent of a yeast cell's total DNA is necessary for replication, however, including the center of the chromosome (the centromere, which serves as the site of attachment between sister chromatids and the sites of spindle fiber attachment during mitosis), the ends of the chromosome (telomeres, which serve as necessary sequences to maintain the ends of eukaryotic chromosomes), and another short stretch of DNA called the ARS which serves as DNA segments where the double helix can unwind and begin to copy itself.

In one embodiment, YACs can be used to clone up to about 1, 2, or 3 Mb of immunoglobulin DNA. In another embodiment, at least 25, 30, 40, 50, 60, 70, 75, 80, 85, 90, or 95 kilobases.

Yeast integrating plasmids, replicating vectors (which are fragments of YACs),can also be used to express human Ig. The yeast integrating plasmid can contain bacterial plasmid sequences that provide a replication origin and a drug-resistance gene for growth in bacteria (e.g., E. coli), a yeast marker gene for selection of transformants in yeast, and restriction sites for inserting Ig sequences. Host cells can stably acquire this plasmid by integrating it directly into a chromosome. Yeast replicating vectors can also be used to express human Ig as free plasmid circles in yeast. Yeast or ARS-containing vectors can be stabilized by the addition of a centromere sequence. YACs have both centromeric and telomeric regions, and can be used for cloning very large pieces of DNA because the recombinant is maintained essentially as a yeast chromosome.

YACs are provided, for example, as disclosed in U.S. Pat. Nos. 6,692,954, 6,495,318, 6,391,642, 6,287,853, 6,221,588, 6,166,288, 6,096,878, 6,015,708, 5,981,175, 5,939,255, 5,843,671, 5,783,385, 5,776,745, 5,578,461, and 4,889,806; European Patent Nos. 1 356 062 and. 0 648 265; PCT Publication Nos. WO 03/025222, WO 02/057437, WO 02/101044, WO 02/057437, WO 98/36082, WO 98/12335, WO 98/01573, WO 96/01276, WO 95/14769, WO 95/05847, WO 94/23049, and WO 94/00569.

In another embodiment, ungulates containing BACs are provided. BACs are F-based plasmids found in bacteria, such as E. Coli, that can transfer approximately 300 kb of foreign DNA into a host cell. Once the Ig DNA has been cloned into the host cell, the newly inserted segment can be replicated along with the rest of the plasmid. As a result, billions of copies of the foreign DNA can be made in a very short time. In a particular embodiment, one or more BACs with integrated human Ig DNA are used as a vector for introduction of human Ig genes into ungulates (such as pigs).

The BAC cloning system is based on the E. coli F-factor, whose replication is strictly controlled and thus ensures stable maintenance of large constructs (Willets, N., and R. Skurray (1987), Structure and function of the F-factor and mechanism of conjugation. In Escherichia coli and Salmonella Typhimurium: Cellular and Molecular Biology (F. C. Neidhardt, Ed) Vol.2 pp 1110-1133, Am. Soc. Microbiol., Washington, D.C.). BACs have been widely used for cloning of DNA from various eukaryotic species (Cai et al. (1995), Genomics 29:413-425; Kim et al. (1996), Genomics 34:213-218; Misumi et al. (1997), Genomics 40:147-150; Woo et al. (1994), Nucleic Acids Res 22:4922-4931; Zimmer, R. and Gibbins, A. M. V. (1997), Genomics 42:217-226). The low occurance of the F-plasmid can reduce the potential for recombination between DNA fragments and can avoid the lethal overexpression of cloned bacterial genes. BACs can stably maintain the human immunoglobulin genes in a single copy vector in the host cells, even after 100 or more generations of serial growth.

BAC (or pBAC) vectors can accommodate inserts in the range of approximately 30 to 300 kb pairs. One specific type of BAC vector, pBeloBacl 1, uses a complementation of the lacZ gene to distinguish insert-containing recombinant molecules from colonies carrying the BAC vector, by color. When a DNA fragment is cloned into the lacZ gene of pBeloBacl 1, insertional activation results in a white colony on X-Gal/IPTG plates after transformation (Kim et al. (1996), Genomics 34:213-218) to easily identify positive clones.

For example, BACs can be provided such as disclosed in U.S. Pat. Nos. 6,713,281, 6,703,198, 6,649,347, 6,638,722, 6,586,184, 6,573,090, 6,548,256, 6,534,262, 6,492,577, 6,492,506, 6,485,912, 6,472,177, 6,455,254, 6,383,756, 6,277,621, 6,183,957, 6,156,574, 6,127,171, 5,874,259, 5,707,811, and 5,597,694; European Patent Nos. 0 805 851; PCT Publication Nos. WO 03/087330, WO 02/00916, WO 01/39797, WO 01/04302, WO 00/79001, WO 99/54487, WO 99/27118, and WO 96/21725.

In another embodiment, ungulates containing bacteriophage PACs are provided. In a particular embodiment, one or more bacteriophage PACs with integrated human Ig DNA are used as a vector for introduction of human Ig genes into ungulates (such as pigs). For example, PACs can be provided such as disclosed in U.S. Pat. Nos. 6,743,906, 6,730,500, 6,689,606, 6,673,909, 6,642,207, 6,632,934, 6,573,090, 6,544,768, 6,489,458, 6,485,912, 6,469,144, 6,462,176, 6,413,776, 6,399,312, 6,340,595, 6,287,854, 6,284,882, 6,277,621, 6,271,008, 6,187,533, 6,156,574, 6,153,740, 6,143,949, 6,017,755, and 5,973,133; European Patent Nos. 0 814 156; PCT Publication Nos. WO 03/091426, WO 03/076573, WO 03/020898, WO 02/101022, WO 02/070696, WO 02/061073, WO 02/31202, WO 01/44486, WO 01/07478, WO 01/05962, and WO 99/63103,.

In a further embodiment, ungulates containing MACs are provided. MACs possess high mitotic stability, consistent and regulated gene expression, high cloning capacity, and non-immunogenicity. Mammalian chromosomes can be comprised of a continuous linear strand of DNA ranging in size from approximately 50 to 250 Mb. The DNA construct can further contain one or more sequences necessary for the DNA construct to multiply in yeast cells. The DNA construct can also contain a sequence encoding a selectable marker gene. The DNA construct can be capable of being maintained as a chromosome in a transformed cell with the DNA construct. MACs provide extra-genomic specific integration sites for introduction of genes encoding proteins of interest and permit megabase size DNA integration so that, for example, genes encoding an entire metabolic pathway, a very large gene [e.g., such as the cystic fibrosis (CF) gene (˜600 kb)], or several genes [e.g., a series of antigens for preparation of a multivalent vaccine] can be stably introduced into a cell.

Mammalian artificial chromosomes [MACs] are provided. Also provided are artificial chromosomes for other higher eukaryotic species, such as insects and fish, produced using the MACS are provided herein. Methods for generating and isolating such chromosomes. Methods using the MACs to construct artificial chromosomes from other species, such as insect and fish species are also provided. The artificial chromosomes are fully functional stable chromosomes. Two types of artificial chromosomes are provided. One type, herein referred to as SATACs [satellite artificial chromosomes] are stable heterochromatic chromosomes, and the another type are minichromosomes based on amplification of euchromatin. As used herein, a formerly dicentric chromosome is a chromosome that is produced when a dicentric chromosome fragments and acquires new telomeres so that two chromosomes, each having one of the centromeres, are produced. Each of the fragments can be replicable chromosomes.

Also provided are artificial chromosomes for other higher eukaryotic species, such as insects and fish, produced using the MACS are provided herein. In one embodiment, SATACs [satellite artificial chromosomes] are provided. SATACs are stable heterochromatic chromosomes. In another embodiment, minichromosomes are provided wherein the minichromosomes are based on amplification of euchromatin.

In one embodiment, artificial chromosomes can be generated by culturing the cells with the dicentric chromosomes under conditions whereby the chromosome breaks to form a minichromosome and formerly dicentric chromosome. In one embodiment, the SATACs can be generated from the minichromosome fragment, see, for example, in U.S. Pat. No. 5,288,625. In another embodiment, the SATACs can be generated from the fragment of the formerly dicentric chromosome. The SATACs can be made up of repeating units of short satellite DNA and can be fully heterochromatic. In one embodiment, absent insertion of heterologous or foreign DNA, the SATACs do not contain genetic information. In other embodiments, SATACs of various sizes are provided that are formed by repeated culturing under selective conditions and subcloning of cells that contain chromosomes produced from the formerly dicentric chromosomes. These chromosomes can be based on repeating units 7.5 to 10 Mb in size, or megareplicons. These megareplicaonscan be tandem blocks of satellite DNA flanked by heterologous non-satellite DNA. Amplification can produce a tandem array of identical chromosome segments [each called an amplicon] that contain two inverted megareplicons bordered by heterologous [“foreign”] DNA. Repeated cell fusion, growth on selective medium and/or BrdU [5-bromodeoxyuridine] treatment or other genome destabilizing reagent or agent, such as ionizing radiation, including X-rays, and subcloning can result in cell lines that carry stable heterochromatic or partially heterochromatic chromosomes, including a 150-200 Mb “sausage” chromosome, a 500-1000 Mb gigachromosome, a stable 250-400 Mb megachromosome and various smaller stable chromosomes derived therefrom. These chromosomes are based on these repeating units and can include human immunoglobulin DNA that is expressed. (See also U.S. Pat. No. 6,743,967.

In other embodiments, MACs can be provided, for example, as disclosed in U.S. Pat. Nos. 6,743,967, 6,682,729, 6,569,643, 6,558,902, 6,548,287, 6,410,722, 6,348,353, 6,297,029, 6,265,211, 6,207,648, 6,150,170, 6,150,160, 6,133,503, 6,077,697, 6,025,155, 5,997,881, 5,985,846, 5,981,225, 5,877,159, 5,851,760, and 5,721,118; PCT Publication Nos. WO 04/066945, WO 04/044129, WO 04/035729, WO 04/033668, WO 04/027075, WO 04/016791, WO 04/009788, WO 04/007750, WO 03/083054, WO 03/068910, WO 03/068909, WO 03/064613, WO 03/052050, WO 03/027315, WO 03/023029, WO 03/012126, WO 03/006610, WO 03/000921, WO 02/103032, WO 02/097059, WO 02/096923, WO 02/095003, WO 02/092615, WO 02/081710, WO 02/059330, WO 02/059296, WO 00/18941, WO 97/16533, and WO 96/40965.

In another aspect of the present invention, ungulates and ungulate cells containing HACs are provided. In a particular embodiment, one or more HACs with integrated human Ig DNA are used as a vector for introduction of human Ig genes into ungulates (such as pigs). In a particular embodiment, one or more HACs with integrated human Ig DNA are used to generate ungulates (for example, pigs) by nuclear transfer which express human Igs in response to immunization and which undergo affinity maturation.

Various approaches may be used to produce ungulates that express human antibodies (“human Ig”). These approaches include, for example, the insertion of a HAC containing both heavy and light chain Ig genes into an ungulate or the insertion of human B-cells or B-cell precursors into an ungulate during its fetal stage or after it is born (e.g., an immune deficient or immune suppressed ungulate) (see, for example, WO 01/35735, filed Nov. 17, 2000, U.S. Ser. No. 02/08645, filed Mar. 20, 2002). In either case, both human antibody producing cells and ungulate antibody-producing B-cells may be present in the ungulate. In an ungulate containing a HAC, a single B-cell may produce an antibody that contains a combination of ungulate and human heavy and light chain proteins. In still other embodiments, the total size of the HAC is at least to approximately 1, 2, 3, 4, 5, 6, 7, 8, 9 or 10 Mb.

For example, HACs can be provided such as disclosed in U.S. Pat. Nos. 6,642,207, 6,590,089, 6,566,066, 6,524,799, 6,500,642, 6,485,910, 6,475,752, 6,458,561, 6,455,026, 6,448,041, 6,410,722, 6,358,523, 6,277,621, 6,265,211, 6,146,827, 6,143,566, 6,077,697,. 6,025,155, 6,020,142, and 5,972,649; U.S. Pat. Application No. 2003/0037347; PCT Publication Nos. WO 04/050704, WO 04/044156, WO 04/031385, WO 04/016791, WO 03/101396, WO 03/097812, WO 03/093469, WO 03/091426, WO 03/057923, WO 03/057849, WO 03/027638, WO 03/020898, WO 02/092812, and WO 98/27200.

Additional examples of ACs into which human immunoglobulin sequences can be inserted for use in the invention include, for example, BACs (e.g., pBeloBAC11 or pBAC108L; see, e.g., Shizuya et al. (1992), Proc Natl Acad Sci USA 89(18):8794-8797; Wang et al. (1997), Biotechniques 23(6):992-994), bacteriophage PACs, YACs (see, e.g., Burke (1990), Genet Anal Tech Appl 7(5):94-99), and MACs (see, e.g., Vos (1997), Nat. Biotechnol. 15(12):1257-1259; Ascenzioni et al. (1997), Cancer Lett 118(2):135-142), such as HACs, see also, U.S. Pat. Nos. 6,743,967, 6,716,608, 6,692,954, 6,670,154, 6,642,207, 6,638,722, 6,573,090, 6,492,506, 6,348,353, 6,287,853, 6,277,621, 6,183,957, 6,156,953, 6,133,503, 6,090,584, 6,077,697, 6,025,155, 6,015,708, 5,981,175, 5,874,259, 5,721,118, and 5,270,201; European Patent Nos. 1 437 400, 1 234 024, 1 356 062, 0 959 134, 1 056 878, 0 986 648, 0 648 265, and 0 338 266; PCT Publication Nos. WO 04/013299, WO 01/07478, WO 00/06715, WO 99/43842, WO 99/27118, WO 98/55637, WO 94/00569, and WO 89/09219. Additional examples incluse those AC provided in, for example, PCT Publication No. WO 02/076508, WO 03/093469, WO 02/097059; WO 02/096923; US Publication Nos US 2003/0113917 and US 2003/003435; and U.S. Pat. No. 6,025,155.

In other embodiments of the present invention, ACs transmitted through male gametogenesis in each generation. The AC can be ihntegrating or non-integrating. In one ambodiment, the AC can be transmitted through mitosis in substantially all dividing cells. In another embodiment, the AC can provide for position independent expression of a human immunogloulin nucleic acid sequence. In a particular embodiment, the AC can have a transmittal efficiency of at least 10% through each male and female gametogenesis. In one particular embodiment, the AC can be circular. In another particular embodiment, the non-integrating AC can be that deposited with the Belgian Coordinated Collections of Microorganisms—BCCM on Mar. 27, 2000 under accession number LMBP 5473 CB. In additional embodiments, methods for producing an AC are provided wherein a mitotically stable unit containing an exogenous nucleic acid transmitted through male gametogenesis is identified; and an entry site in the mitotically stable unit allows for the integration of human immunoglobulin genes into the unit.

In other embodiments, ACs are provided that include: a functional centromere, a selectable marker and/or a unique cloning site. Tin other embodiments, the AC can exhibit one or more of the following properties: it can segregate stably as an independent chromosome, immunoglobulin sequences can be inserted in a controlled way and can expressed from the AC, it can be efficiently transmitted through the male and female germline and/or the transgenic animals can bear the chromosome in greater than about 30, 40, 50, 60, 70, 80 or 90% of its cells.

In particular embodiments, the AC can be isolated from fibroblasts (such as any mammalian or human fibroblast) in which it was mitotically stable. After transfer of the AC into hamster cells, a lox (such as loxp) site and a selectable marker site can be inserted. In other embodiments, the AC can maintain mitotic stability, for example, showing a loss of less than about 5, 2, 1, 0.5 or 0.25 percent per mitosis in the absence of selection. See also, US 2003/0064509 and WO 01/77357.

Xenogenous Immunoglobulin Genes

In another aspect of the present invention, transgenic ungulates are provided that expresses a xenogenous immunoglobulin loci or fragment thereof, wherein the immunoglobulin can be expressed from an immunoglobulin locus that is integrated within an endogenous ungulate chromosome. In one embodiment, ungulate cells derived from the transgenic animals are provided. In one embodiment, the xenogenous immunoglobulin locus can be inherited by offspring. In another embodiment, the xenogenous immunoglobulin locus can be inherited through the male germ line by offspring. In still further embodiments, an artificial chromosome (AC) can contain the xenogenous immunoglobulin. In one embodiment, the AC can be a yeast AC or a mammalian AC. In a further embodiment, the xenogenous locus can be a human immunoglobulin locus or fragment thereof In one embodiment, the human immunoglobulin locus can be human chromosome 14, human chromosome 2, and human chromosome 22 or fragments thereof. In another embodiment, the human immunoglobulin locus can include any fragment of a human immunoglobulin that can undergo rearrangement. In a further embodiment, the human immunoglobulin loci can include any fragment of a human immunoglobulin heavy chain and a human immunoglobulin light chain that can undergo rearrangement. In still further. embodiment, the human immunoglobulin loci can include any human immunoglobulin locus or fragment thereof that can produce an antibody upon exposure to an antigen. In a particular embodiment, the exogenous human immunoglobulin can be expressed in B cells to produce xenogenous immunoglobulin in response to exposure to one or more antigens.

In other embodiments, the transgenic ungulate that lacks any expression of functional endogenous immunoglobulins can be further genetically modified to express an xenogenous immunoglobulin loci. In an alternative embodiment, porcine animals are provided that contain an xenogeous immunoglobulin locus. In one embodiment, the xenogeous immunoglobulin loci can be a heavy and/or light chain immunoglobulin or fragment thereof. In another embodiment, the xenogenous immunoglobulin loci can be a kappa chain locus or fragment thereof and/or a lambda chain locus or fragment thereof. In still further embodiments, an artificial chromosome (AC) can contain the xenogenous immunoglobulin. In one embodiment, the AC can be a yeast AC or a mammalian AC. In a further embodiment, the xenogenous locus can be a human immunoglobulin locus or fragment thereof. In one embodiment, the human immunoglobulin locus can be human chromosome 14, human chromosome 2, and human chromosome 22 or fragments thereof. In another embodiment, the human immunoglobulin locus can include any fragment of a human immunoglobulin that can undergo rearrangement. In a further embodiment, the human immunoglobulin loci can include any fragment of a human immunoglobulin heavy chain and a human immunoglobulin light chain that can undergo rearrangement. In still further embodiment, the human immunoglobulin loci can include any human immunoglobulin locus or fragment thereof that can produce an antibody upon exposure to an antigen. In a particular embodiment, the exogenous human immunoglobulin can be expressed in B cells to produce xenogenous immunoglobulin in response to exposure to one or more antigens.

In other embodiments, the transgenic ungulate that lacks any expression of functional endogenous immunoglobulins can be further genetically modified to express an xenogenous immunoglobulin loci. In an alternative embodiment, porcine animals are provided that contain an xenogeous immunoglobulin locus. In one embodiment, the xenogeous immunoglobulin loci can be a heavy and/or light chain immunoglobulin or fragment thereof In another embodiment, the xenogenous immunoglobulin loci can be a kappa chain locus or fragment thereof and/or a lambda chain locus or fragment thereof. In still further embodiments, an artificial chromosome (AC) can contain the xenogenous immunoglobulin. In one embodiment, the AC can be a yeast AC or a mammalian AC. In a further embodiment, the xenogenous locus can be a human immunoglobulin locus or fragment thereof. In one embodiment, the human immunoglobulin locus can be human chromosome 14, human chromosome 2, and human chromosome 22 or fragments thereof. In another embodiment, the human immunoglobulin locus can include any fragment of a human immunoglobulin that can undergo rearrangement. In a further embodiment, the human immunoglobulin loci can include any fragment of a human immunoglobulin heavy chain and a human immunoglobulin light chain that can undergo rearrangement. In still further embodiment, the human immunoglobulin loci can include any human immunoglobulin locus or fragment thereof that can produce an antibody upon exposure to an antigen. In a particular embodiment, the exogenous human immunoglobulin can be expressed in B cells to produce xenogenous immunoglobulin in response to exposure to one or more antigens.

In another embodiment, porcine animals are provided that contain an xenogeous immunoglobulin locus. In one embodiment, the xenogeous immunoglobulin loci can be a heavy and/or light chain immunoglobulin or fragment thereof. In another embodiment, the xenogenous immunoglobulin loci can be a kappa chain locus or fragment thereof and/or a lambda chain locus or fragment thereof. In still further embodiments, an artificial chromosome (AC) can contain the xenogenous immunoglobulin. In one embodiment, the AC can be a yeast AC or a mammalian AC. In a further embodiment, the xenogenous locus can be a human immunoglobulin locus or fragment thereof. In one embodiment, the human immunoglobulin locus can be human chromosome 14, human chromosome 2, and human chromosome 22 or fragments thereof In another embodiment, the human immunoglobulin locus can include any fragment of a human immunoglobulin that can undergo rearrangement. In a further embodiment, the human immunoglobulin loci can include any fragment of a human immunoglobulin heavy chain and a human immunoglobulin light chain that can undergo rearrangement. In still further embodiment, the human immunoglobulin loci can include any human immunoglobulin locus or fragment thereof that can produce an antibody upon exposure to an antigen. In a particular embodiment, the exogenous human immunoglobulin can be expressed in B cells to produce xenogenous immunoglobulin in response to exposure to one or more antigens.

Human immunoglobulin genes, such as the Ig heavy chain gene (human chromosome 414), Ig kappa chain gene (human chromosome #2) and/or the Ig lambda chain gene (chromosome #22) can be inserted into Acs, as described above. In a particular embodiment, any portion of the human heavy, kappa and/or lambda Ig genes can be inserted into ACs. In one embodiment, the nucleic acid can be at least 70, 80, 90, 95, or 99% identical to the corresponding region of a naturally-occurring nucleic acid from a human. In other embodiments, more than one class of human antibody is produced by the ungulate. In various embodiments, more than one different human Ig or antibody is produced by the ungulate. In one embodiment, an AC containing both a human Ig heavy chain gene and Ig light chain gene, such as an automatic human artificial chromosome (“AHAC,” a circular recombinant nucleic acid molecule that is converted to a linear human chromosome in vivo by an endogenously expressed restriction endonuclease) can be introduced. In one embodiment, the human heavy chain loci and the light chain loci are on different chromosome arms (i.e., on different side of the centromere). In one embodiments, the heavy chain can include the mu heavy chain, and the light chain can be a lambda or kappa light chain. The Ig genes can be introduced simultaneously or sequentially in one or more than one ACs.

In particular embodiments, the ungulate or ungulate cell expresses one or more nucleic acids encoding all or part of a human Ig gene which undergoes rearrangement and expresses more than one human Ig molecule, such as a human antibody protein. Thus, the nucleic acid encoding the human Ig chain or antibody is in its unrearranged form (that is, the nucleic acid has not undergone V(D)J recombination). In particular embodiments, all of the nucleic acid segments encoding a V gene segment of an antibody light chain can be separated from all of the nucleic acid segments encoding a J gene segment by one or more nucleotides. In a particular embodiment, all of the nucleic acid segments encoding a V gene segment of an antibody heavy chain can be separated from all of the nucleic acid segments encoding a D gene segment by one or more nucleotides, and/or all of the nucleic acid segments encoding a D gene segment of an antibody heavy chain are separated from all of the nucleic acid segments encoding a J gene segment by one or more nucleotides. Administration of an antigen to a transgenic ungulate containing an unrearranged human Ig gene is followed by the rearrangement of the nucleic acid segments in the human Ig gene locus and the production of human antibodies reactive with the antigen.

In one embodiment, the AC can express a portion or fragment of a human chromocome that contains an immunoglobulin gene. In one embodiment, the AC can express at least 300 or 1300 kb of the human light chain locus, such as described in Davies et al. 1993 Biotechnology 11: 911-914.

In another embodiment, the AC can express a portion of human chromosome 22 that contains at least the λ light-chain locus, including V_{λ} gene segments, J_{λ} gene segments, and the single C_{λ} gene. In another embodiment, the AC can express at least one V_{λ} gene segment, at least one J_{λ} gene segment, and the C_{λ} gene. In other embodiment, ACs can contain portions of the lambda locus, such as described in Popov et al. J Exp Med. 1999 May 17;189(10):1611-20.

In another embodiment, the AC can express a portion of human chromosome 2 that contains at least the κ light-chain locus, including V_{κ} gene segments, J_{κ} gene segments and the single C_{κ} gene. In another embodiment, the AC can express at least one V_{κ} gene segment, at least one J_{κ} gene segment and the C_{κ} gene. In other embodiments, AC containing portions of the kappa light chain locus can be those describe, for example, in Li et al. 2000 J Immunol 164: 812-824 and Li S Proc Natl Acad Sci USA. June 1987;84(12):4229-33. In another embodiment, AC containing approximatelty 1.3 Mb of human kappa locus are provided, such as descibed in Zou et al FASEB J. August 1996;10(10):1227-32.

In further embodiments, the AC can express a portion of human chromosome 14 that contains at least the human heavy-chain locus, including V_{H}, D_{H}, J_{H} and C_{H} gene segments. In another embodiment, the AC can express at least one V_{H} gene segment, at least one D_{H} gene segment, at least one J_{H} gene segment and at least one at least one C_{H} gene segment. In other embodiments, the AC can express at least 85 kb of the human heavy chain locus, such as described in Choi et al. 1993 Nat Gen 4:117-123 and/or Zou et al. 1996 PNAS 96: 14100-14105.

In other embodiments, the AC can express portions of both heavy and light chain loci, such as, at least 220, 170, 800 or 1020 kb, for example, as disclosed in Green et al. 1994 Nat Gen 7:13-22; Mendez et al 1995 Genomics 26: 294-307; Mendez et al. 1997 Nat Gen 15: 146-156; Green et al. 1998 J Exp Med 188: 483-495 and/or Fishwild et al. 1996 Nat Biotech 14: 845-851. In another embodiment, the AC can express megabase amounts of human immunoglobulin, such as described in Nicholson J Immunol. Dec. 15, 1999;163(12):6898-906 and Popov Gene. Oct. 24, 1996;177(1-2):195-201. In addition, in one particular embodiment, MACs derived from human chromosome #14 (comprising the Ig heavy chain gene), human chromosome #2 comprising the Ig kappa chain gene) and human chromosome #22 (comprising the Ig lambda chain gene) can be introduced simultaneously or successively, such as described in US Patent Publication No. 2004/0068760 to Robl et al. In another embodiments, the total size of the MAC is less than or equal to approximately 10, 9, 8, or 7 megabases.

In a particular embodiment, human Vh, human Dh, human Jh segments and human mu segments of human immunoglobulins in germline configuration can be inserted into an AC, such as a YAC, such that the Vh, Dh, Jh and mu DNA segments form a repertoire of immunoglobulins containing portions which correspond to the human DNA segments, for example, as described in U.S. Pat. No. 5,545,807 to the Babraham Insttitute. Such ACs, after insertion into ungulate cells and generation of ungulates can produce heavy chain immunoglobulins. In one embodiment, these immunoglobulins can form functional heavy chain-light chain immunoglobulins. In another embodiment, these immunoglobulins can be expressed in an amount allowing for recovery from suitable cells or body fluids of the ungulate. Such immunglobulins can be inserted into yeast artifical chromosome vectors, such as decribed by Burke, D T, Carle, G F and Olson, M V (1987) “Cloning of large segments of exogenous DNA into yeast by means of artifical chromosome vectors” Science, 236, 806-812, or by introduction of chromosome fragments (such as described by Richer, J and Lo, C W (1989) “Introduction of human DNA into mouse eggs by injection of dissected human chromosome fragments” Science 245, 175-177).

Additional information on specific ACs containing human immunoglobulin genes can be found in, for example, recent reviews by Giraldo & Montoliu (2001) Transgenic Research 10: 83-103 and Peterson (2003) Expert Reviews in Molecular Medicine 5: 1-25.

AC Transfer Methods

The human immunoglobulin genes can be first inserted into ACs and then the human-immunoglobulin-containing ACs can be inserted into the ungulate cells. Alternatively, the ACs can be transferred to an intermediary mammalian cell, such as a CHO cell, prior to insertion into the ungulate call. In one embodiment, the intermediary mammalian cell can also contain and AC and the first AC can be inserted into the AC of the mammalian cell. In particular, a YAC containing human immunoglobulin genes or fragments thereof in a yeast cell can be transferred to a mammalian cell that harbors an MAC. The YAC can be inserted into the MAC. The MAC can then be transferred to an ungulate cell. The human Ig genes can be inserted into ACs by homologous recombination. The resulting AC containing human Ig genes, can then be introduced into ungulate cells. One or more ungulate cells can be selected by techniques described herein or those known in the art, which contain an AC containing a human Ig.

Suitable hosts for introduction of the ACs are provided herein, which include but are not limited to any animal or plant, cell or tissue thereof, including, but not limited to: mammals, birds, reptiles, amphibians, insects, fish, arachnids, tobacco, tomato, wheat, monocots, dicots and algae. In one embodiment, the ACscan be condensed (Marschall et al Gene Ther. September 1999;6(9):1634-7) by any reagent known in the art, including, but not limited to, spermine, spermidine, polyethylenimine, and/or polylysine prior to introduction into cells. The ACs can be introduced by cell fusion or microcell fusion or subsequent to isolation by any method known to those of skill in this art, including but not limited to: direct DNA transfer, electroporation, nuclear transfer, microcell fusion, cell fusion, spheroplast fusion, lipid-mediated transfer, lipofection, liposomes, microprojectile bombardment, microinjection, calcium phosphate precipitation and/or any other suitable method. Other methods for introducing DNA into cells, include nuclear microinjection, electroporation, bacterial protoplast fusion with intact cells. Polycations, such as polybrene and polyornithine, may also be used. For various techniques for transforming mammalian cells, see e.g., Keown et al. Methods in Enzymology (1990) Vol.185, pp. 527-537; and Mansour et al. (1988) Nature 336:348-352.

The ACs can be introduced by direct DNA transformation; microinjection in cells or embryos, protoplast regeneration for plants, electroporation, microprojectile gun and other such methods known to one skilled in the art (see, e.g., Weissbach et al. (1988) Methods for Plant Molecular Biology, Academic Press, N.Y., Section VIII, pp. 421-463; Grierson et al. (1988) Plant Molecular Biology, 2d Ed., Blackie, London, Ch. 7-9; see, also U.S. Pat. Nos. 5,491,075; 5,482,928; and 5,424,409; see, also, e.g., U.S. Pat. No. 5,470,708,).

In particular embodiments, one or more isolated YACs can be used that harbor human I genes. The isolated YACs can be condensed (Marschall et al Gene Ther. September 1999;6(9):1634-7) by any reagent known in the art, including, but not limited to spermine, spermidine, polyethylenimine, and/or polylysine. The condensed YACs can then be transferred to porcine cells by any method known in the art (for example, microinjection, electroporation, lipid mediated transfection, etc). Alternatively, the condensed YAC can be transferred to oocytes via sperm-mediated gene transfer or intracytoplasmic sperm injection (ICSI) mediated gene transfer. In one embodiment, spheroplast fusion can be used to transfer YACs that harbor human Ig genes to porcine cells.

In other embodiments of the invention, the AC containing the human Ig can be inserted into an adult, fetal, or embryonic ungulate cell. Additional examples of ungulate cells include undifferentiated cells, such as embryonic cells (e.g., embryonic stem cells), differentiated or somatic cells, such as epithelial cells, neural cells epidermal cells, keratinocytes, hematopoietic cells, melanocytes, chondrocytes, B-lymphocytes, T-lymphocytes, erythrocytes, macrophages, monocytes, fibroblasts, muscle cells, cells from the female reproductive system, such as a mammary gland, ovarian cumulus, granulosa, or oviductal cell, germ cells, placental cell, or cells derived from any organ, such as the bladder, brain, esophagus, fallopian tube, heart, intestines, gallbladder, kidney, liver, lung, ovaries, pancreas, prostate, spinal cord, spleen, stomach, testes, thymus, thyroid, trachea, ureter, urethra, and uterus or any other cell type described herein.

Site Specific Recombinase Mediated Transfer

In particular embodiments of the present invention, the transfer of ACs containing human immunoglobulin genes to porcine cells, such as those described herein or known in the art, can be accomplished via site specific recombinase mediated transfer. In one particular embodiment, the ACs can be transferred into porcine fibroblast cells. In another particular embodiment, the ACs can be YACs.

In other embodiments of the present invention, the circularized DNA, such as an AC, that contain the site specific recombinase target site can be transferred into a cell line that has a site specific resombinase target site within its genome. In one embodiment, the cell's site specific recombinase target site can be located within an exogenous chromosome. The exogenous chromosome can be an artificial chromosome that does not integrate into the host's endogenous genome. In one embodiment, the AC can be transferred via germ line transmission to offspring. In one particular embodiment, a YAC containing a human immunoglobulin gene or fragment thereof can be circularized via a site specific recombinase and then transferred into a host cell that contains a MAC, wherein the MAC contains a site specific recombinase site. This MAC that now contains human immunoglobulin loci or fragments thereof can then be fused with a porcine cell, such as, but not limited to, a fibroblast. The porcine cell can then be used for nuclear transfer.

In certain embodiments of the present invention, the ACs that contain human immunoglobulin genes or fragments thereof can be transferred to a mammalian cell, such as a CHO cell, prior to insertion into the ungulate call. In one embodiment, the intermediary mammalian cell can also contain and AC and the first AC can be inserted into the AC of the mammalian cell. In particular, a YAC containing human immunoglobulin genes or fragments thereof in a yeast cell can be transferred to a mammalian cell that harbors a MAC. The YAC can be inserted in the MAC. The MAC can then be transferred to an ungulate cell. In particular embodiments, the YAC harboring the human Ig genes or fragments thereof can contain site specific recombinase trarget sites. The YAC can first be circularized via application of the appropriate site specific recombinase and then inserted into a mammalian cell that contains its own site specific recombinase target site. Then, the site specific recombinase can be applied to inegrate the YAC into the MAC in the intermediary mammalian cell. The site specific recoombinase can be applied in cis or trans. In particular, the site specific recombinase can be applied in trans. In one embodiment, the site specific recombinase can be expressed via transfection of a site specific recombainse expression plasmid, such as a Cre expression plasmid. In addition, one telomere region of the YAC can also be retrofitted with a selectable marker, such as a selectable marker described herein or known in the art. The human Ig genes or fragments thereof within the MAC of the intermediary mammalian cell can then be transferred to an ungulate cell, such as a fibroblast.

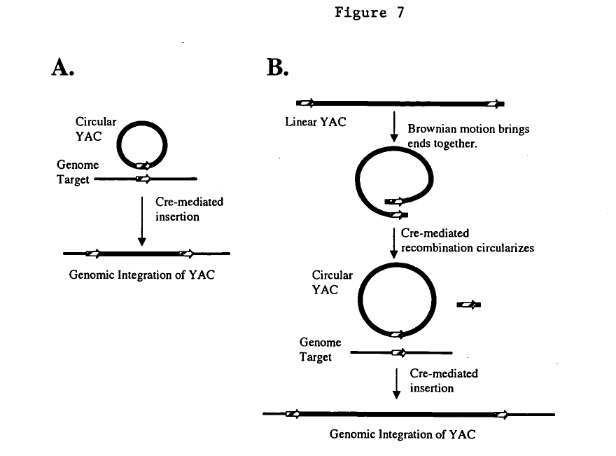

Alternatively, the AC, such as a YAC, harboring the human Ig genes or fragments thereof can contain site specific recombinase target sites optionally located near each telomere. The YAC can first be circularized via application of the appropriate site specific recombinase and then inserted into an ungulate cell directly that contains its own site specific recombinase target site within it genome. Alternatively, the ungulate cell can harbor its own MAC, which contains a site specific recombinase target site. In this embodiment, the YAC can be inserted directly into the endogenous genome of the ungulate cell. In particular embodiments, the ungulate cell can be a fibroblast cell or any other suitable cell that can be used for nuclear transfer. See, for example, FIG. 7; Call et al., Hum Mol Genet. Jul. 22, 2000;9(12):1745-51.

In other embodiments, methods to circularize at least 100 kb of DNA are provided wherein the DNA can then be integrated into a host genome via a site specific recombinase. In one embodiment, at least 100, 200, 300, 400, 500, 1000, 2000, 5000, 10,000 kb of DNA can be circularized. In another embodiment, at least 1000, 2000, 5000, 10,000, or 20,000 megabases of DNA can be circularized. In one embodiment, the circularization of the DNA can be accomplished by attaching site specific recombinase target sites at each end of the DNA sequence and then applying the site specific recombinase to result in circularization of the DNA. In one embodiment, the site specific recombinase target site can be lox. In another embodiment, the site specific recombinase target site can be Flt. In certain embodiments, the DNA can be an artificial chromosome, such as a YAC or any AC described herein or known in the art. In another embodiment, the AC can contain human immunoglobulin loci or fragments thereof.

In another preferred embodiment, the YAC can be converted to, or integrated within, an artificial mammalian chromosome. The mammalian artificial chromosome is either transferred to or harbored within a porcine cell. The artificial chromosome can be introduced within the porcine genome through any method known in the art including but not limited to direct injection of metaphase chromosomes, lipid mediated gene transfer, or microcell fusion.

Site-specific recombinases include enzymes or recombinases that recognize and bind to a short nucleic acid site or sequence-specific recombinase target site, i.e., a recombinase recognition site, and catalyze the recombination of nucleic acid in relation to these sites. These enzymes include recombinases, transposases and integrases. Examples of sequence-specific recombinase target sites include, but are not limited to, lox sites, att sites, dif sites and frt sites. Non-limiting examples of site-specific recombinases include, but are not limited to, bacteriophage P1 Cre recombinase, yeast FLP recombinase, Inti integrase, bacteriophage λ, phi 80, P22, P2, 186, and P4 recombinase, Tn3 resolvase, the Hin recombinase, and the Cin recombinase, E. coli xerC and xerD recombinases, Bacillus thuringiensis recombinase, TpnI and the β-lactamase transposons, and the immunoglobulin recombinases.

In one embodiment, the recombination site can be a lox site that is recognized by the Cre recombinase of bacteriophage P1. Lox sites refer to a nucleotide sequence at which the product of the cre gene of bacteriophage P1, the Cre recombinase, can catalyze a site-specific recombination event. A variety of lox sites are known in the art, including the naturally occurring loxP, loxB, loxL and loxR, as well as a number of mutant, or variant, lox sites, such as loxP511, loxP514, lox.DELTA.86, lox.DELTA.117, loxC2, loxP2, loxP3 and lox P23. Additional example of lox sites include, but are not limited to, loxB, loxL, loxR, loxP, loxP3, loxP23, loxΔ86, loxΔ117, loxP511, and loxC2.

In another embodiment, the recombination site is a recombination site that is recognized by a recombinases other than Cre. In one embodiment, the recombinase site can be the FRT sites recognized by FLP recombinase of the 2 pi plasmid of Saccharomyces cerevisiae. FRT sites refer to a nucleotide sequence at which the product of the FLP gene of the yeast 2 micron plasmid, FLP recombinase, can catalyze site-specific recombination. Additional examples of the non-Cre recombinases include, but are not limited to, site-specific recombinases include: att sites recognized by the Int recombinase of bacteriophage λ (e.g. att1, att2, att3, attP, attB, attL, and attR), the recombination sites recognized by the resolvase family, and the recombination site recognized by transposase of Bacillus thruingiensis.

IV. Production of Genetically Modified Animals

In additional aspects of the present invention, ungulates that contain the genetic modifications described herein can be produced by any method known to one skilled in the art. Such methods include, but -are not limited to: nuclear transfer, intracytoplasmic sperm injection, modification of zygotes directly and sperm mediated gene transfer.

In another embodiment, a method to clone such animals, for example, pigs, includes: enucleating an oocyte, fusing the oocyte with a donor nucleus from a cell in which at least one allele of at least one immunoglobulin gene has been inactivated, and implanting the nuclear transfer-derived embryo into a surrogate mother.

Alternatively, a method is provided for producing viable animals that lack any expression of functional immunoglobulin by inactivating both alleles of the immunoglobulin gene in embryonic stem cells, which can then be used to produce offspring.

In another aspect, the present invention provides a method for producing viable animals, such as pigs, in which both alleles of the immunoglobulin gene have been rendered inactive. In one embodiment, the animals are produced by cloning using a donor nucleus from a cell in which both alleles of the immunoglobulin gene have been inactivated. In one embodiment, both alleles of the immunoglobulin gene are inactivated via a genetic targeting event.

Genetically altered animals that can be created by modifying zygotes directly. For mammals, the modified zygotes can be then introduced into the uterus of a pseudopregnant female capable of carrying the animal to term. For example, if whole animals lacking an immunoglobulin gene are desired, then embryonic stem cells derived from that animal can be targeted and later introduced into blastocysts for growing the modified cells into chimeric animals. For embryonic stem cells, either an embryonic stem cell line or freshly obtained stem cells can be used.

In a suitable embodiment of the invention, the totipotent cells are embryonic stem (ES) cells. The isolation of ES cells from blastocysts, the establishing of ES cell lines and their subsequent cultivation are carried out by conventional methods as described, for example, by Doetchmann et al., J. Embryol. Exp. Morph. 87:27-45 (1985); Li et al., Cell 69:915-926 (1992); Robertson, E. J. “Tetracarcinomas and Embryonic Stem Cells: A Practical Approach,” ed. E. J. Robertson, IRL Press, Oxford, England (1987); Wurst and Joyner, “Gene Targeting: A Practical Approach,” ed. A. L. Joyner, IRL Press, Oxford, England (1993); Hogen et al., “Manipulating the Mouse Embryo: A Laboratory Manual,” eds. Hogan, Beddington, Costantini and Lacy, Cold Spring Harbor Laboratory Press, New York (1994); and Wang et al., Nature 336:741-744 (1992). In another suitable embodiment of the invention, the totipotent cells are embryonic germ (EG) cells. Embryonic Germ cells are undifferentiated cells finctionally equivalent to ES cells, that is they can be cultured and transfected in vitro, then contribute to somatic and germ cell lineages of a chimera (Stewart et al., Dev. Biol. 161:626-628 (1994)). EG cells are derived by culture of primordial germ cells, the progenitors of the gametes, with a combination of growth factors: leukemia inhibitory factor, steel factor and basic fibroblast growth factor (Matsui et al., Cell 70:841-847 (1992); Resnick et al., Nature 359:550-551 (1992)). The cultivation of EG cells can be carried out using methods described in the article by Donovan et al., “Transgenic Animals, Generation and Use,” Ed. L. M. Houdebine, Harwood Academic Publishers (1997), and in the original literature cited therein.

Tetraploid blastocysts for use in the invention may be obtained by natural zygote production and development, or by known methods by electrofusion of two-cell embryos and subsequently cultured as described, for example, by James et al., Genet. Res. Camb. 60:185-194 (1992); Nagy and Rossant, “Gene Targeting: A Practical Approach,” ed. A. L. Joyner, IRL Press, Oxford, England (1993); or by Kubiak and Tarkowski, Exp. Cell Res. 157:561-566 (1985).

The introduction of the ES cells or EG cells into the blastocysts can be carried out by any method known in the art. A suitable method for the purposes of the present invention is the microinjection method as described by Wang et al., EMBO J. 10:2437-2450 (1991).

Alternatively, by modified embryonic stem cells transgenic animals can be produced. The genetically modified embryonic stem cells can be injected into a blastocyst and then brought to term in a female host mammal in accordance with conventional techniques. Heterozygous progeny can then be screened for the presence of the alteration at the site of the target locus, using techniques such as PCR or Southern blotting. After mating with a wild-type host of the same species, the resulting chimeric progeny can then be cross-mated to achieve homozygous hosts.

After transforming embryonic stem cells with the targeting vector to alter the immunoglobulin gene, the cells can be plated onto a feeder layer in an appropriate medium, e.g., fetal bovine serum enhanced DMEM. Cells containing the construct can be detected by employing a selective medium, and after sufficient time for colonies to grow, colonies can be picked and analyzed for the occurrence of homologous recombination. Polymerase chain reaction can be used, with primers within and without the construct sequence but at the target locus. Those colonies which show homologous recombination can then be used for embryo manipulating and blastocyst injection. Blastocysts can be obtained from superovulated females. The embryonic stem cells can then be trypsinized and the modified cells added to a droplet containing the blastocysts. At least one of the modified embryonic stem cells can be injected into the blastocoel of the blastocyst. After injection, at least one of the blastocysts can be returned to each uterine horn of pseudopregnant females. Females are then allowed to go to term and the resulting litters screened for mutant cells having the construct. The blastocysts are selected for different parentage from the transformed ES cells. By providing for a different phenotype of the blastocyst and the ES cells, chimeric progeny can be readily detected, and then genotyping can be conducted to probe for the presence of the modified immunoglobulin gene.

In other embodiments, sperm mediated gene transfer can be used to produce the genetically modified ungulates described herein. The methods and compositions described herein to either eliminate expression of endogenous immunoglobulin genes or insert xenogenous immunoglobulin genes can be used to genetically modify the sperm cells via any technique described herein or known in the art. The genetically modified sperm can then be used to impregnate a female recipient via artificial insemination, intracytoplasmic sperm injection or any other known technique. In one embodiment, the sperm and/or sperm head can be incubated with the exogenous nucleic acid for a sufficient time period. Sufficient time periods include, for. example, about 30 seconds to about 5 minutes, typically about 45 seconds to about 3 minutes, more typically about 1 minute to about 2 minutes. In particular embodiments, the expression of xenogenous, such as human, immunoglobulin genes in ungulates as descrbed herein, can be accomplished via intracytoplasmic sperm injection.

The potential use of sperm cells as vectors for gene transfer was first suggested by Brackett et al., Proc., Natl. Acad. Sci. USA 68:353-357 (1971). This was followed by reports of the production of transgenic mice and pigs after in vitro fertilization of oocytes with sperm that had been incubated by naked DNA (see, for example, Lavitrano et al., Cell 57:717-723 (1989) and Gandolfi et al. Journal of Reproduction and Fertility Abstract Series 4, 10 (1989)), although other laboratories were not able to repeat these experiments (see, for example, Brinster et al. Cell 59:239-241 (1989) and Gavora et al., Canadian Journal of Animal Science 71:287-291 (1991)). Since then, there have been several reports of successful sperm mediated gene transfer in chicken (see, for example, Nakanishi and Iritani, Mol. Reprod. Dev. 36:258-261 (1993)); mice (see, for example, Maione, Mol. Reprod. Dev. 59:406 (1998)); and pigs (see, for example, Lavitrano et al. Transplant. Proc. 29:3508-3509 (1997); Lavitrano et al., Proc. Natl. Acad. Sci. USA 99:14230-5 (2002); Lavitrano et al., Mol. Reprod. Dev. 64-284-91 (2003)). Similar techniques are also described in U.S. Pat. No. 6,376,743; issued Apr. 23, 2002; U.S. Patent Publication Nos. 20010044937, published Nov. 22, 2001, and 20020108132, published Aug. 8, 2002.

In other embodiments, intracytoplasmic sperm injection can be used to produce the genetically modified ungulates described herein. This can be accomplished by coinserting an exogenous nucleic acid and a sperm into the cytoplasm of an unfertilized oocyte to form a transgenic fertilized oocyte, and allowing the transgenic fertilized oocyte to develop into a transgenic embryo and, if desired, into a live offspring. The sperm can be a membrane-disrupted sperm head or a demembranated sperm head. The coinsertion step can include the substep of preincubating the sperm with the exogenous nucleic acid for a sufficient time period, for example, about 30 seconds to about 5 minutes, typically about 45 seconds to about 3 minutes, more typically about 1 minute to about 2 minutes. The coinsertion of the sperm and exogenous nucleic acid into the oocyte can be via microinjection. The exogenous nucleic acid mixed with the sperm can contain more than one transgene, to produce an embryo that is transgenic for more than one transgene as described herein. The intracytoplasmic sperm injection can be accomplished by any technique known in the art, see, for example, U.S. Pat. No. 6,376,743. In particular embodiments, the expression of xenogenous, such as human, immunoglobulin genes in ungulates as descrbed herein, can be accomplished via intracytoplasmic sperm injection.

Any additional technique known in the art may be used to introduce the transgene into animals. Such techniques include, but are not limited to pronuclear microinjection (see, for example, Hoppe, P. C. and Wagner, T. E., 1989, U.S. Pat. No. 4,873,191); retrovirus mediated gene transfer into germ lines (see, for example, Yan der Putten et al., 1985, Proc. Natl. Acad. Sci., USA 82:6148-6152); gene targeting in embryonic stem cells (see, for example, Thompson et al., 1989, Cell 56:313-321; Wheeler, M. B., 1994, WO 94/26884); electroporation of embryos (see, for example, Lo, 1983, Mol Cell. Biol. 3:1803-1814); cell gun; transfection; transduction; retroviral infection; adenoviral infection; adenoviral-associated infection; liposome-mediated gene transfer; naked DNA transfer; and sperm-mediated gene transfer (see, for example, Lavitrano et al., 1989, Cell 57:717-723); etc. For a review of such techniques, see, for example, Gordon, 1989, Transgenic Animals, Intl. Rev. Cytol. 115:171-229. In particular embodiments, the expression of xenogenous, such as human, immunoglobulin genes in ungulates as descrbed herein, can be-accomplished via these techniques.

Somatic Cell Nuclear Transfer to Produce Cloned, Transgenic Offspring

In a further aspect of the present invention, ungulate, such as porcine or bovine, cells lacking one allele, optionally both alleles of an ungulate heavy chain, kappa light chain and/or lambda light chain gene can be used as donor cells for nuclear transfer into recipient cells to produce cloned, transgenic animals. Alternatively, ungulate heavy chain, kappa light chain and/or lambda light chain gene knockouts can be created in embryonic stem cells, which are then used to produce offspring. Offspring lacking a single allele of a functional ungulate heavy chain, kappa light chain and/or lambda light chain gene produced according to the process, sequences and/or constructs described herein can be breed to further produce offspring lacking functionality in both alleles through mendelian type inheritance.

In another embodiment, the present invention provides a method for producing viable pigs that lack any expression of functional alpha-1,3-GT by breeding a male pig heterozygous for the alpha-1,3-GT gene with a female pig heterozygous for the alpha-1,3-GT gene. In one embodiment, the pigs are heterozygous due to the genetic modification of one allele of the alpha-1,3-GT gene to prevent expression of that allele. In another embodiment, the pigs are heterozygous due to the presence of a point mutation in one allele of the alpha-1,3-GT gene. In another embodiment, the point mutation can be a T-to-G point mutation at the second base of exon 9 of the alpha-1,3-GT gene. In one specific embodiment, a method to produce a porcine animal that lacks any expression of functional alpha-1,3-GT is provided wherein a male pig that contains a T-to-G point mutation at the second base of exon 9 of the alpha-1,3-GT gene is bred with a female pig that contains a T-to-G point mutation at the second base of exon 9 of the alpha-1,3-GT gene, or vise versa.

The present invention provides a method for cloning an animal, such as a pig, lacking a functional immunoglobulin gene via somatic cell nuclear transfer. In general, the animal can be produced by a nuclear transfer process comprising the following steps: obtaining desired differentiated cells to be used as a source of donor nuclei; obtaining oocytes from the animal; enucleating said oocytes; transferring the desired differentiated cell or cell nucleus into the enucleated oocyte, e.g., by fusion or injection, to form NT units; activating the resultant NT unit; and transferring said cultured NT unit to a host animal such that the NT unit develops into a fetus.

Nuclear transfer techniques or nuclear transplantation techniques are known in the art(Dai et al. Nature Biotechnology 20:251-255; Polejaeva et al Nature 407:86-90 (2000); Campbell et al, Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci., USA, 90:6143-6147 (1993); WO 94/26884; WO 94/24274, and WO 90/03432, U.S. Pat. Nos. 4,944,384 and 5,057,420).

A donor cell nucleus, which has been modified to alter the immunoglobulin gene, is transferred to a recipient oocyte. The use of this method is not restricted to a particular donor cell type. The donor cell can be as described herein, see also, for example, Wilmut et al Nature 385 810 (1997); Campbell et al Nature 380 64-66 (1996); Dai et al., Nature Biotechnology 20:251-255, 2002 or Cibelli et al Science 280 1256-1258 (1998). All cells of normal karyotype, including embryonic, fetal and adult somatic cells which can be used successfully in nuclear transfer can be employed. Fetal fibroblasts are a particularly useful class of donor cells. Generally suitable methods of nuclear transfer are described in Campbell et al Theriogenology 43 181 (1995), Dai et al. Nature Biotechnology 20:251-255, Polejaeva et al Nature 407:86-90 (2000), Collas et al Mol. Reprod. Dev. 38 264-267 (1994), Keefer et al Biol. Reprod. 50 935-939 (1994), Sims et al Proc. Nat'l. Acad. Sci. USA 90 6143-6147 (1993), WO-A-9426884, WO-A-9424274, WO-A-9807841, WO-A-9003432, U.S. Pat. No. 4,994,384 and U.S. Pat. No. 5,057,420. Differentiated or at least partially differentiated donor cells can also be used. Donor cells can also be, but do not have to be, in culture and can be quiescent. Nuclear donor cells which are quiescent are cells which can be induced to enter quiescence or exist in a quiescent state in vivo. Prior art methods have also used embryonic cell types in cloning procedures (Campbell et al (Nature, 380:64-68, 1996) and Stice et al (Biol. Reprod., 20 54:100-110, 1996).

Somatic nuclear donor cells may be obtained from a variety of different organs and tissues such as, but not limited to, skin, mesenchyme, lung, pancreas, heart, intestine, stomach, bladder, blood vessels, kidney, urethra, reproductive organs, and a disaggregated preparation of a whole or part of an embryo, fetus, or adult animal. In a suitable embodiment of the invention, nuclear donor cells are selected from the group consisting of epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, OxtendedO cells, cumulus cells, epidermal cells or endothelial cells. In another embodiment, the nuclear donor cell is an embryonic stem cell. In a particular embodiment, fibroblast cells can be used as donor cells.

In another embodiment of the invention, the nuclear donor cells of the invention are germ cells of an animal. Any germ cell of an animal species in the embryonic, fetal, or adult stage may be used as a nuclear donor cell. In a suitable embodiment, the nuclear donor cell is an embryonic germ cell.

Nuclear donor cells may be arrested in any phase of the cell cycle (G0, G1, G2, S, M) so as to ensure coordination with the acceptor cell. Any method known in the art may be used to manipulate the cell cycle phase. Methods to control the cell cycle phase include, but are not limited to, G0 quiescence induced by contact inhibition of cultured cells, G0 quiescence induced by removal of serum or other essential nutrient, G0 quiescence induced by senescence, G0 quiescence induced by addition of a specific growth factor; G0 or G1 quiescence induced by physical or chemical means such as heat shock, hyperbaric pressure or other treatment with a chemical, hormone, growth factor or other substance; S-phase control via treatment with a chemical agent which interferes with any point of the replication procedure; M-phase control via selection using fluorescence activated cell sorting, mitotic shake off, treatment with microtubule. disrupting agents or any chemical which disrupts progression in mitosis (see also Freshney, R. I,. “Culture of Animal Cells: A Manual of Basic Technique,” Alan R. Liss, Inc, New York (1983).

Methods for isolation of oocytes are well known in the art. Essentially, this can comprise isolating oocytes from the ovaries or reproductive tract of an animal. A readily available source of oocytes is slaughterhouse materials. For the combination of techniques such as genetic engineering, nuclear transfer and cloning, oocytes must generally be matured in vitro before these cells can be used as recipient cells for nuclear transfer, and before they can be fertilized by the sperm cell to develop into an embryo. This process generally requires collecting immature (prophase I) oocytes from mammalian ovaries, e.g., bovine ovaries obtained at a slaughterhouse, and maturing the oocytes in a maturation medium prior to fertilization or enucleation until the oocyte attains the metaphase II stage, which in the case of bovine oocytes generally occurs about 18-24 hours post-aspiration. This period of time is known as the “maturation period”. In certain embodiments, the oocyte is obtained from a gilt. A “gilt” is a female pig that has never had offspring. In other embodiments, the oocyte is obtained from a sow. A “sow” is a female pig that has previously produced offspring.

A metaphase II stage oocyte can be the recipient oocyte, at this stage it is believed that the oocyte can be or is sufficiently “activated” to treat the introduced nucleus as it does a fertilizing sperm. Metaphase II stage oocytes, which have been matured in vivo have been successfully used in nuclear transfer techniques. Essentially, mature metaphase II oocytes can be collected surgically from either non-superovulated or superovulated animal 35 to 48, or 39-41, hours past the onset of estrus or past the injection of human chorionic gonadotropin (hCG) or similar hormone. The oocyte can be placed in an appropriate medium, such as a hyalurodase solution.

After a fixed time maturation period, which ranges from about 10 to 40 hours, about 16-18 hours, about 40-42 hours or about 39-41 hours, the oocytes can be enucleated. Prior to enucleation the oocytes can be removed and placed in appropriate medium, such as HECM containing 1 milligram per milliliter of hyaluronidase prior to removal of cumulus cells. The stripped oocytes can then be screened for polar bodies, and the selected metaphase II oocytes, as determined by the presence of polar bodies, are then used for nuclear transfer. Enucleation follows.

Enucleation can be performed by known methods, such as described in U.S. Pat. No. 4,994,384. For example, metaphase II oocytes can be placed in either HECM, optionally containing 7.5 micrograms per milliliter cytochalasin B, for immediate enucleation, or can be placed in a suitable medium, for example an embryo culture medium such as CR1aa, plus 10% estrus cow serum, and then enucleated later, such as not more than 24 hours later,or not more than 16-18 hours later.

Enucleation can be accomplished microsurgically using a micropipette to remove the polar body and the adjacent cytoplasm. The oocytes can then be screened to identify those of which have been successfully enucleated. One way to screen the oocytes is to stain the oocytes with 1 microgram per milliliter 33342 Hoechst dye in HECM, and then view the oocytes under ultraviolet irradiation for less than 10 seconds. The oocytes that have been successfully enucleated can then be placed in a suitable culture medium, for example, CR1aa plus 10% serum.

A single mammalian cell of the same species as the enucleated oocyte can then be transferred into the perivitelline space of the enucleated oocyte used to produce the NT unit. The mammalian cell and the enucleated oocyte can be used to produce NT units according to methods known in the art. For example, the cells can be fused by electrofusion. Electrofusion is accomplished by providing a pulse of electricity that is sufficient to cause a transient breakdown of the plasma membrane. This breakdown of the plasma membrane is very short because the membrane reforms rapidly. Thus, if two adjacent membranes are induced to breakdown and upon reformation the lipid bilayers intermingle, small channels can open between the two cells. Due to the thermodynamic instability of such a small opening, it enlarges until the two cells become one. See, for example, U.S. Pat. No. 4,997,384 by Prather et al. A variety of electrofusion media can be used including, for example, sucrose, mannitol, sorbitol and phosphate buffered solution. Fusion can also be accomplished using Sendai virus as a fusogenic agent (Graham, Wister Inot. Symp. Monogr., 9, 19, 1969). Also, the nucleus can be injected directly into the oocyte rather than using electroporation fusion. See, for example, Collas and Barnes, Mol. Reprod. Dev., 38:264-267 (1994). After fusion, the resultant fused NT units are then placed in a suitable medium until activation, for example, CR1aa medium. Typically activation can be effected shortly thereafter, for example less than 24 hours later, or about 4-9 hours later, or optimally 1-2 hours after fusion. In a particular embodiment, activation occurs at least one hour post fusion and at 40-41 hours post maturation.

The NT unit can be activated by known methods. Such methods include, for example, culturing the NT unit at sub-physiological temperature, in essence by applying a cold, or actually cool temperature shock to the NT unit. This can be most conveniently done by culturing the NT unit at room temperature, which is cold relative to the physiological temperature conditions to which embryos are normally exposed. Alternatively, activation can be achieved by application of known activation agents. For example, penetration of oocytes by sperm during fertilization has been shown to activate prefusion oocytes to yield greater numbers of viable pregnancies and multiple genetically identical calves after nuclear transfer. Also, treatments such as electrical and chemical shock can be used to activate NT embryos after fusion. See, for example, U.S. Pat. No. 5,496,720, to Susko-Parrish et al. Fusion and activation can be induced by application of an AC pulse of 5 V for 5 s followed by two DC pulses of 1.5 kV/cm for 60 μs each using an ECM2001 Electrocell Manipulator (BTX Inc., San Diego, Calif.). Additionally, activation can be effected by simultaneously or sequentially by increasing levels of divalent cations in the oocyte, and reducing phosphorylation of cellular proteins in the oocyte. This can generally be effected by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators. Phosphorylation can be reduced by known methods, for example, by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins can be inhibited by introduction of a phosphatase into the oocyte, e.g., phosphatase 2A and phosphatase 2B.

The activated NT units, or “fused embyos”, can then be cultured in a suitable in vitro culture medium until the generation of cell colonies. Culture media suitable for culturing and maturation of embryos are well known in the art. Examples of known media, which can be used for embryo culture and maintenance, include Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media, and, in one specific example, the activated NT units can be cultured in NCSU-23 medium for about 1-4 h at approximately 38.6° C. in a humidified atmosphere of 5% CO2.

Afterward, the cultured NT unit or units can be washed and then placed in a suitable media contained in well plates which can contain a suitable confluent feeder layer. Suitable feeder layers include, by way of example, fibroblasts and epithelial cells. The NT units are cultured on the feeder layer until the NT units reach a size suitable for transferring to a recipient female, or for obtaining cells which can be used to produce cell colonies. These NT units can be cultured until at least about 2 to 400 cells, about 4 to 128 cells, or at least about 50 cells.

Activated NT units can then be transferred (embryo transfers) to the oviduct of an female pigs. In one embodiment, the female pigs can be an estrus-synchronized recipient gilt. Crossbred gilts (large white/Duroc/Landrace) (280-400 lbs) can be used. The gilts can be synchronized as recipient animals by oral administration of 18-20 mg Regu-Mate (Altrenogest, Hoechst, Warren, N.J.) mixed into the feed. Regu-Mate can be fed for 14 consecutive days. One thousand units of Human Chorionic Gonadotropin (hCG, Intervet America, Millsboro, Del.) can then be administered i.m. about 105 h after the last Regu-Mate treatment. Embryo transfers can then be performed about 22-26 h after the hCG injection. In one embodiment, the pregnancy can be brought to term and result in the birth of live offspring. In another embodiment, the pregnancy can be terminated early and embryonic cells can be harvested.

Breeding for Desired Homozygous Knockout Animals

In another aspect, the present invention provides a method for producing viable animals that lack any expression of a functional immunoglobulin gene is provided by breeding a male heterozygous for the immunoglobulin gene with a female heterozygous for the immunoglobulin gene. In one embodiment, the animals are heterozygous due to the genetic modification of one allele of the immunoglobulin gene to prevent expression of that allele. In another embodiment, the animals are heterozygous due to the presence of a point mutation in one allele of the alpha-immunoglobulin gene. In further embodiments, such heterozygous knockouts can be bred with an ungulate that expresses xenogenous immunoglobulin, such as human. In one embodiment, a animal can be obtained by breeding a transgenic ungulate that lacks expression of at least one allele of an endogenous immunoglobulin wherein the immunoglobulin is selected from the group consisting of heavy chain, kappa light chain and lambda light chain or any combination thereof with an ungulate that expresses an xenogenous immunoglobulin. In another embodiment, a animal can be obtained by breeding a transgenic ungulate that lacks expression of one allele of heavy chain, kappa light chain and lambda light chain with an ungulate that expresses an xenogenous, such as human, immunoglobulin. In a further embodiment, an animal can be obtained by breeding a transgenic ungulate that lacks expression of one allele of heavy chain, kappa light chain and lambda light chain and expresses an xenogenous, such as human, immunoglobulin with another transgenic ungulate that lacks expression of one allele of heavy chain, kappa light chain and lambda light chain with an ungulate and expresses an xenogenous, such as human, immunoglobulin to produce a homozygous transgenic ungulate that lacks expression of both alleles of heavy chain, kappa light chain and lambda light chain and expresses an xenogenous, such as human, immunoglobulin. Methods to produce such animals are also provided.

In one embodiment, sexually mature animals produced from nuclear transfer from donor cells that carrying a double knockout in the immunoglobulin gene, can be bred and their offspring tested for the homozygous knockout. These homozygous knockout animals can then be bred to produce more animals.

In another embodiment, oocytes from a sexually mature double knockout animal can be in vitro fertilized using wild type sperm from two genetically diverse pig lines and the embryos implanted into suitable surrogates. Offspring from these matings can be tested for the presence of the knockout, for example, they can be tested by cDNA sequencing, and/or PCR. Then, at sexual maturity, animals from each of these litters can be mated. In certain methods according to this aspect of the invention, pregnancies can be terminated early so that fetal fibroblasts can be isolated and further characterized phenotypically and/or genotypically. Fibroblasts that lack expression of the immunoglobulin gene can then be used for nuclear transfer according to the methods described herein to produce multiple pregnancies and offspring carrying the desired double knockout.

Additional Genetic Modifications

In other embodiments, animals or cells lacking expression of functional immunoglobulin, produced according to the process, sequences and/or constructs described herein, can contain additional genetic modifications to eliminate the expression of xenoantigens. The additional genetic modifications can be made by further genetically modifying cells obtained from the transgenic cells and animals described herein or by breeding the animals described herein with animals that have been further genetically modified. Such animals can be modified to elimate the expression of at least one allele of the alpha-1,3-galactosyltransferase gene, the CMP-Neu5Ac hydroxylase gene (see, for example, U.S. Ser. No. 10/863,116), the iGb3 synthase gene (see, for example, U.S. Patent Application 60/517,524), and/or the Forssman synthase gene (see, for example, U.S. Patent Application 60/568,922). In additional embodiments, the animals discloses herein can also contain genetic modifications to expresss fucosyltransferase, sialyltransferase and/or any member of the family of glucosyltransferases. To achieve these additional genetic modifications, in one embodiment, cells can be modified to contain multiple genentic modifications. In other embodiments, animals can be bred together to achieve multiple genetic modifications. In one specific embodiment, animals, such as pigs, lacking expression of functional immunoglobulin, produced according to the process, sequences and/or constructs described herein, can be bred with animals, such as pigs, lacking expression of alpha-1,3-galactosyl transferase (for example, as described in WO 04/028243).

In another embodiment, the expression of additional genes responsible for xenograft rejection can be eliminated or reduced. Such genes include, but are not limited to the CMP-NEUAc Hydroxylase Gene, the isoGloboside 3 Synthase gene, and the Forssman synthase gene. In addition, genes or cDNA encoding complement related proteins, which are responsible for the suppression of complement mediated lysis can also be expressed in the animals and tissues of the present invention. Such genes include, but are not limited to CD59, DAF, MCP and CD46 (see, for example, WO 99/53042; Chen et al. Xenotransplantation, Volume 6 Issue 3 Page 194—August 1999, which describes pigs that express CD59/DAF transgenes; Costa C et al, Xenotransplantation. January 2002;9(1):45-57, which describes transgenic pigs that express human CD59 and H-transferase; Zhao L et al.; Diamond LE et al. Transplantation. January 15, 2001;71(1):132-42, which describes a human CD46 transgenic pigs.

Additional modifications can include expression of tissue factor pathway inhibitor (TFPI). heparin, antithrombin, hirudin, TFPI, tick anticoagulant peptide, or a snake venom factor, such as described in WO 98/42850 and U.S. Pat. No. 6,423,316, entitled “Anticoagulant fusion protein anchored to cell membrane”; or compounds, such as antibodies, which down-regulate the expression of a cell adhesion molecule by the cells, such as described in WO 00/31126, entitled “Suppression of xenograft rejection by down regulation of a cell adhesion molecules” and compounds in which co-stimulation by signal 2 is prevented, such as by administration to the organ recipient of a soluble form of CTLA-4 from the xenogeneic donor organism, for eample as described in WO 99/57266, entitled “Immunosuppression by blocking T cell co-stimulation signal 2 (B7/CD28 interaction)”.

Certain aspects of the invention are described in greater detail in the non-limiting Examples that follow.

### EXAMPLES

### Example 1

### Porcine Heavy Chain Targeting and Generation of Porcine Animals that Lack Expression of Heavy Chain

A portion of the porcine Ig heavy-chain locus was isolated from a 3X redundant porcine BAC library. In general, BAC libraries can be generated by fragmenting pig total genomic DNA, which can then be used to derive a BAC library representing at least three times the genome of the whole animal. BACs that contain porcine heavy chain immunoglobulin can then be selected through hybridization of probes selective for porcine heavy chain immunoglobulin as described herein.

Sequence from a clone (Seq ID 1) was used to generate a primer complementary to a portion of the J-region (the primer is represented by Seq ID No. 2). Separately, a primer was designed that was complementary to a portion of Ig heavy-chain mu constant region (the promer is represented by Seq ID No. 3). These primers were used to amplify a fragment of porcine Ig heavy-chain (represented by Seq ID No. 4) that led the functional joining region (J-region) and sufficient flanking region to design and build a targeting vector. To maintain this fragment and sublcones of this fragment in a native state, the E. coli (Stable 2, Invitrogen cat #1026-019) that harbored these fragments was maintained at 30° C. Regions of Seq. ID No. 4 were subcloned and used to assemble a targeting vector as shown in Seq. ID No. 5. This vector was transfected into porcine fetal fibroblasts that were subsequently subjected to selection with G418. Resulting colonies were screened by PCR to detect potential targeting events (Seq ID No. 6 and Seq ID No. 7, 5′ screen prmers; and Seq ID No. 8 and Seq ID No. 9, 3′ screen primers). See FIG. 1 for a schematic illustrating the targeting. Targeting was confirmed by southern blotting. Piglets were generated by nuclear transfer using the targeted fetal fibroblasts as nuclear donors.

Nuclear Transfer.

The targeted fetal fibroblasts were used as nuclear donor cells. Nuclear transfer was performed by methods that are well known in the art (see, e.g., Dai et al., Nature Biotechnology 20: 251-255, 2002; and Polejaeva et al., Nature 407:86-90, 2000).

Oocytres were collected 46-54 h after the hCG injection by reverse flush of the oviducts using pre-warmed Dulbecco's phosphate buffered saline (PBS) containing bovine serum albumin (BSA; 4 gl⁻¹) (as described in Polejaeva, I. A., et al. (Nature 407, 86-90 (2000)). Enucleation of in vitro-matured oocytes (BioMed, Madison, Wis.) was begun between 40 and 42 hours post-maturation as described in Polejaeva, I. A., et al. (Nature 407, 86-90 (2000)). Recovered oocytes were washed in PBS containing 4 gl⁻¹ BSA at 38° C., and transferred to calcium-free phosphate-buffered NCSU-23 medium at 38° C. for transport to the laboratory. For enucleation, we incubated the oocytes in calcium-free phosphate-buffered NCSU-23 medium containing 5 μg ml⁻¹ cytochalasin B (Sigma) and 7.5 μg ml⁻¹ Hoechst 33342 (Sigma) at 38° C. for 20 min. A small amount of cytoplasm from directly beneath the first polar body was then aspirated using an 18 μM glass pipette (Humagen, Charlottesville, Va.). We exposed the aspirated karyoplast to ultraviolet light to confirm the presence of a metaphase plate.

For nuclear transfer, a single fibroblast cell was placed under the zona pellucida in contact with each enucleated oocyte. Fusion and activation were induced by application of an AC pulse of 5 V for 5 s followed by two DC pulses of 1.5 kV/cm for 60 μs each using an ECM2001 Electrocell Manipulator (BTX Inc., San Diego, Calif.). Fused embryos were cultured in NCSU-23 medium for 1-4 h at 38.6° C. in a humidified atmosphere of 5% CO₂, and then transferred to the oviduct of an estrus-synchronized recipient gilt. Crossbred gilts (large white/Duroc/landrace) (280-400 lbs) were synchronized as recipients by oral administration of 18-20 mg Regu-Mate (Altrenogest, Hoechst, Warren, N.J.) mixed into their feed. Regu-Mate was fed for 14 consecutive days. Human chorionic gonadotropin (hCG, 1,000 units; Intervet America, Millsboro, Del.) was administered intra-muscularly 105 h after the last Regu-Mate treatment. Embryo transfers were done 22-26 h after the hCG injection.

Nuclear transfer produced 18 healthy piglets from four litters. These animals have one functional wild-type Ig heavy-chain locus and one disrupted Ig heavy chain locus.

| | |
|---|---|
| Seq ID 2: primer from | ggccagacttcctcggaacagctca |
| Butler subclone to | |
| amplify J to C | |
| heavychain (637Xba5′) | |
| Seq ID 3: primer for | ttccaggagaaggtgacggagct |
| C to amplify J to C | |
| heavychain (JM1L) | |
| Seq ID 6: heavychain | tctagaagacgctggagagaggccag |
| 5′ primer for 5′ | |
| screen (HCKOXba5′2) | |
| Seq ID 7: heavychain | taaagcgcatgctccagactgcctt |
| 3′ primer for 5′ | |
| screen (5′arm5′) | |
| Seq ID 8: heavychain | catcgccttctatcgccttctt |
| 5′ primer for 3′ | |
| screen (NEO4425) | |
| Seq ID 9: heavychain | Aagtacttgccgcctctcagga |
| 3′ primer for 3′ | |
| screen (650+CA) | |

Southern blot analysis of cell and pig tissue samples. Cells or tissue samples were lysed overnight at 60° C. in lysis buffer (10 mM Tris, pH 7.5, 10 mM EDTA, 10 mM NaCl, 0.5% (w/v) Sarcosyl, 1 mg/ml proteinase K) and the DNA precipitated with ethanol. The DNA was then digested with NcoI or XbaI, depending on the probe to be used, and separated on a 1% agarose gel. After electrophoresis, the DNA was transferred to a nylon membrane and probed with digoxigenin-labeled probe (SEQ ID No 41 for NcoI digest, SEQ ID No 40 for XbaI digest). Bands were detected using a chemiluminescent substrate system (Roche Molecular Biochemicals).

Probes for Heavy Chain Southern:

HC J Probe (used with Xba I digest)

(Seq ID No 40)

CTCTGCACTCACTACCGCCGGACGCGCACTGCCGTGCTGCCCATGGACCA
CGCTGGGGAGGGGTGAGCGGACAGCACGTTAGGAAGTGTGTGTGTGCGCG
TGGGTGCAAGTCGAGCCAAGGCCAAGATCCAGGGGCTGGGCCCTGTGCCC
AGAGGAGAATGGCAGGTGGAGTGTAGCTGGATTGAAAGGTGGCCTGAAGG
GTGGGGCATCCTGTTTGGAGGCTCACTCTCAGCCCCAGGGTCTCTGGTTC
CTGCCGGGGTGGGGGGCGCAAGGTGCCTACCACACCCTGCTAGCCCCTCG
TCCAGTCCCGGGCCTGCCTCTTCACCACGGAAGAGGATAAGCCAGGCTGC
AGGCTTCATGTGCGCCGTGGAGAACCCAGTTCGGCCCTTGGAGG

HC Mu Probe (used with NcoI digest)

(Seq ID No 41)

GGCTGAAGTCTGAGGCCTGGCAGATGAGCTTGGACGTGCGCTGGGGAGTA
CTGGAGAAGGACTCCCGGGTGGGGACGAAGATGTTCAAGACGGGGGGCTG
CTCCTCTACGACTGCAGGCAGGAACGGGGCGTCACTGTGCCGGCGGCACC
CGGCCCCGCCCCCGCCACAGCCACAGGGGGAGCCCAGCTCACCTGGCCCA
GAGATGGACACGGACTTGGTGCCACTGGGGTGCTGGACCTCGCACACCAG
GAAGGCCTCTGGGTCCTGGGGGATGCTCACAGAGGGTAGGAGCACCCGGG
AGGAGGCCAAGTACTTGCCGCCTCTCAGGACGG

### Example 2

### Porcine Kappa Light Chain Targeting and Generation of Porcine Lacking Expression of Kappa Light Chain

A portion of the porcine Ig kappa-chain locus was isolated from a 3× redundant porcine BAC library. In general, BAC libraries can be generated by fragmenting pig total genomic DNA, which can then be used to derive a BAC library representing at least three times the genome of the whole animal. BACs that contain porcine kappa chain immunoglobulin can then be selected through hybridization of probes selective for porcine kappa chain immunoglobulin as described herein.

A fragment of porcine Ig light-chain kappa was amplified using a primer complementary to a portion of the J-region (the primer is represented by Seq ID No. 10) and a primer complementary to a region of kappa C-region (represented by Seq ID No. 11). The resulting amplimer was cloned into a plasmid vector and maintained in Stable2 cells at 30° C. ( Seq ID No. 12). See FIG. 2 for a schematic illustration.

Separately, a fragment of porcine Ig light-chain kappa was amplified using a primer complementary to a portion of the C-region (Seq ID No. 13) and a primer complementary to a region of the kappa enhancer region (Seq ID No. 14). The resulting amplimer was fragmented by restriction enzymes and DNA fragments that were produced were cloned, maintained in Stable2 cells at 30 degrees C. and sequenced. As a result of this sequencing, two non-overlapping contigs were assembled ( Seq ID No. 15, 5′ portion of amplimer; and Seq ID No. 16, 3′ portion of amplimer). Sequence from the downstream contig (Seq ID No. 16) was used to design a set of primers (Seq ID No. 17 and Seq ID No. 18) that were used to amplify a contiguous fragment near the enhancer (Seq ID No. 19). A subclone of each Seq ID No. 12 and Seq ID No. 19 were used to build a targeting vector (Seq ID No. 20). This vector was transfected into porcine fetal fibroblasts that were subsequently subjected to selection with G418. Resulting colonies were screened by PCR to detect potential targeting events (Seq ID No. 21 and Seq ID No. 22, 5′ screen primers; and Seq ID No. 23 and Seq Id No 43, 3′ screen primers, and Seq ID No. 24 and Seq Id No 24, endogenous screen primers). Targeting was confirmed by southern blotting. Southern blot strategy design was facilitated by cloning additional kappa sequence, it corresponds to the template for germline kappa transcript (Seq ID No. 25). Fetal pigs were generated by nuclear transfer.

Nuclear Transfer.

The targeted fetal fibroblasts were used as nuclear donor cells. Nuclear transfer was performed by methods that are well known in the art (see, e.g., Dai et al., Nature Biotechnology 20: 251-255, 2002; and Polejaeva et al., Nature 407:86-90, 2000).

Oocytres were collected 46-54 h after the hCG injection by reverse flush of the oviducts using pre-warmed Dulbecco's phosphate buffered saline (PBS) containing bovine serum albumin (BSA; 4 gl⁻¹) (as described in Polejaeva, I. A., et al. (Nature 407, 86-90 (2000)). Enucleation of in vitro-matured oocytes (BioMed, Madison, Wis.) was begun between 40 and 42 hours post-maturation as described in Polejaeva, I. A., et al. (Nature 407, 86-90 (2000)). Recovered oocytes were washed in PBS containing 4 gl⁻¹ BSA at 38° C., and transferred to calcium-free phosphate-buffered NCSU-23 medium at 38° C. for transport to the laboratory. For enucleation, we incubated the oocytes in calcium-free phosphate-buffered NCSU-23 medium containing 5 μg ml⁻¹ cytochalasin B (Sigma) and 7.5 μg ml⁻¹Hoechst 33342 (Sigma) at 38° C. for 20 min. A small amount of cytoplasm from directly beneath the first polar body was then aspirated using an 18 μM glass pipette (Humagen, Charlottesville, Va.). We exposed the aspirated karyoplast to ultraviolet light to confirm the presence of a metaphase plate.

For nuclear transfer, a single fibroblast cell was placed under the zona pellucida in contact with each enucleated oocyte. Fusion and activation were induced by application of an AC pulse of 5 V for 5 s followed by two DC pulses of 1.5 kV/cm for 60 μs each using an ECM2001 Electrocell Manipulator (BTX Inc., San Diego, Calif.). Fused embryos were cultured in NCSU-23 medium for 14h at 38.6° C. in a humidified atmosphere of 5% CO₂, and then transferred to the oviduct of an estrus-synchronized recipient gilt. Crossbred gilts (large white/Duroc/landrace) (280-400 lbs) were synchronized as recipients by oral administration of 18-20 mg Regu-Mate (Altrenogest, Hoechst, Warren, N.J.) mixed into their feed. Regu-Mate was fed for 14 consecutive days. Human chorionic gonadotropin (hCG, 1,000 units; Intervet America, Millsboro, Del.) was administered intramuscularly 105 h after the last Regu-Mate treatment. Embryo transfers were done 22-26 h after the hCG injection.

Nuclear transfer using kappa targeted cells produced 33 healthy pigs from 5 litters. These pigs have one functional wild-type allele of porcine Ig light-chain kappa and one disrupted Ig light-chain kappa allele.

| | |
|---|---|
| Seq ID 10: kappa J | caaggaqaccaagctggaactc |
| to C 5′ primer | |
| (kjc5′1) | |
| Seq ID 11: kappa J | tgatcaagcacaccacagagacag |
| to C 3′ primer | |
| (kjc3′2) | |
| Seq ID 13: 5′ | gatgccaagccatccgtcttacatc |
| primer for Kappa C | |
| to E (porKCS1) | |
| Seq ID 14: 3′ | tgaccaaagcagtgtgacggttgc |
| primer for Kappa C | |
| to E (porKCA1) | |
| Seq ID 17: kappa 5′ | ggatcaaacacgcatcctcatggac |
| primer for amplifi- | |
| cation of enhancer | |
| region (K3′arm1S) | |
| Seq ID 18: kappa 3′ | ggtgattggggcatggttgagg |
| primer for amplifi- | |
| cation of enhancer | |
| region (K3′arm1A) | |
| Seq ID 21: kappa | cgaacccctgtgtatatagtt |
| screen, 5′ primer, | |
| 5′ (kappa5armS) | |
| Seq ID 22: kappa | gagatgaggaagaggagaaca |
| screen, 3′ primer, | |
| 5′ (kappaNeoA) | |
| Seq ID 23: kappa | gcattgtctgagtaggtgtcatt |
| screen, 5′ primer, | |
| 3′ (kappaNeoS) | |
| Seq ID 24: kappa | cgcttcttgcagggaacacgat |
| screen, 3′ primer, | |
| 5′ (kappa5armProbe3′) | |
| Seq ID No 43, Kappa | GTCTTTGGTTTTTGCTGAGGGTT |
| screen, 3′ primer | |
| (kappa3armA2) | |

Southern blot analysis of cell and pig tissue samples. Cells or tissue samples were lysed overnight at 60° C. in lysis buffer (10 mM Tris, pH 7.5, 10 mM EDTA, 10 mM NaCl, 0.5% (w/v) Sarcosyl, 1 mg/ml proteinase K) and the DNA precipitated with ethanol. The DNA was then digested with SacI and separated on a 1% agarose gel. After electrophoresis, the DNA was transferred to a nylon membrane and probed with digoxigenin-labeled probe (SEQ ID No 42). Bands were detected using a chemiluminescent substrate system (Roche Molecular Biochemicals).

Probe for Kappa Southern:

Kappa5ArmProbe 5′/3′

(SEQ ID No 42)

gaagtgaagccagccagttcctcctgggcaggtggccaaaattacagttg
acccctcctggtctggctgaaccttgccccatatggtgacagccatctgg
ccagggcccaggtctccctctgaagcctttgggaggagagggagagtggc
tggcccgatcacagatgcggaaggggctgactcctcaaccggggtgcaga
ctctgcagggtgggtctgggcccaacacacccaaagcacgcccaggaagg
aaaggcagcttggtatcactgcccagagctaggagaggcaccgggaaaat
gatctgtccaagacccgttcttgcttctaaactccgagggggtcagatga
agtggttttgtttcttggcctgaagcatcgtgttccctgcaagaagcgg

### Example 3

### Characterization of the Porcine Lambda Gene Locus

To disrupt or disable porcine lambda, a targeting strategy has been devised that allows for the removal or disruption of the region of the lambda locus that includes a concatamer of J to C expression cassettes. BAC clones that contain portions of the porcine genome can be generated. A portion of the porcine Ig lambda-chain locus was isolated from a 3× redundant porcine BAC library in general, BAC libraries can be generated by fragmenting pig total genomic DNA, which can then be used to derive a BAC library representing at least three times the genome of the whole animal. BACs that contain porcine lambda chain immunoglobulin can then be selected through hybridization of probes selective for porcine lambdachain immunoglobulin as described herein.

BAC clones containing a lambda J-C flanking region (see FIG. 3), can be independently fragmented and subcloned into a plasmid vector. Individual subclones have been screened by PCR for the presence of a portion of the J to C intron. We have cloned several of these cassettes by amplifying from one C region to the next C region. This amplification was accomplished by using primers that are oriented to allow divergent extension within any one C region (Seq ID 26 and Seq ID 27). To obtain successful amplification, the extended products converge with extended products originated from adjacent C regions (as opposed to the same C region). This strategy produces primarily amplimers that extend from one C to the adjacent C. However, some amplimers are the result of amplification across the adjacent C and into the next C which lies beyond the adjacent C. These multi-gene amplimers contain a portion of a C, both the J and C region of the next J-C unit, the J region of the third J-C unit, and a portion of the C region of the third J-C unit. Seq ID 28 is one such amplimer and represents sequence that must be removed or disrupted.

Other porcine lambda sequences that have been cloned include: Seq ID No. 32, which includes 5′ flanking sequence to the first lambda J/C region of the porcine lambda light chain genomic sequence; Seq ID No. 33, which includes 3′ flanking sequence to the J/C cluster region of the porcine lambda light chain genomic sequence, from approximately 200 base pairs downstream of lambda J/C; Seq ID No. 34, which includes 3′ flanking sequence to the J/C cluster region of the porcine lambda light chain genomic sequence, approximately 11.8 Kb downstream of the J/C cluster, near the enhancer; Seq ID No. 35, which includes approximately 12 Kb downstream of lambda, including the enhancer region; Seq ID No. 36, which includes approximately 17.6 Kb downstream of lambda; Seq ID No. 37, which includes approximately 19.1 Kb downstream of lambda; Seq ID No. 38, which includes approximately 21.3 Kb downstream of lambda; and Seq ID No. 39, which includes approximately 27 Kb downstream of lambda.

| | |
|---|---|
| Seq ID 26: 5′ primer | ccttcctcctgcacctgtcaac |
| for lambda C to C | |
| amplimer (lamC5′) | |
| Seq ID 27: 3′ primer | tagacacaccagggtggccttg |
| for lambda C to C | |
| amplimer (lamC3′) | |

### Example 4

### Production of Targeting Vectors for the Lambda Gene

In one example, a vector has been designed and built with one targeting arm that is homologous to a region upstream of J1 and the other arm homologous to a region that is downstream of the last C (see FIG. 4). One targeting vector is designed to target upstream of J1. This targeting vector utilizes a selectable marker that can be selected for or against. Any combination of positive and negative selectable markers described herein or known in the art can be used. A fusion gene composed of the coding region of Herpes simplex thymidine kinase (TK) and the Tn5 aminoglycoside phosphotransferase (Neo resistance) genes is used. This fusion gene is flanked by recognition sites for any site specific recombinase (SSRRS) described herein or known in the art, such as lox sites. Upon isolation of targeted cells through the use of G418 selection, Cre is supplied in trans to delete the marker gene (See FIG. 5). Cells that have deleted the marker gene are selected by addition of any drug known in the art that can be metabolized by TK into a toxic product, such as ganciclovir. The resulting genotype is then targeted with a second vector. The second targeting vector (FIG. 6) is designed to target downstream of last C and uses a positive/negative selection system that is flanked on only one side by a specific recombination site (lox). The recombination site is placed distally in relation to the first targeting event. Upon isolation of the targeted genotype, Cre is again supplied in trans to mediate deletion from recombination site provided in the first targeting event to the recombination site delivered in the second targeting event. The entire J to C cluster will be removed. The appropriate genotype is again selected by administration of ganciclovir.

In another example, insertional targeting vectors are used to disrupt each C regions independently. An insertional targeting vector will be designed and assembled to disrupt individual C region genes. There are at least 3 J to C regions in the J-C cluster. We will begin the process by designing vectors to target the first and last C regions and will include in the targeting vector site-specific recombination sites. Once both insertions have been made, the intervening region will be deleted with the site-specific recombinase.

### Example 5

### Crossbreeding of Heavy Chain Single Knockout with Kappa Single Knockout Pigs

To produce pigs that have both one disrupted Ig heavy chain locus and one disrupted Ig light-chain kappa allele, single knockout animals were crossbred. The first pregnancy yielded four fetuses, two of which screened positive by both PCR and Southern for both heavy-chain and kappa targeting events (see examples 1 and 2 for primers). Fetal fibroblasts were isolated, expanded and frozen. A second pregnancy resulting from the mating of a kappa single knockout with a heavy chain single knockout produced four healthy piglets.

Fetal fibroblast cells that contain a heavy chain single knockout and a kappa chain single knockout will be used for further targeting. Such cells will be used to target the lambda locus via the methods and compositins described herein. The resulting offspring will be hereozygous knockouts for heavy chain, kappa chain and lambda chain. These animals will be further crossed with animals containing the human Ig genes as decsibed herein and then crossbred with other single Ig knockout animals to produce porcine Ig double knockout animals with human Ig replacement genes.

This invention has been described with reference to its preferred embodiments. Variations and modifications of the invention, will be obvious to those skilled in the art from the foregoing detailed description of the invention.

We claim:

1. A transgenic ungulate that lacks any expression of functional endogenous immunoglobulins.

2. The transgenic ungulate of claim 1, wherein the ungulate lacks any expression of endogenous heavy chain immunoglobulins.

3. The transgenic ungulate of claim 1, wherein the ungulate lacks any expression of endogenous light chain immunoglobulins.

4. The transgenic ungulate of claim 3, wherein the ungulate lacks any expression of endogenous kappa chain immunoglobulin.

5. The transgenic ungulate of claim 3, wherein the ungulate lacks any expression of endogenous lambda chain immunoglobulin.

6. The transgenic ungulate of claim 1, wherein the ungulate is selected from the group consisting of a porcine, bovine, ovine and caprine.

7. The transgenic ungulate of claim 6, wherein the ungulate is a porcine.

8. The transgenic ungulate of claim 1, wherein the ungulate is produced via nuclear transfer.

9. The transgenic ungulate of claim 1, wherein the ungulate expresses an exogenous immunoglobulin loci.

10. The transgenic ungulate of claim 9, wherein the exogeous immunoglobulin loci is a heavy chain immunoglobulin or fragment thereof.

11. The transgenic ungulate of claim 9, wherein the exogeous immunoglobulin loci is a light chain immunoglobulin or fragment thereof.

12. The transgenic ungulate of claim 11, wherein the light chain locus is a kappa chain locus or fragment thereof.

13. The transgenic ungulate of claim 11, wherein the light chain locus is a lambda chain locus or fragment thereof.

14. The transgenic ungulate of claim 9, wherein the xenogenous locus is a human immunoglobulin locus or fragment thereof.

15. The transgenic ungulate of claim 9, wherein an artificial chromosome contains the xenogenous immunoglobulin.

15. The transgenic ungulate of claim 15, wherein the artificial chromosomes comprise a mammalian artificial chromosome.

16. The transgenic ungulate of claim 15, wherein the mammalian artificial chromosome comprises one or more of human chromosome 14, human chromosome 2, and human chromosome 22 or fragments thereof.

17. A transgenic mammal that lacks any expression of an endogenous lambda chain immunoglobulin.

18. A transgenic ungulate that expresses a xenogenous immunoglobulin loci or fragment thereof, wherein the immunoglobulin is expressed from an immunoglobulin locus that is integrated within an endogenous ungulate chromosome.

19. The transgenic ungulate of claim 18, wherein the xenogenous immunoglobulin is a human immunoglobulin or fragment thereof.

20. The transgenic ungulate of claim 18, wherein the xenogenous immunoglobulin locus is inherited by offspring.

21. The transgenic ungulate of claim 18, wherein the xenogenous immunoglobulin locus is inherited through the male germ line by offspring.

22. The transgenic ungulate of claim 18, wherein the ungulate is a porcine, sheep, goat or cow.

23. The transgenic ungulate of claim 22, wherein the ungulate is a porcine.

24. The transgenic ungulate of claim 18, wherein the ungulate is produced through nuclear transfer.

25. The transgenic ungulate of claim 18, wherein the immunoglobulin loci are expressed in B cells to produce xenogenous immunoglobulin in response to exposure to one or more antigens.

26. The transgenic ungulateof claim 18, wherein an artificial chromosome comprises the xenogenous immunoglobulin.

27. The transgenic ungulate of claim 18, wherein the artificial chromosome comprises a mammalian artificial chromosome.

28. The transgenic ungulate of claim 27, wherein the artificial chromosomes comprises a yeast artificial chromosome.

29. The transgenic ungulate of claim 26, wherein the artificial chromosome comprises one or more of human chromosome 14, human chromosome 2, and human chromosome 22 or fragment thereof.

30. A transgenic ungulate cell, tissue or organ derived from the transgenic ungulate of claim 1.

31. A transgenic ungulate cell, tissue or organ derived from the transgenic ungulate of claim 18.

32. The cell of claim 30 or 31, wherein the cell is a somatic, reproductive or germ cell.

33. The cell of claim 32, wherein the cell is a B cell.

34. The cell of claim 33, wherein the cell is a fibroblast cell.

35. A porcine animal comprising a xenogenous immunoglobulin locus.

36. The porcine of claim 35, wherein an artificial chromosome contains the xenogenous locus.

37. The porcine of claim 36, wherein the artificial chromosome comprises one or more xenogenous immunoglobulin loci that undergo rearrangement and can produce a xenogenous immunoglobulin in response to exposure to one or more antigens.

38. The procine cell derived from the animal of claim 35.

39. The procine cell of claim 36, wherein the cell is a somatic cell, a B cell or a fibroblast.

40. The porcine of claim 35, wherein the xenogenous immunoglobulin is a human immunoglobulin.

41. The porcine of claim 36, wherein the one or more artificial chromosomes comprise a mammalian artificial chromosome.

42. The porcine of claim 41, wherein the mammalian artificial chromosome comprises one or more of human chromosome 14, human chromosome 2, and human chromosome 22 or fragments thereof.

43. A method of producing xenogenous antibodies, the method comprising the steps of: (a) administering one or more antigens of interest to an ungulate whose cells comprise one or more artificial chromosomes and lack any expression of functional endogenous immunoglobulin, each artificial chromosome comprising one or more xenogenous immunoglobulin loci that undergo rearrangement, resulting in production of xenogenous antibodies against the one or more antigens; and (b) recovering the xenogenous antibodies from the ungulate.

44. The method of claim 43, wherein the immunoglobulin loci undergo rearrangement in a B cell.

45. The method of claim 43, wherein the exogeous immunoglobulin loci is a heavy chain immunoglobulin or fragment thereof.

46. The method of claim 43, wherein the exogeous immunoglobulin loci is a light chain immunoglobulin or fragment thereof.

47. The method of claim 43, wherein the xenogenous locus is a human immunoglobulin locus or fragment thereof.

48. The method of claim 43, wherein an artificial chromosome contains the xenogenous immunoglobulin.

49. The method of claim 48, wherein the artificial chromosomes comprise a mammalian artificial chromosome.

50. The method of claim 49, wherein the mammalian artificial chromosome comprises one or more of human chromosome 14, human chromosome 2, and human chromosome 22 or fragments thereof.

51. An isolated nucleotide sequence comprising porcine heavy chain immunoglobulin or fragment thereof, wherein the heavy chain immunoglobulin includes at least one joining region and at least one constant immunoglobulin region.

52. The nucleotide sequence of claim 51, wherein the heavy chain immunoglobulin comprises at least one variable region, at least two diversity regions, at least four joining regions and at least one constant region.

53. The nucleotide sequence of claim 52, wherein the heavy chain immunoglobulin comprises Seq ID No. 29.

54. The nucleotide sequence of claim 51, wherein the heavy chain immunoglobulin comprises Seq ID No. 4.

55. The nucleotide sequence of claim 53 or 54, wherein the sequence is at least 80, 85, 90, 95, 98 or 99% homologous to Seq ID Nos 4 or 29.

56. The nucleotide sequence of claim 53 or 54, wherein the sequence contains at least 17, 20, 25 or 30 contiguous nucleotides of Seq ID No 4 or residues 1-9,070 of Seq ID No 29.

57. The nucleotide sequence of claim 53 or 54, wherein the sequence comprises residues 9,070-11039 of Seq ID No 29.

58. An isolated nucleotide sequences that hybridizes to Seq ID No 4 or 29.

59. A targeting vector comprising:
(a) a first nucleotide sequence comprising at least 17 contiguous nucleic acids homologous to SEQ ID No 29;
(b) a selectable marker gene; and
(c) a second nucleotide sequence comprising at least 17 contiguous nucleic acids homologous to SEQ ID No 29, which does not overlap with the first nucleotide sequence.

60. The targeting vector of claim 59 wherein the selectable marker comprises an antibiotic resistence gene.

61. The targeting vector of claim 59 wherein the first nucleotide sequence represents the 5′ recombination arm.

62. The targeting vector of claim 59 wherein the second nucleotide sequence represents the 3′ recombination arm.

63. A cell transfected with the targeting vector of claim 59.

64. The cell of claim 63 wherein at least one allele of a porcine heavy chain immunoglobulin locus has been rendered inactive.

65. A porcine animal comprising the cell of claim 64.

66. An isolated nucleotide sequence comprising an ungulate kappa light chain immunoglobulin locus or fragment thereof.

67. The nucleotide sequence of claim 66, wherein the ungulate is a porcine.

68. The nucleotide sequence of claim 66, wherein the ungulate kappa light chain immunoglobulin locus comprises at least one joining region, one constant region and/or one enhancer region.

69. The nucleotide sequence of claim 66, wherein the nucleotide sequence comprises at least five joining regions, one constant region and one enhancer region.

70. The nucleotide sequence of claim 69 comprising Seq ID No. 30.

71. The nucleotide sequence of claim 69 comprising Seq ID No. 12.

72. The nucleotide sequence of claim 70 or 71, wherein the sequence contains at least 17, 20, 25 or 30 contiguous nucleotides of Seq ID No 12 or 30.

73. An isolated nucleotide sequences that hybridizes to Seq ID No 12 or 30.

74. A targeting vector comprising:
(a) a first nucleotide sequence comprising at least 17 contiguous nucleic acids homologous to SEQ ID No 30;
(b) a selectable marker gene; and
(c) a second nucleotide sequence comprising at least 17 contiguous nucleic acids homologous to SEQ ID No 30, which does not overlap with the first nucleotide sequence.

75. The targeting vector of claim 74 wherein the selectable marker comprises an antibiotic resistence gene.

76. The targeting vector of claim 74 wherein the first nucleotide sequence represents the 5′ recombination arm.

77. The targeting vector of claim 74 wherein the second nucleotide sequence represents the 3′ recombination arm.

78. A cell transfected with the targeting vector of claim 74.

79. The cell of claim 78 wherein at least one allele of a kappa chain immunoglobulin locus has been rendered inactive.

80. A porcine animal comprising the cell of claim 79.

81. An isolated nucleotide sequence comprising an ungulate lambda light chain immunoglobulin locus.

82. The nucleotide sequence of claim 81, wherein the ungulate is a porcine.

83. The nucleotide sequence of claim 81, wherein the ungulate is a bovine.

84. The nucleotide sequence of claim 81, wherein the ungulate lambda light chain immunoglobulin locus comprises a concatamer of J to C units.

85. The nucleotide sequence of claim 81, wherein the ungulate lambda light chain immunoglobulin locus comprises at least one joining region-constant region pair and/or at least one variable region, for example, as represented by Seq ID No. 31.

86. The nucleotide sequence of claim 82 comprising Seq ID No. 28.

87. The nucleotide sequence of claim 83 comprising Seq ID No. 31.

88. The nucleotide sequence of claim 86 or 87, wherein the sequence contains at least 17, 20, 25 or 30 contiguous nucleotides of Seq ID No 28 or 31.

89. An isolated nucleotide sequences that hybridizes to Seq ID No 28 or 31.

90. A targeting vector comprising:
(a) a first nucleotide sequence comprising at least 17 contiguous nucleic acids homologous to SEQ ID No 28 or 31;
(b) a selectable marker gene; and
(c) a second nucleotide sequence comprising at least 17 contiguous nucleic acids homologous to SEQ ID No 28 or 31, which does not overlap with the first nucleotide sequence.

91. The targeting vector of claim 90 wherein the selectable marker comprises an antibiotic resistence gene.

92. The targeting vector of claim 90 wherein the first nucleotide sequence represents the 5′ recombination arm.

93. The targeting vector of claim 90 wherein the second nucleotide sequence represents the 3′ recombination arm.

94. A cell transfected with the targeting vector of claim 90.

95. The cell of claim 94 wherein at least one allele of a lambda chain immunoglobulin locus has been rendered inactive.

96. A porcine animal comprising the cell of claim 95.

97. A method to circularize at least 100 kb of DNA, wherein the DNA can then be integrated into a host genome via a site specific recombinase.

98. The method of claim 97, wherein at least 100, 200, 300, 400, 500, 1000, 2000, 5000, 10,000 kb of DNA can be circularized.

99. The method of claim 97, wherein the circularization of the DNA can be accomplished by attaching site specific recombinase target sites at each end of the DNA sequence and then applying a site specific recombinase to the DNA sequence.

100. The method of claim 97, wherein the site specific recombinase target site is Lox.

101. The method of claim 97, wherein an artificial chromosome contains the DNA sequence.

102. The method of claim 101, wherein the artificial chromosome is a yeast artificial chromosome or a mammalian artificial chromosome.

103. The method of claim 101, wherein the artificial chromosome comprises a DNA sequence that encodes a human immunoglobulin locus or fragment thereof.

104. The method of claim 103, the human immunoglobulin locus or fragment thereof comprises human chromosome 14, human chromosome 2, and/or human chromosome 22.

105. A transgenic ungulate that lacks expression of at least one allele of an endogenous immunoglobulin wherein the immunoglobulin is selected from the group consisting of heavy chain, kappa light chain and lambda light chain or any combination thereof.

106. The transgenic ungulate of claim 105, wherein xenogenous immunoglobulin is expressed.

107. A method to produce the transgenic ungulate of claim 106, wherein a transgenic ungulate that lacks expression of at least one allele of an endogenous immunoglobulin wherein the immunoglobulin is selected from the group consisting of heavy chain, kappa light chain and lambda light chain or any combination thereof is bred with an ungulate that expresses an xenogenous immunoglobulin.

108. The transgenic ungulate of any of claims 105-107, wherein the ungulate is a porcine.

109. The transgenic ungulate of claim 106 or 107, wherein the xenogenous immunoglobulin is a human immunoglobulin locus or fragment thereof.

110. The transgenic ungulate of claim 109, wherein an artificial chromosome contains the human immunoglobulin locus or fragment thereof.

111. A cell derived from the ungulate of claim 105.

112. The transgenic ungulate of claim 1, 18, 105 or 106, further comprising an additional genetic modifications to eliminate the expression of a xenoantigen.

113. The transgenic ungulate of claim 112, wherein the ungulate lacks expression of at least one allele of the alpha-1,3-galactosyltransferase gene.

114. The transgenic ungulate of claim 112, wherein the ungulate is a porcine.


SEQUENCE LISTING (43 sequences)

SEQ ID NO: 1 - DNA, 666 bp, Sus scrofa
tctagaagacgctggagagaggccagacttcctcggaacagctcaaagagctctgtcaaagccagatcccatcacacgtgggcaccaataggccatgccagcctccaagggccgaactgggttctccacggcgcacatgaagcctgcagcctggcttatcctcttccgtggtgaagaggcaggcccgggactggacgaggggctagcagggtgtggtaggcaccttgcgccccccaccccggcaggaaccagagaccctggggctgagagtgagcctccaaacaggatgccccacccttcaggccacctttcaatccagctacactccacctgccattctcctctgggcacagggcccagcccctggatcttggccttggctcgacttgcacccacgcgcacacacacacttcctaacgtgctgtccgctcacccctccccagcgtggtccatgggcagcacggcagtgcgcgtccggcggtagtgagtgcagaggtcccttcccctcccccaggagccccaggggtgtgtgcagatctgggggctcctgtcccttacaccttcatgcccctcccctcatacccaccctccaggcgggaggcagcgagacctttgcccagggactcagccaacgggcacacgggaggccagccctcagcagctggg

SEQ ID NO: 2 - DNA, 25 bp, Sus scrofa
ggccagacttcctcggaacagctca

SEQ ID NO: 3 - DNA, 23 bp, Sus scrofa
ttccaggagaaggtgacggagct

SEQ ID NO: 4 - DNA, 9175 bp, Sus scrofa
ggccagacttcctcggaacagctcaaagagctctgtcaaagccagatcccatcacacgtgggcaccaataggccatgccagcctccaagggccgaactgggttctccacggcgcacatgaagcctgcagcctggcttatcctcttccgtggtgaagaggcaggcccgggactggacgaggggctagcagggtgtggtaggcaccttgcgccccccaccccggcaggaaccagagaccctggggctgagagtgagcctccaaacaggatgccccacccttcaggccacctttcaatccagctacactccacctgccattctcctctgggcacagggcccagcccctggatcttggccttggctcgacttgcacccacgcgcacacacacacttcctaacgtgctgtccgctcacccctccccagcgtggtccatgggcagcacggcagtgcgcgtccggcggtagtgagtgcagaggtcccttcccctcccccaggagccccaggggtgtgtgcagatctgggggctcctgtcccttacaccttcatgcccctcccctcatacccaccctccaggcgggaggcagcgagacctttgcccagggactcagccaacgggcacacgggaggccagccctcagcagctggctcccaaagaggaggtgggaggtaggtccacagctgccacagagagaaaccctgacggaccccacaggggccacgccagccggaaccagctccctcgtgggtgagcaatggccagggccccgccggccaccacggctggccttgcgccagctgagaactcacgtccagtgcagggagactcaagacagcctgtgcacacagcctcggatctgctcccatttcaagcagaaaaaggaaaccgtgcaggcagccctcagcatttcaaggattgtagcagcggccaactattcgtcggcagtggccgattagaatgaccgtggagaagggcggaagggtctctcgtgggctctgcggccaacaggccctggctccacctgcccgctgccagcccgaggggcttgggccgagccaggaaccacagtgctcaccgggaccacagtgactgaccaaactcccggccagagcagccccaggccagccgggctctcgccctggaggactcaccatcagatgcacaagggggcgagtgtggaagagacgtgtcgcccgggccatttgggaaggcgaagggaccttccaggtggacaggaggtgggacgcactccaggcaagggactgggtccccaaggcctggggaaggggtactggcttgggggttagcctggccagggaacggggagcggggcggggggctgagcagggaggacctgacctcgtgggagcgaggcaagtcaggcttcaggcagcagccgcacatcccagaccaggaggctgaggcaggaggggcttgcagcggggcgggggcctgcctggctccgggggctcctgggggacgctggctcttgtttccgtgtcccgcagcacagggccagctcgctgggcctatgcttaccttgatgtctggggccggggcgtcagggtcgtcgtctcctcaggggagagtcccctgaggctacgctggggggggactatggcagctccaccaggggcctggggaccaggggcctggaccaggctgcagcccggaggacgggcagggctctggctctccagcatctggccctcggaaatggcagaacccctggcgggtgagcgagctgagagcgggtcagacagacaggggccggccggaaaggagaagttgggggcagagcccgccaggggccaggcccaaggttctgtgtgccagggcctgggtgggcacattggtgtggccatggctacttagattcgtggggccagggcatcctggtcaccgtctcctcaggtgagcctggtgtctgatgtccagctaggcgctggtgggccgcgggtgggcctgtctcaggctagggcaggggctgggatgtgtatttgtcaaggaggggcaacagggtgcagactgtgcccctggaaacttgaccactggggcaggggcgtcctggtcacgtctcctcaggtaagacggccctgtgcccctctctcgcgggactggaaaaggaattttccaagattccttggtctgtgtggggccctctggggcccccgggggtggctcccctcctgcccagatggggcctcggcctgtggagcacgggctgggcacacagctcgagtctagggccacagaggcccgggctcagggctctgtgtggcccggcgactggcagggggctcgggtttttggacaccccctaatgggggccacagcactgtgaccatcttcacagctggggccgaggagtcgaggtcaccgtctcctcaggtgagtcctcgtcagccctctctcactctctggggggttttgctgcattttgtgggggaaagaggatgcctgggtctcaggtctaaaggtctagggccagcgccggggcccaggaaggggccgaggggccaggctcggctcggccaggagcagagcttccagacatctcgcctcctggcggctgcagtcaggcctttggccgggggggtctcagcaccaccaggcctcttggctcccgaggtccccggccccggctgcctcaccaggcaccgtgcgcggtgggcccgggctcttggtcggccaccctttcttaactgggatccgggcttagttgtcgcaatgtgacaacgggctcgaaagctggggccaggggaccctagttacgacgcctcgggtgggtgtcccgcacccctccccactttcacggcactcggcgagacctggggagtcaggtgttggggacactttggaggtcaggaacgggagctggggagagggctctgtcagcggggtccagagatgggccgccctccaaggacgccctgcgcggggacaagggcttcttggcctggcctggccgcttcacttgggcgtcagggggggcttcccggggcaggcggtcagtcgaggcgggttggaattctgagtctgggttcggggttcggggttcggccttcatgaacagacagcccaggcgggccgttgtttggcccctgggggcctggttggaatgcgaggtctcgggaagtcaggagggagcctggccagcagagggttcccagccctgcggccgagggacctggagacgggcagggcattggccgtcgcagggccaggccacaccccccaggtttttgtggggcgagcctggagattgcaccactgtgattactatgctatggatctctggggcccaggcgttgaagtcgtcgtgtcctcaggtaagaacggccctccagggcctttaatttctgctctcgtctgtgggcttttctgactctgatcctcgggaggcgtctgtgccccccccggggatgaggccggcttgccaggaggggtcagggaccaggagcctgtgggaagttctgacgggggctgcaggcgggaagggccccaccggggggcgagccccaggccgctgggcggcaggagacccgtgagagtgcgccttgaggagggtgtctgcggaaccacgaacgcccgccgggaagggcttgctgcaatgcggtcttcagacgggaggcgtcttctgccctcaccgtctttcaagcccttgtgggtctgaaagagccatgtcggagagagaagggacaggcctgtcccgacctggccgagagcgggcagccccgggggagagcggggcgatcggcctgggctctgtgaggccaggtccaagggaggacgtgtggtcctcgtgacaggtgcacttgcgaaaccttagaagacggggtatgttggaagcggctcctgatgtttaagaaaagggagactgtaaagtgagcagagtcctcaagtgtgttaaggttttaaaggtcaaagtgttttaaacctttgtgactgcagttagcaagcgtgcggggagtgaatggggtgccagggtggccgagaggcagtacgagggccgtgccgtcctctaattcagggcttagttttgcagaataaagtcggcctgttttctaaaagcattggtggtgctgagctggtggaggaggccgcgggcagccctggccacctgcagcaggtggcaggaagcaggtcggccaagaggctattttaggaagccagaaaacacggtcgatgaatttatagcttctggtttccaggaggtggttgggcatggctttgcgcagcgccacagaaccgaaagtgcccactgagaaaaaacaactcctgcttaatttgcatttttctaaaagaagaaacagaggctgacggaaactggaaagttcctgttttaactactcgaattgagttttcggtcttagcttatcaactgctcacttagattcattttcaaagtaaacgtttaagagccgaggcattcctatcctcttctaaggcgttattcctggaggctcattcaccgccagcacctccgctgcctgcaggcattgctgtcaccgtcaccgtgacggcgcgcacgattttcagttggcccgcttcccctcgtgattaggacagacgcgggcactctggcccagccgtcttggctcagtatctgcaggcgtccgtctcgggacggagctcaggggaagagcgtgactccagttgaacgtgatagtcggtgcgttgagaggagacccagtcgggtgtcgagtcagaaggggcccggggcccgaggccctgggcaggacggcccgtgccctgcatcacgggcccagcgtcctagaggcaggactctggtggagagtgtgagggtgcctggggcccctccggagctggggccgtgcggtgcaggttgggctctcggcgcggtgttggctgtttctgcgggatttggaggaattcttccagtgatgggagtcgccagtgaccgggcaccaggctggtaagagggaggccgccgtcgtggccagagcagctgggagggttcggtaaaaggctcgcccgtttcctttaatgaggacttttcctggagggcatttagtctagtcgggaccgttttcgactcgggaagagggatgcggaggagggcatgtgcccaggagccgaaggcgccgcggggagaagcccagggctctcctgtccccacagaggcgacgccactgccgcagacagacagggcctttccctctgatgacggcaaaggcgcctcggctcttgcggggtgctgggggggagtcgccccgaagccgctcacccagaggcctgaggggtgagactgaccgatgcctcttggccgggcctggggccggaccgagggggactccgtggaggcagggcgatggtggctgcgggagggaaccgaccctgggccgagcccggcttggcgattcccgggcgagggccctcagccgaggcgagtgggtccggcggaaccaccctttctggccagcgccacagggctctcgggactgtccggggcgacgctgggctgcccgtggcaggcctgggctgacctggacttcaccagacagaacagggctttcagggctgagctgagccaggtttagcgaggccaagtggggctgaaccaggctcaactggcctgagctgggttgagctgggctgacctgggctgagctgagctgggctgggctgggctgggctgggctgggctgggctggactggctgagctgagctgggttgagctgagctgagctggcctgggttgagctgggctgggttgagctgagctgggttgagctgggttgagctgggttgatctgagctgagctgggctgagctgagctaggctggggtgagctgggctgagctggtttgagttgggttgagctgagctgagctgggctgtgctggctgagctaggctgagctaggctaggttgagctgggctgggctgagctgagctaggctgggctgatttgggctgagctgagctgagctaggctgcgttgagctggctgggctggattgagctggctgagctggctgagctgggctgagctggcctgggttgagctgagctggactggtttgagctgggtcgatctgggttgagctgtcctgggttgagctgggctgggttgagctgagctgggttgagctgggctcagcagagctgggttgggctgagctgggttgagctgagctgggctgagctggcctgggttgagctgggctgagctgagctgggctgagctggcctgtgttgagctgggctgggttgagctgggctgagctggattgagctgggttgagctgagctgggctgggctgtgctgactgagctgggctgagctaggctggggtgagctgggctgagctgatccgagctaggctgggctggtttgggctgagctgagctgagctaggctggattgatctggctgagctgggttgagctgagctgggctgagctggtctgagctggcctgggtcgagctgagctggactggtttgagctgggtcgatctgggctgagctggcctgggttgagctgggctgggttgagctgagctgggttgagctgggctgagctgagggctggggtgagctgggctgaactagcctagctaggttgggctgagctgggctggtttgggctgagctgagctgagctaggctgcattgagcaggctgagctgggctgagcaggcctggggtgagctgggctaggtggagctgagctgggtcgagctgagttgggctgagctggcctgggttgaggtaggctgagctgagctgagctaggctgggttgagctggctgggctggtttgcgctgggtcaagctgggccgagctggcctgggttgagctgggctcggttgagctgggctgagctgagccgacctaggctgggatgagctgggctgatttgggctgagctgagctgagctaggctgcattgagcaggctgagctgggcctggagcctggcctggggtgagctgggctgagctgcgctgagctaggctgggttgagctggctgggctggtttgcgctgggtcaagctgggccgagctggcctgggatgagctgggccggtttgggctgagctgagctgagctaggctgcattgagcaggctgagctgggctgagctggcctggggtgagctgggctgagctaagctgagctgggctggtttgggctgagctggctgagctgggtcctgctgagctgggctgagctgaccaggggtgagctgggctgagttaggctgggctcagctaggctgggttgatctggcagggctggtttgcgctgggtcaagctcccgggagatggcctgggatgagctgggctggtttgggctgagctgagctgagctgagctaggctgcattgagcaggctgagctgggctgagctggcctggggtgagctgggctgggtggagctgagctgggctgaactgggctaagctggctgagctggatcgagctgagctgggctgagctggcctggggttagctgggctgagctgagctgagctaggctgggttgagctggctgggctggtttgcgctgggtcaagctgggccgagctggcctgggttgagctgggctgggctgagctgagctaggctgggttgagctgggctgggctgagctgagctaggctgcattgagctggctgggatggattgagctggctgagctggctgagctggctgagctgggctgagctggcctgggttgagctgggctgggttgagctgagctgggctgagctgggctcagcagagctgggttgagctgagctgggttgagctggggtgagctgggctgagcagagctgggttgagctgagctgggttgagctgggctcgagcagagctgggttgagctgagctgggttgagctgggctcagcagagctgggttgagctgagctgggttgagctgggctgagctagctgggctcagctaggctgggttgagctgagctgggctgaactgggctgagctgggctgaactgggctgagctgggctgagctgggctgagcagagctgggctgagcagagctgggttggtctgagctgggttgagctgggctgagctgggctgagcagagttgggttgagctgagctgggttcagctgggctgagctaggctgggttgagctgggttgagttgggctgagctgggctgggttgagcggagctgggctgaactgggctgagctgggctgagcggaactgggttgatctgaattgagctgggctgagccgggctgagccgggctgagctgggctaggttgagcttgggtgagcttgcctcagctggtctgagctaggttgggtggagctaggctggattgagctgggctgagctgagctgatctggcctcagctgggctgaggtaggctgaactgggctgtgctgggctgagctgagctgagccagtttgagctgggttgagctgggctgagctgggctgtgttgatctttcctgaactgggctgagctgggctgagctggcctagctggattgaacgggggtaagctgggccaggctggactgggctgagctgagctaggctgagctgagttgaattgggttaagctgggctgagatgggctgagctgggctgagctgggttgagccaggtcggactgggttaccctgggccacactgggctgagctgggcggagctcgattaacctggtcaggctgagtcgggtccagcagacatgcgctggccaggctggcttgacctggacacgttcgatgagctgccttgggatggttcacctcagctgagccaggtggctccagctgggctgagctggtgaccctgggtgacctcggtgaccaggttgtcctgagtccgggccaagccgaggctgcatcagactcgccagacccaaggcctgggccccggctggcaagccaggggcggtgaaggctgggctggcaggactgtcccggaaggaggtgcacgtggagccgcccggaccccgaccggcaggacctggaaagacgcctctcactcccctttctcttctgtcccctctcgggtcctcagagagccagtctgccccgaatctctaccccctcgtctcctgcgtcagccccccgtccgatgagagcctggtggccctgggctgcctggcccgggacttcctgcccagctccgtcaccttctcctggaa

SEQ ID NO: 5 - DNA, 9200 bp, Sus scrofa
ggccagacttcctcggaacagctcaaagagctctgtcaaagccagatcccatcacacgtgggcaccaataggccatgccagcctccaagggccgaactgggttctccacggcgcacatgaagcctgcagcctggcttatcctcttccgtggtgaagaggcaggcccgggactggacgaggggctagcagggtgtggtaggcaccttgcgccccccaccccggcaggaaccagagaccctggggctgagagtgagcctccaaacaggatgccccacccttcaggccacctttcaatccagctacactccacctgccattctcctctgggcacagggcccagcccctggatcttggccttggctcgacttgcacccacgcgcacacacacacttcctaacgtgctgtccgctcacccctccccagcgtggtccatgggcagcacggcagtgcgcgtccggcggtagtgagtgcagaggtcccttcccctcccccaggagccccaggggtgtgtgcagatctgggggctcctgtcccttacaccttcatgcccctcccctcatacccaccctccaggcgggaggcagcgagacctttgcccagggactcagccaacgggcacacgggaggccagccctcagcagctggctcccaaagaggaggtgggaggtaggtccacagctgccacagagagaaaccctgacggaccccacaggggccacgccagccggaaccagctccctcgtgggtgagcaatggccagggccccgccggccaccacggctggccttgcgccagctgagaactcacgtccagtgcagggagactcaagacagcctgtgcacacagcctcggatctgctcccatttcaagcagaaaaaggaaaccgtgcaggcagccctcagcatttcaaggattgtagcagcggccaactattcgtcggcagtggccgattagaatgaccgtggagaagggcggaagggtctctcgtgggctctgcggccaacaggccctggctccacctgcccgctgccagcccgaggggcttgggccgagccaggaaccacagtgctcaccgggaccacagtgactgaccaaactcccggccagagcagccccaggccagccgggctctcgccctggaggactcaccatcagatgcacaagggggcgagtgtggaagagacgtgtcgcccgggccatttgggaaggcgaagggaccttccaggtggacaggaggtgggacgcactccaggcaagggactgggtccccaaggcctggggaaggggtactggcttgggggttagcctggccagggaacggggagcggggcggggggctgagcagggaggacctgacctcgtgggagcgaggcaagtcaggcttcaggcagcagccgcacatcccagaccaggaggctgaggcaggaggggcttgcagcggggcgggggcctgcctggctccgggggctcctgggggacgctggctcttgtttccgtgtcccgcagcacagggccagctcgctgggcctatgcttaccttgatgtctggggccggggcgtcagggtcgtcgtctcctcaggggagagtcccctgaggctacgctggggggggactatggcagctccaccaggggcctggggaccaggggcctggaccaggctgcagcccggaggacgggcagggctctggctctccagcatctggccctcggaaatggcagaacccctggcgggtgagcgagctgagagcgggtcagacagacaggggccggccggaaaggagaagttgggggcagagcccgccaggggccaggcccaaggttctgtgtgccagggcctgggtgggcacattggtgtggccatggctacttagacgcgtgatcaagggcgaattccagcacactggcggccgttactagtggatcccggcgcgccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaagcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcaatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggatcgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagatgcatgctcgagcggccgccagtgtgatggatatctgcagaattcgcccttccaggcgttgaagtcgtcgtgtcctcaggtaagaacggccctccagggcctttaatttctgctctcgtctgtgggcttttctgactctgatcctcgggaggcgtctgtgccccccccggggatgaggccggcttgccaggaggggtcagggaccaggagcctgtgggaagttctgacgggggctgcaggcgggaagggccccaccggggggcgagccccaggccgctgggcggcaggagacccgtgagagtgcgccttgaggagggtgtctgcggaaccacgaacgcccgccgggaagggcttgctgcaatgcggtcttcagacgggaggcgtcttctgccctcaccgtctttcaagcccttgtgggtctgaaagagccatgtcggagagagaagggacaggcctgtcccgacctggccgagagcgggcagccccgggggagagcggggcgatcggcctgggctctgtgaggccaggtccaagggaggacgtgtggtcctcgtgacaggtgcacttgcgaaaccttagaagacggggtatgttggaagcggctcctgatgtttaagaaaagggagactgtaaagtgagcagagtcctcaagtgtgttaaggttttaaaggtcaaagtgttttaaacctttgtgactgcagttagcaagcgtgcggggagtgaatggggtgccagggtggccgagaggcagtacgagggccgtgccgtcctctaattcagggcttagttttgcagaataaagtcggcctgttttctaaaagcattggtggtgctgagctggtggaggaggccgcgggcagccctggccacctgcagcaggtggcaggaagcaggtcggccaagaggctattttaggaagccagaaaacacggtcgatgaatttatagcttctggtttccaggaggtggttgggcatggctttgcgcagcgccacagaaccgaaagtgcccactgagaaaaaacaactcctgcttaatttgcatttttctaaaagaagaaacagaggctgacggaaactggaaagttcctgttttaactactcgaattgagttttcggtcttagcttatcaactgctcacttagattcattttcaaagtaaacgtttaagagccgaggcattcctatcctcttctaaggcgttattcctggaggctcattcaccgccagcacctccgctgcctgcaggcattgctgtcaccgtcaccgtgacggcgcgcacgattttcagttggcccgcttcccctcgtgattaggacagacgcgggcactctggcccagccgtcttggctcagtatctgcaggcgtccgtctcgggacggagctcaggggaagagcgtgactccagttgaacgtgatagtcggtgcgttgagaggagacccagtcgggtgtcgagtcagaaggggcccggggcccgaggccctgggcaggacggcccgtgccctgcatcacgggcccagcgtcctagaggcaggactctggtggagagtgtgagggtgcctggggcccctccggagctggggccgtgcggtgcaggttgggctctcggcgcggtgttggctgtttctgcgggatttggaggaattcttccagtgatgggagtcgccagtgaccgggcaccaggctggtaagagggaggccgccgtcgtggccagagcagctgggagggttcggtaaaaggctcgcccgtttcctttaatgaggacttttcctggagggcatttagtctagtcgggaccgttttcgactcgggaagagggatgcggaggagggcatgtgcccaggagccgaaggcgccgcggggagaagcccagggctctcctgtccccacagaggcgacgccactgccgcagacagacagggcctttccctctgatgacggcaaaggcgcctcggctcttgcggggtgctgggggggagtcgccccgaagccgctcacccagaggcctgaggggtgagactgaccgatgcctcttggccgggcctggggccggaccgagggggactccgtggaggcagggcgatggtggctgcgggagggaaccgaccctgggccgagcccggcttggcgattcccgggcgagggccctcagccgaggcgagtgggtccggcggaaccaccctttctggccagcgccacagggctctcgggactgtccggggcgacgctgggctgcccgtggcaggcctgggctgacctggacttcaccagacagaacagggctttcagggctgagctgagccaggtttagcgaggccaagtggggctgaaccaggctcaactggcctgagctgggttgagctgggctgacctgggctgagctgagctgggctgggctgggctgggctgggctgggctgggctggactggctgagctgagctgggttgagctgagctgagctggcctgggttgagctgggctgggttgagctgagctgggttgagctgggttgagctgggttgatctgagctgagctgggctgagctgagctaggctggggtgagctgggctgagctggtttgagttgggttgagctgagctgagctgggctgtgctggctgagctaggctgagctaggctaggttgagctgggctgggctgagctgagctaggctgggctgatttgggctgagctgagctgagctaggctgcgttgagctggctgggctggattgagctggctgagctggctgagctgggctgagctggcctgggttgagctgagctggactggtttgagctgggtcgatctgggttgagctgtcctgggttgagctgggctgggttgagctgagctgggttgagctgggctcagcagagctgggttgggctgagctgggttgagctgagctgggctgagctggcctgggttgagctgggctgagctgagctgggctgagctggcctgtgttgagctgggctgggttgagctgggctgagctggattgagctgggttgagctgagctgggctgggctgtgctgactgagctgggctgagctaggctggggtgagctgggctgagctgatccgagctaggctgggctggtttgggctgagctgagctgagctaggctggattgatctggctgagctgggttgagctgagctgggctgagctggtctgagctggcctgggtcgagctgagctggactggtttgagctgggtcgatctgggctgagctggcctgggttgagctgggctgggttgagctgagctgggttgagctgggctgagctgagggctggggtgagctgggctgaactagcctagctaggttgggctgagctgggctggtttgggctgagctgagctgagctaggctgcattgagcaggctgagctgggctgagcaggcctggggtgagctgggctaggtggagctgagctgggtcgagctgagttgggctgagctggcctgggttgaggtaggctgagctgagctgagctaggctgggttgagctggctgggctggtttgcgctgggtcaagctgggccgagctggcctgggttgagctgggctcggttgagctgggctgagctgagccgacctaggctgggatgagctgggctgatttgggctgagctgagctgagctaggctgcattgagcaggctgagctgggcctggagcctggcctggggtgagctgggctgagctgcgctgagctaggctgggttgagctggctgggctggtttgcgctgggtcaagctgggccgagctggcctgggatgagctgggccggtttgggctgagctgagctgagctaggctgcattgagcaggctgagctgggctgagctggcctggggtgagctgggctgagctaagctgagctgggctggtttgggctgagctggctgagctgggtcctgctgagctgggctgagctgaccaggggtgagctgggctgagttaggctgggctcagctaggctgggttgatctggcagggctggtttgcgctgggtcaagctcccgggagatggcctgggatgagctgggctggtttgggctgagctgagctgagctgagctaggctgcattgagcaggctgagctgggctgagctggcctggggtgagctgggctgggtggagctgagctgggctgaactgggctaagctggctgagctggatcgagctgagctgggctgagctggcctggggttagctgggctgagctgagctgagctaggctgggttgagctggctgggctggtttgcgctgggtcaagctgggccgagctggcctgggttgagctgggctgggctgagctgagctaggctgggttgagctgggctgggctgagctgagctaggctgcattgagctggctgggatggattgagctggctgagctggctgagctggctgagctgggctgagctggcctgggttgagctgggctgggttgagctgagctgggctgagctgggctcagcagagctgggttgagctgagctgggttgagctggggtgagctgggctgagcagagctgggttgagctgagctgggttgagctgggctcgagcagagctgggttgagctgagctgggttgagctgggctcagcagagctgggttgagctgagctgggttgagctgggctgagctagctgggctcagctaggctgggttgagctgagctgggctgaactgggctgagctgggctgaactgggctgagctgggctgagctgggctgagcagagctgggctgagcagagctgggttggtctgagctgggttgagctgggctgagctgggctgagcagagttgggttgagctgagctgggttcagctgggctgagctaggctgggttgagctgggttgagttgggctgagctgggctgggttgagcggagctgggctgaactgggctgagctgggctgagcggaactgggttgatctgaattgagctgggctgagccgggctgagccgggctgagctgggctaggttgagcttgggtgagcttgcctcagctggtctgagctaggttgggtggagctaggctggattgagctgggctgagctgagctgatctggcctcagctgggctgaggtaggctgaactgggctgtgctgggctgagctgagctgagccagtttgagctgggttgagctgggctgagctgggctgtgttgatctttcctgaactgggctgagctgggctgagctggcctagctggattgaacgggggtaagctgggccaggctggactgggctgagctgagctaggctgagctgagttgaattgggttaagctgggctgagatgggctgagctgggctgagctgggttgagccaggtcggactgggttaccctgggccacactgggctgagctgggcggagctcgattaacctggtcaggctgagtcgggtccagcagacatgcgctggccaggctggcttgacctggacacgttcgatgagctgccttgggatggttcacctcagctgagccaggtggctccagctgggctgagctggtgaccctgggtgacctcggtgaccaggttgtcctgagtccgggccaagccgaggctgcatcagactcgccagacccaaggcctgggccccggctggcaagccaggggcggtgaaggctgggctggcaggactgtcccggaaggaggtgcacgtggagccgcccggaccccgaccggcaggacctggaaagacgcctctcactcccctttctcttctgtcccctctcgggtcctcagagagccagtctgccccgaatctctaccccctcgtctcctgcgtcagccccccgtccgatgagagcctggtggccctgggctgcctggcccgggacttcctgcccagctccgtcaccttctcctggaa

SEQ ID NO: 6 - DNA, 26 bp, Sus scrofa
tctagaagacgctggagagaggccag

SEQ ID NO: 7 - DNA, 25 bp, Sus scrofa
taaagcgcatgctccagactgcctt

SEQ ID NO: 8 - DNA, 22 bp, Sus scrofa
catcgccttctatcgccttctt

SEQ ID NO: 9 - DNA, 22 bp, Sus scrofa
aagtacttgccgcctctcagga

SEQ ID NO: 10 - DNA, 22 bp, Sus scrofa
caaggagaccaagctggaactc

SEQ ID NO: 11 - DNA, 24 bp, Sus scrofa
tgatcaagcacaccacagagacag

SEQ ID NO: 12 - DNA, 4277 bp, Sus scrofa
caaggaaccaagctggaactcaaacgtaagtcaatccaaacgttccttccttggctgtctgtgtcttacggtctctgtggctctgaaatgattcatgtgctgactctctgaaaccagactgacattctccagggcaaaactaaagcctgtcatcaaactggaaaactgagggcacattttctgggcagaactaagagtcaggcactgggtgaggaaaaacttgttagaatgatagtttcagaaacttactgggaagcaaagcccatgttctgaacagagctctgctcaagggtcaggaggggaaccagtttttgtacaggagggaagttgagacgaacccctgtgtatatggtttcggcgcggggaccaagctggagctcaaacgtaagtggctttttccgactgattctttgctgtttctaattgttggttggctttttgtccatttttcagtgttttcatcgaattagttgtcagggaccaaacaaattgccttcccagattaggtaccagggaggggacattgctgcatgggagaccagagggtggctaatttttaacgtttccaagccaaaataactggggaagggggcttgctgtcctgtgagggtaggtttttatagaagtggaagttaaggggaaatcgctatggttcacttttggctcggggaccaaagtggagcccaaaattgagtacattttccatcaattatttgtgagatttttgtcctgttgtgtcatttgtgcaagtttttgacattttggttgaatgagccattcccagggacccaaaaggatgagaccgaaaagtagaaaagagccaacttttaagctgagcagacagaccgaattgttgagtttgtgaggagagtagggtttgtagggagaaaggggaacagatcgctggctttttctctgaattagcctttctcatgggactggcttcagagggggtttttgatgagggaagtgttctagagccttaactgtgggttgtgttcggtagcgggaccaagctggaaatcaaacgtaagtgcacttttctactcctttttctttcttatacgggtgtgaaattggggacttttcatgtttggagtatgagttgaggtcagttctgaagagagtgggactcatccaaaaatctgaggagtaagggtcagaacagagttgtctcatggaagaacaaagacctagttagttgatgaggcagctaaatgagtcagttgacttgggatccaaatggccagacttcgtctgtaaccaacaatctaatgagatgtagcagcaaaaagagatttccattgaggggaaagtaaaattgttaatattgtggatcacctttggtgaagggacatccgtggagattgaacgtaagtattttttctctactaccttctgaaatttgtctaaatgccagtgttgacttttagaggcttaagtgtcagttttgtgaaaaatgggtaaacaagagcatttcatatttattatcagtttcaaaagttaaactcagctccaaaaatgaatttgtagacaaaaagattaatttaagccaaattgaatgattcaaaggaaaaaaaaattagtgtagatgaaaaaggaattcttacagctccaaagagcaaaagcgaattaattttctttgaactttgccaaatcttgtaaatgatttttgttctttacaatttaaaaaggttagagaaatgtatttcttagtctgttttctctcttctgtctgataaattattatatgagataaaaatgaaaattaataggatgtgctaaaaaatcagtaagaagttagaaaaatatatgtttatgttaaagttgccacttaattgagaatcagaagcaatgttatttttaaagtctaaaatgagagataaactgtcaatacttaaattctgcagagattctatatcttgacagatatctcctttttcaaaaatccaatttctatggtagactaaatttgaaatgatcttcctcataatggagggaaaagatggactgaccccaaaagctcagatttaagaaaacctgtttaaggaaagaaaataaaagaactgcattttttaaaggcccatgaatttgtagaaaaataggaaatattttaataagtgtattcttttattttcctgttattacttgatggtgtttttataccgccaaggaggccgtggcaccgtcagtgtgatctgtagaccccatggcggccttttttcgcgattgaatgaccttggcggtgggtccccagggctctggtggcagcgcaccagccgctaaaagccgctaaaaactgccgctaaaggccacagcaaccccgcgaccgcccgttcaactgtgctgacacagtgatacagataatgtcgctaacagaggagaatagaaatatgacgggcacacgctaatgtggggaaaagagggagaagcctgatttttattttttagagattctagagataaaattcccagtattatatccttttaataaaaaatttctattaggagattataaagaatttaaagctatttttttaagtggggtgtaattctttcagtagtctcttgtcaaatggatttaagtaatagaggcttaatccaaatgagagaaatagacgcataaccctttcaaggcaaaagctacaagagcaaaaattgaacacagcagccagccatctagccactcagattttgatcagttttactgagtttgaagtaaatatcatgaaggtataattgctgataaaaaaataagatacaggtgtgacacatctttaagtttcagaaatttaatggcttcagtaggattatatttcacgtatacaaagtatctaagcagataaaaatgccattaatggaaacttaatagaaatatatttttaaattccttcattctgtgacagaaattttctaatctgggtcttttaatcacctaccctttgaaagagtttagtaatttgctatttgccatcgctgtttactccagctaatttcaaaagtgatacttgagaaagattatttttggtttgcaaccacctggcaggactattttagggccattttaaaactcttttcaaactaagtattttaaactgttctaaaccatttagggccttttaaaaatcttttcatgaatttcaaacttcgttaaaagttattaaggtgtctggcaagaacttccttatcaaatatgctaatagtttaatctgttaatgcaggatataaaattaaagtgatcaaggcttgacccaaacaggagtatcttcatagcatatttcccctcctttttttctagaattcatatgattttgctgccaaggctattttatataatctctggaaaaaaaatagtaatgaaggttaaaagagaagaaaatatcagaacattaagaattcggtattttactaactgcttggttaacatgaaggtttttattttattaaggtttctatctttataaaaatctgttcccttttctgctgatttctccaagcaaaagattcttgatttgttttttaactcttactctcccacccaagggcctgaatgcccacaaaggggacttccaggaggccatctggcagctgctcaccgtcagaagtgaagccagccagttcctcctgggcaggtggccaaaattacagttgacccctcctggtctggctgaaccttgccccatatggtgacagccatctggccagggcccaggtctccctctgaagcctttgggaggagagggagagtggctggcccgatcacagatgcggaaggggctgactcctcaaccggggtgcagactctgcagggtgggtctgggcccaacacacccaaagcacgcccaggaaggaaaggcagcttggtatcactgcccagagctaggagaggcaccgggaaaatgatctgtccaagacccgttcttgcttctaaactccgagggggtcagatgaagtggttttgtttcttggcctgaagcatcgtgttccctgcaagaagcggggaacacagaggaaggagagaaaagatgaactgaacaaagcatgcaaggcaaaaaaggccttaggatggctgcaggaagttagttcttctgcattggctccttactggctcgtcgatcgcccacaaacaacgcacccagtggagaacttccctgttacttaaacaccattctctgtgcttgcttcctcaggggctgatgccaagccatccgtcttcatcttcccgccatcgaaggagcagttagcgaccccaactgtctctgtggtgtgcttgatca

SEQ ID NO: 13 - DNA, 24 bp, Sus scrofa
gatgccaagccatccgtcttcatc

SEQ ID NO: 14 - DNA, 24 bp, Sus scrofa
tgaccaaagcagtgtgacggttgc

SEQ ID NO: 15 - DNA, 2027 bp, Sus scrofa
gatgccaagccatccgtcttcatcttcccgccatcgaaggagcagttagcgaccccaactgtctctgtggtgtgcttgatcaataacttcttccccagagaaatcagtgtcaagtggaaagtggatggggtggtccaaagcagtggtcatccggatagtgtcacagagcaggacagcaaggacagcacctacagcctcagcagcaccctctcgctgcccacgtcacagtacctaagtcataatttatattcctgtgaggtcacccacaagaccctggcctcccctctggtcacaagcttcaacaggaacgagtgtgaggcttagaggcccacaggcccctggcctgcccccagccccagcccccctccccacctcaagcctcaggcccttgccccagaggatccttggcaatcccccagcccctcttccctcctcatcccctccccctctttggctttaaccgtgttaatactggggggtgggggaatgaataaataaagtgaacctttgcacctgtgatttctctctcctgtctgattttaaggttgttaaatgttgttttccccattatagttaatcttttaaggaactacatactgagttgctaaaaactacaccatcacttataaaattcacgccttctcagttctcccctcccctcctgtcctccgtaagacaggcctccgtgaaacccataagcacttctctttacaccctctcctgggccggggtaggagactttttgatgtcccctcttcagcaagcctcagaaccattttgagggggacagttcttacagtcacattcctgtgatctaatgactttagttaccgaaaagccagtctctcaaaaagaagggaacggctagaaaccaagtcatagaaatatatatgtataaaatatatatatatccatatatgtaaaataacaaaataatgataacagcataggtcaacaggcaacagggaatgttgaagtccattctggcacttcaatttaagggaataggatgccttcattacattttaaatacaatacacatggagagcttcctatctgccaaagaccatcctgaatgccttccacactcactacaaggttaaaagcattcattacaatgttgatcgaggagttcccgttgtggctcagcaggttaagaacgtgactggtatccaggaggatgcgggtttggtccccagcctcgctcagtggattaaggatccagtgttgctgcaagatcacgggctcagatcccgtgttctatggctatggtgtaggctggtagctgcatgcagccctaatttgacccctagcctgggaactgccatatgccacatgtgaggcccttaaaacctaaaagaaaaaaaaagaaaagaaatatcttacacccaatttatagataagagagaagctaaggtggcaggcccaggatcaaagccctacctgcctatcttgacacctgatacaaattctgtcttctagggtttccaacactgcatagaacagagggtcaaacatgctaccctcccagggactcctcccttcaaatgacataaattttgttgcccatctctgggggcaaaactcaacaatcaatggcatctctagtaccaagcaaggctcttctcatgaagcaaaactctgaagccagatccatcatgacccaaggaagtaaagacaggtgttactggttgaactgtatccttcaattcaatatgctcaatttccaactcccagtccccgtaaatacaaccccctttgggaagagagtccttgcagatgtagccacgttaaaaagagattatacagaaaggctagtgaggatgcagtgaaacgggatctttcatacattgctggtggaaatgtaaaatgctgcaggcactctagaaaataatttgccagttttttgaaaagctaaacaaaatagtttagttgcattctgggttatttatcccccagaaattaaaaattatgtccgcacaaaaacgtgtacataatcattcataacagccttgtacgaaaagctt

SEQ ID NO: 16 - DNA, 3918 bp, Sus scrofa
ggatccttaacccactaatcgaggatcaaacacgcatcctcatggacaatatgttgggttcttagcctgctgagacacaacaggaactcccctggcaccactttagaggccagagaaacagcacagataaaattccctgccctcatgaagcttatagtctagctggggagatatcataggcaagataaacacatacaaatacatcatcttaggtaataatatatactaaggagaaaattacaggggagaaagaggacaggaattgctagggtaggattataagttcagatagttcatcaggaacactgttgctgagaagataacatttaggtaaagaccgaagtagtaaggaaatggaccgtgtgcctaagtgggtaagaccattctaggcagcaggaacagcgatgaaagcactgaggtgggtgttcactgcacagagttgttcactgcacagagttgtgtggggaggggtaggtcttgcaggctcttatggtcacaggaagaattgttttactcccaccgagatgaaggttggtggattttgagcagaagaataattctgcctggtttatatataacaggatttccctgggtgctctgatgagaataatctgtcaggggtgggatagggagagatatggcaataggagccttggctaggagcccacgacaataattccaagtgagaggtggtgctgcattgaaagcaggactaacaagacctgctgacagtgtggatgtagaaaaagatagaggagacgaaggtgcatctagggttttctgcctgaggaattagaaagataaagctaaagcttatagaagatgcagcgctctggggagaaagaccagcagctcagttttgatccatctggaattaattttggcataaagtatgaggtatgtgggttaacattatttgttttttttttttccatgtagctatccaactgtcccagcatcatttattttaaaagactttcctttcccctattggattgttttggcaccttcactgaagatcaactgagcataaaattgggtctatttctaagctcttgattccattccatgacctatttgttcatctttaccccagtagacactgccttgatgattaaagcccctgttaccatgtctgttttggacatggtaaatctgagatgcctattagccaaccaagcaagcacggcccttagagagctagatatgagagcctggaattcagacgagaaaggtcagtcctagagacatacatgtagtgccatcaccatgcggatggtgttaaaagccatcagactgcaacagactgtgagagggtaccaagctagagagcatggatagagaaacccaagcactgagctgggaggtgctcctacattaagagattagtgagatgaaggactgagaagattgatcagagaagaaggaaaatcaggaaaatggtgctgtcctgaaaatccaagggaagagatgttccaaagaggagaaaactgatcagttgtcagctagcgtcaattgggatgaaaatggaccattggacagagggatgtagtgggtcatgggtgaatagataagagcagcttctatagaatggcaggggcaaaattctcatctgatcggcatgggttctaaagaaaacgggaagaaaaaattgagtgcatgaccagtcccttcaagtagagaggtggaaaagggaaggaggaaaatgaggccacgacaacatgagagaaatgacagcatttttaaaaattttttattttattttatttatttatttttgctttttagggctgcccctgcaacatatggaggttcccaggttaggggtctaatcagagctatagctgccagcctacaccacagccatagcaatgccagatctacatgacctacaccacagctcacagcaacgccggatccttaacccactgagtgaggccagagatcaaacccatatccttatggatactagtcaggttcattaccactgagccaaaatgggaaatcctgagtaatgacagcattttttaatgtgccaggaagcaaaacttgccaccccgaaatgtctctcaggcatgtggattattttgagctgaaaacgattaaggcccaaaaaacacaagaagaaatgtggaccttcccccaacagcctaaaaaatttagattgagggcctgttcccagaatagagctattgccagacttgtctacagaggctaagggctaggtgtggtggggaaaccctcagagatcagagggacgtttatgtaccaagcattgacatttccatctccatgcgaatggccttcttcccctctgtagccccaaaccaccacccccaaaatcttcttctgtctttagctgaagatggtgttgaaggtgatagtttcagccactttggcgagttcctcagttgttctgggtctttcctccggatccacattattcgactgtgtttgattttctcctgtttatctgtctcattggcacccatttcattcttagaccagcccaaagaacctagaagagtgaaggaaaatttcttccaccctgacaaatgctaaatgagaatcaccgcagtagaggaaaatgatctggtgctgcgggagatagaagagaaaatcgctggagagatgtcactgagtaggtgagatgggaaagggggggcacaggtggaggtgttgccctcagctaggaagacagacagttcacagaagagaagcgggtgtccgtggacatcttgcctcatggatgaggaaaccgaggctaagaaagactgcaaaagaaaggtaaggattgcagagaggtcgatccatgactaaaatcacagtaaccaaccccaaaccaccatgttttctcctagtctggcacgtggcaggtactgtgtaggttttcaatattattggtttgtaacagtacctattaggcctccatcccctcctctaatactaacaaaagtgtgagactggtcagtgaaaaatggtcttctttctctatgcaatctttctcaagaagatacataactttttattttatcataggcttgaagagcaaatgagaaacagcctccaacctatgacaccgtaacaaagtgtttatgatcagtgaagggcaagaaacaaaacatacacagtaaagaccctccataatattgtgggctggcccaacacaggccaggttgtaaaagctttttattctttgatagaggaatggatagtaatgtttcaacctggacagagatcatgttcactgaatccttccaaaaattcatgggtagtttgaattataaggaaaataagacttaggataaatactttgtccagatcccagagttaatgccaaaatcagttttcagactccaggcagcctgatcaagagcctaaactttaaagacacagtcccttaataactactattcacagttgcactttcagggcgcaaagactcattgaatcctacaatagaatgagtttagatatcaaatctctcagtaatagatgaggagactaaatagcgggcatgacctggtcacttaaagacagaattgagattcaaggctagtgttctttctacctgttttgtttctacaagatgtagcaatgcgctaattacagacctctcagggaaggaattcacaaccctcagcaaaaaccaaagacaaatctaagacaactaagagtgttggtttaatttggaaaaataactcactaaccaaacgcccctcttagcaccccaatgtcttccaccatcacagtgctcaggcctcaaccatgccccaatcaccccagccccagactggttattaccaagtttcatgatgactggcctgagaagatcaaaaaagcaatgacatcttacaggggactaccccgaggaccaagatagcaactgtcatagcaaccgtcacactgctttggtca

SEQ ID NO: 17 - DNA, 25 bp, Sus scrofa
ggatcaaacacgcatcctcatggac

SEQ ID NO: 18 - DNA, 22 bp, Sus scrofa
ggtgattggggcatggttgagg

SEQ ID NO: 19 - DNA, 3759 bp, Sus scrofa
ggatcaaacacgcatcctcatggacaatatgttgggttcttagcctgctgagacacaacaggaactcccctggcaccactttagaggccagagaaacagcacagataaaattccctgccctcatgaagcttatagtctagctggggagatatcataggcaagataaacacatacaaatacatcatcttaggtaataatatatactaaggagaaaattacaggggagaaagaggacaggaattgctagggtaggattataagttcagatagttcatcaggaacactgttgctgagaagataacatttaggtaaagaccgaagtagtaaggaaatggaccgtgtgcctaagtgggtaagaccattctaggcagcaggaacagcgatgaaagcactgaggtgggtgttcactgcacagagttgttcactgcacagagttgtgtggggaggggtaggtcttgcaggctcttatggtcacaggaagaattgttttactcccaccgagatgaaggttggtggattttgagcagaagaataattctgcctggtttatatataacaggatttccctgggtgctctgatgagaataatctgtcaggggtgggatagggagagatatggcaataggagccttggctaggagcccacgacaataattccaagtgagaggtggtgctgcattgaaagcaggactaacaagacctgctgacagtgtggatgtagaaaaagatagaggagacgaaggtgcatctagggttttctgcctgaggaattagaaagataaagctaaagcttatagaagatgcagcgctctggggagaaagaccagcagctcagttttgatccatctggaattaattttggcataaagtatgaggtatgtgggttaacattatttgttttttttttttccatgtagctatccaactgtcccagcatcatttattttaaaagactttcctttcccctattggattgttttggcaccttcactgaagatcaactgagcataaaattgggtctatttctaagctcttgattccattccatgacctatttgttcatctttaccccagtagacactgccttgatgattaaagcccctgttaccatgtctgttttggacatggtaaatctgagatgcctattagccaaccaagcaagcacggcccttagagagctagatatgagagcctggaattcagacgagaaaggtcagtcctagagacatacatgtagtgccatcaccatgcggatggtgttaaaagccatcagactgcaacagactgtgagagggtaccaagctagagagcatggatagagaaacccaagcactgagctgggaggtgctcctacattaagagattagtgagatgaaggactgagaagattgatcagagaagaaggaaaatcaggaaaatggtgctgtcctgaaaatccaagggaagagatgttccaaagaggagaaaactgatcagttgtcagctagcgtcaattgggatgaaaatggaccattggacagagggatgtagtgggtcatgggtgaatagataagagcagcttctatagaatggcaggggcaaaattctcatctgatcggcatgggttctaaagaaaacgggaagaaaaaattgagtgcatgaccagtcccttcaagtagagaggtggaaaagggaaggaggaaaatgaggccacgacaacatgagagaaatgacagcatttttaaaaattttttattttattttatttatttatttttgctttttagggctgcccctgcaacatatggaggttcccaggttaggggtctaatcagagctatagctgccagcctacaccacagccatagcaatgccagatctacatgacctacaccacagctcacagcaacgccggatccttaacccactgagtgaggccagagatcaaacccatatccttatggatactagtcaggttcattaccactgagccaaaatgggaaatcctgagtaatgacagcattttttaatgtgccaggaagcaaaacttgccaccccgaaatgtctctcaggcatgtggattattttgagctgaaaacgattaaggcccaaaaaacacaagaagaaatgtggaccttcccccaacagcctaaaaaatttagattgagggcctgttcccagaatagagctattgccagacttgtctacagaggctaagggctaggtgtggtggggaaaccctcagagatcagagggacgtttatgtaccaagcattgacatttccatctccatgcgaatggccttcttcccctctgtagccccaaaccaccacccccaaaatcttcttctgtctttagctgaagatggtgttgaaggtgatagtttcagccactttggcgagttcctcagttgttctgggtctttcctcctgatccacattattcgactgtgtttgattttctcctgtttatctgtctcattggcacccatttcattcttagaccagcccaaagaacctagaagagtgaaggaaaatttcttccaccctgacaaatgctaaatgagaatcaccgcagtagaggaaaatgatctggtgctgcgggagatagaagagaaaatcgctggagagatgtcactgagtaggtgagatgggaaaggggtgacacaggtggaggtgttgccctcagctaggaagacagacagttcacagaagagaagcgggtgtccgtggacatcttgcctcatggatgaggaaaccgaggctaagaaagactgcaaaagaaaggtaaggattgcagagaggtcgatccatgactaaaatcacagtaaccaaccccaaaccaccatgttttctcctagtctggcacgtggcaggtactgtgtaggttttcaatattattggtttgtaacagtacctattaggcctccatcccctcctctaatactaacaaaagtgtgagactggtcagtgaaaaatggtcttctttctctatgaatctttctcaagaagatacataactttttattttatcataggcttgaagagcaaatgagaaacagcctccaacctatgacaccgtaacaaaatgtttatgatcagtgaagggcaagaaacaaaacatacacagtaaagaccctccataatattgtgggtggcccaacacaggccaggttgtaaaagctttttattctttgatagaggaatggatagtaatgtttcaacctggacagagatcatgttcactgaatccttccaaaaattcatgggtagtttgaattataaggaaaataagacttaggataaatactttgtccaagatcccagagttaatgccaaaatcagttttcagactccaggcagcctgatcaagagcctaaactttaaagacacagtcccttaataactactattcacagttgcactttcagggcgcaaagactcattgaatcctacaatagaatgagtttagatatcaaatctctcagtaatagatgaggagactaaatagcgggcatgacctggtcacttaaagacagaattgagattcaaggctagtgttctttctacctgttttgtttctacaagatgtagcaatgcgctaattacagacctctcagggaaggaattcacaaccctcagcaaaaaccaaagacaaatctaagacaactaagagtgttggtttaatttggaaaaataactcactaaccaaacgcccctcttagcaccccaatgtcttccaccatcacagtgctcaggcctcaaccatgccccaatcacc

SEQ ID NO: 20 - DNA, 9010 bp, Sus scrofa
ctcaaacgtaagtggctttttccgactgattctttgctgtttctaattgttggttggctttttgtccatttttcagtgttttcatcgaattagttgtcagggaccaaacaaattgccttcccagattaggtaccagggaggggacattgctgcatgggagaccagagggtggctaatttttaacgtttccaagccaaaataactggggaagggggcttgctgtcctgtgagggtaggtttttatagaagtggaagttaaggggaaatcgctatggttcacttttggctcggggaccaaagtggagcccaaaattgagtacattttccatcaattatttgtgagatttttgtcctgttgtgtcatttgtgcaagtttttgacattttggttgaatgagccattcccagggacccaaaaggatgagaccgaaaagtagaaaagagccaacttttaagctgagcagacagaccgaattgttgagtttgtgaggagagtagggtttgtagggagaaaggggaacagatcgctggctttttctctgaattagcctttctcatgggactggcttcagagggggtttttgatgagggaagtgttctagagccttaactgtgggttgtgttcggtagcgggaccaagctggaaatcaaacgtaagtgcacttttctactcctttttctttcttatacgggtgtgaaattggggacttttcatgtttggagtatgagttgaggtcagttctgaagagagtgggactcatccaaaaatctgaggagtaagggtcagaacagagttgtctcatggaagaacaaagacctagttagttgatgaggcagctaaatgagtcagttgacttgggatccaaatggccagacttcgtctgtaaccaacaatctaatgagatgtagcagcaaaaagagatttccattgaggggaaagtaaaattgttaatattgtggatcacctttggtgaagggacatccgtggagattgaacgtaagtattttttctctactaccttctgaaatttgtctaaatgccagtgttgacttttagaggcttaagtgtcagttttgtgaaaaatgggtaaacaagagcatttcatatttattatcagtttcaaaagttaaactcagctccaaaaatgaatttgtagacaaaaagattaatttaagccaaattgaatgattcaaaggaaaaaaaaattagtgtagatgaaaaaggaattcttacagctccaaagagcaaaagcgaattaattttctttgaactttgccaaatcttgtaaatgatttttgttctttacaatttaaaaaggttagagaaatgtatttcttagtctgttttctctcttctgtctgataaattattatatgagataaaaatgaaaattaataggatgtgctaaaaaatcagtaagaagttagaaaaatatatgtttatgttaaagttgccacttaattgagaatcagaagcaatgttatttttaaagtctaaaatgagagataaactgtcaatacttaaattctgcagagattctatatcttgacagatatctcctttttcaaaaatccaatttctatggtagactaaatttgaaatgatcttcctcataatggagggaaaagatggactgaccccaaaagctcagatttaagaaaacctgtttaaggaaagaaaataaaagaactgcattttttaaaggcccatgaatttgtagaaaaataggaaatattttaataagtgtattcttttattttcctgttattacttgatggtgtttttataccgccaaggaggccgtggcaccgtcagtgtgatctgtagaccccatggcggccttttttcgcgattgaatgaccttggcggtgggtccccagggctctggtggcagcgcaccagccgctaaaagccgctaaaaactgccgctaaaggccacagcaaccccgcgaccgcccgttcaactgtgctgacacagtgatacagataatgtcgctaacagaggagaatagaaatatgacgggcacacgctaatgtggggaaaagagggagaagcctgatttttattttttagagattctagagataaaattcccagtattatatccttttaataaaaaatttctattaggagattataaagaatttaaagctatttttttaagtggggtgtaattctttcagtagtctcttgtcaaatggatttaagtaatagaggcttaatccaaatgagagaaatagacgcataaccctttcaaggcaaaagctacaagagcaaaaattgaacacagcagccagccatctagccactcagattttgatcagttttactgagtttgaagtaaatatcatgaaggtataattgctgataaaaaaataagatacaggtgtgacacatctttaagtttcagaaatttaatggcttcagtaggattatatttcacgtatacaaagtatctaagcagataaaaatgccattaatggaaacttaatagaaatatatttttaaattccttcattctgtgacagaaattttctaatctgggtcttttaatcacctaccctttgaaagagtttagtaatttgctatttgccatcgctgtttactccagctaatttcaaaagtgatacttgagaaagattatttttggtttgcaaccacctggcaggactattttagggccattttaaaactcttttcaaactaagtattttaaactgttctaaaccatttagggccttttaaaaatcttttcatgaatttcaaacttcgttaaaagttattaaggtgtctggcaagaacttccttatcaaatatgctaatagtttaatctgttaatgcaggatataaaattaaagtgatcaaggcttgacccaaacaggagtatcttcatagcatatttcccctcctttttttctagaattcatatgattttgctgccaaggctattttatataatctctggaaaaaaaatagtaatgaaggttaaaagagaagaaaatatcagaacattaagaattcggtattttactaactgcttggttaacatgaaggtttttattttattaaggtttctatctttataaaaatctgttcccttttctgctgatttctccaagcaaaagattcttgatttgttttttaactcttactctcccacccaagggcctgaatgcccacaaaggggacttccaggaggccatctggcagctgctcaccgtcagaagtgaagccagccagttcctcctgggcaggtggccaaaattacagttgacccctcctggtctggctgaaccttgccccatatggtgacagccatctggccagggcccaggtctccctctgaagcctttgggaggagagggagagtggctggcccgatcacagatgcggaaggggctgactcctcaaccggggtgcagactctgcagggtgggtctgggcccaacacacccaaagcacgcccaggaaggaaaggcagcttggtatcactgcccagagctaggagaggcaccgggaaaatgatctgtccaagacccgttcttgcttctaaactccgagggggtcagatgaagtggttttgtttcttggcctgaagcatcgtgttccctgcaagaagcggggaacacagaggaaggagagaaaagatgaactgaacaaagcatgcaaggcaaaaaagggggtctagccgcggtctaggaagctttctagggtacctctagggatcccggcgcgccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttggctccttcgctttctgggctcagaggctgggaaggggtgggtccgggggcgggctcaggggcgggctcaggggcggggcgggcgcccgaaggtcctccggaagcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcaatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggatcgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggggcgcgcccctcgagcggccgccagtgtgatggatatctgcagaattcgcccttggatcaaacacgcatcctcatggacaatatgttgggttcttagcctgctgagacacaacaggaactcccctggcaccactttagaggccagagaaacagcacagataaaattccctgccctcatgaagcttatagtctagctggggagatatcataggcaagataaacacatacaaatacatcatcttaggtaataatatatactaaggagaaaattacaggggagaaagaggacaggaattgctagggtaggattataagttcagatagttcatcaggaacactgttgctgagaagataacatttaggtaaagaccgaagtagtaaggaaatggaccgtgtgcctaagtgggtaagaccattctaggcagcaggaacagcgatgaaagcactgaggtgggtgttcactgcacagagttgttcactgcacagagttgtgtggggaggggtaggtcttgcaggctcttatggtcacaggaagaattgttttactcccaccgagatgaaggttggtggattttgagcagaagaataattctgcctggtttatatataacaggatttccctgggtgctctgatgagaataatctgtcaggggtgggatagggagagatatggcaataggagccttggctaggagcccacgacaataattccaagtgagaggtggtgctgcattgaaagcaggactaacaagacctgctgacagtgtggatgtagaaaaagatagaggagacgaaggtgcatctagggttttctgcctgaggaattagaaagataaagctaaagcttatagaagatgcagcgctctggggagaaagaccagcagctcagttttgatccatctggaattaattttggcataaagtatgaggtatgtgggttaacattatttgttttttttttttccatgtagctatccaactgtcccagcatcatttattttaaaagactttcctttcccctattggattgttttggcaccttcactgaagatcaactgagcataaaattgggtctatttctaagctcttgattccattccatgacctatttgttcatctttaccccagtagacactgccttgatgattaaagcccctgttaccatgtctgttttggacatggtaaatctgagatgcctattagccaaccaagcaagcacggcccttagagagctagatatgagagcctggaattcagacgagaaaggtcagtcctagagacatacatgtagtgccatcaccatgcggatggtgttaaaagccatcagactgcaacagactgtgagagggtaccaagctagagagcatggatagagaaacccaagcactgagctgggaggtgctcctacattaagagattagtgagatgaaggactgagaagattgatcagagaagaaggaaaatcaggaaaatggtgctgtcctgaaaatccaagggaagagatgttccaaagaggagaaaactgatcagttgtcagctagcgtcaattgggatgaaaatggaccattggacagagggatgtagtgggtcatgggtgaatagataagagcagcttctatagaatggcaggggcaaaattctcatctgatcggcatgggttctaaagaaaacgggaagaaaaaattgagtgcatgaccagtcccttcaagtagagaggtggaaaagggaaggaggaaaatgaggccacgacaacatgagagaaatgacagcatttttaaaaattttttattttattttatttatttatttttgctttttagggctgcccctgcaacatatggaggttcccaggttaggggtctaatcagagctatagctgccagcctacaccacagccatagcaatgccagatctacatgacctacaccacagctcacagcaacgccggatccttaacccactgagtgaggccagagatcaaacccatatccttatggatactagtcaggttcattaccactgagccaaaatgggaaatcctgagtaatgacagcattttttaatgtgccaggaagcaaaacttgccaccccgaaatgtctctcaggcatgtggattattttgagctgaaaacgattaaggcccaaaaaacacaagaagaaatgtggaccttcccccaacagcctaaaaaatttagattgagggcctgttcccagaatagagctattgccagacttgtctacagaggctaagggctaggtgtggtggggaaaccctcagagatcagagggacgtttatgtaccaagcattgacatttccatctccatgcgaatggccttcttcccctctgtagccccaaaccaccacccccaaaatcttcttctgtctttagctgaagatggtgttgaaggtgatagtttcagccactttggcgagttcctcagttgttctgggtctttcctcctgatccacattattcgactgtgtttgattttctcctgtttatctgtctcattggcacccatttcattcttagaccagcccaaagaacctagaagagtgaaggaaaatttcttccaccctgacaaatgctaaatgagaatcaccgcagtagaggaaaatgatctggtgctgcgggagatagaagagaaaatcgctggagagatgtcactgagtaggtgagatgggaaaggggtgacacaggtggaggtgttgccctcagctaggaagacagacagttcacagaagagaagcgggtgtccgtggacatcttgcctcatggatgaggaaaccgaggctaagaaagactgcaaaagaaaggtaaggattgcagagaggtcgatccatgactaaaatcacagtaaccaaccccaaaccaccatgttttctcctagtctggcacgtggcaggtactgtgtaggttttcaatattattggtttgtaacagtacctattaggcctccatcccctcctctaatactaacaaaagtgtgagactggtcagtgaaaaatggtcttctttctctatgaatctttctcaagaagatacataactttttattttatcataggcttgaagagcaaatgagaaacagcctccaacctatgacaccgtaacaaaatgtttatgatcagtgaagggcaagaaacaaaacatacacagtaaagaccctccataatattgtgggtggcccaacacaggccaggttgtaaaagctttttattctttgatagaggaatggatagtaatgtttcaacctggacagagatcatgttcactgaatccttccaaaaattcatgggtagtttgaattataaggaaaataagacttaggataaatactttgtccaagatcccagagttaatgccaaaatcagttttcagactccaggcagcctgatcaagagcctaaactttaaagacacagtcccttaataactactattcacagttgcactttcagggcgcaaagactcattgaatcctacaatagaatgagtttagatatcaaatctctcagtaatagatgaggagactaaatagcgggcatgacctggtcacttaaagacagaattgagattcaaggctagtgttctttctacctgttttgtttctacaagatgtagcaatgcgctaattacagacctctcagggaaggaa

SEQ ID NO: 21 - DNA, 21 bp, Sus scrofa
cgaacccctgtgtatatagtt

SEQ ID NO: 22 - DNA, 21 bp, Sus scrofa
gagatgaggaagaggagaaca

SEQ ID NO: 23 - DNA, 23 bp, Sus scrofa
gcattgtctgagtaggtgtcatt

SEQ ID NO: 24 - DNA, 22 bp, Sus scrofa
cgcttcttgcagggaacacgat

SEQ ID NO: 25 - DNA, 6772 bp, Sus scrofa
gcacatggtaggcaaaggactttgcttctcccagcacatctttctgcagagatccatggaaacaagactcaactccaaagcagcaaagaagcagcaagttctcaagtgatctcctctgactccctcctcccaggctaatgaagccatgttgcccctgggggattaagggcaggtgtccattgtggcacccagcccgaagacaagcaatttgatcaggttctgagcactcctgaatgtggactctggaattttctcctcaccttgtggcatatcagcttaagtcaagtacaagtgacaaacaacataatcctaagaagagaggaatcaagctgaagtcaaaggatcactgccttggattctactgtgaatgatgacctggaaaatatcctgaacaacagcttcagggtgatcatcagagacaaaagttccagagccaggtagggaaaccctcaagccttgcaaagagcaaaatcatgccattgggttcttaacctgctgagtgatttactatatgttactgtgggaggcaaagcgctcaaatagcctgggtaagtatgtcaaataaaaagcaaaagtggtgtttcttgaaatgttagacctgaggaaggaatattgataacttaccaataattttcagaatgatttatagatgtgcacttagtcagtgtctctccaccccgcacctgacaagcagtttagaatttattctaagaatctaggtttgctgggggctacatgggaatcagcttcagtgaagagtttgttggaatgattcactaaattttctatttccagcataaatccaagaacctctcagactagtttattgacactgcttttcctccataatccatctcatctccgtccatcatggacactttgtagaatgacaggtcctggcagagactcacagatgcttctgaaacatcctttgccttcaaagaatgaacagcacacatactaaggatctcagtgatccacaaattagtttttgccacaatggttcttatgataaaagtctttcattaacagcaaattgttttataatagttgttctgctttataataattgcatgcttcactttcttttcttttctttttttttctttttttgctttttagtgccgcaggtgcagcatatgaaatttcccaggctaggggtcaaatcagaactacacctactggcctacgccacagccacagcaactcaggatctaagccatgtcggtgacctacactacagctcatggcaatgccagatccttaacccaatgagcgaggccagggatcgaacccatgtcctcatggatactagtcaggctcattatccgctgagccataacaggaactcccgagtttgctttttatcaaaattggtacagccttattgtttctgaaaaccacaaaatgaatgtattcacataattttaaaaggttaaataatttatgatatacaagacaatagaaagagaaaacgtcattgcctctttcttccacgacaacacgcctccttaattgatttgaagaaataactactgagcatggtttagtgtacttctttcagcaattagcctgtattcatagccatacatattcaattaaaatgagatcatgatatcacacaatacataccatacagcctatagggatttttacaatcatcttccacatgactacataaaaacctacctaaaaaaaaaaaaaaccctacttcatcctcctattggctgctttgtgctccattaaaaagctctatcataattaggttatgatgaggatttccattttctacctttcaagcaacatttcaatgcacagtcttatatacacatttgagcctacttttctttttctttctttttttggttttttttttttttttttttttggtctttttgtcttttctaaggctgcatatggaggttcccaggctagctgtctaatcagaactatagctgctggcctacgccacatccacagcaatacaagatctgagccatgtctgcaacttacaccacagctcacagcaacggtggatccttaaaccactgagcaaggccagggatcaaacccataacttcatggctcctagttggatttgttaaccactgagccatgatggcaactcctgagcctacttttctaatcatttccaaccctaggacacttttttaagtttcatttttctccccccaccccctgttttctgaagtgtgtttgcttccactgggtgacttcactcccaggatctcatctgcaggatactgcagctaagtgtatgagctctgaatttgaatcccaactctgccactcaaagggataggagtttccgatgtggcccaatgggatcagtggcatctctgcagtgccaggacgcaggttccatccctggcccagcacagtgggttaagaatctggcattgctgcagctgaggcatagatttcaattgtgcctcagatctgatccttggcccaaggactgcatatgcctcagggcaaccaaaaaagagaaaaggggggtgatagcattagtttctagatttgggggataattaaataaagtgatccatgtacaatgtatggcattttgtaaatgctcaacaaatttcaactattatggagttcccatcatggctcagtggaagggaatctgattagcatccatgaggacacaggtccaaccccgaccttgctcagtgggcattgctgtgagctgtggcatgggttacagacgaagctcggatctggcattgctgtggctgtggtgtaagccagcaactacagctctcattcagcccctagcctgggaacctccatatgcctaaaagacaaaaaataaaatttaaattaaaaataaagaaatgttaactattatgattggtactgcttgcattactgcaaagaaagtcactttctatactctttaatatcttagttgactgtgtgctcagtgaactattttggacacttaatttccactctcttctatctccaacttgacaactctctttcctctcttctggtgagatccactgctgactttgctctttaaggcaactagaaaagtgctcagtgacaaaatcaaagaaagttaccttaatcttcagaattacaatcttaagttctcttgtaaagcttactatttcagtggttagtattattccttggtcccttacaacttatcagctctgatctattgctgattttcaactatttattgttggagttttttccttttttccctgttcattctgcaaatgtttgctgagcatttgtcaagtgaagatactggactgggccttccaaatataagacaatgaaacatcgggagttctcattatggtgcagcagaaacgaatccaactaggaaatgtgaggttgcaggttcgatccctgcccttgctcagtgggttaaggatccagcattaccgtgagctgtggtgtaggttgcagacgtggctcagatcctgcgttgctgtggctgtggcataggctggcagctctagctctgattcgaccgctagcctgggaacctccatgcgccccgagtgcagcccttaaaaagcaaaaaaaaaagaaagaaagaaaaagacaatgaaacatcaaacagctaacaatccagtagggtagaaagaatctggcaacagataagagcgattaaatgttctaggtccagtgaccttgcctctgtgctctacacagtcgtgccacttgctgagggagaaggtctctcttgagttgagtcctgaaagacattagttgttcacaaactaatgccagtgagtgaaggtgtttccaagcagagggagagtttggtaaaaagctggaagtcacagaaagactctaaagagtttaggatggtgggagcaacatacgctgagatggggctggaaggttaagagggaaacaactatagtaagtgaagctggactcacagcaaagtgaggacctcagcatccttgatggggttaccatggaaacaccaaggcacaccttgatttccaaaacagcaggcacctgattcagcccaatgtgacatggtgggtacccctctagctctacctgttctgtgacaactgacaaccaacgaagttaagtctggattttctactctgctgatccttgtttttgtttcacacgtcatctatagcttcatgccaaaatagagttcaaggtaagacgcgggccttggtttgatatacatgtagtctatcttgtttgagacaatatggtggcaaggaagaggttcaaacaggaaaatactctctaattatgattaactgagaaaagctaaagagtcccataatgacactgaatgaagttcatcatttgcaaaagccttcccccccccccaggagactataaaaaagtgcaattttttaaatgaacttatttacaaaacagaaatagactcacagacataggaaacgaacagatggttaccaagggtgaaagggagtaggagggataaataaggagtctggggttagcagatacaccccagtgtacacaaaataaacaacagggacctactatatagcacagggaactatatgcagtagcttacaataacctataatggaaaagaatgtgaaaaagaatatatgtatgcgtgtgtgtgtaactgaatcactttgctgtaacctgaatctaacataacattgtaaatcaactacagttttttttttttttaagtgcagggttttggtgttttttttttttcatttttgtttttgtttttgttttttgctttttagggccacacccagacatatgggggttcccaggctaggggtctaattagagctacagttgccggcttgcaccacagccacagcaacatcagatccgagccgcacttgcgacttacaccacagctcatggcaataccagatccttaacccactgagcaaggcccagggatcgtacccgcaacctcatggttcctagtcagattcatttctgctgcgctacaatgggaactccaagtgcagttttttgtaatgtgcttgtctttctttgtaattcatattcatcctacttcccaataaataaataaatacataaataataaacataccattgtaaatcaactacaattttttttaaatgcagggtttttgttttttgttttttgttttgtctttttgccttttctagggccgctcccatggcatatggaggttcccaggctaggggtcgaatcggagctgtagccaccggcctacgccagagccacagcaacgcgggatccgagccgcgtctgcaacctacaccacagctcacggcaacgccggatcgttaacccactgagcaagggcagggatcgaacctgcaacctcatggttcctagtcagattcgttaactactgagccacaacggaaactcctaaagtgcagtttttaaatgtgcttgtctttctttgtaatttacactcaacctacttcccaataaataaataaataaacaaataaatcatagacatggttgaattctaaaggaagggaccatcaggccttagacagaaatacgtcatcttctagtattttaaaacacactaaagaagacaaacatgctctgccagagaagcccagggcctccacagctgcttgcaaagggagttaggcttcagtagctgacccaaggctctgttcctcttcagggaaaagggtttttgttcagtgagacagcagacagctgtcactgtggtggacgttcggccaaggaaccaagctggaactcaaacgtaagtcaatccaaacgttccttccttggctgtctgtgtcttacggtctctgtggctctgaaatgattcatgtgctgactctctgaaaccagactgacattctccagggcaaaactaaagcctgtcatcaaaccggaaaactgagggcacattttctgggcagaactaagagtcaggcactgggtgaggaaaaacttgttagaatgatagtttcagaaacttactgggaagcaaagcccatgttctgaacagagctctgctcaagggtcaggaggggaaccagtttttgtacaggagggaagttgagacgaacccctgtgtatatggtttcggcgcggggaccaagctggagctcaaacgtaagtggctttttccgactgattctttgctgtttctaattgttggttggctttttgtccatttttcagtgttttcatcgaattagttgtcagggaccaaacaaattgccttcccagattaggtaccagggaggggacattgctgcatgggagaccagagggtggctaatttttaacgtttccaagccaaaataactggggaagggggcttgctgtcctgtgagggtaggtttttatagaagtggaagttaaggggaaatcgctatggttcacttttggctcggggaccaaagtggagcccaaaattgagtacattttccatcaattatttgtgagatttttgtcctgttgtgtcatttgtgcaagtttttgacattttggttgaatgagccattcccagggacccaaaaggatgagaccgaaaagtagaaaagagccaacttttaagctgagcagacagaccgaattgttgagtttgtgaggagagtagggtttgtagggagaaaggggaacagatcgctggctttttctctgaattagcctttctcatgggactggcttcagagggggtttttgatgagggaagtgttctagagccttaactgtgggttgtgttcggtagcgggaccaagctggaaatcaaacgtaagtgcacttttctactcc

SEQ ID NO: 26 - DNA, 22 bp, Sus scrofa
ccttcctcctgcacctgtcaac

SEQ ID NO: 27 - DNA, 22 bp, Sus scrofa
tagacacaccagggtggccttg

SEQ ID NO: 28 - DNA, 5918 bp, Sus scrofa
ccttcctcctgcacctgtcaactcccaataaaccgtcctccttgtcattcagaaatcatgctctccgctcacttgtgtctacccattttcgggcttgcatggggtcatcctcgaaggtggagagagtcccccttggccttggggaagtcgaggggggcggggggaggcctgaggcatgtgccagcgaggggggtcacctccacgcccctgaggaccttctagaaccaggggcgtggggccaccgcctgagtggaaggctgtccacttttcccccgggcccccaggctccctcctccgtgtggaccttgtccacctctgactggcccagccactcatgcattgtttccccgaaaccccaggacgatagctcagcacgcgacagtgtccccctctgagggcctctgtccatttcaggacgacccgcatgtacagcgtgaccactctgctcacgcccactcaccacgtcctagagccccacccccagccccatccttaggggcacagccagctccgaccgccccggggacaccaccctctgccccttccccaggccctccctgtcacacgcaccacagggccctccgtcccgagaccctgctccctcatccctcggtcccctcaggtagccttccacccgcgtgtgtcccgaggtcccagatgcagcaaggcccctgggacaacgccagatctctgctctccccgacccctcagaagccagcccacgcctggccccaccaccactgcctaacgtccaagtgtccataggcctcgggacctccaagtccaggttctgcctctgggattccgccatgggtctgcctgggaaatgatgcacttggaggagctcagcatgggatgcgggaccttgtctctaggcgctccctcaggatcccacagctgccctgtgagacacacacacacacacacacacacacacacacacacacacacacacaaacacgcatgcacgcacgccggcacacacgctattgcagagatggccacggtagctgtgcctcgaggccgagtggagtgtctagaactctcgggggtcccctctgcagacgacactgctccatcccccccgtgccctgaagggctcctcactctcccatcaggatctctccaagctgctgacctggagaggaaggggcctgggacaggcggggacactcagacctccctgctgcccctcctctgcctgggcttggacggctccccccttcccacgggtgaaggtgcaggtggggagagggcacccccctcagcctcccagacccagaccagcccccgtggcaggggcagcctgtgagcctccagccagatgcaggtggcctggggtggggggtggagggggcgggaggtttatgtttgaggctgtatcactgtgtaatattttcggcggtgggacccatctgaccgtcctcggtgagtctccccttttctctcctccttggggatccgagtgaaatctgggtcgatcttctctccgttctcctccgactggggctgaggtctgaacctcggtggggtccgaagaggaggcccctaggccaggctcctcagcccctccagcccgaccggccctcttgacacagggtccagctaagggcagacatggaggctgctagtccagggccaggctctgagacccaagggcgctgcccaaggaacccttgccccagggaccctgggagcaaagctcctcactcagagcctgcagccctggggtctgaggacaaggagggactgaggactgggcgtggggagttcaggcggggacaccaggtccagggaggtgacaaaggcgctgggagggggcggacggtgccggggactcctcctgggccctgtgggctcggggtccttgtgaggaccctgagggactgaggggcccctgggcctagggacttgcagtgagggaggcagggagtgtcccttgagaacgtggcctccgcgggctgggtccccctcgtgctcccagccgggaggacaccccagagcaagcgccccaggtgggcggggagggtctcctcacaggggcagctgacagatagaggcccccgccaggcagatgcttgatcctggcagttatactgggttcgcacaactttccctgaacaaggggccctccgaacagacacagacgcaacccagtcgacccaggctcagcacagaaaatgcactgacacccaaaaccctcatctgggggcctggccggcatcccgccccaggacccaaggcccctgccccctggcagccctggacacggtcctctgtgggcggtggggtcggggctgtggtgacggtggcatcggggagcctgtgccccctccctgaaagggcggagaggctcaagaggggacagaaatgtcctcccctaggaagacctcggacgggggcgggggggtggtctccgacagacagatgcccgggaccgacagacctgccgagggaagagggcacctcggtcgggttaggctccaggcagcacgagggagcgaggctgggagggtgaggacatgggagcctgaggaggagctggagacttcagcaggcccccagctccgggcttcgggctctgagatgctcggacgcaaggtgagtgaccccacctgtggctgacctgacctcagggggacaaggctcagcctgggactctgtgtccccatcgcctgcacaggggattcccctgatggacactgagccaacgacctcccgtctctccccgacccccaggtcagcccaaggccactcccacggtcaacctcttcccgccctcctctgaggagctcggcaccaacaaggccaccctggtgtgtctaataagtgacttctacccgggcgccgtgacggtgacctggaaggcaggcggcaccaccgtcacccagggcgtggagaccaccaagccctcgaaacagagcaacaacaagtacgcggccagcagctacctggccctgtccgccagtgactggaaatcttccagcggcttcacctgccaggtcacccacgaggggaccattgtggagaagacagtgacgccctccgagtgcgcctaggtccctgggcccccaccctcaggggcctggagccacaggacccccgcgagggtctccccgcgaccctggtccagcccagcccttcctcctgcacctgtcaactcccaataaaccgtcctccttgtcattcagaaatcatgctctccgctcacttgtgtctacccattttcgggcttgcatggggtcatcctcgaaggtggagagagtcccccttggccttggggaaatcgaggggggcggggggaggcctgaggcatgtgccagcgaggggggtcacctccacgcccctgaggaccttctagaaccaggggcgtggggccaccgccagagtggaaggctgtccacttttcccccgggcccccaggctccctcctccgtgtggaccttgtccacctctgactggcccagccactcatgcattgtttccccgaaaccccaggacgatagctcagcacgcgacagtgtccccctctgagggcctctgtccatttcaggacgacccgcatgtacagcgtgaccactctgctcacgcccactcaccacgtcctagagccccacccccagccccatccttaggggcacagccagctccgaccgccccggggacaccaccctctgccccttccccaggccctccctgtcacacgcaccacagggccctccgtcccgagaccctgctccctcatccctcggtcccctcaggtagccttccacccgcgtgtgtcccgaggtcccagatgcagcaaggcccctgggacaacgccagatctctgctctccccgaccctcagaagccagcccacgcctggcccaccaccactgcctaacgtccaagtgtccataggctcgggacctccaagtccaggttctgcctctgggattccgccatgggtctgcctggaatgatgcacttggaggagctcagcatgggatgcggaacttgtctagcgctcctcagatccacagctgcctgtgagacacacacacacacacacacacaccaaacacgcatgcacgcacgccggcacacacgctattacagagatggccacggtagctgtgcctcgaggccgagtggagtgtctagaactctcgggggtcccctctgcagacgacactgctccatcccccccgtgccctgaagggctcctcactctcccatcaggatctctccaagctgctgacctggagaggaaggggcctgggacaggcggggacactcagacctccctgctgcccctcctctgcctgggcttggacggctccccccttcccacgggtgaaggtgcaggtggggagagggcacccccctcaccctcccagacccagaccagcccccgtggcaggggcagcctgtgagcctccagccagatgcaggtggcctggggtggggggtggagggggcgggaggtttatgtttgaggctgtattcatctgtgtaatattttcggcggtgggacccatctgaccgtcctcggtgagtctccccttttctttcctccttggggatccgagtgaaatctgggtcgatcttctctccgttctcctccgactggggctgaggtctgaacctcggtggggtccgaagaggaggcccctaggccggctcctcagcccctccagcccgacccgccctcttgacacagggtccagctaagggcagacatggctgctagtccagggccaggctctgagacccaagggcgctgcccaaggaacccttgccccagggaccctgggagcaaagctcctcactcagagcctgcagccctggggtctgaggacaaggagggactgaggactgggcgtggggagttcaggcggggacaccgggtccagggaggtgacaaaggcgctgggagggggcggacggtgccggagactcctcctgggccctgtgggctcgtggtccttgtgaggaccctgagggctgaggggcccctgggcctagggacttgcagtgagggaggcagggagtgtcccttgagaacgtggcctccgcgggctgggtccccctcgtgctcccagcagggaggacaccccagagcaagcgccccaggtgggcggggagggtctcctcacaggggcagctgacagatagacggcccccgccagacagatgcttgatcctggtcagtactgggttcgccacttccctgaacaggggccctccgaacagacacagacgcagaccaggctcagcacagaaaatgcactgacacccaaaaccctcatctgggggcctggccggcatcccgccccaggacccaaggcccctgccccctggcagccctggacacggtcctctgtgggcggtggggtcggggctgtggtgacggtggcatcggggagcctgtgccccctccctgaaagggcggagaggctcaagaggggacagaaatgtcctcccctaggaagacctcggacgggggcgggggggtggtctccgacagacagatgcccgggaccgacagacctgccgagggaagagggcacctcggtcgggttaggctccaggcagcacgagggagcgaggctgggagggtgaggacatgggagcctgaggaggagctggagacttcagcaggcccccagctccgggcttcgggctctgagatgctcggacgcaaggtgagtgaccccacctgtggctgacctgacctgacctcagggggacaaggctcagcctgggactctgtgtccccatcgcctgcacaggggattcccctgatggacactgagccaacgacctcccgtctctccccgacccccaggtcagcccaaggccactcccacggtcaacctcttcccgccctcctctgaggagctcggcaccaacaaggccaccctggtgtgtcta

SEQ ID NO: 29 - DNA, 11051 bp, Sus scrofa
tctagaagacgctggagagaggccagacttcctcggaacagctcaaagagctctgtcaaagccagatcccatcacacgtgggcaccaataggccatgccagcctccaagggccgaactgggttctccacggcgcacatgaagcctgcagcctggcttatcctcttccgtggtgaagaggcaggcccgggactggacgaggggctagcagggtgtggtaggcaccttgcgccccccaccccggcaggaaccagagaccctggggctgagagtgagcctccaaacaggatgccccacccttcaggccacctttcaatccagctacactccacctgccattctcctctgggcacagggcccagcccctggatcttggccttggctcgacttgcacccacgcgcacacacacacttcctaacgtgctgtccgctcacccctccccagcgtggtccatgggcagcacggcagtgcgcgtccggcggtagtgagtgcagaggtcccttcccctcccccaggagccccaggggtgtgtgcagatctgggggctcctgtcccttacaccttcatgcccctcccctcatacccaccctccaggcgggaggcagcgagacctttgcccagggactcagccaacgggcacacgggaggccagccctcagcagctggctcccaaagaggaggtgggaggtaggtccacagctgccacagagagaaaccctgacggaccccacaggggccacgccagccggaaccagctccctcgtgggtgagcaatggccagggccccgccggccaccacggctggccttgcgccagctgagaactcacgtccagtgcagggagactcaagacagcctgtgcacacagcctcggatctgctcccatttcaagcagaaaaaggaaaccgtgcaggcagccctcagcatttcaaggattgtagcagcggccaactattcgtcggcagtggccgattagaatgaccgtggagaagggcggaagggtctctcgtgggctctgcggccaacaggccctggctccacctgcccgctgccagcccgaggggcttgggccgagccaggaaccacagtgctcaccgggaccacagtgactgaccaaactcccggccagagcagccccaggccagccgggctctcgccctggaggactcaccatcagatgcacaagggggcgagtgtggaagagacgtgtcgcccgggccatttgggaaggcgaagggaccttccaggtggacaggaggtgggacgcactccaggcaagggactgggtccccaaggcctggggaaggggtactggcttgggggttagcctggccagggaacggggagcggggcggggggctgagcagggaggacctgacctcgtgggagcgaggcaagtcaggcttcaggcagcagccgcacatcccagaccaggaggctgaggcaggaggggcttgcagcggggcgggggcctgcctggctccgggggctcctgggggacgctggctcttgtttccgtgtcccgcagcacagggccagctcgctgggcctatgcttaccttgatgtctggggccggggcgtcagggtcgtcgtctcctcaggggagagtcccctgaggctacgctggggggggactatggcagctccaccaggggcctggggaccaggggcctggaccaggctgcagcccggaggacgggcagggctctggctctccagcatctggccctcggaaatggcagaacccctggcgggtgagcgagctgagagcgggtcagacagacaggggccggccggaaaggagaagttgggggcagagcccgccaggggccaggcccaaggttctgtgtgccagggcctgggtgggcacattggtgtggccatggctacttagattcgtggggccagggcatcctggtcaccgtctcctcaggtgagcctggtgtctgatgtccagctaggcgctggtgggccgcgggtgggcctgtctcaggctagggcaggggctgggatgtgtatttgtcaaggaggggcaacagggtgcagactgtgcccctggaaacttgaccactggggcaggggcgtcctggtcacgtctcctcaggtaagacggccctgtgcccctctctcgcgggactggaaaaggaattttccaagattccttggtctgtgtggggccctctggggcccccgggggtggctcccctcctgcccagatggggcctcggcctgtggagcacgggctgggcacacagctcgagtctagggccacagaggcccgggctcagggctctgtgtggcccggcgactggcagggggctcgggtttttggacaccccctaatgggggccacagcactgtgaccatcttcacagctggggccgaggagtcgaggtcaccgtctcctcaggtgagtcctcgtcagccctctctcactctctggggggttttgctgcattttgtgggggaaagaggatgcctgggtctcaggtctaaaggtctagggccagcgccggggcccaggaaggggccgaggggccaggctcggctcggccaggagcagagcttccagacatctcgcctcctggcggctgcagtcaggcctttggccgggggggtctcagcaccaccaggcctcttggctcccgaggtccccggccccggctgcctcaccaggcaccgtgcgcggtgggcccgggctcttggtcggccaccctttcttaactgggatccgggcttagttgtcgcaatgtgacaacgggctcgaaagctggggccaggggaccctagtctacgacgcctcgggtgggtgtcccgcacccctccccactttcacggcactcggcgagacctggggagtcaggtgttggggacactttggaggtcaggaacgggagctggggagagggctctgtcagcggggtccagagatgggccgccctccaaggacgccctgcgcggggacaagggcttcttggcctggcctggccgcttcacttgggcgtcagggggggcttcccggggcaggcggtcagtcgaggcgggttggaattctgagtctgggttcggggttcggggttcggccttcatgaacagacagcccaggcgggccgttgtttggcccctgggggcctggttggaatgcgaggtctcgggaagtcaggagggagcctggccagcagagggttcccagccctgcggccgagggacctggagacgggcagggcattggccgtcgcagggccaggccacaccccccaggtttttgtggggcgagcctggagattgcaccactgtgattactatgctatggatctctggggcccaggcgttgaagtcgtcgtgtcctcaggtaagaacggccctccagggcctttaatttctgctctcgtctgtgggcttttctgactctgatcctcgggaggcgtctgtgccccccccggggatgaggccggcttgccaggaggggtcagggaccaggagcctgtgggaagttctgacgggggctgcaggcgggaagggccccaccggggggcgagccccaggccgctgggcggcaggagacccgtgagagtgcgccttgaggagggtgtctgcggaaccacgaacgcccgccgggaagggcttgctgcaatgcggtcttcagacgggaggcgtcttctgccctcaccgtctttcaagcccttgtgggtctgaaagagccatgtcggagagagaagggacaggcctgtcccgacctggccgagagcgggcagccccgggggagagcggggcgatcggcctgggctctgtgaggccaggtccaagggaggacgtgtggtcctcgtgacaggtgcacttgcgaaaccttagaagacggggtatgttggaagcggctcctgatgtttaagaaaagggagactgtaaagtgagcagagtcctcaagtgtgttaaggttttaaaggtcaaagtgttttaaacctttgtgactgcagttagcaagcgtgcggggagtgaatggggtgccagggtggccgagaggcagtacgagggccgtgccgtcctctaattcagggcttagttttgcagaataaagtcggcctgttttctaaaagcattggtggtgctgagctggtggaggaggccgcgggcagccctggccacctgcagcaggtggcaggaagcaggtcggccaagaggctattttaggaagccagaaaacacggtcgatgaatttatagcttctggtttccaggaggtggttgggcatggctttgcgcagcgccacagaaccgaaagtgcccactgagaaaaaacaactcctgcttaatttgcatttttctaaaagaagaaacagaggctgacggaaactggaaagttcctgttttaactactcgaattgagttttcggtcttagcttatcaactgctcacttagattcattttcaaagtaaacgtttaagagccgaggcattcctatcctcttctaaggcgttattcctggaggctcattcaccgccagcacctccgctgcctgcaggcattgctgtcaccgtcaccgtgacggcgcgcacgattttcagttggcccgcttcccctcgtgattaggacagacgcgggcactctggcccagccgtcttggctcagtatctgcaggcgtccgtctcgggacggagctcaggggaagagcgtgactccagttgaacgtgatagtcggtgcgttgagaggagacccagtcgggtgtcgagtcagaaggggcccggggcccgaggccctgggcaggacggcccgtgccctgcatcacgggcccagcgtcctagaggcaggactctggtggagagtgtgagggtgcctggggcccctccggagctggggccgtgcggtgcaggttgggctctcggcgcggtgttggctgtttctgcgggatttggaggaattcttccagtgatgggagtcgccagtgaccgggcaccaggctggtaagagggaggccgccgtcgtggccagagcagctgggagggttcggtaaaaggctcgcccgtttcctttaatgaggacttttcctggagggcatttagtctagtcgggaccgttttcgactcgggaagagggatgcggaggagggcatgtgcccaggagccgaaggcgccgcggggagaagcccagggctctcctgtccccacagaggcgacgccactgccgcagacagacagggcctttccctctgatgacggcaaaggcgcctcggctcttgcggggtgctgggggggagtcgccccgaagccgctcacccagaggcctgaggggtgagactgaccgatgcctcttggccgggcctggggccggaccgagggggactccgtggaggcagggcgatggtggctgcgggagggaaccgaccctgggccgagcccggcttggcgattcccgggcgagggccctcagccgaggcgagtgggtccggcggaaccaccctttctggccagcgccacagggctctcgggactgtccggggcgacgctgggctgcccgtggcaggcctgggctgacctggacttcaccagacagaacagggctttcagggctgagctgagccaggtttagcgaggccaagtggggctgaaccaggctcaactggcctgagctgggttgagctgggctgacctgggctgagctgagctgggctgggctgggctgggctgggctgggctgggctggactggctgagctgagctgggttgagctgagctgagctggcctgggttgagctgggctgggttgagctgagctgggttgagctgggttgagctgggttgatctgagctgagctgggctgagctgagctaggctggggtgagctgggctgagctggtttgagttgggttgagctgagctgagctgggctgtgctggctgagctaggctgagctaggctaggttgagctgggctgggctgagctgagctaggctgggctgatttgggctgagctgagctgagctaggctgcgttgagctggctgggctggattgagctggctgagctggctgagctgggctgagctggcctgggttgagctgagctggactggtttgagctgggtcgatctgggttgagctgtcctgggttgagctgggctgggttgagctgagctgggttgagctgggctcagcagagctgggttgggctgagctgggttgagctgagctgggctgagctggcctgggttgagctgggctgagctgagctgggctgagctggcctgtgttgagctgggctgggttgagctgggctgagctggattgagctgggttgagctgagctgggctgggctgtgctgactgagctgggctgagctaggctggggtgagctgggctgagctgatccgagctaggctgggctggtttgggctgagctgagctgagctaggctggattgatctggctgagctgggttgagctgagctgggctgagctggtctgagctggcctgggtcgagctgagctggactggtttgagctgggtcgatctgggctgagctggcctgggttgagctgggctgggttgagctgagctgggttgagctgggctgagctgagggctggggtgagctgggctgaactagcctagctaggttgggctgagctgggctggtttgggctgagctgagctgagctaggctgcattgagcaggctgagctgggctgagcaggcctggggtgagctgggctaggtggagctgagctgggtcgagctgagttgggctgagctggcctgggttgaggtaggctgagctgagctgagctaggctgggttgagctggctgggctggtttgcgctgggtcaagctgggccgagctggcctgggttgagctgggctcggttgagctgggctgagctgagccgacctaggctgggatgagctgggctgatttgggctgagctgagctgagctaggctgcattgagcaggctgagctgggcctggagcctggcctggggtgagctgggctgagctgcgctgagctaggctgggttgagctggctgggctggtttgcgctgggtcaagctgggccgagctggcctgggatgagctgggccggtttgggctgagctgagctgagctaggctgcattgagcaggctgagctgggctgagctggcctggggtgagctgggctgagctaagctgagctgggctggtttgggctgagctggctgagctgggtcctgctgagctgggctgagctgaccaggggtgagctgggctgagttaggctgggctcagctaggctgggttgatctggcagggctggtttgcgctgggtcaagctcccgggagatggcctgggatgagctgggctggtttgggctgagctgagctgagctgagctaggctgcattgagcaggctgagctgggctgagctggcctggggtgagctgggctgggtggagctgagctgggctgaactgggctaagctggctgagctggatcgagctgagctgggctgagctggcctggggttagctgggctgagctgagctgagctaggctgggttgagctggctgggctggtttgcgctgggtcaagctgggccgagctggcctgggttgagctgggctgggctgagctgagctaggctgggttgagctgggctgggctgagctgagctaggctgcattgagctggctgggatggattgagctggctgagctggctgagctggctgagctgggctgagctggcctgggttgagctgggctgggttgagctgagctgggctgagctgggctcagcagagctgggttgagctgagctgggttgagctggggtgagctgggctgagcagagctgggttgagctgagctgggttgagctgggctcgagcagagctgggttgagctgagctgggttgagctgggctcagcagagctgggttgagctgagctgggttgagctgggctgagctagctgggctcagctaggctgggttgagctgagctgggctgaactgggctgagctgggctgaactgggctgagctgggctgagctgggctgagcagagctgggctgagcagagctgggttggtctgagctgggttgagctgggctgagctgggctgagcagagttgggttgagctgagctgggttcagctgggctgagctaggctgggttgagctgggttgagttgggctgagctgggctgggttgagcggagctgggctgaactgggctgagctgggctgagcggaactgggttgatctgaattgagctgggctgagccgggctgagccgggctgagctgggctaggttgagcttgggtgagcttgcctcagctggtctgagctaggttgggtggagctaggctggattgagctgggctgagctgagctgatctggcctcagctgggctgaggtaggctgaactgggctgtgctgggctgagctgagctgagccagtttgagctgggttgagctgggctgagctgggctgtgttgatctttcctgaactgggctgagctgggctgagctggcctagctggattgaacgggggtaagctgggccaggctggactgggctgagctgagctaggctgagctgagttgaattgggttaagctgggctgagatgggctgagctgggctgagctgggttgagccaggtcggactgggttaccctgggccacactgggctgagctgggcggagctcgattaacctggtcaggctgagtcgggtccagcagacatgcgctggccaggctggcttgacctggacacgttcgatgagctgccttgggatggttcacctcagctgagccaggtggctccagctgggctgagctggtgaccctgggtgacctcggtgaccaggttgtcctgagtccgggccaagccgaggctgcatcagactcgccagacccaaggcctgggccccggctggcaagccaggggcggtgaaggctgggctggcaggactgtcccggaaggaggtgcacgtggagccgcccggaccccgaccggcaggacctggaaagacgcctctcactcccctttctcttctgtcccctctcgggtcctcagagagccagtctgccccgaatctctaccccctcgtctcctgcgtcagccccccgtccgatgagagcctggtggccctgggctgcctggcccgggacttcctgcccagctccgtcaccttctcctggaactacaagaacagcagcaaggtcagcagccagaacatccaggacttcccgtccgtcctgagaggcggcaagtacttggcctcctcccgggtgctcctaccctctgtgagcatcccccaggacccagaggccttcctggtgtgcgaggtccagcaccccagtggcaccaagtccgtgtccatctctgggccaggtgagctgggctccccctgtggctgtggcgggggcggggccgggtgccgccggcacagtgacgccccgttcctgcctgcagtcgtagaggagcagccccccgtcttgaacatcttcgtccccacccgggagtccttctccagtactccccagcgcacgtccaagctcatctgccaggcctcagacttcagccccaagcagatctccatggcctggttccgtgatgggaaacgggtggtgtctggcgtcagcacaggccccgtggagaccctacagtccagtccggtgacctacaggctccacagcatgctgaccgtcacggagtccgagtggctcagccagagcgtcttcacctgccaggtggagcacaaagggctgaactacgagaagaacgcgtcctctctgtgcacctccagtgagtgcagcccctcgggccgggcggcggggcggcgggagccacacacacaccagctgctccctgagccttggcttccgggagtggccaaggcggggaggggctgtgcagggcagctggagggcactgtcagctggggcccagcaccccctcaccccggcagggcccgggctccgaggggccccgcagtcgcaggccctgctcttgggggaagccctacttggccccttcagggcgctgacgctccccccacccacccccgcctagatcccaactctcccatcaccgtcttcgccatcgccccctccttcgctggcatcttcctcaccaagtcggccaagctttcctgcctggtcacgggcctcgtcaccagggagagcctcaacatctcctggacccgccaggacggcgaggttctgaagaccagtatcgtcttctctgagatctacgccaacggcaccttcggcgccaggggcgaagcctccgtctgcgtggaggactgggagtcgggcgacaggttcacgtgcacggtgacccacacggacctgccctcgccgctgaagcagagcgtctccaagcccagaggtaggccctgccctgcccctgcctccgcccggcctgtgccttggccgccggggcgggagccgagcctggccgaggagcgccctcggccccccgcggtcccgacccacacccctcctgctctcctccccagggatcgccaggcacatgccgtccgtgtacgtgctgccgccggccccggaggagctgagcctgcaggagtgggcctcggtcacctgcctggtgaagggcttctccccggcggacgtgttcgtgcagtggctgcagaagggggagcccgtgtccgccgacaagtacgtgaccagcgcgccggtgcccgagcccgagcccaaggcccccgcctcctacttcgtgcagagcgtcctgacggtgagcgccaaggactggagcgacggggagacctacacctgcgtcgtgggccacgaggccctgccccacacggtgaccgagaggaccgtggacaagtccaccggtaaacccaccctgtacaacgtctccctggtcctgtccgacacggccagcacctgctactgaccccctggctgcccgccgcggccggggccagagcccccgggcgaccatcgctctgtgtgggcctgtgtgcaacccgaccctgtcggggtgagcggtcgcatttctgaaaattagaaataaaagatctcgtgccg

SEQ ID NO: 30 - DNA, 12048 bp, Sus scrofa
gcgtccgaagtcaaaaatatctgcagccttcatgtattcatagaaacaaggaatgtctacattttccaaagtgggaccagaatcttgggtcatgtctaaggcatgtgcatttgcacatggtaggcaaaggactttgcttctcccagcacatctttctgcagagatccatggaaacaagactcaactccaaagcagcaaagaagcagcaagttctcaagtgatctcctctgactccctcctcccaggctaatgaagccatgttgcccctgggggattaagggcaggtgtccattgtggcacccagcccgaagacaagcaatttgatcaggttctgagcactcctgaatgtggactctggaattttctcctcaccttgtggcatatcagcttaagtcaagtacaagtgacaaacaacataatcctaagaagagaggaatcaagctgaagtcaaaggatcactgccttggattctactgtgaatgatgacctggaaaatatcctgaacaacagcttcagggtgatcatcagagacaaaagttccagagccaggtagggaaaccctcaagccttgcaaagagcaaaatcatgccattgggttcttaacctgctgagtgatttactatatgttactgtgggaggcaaagcgctcaaatagcctgggtaagtatgtcaaataaaaagcaaaagtggtgtttcttgaaatgttagacctgaggaaggaatattgataacttaccaataattttcagaatgatttatagatgtgcacttagtcagtgtctctccaccccgcacctgacaagcagtttagaatttattctaagaatctaggtttgctgggggctacatgggaatcagcttcagtgaagagtttgttggaatgattcactaaattttctatttccagcataaatccaagaacctctcagactagtttattgacactgcttttcctccataatccatctcatctccgtccatcatggacactttgtagaatgacaggtcctggcagagactcacagatgcttctgaaacatcctttgccttcaaagaatgaacagcacacatactaaggatctcagtgatccacaaattagtttttgccacaatggttcttatgataaaagtctttcattaacagcaaattgttttataatagttgttctgctttataataattgcatgcttcactttcttttcttttctttttttttctttttttgctttttagtgccgcaggtgcagcatatgaaatttcccaggctaggggtcaaatcagaactacacctactggcctacgccacagccacagcaactcaggatctaagccatgtcggtgacctacactacagctcatggcaatgccagatccttaacccaatgagcgaggccagggatcgaacccatgtcctcatggatactagtcaggctcattatccgctgagccataacaggaactcccgagtttgctttttatcaaaattggtacagccttattgtttctgaaaaccacaaaatgaatgtattcacataattttaaaaggttaaataatttatgatatacaagacaatagaaagagaaaacgtcattgcctctttcttccacgacaacacgcctccttaattgatttgaagaaataactactgagcatggtttagtgtacttctttcagcaattagcctgtattcatagccatacatattcaattaaaatgagatcatgatatcacacaatacataccatacagcctatagggatttttacaatcatcttccacatgactacataaaaacctacctaaaaaaaaaaaaaaccctacttcatcctcctattggctgctttgtgctccattaaaaagctctatcataattaggttatgatgaggatttccattttctacctttcaagcaacatttcaatgcacagtcttatatacacatttgagcctacttttctttttctttctttttttggttttttttttttttttttttttggtctttttgtcttttctaaggctgcatatggaggttcccaggctagctgtctaatcagaactatagctgctggcctacgccacatccacagcaatacaagatctgagccatgtctgcaacttacaccacagctcacagcaacggtggatccttaaaccactgagcaaggccagggatcaaacccataacttcatggctcctagttggatttgttaaccactgagccatgatggcaactcctgagcctacttttctaatcatttccaaccctaggacacttttttaagtttcatttttctccccccaccccctgttttctgaagtgtgtttgcttccactgggtgacttcactcccaggatctcatctgcaggatactgcagctaagtgtatgagctctgaatttgaatcccaactctgccactcaaagggataggagtttccgatgtggcccaatgggatcagtggcatctctgcagtgccaggacgcaggttccatccctggcccagcacagtgggttaagaatctggcattgctgcagctgaggcatagatttcaattgtgcctcagatctgatccttggcccaaggactgcatatgcctcagggcaaccaaaaaagagaaaaggggggtgatagcattagtttctagatttgggggataattaaataaagtgatccatgtacaatgtatggcattttgtaaatgctcaacaaatttcaactattatggagttcccatcatggctcagtggaagggaatctgattagcatccatgaggacacaggtccaaccccgaccttgctcagtgggcattgctgtgagctgtggcatgggttacagacgaagctcggatctggcattgctgtggctgtggtgtaagccagcaactacagctctcattcagcccctagcctgggaacctccatatgcctaaaagacaaaaaataaaatttaaattaaaaataaagaaatgttaactattatgattggtactgcttgcattactgcaaagaaagtcactttctatactctttaatatcttagttgactgtgtgctcagtgaactattttggacacttaatttccactctcttctatctccaacttgacaactctctttcctctcttctggtgagatccactgctgactttgctctttaaggcaactagaaaagtgctcagtgacaaaatcaaagaaagttaccttaatcttcagaattacaatcttaagttctcttgtaaagcttactatttcagtggttagtattattccttggtcccttacaacttatcagctctgatctattgctgattttcaactatttattgttggagttttttccttttttccctgttcattctgcaaatgtttgctgagcatttgtcaagtgaagatactggactgggccttccaaatataagacaatgaaacatcgggagttctcattatggtgcagcagaaacgaatccaactaggaaatgtgaggttgcaggttcgatccctgcccttgctcagtgggttaaggatccagcattaccgtgagctgtggtgtaggttgcagacgtggctcagatcctgcgttgctgtggctgtggcataggctggcagctctagctctgattcgaccgctagcctgggaacctccatgcgccccgagtgcagcccttaaaaagcaaaaaaaaaagaaagaaagaaaaagacaatgaaacatcaaacagctaacaatccagtagggtagaaagaatctggcaacagataagagcgattaaatgttctaggtccagtgaccttgcctctgtgctctacacagtcgtgccacttgctgagggagaaggtctctcttgagttgagtcctgaaagacattagttgttcacaaactaatgccagtgagtgaaggtgtttccaagcagagggagagtttggtaaaaagctggaagtcacagaaagactctaaagagtttaggatggtgggagcaacatacgctgagatggggctggaaggttaagagggaaacaactatagtaagtgaagctggactcacagcaaagtgaggacctcagcatccttgatggggttaccatggaaacaccaaggcacaccttgatttccaaaacagcaggcacctgattcagcccaatgtgacatggtgggtacccctctagctctacctgttctgtgacaactgacaaccaacgaagttaagtctggattttctactctgctgatccttgtttttgtttcacacgtcatctatagcttcatgccaaaatagagttcaaggtaagacgcgggccttggtttgatatacatgtagtctatcttgtttgagacaatatggtggcaaggaagaggttcaaacaggaaaatactctctaattatgattaactgagaaaagctaaagagtcccataatgacactgaatgaagttcatcatttgcaaaagccttcccccccccccaggagactataaaaaagtgcaattttttaaatgaacttatttacaaaacagaaatagactcacagacataggaaacgaacagatggttaccaagggtgaaagggagtaggagggataaataaggagtctggggttagcagatacaccccagtgtacacaaaataaacaacagggacctactatatagcacagggaactatatgcagtagcttacaataacctataatggaaaagaatgtgaaaaagaatatatgtatgcgtgtgtgtgtaactgaatcactttgctgtaacctgaatctaacataacattgtaaatcaactacagttttttttttttttaagtgcagggttttggtgttttttttttttcatttttgtttttgtttttgttttttgctttttagggccacacccagacatatgggggttcccaggctaggggtctaattagagctacagttgccggcttgcaccacagccacagcaacatcagatccgagccgcacttgcgacttacaccacagctcatggcaataccagatccttaacccactgagcaaggcccagggatcgtacccgcaacctcatggttcctagtcagattcatttctgctgcgctacaatgggaactccaagtgcagttttttgtaatgtgcttgtctttctttgtaattcatattcatcctacttcccaataaataaataaatacataaataataaacataccattgtaaatcaactacaattttttttaaatgcagggtttttgttttttgttttttgttttgtctttttgccttttctagggccgctcccatggcatatggaggttcccaggctaggggtcgaatcggagctgtagccaccggcctacgccagagccacagcaacgcgggatccgagccgcgtctgcaacctacaccacagctcacggcaacgccggatcgttaacccactgagcaagggcagggatcgaacctgcaacctcatggttcctagtcagattcgttaactactgagccacaacggaaactcctaaagtgcagtttttaaatgtgcttgtctttctttgtaatttacactcaacctacttcccaataaataaataaataaacaaataaatcatagacatggttgaattctaaaggaagggaccatcaggccttagacagaaatacgtcatcttctagtattttaaaacacactaaagaagacaaacatgctctgccagagaagcccagggcctccacagctgcttgcaaagggagttaggcttcagtagctgacccaaggctctgttcctcttcagggaaaagggtttttgttcagtgagacagcagacagctgtcactgtggtggacgttcggccaaggaaccaagctggaactcaaacgtaagtcaatccaaacgttccttccttggctgtctgtgtcttacggtctctgtggctctgaaatgattcatgtgctgactctctgaaaccagactgacattctccagggcaaaactaaagcctgtcatcaaactggaaaactgagggcacattttctgggcagaactaagagtcaggcactgggtgaggaaaaacttgttagaatgatagtttcagaaacttactgggaagcaaagcccatgttctgaacagagctctgctcaagggtcaggaggggaaccagtttttgtacaggagggaagttgagacgaacccctgtgtatatggtttcggcgcggggaccaagctggagctcaaacgtaagtggctttttccgactgattctttgctgtttctaattgttggttggctttttgtccatttttcagtgttttcatcgaattagttgtcagggaccaaacaaattgccttcccagattaggtaccagggaggggacattgctgcatgggagaccagagggtggctaatttttaacgtttccaagccaaaataactggggaagggggcttgctgtcctgtgagggtaggtttttatagaagtggaagttaaggggaaatcgctatggttcacttttggctcggggaccaaagtggagcccaaaattgagtacattttccatcaattatttgtgagatttttgtcctgttgtgtcatttgtgcaagtttttgacattttggttgaatgagccattcccagggacccaaaaggatgagaccgaaaagtagaaaagagccaacttttaagctgagcagacagaccgaattgttgagtttgtgaggagagtagggtttgtagggagaaaggggaacagatcgctggctttttctctgaattagcctttctcatgggactggcttcagagggggtttttgatgagggaagtgttctagagccttaactgtgggttgtgttcggtagcgggaccaagctggaaatcaaacgtaagtgcacttttctactcctttttctttcttatacgggtgtgaaattggggacttttcatgtttggagtatgagttgaggtcagttctgaagagagtgggactcatccaaaaatctgaggagtaagggtcagaacagagttgtctcatggaagaacaaagacctagttagttgatgaggcagctaaatgagtcagttgacttgggatccaaatggccagacttcgtctgtaaccaacaatctaatgagatgtagcagcaaaaagagatttccattgaggggaaagtaaaattgttaatattgtggatcacctttggtgaagggacatccgtggagattgaacgtaagtattttttctctactaccttctgaaatttgtctaaatgccagtgttgacttttagaggcttaagtgtcagttttgtgaaaaatgggtaaacaagagcatttcatatttattatcagtttcaaaagttaaactcagctccaaaaatgaatttgtagacaaaaagattaatttaagccaaattgaatgattcaaaggaaaaaaaaattagtgtagatgaaaaaggaattcttacagctccaaagagcaaaagcgaattaattttctttgaactttgccaaatcttgtaaatgatttttgttctttacaatttaaaaaggttagagaaatgtatttcttagtctgttttctctcttctgtctgataaattattatatgagataaaaatgaaaattaataggatgtgctaaaaaatcagtaagaagttagaaaaatatatgtttatgttaaagttgccacttaattgagaatcagaagcaatgttatttttaaagtctaaaatgagagataaactgtcaatacttaaattctgcagagattctatatcttgacagatatctcctttttcaaaaatccaatttctatggtagactaaatttgaaatgatcttcctcataatggagggaaaagatggactgaccccaaaagctcagatttaaagaaatctgtttaagtgaaagaaaataaaagaactgcattttttaaaggcccatgaatttgtagaaaaataggaaatattttaataagtgtattcttttattttcctgttattacttgatggtgtttttataccgccaaggaggccgtggcaccgtcagtgtgatctgtagaccccatggcggccttttttcgcgattgaatgaccttggcggtgggtccccagggctctggtggcagcgcaccagccgctaaaagccgctaaaaactgccgctaaaggccacagcaaccccgcgaccgcccgttcaactgtgctgacacagtgatacagataatgtcgctaacagaggagaatagaaatatgacgggcacacgctaatgtggggaaaagagggagaagcctgatttttattttttagagattctagagataaaattcccagtattatatccttttaataaaaaatttctattaggagattataaagaatttaaagctatttttttaagtggggtgtaattctttcagtagtctcttgtcaaatggatttaagtaatagaggcttaatccaaatgagagaaatagacgcataaccctttcaaggcaaaagctacaagagcaaaaattgaacacagcagccagccatctagccactcagattttgatcagttttactgagtttgaagtaaatatcatgaaggtataattgctgataaaaaaataagatacaggtgtgacacatctttaagtttcagaaatttaatggcttcagtaggattatatttcacgtatacaaagtatctaagcagataaaaatgccattaatggaaacttaatagaaatatatttttaaattccttcattctgtgacagaaattttctaatctgggtcttttaatcacctaccctttgaaagagtttagtaatttgctatttgccatcgctgtttactccagctaatttcaaaagtgatacttgagaaagattatttttggtttgcaaccacctggcaggactattttagggccattttaaaactcttttcaaactaagtattttaaactgttctaaaccatttagggccttttaaaaatcttttcatgaatttcaaacttcgttaaaagttattaaggtgtctggcaagaacttccttatcaaatatgctaatagtttaatctgttaatgcaggatataaaattaaagtgatcaaggcttgacccaaacaggagtatcttcatagcatatttcccctcctttttttctagaattcatatgattttgctgccaaggctattttatataatctctggaaaaaaaatagtaatgaaggttaaaagagaagaaaatatcagaacattaagaattcggtattttactaactgcttggttaacatgaaggtttttattttattaaggtttctatctttataaaaatctgttcccttttctgctgatttctccaagcaaaagattcttgatttgttttttaactcttactctcccacccaagggcctgaatgcccacaaaggggacttccaggaggccatctggcagctgctcaccgtcagaagtgaagccagccagttcctcctgggcaggtggccaaaattacagttgacccctcctggtctggctgaaccttgccccatatggtgacagccatctggccagggcccaggtctccctctgaagcctttgggaggagagggagagtggctggcccgatcacagatgcggaaggggctgactcctcaaccggggtgcagactctgcagggtgggtctgggcccaacacacccaaagcacgcccaggaaggaaaggcagcttggtatcactgcccagagctaggagaggcaccgggaaaatgatctgtccaagacccgttcttgcttctaaactccgagggggtcagatgaagtggttttgtttcttggcctgaagcatcgtgttccctgcaagaagcggggaacacagaggaaggagagaaaagatgaactgaacaaagcatgcaaggcaaaaaaggccttaggatggctgcaggaagttagttcttctgcattggctccttactggctcgtcgatcgcccacaaacaacgcacccagtggagaacttccctgttacttaaacaccattctctgtgcttgcttcctcaggggctgatgccaagccatccgtcttcatcttcccgccatcgaaggagcagttagcgaccccaactgtctctgtggtgtgcttgatcaataacttcttccccagagaaatcagtgtcaagtggaaagtggatggggtggtccaaagcagtggtcatccggatagtgtcacagagcaggacagcaaggacagcacctacagcctcagcagcaccctctcgctgcccacgtcacagtacctaagtcataatttatattcctgtgaggtcacccacaagaccctggcctcccctctggtcacaagcttcaacaggaacgagtgtgaggcttagaggcccacaggcccctggcctgcccccagccccagcccccctccccacctcaagcctcaggcccttgccccagaggatccttggcaatcccccagcccctcttccctcctcatcccctccccctctttggctttaaccgtgttaatactggggggtgggggaatgaataaataaagtgaacctttgcacctgtgatttctctctcctgtctgattttaaggttgttaaatgttgttttccccattatagttaatcttttaaggaactacatactgagttgctaaaaactacaccatcacttataaaattcacgccttctcagttctcccctcccctcctgtcctccgtaagacaggcctccgtgaaacccataagcacttctctttacaccctctcctgggccggggtaggagactttttgatgtcccctcttcagcaagcctcagaaccattttgagggggacagttcttacagtcacattcctgtgatctaatgactttagttaccgaaaagccagtctctcaaaaagaagggaacggctagaaaccaagtcatagaaatatatatgtataaaatatatatatatccatatatgtaaaataacaaaataatgataacagcataggtcaacaggcaacagggaatgttgaagtccattctggcacttcaatttaagggaataggatgccttcattacattttaaatacaatacacatggagagcttcctatctgccaaagaccatcctgaatgccttccacactcactacaaggttaaaagcattcattacaatgttgatcgaggagttcccgttgtggctcagcaggttaagaacgtgactggtatccaggaggatgcgggtttggtccccagcctcgctcagtggattaaggatccagtgttgctgcaagatcacgggctcagatcccgtgttctatggctatggtgtaggctggtagctgcatgcagccctaatttgacccctagcctgggaactgccatatgccacatgtgaggcccttaaaacctaaaagaaaaaaaaagaaaagaaatatcttacacccaatttatagataagagagaagctaaggtggcaggcccaggatcaaagccctacctgcctatcttgacacctgatacaaattctgtcttctagggtttccaacactgcatagaacagagggtcaaacatgctaccctcccagggactcctcccttcaaatgacataaattttgttgcccatctctgggggcaaaactcaacaatcaatggcatctctagtaccaagcaaggctcttctcatgaagcaaaactctgaagccagatccatcatgacccaaggaagtaaagacaggtgttactggttgaactgtatccttcaattcaatatgctcaatttccaactcccagtccccgtaaatacaaccccctttgggaagagagtccttgcagatgtagccacgttaaaaagagattatacagaaaggctagtgaggatgcagtgaaacgggatctttcatacattgctggtggaaatgtaaaatgctgcaggcactctagaaaataatttgccagttttttgaaaagctaaacaaaatagtttagttgcattctgggttatttatcccccagaaattaaaaattatgtccgcacaaaaacgtgtacataatcattcataacagccttgtac

SEQ ID NO: 31 - DNA, 126068 bp, Bos sp.
  misc_feature (3591)..(3690): n is a, c, g, or t
tgggttctatgccacccagcttggtctctgatggtcacttgaggcccccatctcatggcaaagagggaactggattgcagatgagggaccgtgggcagacatcagagggacacagaaccctcaaggctggggaccagagtcagagggccaggaagggctggggaccttgggtctagggatccgggtcagggactcggcaaaggtggagggctccccaaggcctccatggggcggacctgcagatcctgggccggccagggacccagggaaagtgcaaggggaagacgggggaggagaaggtgctgaactcagaactggggaaagagataggaggtcaggatgcaggggacacggactcctgagtctgcaggacacactcctcagaagcaggagtccctgaagaagcagagagacaggtaccagggcaggaaacctccagacccaagaagactcagagaggaacctgagctcagatctgcggatggggggaccgaggacaggcagacaggctccccctcgaccagcacagaggctccaagggacacagacttggagaccaacggacgccttcgggcaaaggctcgaacacacatgtcagctcaaaatatacctggactgactcacaggaggccagggaggccacatcatccactcaggggacagactgccagccccaggcagaccccatcaaccgtcagacgggcaggcaaggagagtgagggtcagatgtctgtgtgggaaaccaagaaccagggagtctcaggacagcgctggcaggggtccaggctcaggctttcccaggaagatggggaggtgcctgagaaaaccccacccaccttccctggcacaggccctctggctcacagtggtgcctggactcggggtcctgctgggctctcaaaggatcctgtgtccccctgtgacacagactcaggggctcccatgacgggcaccagacctctgattgtggtcttcttcccctcgcccactttgcaggtcagcccaagtccacaccctcggtcaccctgttcccgccctccaaggaggagctcagcaccaacaaggccaccctggtgtgtctcatcagcgacttctacccgggtagcgtgaccgtggtctagaaggcagacggcagcaccatcacccgcaacgtggagaccacccgggcctccaaacagagcaacagcaagtacgcggccagcagctacctgagcctgatgggcagcgactggaaatcgaaaggcagttacagctgcgaggtcacgcacgaggggagcaccgtgacgaagacagtgaagcctcagagtgttcttagggccctgggcccccaccccggaaagttctaccctcccaccctggttccccctagcccttcctcctgcacacaatcagctcttaataaaatgtcctcattgtcattcagaaatgaatgctctctgctcatttttgttgatacatttggtgccctgagctcagttatcttcaaaggaaacaaatcctcttagcctttgggaatcaggagagagggtggaagcttgggggtttggggagggatgatttcactgtcatccagaatcccccagagaacattctggaacaggggatggggccactgcaggagtggaagtctgtccaccctccccatcagccgccatgcttcctcctctgtgtggaccgtgtccagctctgatggtcacggcaacacactctggttgccacgggcccagggcagtatctcggctccctccactgggtgctcagcaatcacatctggaagctgctcctgctcaagcggccctctgtccacttagatgatgacccccctgaagtcatgcgtgttttggctgaaaccccaccctggtgattcccagtcgtcacagccaagactccccccgactcgacctttccaagggcactaccctctgcccctcccccagggctccccctcacagtcttcaggggaccggcaagcccccaaccctggtcactcatctcacagttcccccaggtcgccctcctcccacttgcatggcaggagggtcccagctgacttcgaggtctctgaccagcccagctctgctctgcgaccccttaaaactcagcccaccacggagcccagcaccatctcaggtccaagtggccgttttggttgatgggttccgtgagctcaagcccagaatcaggttagggaggtcgtggcgtggtcatctctgaccttgggtggtttcttaggagctcagaatgggagctgatacacggataggctgtgctaggcactcccacgggaccacacgtgagcaccgttagacacacacacacacacacacacacacacacacacacacacgagtcactacaaacacggccatgttggttggacgcatctctaggaccagaggcgcttccagaatccgccatggcctcactctgcggagaccacagctccatcccctccgggctgaaaaccgtctcctcaccctcccaccggggtgacccccaaagctgctcacgaggagcccccacctcctccaggagaagttccctgggacccggtgtgacacccagccgtccctcctgcccctcccccgcctggagatggccggcgccccatttcccaggggtgaactcacaggacgggaggggtcgctcccctcacccgcccggagggtcaaccagcccctttgaccaggaggggggcggacctggggctccgagtgcagctgcaggcgggcccccgggggtggcggggctggcggcagggtttatgctggaggctgtgtcactgtgcgtgtttgctcggtggagggacccagctggccatccggggtgagtctcccctttccagctttccggagtcaggagtgacaaatgggtagattcttgtgtttttcttacccatctggggctgaggtctccgtcaccctaggcctgtaaccctcccccttttagcctgttccctctgggcttcttcacgtttccttgagggacagtttcactgtcacccagcaaagcccagagaatatccagatggggcaggcaatatgggacggcaagctagtccaccctcttaccttgggctccccgcggcctccggataatgtctgagctgcctccctggatgcttcaccttctgagactgtgaggcaagaaaccccctccccaaaagggaggagacccgaccccagtgcagatgaacgtgctgtgaggggaccctgggagtaagtggggtctggcggggaccgtgatcattgcagactgatgccccaggcagggtgagaggtcatggccgccgacaccagcagctgcagggagcacaggccgggggcaagtcatgcagacaggacaggacgtgtgaccctgaagagtcagagtgacacgcggggggggggcccggagctcccgagattagggcttgggtcctaacgggatccaggagggtccacgggcccaccccagccctctccctgcacccaatcaacttgcaataaaacgtcctctattgtcttacaaaaaccctgctctctgctcatgtttttccttgccccgcatttaatcgtcaacctctccaggattctggaactggggtggggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcttatgtggtgggcaggggggtagtaagatcaaaagtgcttaaattaataaagccggcatgatatacgagtttggataaaaaatagatggaaaagtaagaaaggacaggaggggggtgaggcggaagaaagggggaagaaggaaaaaaaaataagagagaggaacaaagaaagggaggggggccggtgatgggggtgggatagaatataataattggagtaaagagtagcgggtggctgttaattccgggggggaatagagaaaaaaaaaaaaaaatgtgcgggtgggcggtaagtatggagattttataaatattatgtgtggaataatgagcgggggtggacgggcaaggcgagagtaaaaaggggcgagagaaaaaaattaggatggaatatatggggtaaattttaaatagagggtgatatatgttagattgagcaagatataaatatagatggtgggggaaaagagacaagggtgagcgccaaaacgccctcccgtatcatttgccttccttcctttaccacctcgttcaaactctttttcgagaaccctgaagcggtcaggcccggggctgggggtgggatacccggggaggggctgcgcctcctcctttgcagagggggtcgaggagtgggagctgaggcaggagactggcaggctggagagatggctgttgacttcctgcctgtttgaactcacagtcacagtgccagacccactgaattgggctaaataccatatttttctggggagagagtgtagagcgagcgactgaggcgagctcatgtcatctacagggccgccagctgcagggactttgtgtgtgtcgtgctcgttgctcagttgtgtccgactctttatgacttcatggactgtaacctgccaggctcctctgtccgtggaattctccaggcaagaatactggagtgggtagccattctcatctccgggggatcttcctgacccaagaatcaaacctgagtctcccgcattgcaggcagcttctttcttgtctgagccaccagggaagccccttaagtggaggatctaaatagagtgtttaggagtataagagaaaggaaggacgtctatacaagatccttcggttcctgtaactacgactcgagttaacaagccctgtgtgagtgagttgccagtaattattgctaacctgtttctttcactcactgagccaggtatcctgtgagacggcatacttacctcctcttctgcattcctcgggatggagctgtgcggtggcctctaggactaccacatcgaccaggtcagacccagggacagaggattgctgagatgcactgagaagtttgtcagcctaggtcttcacccacacagactgtgctgtcgtctaccacgtaattcttcctgtccaaagaactggttaaacgctcctgaagcgtattctggtctgcttcaaaaagtgcctctttcctttataagttccgccaatcctggactttgtcccaggccagtctactttatttgtgggaaaggtttttttggtcttttttgttttaaactctgcagaaattgcttacacttttggtgtgcaatggctcactcttacggttctagctgtattcaaaggggttgcttttctttgtttttaaagctttttgaacgtggaccatttttaaagtctttattaaacgtctaacatcgtttctggtttattttctggtggtctggccatgaggcctacgggtcttagctcccctaccagggtccaacccacatcccttgcactggacggcaaggtcttaacctttgaaccaccagagagcttctgaaaggggctgcttttctccaatcctctttgctccctgcctgctggtagggattcagcacccctgcaatagccctgtctgttcttaggggctcagtagcctttctgcctgggtgtggagctggggttgtaagagagcttcatggatttggacacgacctacgactcagaggtaagactccatcttagcgctgtaatgacctctttccaacaaccacccccaccaccctggaccactgatcaggagagatgattctctctcttatcatcaacgtggtcagtcccaaacttgcacccggcctgtcatagatgtagcaggtaagcaataaatatttgttgaatgttaagtgaattgaaataacataagtgaaaaagaaaacacttaaaaacatgtgtttttataattacacagtaaacatataatcattgtagaaaaaaatcgaaagagtggcgggggccaagtgaaaaccaccatccctggtatgtccacccgcccgggtagccccaggtaagaggtgcggacacggatggccctgtagacacagagacacacgctcatatgctgggtcttgtcttgtgacctcttggggatgatgttattttcacgatgccattcaaaccttctaccacaccatttttagagggtcgttcatcgtaaatcagttcactgctttgttttctgattttgaaagtgtcacattcttcgagaaatgagaaggaacaggcgcgcataaggaagaaagtaaacacgtggccttgcttccagggggcactcagcgtgttggtgtgcacgctggcagtcttttctctgtgacagtcatggccttttcccaaaggtgggctcagataagaccgcctcccatcccctgtccctgtccccgtcccctacggtggaacccacccacggcacgtctccgaggccctttggggctgtggacgttaggctgtgtggacatgctgctggtggggacccagggctgggcagcacgttgtccctgggtcccgggccagtgaggagctcccaaggagcagggctgctgggccaaagggcagtgcgtcccgaggccatggacaaggggatacatttcctgctgaagggctggactgcgtctccctggggccccttggagtcatgggcagtggggaggcctctgctcaccccgttgcccacccatggctcagtctgcagccaggagcgcctggggctgggacgccgaggccggagcccctccctgctgtgctgacgggctcggtgaccctgccgccccctccctggggccctgctgaccgcgggggccaccccggccagttctgagattcccctggggtccagccctccaggatcccaggacccaggatggcaaggatgttgaggaggcagctagggggcagcatcaggcccagaccggggctgggcaggggctgggcgcaggcgggtgggggggtctgcacncccccacctgcnagctgcncnnncntttgntnncgtcctccctgntcctggtctgtcccgcccggggggccccccctggtcttgtttgttccccctccccgtcccttcccccctttttccgtcctcctcccttcttttattcgccccttgtggtcgttttttttccgtccctcttttgtttttttgtctttttctttttccccctcttctcccttgctctctttttcattcgtcggtttttctgctcccttccctctcccccccgctttttttccctgtctgctttttgtgttctccctctctaccccccctgcagcctattttttttatatatccatttccccctagtatttggcccccgcttacttctccctaatttttattttcctttctttaactaaaatcaccgtgtggttataagttttaaccttttttgcaccgcccacaatgcaatcttcacgcacgccccccccgtcagcctccttaaatacctttgcctactgcccccctccttgtataataacgcgtcacgtggtcaaccattatcacctctccaccaccttaccacattttccttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaaaaaagaaaaggctgggcaggttttaatatgggggggttggagtggaatgaaaatgcattggagtggttgcaacaaatggaaaggtctcaggagcgctcctcccccatcaggagctggaaagaagtggaagcaaagcaaggaattcgtgtgatggccagaggtcaggggcagggagctgcaaagactgccggctgtttgtgactgnccgtctccgggtgcatttgttagcagggaggcattacactcatgtcttggtttgctaactaattcttactattgtttagttgcaaggtcatgtctgactctttgcaacccagggactgcagcccgccaggctcctctgtccatgggatttcgcaggcaagaatactggaggtggtagccattttcttcaccatgggatcttcccgagccagaaatggaacccgagtcgcctcctgtgcatggggtctgctgcctaacaggcagatatttgacgtctgagccaacagggaggacagacggtaattataccaaccattgaaagaggaattacacactaatctttatcaaaatctttcaaacagtagaggagaaaggatactctctagtttattccataaagttggaattacgcttatcaataaagacattacaagaaaagaaagtgaagccccaaatgccttataaatatacaagaaaaaatcttttaagatattagccaacttaatcaacaaaaaatgtatcaaaagtccaagtaacattcaccccaggaatgcaagtgtggttcagcctaagacaatcagtcatgagtataccacggaaacaaattaaagagaaaagacattaaatctcacaaatggtgcagaaaaagatttggcaatatcgaacatcttttcatgaccaaaggaaaaaaaagaaacaaaacaccagaaaattctgtgtagaaagaatatatctcaacccaatgaagggcatttatgaaaaacccacagcatacatcacactccatgagaaagactgaaagctttccccactgccattgaactctgtcctggaaattctagtcacagcgacagaacaagagaaagaaataacggccgtctaaactggtaggaagaaatcaaagcgtctctattctctgggcgcataatacaatatagacaaatttctaaagtccacaaaaattcctagagctcataatgaatccagaaatgcgtcagggctcaagattcagatgcaaaaatcgtctgggttttgatgcaccaacaaacaattccattaacaataataccaaggaattaatttaacttagaagagaaaagacctgtttacagagagttataaaacatttggtgatgaaattaaataagagtaaatcatatagaaacaccgttcgtgttttggagacctaatgtcataaacgtggcaacacagagacgcctcacggggaaccctgagcctccttctccaaacaggcctgctcatcatttcacaggtaacctgagaccctaaagcttgactctgaggcactttgagggcatgaagagagcagtagctcctcccatgggaccgacagtcaaggcccagggaatgaccacctggacagatgacttcccggcctcatcagcagtcggtgcagagtggccaccagggggcagcagagagtcgctcaacactgcacctggagatgaggcaacctgggcatcaggtgcccatgcaggggctggatacccacacctcacacctgaggacaggggccggctttctgtggtgtcgccctctcaggatgcacagactccaccctcttcgcttgcattgacagcctctgtccttcctggaggacaagctccaccttccccatctctccccagggggctggggccaacagtgttctctcttgtccactccaggaacacagagccaagagatttatttgtcttaattagaaaaactatttgtattcctgcatttccccagtaactgaaggcaactttaaaaaatgtatttcctggacttccctggtgggccagtggctagactctgagctcccagtgcatggggcctgggttcaatccctgctcaggaaactacatcccacaggctgcaaataagatcctgcatgccacccgatgcaggcaaagaaacaagtgttcggtatgcatgtatttcacgtgaggtgtttctataatttacagccagtattctgtcttacacttagtcattcctttgagcacatgatcggtcgatggcccagaccacacacaggaatactgaggcccagcacccaccggctgcccagaacctcatggccaagggtggacacttacaggacctcaggggacctttaagaacgccccgtgctcttggcagcggagcagtgttaagcatggctctgtccctcgggagctgtgtctgggctgcgtgcatcacctgtggtgtgggcctggtgagggtcaccgtccaggggccctcgagggtcagaagaaccttcccttaaaagttctagaggtggagctagaaccagacccacatgtgaactgcacccaaaaacagtgaaggatgagacacttcaaagtcctgggtgaaattaagggccttcccctgaaccaggatggagcagaggaaggacttggcttccaggaaaccctgacgtctccaccgtgactctggccggggtcatggcagggcccaggatcctttggtgcaaaggactcagggttcctggaaaatacagtctccacctctgagccctcagtgagaagggcttctctcccaggagtggggcaaggacccagattggggtggagctgtccccccagaccctgagaccagcaggtgcaggagcagccccgggctgaggggagtgtgagggacgttccccccgctctcaaccgctgtagccctgggctgagcctctccgaccacggctgcaggcagcccccaccccaccccccgaccctggctcggactgatttgtatccccagcagcaaggggataagacaggcctgggaggagccctgcccagcctgggtttggcgagcagactcagggcgcctccaccatggcctggaccccctcctcctcggcctcctggctcactgcacaggtgagccccagggtccacccaccccagcccagaactcggggacaggcctggccctgactctgagctcagtgggatctgcccgtgagggcaggaggctcctggggctgctgcagggtgggcagctggaggggctgaaatccccctctgtgctcactgctaggtcagccctgagggctgtgcctgccagggaaaggggggtctcctttactcagagactccatccaccaggcacatgagccgggggtgctgagactgacggggagggtgtccctgggggccagagaatctttggcacttaatctgcatcaggcagggggcttctgttcctaggttcttcacgtccagctacctctcctttcctctcctgcaggcgctgtgtcctcctacgagctgactcagtcacccccggcatcgatgtccccaggacagacggccaggatcacgtgttgggggcccagcgttggaggtganaatgttgagtggcaccagcagaagccaggccaggcctgtgcgctggtctcctatggtgacgataaccgacccacgggggtccctgaccagttctctggcgccaactcagggaacatggccaccctgcccatcagcggggcccgggccaaggatgaggccgactattactgtcagctgtgggacagcagcagtaacaatcctcacagtgacacaggcagacgggaagggagatgcaaaccccctgcctggcccgcgcggcccagcctcctcggagcagctgcaggtcccgctgaggcccggtgccctctgtgctcagggcctctgttcatcttgctgagcagcggcaagtgggcattggttccaagtcctgggggcatatcagcacccttgagccagagggttaggggttagggttagggttaggctgtcctgagtcctaggacagccgtgtcccctgtccatgctcagcttctctcaggactggtgggaagattccagaaccaggcaggaaaccgtcagtcgcttgtggccgctgagtcaggcagccattctggtcagcctaccggatcgtccagcactgagacccggggcctccctggagggcaggaggtgggactgcagcccggcccccacaccgtcaccccaaaccctcggagaaccgcgctccccaggacgcctgcccctttgcaacctgacatccgaacattttcatcagaacttctgcaaaatattcacaccgctcctttatgcacattcctcagaagctaaaagttatcatggcttgctaaccactctccttaaatattcttctctaacgtccatcttccctgctccttagacgcgttttcattccacatgtcttactgcctttggtctgctcgtgtattttctttttttttttttttttattggaatatatttgcgttacaatgttgaatttgaattggtttctgttgtacaacaatgtgaattagttatacatgtcctgaggaggggcggctgcgtgggtgcaggagggccgagaggagctactccacgttcaaggtcaggaggggcggccgtgaggagatacccctcgtccaaggtaagagaaacccaagtaagacggtaggtgttgcgagagggcatcagagggcagacacactgaaaccataatcacagaaactagccaatgtgatcacacggaccacagcctggtctaactcagtgaaactaagccatgcccatggggccaaccaagatgggcgggtcatgtgcccatggggccaaccaagatgggcgggtcatggtgaagaggtctgatggaatgtggtccactggagaagggaaaggcaaaccacttcagtattcttgccttgagagccccatgaacagtatgaaaaggcaaaatgataggatactgaaagaggaactccccaggtcagtaggtgcccaatatgctactggagatcagtggagaaataactccagaaagaatgaagggatggagccaaagcaaaaacaatacccagttgtggatgtgactggtgatagaagcaagggccaatgatgtaaagagcaatattgcataggaacctggaatgttaagtccaagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaattttgagcattactttactagcgtgtgagacgagtgcaattgtgcggtagtttgagcattctttggcattgcctttctttgggattggaatgaaaactgacctgttccaggcctgtggccactgctgagttttccaaatttgctggcgtattgagtgcatcactttaacagcatcatcttttaggatttgaaatagctcaactggaattctatcactttagctaattccattcattagctttgtttgtagtgatgcttcctaaggcccccctggctttatcttcctggatgtctggctctggtgagtgatcacaccgctgtgattatctgggtcatgaaggtctttttgtatagttcttcttaggaacagatattatgatctccatccttgcatctcgttatatctagagaagcactgactcccttcatggtgacgtcagatcctcatgactaacaaatggccttttgtaagatgagtgcctcatggtattgagctcccccgtcaccaagaccttatgactgacctcccccactgccccaggtgcctctcgaagcgtctgagatgccgcctcccaggctgcactcctcattttgcccccaataaaacttaacttgcagctctccagctgtgcatctgtgtttagttgacagtacaaatataatggaaaatttaaattaaatataatctatggggagaaatccaaacatcttatgagggagagagagggagagaaaggaaagaagaagaagcaggaggaggaggagagtagagaaacagggggagggcggcagggagacagaggggaggacaccgaggggaaagggaggaaggcgagtgcagtgagagagaggccagagttcatcagagtctggactcgcagcccaatcccacgggtgtgtcccgaagcaggggagagcctgagccaggcggagacagagctgtgtctccagtcctcgtggccgtgacctggagctgtgtggtcagcccccctgaccccagcctggccctgctggtggtcggaggcagtgatcctggacacagtgtctgagcgtctgtctgaaatccctgtggaggcgccactcaggacggacctcgcctggccccacctggatctgcaggtccaggcccgagtggggcttcctgcctggaactgagcagctggaggggcgtctgcaccccagcagtggagcggccccaggggcgctcagagctgccggggggacacagagcttgtctgagacccagggctcgtctccgaggggtcccctaaggtgtcttctggccagggtcagagccgggatgagcacaggtctgagtcagactttcagagctggtggctgcatccctggggacagagggctgggtcctaacctgggggtcagagggcaggacgggagcccagctgacccctggggactggcctcctctgtggtctcccctgggcagtcacagcttccccggacgtggactctgaggaggacagctggggcctggctgtcaggagggggttcgagaggccacactcagaggaggagaccctggcctgcttgggttgtgactgagtttttggggtcctctaggagactctggccctgcaggccctgcaaggtcatctctagtggagcaggactccacaagattgatgaactgaatcctctaggagaggtgtggttgtgagggggcagcattctagaaccaacagcgtgtgcaggtagctggcaccgggtctagtggcggcgggcagggcactcagggccgactaggggtctgggggattcaatggtgcccacagcactgggtcttccatcagaatcccagacttcacaaggcagtttcggggattaggtcaggacgtgagggccacagagaggtggtgatggcctagacaagtccttcacagagagagctccaggggccatgataagatggatgggtctgtattgtcagtttccccacatcaacaccgtggtcccgccagcccataatgctctgtggatgcccctgtgcagagcctacctggaggcccgggaggcggggccgcctgggggctcagctccggggtaaccgggccaggcctgtccctgctgtgtccacagtcctcccggggttggaggagagtgtgagcaggacaggagggtttgtgtctcacttccctggctgtctgtgtcactgggaacattgtaactgccactggcccacgacagacagtaatagtcggcttcatcctcggcacggaccccactgatggtcaagatggctgttttgccggagctggagccagagaactggtcagggatccctgagcgccgcttactgtctttataaatgaccagcttaggggcctggcccggcttctgctggtaccactgagtatattgttcatccagcagctcccccgagcaggtgatcttggccgtctgtcccaaggccactgacactgaagtcaactgtgtcagttcataggagaccacggagcctggaagagaggagggagaggggatgagaaggaaggactccttccccaagtgagaagggcgcctcccctgaggttgtgtctgggctgagctctgggtttgaggcaggctcagtcctgagtgctgggggaccagggccggggtgcagtgctggggggccgcacctgtgcagagagtgaggaggggcagcaggagaggggtccaggccatggtggacgtgccccgagctctgcctctgagcccccagcagtgctgggctctctgagaccctttattccctctcagagctttgcaggggccagtgagggtttgggtttatgcaaattcaccccccgggggcccctcactcagaggcggggtcaccacaccatcagccctgtctgtccccagcttcctcctcggcttctcacgtctgcacatcagacttgtcctcagggactgaggtcactgtcaccttccctgtgtctgaccacatgaccactgtcccaagcccccctgcctgtggtcctgggctccccagtggggcggtcagcttggcagcgtcctggccgtggactgcggcatggtgtcctggggttcactgtgtatgtgaccctcagaggtggtcactagttctgaggggatggcctgtccagtcctgacttcctgccaagcgctgctccctggacacctgtggacgcacagggctggttcccctgaagccccgcttgggcagcccagcctctgacctgctgctcctggccgcgctctgctgccccctgctggctaccccatgtgctgcctctagcagagctgtgatttctcagcataactgattactgtctccagtactttcatgtccctgtgacgggctgagttagcatttctcacactagagaaccacagtcctcctgtgtaaagtgatcacactcctctctgtgggacttttgtaaaagattctgcagccaggagtcatgggtggtcttagctgagaaatgctggatcagagagacctgataaccgatgtgaagaggggaacctggaagatcttcagttcagttcatttcagtcattcagttgtgtccgactgtttgggatcccatggactgccacacgccagtcctccctgtccatcaccaacttctgaagcttgttcaaactcatgtccatcaagttggagatgcctttcaaccatctcatcctctgtcatccccttctcctcccgccttcaatcttccctagcattagggtcttttccgtgagtcagttcttcgcatcaggtggccaagttttggagtttcagtttcagcatcagtcctttcaatgaatagtaaggactgatttcctttaggatggactggtttgatatccttgcagttcaagggactctcaagagtcttctccaacactgcagttaaaagccatcaattcttcggtgctcagctttctttttggtacaactctcacattcatacatgactaccgaaaatacattagtcgtgtagaaccagtttggggcttcccacgtggctctagtggtaaagaatatgcctgccaactcagaagatgtaagagatgcggttcaatctctgggtcgggaagatcccctggagaagggcatgacaacccactccagtatttttgcctggagaatcccatggacagagaagcctggtggactgcagtccatggagtctcacagagtcagacacgactgaagcaacttagctacttggaaaagagcatgcacgaagctgtctaaaaaacaggtcaagaagtcttgtgttttgaaggtttactgagaaagttgatgcactgctccaacacttcctctcagttgaaaagatcagaagcgttagatcaaatggtggtcaataccttggatgcgctccaacaggttatatctgcagatggaaatgaaggcagtttatggggtaactggaggacaagatgagatcatacacttggaacactgtctggcatcaaaggcgtgtacagtaaacattagctgttattagcaaaataaattcagcttgaatcacccaaatcagatggcattcttaaagccactgagtggtaaaatcaggggtgtgcagccaaaacgtccattttgactcattatgatttccatgtcacaagactagaaagtcactttctcctcagcagaagagaaggtagaacattttaacctttttttggagtgtcaagggaattttgtttacactgtaaagtcagtgaaaatattgaagcttttcatttgtggaaaatattaaatatgtaaaattgaaattttaaaatttattcctgggtagttttgtttttccagtagtcatgcatggatgtgagagttggactataaagaaagctgagcgctgaagaattaatgcttttgaactgtggcactggagaagactcttgagagtcccttggtctgcaaggagatcaaaccagtccatcctaaaggaaatcagtcctgaatattcactggaaggactgatgctgaagctgaaactccaatactttggccacctgatgtgaagaactgactcatatgaaaagactcagatgctgggaaagattgaaggtgggaggagaaggggacgacagaggatgagatggctgaatggcatcaccgactcgatggacatgagtctgaataagctctgggagttgttgatggacagggaggccctggagtgctgcagtccatgggattgcaaagagttggacatgactgagtgactgaactgaactgagtttggtaacagatatgagaattatataatttaaatctaaactcttggtatttctttctttggcggttccaaaagagctgtcccttctgttaactatataaatcctttttgagaattactaaattgataatgttcacaagttatccaatttctcattactcttagttgtcagtataagaaatcccatttgatttatcatgttatagtatctgcaactctaatagttcagttctgacaaatttttattttatttaaaaatattggcatacagtaaaatttcaaacaatatacaattctccctttcagtttaaaaaacaaaacaaaacaaaagtaatattagttaaaaaaatccgggaagaatccaagcatttaaaattgcatcacatttctatgctagacaagctgatataaagttataattaataaaggattggactattaaactctttacatatgaggtaacatggctctctagcaaaacatttaaaaatatgttgtgggtaaattattgttgtccttaaagaaataaaaagacataagcgtaagcaattggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaatggataaggggggaggacatgggtaggggagcgcgatggaggaagtaaggtggtcgagggagttggggggggaataagtgggtaaaagggaagcgggcggaaggagggggaagcaggagagaggggtgggcgtcagatcggggggaggggtatgagggagagggaatggtagacggggggtgggaagcataaaggaaaagataggggggggaaaagttagaagaagaatgaggggataggcggaaagggaagagaaatgggagaagaacagaaaaatagggggagggggggcgtaaagagggggggggagggcaggtgtggagatgacagatacggggaatgccccggtataaaagagtatatggcgtggggcgagaaggctgtcatcctgtgggaggggggacgcggagaacccttcgggctatagggaggattcggggggatcgttcgggaaggcagtcagcacagcacccaccaagggtgcagggatggatctggggtcccaaagaagaggcccaatcccgcgtcttggcagcaaggagccctggagactgggaagtgtccaggacactgacccaggggttcgaggaacccagaagtgtgtctgtgaagatgtgttttgtggggggacaggtccagagctttgagcagaaaagcggccatggcctgtggagggccaaccacgctgatcttttttaaaaggtttttgttttgatgtggaccatttttaaagtcttcattgaatttgctacaatattgtttctggtttatgctctggtttcttcggctgcaaggtttgtgtgatcgtatctcctcaaccaggactgaacccacagcccctgcactggaaggcgaagtcttaacccagatcgccaggaacgtccctcccctcactgatctaatccaagaccctcattaaggaaaaaccgagattcaaagctcccccaggaggactcggtggggaggagagagccaagcactcagcactcagtccagcacggcgccctccctgtccagggcgagggctcggccgaaggaccaccggagaccctgtcggattcaccagtaggattgtgaggaatttcaacttactttttaaatctgtctctcaaggctgttacaagcggactttaccagtaacttaaaagttgaaagggacttcccaggcggcacttgcggtgaagaacccgccggctggttttaggagacataagagatgtgggttagatccctggttcaggaggattcccctggagaaggaaatggcaacccactccagtattcttgcctggaaagcctcacggacagaggaggctggcgggctacagtccacggggtcgcacacgactgaatcgacttagcttcaagttgagacaggaagaggcagtgactggtggcaaaacaccgcacccatgctcccaggggacctgcagcgctctggttcatgagctgtgctaacaaaaatcaacccaacgagaggcccagacagagggaagctgagttcatcaaacacgggcatgatgtggaggagataatccaggaagggacctgccaagcccatgacagaccggtgtcctgtctgagggccgtcctggcagagcagtgcagggccctccgagaccgcccgagctccagacccggctgggggctacagggtggggctgagctgcaaggactctgctgtgagccccacgtcagggaggatcaccttgtttgttttctgagtttctcttaaaatagcctttatgggtcctggtctttggttttaaaataacaactgttctccgtaaacaacgtgaaaaaaaacaaacaggaggaaaacaacgcagcccgggcatttcacccggaagagccgcctctaacactttgacgggttgccttctattttaaccctgttttcattgtaaactgtaaaaaccacatcataaataaattaaaggtctctgtgaagtttaaaaagtaagcatggcggtggcgatggctgtgccacaccgtgaacgctcgtttcaaaacggtaaattctagggaccccctggtggtccagtgggtgagattttgcttccattgcaggagccgtgggtttgatccctggttggggaactaagatcccacatgctgtatggagtggccaaaaagaattttttgtaaatggtgagttttaggtgacgtgaatttcccattgatgcacttcacaggctcagatgcagccaggccctcaggaagcccgagtccaccggtcctttacttttccttagagttttatggcttctgtttctgcccttaaacccaccatgtttcaacctcatctgattttggactttataataaagttaggctgtgtttcaggaaactttgctcagtattctgtaataatctaaatggaaagaatttgaaaaaagagcagacacttgtacatgcataactgaatcactttggtgtacacctgaaactcgagtgcagccgctcagtcgtgtccgaccctgcgaccccacggactgcagcacgcgggcttccctgcccatcaccaactcccggagttcactcaaacacatgtccgtcgactcggtgatgccgtccaaccgtctcatcctctgtcgtccccttctcctcccgccttcaatcttttccagcatcagggtcttttcaaatgagtcagttcttcacaccaggtggccagagtattggagtttcagcttcagcatcagcccttccaacgaccccccatacctgaagctaacacagtgctaatccactgtgctgcaacatgaaagaaaaacacattttttaagtttaggctgtgtgtgtcttccttctctcaacactgcgtctgaccccacccacactgcccagcactgcattccccgtggacaggaggccccctgccccacagctgcgtgccggccggtcactgccgagcagacctgcccgcccagagtggggcccctggcactggggacaaggcaggggcctctccagggccggtcactgtccactgttcctactggttttgttttcaaaagtggaggcagcgtaatatttccctgattataaaaagaagtacacaggttctccacaaataaaacaggggaaaagtataaagaatggaagttcccagcacagcctggagatcacgccgggtgcacctggggtgtccttccaggctggacctcacatttcacgcagacatcagaaggctgcgagatctacccagaaggctgggtagatgggggataggtcagtgacaaacagtagacagagagatatacagacagatgatggatagacagacgctaagacaccgagcgaggggacagacggatggaagacaccatcctttgtcactgaccacacacccacatgggtgtggtgagccggctgtcatacttgtgaacctgctgctctcacaacaccagctgggtccctccagccccagcgtcccacacagcagactcccggctccatccccaggcaggaatcccaccaccaactggggtggaccctccccgcaggaaggtcgtgctgtctaaggccttgagagcaagttacagacctacttctgggaagacagcgcacaaccgcctaccccgcagagcccaggaggacccctgagtcctagggaagggaccacgcggcctggacggggagcggccccaggacgctgcccccaacctgtcccacctcactcctgctctgctctgaggcggggcgcagagaggggccctgaggcctcttcccagttcttgggagcacccactgggcctgaaccaggccagaagccccctcctcaaggtgtccccagaccactcccctccacctccggttgctctgtctcctggcagcagggagccccagtgagaagagacagctccaggctgtgatcttggcccctggctgctctggcagtgtggggggtgggggtcgctgggaggccatgagtgctgggggtcggggctgtgaaagcacctcgaggtcagtgggctgttggtcgggctctgcgaggtccgcacgggtagagctgtgccaggacacaggaggcctggtcagtggtcccaagagtcagggccaaaggaaggggttcgggcccctctggttcctcagcttctgaggccggggaccccagtctggccttggtaggggggcgattggagggtacaacgatccaaaagaaaacacacatctacgagggaagagtcctgaggaggagagagctacacagagggtctgcacactgcggacactgcttggagtctgagagctcgagtgcggggcacagtgagcgaagggaggacggaacctccaaggacaccggacgccgatggccagagacacacgcacgtcccatgagggccggctgctcagacgcaggggagctcctcattaaggcctctcgctgaatagtgaggagaactggccccgtgtgtggggaaacttagcccagaagaaacgctgccctggccccaaggatcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgccctttgcctccagggagggaggaagcgtggatcttgggtttgccttgggtttaaaggatccacccactccctttttagccactccctgtgctggcaatttcttaagactggaggtcgcaaagagttggacacactgagcgagtgaactgcactgagcctaagaaaagtctttgaattcctccaaacaaaacacacttgtcttgggtactttccttggttttgttacaaatgtctggtccctctgttctcctggccagctcctgggtgtcattttgacctgacgaagtcaaagggagcctggaccctcaaaatctgtaggacccagcacccctccattacacctctgttcccccgcgaacgggcacgtgtttcgccgtctggcgtaatgtgtaagcgacggtgtgatactcgggagtcttactctgtttctttttcttctggggtgacaccaccatccgcacgactctgtctgaatgtgaacatttgggtgatttgatgtggcccagactcccccaacgaatgtaccttcaggttggttttcttcttttatattttgcttttgtgaatagacacaggatcccatcagttgtatgtagtgagaaagtaaaaacccactcagccttagctggatggagatctagtagtaagatagcacgttagccggaaatggaaatttcagccagaatctgaaaagcgtgtcctggaaggagaagagggactcaggcccgagcacactgctccacgctggagcctcaggctctgacagctgtacctgccggggtcttcatgggacaggccatgcaggccacgatcccgttgagaagtttcttgcctttccatcacattggcaattgcacgctttgctcttgcttctacatggagttttacttttatcccagacagtttggtttcttctctgattttcgccaattgtacagatcgttacagtatttcttaaccacatagaattcggcagggggggtggggggacagggtagggtggggtgagagtgaggggagggggctgcaccgagcagcatctggggtcgtagctccctgacggggatagacctcgtgcccctgcagtgacagcacagagtcctcctctctgaactgccagggacgctcctgcaattgacttaatgaaaggcatctaattaggaattttggggtgacattttacatttaagtgtgtgagcagtgattatagttcatatcattttatagtttcgtgattttactagcttaaagggtttttggggtttctttttgttttaaaagctaaaatctgttttttaattccatggaatacaaaaaaaaaaagtctgtagaatattttaaagagtgaaggctttgttcggaatgtgagcgctttgctccactgaaccgaacggtaataacatttgtagaagagacgcagagtgaaaggtacctctttttattgagtgacatgacagcacccatcgcgtgagttattggctggagtttagagacaggccatgttgggctaaactccttattgctgttctcagcctttgagtaataatcagaagctttctctgaagagagtggggtcagctgtcagactcctaggtgtctacctgcagcagggctgggattaaatgcagcagccagtagatacgggatggggcaagaggtcaccttgtccctttgttgctgctgggagagaggcttgtcctggtgccagtggggccaaagctgtgactttgtgaccacaggatgtctctgaccctgccttgggttccctgagggtggagggacagcagggtctccccggttccttggccggagaaggaccccccaccccttgctctctgacatccccccaggacttgccccggagtaggttcttcaggatgggcatccgggccccaccctgactcctggagctggccggctagagcttgctgcagaatgaggccttggccattgcggccctgaaggagctgcccgtcaagctcttcccgaggctgtttacggcggcctttgccaggaggcacacccatgccgtgaaggcgatggtgcaggcctggcccttcccctacctcccgatgggggccctgatgaaggactaccagcctcatctggagaccttccaggctgtacttgatggcctggacctcctgcttgctgaggaggtccgccgtaggtaaggtcgacctggcagactggtggggcctggggtgtgagcaagatgcagccaggccaggaagatgaggggtcacctgggaacaggcgttgggtgtacaggactggttgaggctcagaggggacaaaaggcacgtgggcctcccccccagtgtcccttaaagtgggaaccaagggggccccggaagccggaggagctgtggtgtgtggagtgcagagccctcgcggggtcctgatgcccgtcggactctgcacagctcagcgtgtgccccgcggcccggtaggcggtggaagctgcaggtgctggacttgcgccggaacgcccaccagggacttctggaccttgtggtccggcatcaaggccagcgtgtgctcactgctggagcccgagtcagcccagcccatgcagaagaggagcagggtagagggttccaggggtgggggctgaagcctgtgccgggccctttggaggtgctggtcgacctgtgcctcaaggaggacacgctggacgagaccctctgctacctgctgaagaaggccaagcagaggaggagcctgctgcacctgcgctgccagaagctgaggatcttcgccatgcccatgcagagcatcaggaggatcctgaggctggtgcagctggactccatccaggacctggaggtgaactgcacctggaagctggctgggccggatgggcaacctgcgcggctgctgctgtcgtgcatgcgcctgttgccgcgcaccgcccccgaccgggaggagcactgcgttggccagctcaccgcccagttcctgagcctgccccacctgcaggagctctacctggactccatctccttcctcaagggcccgctgcaccaggtgctcaggtgaggcgtggcgccagctccaaagaccagagcaggcctctcttgtttcgtgcccgctggggacattgccagggtgcccggccactcggaagtcctcacgatgccaccgctctgaccctgggcatcttgtcaggtcacttccctggttagggtcagaggcgtggcctaggttaaatgctgtcaaaggggactcctttctgggagtccgcatagtgggggcttggtgtgatgcccttgggaattctttccgagagagtgatgtcttagctgagataatgacagataactaagcgagaaggacggtccatcaggtgtgaggtttgaagtccaaagctctgtctctccctcccacctgccccttctgtcctgagctgttttaggctccaggtgagctgtgggaagtgggtgattctggagatgacaagaagggatcaggaggggaaaattgtggctcctaagcagtccagagaagagaaaaagtcaaataagcattattgttaaagtggctccagtctctttaagtccaaattataattataattttcctctaagacttctgaatacataggaaatcctcagtaacaggttattgctctgccttgaacacagtgataaaagctgggaggatgcagcctaatctgtctgtgtgaatgagttgtattgattccctttttggcagctgcaaactccaagcattaggaataaatatgttcactgagaaccccgaagaaagaaagaaagaaaaaaaaaaagaattgtaggtgttgatggacggtttgtggcccctgaatatctgggggatgttcacccagggatcacgtgtaactgctgggacccccagccccatgtccactgcatccagcctgctgttgaattccgcggatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaattcgagctcggtaccccaaaggtccgtctagtcaaggctatggtttttccagtggtcatgtatggatgtgagagttggactgtgaagaaagctgagtgccaaagaattattcttttgtactgggtgttggagaagactcttgagagtcccttgaactgcaaggagatccaaccagtccgttctaaaggagatcagtcctgaatgttcattggaaggactgatgctgaagctgaaactccaatactttggccacctgacgtgaagagttgactcattggaaaagaccatgatgctgagaggaattgggggcaggaggagaaggggacgacagaggatgagatggctggatggcatcaccaactcgatgngacatgagtttggttaaactccaggagttggtgatggacttggaggcctggtgtgctgggattcatggggtcgcagagtcggacatgactgagcgactgaactgaactgaactgagctgaagagctcacctgtaccagagctcctcaggtcctcctgcaggcctggctgtaatggcccccaggtcaccgtcctgcctccttcatcccatcctttcacgacaggctgggagtggggtgaggtgagttgtcttgtatctagaatttctgcatgcgaccctcagagtgcaatttagctccagagaactgagctccaagagttcattttttccttttcttctttatgatactaccctcttctgagcagagacctcatgtcagggagaaggggactctgccttcctcagccttttgttcctccaagacccacacggggagggtcgcctgcttcactgagccggaaggttcaattgctcatgtcctccagaaacacccccccccccagagacccccagaaataagtggaacagcaccttgtttcccagacaagtgggacacacgttatgaaccacctcagtgattaaaatagtaacctctgtgtatgtgtatttactggagaaggaaacggcaacctactccactattcctgcctagaaaattccatgggagagaagccaggcaggctacagtccacggggtcacagagactgaacatacacaagcacatggaagtgtattttgcagtatttttaaatttgttcagttcaacatggagtacaagaattcaaatcgtgaagtcaattgaccaagaaaccagaagaaatcactgtgttgtgatctctgtggaggtaacatgggtacctgtgctctgaccctcacagcctctggctctctctctacatgtacatacacatatatttccatgtatgtatgtattcggaagatttcacatacgtctcaccagtccacagcccccgcgttccctgatgcccagaacatctgtgatagctgtgagtattgtcaccagataagatcttccaggttcctgcactcacattggttatcaggtctctctgatccagcatttctcagctaagattccttgtgactcctggctgcagaatcttctgcaaaagtcccacagagaggagtgtgatcactgtacacaggagggccgtggttctctagtgtgagaaaagctaactcagcccgtcacagggacgtgaatgtacctgagacagtaatcagttatgctgagaaatcacagctctgctagaggcagcacatggggtagccagcagggggcagcagagcacggccaggagccgcaggtcagaggctgggctgcccaagcggggcttcaggggaaccagccctgcgggtccacaggtgtccagggagcagcgcttggcaggaagtcaggaccggacaggccatcccctcaggactagtgaccacctctgagggtcacatccacagtgaaccccagagcaccatgcctcagtccacggccaggacgctgccaggctgaccgccccactggggagtccaggggagaccacaggccggggggcttgggacagtgatcatgtggtcagacacagagaaggtgacagtgacctcagtccctgaggacaagtctgatgtgcagacgtgagaagccgaggaggaagctggggacagacagggctgatggtgtggtgaccccgcctctcagtgaggggcccccgggggtgaatttgcataaacccaagccctcactgcccccacaaagctctgagagggaataaaggggctcggagagcccagcactgctgcgggctcagaggcagagctcggggcgcgtccaccatggcctgggcccctctcgtactgcccctcctcactctctgcgcaggtgcggccccccagcctcggtccccaagtgaccaggcctcaggctggcctgtcagctcagcacaggggctgctgcagggaatcggggccgctgggaggagacgctcttcccacactccccttcctctcctctcttctaggtcacctggcttcttctcagctgactcagccgcctgcggtgtccgtgtccttgggacagacggccagcatcacctgccagggagacgacttagaaagctattatgctcactggtaccagcagaagccaagccaggccccctgtgctggtcatttatgagtctagtgagagaccctcagggatccctgaccggttctctggctccagctcagggaacacggccaccctgaccatcagcggggcccagactgaggacgaggccgactattactgtcagtcatatgacagcagcggtgatcctcacagtgacacagacagacggggaagtgagacacaaaccttccagtcctgctcacgctctcctccagccccgggaggactgtgggcacagcagggacaggcctggcccggttcccccggagctgagcccccaggcggccccgcctcccggccctccaggcaggctctgcacaggggcgttagcagtggacgatgggctggcaggccctgctgtgtcggggtctgggctgtggagtgacctggagaacggaggcctggatgaggactaacagagggacagagactcagtgctaatggcccctgggtgtccatgtgatgctggctggaccctcagcagccaaaatctcctggattgaccccagaacttcccagatccagatccacgtggctttagaaaggcttaggaggtgaacaagtggggtgagggctaccatggtgacctggaccagaactcctgagacccatggcaccccactccagtactcttccctggaaaatcccatggacggaggagcctggaaggcttcagcccatggggtcgctaagagtcagacacgactgagcgacgtcactttcccttttcactttcatgcattggagaaggaaatggcaacccagtccagtgttcctgcctggaaaatcccagggacaggggagcctggtgggctgccatccatggggccacacagagtcagacacgactgaagcaacttagcagcagcagcagcagcccaataaaactcagcttaagtaatggcatctaaatggaccctattgccaaataaggtccactcgcgtgcactctgtttaggacttcagttcctgattgtggagggttcccacaagacgtgtgtgtatattggtgttgccggaaaacagtgtcaatgtgagcatcccagactcatcaccctcctactcccactattccattgtctctgcaggtattaagcataaaggttaagggtcttattagatggaagaggagtgaatactcgtctgtgcttaacacataccaagtaccatcaaggtccttcctatttattaacgtgtgttttaatcagaaatatgctatgtagaagcatccggacgatagcccatgttacagacggggaagctgaggcatgaagttctcagcaccttgtttcacgtcagacctgaaacggggcagagccggcagcaaacaaggttcctcttcccaagcgcccgctcttcacccgcttcctatggcttctcactgtgcttcctaaactaagctctccccaaccctgtggagacaggattagagactttaggagaaaagaccaggaacatcccacacccgacccgagtgagccactaagacaaggctttgtaaggacagaaccagcaggtgtcctcagcgagccagggagagacctcgcaccaaaaacaatattgtagcatcctgaccctggacttctgacctccagaaatgtgaaaaagaaacgtgtggggtttaatcaactcaccggtgttatttggttatgactgcctgagttaagaaggagttgggaacacttgagtgtaggtgtttatggaacataagtcttgtttctctgaaataaattcccaagggtataattcctaggttgtagggtaactgccacaaatctaggcagcttattaaaaaacaaagatatcactttgccagcaaaggttcatatagtcaaattatggtttttatagtagtcatgtatggatgtaaaagttggatcataaagaaggctgagcaccagagaattgatcccttcaaatcgtggtgctggagaagactcttgagagtcccttggacagcaaggagatccaaccagtcaatcctaaaggaaatgaactgtgaatattcactggaaggactgatgctgaagctgaagatccaatactttggccacctgatgcgaagagttgactcattggaaaagaccctgatgctggaaagcttgagggcaggaggagaagagggcggcagaggatgagacggttggatggcatcactgactcaatggacatgagtttgagccaactctgggagacagtgaaggatagggaaggctggcgtggtacagtgcatgcggtcacaaagagtctgacacatcttagtgactcaacaacgacagcaacacaggcatcacacgcttagtgtgataagcggcagaactgttttccaggggtccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtacgattcgagctcggaccctgacattgtgagtcacgtcatgagcagctgttttccggtcttcagggattgtggacgatttctgtttgggtttgctcatgataatttagttacagcttaggttctttctttccaggccacgagcgacatgttttcaggtgagatgacgtggtgggggatgggcggccaagcccccactggggggggagggattctgttgtgggcaggagttggcagcatccctgaactgatgacctgcgatccaggtgacaagaaccgggggatattattcctctgccttctcatgtcatgtcctcggttcttcatgatgaaaacatatgacaatacaggggagttagatttgggcgggcacaactctgggtgggggacccggtggcattgtgcccagcagggccatcaagatgagggcgacctgggtggtccccttctcccctggggtcttagttttcccctcatggaaatgggatcaggcagcagccatggaacaccgcgaccgtggcttctctcacctcctcgtctgtgattttgggtcgggataccaggcatgaagacctggggcggggggacatcactcctctgcagcagggaggccgcagagtcctccgtccatgaggacttcgtccctgggctgaccctgcggactgctggaggctgaagctggaggcacaggcgggctgcgaggccagggtcctgaggacgacagagccagtggggctgcagctctgagcagatggcccctcgccccgggccctgagcttgtgtgtccagctgcaggttcgctcaggtgagccactacgttatgggggaggcgccctgggcagggatcgggggtgctgactcctccgagattccgaccttctgggagcactctggccacactctaagcctggcaagagctgggttcatcagtctaactctcctcctgaagtccaatggactctctccatgcggcagtcactggatggcctctttatccccgatggtgtccttttccgctgacctggctctcctgaccacctcccagccccccaccatacaggaagatggcacctggtccctgcagagctaagtccacccctggcctggcttcagatgcctacagtcctcctgcgggaggccccgctccccactaggccccaagcctgccgtgtgagtctcagtctcacctggaaccctcctcatttctccccagtcctcagctcccaaccccagaggtatcccctgcccctttcaaggcccttgtcccttcctggggggatggggtgtatgggagggcaagcctgatcccccgagcctgtgccgctgacaatgtccgtctctggatcatcgctcccctggctctcagagctccctggtccctggggatgggttgcggtgatgacaagtggatggactctcaggtcacacctgtcccttccctaaggaactgacccttaaccccgacactcggccagacccagaaagcacttcagacatgtcggctgataaatgagaaggtctttattcaggagaaacaggaacagggagggaggagaggcccctggtgtgaggcgacctgggtaggggctcaggggtccatggagaggtgggggagggggtgtgggccagagggcccccgagggtgggggtccagggccctaagaacacgctgaggtcttcactgtcttcgtcacggtgctcccctcgtgcgtgacctcgcagctgtaactgcctttcgatttccagtcgctgcccgtcaggctcagtagctgctggccgcgtatttgctgttgctctgtttggaggcccgggtggtctccacgttgcgggtgatggtgctgccgtctgccttccaggccacggtcacgctacccgggtagaagtcgctgatgagacacaccagggtggccttgttggcgctgagctcctcggtggggggcgggaacagggtgaccgagggtgcggacttgggctgacccgtgtggacagaggagagggtgtaagacgccggggaggttctgaccttgtccccacggtagccctgtttgccttctctgtgccctccgacccttgccctcagcccctgggcggcagacagcccctcagaagccattgcaatccactctccaagtgaccagccaaacgtggcctcagagtccccggctgcgaccagggctgctctcctccgtcctcctggccccgggagtctgtgtctgctcttggcactgaccccttgagccctcagcccctgccagacccctccgtgaccttccgctcatgcagcccaggtgcctcctccgtgaacccgggtccccccgcccacctgccaggacggtcctgatgggagatgtggggacaagcgtgctagggtcatgtgcggagccgggcccgggcctccctctcctcgcccagcccagcctcagctctcctggccaaagcccggggctcctctgaggtcctgcctgtctaccgtccgccctgcctgagtgcagggcccctcgcctcacctgccttcaggggacggtgcccccacacagcacctccaaagaccccgattctgtgggagtcagagccctgttcatatctcctaagtccaatgctcgcttcgaggccagcggaggccgaccctcggacaggtgtgacccctgggtcccaggggatcaggtctcccagactgacgagtttctgccccatgggacccgctcctttctgaccgctgtcctgagatcctctggtcagcttgccccgtctcagctgtgtccacccggcccctcagcccagagcgggcgagacccctctctctctgccctccagggccttccctcaggctgccctctgtgttcctggggcctggtcatagcccccgccgagcccccaagctcctgtctggcctcccggctggggcatggagctcacagcacagagcccggggcttggagatgcccctagtcagcaccagcctctggcccgcaccccagcgtctgccctgcaagaggggaacaagtccctgcattcctggaccaaacaccagccccggcgccccgactggccccattggacggtcggccactggatgctcctgctggttaccccaagaccaacccgcctcccctcccggccccacggagaaaggtggggatcggcccttaaggccggggggacagagaggaagctgcccccagagcaagagaagtgactttcccgagagagcagagggtgagagaggctggggtagggtgagagccacttacccaggacggtgacccaggtcccgccgcctaagacaaaatacagagactaagtctcggaccaaaacccgccgggacagcgcctggggcctgtcccccgggggggctgggccgagcgggaacctgctgggcgtgacgggcgcagggctgcagccggtggggctgtgtcctccgctgaggggtgttgtggagccagccttccagaggccaggggaccttgtgtcctggaggtgccctgtgcccagccccctggccgaggcagcagccacacacgcccttggggtcacccagtgccccctcactcggaggctgtcctggccaccactgacgccttagcgctgagggagacgtggagcgccgcgtctgtgcggggcggcagaggagtaccggcctggcttggacctgcccagccgctcctggcctcactgtaaggcctctgggtgttccttccccacagtcctcacagtccagccaggcagcttccttcctggggctgtggacaccgggctattcctcaggccccaagtggggaaccctgccctttttctccacccacggagatgcagttcagtttgttctcttcaatgaacattctctgctgtcagatcactgtctttctgtacatctgtttgtccatccatcgatccaacatccatccatccatccatcacccagccatccatctgtcatccaacatccatccttccatccattgtccatccatctgtccatcttgcatctgtctgtccaacagtggccatcaagcacccgtctgccaagccctgtgtcacacgctgggacttggtggggggagccctcgccctcccaccctcccatctctcctgaaacttctggggtcaagtctaacaaggtcccatcccgtctagtctgaggtccccccgcagcctcctcttccactctctctgcttctgacccacactgtgcactcggacgaccacccagggcccttgcatccctgtttccttcctgacctcttttttttggctctggatttatacacattctgcctcctggaggcgtctcagcttgagtgtcccacagacgcctcagactcagcatcttccatcgaaactgctcccaggtccttgcagacctggtcccccacattgttctcaattcggtagatttctccacaagccagaggcctggactcatcccataatgcctgcccctcattgagtcagcctctgtgtcctaccataaccaaacatccccttaaaaatctcagaagaacaaaaaaagcacccagatggcactgtcagagtttatgatgacaagaatcctcagttcagttcagtcactcagtcgtgtccgactctttgcgaccccatgaatcgcagcacgccaggcctccctgtccatcaccaactcccggagttcactcagactcacgtccattgagtcagtgatgccatccagccatctcatcctctctcgtccccttctcctcctgcccccaatccctcccagcatcagagttttttccaatgagtcaactcttcgcgtgaggtgaccaaagtactggagtttcagcttcagcatcattccttccaaagaaatcccagggctgatctccttcagaatggactggttggatctccttacagtccaagggactctcaagagtcttctccaacaccacagttcaaaagcctcaattctttggcgctcagccttcttcacagtccaactctcacatccatacatgaccacaggaaaaaccataaccttgactagatggacctttgttggcaaagtaatgtctctgctttttaatatgctatctaggttgctcataactttccttccaagaagtaagtgtcttttaatttcatggctgcaatcaacatctgcagtgattttggagccccaaaaaataaagtctgccactgtttccactgtttccccatctatttcccatgaagtgatgggaccagatgccatgatctttgttttctgaatgttgagctttaagccaacttttcactctccactttcactttcatcaagaggctttttagttcctcttcactttctgccataagggtggtgtcatctgcatatctgaggttattgatatttctcctggcaatcttgattccagtttgtgtttcttccagtccagtgtttctcatgatgtactctgcatataagttaaataagcagggtgataatatacagccttgacgtactccttttcctatttggaaccagtctgttgttccatgtccagttctaactgttgcttcctgacctgcatacagatttctcaagaggcaggtcaggtggtctggtattcccatctctttcagaattttccacagttgattgtgatccacacagtcaaaggctttggcatagtcaataaagcagaaatagatgtttttctgaaactctcttgctttttccatgatccagcagatgttggcaatttgatctctggttcctctgccttttctaaaaccagcttgaacatcaggaagttcacggttcatgtattgctgaagcctggcttggagaattttgagcattcctttgctagcgtgtgagatgagtgcaattgtgcggcagtttgagcattctttggcattgcctttctttgggattggaatgaaaactgacctgttccaggcctgtggccactgttgagttttcccaatttgctggcatattgagtgcagcactttcacagcatcatctttcaggatttgaaatcgctccactggaattccatcacctccactagctttgtttgtagtgatgctctctaaggcccacttgacttcacattccaggatgtctggctctagatgagtgatcacaccatcgtgattatctgggtcgtgaagatcttttttgtacagttcttctgtgtattcttgccacctcttcttaatatcttctgcttctgttaggcccataccgtttctgtcctcgcctatcgagccctcgcctccctacgtagagactctaagcaggaaggtgacccgtgctgcactgggtccagcatgcttttaattcagcagtggaacttctgggtcatgattgtgtttaagggatgcgcatacgatttttgaagcaaaatttaacaggacagcagtgtaaagtcagtacttatttctgattaaagaaagcaaatatccagcctgttactaagttaattaactaaagaaacatcttcaacttaataaacagtatctcctgaaacttacagcatgcttcacatttaaaggcaaaaccattttagaggccagggttcccacgcttacgtttattatttaatatatgctacagattcaagcccatgacacaaaatggggggaagagtgtgagtgttaggaaaaatgagataaaattggtttttgcaggtgatgggctagtttactttaaaaaaaaaaacaaaacaagctcaagatgaactgaaggactattagaactggtacaagagttaacctgtgatcgaatacaagcaggctgggcaaaactcagcaggttttcttctatacaggcagtaatgattgagaatacgaaacggcggaagcgcttacaacctcgataacagttctattaaaagccctaggaatgaacttaacacggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctccccccaccctcccctcctccccccccaccaccagtgccccaggtctcgtgcccagagagctgaagatgccagcaggcccgctgcctgcctcgctcgcgtggcccgggctcgctgccggtctgcctgcccagcacacagatgcagccccagctctcgctgccacccgcctcccccaggcaggactctcccacaacaccaagggcgtctctgggttcaggatggccctcgttgaggtgtaaagtgcttcccggggctgagacgaatgggccggagatccaaacgaggccaaggccgccacggcgcctggcgcagggcacccatggtgcagagcggcccagctccctccctccctccctccctccctgcttctttatgctcccggctatgtctatttttactctgcaatttagaaatgataccgaaggacaaacaccgttccccctgtgtgtctgctctaaaccctttatctacttatctattagcgtgtccaagttttgctgctaagtgaatgaaggaacactacccacaagcagcaacgtccccacgaccctcgcctgttcaactgggaatgtaaatgtgctttcaaaggacctaagtttctatgttcaaaaccgttgtgtgtttcttttgggagtgaacctaggccactcgttgttctgcctttcaaagcattcttaacaactctccagaacccagggcttggcttacgtttccagaaattccaaagacagacacttggaaacctgatgaagaaggcctgtgagcacagcaggggccggggtacctgaggtaggtggggggctcggtgctgatggacacggccttgtacttctcatcgttgccgtccaggatctcctccacctcggaggctttcagcagggtcacgctggtggccagggtcgtgtatccatgatctgcaaccagagacggggctgcggtcagcccgcgggcgggcagcaggcaggagcagccaggagacgcagcacaccgaggtcctcacatgcaggaggtgggggaagcggctgtggacctcacgactgcccgatgtgggcctcttccaaagggccggcctggaccctggctttctccagaggccctgctgggccgtccgcacaggctccagccacagggcctcttgggacaggagggctccagagtgagccggccggcgggaagaggtctgacaccgctgcagtccacaacacgaagcgaggtggagatgggatgagggatgagaaacacttttcttttaaaacaagagcccagagagttggaaagagctgctgcacacgcaacatgaactcctggccccggtgccagcggcgctgggagcccgagttctcggcaatccgaccacagcttgcctagggagccgggtggagacggagggttaggggaaggcggctccccagggagcgcgaggcccggggtcgccaaggctcgccaggggcaagcgcagctaggggcgcagggttagtgaccggcactgcacccggcgcaggagggccagggaggggctgaaaggtcacagcagtgtgtggacaagaggctccggctcctgcgttaaaagaacgcggtggacagaccacgacagcgccacggacacactcataccggacggactgcggagtgcacgcgcgcgcacacacacacacacaccacacacacacacacacggcccgggacacactcataccggacggactgcggagtgcacgcgcacacacacacccaccacacacacacccaccacacacacacccaccacacacacacacacacacacacacccccacacacacccacacacacccacacacacccacacacacacacccacacacacacacacacacacacacacacacacggcccggtggccccaggcgcacacagcacggagcaaacatgcacagagcacagagcgagcgctagcggaccggctgccagaccaggcgccacgcgatggattgggggcggggacggggaggggcgggagcaaacggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtattaaagaagccgggagcgagaatatgacggcaagaggatgtaggtgggggcggggcaagagtaaagagagcggacggtagaggggatgcgattgtgatgcggaagcgagacgaggagtgatgccgtattagattgatagcaagaggaacagtaggaggggggggggagaggagggggaggtggggggtggtgggtgggaagggaactttaaaaaaaagaggggagagttggaggggggaataaacgggcggtaaaaaagaacaatttgaaattaccagggtggggcggccaggggggtgattcattcttggagggggcaacatatggggggtggctgtcgcggattaggagaaaataaatatcaggggtgattaagtgtttggcgttggggaataatgaagtaagaatcaaatatgaatcgcgttggcatcgttagccatcgggggaaacatttcccatgcaaggaacaaggatgtgagaatgcgtccgtctgaaccaccgtcccggggtcccagtaggactcgccgagctgatagttgccggagcaacagttaagggagcagaagctgctacaaaaccaccacctgccaaagtagggtctccaattacggagtgcgcctcctgggtgtcggtccaaacctttggaaaggacctggaaataagtgctacccaccagatattaatataaacccacctggccaggagaggcaggcgctgctggcacaggaagtgtccccagactcagtcatcaaggtaaataatattttgggacctccctggaaatccagtggttaggactctgcggttcaatccctggtcggggaactaagatcccacaagtcacaagacatggccaaatttaaaaaagaaaaaaagagagagaaatatttagtgcaataggttttagaattgaaattaagctcctgcccacccccaccccccaatctggatgaataaagcattgaaatagtaagtgaagtcaggctctgacatgcactgatgtgactcaccttaagcaacccccaccctaggactggtcggggttccaggagtttcaggggtgccaggaagatggagtccagcccctgccctctccccccaccacgtcctccactggagccgcctaccccacctcccacccctccgcaccctgctaccccccacccctgcccccaggtctcccctgtcctgtgtctgagctccacactttctgggcagtgtctccctctacagctggtttctgctgcccgctaccgggcccgtcccctctgttcagttcagttcagtcgctcagtcatgtctgactctttgtgaccccatggactgcagcacaccaggcctccctggccatcaccaacccccagaacttactcaaactcatgtccatcgagccagtgatgccatccaaccatctcatcctctgtcgaccccttctcctggcctcaatctttcccagcatcagggtcttttccaatgagtcagttctttgcatcaggtagccaaagtattggagtttcagcttcagcatcatttcttccaatgaatattcaggactcatttcctttgggatgaactggttggatctccttgcagtccaagggactctcaagagtcttctccaacaccacagttcaaaagcatcaattcttcagtgctcagctctctttatagtccaactctcacatccatacgtgaccactggaaaaaccatagcctcgactagatggaactttgtgggcaaagtaatgtctctgcttttgaatatgctgtctaggttggtcataacttttcttccaaggagcaagcgtcttttaatttcatggctgcagtcaccatctgcagtgatttttggagcccaagaaaataaagtctgtcactgtttccactgtttccccgtctatttaacggagggaaatttcccagagcccccaggttccaggctgggccccaccccactcccatgtcccagagagcctggtcctcccaggctcccggctggcgctggtaagtcccaggatatagtctttacatcaagttgctgtgtgtcttaggaaagaaactctccctctctgtgcctctgttccctcatccgcagaagtgactgccaggtcggggagtctgtgacgtctccagaagccggaggattttctccccatttgctgaaagagagctcggggtgggggaagcttctgcacccctaggatcaccagaggagccagggtcttcagggttcccggggacccctcagtgggggctcaggaaccacagagccagaccctgattccaaaaacctggtcacacctccagatgaccctttgtcccttggctccgcctcaaatgctccaagccccaacagtgaagcgcttaagagaaggatccaccaggcttgagtttggggaggagggaagtggggagctgggggagggcctgggcctgggagacaggaatccaccatggcttcaggcagggtctctggggcctgcggggtggagagcgggcaggagcagacagaggtgactggacacgacacacccctccactccaagggaggtgggcaggggcggggcacagaggaacaagagaccctgagaaggggtccaccgagcagactgctggacccagacatctctgagccagctggaatccagctctaagccatgctcagcccaggcagggtatagggcaggactgagtggagtggccagagctgcagctgcatgggctgggaaggccctgcccgtcccctgagggtcccccagggtctagccagactccaatttccgaccgcagcacacacaggaggaagtggtcggggtggagttggcccagaggtctgggcaggtgcagggtgggggaaggggggcagctggagtcacccgctgaattcagggacagtccctttttctccctgaaacctggggctgtcccgggggccaccgcagcctccaggcagcggggggacccagcccccaatatgtgagaagagcaggtcccaggctggagagagcgaagcaccatggtggggagaagttagactggatcggggcccctaggggctcccccggacctgcacggcagccgtcagggcacccgcaccccattgctgttcagtgctggccagtgtccaaggccagggatgtgtgtgtgtgtgtgtgcgtgcgtgcgtgcgtgtgtgtgtgcgtgtgtgcgcgtgcgtgcgtgtgtgtgtgtgtgcgtgcgtgtgcgtgcgtagacgtgtgcgtgcgtgcgtgcgtgcgtgcgtgtgtgtgcgcacgcgcgcagcccagcctcagcactggaccaggcagcctgggattcctccaaaactgccttgtgagtttggtcaaaccgtgaggctctgatcaccgccatccattcgccccctcctgcccccctcatcaccgtggttgttgtcattatcgagagctgtggagggtctgggaggtcatcccacctgccagctaaaccgtgaggctgccgcaatcgcactgatgcgggcagacccgagacgctgtgccggagacgaaggccagcttgtcaccccgccagagcggcagtcgggccacaagcatcatccaagcagtggttctctgagcccgacggggtgatgcaaaggagccaggagacacctgcgcgtccaagctgggggaccccaggtctgttatgccggacagtaaacacgttcagctccggagggagagggttcccctaccttccagggtttctcattccacaaacatccaaagacaatccataccgaaggcgatccgtgcctttgctcctgagacgtgcggaagcacagagatccacagacactgtctcccaggatcctatgtatgtaaaggaaccgaagtcccaggctgtgtgtctggtaccacatcccacggaacaggctggactgattttcaccaaatgtagcagaaacgttaaggagtatcagcttcaaaatatgagggccagacatgtctgagaagtcccttccagaaaagtccctttggggtccttccccagagttgctgaaacagagaaccggaagggctgcagagctgaacttaaacaactggatcgcaaaggtccgtctcatcagagcgatggtttttccagtggtcatgtatggatgagagagttggaccataaagaaagctgagcgccgaagaatcgatgcttttgaactctggtgttggagaagactcttgagagtcccttggactgcaaggagatccaaccagtcaatcctaaaggaaatcaatcctgaatattcatgggaaggactgatgctgaagctgaaactccaatactttggccacttgatgcaaagaactgactcactggaaaaaccctgatgctgggaaaggttgaaggcaggaggagaaggggtcgacagaggatgagatggttgggtggcatcacccacccatggactcaatggacatgggtttgagtaaactctgggagttggtgatggacagagaatcctggcatgctgcggtccatggggtcatagagagtcagacacaactgagcgactgacagaactgaagcaactggcaagccggagggtaggtgccggctgcgatgagcgggaacgtgcaacctgccacgtggagctcttcctacacccagagtcctgacggcactgggaccctagccctccacggcctctccagggccacgagacaccctcacagagcagagaagcggaacagagctggtgtgcagaaccaggccccgggggtggggcggggctggtgggcaggctttagtgagaagcccttgagccctggaaccagagcagagcagaacagttggcagaggcccccctgggagaggccccccgcccagagtaccggccctgggccctgggggagagggcggtgctgggggcagggacagaaggcccaggcagaggatgggccccgtgggacggggcgcaccaaaacagcccctgccagcaaggggaagctggggcactttcgaccccctccaaggaggagcccacaccagcgcatctgcccaaggtgcccttggccctgggggcacatgaggcccaggccaggccagggggcccatgaggcccccaggggtcagtgcagtgtccccaggcagccctggcctctcatcctgctgggcctggcctcttatcccgtgggcgcccacggcctgctgcccccgacagcggcgcctcagagcacagccccccgcatggaagccccgtcaggaaagagcccttggagcctgcaggacaggtaagggccgagggagtcatggtgcagggaagtggggcttcccttcgatgggacccaggggtgaatgaccgcaggggcggggaacgagaagggaaaccagctggagagaaggagcctgggcagacgtggctgcacgcacagcgctgaccctgggcccagtgtgcctttgtgttgggttttatttttaattttgtattgagatgctatttatctcgtggagcttttgccgccctgagattttgtacccgtggctggtgtccctcttgcctcaccccggcctctgtagcagggcagacacggcgcaacggggcagggcgtgcccaggaggcactgtcattttgggggcagcggccccacaaggcaggtctgccttcctcccctcttacaggcagcgacagaggtccagagaggtgaggcaagctgcccaatgtcacacagcacacgggcgcagtcccaggactgtagaaatcccgggactagacaggcaccagagtgtcctgtgtttttaaaaaaacggcccaagagaagaggcaagtctgcaaggcgtcccgggaaggcagcaggggcttggctcggtctcccccaaggaggccagctcctcagcgaggttcctaagtgtctaacggagccaagcctgaaccaagggggtcacgtgcagctatgggacactgacctgggatgggggagctccaggcaaagggagtagggaggccaaggaggagagaggggtgcacaggcctgcagggagcttccagagctggggaaaacggggttcagaccacggggtcatgtccacccctcctttatcctgggatccggggcaggtattgagggatttatgtgcggggctgtcagggtccagttcgtgctgtggaaaaattgtttcagatcagagaccagcgtgaggtcaggttagaggatggagaagaagctgtgaaaaggtgatggagagcggggggacggtcctcggtgatcaggcaccgagatcgcccatggaatccgcaggcgaatttacagtgacgtcgtcagagggctgtcggggaggaacaggcactgtcatgaactggctacaaaaatctaaaatgtgcacccttttcggcaatatgcagcaagtcataaaagaaaacgcatttctttaaaattgcgtaattccgcttttaggaattcatctgggggcgggggaacaatcaaaaagatgtgaccaaaggtttacaagccaggaagtcaactcgttaatgatgggagaaaaccggaaataacctgaatatccaacagaaagggtgtgatgaagcgcagcatggcacatccaccgcaaggaatcctaacacaaacttccaaaacaatatttctgacgttgggtttttaaagcatgcgtgcactttcaaaagcttgtcagaaaacatagaaatatgccaataatgtgtctctagccaaattttttaatttttgctttataattttataaagttataattgtatgaaatataatgataaaattataaactataaaaaagttatgaaaatgttcacaagaagatatacatgtaattttatcttctacaatactttttaataccagaataacgtgcttttaaaaaagattgagcacagaagcgtataaagtaaaaattgagagtttctgctcaccaaccacacgtcttaccttaaaacccattctccagcgagagacagtgtcatgtgggtctgtacacttctggcctttctcctaggcatgtatgtccctgaaaactcacacacacggctaatggtgctgggattttagttttcaaaacggactcatactctgcctatgagcctgcaactatttattcagtctgttgagattttctatatcagcccacatggatcccgcatgttctctgaatggctctgtatgaattcaaagtttggaagaagcagcgtgtctttaatcattcgcctattaatggacgtttggggtgtttccactacaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngatacaattcgagctcggtaccctggcttgaactatatgaacagagaacgatgagaacagtttctcaaacttggaacagttaacattttgggctaaatgattcttttttgtgtggagttggcctatgaatagaggatattagcagcatcatttaacctttactcactacatacctgtagcaactacatcctctccatttgtgtcaatcaaaactgtctccggacatggacaagtgtgcccctgggatgggtggaatgaccttttgttaagaaccactgggtcagagattcatagatttttgtcttgttgactttttaaaaatacatcttggtttttattttattggtttctgctcttatctttatgattaccttccttttacttggggcttccctgatagattttcccttctggctcagctggtaaagaatctgcctgcaatgcaggagacctgggttcagtccctgggttgggaggatcccctggagaggagaagggctacccaccccagtattctggcctggaggattccatggagtgtatagtccatggggtcgcagagtcggacatgactgagtgactttcacacacacatatgtccctggtagctcagctagtaaagaatcccacccgcaatgcaggagaccccggtccaattcctgggtccggaagattcccttttgtttactccataagatcttatctggggacaaaactaacagctatgccagaccttctggacatcagggaacgtgaggggtgtggactggacagatgtgtgtgttctcccaaacacaaacatacatctgtatacatgtacatggagagagggggagggaggctgtgagtctccaggggaccgtgcaaccatgtgacattcatggaggcgtttgcgggtgatcactacacagtttcttcttctggtttcttggtcaattgacttcacaattccaattcctatacttcattttagactgagggaattttacactattgtaagacatatgtatacatgagttatgttcagcgccatgagggctcattttgtgtgtccactttgcctggaaacaaagttggactgatttacttctaggggtgcctgggggtgtttctggaggacaggagcatttgaacccaagggctcggtgaagcatgagcctctctgcaggtggacccaggaggaacgcaaggccgaggaaggcagactctcctcctccctaacccgaggtctctgctcagaaaagggacaatataatgactagaagaaaagaaagaacatcagctgtgggaggtttgttctctggagcagattcacacgttgaggctcatgtgcaggaattctaggtgaaacagagcagtcacccatgtgtgttggaaaattttaaattacatttgcagttacgactttgtttaagccagacagggtagcacagcaaagtcaccatgtggtcacctgtgttttgtaaaggagagagaacttgctggcacattcaggaaaggccgtgtctcagctttggaggcacactgagaggccacaagcagatggtgaggaccagggtctcgggcagagggatcaattcactgctcttcacttttgccacatctgtgtgctgtccatcctggccagagtagttcagtcttcagatgctggagttcccattggtagaaatccaatctgggtcatttttaaacctctcttggttctacttaatggttttaaaatctctttggctcaagaaaaaaaataaacataattttaaagggtggtttggggccttgactataaagtacattatctgggccatttcagagcatggttgaattaatacatttcgtgcttactatagctcctattttcttgattctttacaggtaatttttgttaggaatcgggtactgtgaatattttcttgttgaatacgggatctttgtattttttcctaatttttttttttttttcatttttggttttaccttcaggaaagtcactaggactcaggaaagtcctttgtccgcctgttatttcagtctcttacctggggccagggcagcgtttcctctgggctaagtttccccacaaccggggccagttctcctcactcttcaccctgaggccttaatgaggagctcccctgcgtctgagcagccggccctcctgtgacgtgcgtgtgtctctggccatcggcgtccggtgtccttggaggttccgtcctcccttcgctcactgtgccccgcactcgagctctcaggctccaagcagtgtccgcagtgtgcagaccctctgtgtagctctctcctcctcaggactcttccctctagatgtgtgttttcttttggctccttggacctccgctctgaacgcaggcctggtgctgagtgtgatctctggagggaagcctgggaggctggacgggtccgccctgcggtgtggtgacaggtgtgggctcggggcggggcctgcacgtcgtcctgacccgagccgggactgggctccgggcctcaggcatcactgactgaatctccctcacagaggggtcagggcctgggcgggggaaccgtctctgcaatgacagcccctcccagggagggcacagcggggagctgccgaggctccagccctagtgggaggtcggggagcccaggggagcggcctgacggccccacaccggcccagggctggttcgttctgtttctcgagctcaacagaagctccgaggagctgggcagttctctgaattcgtcccggagttttggctgctgagtgtcctgtcagcaccgtatggacatccagagtccattagcagtggtctctgtccctctgtctgtccttcatcaggctctttgtccaggtcaccacacggccaacaccaggacagtctggtcccgccagcccatcgtccctgcggacgcccctgtgcagcctgccgaagggccgggaggccgggggaaccgggccaggcctgtccctgctgtgtccacagtcctcccggggctggaggagagcgtgagcaggacgggagggtttgtgtctcacttccccgtctgtctgtgtcactgtgaggattatcactgctgtcagctgactgacagtaatagtcggcctcgtcctcggtctgggccccgctgatggtcagcgtggctgttttgcctgagctggagccagagaaccggtcagagatccctgagggccgctcactatctttataaatgaccctcacagggccctggcccggcttctgctggtaccactgagtatattgttcatccagcaggtcccccgagcaggtgatcttggccgtctgtcccaaggccactgacactgaagtcggctgggtcagttcataggagaccacggagccggaagagaggagggagaggggatgagaaagaaggaccccttccccgggcatcccaccctgaggcggtgcctggagtgcactctgggttcggggcaggccccagcccagggtcctgtgtggccggagcctgcgggcagggccggggggccgcacctgtgcagagagtgaggaggggcagcaggagaggggtccaggccatggtggatgcgccccgagctctgcctctgagcccgcagcagcactgggctctctgagaccctttattccctctcagagctttgcaggggccagtgagggtttgggtttatgcaaattcacccccgggggcccctcactgagaggcggggtcaccacaccatcagccctgtctgtccccagcttcctcctcggcttctcacgtctgcacatcagacttgtcctcagggactgaggtcactgtcaccttccccgtctctgaccacatgaccactgtcccaagccccccggcctgtggtctcccctggactccccagtggggcggtcagcctggcagcatcctggccgtggactgaggcatggtgctctggggttcactgtggatgtgaccctcagaggtggtcactagtcctgaggggatggcctgtccagtcctgacttcctgccaagcgctgctccttggacagctgtggacccgcagggctgcttcccctgaagctccccttgggcagcccagcctctgacctgctgctcctggccacgctctgctgccccctgctggtggaggacgatcagggcagcggctcccctcccgcaggtcaccccaaggcccctgtcagcagagagggtgtggacctgggagtccagccctgcctggcccagcactagaggccgcctgcaccgggaagttgctgtgctgtgaccctgtctcagggcggagatgaccgcgccgtccctttggtttgttagtggagtggagggtccgggatgactctagccgtaaactgccaggctccgtagcaacctgtgcgatgcccccggggacccagggctccttgtgctggtgtaccaaggttggcactagtcccaccccaggagggcacttcgctgatggtgttcctggcagttgagtgcatttgagaacttacatcattttcatcatcacatcttcatcaccagtatcatcaccaccatcaccattccatcatctcttctctctttttcttttatgtcatctcacaatctcacacccctcaagagtttgcattggtagcatatttactttagcacagtgtgcctctttttaggaaactgggggtctcctgctgatacccctgggaacccatccagaaattgtactgatggctgaacccctgcgtttggattcttgccgaggagaccctagggcctcaaagttctctgaatcactcccatagttaacaacactcattgggcctttttatactttaatttggaaaaatatccttgaagttagtacctacctccacattttacagcaggtaaagctgcttcgcatttgagagcaagtccccagatcaataaagagaatgggatgaacccaggatggggcccaggggtcctggattcagactccagccgtttaggacagaacttgactaggtacgaagtgagcggggtgggggggcaatctggggggaactgtggcacccccagggctcggggccatccccaccacatcctggctttcatcagtagccccctcagcctgcgtgtggaggaggccagggaagctatggtccaggtcatgctggagaatatgtggggctggggtgctgctgggtcctaggggtctggccaggtcctgctgcctctgctgggcagtgataattggtcctcatcctcctgagaagtcacgagtgacaggtgtctcatggccaagctattggaggaggcagtgagcactcccacccctgcagacatctctggaggcatcagtggtcctgtaggtggtcctggggcttgggccgggggacctgagattcagccattgactctcagaggggccagctgtgggtgcagcggcagggctgggcggtggaggatacctcaccagagccaaaataagagatcacccaacggatagaaattgactcacaccctttggtctggcacattctgtcttgaaatttcttgtggacaggacacagtccctggataaagggatttctatcttgcgtgtgcaatagagctgtcgacacgcttggctgggacatgtaatcctttgaacatggtattaaattctgttcactaacatctgaaaggatttttgcatcaataaacctaaggtatattgccctgtcatttccttgtcttgtagtgtctctgagtaggctggaaggggtaaccagcttcacaaatcgagttaggaaattcccttattcttccactgtctaatagactttcataagattagtgttaattcctctttaaatcgctgctataatcatcactgtggccaccggtactgaattttttgttaggatgatttttaaacaagcattttaatgatttttccttttattttcggctgtgctgggtctcgttgctgtgtgccggcgttctctcgctgtggccagtgggggcgctgctctcgcgttgcgaagctcgggcttctgactgcagtggcttctctcgttgcagagcgcgggctccagggcgctcaggctcgcgtggctgcggcacgtgggctcagtagtcctggggcacaggtgcagcagcctctcaggacgttttgttcccagatggtgggtcggtcgaaccggtgtcccctgcgttgcaaggtggattcttcaccgctggaccaccagcgacgttccctggaggtttttaattatggatttaagctctcattagatgtctcctcacatttcctatttctttttgagtcagtttgatactttgtttgtgtctgtaagtttgtccattttatccaagtcatctaatgtgttgatagacaattattggttagtcatctaattgttggtttacaattttgagagcattgtcctgcaattccttctatctgcaagattggtaataatatctcccaagaggagtcacaaactgaaatgagattanatacaggctttttttttaaaagaatgaacttatgttgttgcctttctcatagatcttacttcttagcatgactgtacttactgactggggcgttttcatgtctgtgtggagagctaccattagtacttcttatcgcccaaagacatcgggctcctgggcacagtgaaaacactcctttctgtggctattttgcaaaatatggcctagcctagcgtcataagggatcacagctgacaactgctggaacagagggacatgcgaagcaacgtgagggctggaacctggagggtcctctctggggacagtttaaccagctataatggacattccagcatctgggacatggagctgtgaactggaccaatgactgtcatttttggaagagaaatcccaggagagaagggtccaggggaatctgaggccgcatgcagtgcctcaggacaggggacaccttctccagcagagcaggggggcccgcccaggccgcctgcagtgattccaccaggaggagatgcatccctgcagacctctgacagcacggccctctcctgagacacagggtcacacccggggccctggaaccctttgagaccctaaacctttcctttcctgaccaccctgacagcagtctagctcagaacagacatcttcattttcagcaggaaaatccttttcctcgtttgagggagcgactggcaccggaggagctgagtcttttaaacacaggctgcctgaacctcagggatgacctgcagctgctcagaggaggctggagtgtgatagctcactctaatgttactaaaaggaacatattggacaccccctctctgaaaaatttccctcctgcctctcatctcttagtccactttatcgccgttttactgcttttctatttactactcttaacgccaacctatcttatttcccctcccagtttaacacggttttccctccacccgctctctttaatctcagaagattctgcctattcctctattatcacacgcccctactttttattttttttcttacccgccttttattccctcccctcctcactctctatttaattacatcttaactacaccgcctgcgctatcttcgaatgtatccaaatatttttcccttatataacactccaggccgagcggctaacttattataatttctttatagcgcctacctaatttccctttatttctaattatctatatatacccatgcaatttcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgggtgtacgttatagagtaaacgcgcatgaagaagtgggtcaatctatggctgtgagaggcagaaaataatattatcatatataatttatgttataacacactgaggtggtgggctcgtagaatagtgcggacggggagaaaggtgggaaggagaagacacaagagagagatgttcgcctcgcgggatggatgggcggagggatagaagaataaaaagaggagaggtatagaggggggcggggggcataacgtgtggtggggtaaatagtaggcggtaattatgaaaaaaagaaagacggggggggcggtaacatagaatacgcaaaaaagtcatatactgaacggggattagggagaagaggtggggggcgtggggtgcgggggaaagaggtgtgtgtataattggtatggagtgttatttgaatatatattaatgtaatagggagtgtaattagtgaaattgtgggagtattatattggggtgtgggggacatggcaaagtgatgatcgggataaaaaaagtaaagcaagaggggaggggaaaataaggggggggagaaggtcgaagaaaataagaggaagaagaaagaacgggggtggcgggcggggggggcgccgctcttgtatctggcttttttgttgtgtcggtggttgttcgcgtcttgttgggtccggggcgggtgtgcggaaaaaaaaaaaggcgggaggcccggggcccggtcacgcggcacccccgcgggtccctggcttctccttcggcagctccgggggtcggtgagcctgcgccctccgggccgccggcccgagctgtgtgcgccctggagaatcggagccgctgtggcagcacgcggagggcgcgcgcaagggccacgggacggaccttcaaaggccgcggcggagcgcggcaagccgaaccgagggcggtctggcgatcggccgagccctgctcccccctcccgcgtggccccagggtcgcgggtggactggggcgggtacaaagcactcacccccgtcccgcccccagaaagcctcccaggactctcacagagcacccgccaggaggcatccggttcccccctcggctcagttcagttgctcagtcgtgtccaactctttgcgaccccatggactgcagcaccccaagcttccctgtccatcaccaactcccggagtttactcaaactcatctattgagtcagtgatgccatccaaccgtctcatcctctgttgtccccttctcctcccactttcaatctttcccagcatcagggtcttttcttatgagccagttcttcacatcaggtggtcagagtattggagtttcagcttcagcatcagtccttccaatgaacactcaggactgatttcctttaggatggactggctggatgcagcgccagacaccgaccgcgtttaccccgtgtgtcctttccaatggctgtcccctgcgggcctaggggcattggtgcgggtttgaatcctgtggccttgaattttacgccttagttccaggtccagggcagggccatccggattcaggatgcttcccagcccttcaggaatggcaggttttcatggtcctttctgagtgagttctgagtggtcatattggtgcccttggcagggagggctcctgactttcctatcttcacatcactgtccccaacccccaagagaggcctcttggcccagggactgcagggaggatgaagtcaggagcagaagcatggggtagggggctcaggtgggcagaggaggcccctctgtgaggaggaacggcaagcgaggagggaacaggggcaccggcagtgcctggcaagctgggtgatgtcacgactacgtcccgaccacacagtcctctcagccagcccgagaagcagggccctcccctgacccccatctgggcctgggcttcagttttctcctccctgcaatggggtgactgtttgcctccaggagaggggagcatgtaaaggtggccactctcttctggcagacatgccaggcctgggccagcctccacccctttgctcctgcagcccctgctgacctgctcctgtttgccacaccggcccctcctgggctgatcagggcccccctcctgcaggaagccctctgggacaagcccagcttgctgtaactgtggctttccactgtgacctgcaacgtgggaggctgttacttaaaactcccatgactggtggattgccggtccccagaacaaggccacgcatccctggaggccctcgagaccatttaaggtagttaaacatttttactttatgcattttcatgtgtatcagaaagaaaaaaaatgtatcatcagttcatcaaatccatgatttcttgaccaatattgctaagatgaggctgaaataggcatttccatttttaaaaaactgaatcactctgaagaaacagatggcaggcttccctggtggtccggtggttaacagtccatgcttccagtgctgggggcatgggttcgatccctgaaaattttaaaaaggaagaaaaagatggctcccccgtccctgggattctccaggcaagaacactggagtgggttgccatttccttctccagtgcatgaaagggaaaagggaaagtgaagtcgctcagtcgtgtgcgactcttagcaaccccatggactgcagcctaccagactcctccgtccatgggattttccaggcaagagtactggagtggggtgccattgccttctccaggcaaacggcctgctactgctactgctgctaaatcgcttcagtcgtgtccaactctgtgcgaccccatagacggcagcccaccaggctcccccgtccctgggattctccaggcaagaacactggagtggggtgccattgccttcagcctgctgctgctgctgctaagtcgcttcagtcgtgtccgactctgtgtgaccgcatagacggcagcccaccaggctcccccgtccctgggattctccaggcaagaacactggagtgggttgccatttccttctccaatgcatgaaagtgaaaagttaaagtgaaattgctcagtcgtgtccgactcttagtgacccaatggactgcagcctaccagggtcctccatccatgggattttccaggcaagagtactggagtggggtgccattcggcctagggagtgagaaatcacggctgtcttccctcttctcgccctctaggggtctctgtggagcctccctggagaggccgcggcggctccggggactggagggggagggggggttgagtcagccggtggccctcccctcgctgcccgtctcctccctttttaggcacaagctgggcgccctttttaggcgcagcctcaccctgcgggccactgcccgtgtttcggctccccggagataaaacagattgcctgcaccccgggtcatcacaaggattgtatgaccgtttcccagtgtgctcaccaccctccctctgattctcagagacgcgccctcgcctcaggaggctgctcatcccaggccaaggggcggcgtggggtccccagcgccccgcacagacactgccttctgaccacctcctcccaacagcttacctgccaagaaggcctcctgacccctcatcctgcccggtggtttggagaaagcctcatctggcccctccttctcggggcctcagtttccccctctgtgaactggcggattctgccaagctgacgtcctggccagccgcctccccgtggccagtgtcccccgggacacagctgaatgtccctgctcgggatgcaccttcccaagttggcctgtcaggaggcgggggcgagcagggaaacccgactcctctcagacggcccatcgcattggggacgctgaggcccggagcagcggcaccctcctggccagggtcattctcccgccccgccccgtccctccgggcctccgagaccgcagcccggcccgccccgggaaggaccggatccgcgggccgggccaccccccttccctggccgcgggcgcggggcgagtgcagaacaaaagcggggggcggggccggggcgggggcggggcggaggatataaggggcggcggccggcggcaccccagcaggccctgcacccccgggggggatggctcgggccgccggcctccgcggggcggcctcgcgcgcctttttgtttttggtgagggtgatgggggcggtcgcggggtactattttttcatttataattgggtattagctagcgagtggaaccacacccttattccactatagccaatttttgcgggggcatcttacattacagactcgcccgcctcttatttcggtacagcatatcagatcgtctctttactcagacactagtgattattgtctatagtacacaaaaagaacggttgtgtcggcgtaatggttgcattttccctcctcgtttctcctgaccacctcaattacaccaacactctactatttaaatcacgtattgtacgccaccctccgcccgcgaactaaaagaatgtgcagatattctgaagataaaatcgttcattgttacgccccgcgcgcttcgcgtatattactcttagaacttcttattcgcccgagcagttattcaccccccgcaactagatgtcgccttaatatttgttctaaccgttttggattctaacgataggcgggaaaggtagacattcgaccgctacgacaactaaaatcgacgagcacaggctatttatatcgcgaccacacgcgcgcggtatacanaccgtaaaattatctaacatcgagagtaagggcacagagcgaaatacaagcggcgtggtgggaggtgtgtctgtagtgaattcgcacctcgcgccgccgcctctgtgcgtcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngatataatattaataaacagcggatagatgtgtgtaagggaggaggtgcataagagattaaagagaggcgggcggagagaaatagagtagaggaggatgagagaaaaaagaaagcaagcgtaggtacaacggcgggtgggtagtatgataaagtgagtgtatatatttgagtaaaggaagggtagatggagtataaagaagtaaggagaggagagggcggcggagagagagagtgcaaagaaaataagtgggcaaaggcggggtgggtgagaagcagtagaagagaagatagagaagggggaaaaagaggaaaatgaggattagaacaagtaggacaggatagatgtgaaaaatgagatcaggtcaaggtggagaaaaagtagaaactggggcgtgattgtaaaaaagggaggccgcgatggggcagcaccataagcgaagagatgaattaatgaaagcaaggcagggagaatcaaatgagttgggtggaggaaggaggctgtgacttccttcgctgccggaaagagaactagaatagcctcgggctgtggggggaggtaaagataaagtgacttctgggccctgggggaggcccaggagtttctaccgagctgagctgggtgcctctcccaaatgcccaaccccctgagagtcgacgggagagcacagcctggccaaacctgggcagggcacacgtgtccttcaccccacagtggtcacgagcccagcgtggtccctgcgtctggcgggaaacacagaccctcacaccccacacaagggtccggccgctttcaaataacagcagccgtgccctctgggccggtgacccggacacagagagatgaagtccgcatctctcagagtgcgctgtcctccgcccggtcaggcccgggtcccctgcttctctgaggtcaccaggagggattgcatgtgggtctcagggacacaggttcagtgatgtgacagagggtagtgggtcccagcagggccggtctttggacccgtttttctgaaaagccagttggcgacctggggtcacagcaaagctgatcctgtttggccaggagtctcccagtgacggcctcccccagaacatcgggcccagtgggggctccagggggtagacttgcctcccagctcacgcccgtgtcttgacaagtccatgatttggtaaaattaatttgtgttggatggagttgatttagtggtgtgtgagtttctgtggcgcagcaaagtcaatcagttacgcatacacatgtatccagctcttcctacgattctgttcccatataggtcattatggggtgtcaggtagagcttcctgtgctacgcagtacggccttattcagttcagctcagtcgtgtccgactccttgtgaccccatggactgcagcacgccaggctcccctgtccatcaccaactcctggagcttattcaaactcatgtccatcgagccggtgatgccatccaaccatctcatcctctgtcgttccctctcctcctgccttcagtctttcccagcaccccctagagaagggaatggcaaaccacttcggtattcttgccctgagaaccccatgaacagtacggaaagtccttattagttttctattttatatatagcagtgcacacgtgtcagccccaatctcgcaatttatcacccccctccgccgccgattggtagtcatgtttgttttctacatctgcgactctatttctgttttgtaaacaagttcatttacaccacttttttagattctgcacatacgtggcaagcccacagcaaacatgctcaatggtgaaagactgaaagcatttcctctaagatcaaaaacaagacgaggatgtccactcactccgtttttactcaacacagccctgaacgtcctagccatggcaatcagagaagagaaagaaattaaggaatccaaattggaaaagaagaagtaaaactcactctttgcaaatgacatgacacttatacccagaaaatcctagagatgctaccagataactattagagctcatcagtgaatttgttgcaggatacaaaattaatacacagaaatctcctgcattcctatagactgacaacaaaagatctgagagagaaattaaggaaaccatcccacggcatgaaaaagagtaaaatacctaggaataaagctacctaaagaggcaaaagacctgtactcagaaaactataaaatactgacaaaggaaatcagacgacacagagagagagagataccacgctcttggatgagaagaatcgatagtgtgacaatgactatactacccagagaaacatacagattcagtacaacccctatcaaattcccaatggcatttttcacagaatcagaattagaacaaaaagttttacaagtttcagggaaacaagaaagatcctaaagagccagagcaatcttgagaaagaaaaatggagctggaagagtcaggctccctgagttctgactgtgtatacaaagctggcatgatttttaacagcaggggtgtaaatgaacttgttcacaaaacagatggtggggtgggcttccctggtggctcagctggtaaagaatcctcctgcaacgcaggagacctgggttcgatccctaggctgggaagatcccctggagaagggaaaggctacccactccagtattctggcctggaaaattccaaggaccatatagtccatgggtttgcaaagagtcggacacgactgagcgacttccaatcctggaaacgtcccattgtggacggtgaactggggttgtccaagctcagggtaaccgtttgctgagtgactgacactccttctcatgggttaaaatgtggggcccaaggccaggaccagaccccgcagtcagccaggcagaccctgtgcagccccagcgagtgtgtggccgccgtggagttcctggcccccatgggcctcgactggagcccctggagtgagcccattccctcccagcccgtgagaggctgggtgcagccctaaccatttcccacccagtgacagatccgcctgtgtggaaacctgctcttgtccccagggaacctggcaggactcagggagaatgtctcagggcggccacagatcaggggctgggggggcagggctgggtccagcagaggccctgtgcccactccccggaaagagcagctgatggtcagcatgacccaccagggcaccgacgcgtgcttgcacacaggccgccccctcatggtgacactcttttcctgtggccacatctcgccccctcaggtccctcctgctccccagctcctggcctgggaacctcttccccgccccggggacgtcagggctggtgtccactgagcatcccatgcccgggactgtgctgatcaccagcacctgcaccccctctcgggtctcaccaggatgggcaactcctgcccatccagcacccagcctcctgggtacacatcgggggaggagggagaagcctgggccagacccccagtgggctccctaaggaggacagaaaggctgccgtgggccagccgagagcagctctctgagagacgtgggaccccagaccacctgtgagccacccgcagtgtctctgctcacacgggccaccagcccagcactagtgtggacgagggtgagtgggtgaggcccaggtgcaccagggcaagtgggtgaggcccgagtggacagggtgagtgggtgaggcccaggtagaccagggcccatgtgggtgaggcccgggtggaccagagtgagcgggtgaggcccaggtggacagggcgagcgggtgaggcccaggtggacagggcgagcgggtgaggcccgggtggacagggcgagcgggtgaggcccgggtggacagggcgagcgggtgaggcccgggtggacagggcgagtgggtgaggcccgggtggaccagggcgagtgggtgaggcccgggtggacagggcgagtgggtgaggcccgggtggaccagggcgagtgggtgaggcccaggtggacagggtgagtgggtgaggcccaggtagaccagggcccagagcaaagccccggctcagcagtgatttcctgagcgcccactgcttgcagggacctcagcgatggtaaggcagccctgttgggggctcccgactggggacagcatgcagagagcgagtggtcccctggagaaacagccagggcatggccgggcgccctgccaggctgccccaggggccacagctgagccccgaggcggccaggggccgggacagccctgattctgggttgggggctgggggccagagtgccctctgtgcagctgggccggtgacagtggcgcctcgctccctgggggcccgggagggacggtcaggtggaaaatggacgtttgcgggtctctggggttgacagttgtcgccattggcactgggctgttggggcccagcagcctcaggccagcacccccggggctccccacgggccccgcaccctcaccccacgcagctggcctggcgaaaccaagaggccctgacgcccgaaatagccaggaaaccccgaccgaccgcccagccctggcagcaggtgcctccctctccccggggtggggggaggggttgctccagttctggaagcttccaccagcccagctggagaaaggcccacatcccagcacccaggccgcccaggcccctgtgtccaggcctggccgcctgagaccacgtccgtcagaagcggcatctcttatcccacgatcctgtgtctgggatcctggaggtcatggcccctctcggggccccaggagcccatctaagtgccaggctcagagctgaggctgccgcgggacacagaggagctggggctggcctagggcaccgcggtcacacttcccctgccgcccctcacttgggactctttgcggggagggactgagccaagtatggggatggggagaaaaatggggaccctcacgatcactgccctgggagccctggtgcgtctggagtaacaatgcggtgactcgaagcacagctgttccccacgaggcctcacagggtccttctccaggggacgggacctcagatggccagtcactcatccattccccacgaggcctcacagggtccttctccaggggacgggacctcagatggccagtcactcatccattccccatgaggtctcacagggtccttctccaggggacgggacctcagatggccagtcactcatccattccccacgaggcctcacagggtccttctccaggggacgggaccccagatgggccagtcactcatccatccgtctgtgcacccatccgtccaaccatcacccttccctccatccatctgaaagcttccctgaggcctccccggggacccagcctgcatgcggccctcagctgctcatcccaggccagtcaggcccggcacagtcaaggccaaagtcagacctggaaggtgcctgcttcaccacgggaggaggggggctgtggacacagggcgccccatgccctgcccagcctgccccccgtgctcggccgagatgctgagggcaacgggggggcaggaggtgggacagacaggccagcgtggggggccagctgccgcctggctgcgggtgagcagactgcccccctcaccccaggtacaggtctccctgatgtcccctgccctccctgcctccctgtccggctccaatcagagaggtcccggcattccagggctccgtggtcctcatgggaataaaaggtggggaacaagtacccggcacgctctcctgagcccacccccaaacacacacaaaaaaatccctccaccggtgggacttcaccagctcgttctcaggggagctgccagggggtcccccagccccaggaagccaggggccaggcctgcaagtccacagccataacaccatgtcagctgacacagagagacagtgtctggtggacaggtgcccccacctgcgagcctggagagtgtggccctcgcctgccccagccgcggtcagtcggctcagcaaccgctgtccactcccagcgccctggcctcccctgtgggcccaggtcaagtcctgggggtgaagctaagtcagggagcctcatccatgcccagcccggagcccacagcgccatcaagaaatgcttcttccctccatcaggaaacattagtgggaaagacaagagctggggggttctggggtcctgggggatcagatgaaggggtctgggagcagcagcagcctcaggcaccccaaaacaaggcccaggagctggactcccagggctgaggggcagagggaaggaaggcctcctggggggttggcatgagcaaaggcacccaggtgggggctgagcacccctcggctggcacacacaggcccccactgcagtaccttccccctcggagaccctgggctcccgtctcccgcctggcctgccatcctgctcaccacccagaaatccctgagtgcggtgccatgtgactgggccctgccctggggaggaaggagattcagacagacaggatgccagggcagagaggggcgagcagaggatgctgggagggggcccggggaggcctggggggcaggggggcaggagttctccagggtggacggcgctgtgctatgctcggtgagcacagaggccccgggtgtcccaggcctgggaacccagcagaggggcagggacggggctcaaaggacccaaaggccgagccctgaccagacctgtgggtccagaaggcagctgcgccctgaggccactgagtggccccgtgtcccgaaccaccgctgaaacatgggacacacgttcccaggcggagccactcctgccttccgggaggctcccagcgggctcatcgctccatcccacagggagggaaaccgaggcccagatgacgaacatcccggcgagcaggtcaaagccagcccctggggtcccctctcccggcctggggcctcccctctgcagggtgggaaaccgaggccacacaggggctccatggggctgccctctgccaggccctggacaccccgcgggtgacccccgcctctatcatcccagccctgccaggccctggacaccccgtggatgacccccgcctctatcatcccagccctgggggacagatgggaggcccaagcgtggaccccctggccaccccctaccccacagccgggaggagccgggagctggtggccaagggcctagaggagccagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaatatagagggggtgggataaagggtaatatgatgtttaggtagttagagttaaattagaagggtttggataaagattaataaaattacaagcgtacatatcgtgtgagtgtgggtgataatatttgtgtatgtggggaatagaagtgagtgtgagtagtattcaagatgtaagtgtgcgaatacaggtctgagcgatttgaatggaagtgaaaaaaagcgtgtgtgtggaggaggcgggagaggaagatagtgtgggggaagaaaagaaggctagtgggtaaagaaatatcagtaggcggttgacgaaagaagaactaggaagaattaatataaaaataaagggaggattaaaaaataaagagggaggaggtaacggaaatagttagttaagaaaagaatggagagtggaggtaagataaataagggagtaatgggagtgaggaggaataaataaaaaaatggtgagggaaaatagagtagaatgagaacaagaatgaaaaagggagtgaagggggtgaaaaaaagtgaagttgaaaaaagaggaaaaaaaaggagaagataaaaaaataaaataaaaaaaggaaaaaaaagaaaaaaagaaagaagggttaaaggacgaaaagaagggaagagaaaaaaaatagtttaagtgggggagggtaaaaaagaattaataaagtaaatatggttgtggtcgaaaaaaaaaaaaaaattgttgtgttgatgagaagaaaagaaaaaagaagaaagggaaaagcaaaaagaaaggagagaaaaagacaaccccaccgcccgggcgcatggagggtgaggatggcgcacgcccgcggatggcacagcatcacagcaatcctaaaacgttttcagaccggtgcatcttcaccgcgcgcgcgccccgcccggccctcctcccgccctgaccgcggacccccacccgcaccggggagcctacccccaccccggggacgctccgccacgctaaggtcaggactgccgtgaagacgcgccggggtgaaaacgttttatcttcatgacataagcgagtggttttgaaacaggtttacaaaccctcgtgaagacgcacccttagcgttaggttttgtttttttaccatgtgacgatgcaactattttcttcctctcttccacagtggctagtcgcctccagagcgaggggtatctcttgtacagagaccctcggaacatccggaggtagtttcccacctaggggtaaagcgagaaggctcattacgagggccggggctcctcggggaagggcagggccctggcgcagaggctctgccacctcagtgacacgcagaccacgcgcggcctgcaggcgccgggctctgaaagcaggcaaagcccgatctgctgacatcaggggttccgcagcagcgaaggtctggcccgcacctggcccactggcagggggtaagctctgcctcccgacgacagcaccaagttcaggaagggccacgcagacactggtgagacacggcccccccggagctgcccgagaagctctgactttgcactaaagatctctggcgcggtccaaaaatgtaaggcctctcttccttttatcttaagactttgatatttttacgatgtaataaataccaagaagggcttttaatttcagacagatgtaggataatttcccccgtagcccttgctgctttgtttagtaacgaaactcaaaccagaaataccaaaggaattttccaaagagtttcaaaagcgcttatcagcaatcactagactgctgcatacatcatcactgccccaaacaatagcctgcctgtgccagttactcaaagtactacttacttgacgaaaacaaatctagtcctaacgtttttacaaagaaactccactcttccgccaacttttcagaaacaaccactcgatcacgtggcaggggaccgtggctggactgggtgctggctccttctgtgaccaggcaacactgcccccttctcggcctccctacgcctcttgacaaatgttcatcagctgtaaagttcaccccacgagggacccacttctgctatttcccacgtacctaccccattataggagttttctttgtgacagtttctgcatttttcatggatttagaggtttacataatcagggctgctgaacagcatgagagacgtggccacaaggtccctcctgcaccttgccgcaggggcagggcgagttatctggcttgagcgtggttaccatcagggggtaaacacagtttccaggacgtttttgacaagacactgacccggatgcccccactaccaccgtgcaggtcctgcaggcctcccagcctcccaggcccttcccgaggtcccttcggaacttaggggactcggtctgcccccctgggttttccctgcaccagcttttgccccctctggacccaggtttcccaaatggaaaacgaaggtgtgggtatggaagctccctgggctcctctcagctgtgcctctgcatggtgatgacggctgcccatcggggggggcaggactggggcagctgcggacaccctcccaaggctgctacccccgagtggtgtggggcgctgtgggcacgctctgctcagcgcacctcctggaaaccagcgcctgccgtctgcccggggcaaccggcccgggagccaagcaccactgccgtcagaggagctgctggctgtgagtggacgccagtctagctctgaaccctgcccaggcctcctgaggtctgaacattgtaaaatcaggccccggacggcaactgcctctccctcctgccgtctggtctccataaactgcatctcaggacaaatcttctcactcaccagggctgaaacagaagactgcagctatctttctcaaatctaaggtgtgctacagggcaagtcgcagaaactgtctggcctaagcatctcatcagatgcctgagacaagagctgtggacgccaagctggagccagagctcctcgcgttctgcccacctggcaccgcgttccacccagtaaacgcaggcttgattttcaaaagtaccaccgactcagagccaatgctaaaccgaccacttttcctgcccattagattgggtgaaggtttctttaatcaatctgccagtcaccacatgccgcctctgtgcccacaggctggcgaagacctttctgagctacggcatgtggcaggcagcggcacctctcttcagtacggccagctgtcaaggggagcgtttctgtgatgatgtgaaaatacattgcatccggccccgtgtttcatgaacacgggtgaggaaaggaaacacacaaagttctgatgcgactgacagcacgggtctcataactcaatacaagtcagacaaaccacagggagtcacagggaatcccaatagcctcatctagtgtgaccatcatgaggcttaatttattcagtgtattcaatcataaagagggggaaaaattgtaaaaaaaaaaaaaaagaaagagtgaaatgtgtaatactgaaaactgttgctaggagaagcaagcattggcgtttgtaactgctttgactccccaagacccacactcgcctcgctacaaaagggaggcactgctgctcagtacttgcacacccgaactgcggatttgtaatttaaaaatgtgtgtgtggacacagcacaagccagagactgccaaaggttgagggacactggaagaacttaatatacttggtgcatgctgccagtgacagtcagtcaccagctgattcaatagagtgccgaaaggtcaccttttaggtaaggatgaaggggttctgggctcgtttacttgcactaactcagagttagtccgagatatccgaagtgccaggtgcctcccatttgctgatggatctagctcagggacggctgggccctagccatccaaaaatcaagcattgttctcccaacctgtcttctcgctgataatggaaggtcagaacgcccacccgcccacctcaaagtcaaagaacaccaagcgggtgagtccccactaagctcggtgtttccaatcagcggtttcaggattccagctggggcaatgagggagggagcgtgcgagggatccaacacctcgccccgtgcgcagcaagggataacccaacaccccgtttctgtacgtccggctggagttgtggaactcagcgcggacccggggccaccgcgacccccgggaccctggccgcgcggcgcatccccgctgccgggacacgggtaagcgtccccaaactgccggacgcggggcggggccttctccgccacgccccgataggccacgcccaaggacaaggatggtcgtgcccagacggccggggcgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncggaggggggggggcggggcgggggctgccgccgcgcgtataggacggtggtcgcccggcctggggtccggccgggaatgaccccgcctctccccgcatcccgcagccgccccgccgcgccctctgccgcgcacccgcctgcgcacccgccgccctcggccgcggccccggcccccgccccgtcgggccagcccggcctgatggcgcagatggcgaccaccgccgccggagtggccgtgggctcggctgtgggccacgtcgtgggcagcgctctgaccggagccttcagtggggggagctcagagcccgcccagcctgcggcccagcaggtgagcaagggctcaggggaaactgaggcccgacacagagccgcagcaagaaggatcctactggtcactcggctgttggcctggggtcatcacaggcgggctctcccaacccatcccctgaggccaaggtccctagaaccccgtgggcagacaccaaccagccctttaaatatggggaaaccaaggtgcttaggggtcagagatagccctaggtcgcccaaccctagtagaagggagggctgttggagttcctgagtgcccgctctcccaccccccgggaggccccttcctgagcccaagggtgactggtagtcagtgactttgggcctgccgacctgtaccccactgggcaccccaccagtcctgagccacatttgggcttagtgacggggtcagggatcatgaggatcaatgtggctgagccaggaaggtgttagaacctgtcggcctggagttcataccagcactgccctgggcttttctagacccatgtcccgcctcctgccccacctgcccctgttcccgcaccccaccagcagcggcaggggcttcgagagggctgtgggctcaccctatttcagggatggagccgctaagacctggggcacactgcccgctagggacccctgaggcaccagggccgggggctctgcggaggggcagccgccacccccagctttggagtcctctcccgggtgcccagcccgagctgatccggctgcctcccacgctgtgccccagggcccggagcgcgccgccccgcagcccctgcagatggggccctgtgcctatgagatcaggcagttcctggactgctccaccacccagagcgacctgaccctgtgtgagggcttcagcgaggccctgaagcagtgcaagtacaaccacggtgagcggctgctgcccgactggcgccagggtgggaagggcggtccacggctcccactccttcggggtgctcccgctattcccaggtgctcctgcacttcccatgtgctcccgattctccctggtgctccctctcctcctggctgctcctttgcctcccaggtgctcccacttctccctggtgctcctgctcctcccggcggctcctgtaccttcggcctgacctcctccctctacaggtctgagctccctgccctaagagaccagagcagattgggtggccagccctgcacccacctgcacccccctcccaccgacagccggaccatgacgtcagattgtacccaccgagctgggacccagagtgaggagggggtccctcaccccacagatgacctgagatgaaaacgtgcaattaaaagcctttattttagccgaacctgctgtgtctcctcttgttggactgtctgcggggggcgggggggagggagatggaagtcccactgcggggtggggtgccaccccttcagctgctgccccctgtggggagggtgaccttgtcatcctgcgtaatccgacgggcagcgcagaccggatggtgaggcactaactgctgacctcaagcctcaagggcgtccgactccggccagctggagaccctggaggagcgtgccgcctccttctcgtctctgggggcccctcggtggcctcacgctctgtcggtcaccttgcccctcttgctgatgcaatttccccgtaattgcagattcagcaggaggaatgcttcgggcctttgcacctgaccgcatgagcagaggtcacggccagcccccttggatctcagtccagctcggccgcttggccgtgacgttccaggtcacagggcctgccggcacagaggagcaggcccttcagtgccgtcgagcactcggagctgctgcctccgctgagttcactcagtgtctacgcacagagcgcccactgtgtaccaggccctattccacgttccccagtcaccgagcccccagggctggtggggacctgccctcgggtacactgtgtcccgtcacgtggctttacgtgtgtctctgagggaggctggcattgcggtccacctctcagcacaaacatctgtcccctgggaagggggtcccatttctgggtgcgagcagccccctggggtccgtgtctcctccttacctggctcaaggccccggctcctgggtcctggacagcagggagcccacccctcggggctgtggagggggaccttgcttctggaggccacgccgagggcccaggcgccgcctccggccgtcgccctgagggagcaggcccgacgccagcgcggctcctctgtgaggcccgggaaaccctgcctgagggtgcgggtgggcaggtgcccctgcccccaggctctcctgtgtgagtgacactcaccagccagctctggatgccacccatccgggttctccaggaggcactcatagcgggtggggtcccctccctcccccctctgtggagggagggagtctgatcactgggaggctggtggtccgtacccgcccccccgactctggacgtgtttactacccccgcctgggctcaggacagggcattggatgggaaggacagggctgggtcctggccaggctgggggctctgcagggcatgggtgcccctgtctcttcttatattccaacgtcactgcaggggggcgcaaatcttggaccccacttactgatgatctgcatcaggacataggtcccccctcctgcagcggggggctggccacggagggcgctggggaaggcccctcctccagcccctcggcgaggctcaccaggtgcccatcctcagccagcagggcgacgctcgctgggagggcggagagggaggcagggcagggctggtacgacccccgctggggcgggggggccctcagccggtcctccagcacccttgctgccccccctcaccgtcagggggcacctggccgctctgcctcaggtgggcggtgagggtcccaaggccacaccaggtgttcaccagctcccagcagctggctgtgggagaggggcagaggtgggcgcatggcacccgccttccccccagaccaggatgctctgccttcctcccgcccatctccccagacatctgaaggactcttgcctccaccatgcagccccgcctccaccagaagctcaggttccccgccccccctccccgaagctgcaggacccctgaccagcgaagagatgggacagttggaacacacgctcccccagcagcggcacagcagctgtgtggcccagaagagcccgcctgtttccctcaagcaactccccatggatgtcatcccatggacacccccttccccacaccgcctcctcgttctccccctccaaggcagagggaacgcacccccacctgtctgctaggacaggggaccccacttacctccgaacatcaccttgataaacatggccgtggtggggacagatccctccgacccccaacttccgacctggggaaggagctggggtggagctcgactgcagggtggggccctgtgggaggtgtacgggtggagagggtgatgggtgggtgggctcaagcggagctccttgctcagtccaggcggtccctgcagctagtccaggatcctcagccttctccccctcactggatcagggaagactgaggttccctcccctgcccccccacccagcttccaagctggtctctgtggcagtgggagctgccaagaggtctgagcggccagtatccgggtaacggggtttgtggagggtccgggcattcccggtgcagggctctagtgggggctggagcctcgggcccagagctgtccagagaccagtgccctcccaccgccgccgcccgcaaggagagacagagctcccaggcggggagtcggaggttcctggagggggagcatcctcaactctgcaggcccccttcccaggcgcactcccggcctccccgtcttctgtcccctgctcttgttgaagtatgattggcatacagttcacagccactcttcggagtgttctccacactaaggatacagaacatgtccctcgtccccccaaactcccagccaggctgtcacgaagagggaggcggccgacggggcagggccttgcactcctgcgtgtggggtccacaggggtcgtccccgtgtcggtggccccttcctctcacgccaggagggtccccttgcctggaggtgccgtggatccgctcgctgcctgctctttgggttgtttcccgcatggggtgatgatgaagaggccagtacagacactcgccagcaggtctctgggtgaacaggcatttatttctctttcctgagggcagatcctgggagtggggtgccggaccgtccggggagagtatgcttctgtttctaagaagctgccgtgttctccagtgtgctgcaccatgtcacggcccctctgtgcgtctggactcaggagacctccttctcagcggccctcccccccaggtggtcaggccatctgtgcccttctgggggcagagctcagcgccggaggcgggaggaggcccagatcccagcgcagcccaccagcgttgctctgcttccctcggcattcatagctggagaaagggcaaggagcaccggctgaagccccacctggaggacgcacttcgatggcagcaggtgctcagaggtggccccgggcagcattccccagacgcacaggccagtgctttcttcccaggacaccactgtgtctggggacccgagtcctgcagcacggtcgggagcggctgtgcccagattccggcctgcacccttggctccagccaccacccctgtttgtcaaggggtttttgtctttcgagccgccgaggagggagtcttttgtctgcagtgtcacagaagtgccataaagaggggcccacagtgggagctttataacattggtgcggagggctgtaacaggtcagggaggcacttgagggagccttctagggcgatggagatgttctaaaatttggtctgggtacaggctacagagatgtgtgggtgtgtgtgtgtgtgtgtgtaaaaccctcgagccacacgtgtgaggtctgtgcatgtgaccgtacacaggagacctcggtggaaagcagccacctgctctgactgcacctgtggatttccagctcctgccctcaggcggccctgcggggcccactggctgacggggagacggcaccgccctcccccgctgtcagggtgggggggctgacgatttgcatgtcgtgtcagggtccagcggcctcccttgcgtggaggtcccgaagcacctggagcgccgcccgcagaacagcggactcctgcctgcctccctgcctctggccatggcctgcccgcctctggccctctttctgctcggggccctcctggcaggtgagccctcccaaggcctggctcacctaggggtgtgtaagacagcacggggctctagaagtaaatcgcggggaagtaaatcgtagtgggcaggggggatggtttccgaaggggccctgagggggacaggagacctggcctcagtttccccactggtgagtgaccagatagccagggtacctttggactctgactctggggggctctcagagactggtctcctactcagtttttcagaggggaagctggtgtggccttgtcactgccctgcagggcctcagggacaagctatccctgaggaggtctccagcagtcagtggccggaggctgagccgatggatatagtaacagcccaggcggcctcttgggggtggtcagcctgtagccaggttttggacgagccgaagtgacctaagtgatgggggtctgcagagcaagggatgagggtgggcagcaggaggacccagagcccaccagcccaccctctgaattctggacccttagctgcatgtggctccttgggaagacggggcttaagggttgcccgctctgtggcccacacagtgctgattccacagcactggctgtgagcttttgggagcagattctcccggggagtctgacccaggctttgtggggcaggggctggagggaaggggcccaggccagacctgagtgtgtgtctctcagcctcccagccagccctgaccaagccagaagcactgctggtcttcccaggacaagtggcccaactgtcctgcacgatcagcccccattacgccatcgtcggggacctcggcgtgtcctggtatcagcagcgagcaggcagcgccccccgcctgctcctctactaccgctcagaggagcaccaacaccgggcccccggcattccggaccgcttctctgcagctgcggatgcagcccacaacacctgcatcctgaccatcagccccgtgcagcccgaagatgacgccgattattactgctttgtgggtgacttattctaggggtgtgggatgagtgtcttccgtctgcctgccacttctactcctgaccttgggaccctctctctgagcctcagttttcctcctctgtgaaatgggttaataacactcaccatgtcaacaataactgctctgagggttatgagatccctgtggctcggggtgtgggggtagggatggtcctggggattactgcagaagaggaagcacctgagacccttggcgtggggcccagcctccccaccagcccccaggggcccagactggtggctcttgccttcctgtgacgggaggagctggagtgagagaaaaaggaaccagcctttgctggtcccggctctgcatggctggttgggttccaacactcaacgaggggactggaccgggtcttcgggagcccctgcctactcctgggtggggcaagggggcaggtgtgagtgtgtgtgtggggtgcagacactcagaggcacctgaaggcaggtgggcagagggcaggggaggcatgggcagcagccctcctggggtagagaggcaggcttgccaccagaagcagaacttagccctgggaggggggtgggggggttgaagaacacagctctcttctctcccggttcctctaagaggcgccacatgaacagggggactacccatcagatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagagggtgggtgggtggaatttaatatagtggtgcgcgtggagcgtgggcggcgcatttaaggcggtcatctaaaatagtggatagggggtggtgtgacaataacgggtggtggatgtggtttacggggggtgcaatagttctgagtttgttagtgtcttcttgatggggttgcggcgtgtggacctacgccttgagtatgtggggggggaaaagcagtgagggtagtagggatgggaaatattggtggaggttctttgttggtgtattttttggtattatgttgggtggtggagtggtgggttgggtgtaatttcgcttgcgttatgtgttttttttctttttcgtgtcgtgggttgggttggttggtgctttgtggtggtggtgggttgtggtataaaaaaaaatgtgtggttgtgctcagcttagccctataacggtcggctttgtttcttgtttgttctgtgggcgtgagcggatggctcgggcctccgtgctccgcggcgcggcctcgcgcgccctcctgctcccgctgctgctgctgctgctgctcccgccgccgccgctgctgctggcccgggccccgcggccgccggtgagtgcccgccgtcctccagcccccccgccccgccccgccctccacgccgaggggcgccggctcgcagagctggatccaagggggtgcccgggagtggcccggcgcggcccgttaccccgaaacgctgtctgggtgccccgggggtgtggtggatagtgagcttcccgtccctggaagtatgcaagtgaagccggcgccgggatcgctcgggctggctggtgagcgggcgggactcggtcgggcgctagacgcacgccgccagccccccagctcccagacctgcccactccgcgcccgcccggccgcgatcccgggtgtgtgtgtgtgttgcaggggagggacagcgggagtggctacagggctcccgactcaccgcagggacaaagacccgcgggtccccagctggcgtcagccgccaggtgtgtggcctcggtgagcacacctccaggcgggagggttgagggaagcgctgtggggagggcatgcggggtctgagcctggaagagacggatgctaccgcctgggacctgtgagtggcgggattgggaggctatggaatcaggaggcagcctaagcgtgagagctccggtgtggcctggcgggggtggtaggggggggacgcccctgtgtgtgccagcctgcgtgtgccctaaaggctgcgccctcccccactgctggggcttcgggggaccagtcacagcctaggctactgcaggcgcacagctccccgggagcccggcccacgcgggtgtgccgctgagcctccagcctgtcggggcaggggtggggggcagggatggggtcgttagcggggttgggggcagacgcccaggcagactctctgggcacagctccggtgacaagggaggtctggcaagcctgggccccttctgtccagccacgccagctctgccctggccagtcttgccccctggcagtgctggggatggaagggggagcgggtacctcagtctgggggccctgcctcctccccagccccgcccggccccctaggcctaggggcagagtctaggggtcaccctggggagctgctgaatccgcgggtttaggaaccggagggacctgggcttttgaaccacgtggccctaggtgagccctccggcgcctcggtagccctcacccccagccttgtccaggtgggcgggtgggaggcgacagtgcccactgctgggctgaacagcgtctgcagggaggccaggagagctgggcacacggacacgttccatcacctggagctgccactgtgccacttgtgcggggtcaggcggggtctgagccgggctgtcatctgtcacgccacagatatgcagggggcactcggggtcgcctcggacatgcttatccctggacggctgttggcagggccgggaaggctctgtaaatatttatccatcccagctcacagctttcagggttgatgaaagccccgccgcccgcccactgtgggggaccccgccttcccttctggagccagcggggtgagggggtgggggagatggacctgcctgcccaggagcaggcggtgtgactctggcaggtcacttgacctctctgagcctcagggagggcccgggatggtgtgcggatgctctctgccttcctcccagcctgaccagtgtcctcccctcggggtcgcctcctgcccaccgcagagggggtggctatggggacctgggccgatggcaggcaggccggagagggcatgcccggctcagccgtgcccagcacttcccagtccaggggcccccgccactcccagccgctggctgcctcccattttcccgattgcaggttggccccgaggctgaccggagcctctggctcagctgggagactgaattccccaagcaattcctcaaggatgtgtgaggctgtggtgtggtgcctatccgggagaggtggggtgagcggactgggcacctccgcccagggcaggcccagggagacgctggctgacgagcaggcaggcctgcaaggaggacgagcagccatctcaggaatgtgggttttggagacaagccacagctgggggggtgggggggccatgggtggggaggcctgatccccaggtctaggtccagctctgggctccctcgccgtgtgaccctgggccaagacctggacctctctgggccccgtctcttcccctgggaggtggggcgatgcctgctccccaatcccccagggctgtggatgaggcagacgaggtgtgtgctcatccccacctcactgccttccagcagccccgggcggggggggtggtggggactggcgcacccaggtgaggatcaggccttggagctagggagggccccccagccccaggccagaaaggacacggggagacagaatgcaggagggcggcagagcaggggccagcggtggggaaactgaggccaagagcctgtggacgatgtgctccaggaaaggacctcgctgcctggggcctggatcctagagcctccaggagcggtgaccatgacgtgggcagggaaccggaggccccggcttgcaggtggacccggcgcgagtcactcttcctctctggccctgagagcttccttccagctgccgctcctgtgttctaatgtcaagtctggaggcctggggggcaggtgggggctgactgccaggtgggggagggcaggaatttggcagagcagcgtcccagagtgggagaagccagcccatggaggggactctctccatgcctgctgccccaaagggcgttatagagagaggtcggttaccccttcgccatggccccgttcccattgaacagatgggaaagtggaggctgagagaaggctgtgacttgcccagggtctccgtggcatggaactgggcctgctgagtctcaggccggggatctcgctgctgcactgagcacgccaggatgcaggggtctgggcctggacctagcgcctcgtgggggcaagagaggaaggcacgctgggcctgcctgtcaccctccaccccaccgtggcttgttgctcaggccttcctgggggcagaggagaggggagatttcactcgctggcaggctaggccctgggctctctggggctccgggggaacaatgcagccctggtctttctgaggagggtccttggacctccaccagggttgaggaaaggatttctgttcctcctggaggtcacggagccgacatggggaggagcaggggcaggcccggggcccacatcctcagtgtgagacctggacgtgtgtcctcccacctgacgctgggggtggggggtgggggccgggggggatccagtgaaccctgcccccaaattgtctggaagacagcgggtacttggtcatttccccttcctcctcttcgtttgccctggtggggacagtccctcccctggggaagggggaccccagcctgaagaacagagcagagctggggtcaggggtgtgctgggagcgcagagagcctcctgctctgcctgctggtcattcctggtggctctggagtcggcagctggtggggagcggctggggtgctcgtctgagctctggggtgcccagggcctgggagagttgccagaggctgaggccgagggtggggccctggcggcccggctcctgccccaaatatggctcgggaaggccacagcggcactgagcagacaggccgggccagacgggcgctgaggctcccggcctctcccccagctccgctgtgaccctcacctgcggcccggggtgccagggcccccgcttggttctgccgtgtctttgcaggctgatcccacgggctctccctgcctctctgagcttccgccttttccaggcaggggaaccgcgacctccaggctgggacgcggggagggtgtatgcgccaggtcagaatcacccctccaccgggagagcgtggtccaggggccctggcagggtggggaccgagcatctgggaactgccagccacccccacccatgcagaggggacatacagaccacacggaggctgtgcctccgctgcagcaactggagaacacccagccgcggccaaacataaataactaaataataaaagttttaaagatcgttacttaaaaaaacaagtgtgccccagtgatcggaccccagttcccggtgccctgagtggtgccggccctgtgctgagcatggcctggttggttcacccccagatccacactaaagggtgggatcacccctactagtcaggtgagcagatgcagggggggagggcggcagcccctccatgctggtgggtggccgtggtgggtgtcctgggcaggagccagctcacggagctggagaggacagacctggggggttgggggcgcccaggaagaaacgcagggggagaggtgtctgccgggggtgggggtcccttcgaggctgtgcgtgaagagggcaggcgggcctgcagccccacctacccgtccccggcccaaacggcgggagtaagtgaccctgggcacctggggccctccaggagggggcgggaggccttgggatcagcatctggacgccagtcagcccgcgccagagcgccatgctccccgacggcctccgctggagtgaggctgcgctgacacccacaccgctgacccgggcctctctcccgctcaggatgccccccgccgccaccccgtgagcagagggccacagccctggcccgacgcccctcccgacagtgacgcccccgccctggccacccaggaggccctcccgcttgctggccgccccagacctccccgctgcggcgtgcctgacctgcccgatgggccgagtgcccgcaaccgacagaagcggttcgtgctgtcgggcgggcgctgggagaagacggacctcacctacaggtagggccagtggccacgagctggcctttgatctccacctgctgtctgagacacgctggagctggggggagggcagatccctatggccaacaggctggagtgtcccccaactcccgtgcccactgctcaacaccccaaacccacacttagatgcactcccatgccctcccttgggagcacggtctccacacccacctggccaccccacacacccgtggggcacggccgttagtcacccacgcaacctctgcgggcaccgtgctgcgggccaggccctgggactctcagtgagggaggcagacacggcccctcctccgggggagcgaggtgctccccacgcccggttcagctctagcaccgcactcgggaccctcacagggagggacccactggggcaggccaggtgacggctcgggtgacctcggcccctggcgctgagactacacttcctgcagtgggcggcgaagatgggtgtggtgtcccacgtcgttgcagcggggactcctggggcctcggaagtgtcctgggcggggagcctggggagcaggaagggcaggtcttggggtccaaggcctccccacggtcaggtctgggagggggcctcggggctcttgggtcctttccgcccagtgcagaccctcgcggccacctaagggcacacagaccacacaaagctgtgcccatgcagtgtggggagtggtgcgcaccctcagagcacactgggcccacatcacgcacgcctgccccctcactgtgcatccggggaaactcctggccccgacagccagcggggctgacgctaccccgtgagccagacccaggcccccctcaccgcccctgtcctccccaggatcctccggttcccatggcagctgctgcgggaacaggtgcggcagacggtggcggaggccctccaggtgtggagcgatgtcacaccgctcaccttcaccgaggtgcacgagggccgcgccgacatcgtgatcgacttcaccaggtgagcgggggcctgagggcacccccaccctgggaaggaaacccatctgccggcagccactgactctgcccctacccaccccccgacaggtactggcacggggacaatctgccctttgatggacctgggggcatcctggcccacgccttcttccccaagacccaccgagaaggggatgtccacttcgactatgatgagacctggaccatcggggacaaccagggtaggggctggggccccactttccggaggggccctgtcgaggccccggagccgggcccgggctctgcgtccgctggggagctcgcgcattgccgggctgtctccctcttccaggcacggatctcctgcaggtggcggcacacgagtttggccacgtgctcgggctgcagcacacgacagctgcgaaggccctgatgtcccccttctacaccttccgctacccactgagcctcagcccagacgaccgcaggggcatccagcagctgtacggccggcctcagctagctcccacgtccaggcctccggacctgggccctggcaccggggcggacaccaacgagatcgcgccgctggaggtgaggccctgctccccctgcccacggctgcctctgcagctccaacatgggctcctcctaacccttcgctctcaccccagccggacgccccaccggatgcctgccaggtctcctttgacgcagccgccaccatccgtggcgagctcttcttcttcaaggcaggctttgtgtggcggctgcgcgggggccggctgcagcctggctaccctgcgctggcctctcgccactggcaggggctgcccagccctgtggatgcagccttcgaggacgcccagggccacatctggttcttccaaggtgagtgggagccgggtcacactcaggagactgcagggagccaggaacgtcatggccaagggtagggacagacagacgtgatgagcagatggacagacggagggggtcccggagttttggggcccaggaagagcgtgactcactcctctgggcacagctgggaggcttcctggaggaggcggttctcgaagcgggagtaggataaaaggtattgcaccccatgaagcacgtgtgatccttgcccctagagacaaggctctggggctcagaggtggtgaagtgacccacatgagggcacagcttggagaatgtcgggagggatgtgagctcagtgtgccagagatgggagcctggagcatgccaaggggcagggcctgctgcctgagagctggcactggggtgggcagccaagtgcagggatggagcgggcgcccaggtggcctctttgctgctcagaacgacctttcccatgtatacctcccagcgccgctggcattgcccagtgtccttcttgggggcaggagtaccaagcaggcattattactggccttttgtgttttatggacaacgaaactgaggctgggaaggtccgaggtggtgttggtggcggaaggtggccgctgggcagccctgttgcagcacacaccccccacccaccgtttctccaacaggagctcagtactgggtgtatgacggtgagaagccggtcctgggccccgcgcccctctccgagctgggcctgcaggggtccccgatccatgccgccctggtgtggggctccgagaagaacaagatctacttcttccgaagtggggactactggcgcttccagcccagcgcccgccgcgtggacagccctgtgccgcgccgggtcaccgactggcgaggggtgccctcggagatcgacgcggccttccaggatgctgaaggtgtgcagggggcaggccctctgcccagccccctcccattccgcccctcctcctgccaaggactgtgctaactccctgtgctccatctttgtggctgtgggcaccaggcacggcatggagactgaggcccgtgcccaggtcccttggatgtggctagtgaaatcagtccgaggctccagcctctgtcaggctgggtggcagctcagaccagaccctgagggcaggcagaagggctcgcccaagggtagaaagaccctggggcttccttggtggctcagacagtaaagcgtctgcctgcaatgcgggagacctggattcgatccctgggtcagggagatcccctggagaaggaaatggcaatgccctccggtactgttgcctggaaaattccatggacagagcagcctggaagctccatggggtcgcgaagagtcagacacaatggagcgacttcactgtcttaagggccacctgaggtcctcaggtttcaaggaacccagcagtggccaaggcctgtgcccatccctctgtccacttaccaggccctgaccctcctgtctcctcaggcttcgcctacttcctgcgtggccgcctctactggaagtttgaccccgtgaaggtgaaagccctggagggcttcccccggctcgtgggccccgacttcttcagctgtactgaggctgccaacactttccgctgatcaccgcctggctgtcctcaggccctgacacctccacacaggagaccgtggccgtgcctgtggctgtaggtaccaggcagggcacggagtcgcggctgctatgggggcaaggcagggcgctgccaccaggactgcagggagggccacgcgggtcgtggccactgccagcgactgtctgagactgggcaggggggctctggcatggaggctgagggtggtcttgggctggctccacgcagcctgtgcaggtcacatggaacccagctgcccatggtctccatccacacccctcagggtcgggcctcagcagggctgggggagctggagccctcaccgtcctcgctgtggggtcccatagggggctggcacgtgggtgtcagggtcctgcgcctcctgcctcccacaggggttggctctgcgtaggtgctgccttccagtttggtggttctggagacctattccccaagatcctggccaaaaggccaggtcagctggtgggggtgcttcctgccagagaccctgcaccctgggggccccagcatacctcagtcctatcacgggtcagatcctccaaagccatgtaaatgtgtacagtgtgtataaagctgttttgtttttcattttttaaccgactgtcattaaacacggtcgttttctacctgcctgctggggtgtctctgtgagtgcaaggccagtatagggtggaactggaccagggagttgggaggcttggctggggacccgctcagtcccctggtcctcagggctgggtgttggttcagggctccccctgctccatctcatcctgcttgaatgcctacagtggcttcacagtctgctccccatctccccagcggcctctcagaccgtcgtccaccaagtgctgctcacgttttccgatccagccactgtcaggacacagaaccgaactcaaggttactgtggctgactcctcactctctggggtctacttgcctgccaccctcagagagccaaggatccgcctgtgatgcaggagtgagtgaagtcgctcagccgagtccgactctttgcaaccccataggactgtagcctaccaggctcctctgtctatgggatttttcaggcaagagtgctggagtgggttgccatttccttctccaggggatcttcccaaccctggtctcccgcatagcaggcagactctttactgtctgagccaccaggcaatgcaggagacctaggttcagtctctgggtggggaagatcccctggagaagggaatgacaacctgcttcagtattcttgattggggaatcccatggacaaaggagcctggaggcctacagcccatagggtgcaaagagacacgactgagcaagtcacacacacagagccctacgtggatgctcatagcggcacctcatagctgccatgtatcaggtgttggcatgggcagccatcagcagggggccatttctgacccactgccttgttccaccggatacacgggtgccttcctgtgtgtcgggcccactcggctgtcagcgcccaagggcagggctgtcgggaggcacagggcacagagttaaggaggggatggggacgttagctcctccccagctctcagcggatgcagcaggcaaaacaaacgctaggaatcctgccaaacccggtagtctctgcccatgctcgccccatccccagagccacaagaacgggagctggggggtggcccggagctgggatactggtccctgggcccgcccatgtgctcggccgcacagcgtcctccgggcggggaaactgaggcacgggcgcctccggcttcctccccgccttccgggcctcgcctcgttcctcctcaccagggcagtattccagccccggctgtgagacggagaagggcgccgttcgagtcagggccgcggctgttatttctgccggtgagcggccttccctggtacctccacttgagaggcggccgggaaggccgagaaacgggccgaggctcctttaaggggcccgtgggggcgcgcccggcccttttgtccgggtggcggcggcggcgacgcgcgcgtcagcgtcaacgcccgcgcctgcgcactgagggcggcctgcttgtcgtctgcggcggcggcggcggcggcggcggaggaggcgaaccccatctggcttggcaagagactgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgcaggtgccggcggtgacgcggacgtacaccgcggcctgcgtcctcaccaccgccgccgtggtaaccgcccccgggggttgccaaggttacgattggaccctccccgccccgaccctgctcccctagggtgggtgggtcggggggcagtttctaagatctcctggttccgcagcagctggaactcctcagtcccttccagctctacttcaacccgcacctcgtgttccggaagttccaggtgaggccgccccgccccttgcacttgctggcccaacccctcccgcccagcgctggcctgaccgccccccaccccgcccaccccacgcaggtttggaggctcatcaccaacttcctcttcttcgggcccctgggattcagcttcttcttcaacatgctcttcgtgtatcctgcgccgtggtggaagcgggaggagggcggggcgggggaccgggcgggaggcagcgggccccgggaagctgagaccctccaaggggcacgcttcctataccaaagccgcaggttccgctactgccgcatgctggaggagggctccttccgcggccgcacggccgacttcgtcttcatgtttctcttcgggggcgtcctgatgactgtatccttcccgggctcggggacctatgggtccgggcctctgctggccctgaggccctgcttgagcgcatgccacagagggagagttgcgaccccgagctgagggtgtttttgagcgtacatcacgtgctcagctgcaggtgcccctgtcgaactccagggctacacccaaaataccacagggcagggtgcccaggggctgagtcctgaatgcaggtagccaggaggatctagggctgggcccgggggctggggtgaagtggagaggcagggccgatcagggggcccctggaggccaccgtttggtcttagagtgggaagcgaaaccaacctgcttgagggtttcaggggtttaggaagtcagaggggccctgggcagggcacaagaccttgactctggcccagctactggggctcctgggtagcctcttcttcctgggccaggccctcacggccatgctggtgtacgtgtggagccgccgcagccctggggtgagggtcaacttctttggcctcctcaccttccaggcgccgttcctgccctgggcgctcatgggcttttcaatgctgctgggcaactccatcctggtggacctgctgggtgagcctgctgtccagggagcctgccccaagctgggtgtgctgggccagagccctggtcctctccccgcccccacccctcttccccactcctggcgcccccatccttccagcccctccaacaagtcagcctataggttttacttattcgagcctgacccatttgctgacgcttgtgtggggcccgacccggtagggatgggtggctcagggtgcctgctcacagctccacttcttctgacgtcctcaggcctgacctcctcccaggttctgcctactctgggccaagcctggccccacgctgggctggctggccgtgcagggcatcagacccccatgctttgggggcttcagggctgtggagggtggcctcggcattggcgcctctcccacagggattgcggtgggccacgtctactacttcctggaggacgtcttccccaaccagcctggaggcaagaggctgctgctgacccccagcttcctgtgagtgctgacagccttccccacccccttccccagatggctctctaccccatgagggggggggaccctgccagctgccgctcagcgtgggctcctccccacaggaaactgctactggatgccccagaggaggaccccaattacctgcccctccccgaggagcagccaggacccctgcagcagtgaggacgacctcacccagagccgggtcccccacccccacccctggcctgcaacgcagctccctgtcctggaggccgggcctgggcccagggcccccgccctgaataaacaagtgacctgcagcctgttcgccacagcactggctctcctgccgcggccagcctctccacgcggggcaggtgctgctggccgagagccagggccaccaagcctgacgtgctctccgacccagaacattggcacagctggaggcccagagagggtccagaacctgcccactcgccagcagaactctgagcacagagggcagccctgctggggttctcatccctgccctgcctgtgccgtaattcagcttccactgatggggctcacatctcaggggcggggctgggactgggatgctgggttgtgctgagctttggccgtgggggccctcctgtcccgaactagcaacccccaaggggacctctgcttcatttcccagccaggccactgaaggacgggccaggtgcagaagagggccaggccctttctgtgactccgaagcctcaagtgtcagtgtttgcagagtccagtggctgaggcagaggcctctgggaagctctgcccctgccgtttgcagctgaggccggcaggagcctcacctggtccccagctcacgggcattggaggaccagtccgcacggtggtttactcctgggtcggcaccagccgccgccggctgtccctttcacagaggataaaagtactcgctctggagttggactttaatgttgtcatgaaacctctggcccagcagcgggctccgcagtgggtggcaggtgaaggcccctccccgggcctctccaggcaggtgccgcctggccagcagggaaggcaggcagtgtcatcccccactggctctggggctcaggctacctcctgctgtggccggaacatctcccccagtggtggagcccagtgtccgtgaggccagctgggcctgaaaccttcctctctgaagccccgctgtccccttgccctgtatggagggcagaggctggagcgcaagttcctaggatgtgcttgcgagacccccgagcccaggggcgaggcccatctcagcccacccccgaactggaaacccttggagctctgcccctcgtggtgtgaggcccctgctatgcgaccctcagccctgccagcaacggaaggtgcagggcccgggcccacgggcttaacgcaactgggcctgggtcacctgcggggcctggtcccaggaggaagacccaggtgccaccctcctgggtgccacgtccaggtcacgtggggacccgtccatgtcacagaagatgcagggtcacccggtgagctggcgccgggccctgccagagcaccagccgcgggtggaggtgggccccagctctcctgtcaggcacgtggtgctgggaggtgcggccggagcagtgcccaccagctgcagcaggacaggtgggcacaggcccaccagcagtgcccgcacgggatgggcccctgcaagggccagagaagccacgctcctggctgggggctgggctgggactgacaggtggccctgccctctgcgccccactacttcccagccacccgggactccaaggacttgctgagctgggcaggtgggacgccgaggggagtcaaactgctcgtgggggcaggaggggcggtccacagggctgagccctgagctgaaccctggccctgctcgtggttgtgggggtgggggggtccagtggcgccctagccctgctgaggcccagctgggacgtgcgcgccggagggcgaggggccagcccatgccatgctgtcccccgttctcagctccatgctaccactttgaagaaacagaacctgttgcctttttatttagaaagtgttgcttgccctgcctggggcttctatacaaaaaacaaacacagctcaacgtggcctctcctgaccagagacgggcggtggggactggggctcagcagacggaatgtgtccccggcggcgggagaccaggaggcccctggcccgctcctcaggacggctgggctgtccccacctggtcccctccgagccagaagatggaggagaggtgggctgatctccagatgctccctgggagccaagcgccacggggtggtcaccaggccggggccgtgttggccagacgcctcatccgcctgtgggagggggagggcagcaacccccggatctctcaggcaaccgagtgaggaggcaggagcccccagcccctccctcggccgctctgctgcgtggggccctgaagtcgtcctctgtctcgcccccctccccagggagagtgagcctgttctgggctgtggtcagacctgcccgagggccagcctcgcccggggccctgtcctgcctggaaggggctggggcagcaccttgtgttccggtcctggtcccggatcttcttctccatctctgcatccgtcagggtctccagcagcgggcaccactggtcagcgtcgcctgtgttccggatggcaatctccaccgtgggcagggggttctcactgtggaggacgagagaggtagacggctcacagagcagctgcaggagaggcccctagaaagcagtgtccaccccgctgcgggcagacaggacatggagcctggtttctgcacccggctcccgacacagggcggccgggcacgctgccaacatggcatctccgggtctgcatgtggggaggggtccacaggacagtgctgcaggtccagccattcccagtggacttgctgggaggaggagggccgtccgccccgctcagtgtccaggagaaaggagagcaaaggagtccatccacccaggagtggagtcccagggcccctgccctgaccagcctgcagggggcccctcggcccacatcacaggggcccagaatccataagccctgactgctccaccccggggcccctcaaagacgcgcctagactccgtccgagggccacctgcacaccctctggcgaagtggactcagggctgggggtcagcctcggtgaggccgcaaaggctggggactcctggccgagctgctgcctctgccaggagccaggcccagcctgccggcgagcctcagccacgccctcacccaccctgcccgcggcgccacgctggcctccgggtcctctcctctggcctcctgctgggccactggtgctcagccccagcagtcggcctgccaggagccctgcagagtcagcccccagagggaggagggggcccgggggaacagcacaggaacaaacagacccctggccttagttttagctcctcatctggaaaatggggacagtgtccttgctgcgaggggtttcagaggaccactgccatgcaacacccagcacacacccactgcgtgggggctcgggcccgagccggtgcccccgagtcccaggctggtggctgggccgccccagccaccctgccgacagctgcttcccagccgggcggtgctgcggcagtccagaagccagcactgcagacccaaatgtcactcctcacgttgcgggctcccagctgccttccttgggggcagcagacacgaaagtcaccaagcccacgccgacgggagcaaacacgtcttcctcttaaacaagtgcgggtcccggaggccctgtgtttacctccctgtggctccgggaagattgcatcccagggggttgttctaaaccaagggctgctcgggccaggcctggaaggaggggcctggagccaggagcccacccttacgggcattcggcttcctgggtctcaaggccggctgggaccctgcattcccaccacccgccaggtgcaagcagggaggccgtgtcggaggaggcagagggcctggagggtcgtcttcgacgtgacctcacttttacaacctcacaggtgcggcaggccagctgggaggcatggctgtgccctcctggtagatgagaacaagactgcagggagtgatccccctgaacttccccaaccaggaggagacaaaactcggtgtcgccctcctgcttaagatcaactgactctggacaaggggcccagcccacccgatggggaaagggcagtccttccaacaagcggtgctgggacgggacccggcaggccatggtttctcagctatgacaccagcagcacaagcaccccgagaaaaacagctaagctgggcactgtcacacaagtgaactccaaacccaagaaaaccacaaaaagcctgcggatcttcagatatgtgggaagggacctgtatctggaatgtataacgaactcctgaaaagtgaaagtgttagtcactcagtctgttcagctctttgcaaccccatggacggtagcctgccaggctcctctgcccatgggattctctaggcaagaatactggagtgggttgccatgccttcctccaggggatcttcccaacccagggattgaacctgtgtctctcttgcactggcaggcgggttctttaccagtagcgccacctgagtagaaacactccaggtgccctgagtgtcagagcaggagggactcggcccaggcctgtgaggggaccctctccgagtcccctgctgcacagcagtgagaggtgcgttctgagtcagcctccagggatgagggacttggtgtcgacatcactcccaggacctcaggatctgctctgggaagcgaggctccccaggctggccccaggcccgctggcctcagctcgtgagccgtgcgtggacaggtgccatgagcaggcctcccacgggactcggggcgcggcctggaccccggggctgccagtggtcgcggggggccccgtgtggcggctgttccctctcttgctccgagtcctaggaacatggtgggcgctgcctcctggggtttctggagaagcagctgagatgcaaacagccccacgcgctccctcagctgttccctgtcacgggtggccccttggtgacggcctccatgcagggacggtgacagctcgagcagccgcgtaaaaccacacggggacggtggcagctcgagcagccgcgtaaagcctgacatccaatttggaagcctcccgcagtggaagaggggcccggggacggggctgcccggggcgagctccaccgggtcgggggtcacgaggagcccacccgcgtccccgccaccagcacctgggaccagataccctccccgctctgagggcggcctgaacgccgccccctcccacgggggcgcccaccgcctgctcgtggactgaacaagaggcggcagtggcctccagaccccctcgggggagggcagacctgtccgagactgagcacaagtccagggaatgagcaagggtctcagtaatgtccccaccgggacgggacgggaggaggcgacagaggccgctgaggtgcggggcagccctcagtagctggcatcaaggccccaggcagtcccggggcatccccgcagggggcgggggcgaccaccggcccgagcccaggcagtcccggggcatccctgcagcgggcgggggcgaccaccggcccgagccctacctgaaggcgtaggtcttctgatgccagctcagctgtccccggatgctgtaggcgatggtggtgacgaactccccgcccagccccagctcggagcacagcttcagagcgaacttctcgggcgagttctccttctccgacatgtcccactcgaactggtccaccaaggagatgttccccacgtggatgttcagctggcccgggagcacagacatgagccagagcggccccctctggggccaggccgcaccctcaccaccccttctccccggaacatccccgcctcgttcttggccgcgcccctgtgctgctacttggggtaaggaaaacaacccccatctctctgaaaagggttaactagcgaggaagatgcgctggtaactggaaaactccctacaaagaaagcttggatctgatggcttcactggtgaattccaccaaacatttcaagcactaacaccaatccttatcaaatcctgccaaaaaactgaaaaggaaggaacacatcataactccctgccttgataccaaagccagacaaagatactacgagaaaggaaaggtgcagaccggcacttactgtggacattgatgtgaaacctcagcagacacgagcaaaactacattcaccagcacgtcagaagaatcacacaccgttataaatgatgggatgatgacacaaccacattataaacggtggggcttactctggtgatgtaaggacggctcagtaagaaaaccggtcaatgccatgaaccacttgaacagagtgaaggacaaaaaccacacagtcatcttgataattggaggaaaatcattagacaaacttcaacgtgctttcacgataaaagcactcagtaaactaagatcagatggaaaccacatcaacaagattaattcagtcaaaaaattcactgcaagtatcacccacaatggcagaagactggtaacttttcctctaagatcaggaacgagccaaagatacccagtcttgccacttttgttcaatatagcgttggaatttctactcagtgcagtgcagtcgctcagtcgtgtccgactcttttcgaccccatggatcacagcacgccaggcctccctgtccatcaccaactcccggagttcacccaaactcatgtgcactgagtcagtgatgccatccagccatctcatcctctgtcgtccccttctcctcctgcctccaatcccttccagcagttaggcaagaaaaataaatcaaaggtatccacctggaatggaagaagtaaaactatctctggtccgagatgttacaatcttatatgcagagtttaagatgctaacaaaatactattagaactaatgaatgaattcagcaaggtaccaggatacaaagtcaacgtgcaaaaatcagccgcatttctacatgctaacactgcacaatctgaagaagaaaggatgaacaaattacaataacataaaaaagaataaaatccttagaaattaacttgatcaaagagatgtacaatgaacaatataaaacatactgaaagaaattgaagatataaataaatggaaaaacatcctatgtccatggattggaagacttaaaattattaagctgtcaaggctatggtttttccagtggtcatgtatggatgtgagagttggactataaagaaagctgagcaccgaagaagtgatgcttttgaactgtggtgttggagaagactcttgagaggtccttggactgcaaggagatccaaccagtccatcctaaaggagatcagtcctgggtgttcattggaaggactgatgttaaagctgaaactccaatactttggccacctgatgcgaagagctgactcatttgaaaagaccctgatgctgggtaagattgagggcgggaggggaaggggacaacagaggatgagatggttggatggcatcaccgactcaatggacatgggtttgggtggactctggaagttggtgatggacagggaggcctggcgtgctgcggttcatggggttgtgaggagtcggacacgactgagcgactgaactgaactgaacatgaatacccaaagcaatctacaaagccaaatgtaatccctatcaaaatcccaatagcatttctgcagaaacaggaaaaaaaatcttaaaattcatatggaatctaaggaaaagcaaaggatgtctggtcaaaacaatgacgaaaagaacaacaaagctggaagactcacacttcctgatttcagaacttactgcaaagatacaataatgaaaacactgtgggactaacgtaaaagcagacacgtgggccaacgggacagcccagaaataaactctcaaataagcagtcaaatgattttcaacagagatgccaagaccactcagtgaaggaaagtgtttgcaaccaacggttttgggaaaaaagaacccacatgcgaaagaatgaagtgggacccttacccagccccatctacagaaatcaactcaaaacagacagaacatatggctcaagccataaaacgctcagaaaaacagagcaaagctttatgatgttggatttggcggtgatttctcagatatgacgtcaaaggcataggtgataagcgaaaaaataaactggacttcaccaaaatacaacacttctatgcatccaaggacactaccgacagcataacaaggcagcccagggaaaggaggaaacatccgcaaatcacagcatctgggaacagaccgctgcctgtgagatacagggaaccgataaaaacaagaaaacagcaaaacccggactcaaaaatgggaaggactccagcagacacaggagacagacaagccgccagcaggtcactaatcagcaagcaaggcccgcaaaggcccgtatccaaggctgtggtttttccagtggtcatgtaggaaagagagctggatcgtaagaaagctgagcgctgaagaattgattgaactgtggtgttggagaagactcttgagagtcccttggactgcaagatcaaaccagtccattctgaaggagatcagtcccgaatagtcactgaaggactgatgctgtagctccaatactttggccacctgattcgaagaactgactcattggcaaagaccctgatgctgggaaagattgaaggcaggaggagaaggggacgacagaggatgagatggttggatggcatcactgactccatggacatgagcttgggcaagctccgggagagagtgaaggacagggaagcctggcgtgctgcagcccgtgggtcccaaatctttggaccaagcgactgaacaataacaaatcaacagggaaatgcaaatcaaaaccacagtgagatactgtccaccaccaggcaggcgttcttcagcggggttcggggcaggtggtgccctcttctctcgtaacgcccccaggaccgcgggggctgctgagacagcatggggtgtgcttggcctagcctgcccatgacaagagtggcagtgtgctcgcctcactgcgcccttccctgctctgcccaccagctgggccacccctgggaccacccagcttccgctccgtggacggcaaggccgcagcagcgcccggacacgcccagaacgtggtgccctcctcagaagtcggcctgtgcccttcctgggacaagccgcccaagagacagtcttccagagccctgccccacaacacggaccccagacaggctcctgtggaggcctccacgcacctccgcacctcgcaagccccgaggacaaggcaggcccgctgcgggtgaggagccgcctaccttgataatgacgcgctggtctgactggtcttccaggatgctgtccgtggggtaggactcgatctgctgtctgatggcagaggcaatggctggcacgaatgtcagtgggttcagatccaggtcgtcacagagaatctctgagaacatctccggggtcatcagcttctctgaaacgatgacggagcgggggaacccccagtggaccacagggcctacggtcagcgtgctcagccccggcctcccccagccttgcctcctctgccaccgcccccccgggtgacgacaggaccccctggcagcacgcagacagagctgagtgcacgccagccagggcggcggacggaccattcatgttccaggtaaaggcatcccgcagcttctgcccgtcaatctccatgtccagtcggatggggaccagcacctcgggctgggacgcgttctcgtggatcacggctgggtcgtggtcgtcgaagctggaaggggagcggccgcgtgctcagcaaagcgggctgggcccctgtgcccagggcctccctctctgcaccactggtcgctgagacctgcccagagaggacctgtccactacgggccgggccggcagaaacagggctggcgggggtccacgcggggcgggaggggagctgccgactcggcagcgggacaagctcagaggttccctgcaggaagagaggtttaagccccagagcaggcaggattctcccagcagctgtggggaagaaagggtatgtccagaagaagaaaccctggaacaaaggccgaggggcaggagggttgaggagctgcttggagagcagtgaaggggggctgggcggctggggggtgctggggagcctcggtggccaagcacccagggctccccacctgcagcctggaccccgagggagccccagaggacggagagcaaggcagctccgcactcacacctgccctttaggatggggaagagggaagagacgggggctgcggggggcaaggaaaccaggcacgccccgcttagacccgggggcgagaaccactttccaagaacgcaggggcgccaatgatgaacaatgggtagcagcccgcaggcgggaggcccggtggccgaggcccctcaccagagcgggaaggtccgcttcttgtcgcggcccatgcggttcctgttgatggtggtggagcagggcacggcgtccaggtggtgcgagctgttgggcagggtgggcacccactggctgttcctcttggccttctgttccctgggagacacagacgcccgtccgctcagcctatgggccaaaagccgccccccagccgccaggttgtggccagtggacgcccgccatgcccctctgggcccaggcccccatggggacctctgtgcgcccagctccgcggtggttattccccaggctccaagcggcacctgctcggggtcaccagttttaggggaggaggagagggcaggggccccagcccagtctgtgagctgtcacccccaggctccaagcggcacctgctcggggtcaccagttttaggggaggaggagagggcaggggccccagcccagtctgtgagctgtcacccccaggctccaagcggcacctgctcggggtcaccagttttaggggaggaggagagggcaggggccccagcccagtctgtgagctgtcacccccaggctccaagcggcacctgctcggggtcaccagttttaggggaggaggagagggcaggggccccagcccagtctgtgagctgtcacccgtgctatgtgctgggctgggcactcaggaaagagggtcagggttcacgggggggtggcgcgcagatttccaggagagccccgagggcagcagagaggaggctcaggtcaatggttgggcagggggccagggctggagacacagagagggtcccgattcgggggggtgccctcagcaggtggctgggagtccctgggggtttgcacactttcgatcaggctgttatttcagacgcttggtccagcctgagacaggtaatgcctctggcctccgggccttcagggatggaaagatactctagaaagcgggactcaaagtaactcaaggaactcgcgtcccacagtggggagcccttctctccaatttacatggggcgtttactacgaggaaaataccgaaggccgttttgagctgaggctcccgggccgggctgtccgtttgtgagactgctcgtcacccctgggccacatccctggtggccaagggggcaatcagtgcggtgactgcacgacacacctctgcagccctgccccacagctgtcaccatcggtgacgtccaccccctggagaacctgaccactgcccggtttcccgctaaaacagcgcccttccaggatggggggcagagggagaggccttggccttttcactcctcttctgcagcgggggcccctcgcaccccagtgcccgggcccaggagcgccccttggggtggggcagggagggatccacacaccaaggggagccaggacccccccaaatctgctgccctgccctgatacccgagacctggggaaacgggggactggggctgatgcgggcaggaccaagaactgaggcggtgagacggggtccccaccacaggccatctggctggcagtttctactccgggcctgcaggccaagagggaaaaggtgccccactcagatcaggcgcctcccgtccccagggagggcctacaaggtcagatcctttgtaacttccacgggcaaaactggcttgctgggcctgtgcgggccgcatgggcgtggaccaccacacctttccccactgagtctccagccggagctgtcacccaggtccccccaggccagccccaccccgccaccttgcagtagcctctcgtatccaggccgaggctgcccggtcgacccctcctgcctgatggcctcaagtggacaatgcgagtcacgttgcagcacgtgagtgggacgggcagcgccacgcggggtccgggcatccgagtcccaccactcagcctcccttccgctgcagagaggtctgtccaagagccctgggggccatccagcccctgtccgacctggccggtgtggaagagggggtgtgccacccctcctggggggctggctgggcgctgggcaggcccctcctaagagtggagcccactggtggttttcctgcagccccacctccacacagcagttctcactgcccagtaacaggaggctactggcctagctctctccctcgtgtgatggactcaaccaggagcgttcacggccccacacagggttctcggctgctgcatgaggatctcaaagccccatccacgtgcatgtaatctcctccggtaacttctctagggaagcccggctatcctgccatcctcaccgcaccaccagggcgagaaaagccatctccagcgctcacatccacaatgggccaggccgtgagcacaccaccttcttcgggaggttgtgggggcgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcgcgccccccccccccgcggcgccggcaccccgggcggcggcccccggcgctgggagcaggtgcggggccgcggccgctcgtgagcctccagcccggaggacgggccccgggggccggcccggtgcccaggccctgggagccccggaggccagagtgccagagggccggaggacccgggaaggcccgagagaggtgggaagcacggggttccagccctaggccatttcagccccaaagccatcggtgaaaccattgctggccccagataaaagcgtcgccaactttttcaccccggcggagactttagcgggtagctgccccctagggggaatggaaaaaccaggatttaccaggtgggtggaggtcacaactgcccagatcctgagaaagaggggtcagtggggcgggaagattagtggggagaggagctttcagaacccaagggaatgaaacgaggcttgaggttggttatccagcagccgccccctgccccgtgagtgagcgaaggctgggccccttattgtcacatcttccagctcttcgctagaaaacctagagttttaaatactgtggcagctgagtcaaacaataaggaaaagcccgactctttgagagccaggcacaaggcgtctgtgacagggtctccaggctgcccatttgcagtctctgaaacggagggtttttcgagaaggaggtcttggggtgcctgccagaattggaggggggggcgcgggaagtgaggacccagaagagagggcttggcccgctgcaaggaggtcactggacactggagctgaagcgccagccgaaactggaaactcgaaatctgtctccgtgccagccacaaggcctatgattttccttggcgacgttcagcatcttaggaggagctggcgggggaggcgggtagttcgtgggcggttgcagcagggcaggaaggtgaggaacctgaggctggtcagagagctggttggagtgatgcccatcggtggacccgctggagaaggcctgagtagagaaggtctaagcttaacggggaaggggtgggccagggtggaaatggggtgggaagtttgaggagggggagcagtggagatgggggttgtgaggaatgggagtgagcttagacgtcttgaggatactgcagttctgtgctttttttcacacctggctgaaaattcactgaaaacaaaacaacccttgctctgtgacagcctagaggggtgggagggaggcttaagagggaggggacgtgcgtgtgcctatgggcgattcatgtgggtgtacggcagaaagcaacacagtatgtaattaccctccaattaaagatcaagtacaacttaaaaaccccaaacacaacattgtaagtcagctagactccagtaaacatttcagtaagaagattcaactgggaatgagttccgccgtgactatcctgatgaatttcccgtgtcttcttgaggccattcctctttgaacttccgtgtttggggaagcgtgcctttgtatggagtcctgaggagtaaatgagacgggcttgtagaaggcctagtagtgccttgcacgcggcagatgctcaataacctcgagttgtcaccattatggtacctcaagagtctccttggagcttgcacggtttctgaatggggtcctgcggggctcccttggggctcccacatggggttggggggctgagtggggtgtccccgctccttgcttgtcccctgtggaacacccccttccacccgagcagctctgcttttgtctcttgtgtttgtttatatctcctagattgttgttcagtcgctcagtcgtgtccaactctccgaccccatggactgcagcacaccaggccttctgccttcaccatctcccggagcttgctcaaactcctgtccattgagttgctgatgccgtccaaccatctcgtcctctgtcgtccccttctccttttgacctcagtctttcccagcatcagggtcttttccaatgagtcagctctttgactcaggtggccaagtattggagcttcagcttcattatcagtccttccaatgaatattcagggttgatttcttttaggattgagtgacttgatctccttgcagtccaagggactctcaagagtcttcaacaccacagttcaaaagcatcagttcttcggcactcagccttctttatgatccaacgcccacatcggtacatgactactggaaaaactttggctcagagataattgacttgattgaatacaaagttctttggcaaaaaataaaagtgtggcaagcagtactgacacaaaagcaagtggcttttcctccgttgagtcatttatttattcagtgggtgtgtgcgtgtagagacggagcggctgtgctgggagctggggcttccacttcagaggagccccggacctgccctcggggagttcacaggcagtgctgcggggggtcctgccaggacgcctgccctgcgagtgcccagtgctgtgatggatgcgtgtcccgcatctgcggccactggggccacgtgcccgagattgtccgggtctgagggtgcagagaagaggaggcatttggactgagtctggaaaaatgagcatgtggccacgtgagaagccagtggtgaggggaccagtcaggcggaggaaagagcggctcatacgagttgtggagctggaagcatgagggtgtgtggaagcagaggccggggacagggccgcagggccggccatggagggcgtgggctgctgcaggctcctgagaagggggacgctgccatcatgaccgggtttaggtgtttgaccctggtgtccacgtagaggacagatgtgtggggggggagctggagatgggcatccatcgggagtcagcctggagagaggcagagaccccgtcagtgggccctcaggacgtggatggggcggatgttgggaagatctgactcctgggttccggctggggctccgggctggaggggtgccgcccaccgagcacaggaggcaaacagatgccctctcccagcaagaccccagccccagcaccctccggggccggactccgcccctcttccagaatggctcccttgctgtcctcgcccatctttccggtgccctgagcctctagagtctggacaccagcgtccgccttgcgcttgtttctgggaagtctctggcttgtctctgactcacccaggaccgtcttcgagggcaaggttgtgtccttggttccatctgctttggggtccggctcctcgctgcttgacctgctgatgtgacagtgtctcttgttttcttttcagaatccgagagcagctgtgtgtgtcccagacagacccagccgctgggatgacgggcccctctgtggagatccccccggccgccaagctgggtgaggctttcgtgtttgccggcgggctggacatgcaggcagacctgttcgcggaggaggacctgggggccccctttcttcaggggagggctctggagcagatggccgtcatctacaaggagatccctctcggggagcaaggcagggagcaggacgattaccggggggacttcgatctgtgctccagccctgttccgcctcagagcgtccccccgggagacagggcccaggacgatgagctgttcggcccgaccttcctccagaaaccagacccgactgcgtaccggatcacgggcagcggggaagccgccgatccgcctgccagggaggcggtgggcaggggtgacttggggctgcaggggccgcccaggaccgcgcagcccgccaagccctacgcgtgtcgggagtgcggcaaggccttcagccagagctcgcacctgctccggcacctggtgattcacaccggggagaagccgtatgagtgcggcgagtgcggcaaggccttcagccagagctcgcacctgctccggcaccaggccatccacaccggggagaagccgtacgagtgcggcgagtgcggcaaggccttccggcagagctcggccctggcgcagcacgcgaagacgcacagcgggaggcggccgtacgtctgccgcgagtgcggcaaggacttcagccgcagctccagcctgcgcaagcacgagcgcatccacaccggggagaagccctacgcgtgccaggagtgcggcaaggccttcaaccagagctcgggcctgagccagcaccgcaagatccactcgctgcagaggccgcacgcctgcgagctgtgcgggaaggccttctgccaccgctcgcacctgctgcggcaccagcgcgtccacacgggcaagaagccgtacgcctgcgcggactgcggcaaggccttcagccagagctccaacctcatcgagcaccgcaagacgcacacgggcgagaggccctaccggtgccacaagtgcggcaaggccttcagccagagctcggcgctcatcgagcaccagcgcacccacacgggcgagaggccttacgagtgcggccagtgcggcaaggccttccgccacagctcggcgctcatccagcaccagcgcacgcacacgggccgcaagccctacgtgtgcaacgagtgcggcaaggccttccgccaccgctcggcgctcatcgagcactacaagacgcacacgcgcgagcggccctacgagtgcaaccgctgcggcaaggccttccggggcagctcgcacctcctccgccaccagaaggtccacgcggcggacaagctctagggtccgcccggggcgagggcacgccggccctggcgcccccggcccagcgggtggacctggggggccagccggacggcggaatcccggccggctcttctctgccgtgaccccggggggttggttttgccctccattcgctttttctaaagtgcagacgaatacacgtcagagggacgaagtggggttaagcccccgggagacgtccggcgagctctaacgtcagacacttgaagaagtgaagcggactcgcagcccgtacagcccggggaagatgagtccaaagtcgagggtcaccttggccactgcagggtcgctcggcggtggggcggagcgggtgcaggagggctcctcctgggcttggggtggcaggcgaggaccccgcgcctctcagccctcggcctgggttggctgagggcgggcctggctgtaggccctccagcggaggtggaggcgctgcccggctcagccaggcacaggaccctgccacgaggagtagccctccgccagacccggcgtccaggctggggcgcctgcggggcctccgttctgtggctgggcagcctgcgccctgtccagggatgaaggggttccggtctgaagggctgggttcagggtccagctctggcccctcctgccttggtgtcctggaggaagccccaaggctccgtttccctctccaggaggtggggacgttgggaatgccacattcccctggggggtgtgtgtgtgtgttcaaggctcccattcagactgggactgggcactcacgagctttggcaactggcaactgaggacggagacccagggtgacaccccacctcctgctgcggcccccccggcaggggagacacaggcccgtctggttcccaagatggcagggcccctccccctccagcttgtgccctgggtgtggtgcctggggctacagcgaccctttccggttccccgggccagttcagctgggcatcctcagggcggggctctgagggtgccatgtttccagagctcctcctcctcccaccagtagcaggcgggcggccagctcccaggcagccccctggcatcgcctaggtgcacacctgcccgctgtgacccagcaaggcttgaaggtggccatcccagttaagtcccctgcccctggcccaggaatgggctcgggcagggccgcatctggctgccccagaagcgtctgtccctggcctctgggagttggcggtggtctctggtactgtccctcgcagggccccttagcactgctcggggaggaggtgggctgaactgattttgaagttttacatgtctgcggccgcagtcctacgagcccgtcagggtcatgctggttatttcagcagatggggcttggctcggcagctaggatggtcctgaataaaaatgggaaggccagagctgttcctccatcagcaggcttggcagctggggacgttgaaaggacaggtctgctggtctggggagaccagctctgtgcagcccctgctgtccgtgggggtactaaaccagcccctgtgtgcgcccatctgagtggcagcccgcctggaggatcgcccatcacttgtgagaattgagagaatgctgacacccccgcttggtgcagggggacagggccccctaagatctacctccttgccccacccccgggaccccctcagccttggccaggactgtccttactgggcagggcagtcatccacttccaacctttgccgtctcctccgcgcgctgtgctcccagccaaattgttttatttttttccaagcatcactttgcacacgtcaccactctccttaaaaccacccttccggagtctcctgctcgtaaatcgccggtttcagccaacctgggtcgccccccaagcccagcaagcctgctgagccccgcgcctcccagctacttcacgctcgcctcaagcttctaaacgcggaccttctcccccccacccccatccctttcttttctgatttatgtaacacggcaggtaagactcctctcctgaagggttgacagactcacacaaaaccgtggtcagaccaggcaagtgctttttttcagaagtgtgagcggaacctagtcttcagctcatgctctttccttgttttcttatgtgttctaagtcctttgacttgggctcccagacagcgacgttgtaagaggccgtcctggtagcatttgaattgtcctcgagtttcgttgtcggattttgttttattgtcttagttttcccttcttttagcagacgttgttgactgtcgtaaagctccagttcttggttctgtttactaatcaaattgttttgtcaaagtacatgtattctgctcttttctttatcttttttgttgcttaatattaacactttacatttctaagattaattatttaggtaattaataatttttaacatttctagtaaacgtgggtacttgggtctgtgtttgttttcttgtagttacagctttttctgctctatactgttgacgtctgggtttttttttgctcttaggaatttccctttgaccccattattattattttaattagtattttttaataattaaaaattagtgtttttaaattaaccctaatcctaaccccagtgatgactgcttcagtcattgctgttacttattatgtgctggtgtcaggatttttaagtgtccatagacattctctgagcctgaatatattatcagttttatacagcatttgtgtactctcaagaaacgtgttttcactctgtcagttcggtttgttacctcagtctttatgttattttgctccagtccgcacttgctctaacttgtcttcccttcgaggtgtgaggacgcctggcagccggtgagcatgccggggtccggggtcgtgggcccaggcgcccagcaaagccctgtgggtgtgtgcacggctgggctgctccgggaggaagcctgtggccccacggtagttaggagcgctggtttacctggtcacaccacggtctggttttgtgtgcttttccctgacgtgtttctgttttgccttggtttctattctgttttatgagtgccgtttacgctttgttagtcatgccgttatctcgatagacagggtgtacgtgatcaagtgattaccgtatttggagcagatgtctatttaacagagatgaactgagaacctgtgcctttgcatgccctctttgcctcttttaatgcttctagcttcaacttctcttttccaaacattataatggaaaccccttgcttttttttttttaatttgcatttgcatgagagtttatttagctcggcattttatttttaaaatttgtgtatatatttttgctatatatctgtaacttataaacagcaaattattggattttgctttctgattctttctgtaattcttcttacataagaagttctcctatgagtaacattgctgtttagagtgaggcatgatttatttccagcttagtatgtattgggtcggttaacccccaaaggtcatgctcatccccgccccatctctgtgagttattgtccgagtgtggagcgccctgtctaggccgacgagagacccaccatcgggcacacctgcccctcctggtctggtcagtgccgggctctgtcctgagtccactcctgatgtcacaggctggtgcttcagcgacctcggctgtgacacggagggtgtgatggcactgcccagccccatggggcttggaggactaaaggatgcacacctgcctggcagactgagggcacaggtgtttctcacactgtcagcgttttgaaatattcctttgattttctaccctaactcccaaaggccgttcaacataagctagaatgctacgtggtgcttgattacattttagaaaagtttcagcaaataccacgagatgcagcaaagaactagacctcacagatcaggccgcctgcataagggagcccacacagtcgtgggagacggggaccctctcccacgtcctgtctgtcccaggatggtcccctcacccgccccctctctcccctcgccctcctgtggtgggggccggccaccatcacagctgcagagcctcaagaagggggtcgccctggccactcccgtggcaggagggacacgagggcaggagcttaccgcgggtgcagtggtctcggatcagctcagctggccgctgcggggtcggggggacagttcagtgggaggcaggagcccccactacagctgccaggacttctcagaggtgacaagggggttcagtcacctcagcccaggtggaaaccaaatggcctcttgcgcggctcctggggccacgcggaggttcgctgggatcacaggtatctggatgtgtgcgccatggacatgcaccaccttcggggggtaaggggtggggaaaggcagcccctttcttttgggggaccccctcttcagtgtctgataaccaggaaaccaaatcagaaggtggtctgggggtgctgagcagggtgtctcctacaccacaggccacacactcacacagcctccaggactccagtggggctgagcgctggagactcacccacgtttgctacccccccacccaaggccatcccagaacagctgcctgcgtcctcacggctggcccctcccctctggtctaacccagtgtgggtgggccggcctggggtctccacctgcctcctgctgttccctgggctgctggctgtctgcagatgcggggccctggcccggagaagccccatcagagcccagaggacgggagtggagcggggaggtgagccccggagtctcgaggggccagaggcaaaatactgggctgtgtccctggaaggcagtttcccatgaaaccttcaatataggccgccccagacgatcagcctcatctgctacgtggattcctccccgtagcgaatggtgattgggttctacatggacccgggacttctgtttgaattataatctttcccccactgcccctccagggatctggaaaatggaggcctgggctagacggaagcttcctccaagattctttattgaagggattcgaagagaaacaggtggtcagtaatctgtgggggatggaggggtgagcgctacgtgtaacggttttactgttgctacgggaccagttttgatgtctttccccttcaagaagcagacccaaacaccgagatgctgaggttagcagcacagagcgggttcatccacaaggcaaccaggcagggagaccagagacgctctggaatctgcctccctatgggcacgggctgggtgctcacggatgaagaccaagcagcaggtggcgtggggcgtggggagcctgcggaaagcgatggacaaggtgcgggaccgcggtccgcgcggtggacccaagctccgcctctgcgctgcagcgcgagctgggggcggagcttccagggacccgcgaccgcgcccagtgggagggtccgcggtccacccagtcctaacagctcagctccagctagacgccgctgagtccggctttctagagagcaaccccggcgggtattttatggttctggcttcctgattggaggacacgcgagtcttagaacacccttgattagtgcgggcaggcggaatggatttgactgatcacgatctgcagtttcaccatctcaggggccgccctcacccccacctatcctgccaaagggggggcctcggtgctgagatcggggccacacgtgcactagacggtcggtcagcgctgctgctgagcggacccggggccatcctcacaccgccactggcccctgtgctcaataaaaggaaggaaagcgggaaaagcgctttctggccgcggtggcctcgcgcgttcctccatcgccatctgctggcagagcccggcatggcacccgctgcacagaaacctcggtgtccgtttgggtgccccatccttgaccccgagagagcaccctccgtccaaaatgaaaaacagctgctcccaagagtcattataatcacagccaattgtgttaattcgtcctcggatccactcacagttccacggaacattctgctaacctctgacaactcctacataaagcaatactgagaagaaaagaacgtggttgataaatacaaaggcatacaacaataaggagcaaagaaaaaagacagtcctcgcagttctgttttgttcatctctcatgagtaggatggcagataaaacacagaatgcccagtgaataattttagtctaagtatgtccccaatactgcctaatcttcaaatctaaccttatttttaaaatatatattttttgctggtcactcatcagttcatgcaccaaagcctttgtttcttgactcctaactttttgacccctctggggtgaggagcacccctaacctcgagagcccatcacacagtccccttgggactagacccttctttgcccatcacagctgaccggaagggccagcccatggccagcgctcgcgccccctggcggacagactctgcgcggcagccccgggagcccaggtgcgaccccgcggtctctggcgccctctagtgtggaaagatctcctcctggtgttcccagtcattgggctgtattttattagagaagatgctcgcgtgacgatgatgatggtcctttaccgggaggcacgtttggggcgcgtcggctcaggggccgagctattagcctgcatcgcgcccacaggcatcgcgtccccctgagccgggtcagctgtgggctgtcctgacacgggtttcccccagtctctggcccgctgtccctcccaggtcagtgtccagcgttgcccttctggttgtggacttgtgcagcggtctcagcagatggaggggcgaccctaaaggatgtattgaggcatctcagcactgtcctccgcccaggtttgctggtcagcagtgaagtgaccgggaaaaggggctgtcttggggtcctttcagaggcctgggttagaccaaagttttctagaagattcaccattgcagggagtcaaagacaaaactagggtggtcagcaatctgtgggggattcggcggtgagggaattctgaatgctacatgtaatggttttactattgttagggaacatttttcccccctacaaacagcaggccaaaatactgagatgtcaggtttgcatcaaagagcgggttcatccacaaggcaaccagagaacgctctggaatctgcctccctgcgggcacaggctgggtgctcacggatgaagaccaagcagcaggtggcgtggggagtggggagcctggggaaagcgatggacaaggtgcgaggacctccggcgcgagctggaggcggagcttccagggacacgcggccacgcccagtgggagggtcagcggtccatccagtcctaacagctcagctccaactagacgctgctgagtctggctttctagagaacactccgggcgggtattttattgttttggcttcgtgactggaggacgttcaagtcttaaaacacccttgattagtgcggggaggcggaatggatttgactgatcacgacccgcagtttcaccatctcaggggccgccctcaccccctcctaccctaccaaaggtgggggcatcggtgctgagatctggggtgacacataaaatcaggtgaagtcttaggacagggggccgattccaggtcctagggtgcagaaaaaacctacctggccccgggctagacagcgtggagggcgtggcccgggctggtgcacagaagtggcccccaactggtcagaaggtgtgggagcccagggctggtctactgcagaaggggtcgcctggtggacagagtggggcctgagtgcctgctgaactggtccgtcagggctgctgagcagacacgggccatcatcactggctcctgtgctcgatagaagggagggaaaccaggaaagcaaaggcgctttatggccgcttttgtgtttcgcgttcctctagcaccgtctgccggcagaacgcggcattacatccgctggccaaacctcggggtccggcttggatgtccccatccttgtctcggagatctcacctctcagcagttcccctggggacaatgtcgagaagatgcgaccttgacccggagctcggtggagagggtgccctgggttctttccgcagttgcttggagtggaggtgcctcatgttgggctgggaacgggaggaaggaaacaggtcatgattgagatgctctagacagactgtccctgctcttgccaaatttcagaagattgtctttaataaatattccattttttgtatgcccttaggtctatttccagacactttaaatatattgaaagactttaaatatttatataaaaatattatttatagactgtataaaaggaacagttagaactggacttggaacaacagactggttccaaataggaaaaggagtacgtcaaggctgtatattgtcaccctgcttatttaacttatatgcagagtacatcatgagaaacgctgggctggaagaaacacaagctggaatcaagattgccgggagaaatatcaataacctcagatatgcagatgacaccacccttatggcagaaagtgaagaggaactcaaaagcctcttgatgaaggtgaaagaggagagcgaaaaagttggcttaaagctcaacatttagaaaacgaagatcatggcatctggtcccatcacttcatggaaatagatggggaaacagttgagacagtgtcagactttatttttgggggctccaatgaaattaaaagacgcttacttcttggaaggaaagttatgaccaacctagacagcatattaaaaagcagagacactactttgccagcaaaggtccgtctagtcaaggctatggtttttccagtggtcatgtatggatgtgagagttggactgtgaagaaggctgagcaccgaagaagtgatgcttttgaactgtggtgttggagaagactcttgagaggcccttggactgcaaggagatccaaccagtccatcgtaaaggagatcaccccctgggtggtcattggaaggactgatgttgaagctgaaactccagtactttggctacctaatgcgaagagctgactcattggaaaagaccctgatgctgggaaagattgaaggtgggaggagaaggggacaacagaggatgagatggttggattgcatcactgactcgatggacgtgagtctgagtgaagtctgggagttggtgatggccagggaggccctggcgtgctggcggttcatggggtcgcaaagagtcggccatgactgagtgactgaactgaactgatccagaaatttaaaattaatatataaaccaaatccatgcagacaattataagcatatattataaatgcataattataagcaagtatatgttatatttataatagtttataatgtatttataagcaagtatatattattataagcataattgtaagtagaagtaactttgggctttcctggtggctcagacagtaaagaatctgcctgcagtacaggagaccgggttcgatccctggtttggggaaattccctggagaagggaatggcaaccaactccaacatgtttgcctggagaattccatggacagaggagcccggaaggttgcagtccatggggttgcaaagagctggatacaacagagtgactaacacatgtatataaataaatttacctatatattgtatatatatttataaacatattcagatattataaataattagaaacatattatacatgtatttaaatactgttataaacataaatttaaaaaataattttcagccctttggcttgggggtgtgtttgtggacgtctttgtgctactgttcctgaagtggagctctcccctcccaaaccagcttttgaaatgactgggaaagcaatggaatacataagcatcaggaagatagcaacagagctgtcattcttcacagagggtgtgcttgagtgtgtagcaagtcccgcagaatgtagacagattaatatagtctattaaaaatagtgtagcaaatttacgaggtgcgatttcaagtataaagacttactgggtctctcagttcagttcagtcgcttggttgtgtccgactctttttgaccccatggaccgcagcacgccaggcctccctgtccatcaccaactcctggagttcactcaaactcatgtccatcgagtcggtgatgccatccaaccatctcatcctctggcgtccccttctcctcccaccttcaatctttcccagcatcagggtctttcccagtgagtcagttctttgcatcaggtggccagagtagtggagtttcagcttcagcatcggtccttccaatgaatattctggactgatttcctttaggattgactggttggatctccttgcagttcaagggactctcaagagtcttctccaacagcacagtctatgaatagaatagcaaatgaatagagaataacatttacgaggatatattttaccattgcataaaatatatcagcttgtagagaacagacttgttcccaggggagagggtgggtagggatggagtgggagtttgngatcancagaagcgagctgttatatagaagatggataaaaaggatacacaacaatgtcctactgtgtggcaccgggacctatattcagtagcttgtgagaaaccataatcgacaagactgaggaaaagtatatatatatgtatgtacttgagttgctttgctgtacagaagaaattaacacaacattgtaaatcgatatttcaatagaatccacccccccaaatatataagtttcctggagatggagacggcaacccactccatttcttgcacccaatattcttgcctggaggatcccatggatagaggatcgcaaagactcggacataacccagcgactaacactttccctttcaaatgtgtaggtttactagcgtgaatctacagagatgcccaagacattcgtttatgaggaaaactccacacgcagcttcactgagaattattaaacctattaaagggagagagcgccaggatattcatggattgaaagattcgatgtggtcaagttgccagttttccccaaactgattggtaaattccccaggagctggctcaaggcgcaaaattccctttacctttttttaagagacgaagccaaggagccgattctggttgagagacgctcaggtcctcctgcgggagagcagccctcttcctcccggtcgcctgggcagtttcgaggccacgaccagaaggacttggctccctgtgtcgcgcactcagaagtctccctctccgtcccaaggactcagaagctgggcgtcctgcccgcagcagaggaggcagcctggaggggccccgcgggcacagcggtccgggtttcagccgagttgcccgccccgcccctctacctgggcgctgccgcccggctccggggccggccgtgccctccgtggccgcaaggcgtcgctgtccccccgctggaagtgctgacccggaggaaggggcccagacggagggactcggagcctccgagtgacaccctgggactccgagcgctggagcctggcgtcaccccaggcaggggcagtgggggcccggggcggggtcaggggcctcccccggttctcatttgacaccgcgggggtgcgctgggcacagtgtccaggggccacgttccgagcaggggcgcgatgcaggcccgggcgcggcctgtcccgggcgcgagtccagctgctttgcagaggtggcggcaggtcgcagtgaccctcacagagacgccccactctgcggctccaggtgggcctgtgccccccagaagtgctgacctgtgcaccgggaaggcacagggccccccagccatgtctgcgatggaagagccggaaccgcgccatgcccgtcctcgctgaccggcaggcacccgccgtgtgtccacacgctgagccatctggctccccttgcttgacatacacccaggacctgagtgtgcaggaagttagaaggggcaggtgtggtgacacgatgccatccagcatcacctgagaacctggacaaacctcaggggcccagcctgctctgtgaggccccgagggccggcccctccccggacccctgccttgaatccggccacactgcccgccttcctgctcctgcggcttgtcagacacgcctgagcccagggcctgtgcactcgctgtcccttctgccaggactgctcctccccaggctcttgctggggctccccttcttcattcgggggtggcctctcttgttcagtggctcagctgtgcccagtctttgcaaccccatggactgcagcacgccaggcttccctgtccttcactagctcctggagtttgctcaaactcatgtccattgagtcagtgatgctatccaaccatctcatcctttgctgcccacttcttctcctgctctcaatctttcccagcatcagggtcttttccaatgagttagctctctgcatcaggaggccaaagtattggagcttcagcatcagtccttccagtgaatatgcgaggttgatttcccttagaattgactggttggatctccttcctgtccagagaactctcaagagtcttctccagcaccacagtcggagagcatcagttcttcagtgatcaggtttctttatagcccagctctcacatcggtacatgactattggaaaacccatagctttgattagatggaccttcattggcaaagtgatgggccttcattggccctgctttttaatacaccatctaggtttgtcgtagctttccttccaaagagcaaacatcttttaatttcctggctgcagtaaccatccatagtgattttggagcccaagaaaataaaatctgccactgtttccactttttccccttctatttgctatgaagtgaggggactggatgccatgatcttagtttaaaccagcagttgtcaccccgaccgcttcctttcctaaagagctcatcacacctcccactggaatgcaatgtgttgcctgtccgcctgcttcacctcctgggactttgctgcaggtcttggtctctgaggcccctgccgtatccccagggcccagagcagtgctgggcttcgagtccgatcagggactatgtgtgtggactggatggtgcttgcttcttctggggaacgagagacctgggcctggggaacgaggggacctggtgtgaccggatctcctccctcgggagaggagccaagcgagtggacacaggtcagtgtgtcttgctcctgtgtggcaggtgtcccgtctgtgtctgtcatcttggcatttcggtgtttctgtgaacccagcccctcccctcctgataccccatcccatcagcacagaggagactgggcttggggactctctggtcctgagattcctctccgcatgtgactcccccctcctggggggagcaggcaccgtgtgtgaggagggtggaagcttttcaagacccccagcttttctgtcccagggggctctggcagggccttgggagctggaatgagctggaatctgggccagtgggggtttccctggtggtaaagaacccgcctgcccatgcacgaggcataagagacgcgggttcgatcactgggtcgggaagatcccctacaggagggcatggcaacccactccagtattctttcctgaagaatcccttggacagaggagcctggtgggctacagtctctggggtggcaaggagtcggacacgactgaagcgacttaccatgcacgcacgcggggtcaggggtcagggccgcgctgcttacctgctgtgtgaccttagccaggtcacaccccccaggctgtgaaagagaacagtcttcccagactcgggcatccaggtctttacagacgtgcctgtgagctttgtgactctggctctgtggccgctagagggcgctgtccgccgggccctatgtgcgtgcacgcatgtgagcatgttcgcatacgtgtgtgcatctgtcgggggcgcacggtgcggggacacgggcacgcggtcaggaacgcagcccggacacctccacgtggcccgcgagtaccgtcaggtgggggctgtggctccgctgtgtgggtgacccgccctccccccgcgaacgtggtgcatagtgaccgcctggctgggctcctgagctcagccatcctgccccccgggtcagctcccgacaggcccagctctaggccccaggcgtggaccgaggcccccaggccccggcctgtgagatgggacctccgtctggggggctcattctgctcccggaggcctggcaggcccctcctctttggcattgcataccctcgcattggggtgggtaagcacagtaccccatgcctgtggccccgtgggagcggcctgctcagggaggccggagcctcagctacagggctgtcacaccgggctgcagaggaagaagacgggagcgaggcctacaggaacctagccaggccctggcccactgagccgacaggagcctggccagaggcctgcacaggacggggtggcggggggggtggggtggggtgctgggccccgtggccttgactgcagaccccgagggctcctcagcttagaacggccaagcctgagtcttgggggtgcaggtcaggggg

SEQ ID NO: 32 - DNA, 10012 bp, Sus scrofa
gccacgcccactccatcatgcggggaggggatgggcagaccctccagaaagaagctccctggggtgcaggttaacagctttcccagacacagccagtactagagtgaggtgaataagacatcctccttgcttgtgaaatttaggaagtgcccccaaacatcagtcattaagataaataatattgaatgcacttttttttttttattttttttttttgctttttagggcctaatctgcagcatatggaagttcccaggctacaagtcgaaccagagctgcagctgccagcctacatcacagccacagcaacaccagatccgagccacatctgtgactaacactgcagttcacagcaacgccagatccttaacccattgagtgaggccagggatcaaacccacatcctcatggatactagtctggttcgtaaaccactgagccacaaggggaactcctgaatgcaatatttttgaaaattgaaattaaatctgtcactctttcacttaagagtccccttagattggggaaaatttaaatatctgtcatcttagtgcatctttgctcatatgatgtgaataaaatcccaaaatccatatgaatgaagcatcaaaatgtacatgaagtcagcctgaccctgcactgccctcacttgcctcatgtaccccccacctcaaaggaagatgcagaaaggagtccagcccctacaccgccacctgcccccaccactggagcccctcaggtctcccacctccttttctgagcttcagtcttcctgtggcattgcctacctctacagctgccccctactaggccctccccctggggctgagctccaggcactggactgggaaagttagaggttaaagcatggaaaattcccaaagccaccagttccaggctgccccccaccccaccgccacgtccaaaaaggggcatcttcccagatctctggctggtattggtaggacccaggacatagtctttataccaattctgctgtgtgtcttaggaaagaaactctccctctctgtgcttcagtttcctcatcaataaaaggagcaggccaggttggagggtctgtgacgtctgctgaagcagcaggattctctctccttttgctggaggagaactgatccttcacccccaggatcaacagagaagccaaggtcttcagccttcctggggacccctcagagggaactcagggccacagagccagaccctgatgccagaacctttgtcatatgcccagacggagacttcatccccctcctcctcagaccctccaggccccaacagtgagatgctgaagatattaagagaagggcaagtcagcttaagtttgggggtagaggggaacagggagtgaggagatctggcctgagagataggagccctggtggccacaggaggactctttgggtcctgtcggatggacacagggcggcccgggggcatgttggagcccggctggttcttaccagaggcagggggcaccctctgacacgggagcagggcatgttccatacatgacacacccctctgctccagggcaggtgggtggcggcacagaggagccagggactctgagcaaggggtccaccagtggggcagttggatccagacttctctgggccagcgagagtctagccctcagccgttctctgtccaggaggggggtggggcaggcctgggcggccagagctcatccctcaagggttcccagggtcctgccagacccagatttccgaccgcagccaccacaagaggatgtggtctgctgtggcagctgccaagaccttgcagcaggtgcagggtgggggggtgggggcacctgggggcagctggggtcactgagttcagggaaaaccccttttttcccctaaacctggggccatccctaggggaaaccacaacttctgagccctgggcagtggctgctgggagggaagagcttcatcctggaccctgggggggaacccagctccaaaggtgcaaggggcccaggtccaaggctagagtgggccaagcaccgcaatggccagggagtgggggaggtggagctggactggatcagggcctccttgggactccctacaccctgtgtgacatgttagggtacccacaccccatcaccagtcagggcctggcccatctccagggccagggatgtgcatgtaagtgtgtgtgagtgtgtgtgtgtggtgtagtacaccccttggcatccggttccgaggccttgggttcctccaaagttgctctctgaattaggtcaaactgtgaggtcctgatcgccatcatcaacttcgttctccccacctcccatcattatcaagagctggggagggtctgggatttcttcccacccacaagccaaaagataagcctgctggtgatggcagaagacacaggatcctgggtcagagacaaaggccagtgtgtcacagcgagagaggcagccggactatcagctgtcacagagaggccttagtccgctgaactcaggccccagtgactcctgttccactgggcactggcccccctccacagcgcccccaggccccagggagaggcgtcacagcttagagatggccctgctgaacagggaacaagaacaggtgtgccccatccagcgccccaggggtgggacaggtgggctggatttggtgtgaagcccttgagccctggaacccaaccacagcagggcagttggtagatgccatttggggagaggccccaggagtaagggccatgggcccttgagggggccaggagctgaggacagggacagagacggcccaggcagaggacagggccatgaggggtgcactgagatggccactgccagcaggggcagctgccaacccgtccagggaacttattcagcagtcagctggaggtgccattgaccctgagggcagatgaagcccaggccaggctaggtgggctgtgaagaccccaggggacagagctctgtccctgggcagcactggcctctcattctgcagggcttgacgggatcccaaggcctgctgcccctgatggtagtggcagtaccgcccagagcaggaccccagcatggaaaccccaacgggacgcagcctgcggagcccacaaaaccagtaaggagccgaagcagtcatggcacggggagtgtggacttccctttgatggggcccaggcatgaaggacagaatgggacagcggccatgagcagaaaatcagccggaggggatgggcctaggcagacgctggctttatttgaagtgttggcattttgtctggtgtgtattgttggtattgattttattttagtatgtcagtgacatactgacatattatgtaacgacatattattatgtgttttaagaagcactccaagggaacaggctgtctgtaatgtgtccagagaagagagcaagagcttggctcagtctcccccaaggaggtcagttcctcaacaggggtcctaaatgtttcctggagccaggcctgaatcaagggggtcatatctacacgtggggcagacccatggaccattttcggagcaataagatggcagggaggataccaagctggtcttacagatccagggctttgacctgtgacgcgggcgctcctccaggcaaagggagaagccagcaggaagctttcagaactggggagaacagggtgcagacctccagggtcttgtacaacgcaccctttatcctggggtccaggaggggtcactgagggatttaagtgggggaccatcagaaccaggtttgtgttttggaaaaatggctccaaagcagagaccagtgtgaggccagattagatgatgaagaagaggcagtggaaagtcgatgggtggccaggtagcaagagggcctatggagttggcaagtgaatttaaagtggtggcaccagagggcagatggggaggagcaggcactgtcatggactgtctatagaaatctaaaatgtataccctttttagcaatatgcagtgagtcataaaagaacacatatatatttaaattgtgtaattccacttctaaggattcatcccaaggggggaaaataatcaaagatgtaaccaaaggtttacaaacaagaactcatcattaatcttccttgttgttatttcaacgatattattattattactattattattattattattttgtctttttgcattttctagggccactcccacggcatagagaggttcccaggctaggggtcaaatcggagctacagctgccggcctacgccagagccacagcaacgcaggatctgagccacagcaatgcaggatctacaccacagctcatggtaacgctggatccttaacccaatgagtgaggccagggatcgaacctgtaacttcatggttcctagtcggattcattaaccactgagccacgacaggaactccaacattattaatgatgggagaaaactggaagtaacctaaatatccagcagaaagggtgtggccaaatacagcatggagtagccatcataaggaatcttacacaagcctccaaaattgtgtttctgaaattgggtttaaagtacgtttgcattttaaaaagcctgccagaaaatacagaaaaatgtctgtgatatgtctctggctgataggattttgcttagttttaattttggctttataattttctatagttatgaaaatgttcacaagaagatatatttcattttagcttctaaaataattataacacagaagtaatttgtgctttaaaaaaatattcaacacagaagtatataaagtaaaaattgaggagttcccatcgtggctcagtgattaacaaacccaactagtatccatgaggatatggatttgatccctggccttgctcagtgggttgaggatccagtgttgctgtgagctgtggtgtaggttgcagacacagcactctggcgttgctgtgactctggcgtaggccggcagctacagctccatttggacccttagcctgggaacctccatatgcctgagatacggccctaaaaagtcaaaagccaaaaaaatagtaaaaattgagtgtttctacttaccacccctgcccacatcttatgctaaaacccgttctccagagacaaacatcgtcaggtgggtctatatatttccagccctcctcctgtgtgtgtatgtccgtaaaacacacacacacacacacacacgcacacacacacacacgtatctaattagcattggtattagtttttcaaaagggaggtcatgctctaccttttaggcggcaaatagattatttaaacaaatctgttgacattttctatatcaacccataagatctcccatgttcttggaaaggctttgtaagacatcaacatctgggtaaaccagcatggtttttagggggttgtgtggatttttttcatattttttagggcacacctgcagcatatggaggttcccaggctaggggttgaatcagagctgtagctgccggcctacaccacagccacagcaacgccagatccttaacccactgagaaaggccagggattgaacctgcatcctcatggatgctggtcagatttatttctgctgagccacaacaggaactccctgaaccagaatgcttttaaccattccactttgcatggacatttagattgtttccatttaaaaatacaaattacaaggagttcccgtcgtggctcagtggtaacgaattggactaggaaccatgaggtttcgggttcgatccctggccttgctcggtgggttaaggatccagcattgatgtgagatatggtgtaggtcgcagacgtggctcggatcccacgttgctgtggctctggcgtaggccggcaacaacagctccgattcgacccctagcctgggaacctccatgtgccacaggagcagccctagaaaaggcaaaaagacaaaaaaataaaaaattaaaatgaaaaaataaaataaaaatacaaattacaagagacggctacaaggaaatccccaagtgtgtgcaaatgccatatatgtataaaatgtactagtgtctcctcgcgggaaagttgcctaaaagtgggttggctggacagagaggacaggctttgacattctcataggtagtagcaatgggcttctcaaaatgctgttccagtttacactcaccatagcaaatgacagtgcctcttcctctccacccttgccaataatgtgacaggtggatctttttctattttgtgtatctgacaagcaaaaaatgagaacaggagttcctgtcgtggtgcagtggagacaaatctgactaggaaccatgaaatttcgggttcaatccctggcctcactcagtaggtaaaggatccagggttgcagtgagctgtggggtaggtcgcagacacagtgcaaatttggccctgttgtggctgtggtgtaggccggcagctatagctccaattggacccctagcctgggaacctccttatgccgtgggtgaggccctaaaaaaaagagtgcaaaaaaaaaaaataagaacaaaaatgatcatcgtttaattctttatttgatcattggtgaaacttattttccttttatatttttattgactgattttatttctcctatgaatttaccggtcatagttttgcctgggtgtttttactccggttttagttttggttggttgtattttcttagagagctatagaaactcttcatctatttggaatagtaattcctcattaagtatttgtgctgcaaaaaattttccctgatctgttttatgcttttgtttgtggggtctttcacgagaaagcctttttagtttttacacctcagcttggttgtttttcttgattgtgtctgtaatctgcggccaacataggaaacacatttttactttagtgtttttttcctattttcttcaagtacgtccattgttttggtgtctgattttactttgcctggggtttgtttttgtgtggcaggaatataaacttatgtattttccaaatggagagccaatggttgtatatttgttgaattcaaatgcaactttatcaaacaccaaatcatcgatttatcacaactcttctctggtttattgatctaatgatcaattcctgttccacgctgttttaattattttagctttgtggattttggtgcctggtagagaacaaagcctccattattttcattcaaaatagtcccgtctattatctgccattgttgtagtattagactttaaaatcaatttactgattttcaaaagttattcctttggtgatgtggaatactttatacttcataaggtacatggattcatttgtggggaattgatgtctttgctattgtggccatttgtcaagttgtgtaatattttacccatgccaactttgcatattgtatgtgagtttattcccagggtttttaataggatgtttattgaagttgtcagtgtttccacaatttcatcgcctcagtgcttactgtttgcataaaaggaaacctactcacttttgcctattgctcttgtattcaatcattttagttaactcttgtgttaattttgagagtttttcagctgactgtctggggttttctttaatagactagccctttgtctgtaaagaataattttatcgaatttttcttaacactcacactctccccacccccacccccgctcatctcctttcattgggtcaaatctgtagaatacaataaaagtaagagtgggaaccttagcctttaagtcgattttgcctttaaatgtgaatgttgctatgtttcgggacattctctttatcaagttgcggatgtttccttagataattaacttaataaaagactggatgtttgctttcttcaaatcagaattgtgttgaatttatattgctattctgtttaattttgtttcaaaaaatttacatgcacaccttaaagataaccatgaccaaatagtcctcctgctgagagaaaatgttggccccaatgccacaggttacctcccgactcagataaactacaatgggagataaaatcagatttggcaaagcctgtggattcttgccataactctcagagcatgacttgggtgttttttccttttctaagtattttaatggtatttttgtgttacaataggaaatctaggacacagagagtgattcaatgaggggaacgcattctgggatgactctaggcctctggtttggggagagctctattgaagtaaagacaatgagaggaagcaagtttgcagggaactgtgaggaatttagatggggaatgttgggtttgaggtttctatagggcacgcaagcagagatgcactcaggaggaagaaggagcataaatctagaggcaaaaagagaggtcaggactggaaatagagatgcgagacaccagggtggcagtcagagagcacagtgtgggtcagaagacagtggaagaacacaagggacagagagggatctccaacttcactgggatgagggccttgttggccttgacctgagagatttccaggagttgagggtgggaaggagagggctcctgcacatgtcctgacatgaaacggtgcccagcatatgggtgcttggaagacattgttggacagatggatggatgatggatgatggatgaatggatggatggaagatgatggataaatggatgatggatggatggacagaaggacaaagagatggacagaaagacagtgatctgagagagcagagaaggcttcatgaaaggacaggaactgaactgtctcagtgggtggagacaatggtgtagggggtttccacatggaggcaccaggggtcaggaataatctagtgtccacaggcccaggaaggaagctgtctgcaggaaattgtggggaagaacctcagagtccttaaatgaggtcaggagtggtcaggagggtctgatcaggtaaggactcatgtccatcatcacatggtcacctaagggcatgtagctctcagcatctccatcaggacagtctcagaatgggggcggggtcacacactgggtgactcaaggcgtgggtcatgcctgcctcggacgtgggcctgggcatggggacacctccagaccatgggcccgcccagggctgcactggcctctggtgggctagctacccgtccaagcaacacaggacacagccctacctgctgcaaccctgtgcccgaaacgcccatctggttcctgctccagcccggccccagggaacaggactcaggtgctagcccaatggggttttgttcgagcctcagtcagcgtggtatttctccggcagcgagactcagttcaccgccttaggttaagtggttctcatgaatttcctagcagtcctgcactctgctatgccgggaaagtcacttttgtcgctgggggctgtttccccgtgcccttggagaatcaaggattgcccaactttctctgtgggggaggtggctggtcttggggtgaccagcaggaagggccccaaaagcaggagcagctgcctccagaatacaactgtcggctacagctcaaacaggaggcctggactggggtttaaccaccagggcggcacgaaggagcgaggctgggagggtgaggacatgggagcctgaggaggagctggagacttcagcaggcccccagctccgggcttcgggctctgagatgctcggacgcaaggtgagtgaccccacctgtggctgacctgacctcagggggacaaggctcagcctgagactctgtgtccccatcgcctgcacaggggattcccctgatggacactgagccaacgacctcccgtctctccccgacccccaggtcagcccaaggccgcccccacggtcaacctcttcccgccctcctctgaggagctcggcaccaacaaggccaccctggtgtgtctaataagtgacttctacccgaagggcgaattccagcacactggcggccgttactagtggatccgagctcggtaccaagcttgatgcatagcttgagtatcta

SEQ ID NO: 33 - DNA, 4614 bp, Sus scrofa
agatctttaaaccaccgagcaaggccagggatcgaacccgcatcctcatgaatcctagttgggttcgttaaccgctgaaccacaatgggaactcctgtctttcacatttaattcacaacctctccaggattctgggggtgggtggggaatcctaggtacccactgggaaagtaatccaaggggagaggctcacggactctagggatcggcggaggagggaaggtatctcccaggaaactggccaggacacattggtcctccgccctccccttcctcccactcctcctccagacaggactgtgcccaccccctgccacctttctggccagaactgtccatggcaggtgaccttcacatgagcccttcctccctgcctgccctagtgggaccctccatacctccccctggaccccgttgtcctttctttccagtgtggccctgagcataactgatgccatcatgggctgctgacccacccgggactgtgttgtgcagtgagtcacttctctgtcatcagggctttgtaattgatagatagtgtttcatcatcattaggaccgggtggcctctatgctctgttagtctccaaacactgatgaaaaccttcgttggcatagtcccagcttcctgttgcccatccataaatcttgacttagggatgcacatcctgtctccaagcaaccacccctcccctaggctaactataaaactgtcccaatggcccttgtgtggtgcagagttcatgcttccagatcatttctctgctagatccatatctcaccttgtaagtcatcctataataaactgatccattgattatttgcttctgttttttccatctcaaaacagcttctcagttcagttcgaattttttattccctccatccacccatactttcctcagcctggggaacccttgcccccagtcccatgcccttcctccctctctgcccagctcagcacctgcccaccctcacccttcctgtcactccctaggactggaccatccactggggccaggacactccagcagccttggcttcatgggctctgaaatccatggcccatctctattcctcactggatggcaggttcagagatgtgaaaggtctaggaggaagccaggaaggaaactgttgcatgaaaggccggcctgatggttcagtacttaaataatatgagctctgagctccccaggaaccaaagcatggagggagtatgtgcctcagaatctctctgagattcagcaaagcctttgctagagggaaaatagtggctcaaccttgagggccagcatcttgcaccacagttaaaagtgggtatttgttttacctgaggcctcagcattatgggaaccgggctctgacacaaacacaggtgcagcccggcagcctcagaacacagcaacgaccacaagctgggacagctgcccctgaacggggagtccaccatgcttctgtctcgggtaccaccaggtcaccatccctgggggaggtagttccatagcagtagtcccctgatttcgcccctcgggcgtgtagccaggcaagctcctgcctctggacccagggtggacccttgctccccactaccctgcacatgccagacagtcaagaccactcccacctctgtctgaggcccccttgggtgtcccagggcccccgagctgtcctctactcatggttcttccacctgggtacaaaagaggcgagggacacttttctcaggtttgcggctcagaaaggtaccttcctagggtttgtccactgggagtcacctcccttgcatctcaatgtcagtggggaaaactgggtcccatggggggattagtgccactgtgaggcccctgaagtctggggcctctagacactatgatgatgagggatgtggtgaaaaaccccaccccagcccttcttgccgggaccctgggctgtggctcccccattgcacttggggtcagaggggtggatggtggctatggtcaggcatgtttcccatgagctgggggcaccctgggtgactttctcctgtgaatcctgaattagcagctataacaaattgcccaaactcttaggcttaaaacaacacacatttattcctctgggtcccagggtcagaagtccaaaatgagtcctataggctaaatttgaggtgtctctgggttgagctcctcctggaagccttttccagcctctagagtcccaagtccttggctctgggcccctccctcaagcttcaaagccacagaagcttctaatctctctcccttcccctctgacctctgctcccatcctcataccctgtcccctcactctgaccctcctgcctccctctttcccttataaagaccctgcatggggccacggagataatccagggtaatcgcccctcttccagcccttaactccatcccatctgcaaaatccctgtcaccccataatggacctactgatggtctgggggttaggacgtggacaacttggggccttattcatctgatcacaactccagttcccagacccccagacccccgggcattagggaaacttctcccagttcctctccctctgtgtcctgcccagtctccaggatgggccactcccgagggcccttcagctcaggctccccctcctttctccctggcctcttgtggccccatctcctcctccgctcacagggagagaactttgatttcagctttggctctggggctttgcttccttctggccattggctgaagggcgggtttctccaggtcttacctgtcagtcatcaaaccgcccttggaggaagaccctaatatgatccttaccctacagatggagactcgaggcccagagatcctgagtgacctgctcacattcacagcagggactgaaccccagtcacctacccaactccagggctcagcgcttttttttttttttttctttttgccttttcgagggccgctcccgcaacatatggagatttccaggctaggggtctaattggagcagtcgacactggcctaagccaaagccacagcaacaagggcaagccgcttctgcagcctataccacagctcacggcaatgccggatccttaacccactgagcaaagccagggattgaacctgcaacctcatgtttcctagtcaaatttgttaaccactgacccatgacgggaactcccagggctcagctcttgactccaggttcgcagctgccctcaaagcaatgcaaccctggctggccccgcctcatgcatccggcctcctccccaaagagctctgagcccacctgggcctaggtcctcctccctgggactcatggcctaagggtacagagttactggggctgatgaagggaccaatggggacaggggcctcaaatcaaagtggctgtctctctcatgtcccttcctctcctcagggtccaaaatcagggtcagggccccagggcaggggctgagagggcctctttctgaaggccctgtctcagtgcaggttatgggggtctgggggagggtcaatgcagggctcacccttcagtgccccaaagcctagagagtgagtgcctgccagtggcttcccaggcccaatcccttgactgcctgggaatgctcaaatgcaggaactgtcacaacaccttcagtcaggggctgctctgggaggaaaaacactcagaattgggggttcagggaaggcccagtgccaagcatagcaggagctcaggtggctgcagatggtgtgaaccccaggagcaggatggccggcactccccccagaccctccagagccccaggttggctgccctcttcactgccgacacccctgggtccacttctgccctttcccacctaaaacctttagggctcccactttctcccaaatgtgagacatcaccacggctcccagggagtgtccagaagggcatctggctgagaggtcctgacatctgggagcctcaggccccacaatggacagacgccctgccaggatgctgctgcagggctgttagctaggcggggtggagatggggtactttgcctctcagaggccccggccccaccatgaaacctcagtgacaccccatttccctgagttcacatacctgtatcctactccagtcaccttccccacgaacccctgggagcccaggatgatgctggggctggagccacgaccagcccacgagtgatccagctctgccaatcagcagtcatttcccaagtgttccagccctgccaggtcccactacagcagtaatggaggccccagacaccagtccagcagttagagggctggactagcaccagctttcaagcctcagcatctcaaggtgaatggccagtgcccctccccgtggccatcacaggatcgcagatatgaccctaggggaagaaatatcctgggagtaaggaagtgcccatactcaaggatggcccctctgtgacctaacctgtccctgaggattgtacttccaggcgttaaaacagtagaacgcctgcctgtgaacccccgccaagggactgcttggggaggccccctaaaccagaacacaggcactccagcaggacctctgaactctgaccaccctcagcaagtggcaccccccgcagcttccaaggcac

SEQ ID NO: 34 - DNA, 1151 bp, Sus scrofa
aacaagatgctaccccaccaacaaaattcaccggagaagacaaggacagggggttcctggggtcctgacagggtcaccaaagagggttctggggcagcagcaactccagccgcctcagaacagagcctggaagctgtaccctcagagcagaggcggagagagaaagggcctcttggtgggtcagcaggagcagaggctcagaggtgggggttgcagcccccccttcaacaggccaacacagtgaagcagctgacccctccaccttggagaccccagactcctgtctcccacgccaccttggtttttaaggtaatttttattttatatcagagtatggttgacttacaatgttgtgttggtttcaggtgtacagcagagtgattcacttctacatagactcatatctattctttctcagattcttttcccatataggttattacagaatattgagtagatccctgctgattacccatttttataattgtatatgttaatcccaaactcctaatttatccctccccagactatgattctttatatctctatctgtttcctaatctgtctcctctaagtcaccctaggagagcagaggggtcacgtctgtcctgtcctggcccagccacctctctccacccaggaatcccttgcatttggtgccaagggcccggccccgccctaaagagaaaggagaacgggatgtggacaggacaccgggcagagagggacaagcagaggatgccagggtagggaggtctccagggtggatggtggtctgtccgcaggcaggatgaggcaggaagggtgtggatgtactcggtgaggctggcgcatggcctggagtgtcctgagccctgggaggcctcagccctggatcagatctgtgattccaaagggccactgcatccagagaccgttgagtggcccattgtcctgaaccatttatagaacacaggacaagcggtacctgactaagctgctcacagattccatgaggctgatgccagggttgtcaccccatctcacaggcagggaaactgatgcatatactgcagagccaggcagaggccctcccagtgccccctcccagcctgtggcccccctccagtggctggacactgaggccacactggggcaccctgtggagatct

SEQ ID NO: 35 - DNA, 630 bp, Sus scrofa
agatctggccaggccagagaagcccatgtggtgacctccctccatcactccacgccctgacctgccagggagcagaaagtaggcccagggtggacccggtggccacctgccaccccatggctgggagaagggagggcctgggcaaagggcctgggaagcctgtggtgggaccccagaccccagggtggacagggagggtcccacacccacagccatttgcttccctctgtgggttcagtgtcctcatctcatctgtggggagggggctgataatgaatctcccccattggggtgggcttggggattaaagggccagtgtctgtgatatgcctggaccatagtgaccctcaccctccccagccattgctgtcaccttccgggctcttgcccaggcctgcctgacatgctgtgtgaccctgggcaagatgatccccctttctgggccccagccttcctctctgctccggaagtgcttcctggggaaacctgtgggctggatcctataggaaacctgtccaattcctggatgcacagaggggcagggaggccctgggcctggaggggcagggaggctcgaggtgggagcagggtaggggccagtccagggcaaggaggtgggtgggtagggtg

SEQ ID NO: 36 - DNA, 947 bp, Sus scrofa
gatctgtgttccatctcagagctatcttagcagagaggtgcaggggcctccagggccaccaaagtccaggctcagccagaggcaatggggtatcgatgagctacaggacacaggcgtcagcccagtgtcagggagaatcaccttgtttgttttctgagttcctcttaaaatagagttaattggtcttggccttacggtttacaataacaactgcaccctgtaaacaacgtgaagagtacagaacaacaaatgggggaaaacatatttcacctgaaagagccaccgctcatattttgatggatttccttctagtttaatcctgttttaattgtaaactgttaaaacaaacataaataaagaaaatgcatctgtaaagtttaaaagtcatatctatggtgatggttgcaaaacactgtgaatgttcactttgaaatcgtgaactctacgtgatatgcatgtcccgttaattaacctcacaggctcagaatgtggttcattatttctttaattttcctttaattttatgtcctctgtgtgtgcccttaaaccaactacttttcagctctgcctgtttttgaccttcacatagatgacatttgtgagtgttttctttctcaacactgggtctgatacccacccacgctgtctgctgtcactgcggacgtggagggccaccacccagctatggccccagccaggccaacactggatgaatctgcccccagagcagggccaccaacactggaggtgcagagagggtttcttcagggccatcattatccaaggcattgtttctactgtaagctttcaaaatgcttcccctgattattaaaagaaataataagatggggggaaagtacaagaagggaagtttccagcccagcctgaagatcgtgctggttgtatctggagcctgtcttcctgacaggcctctattcccagagtta

SEQ ID NO: 37 - DNA, 2067 bp, Sus scrofa
ggatcctagggaagggagggcgggggcctggacaaagggggcctaaaggacattctcacctatcccactggacccctgctgtgctctgagggagggagcagagagggggtctgaggccttttcccagctcctctgagtccctcctccgagcacctggacggaagcccctcctcagggagtcctcagacccctcccctccagccaggttggcctgtgtggagtccccagtaagaatagaatgctcagggcttcgagctgagccctggctacttgggggggtgctggggattgggggtgctgggcggggagctggggtgtcactagatgccagtaggctgtgggctcgggtctggggggtctgcacatgtgcagctgtgggaaggccctattggtggtaccctcagacacatatggcccctcaatttctgagaccagagaccccagtctggccttcccagaacagctgcccctggtgggggagatgtaggggggccttcagcccaggacccccaacggcagggcctgaggcccccatccccttgtcctgggcccagagcctcagctatcaggcctatcagagatcctggctgcccagctcaggttccccaggagccagagggaggccaggggttactaggaaatccggaaagggtctttgaggctgggccccaccctctcagctttcacaggagaaacagaggcccacagggggcaaaggacttgccagactcacaatgagcccagcagctggactcaaggcccagtgttcggccccacaacagcactcacgtgcccttgatcgtgaggggccccctctcagccaggcattcagacctgtgacctgcatctaagattcagcatcagccattctgagctgaagagccctcagggtctgcagtcaaggccacagggccagacctccaacggccagacatcccagccagattcctttctggtcaatgggccccagtctggcttggctcctgcaggcccagtgccgccttcttcccctgggcctgtggagtccagcctttcagtttcccacccacatcctcagccacaatccaggctcagaggcaatgtccgtgggcagcccctgtgtgacccctctgtgggtgatcctcagtcctacccttagcagacagcgcatgaggggccctcttgaacctgagggatactccatgtcggaggggagaagctggccttccccacccccacttccaggccttggggagcagagaaagaccccagacctgggtcccttctaacaggccaggccccagcccagctctccaccagccccaggggcctcgggtccacgcctggggactggagggtgggcctgtcaggcgctgacccagaggcaggacagccaagttcaggatcccagccaggtggtccccgtgcaccatgcaggggtgtcacccacacaggggtgttgccaccctcacctgactgtcctcatgggccacatggaggtatcctgggttcattactggtcaacatacccgtgtccctgcagtgccccctctggcgcacgcgtgcacgcgcacacgcacacactcatacagaggctccagccaacagtgccctctagtaggcactgctgtcacttctctaaaaggtcgcaatcatacttgtaaagacccaagattgttcagaaatcccagatggagaagtctggaaagatctttttctcctttcacgggctggggaaatgtgacctggccaaggtcacacagcaagtggtggaaccctggcccctgattccagctcattccagttcccaaggccctgccagagcccagaggctgggccctctggggcagaggagctggggtcctcccccctacacagagcacacagccccgcaagagagaagagacaccttggggagaggaatctccagaccagagatcccagtatgggtctcctctatgctgacgggatgggatgtcaagaggggagggggctgggctttagggaaacacacaaaaatcgctgagaacactgacaggtgcgacacacccacccctaatgctaacctgtggcccattactcagatct

SEQ ID NO: 38 - DNA, 4898 bp, Sus scrofa
gatcttctcctaagaccaaggaaaactggtcataccaggtccacttgtcccctgtggccattgtccctccttccccagaagaaacaagcactttccactccacaagtagctcctgatcagcttggaagcccggtgctgctctgggccctggggacacggcaggggcatcagagaccaaatcctggaacaaagttccagtgggtgaggcaggccggacaagcaacacgttataccataatatgaggcaaaatataatgtgagttctttatgaaaggaaggggttgcaggtgcaactgttggcttaggtggatggtcacccctgaatggaggagggggttcccagggcatgtgcctggggagaagggctcctggcaggagggacagcaagtgcaagggccctgtgatcaaatgtgcctggcaagttgcaggaacagctagaaggccagcaaggttggaaccaaggaaggggtcaggggaggggcagggccctcagggccttgcccagcagcctgagcatctggagatttgtccaaagtttcaaatgtacctgggcaacctcatgcccatataccattcctaacttctgcacttaacatctctaggactgggacccagccagtcaagcggggggacccagagagctccggtgtgaacaccgaggtgctggtgggtctgcgtgtgtggacatagggcagtcccggtccttccttcactaacacggcccgggaagccctgtgcctccctggtgcgcgggtcggcgcttccggagggtacaggcccacctggagcccgggcacagtgcatgcaagtcgggttcacggcaacctgagctggctctgcagggcagtgggactcacagccaggggtacagggcagaccggtcctgcctctgcgcccctccctggcctgtggcccctggacgtgatccccaacagttagcatgccccgccggtgctgagaacctggacgaggtccgcaggcgtcactgggcggtcactgagcccgccccaggccccctctgccccttcctggggtgaccgtggactcctggatgaccctggaccctagacttcccagggtgtctcgcggaggttcctcagccaggatctctgcgtctcctccttccatagaggggacggcgcccccttgtggccaaggaggggacggtgggtcccggagctggggcggagaacacagggagcccctcccagaccccgctctgggcagaacctgggaagggatgtggccatcgggggatccctccaggccatctcctcagatgggggctggtcgactagcttctgagtcctccaaggaaccgggtccttctagtcatgactctgcccagatgaagaaggagagcacttctctccatcaggaggatctgagcttctcttaattagaatcagctccttggcttctaccccttaaaaaaaggtacagaaactttgcaccttgatccagtatcaggggaatttatcaatcaatgtgggagaaattggcatctttaccacactgaatctttcaatccatgaatatcctctctctcttccatgcataggttttaataattctcaatggagtttaatgtaagttttcctcatagacaattgcctttggacatctctttagactcatctctagtaaactgatattcttaatgcaattataaaatgtatcctgcttaatgttattttctattcatttgctgttatatagagatacaatgagtttccacatttgaaactggatctggtaaattggctaccctttttttatagattctattaatttttatacattctgtgggacttgctacatacttaatcatgtcacctgtgaagaatgacaatttggttgctaccctcccaattcttatatgtctcatttctttccctctgctggtactctggcagcagcagggaagataatgggcctccttatcttgtcacaaaaggatgtttttaaagatttcgttataaaacataacgctttctggttttctttaaagattctctcaccagcttaagaaaattttcttatactctgtatgataaatgggtttttgacaatcatttgttgcattttacctagtgttttctctgcatctttatatgctttttctcctttaatcctgaaaattgtttcgatttttctaacattgaaccaatcttacattcctggaatggatggaccagactagtccacatgtttattctgcccaatggctagattttgtgttcaatattttgttcagaatgtttgcatctatattcttgagtgagacagagctgcccttgttaggtttcacaaccgaggttgtgttagcttcataaaatgagacgtttattctctaaaagaattgtttcgcttctctggatgaatttgtgtaaggttagaattgcttaccagtgaagatctcggggccagttcttctttaggggaagattttcaacaattaagctcaatgcctttagaagaactgagagtttctattatttcttgagttaaatatatgtatttaattagactttctaggaatagtctcatttcatctcaaataattgacatatgctattaaagcagattctcatgaaccattgtaggtattccaggtctagaaaaatgttcccctttgcatccctaatgtgtttaattttcaccttctttcttttgttcttgagaaattcaccaaatcattttcaatttcagtcatatcccaaagcaaccaactctctaccttcttgttttatcatccctgctggatttttgttatctacttcttcagtatttgttcttccctttcttctattcctcattccatttttcccttgttttctaactttctgagatatatgcttagttccttcatttgaagcctttttattttctttttttttttttggtctttttgtcttttgttgttgttgttgtgctatttcttgggccgctcccgcggcatatggaggttcccaggctaggagtcgaatcggagctgtagccaccggcctacgccagagccacagcaatgcgggatccgagccgcgtctgcaacctacaccacagctcatggcaacgccggatcgttaacccactgagcaagggcaggaaccgaacccgcaacctcatggttcctagtcggattcgtaaccactgtgccacaacaggaactccgcctttttattttctataaaaatttctatgtacattttaaggttataggtttccttctatgtaccccattggctgtatcctcagggttctgtggagtgatttcattattgttcaagttcaatatgtcttctgattttccaatttgaatacctctctaaatcagtaggtgaatatttctttttctttttcttttcttttcttctttttttttttctttcagccaggtccatggcatgcagaaattcccaggccaggaatcaaactctcaccatggcagtgacaatgtcggatcctttacccactaggccaccagggaactctgggagcatatgtttttatttcccgacatctgaggatgcctagtatgtcttcattattgatttctagtttgccactgatttctagtattttgctcatagagtgtatgctcaatggttttggtcatttgaaatgtatttagtcctgctttatgacccagtatgtggtcagttttgtcaatgttccttttctgcttgaagagaacctacatgctgtaactctgggtgcatgttctgtatataagtctataggctgagccgggggagccttctaatctgccgttatcttcttcgagttattctaggtactatttcttagccataaacctttaaattctgatatcaatataatgaccccagcccgcttagggtcggcacttcatgttatctttttccatccatttaatccctccccactgttttggccacacccgtgggatatgggagttcctgggccaaggatcagatctgagccgcagctgccacctatgccacagcagcagcaatgatggatctttaacccactgcaccacactggggattgaacccaagcctcagcagcaacccaagctactgcagagacaacaccagatccttaacctgctgtgccatagcgggaatttccatccatttactttcaagccagctgaataacctagcccaccatgcctggacatgggtgctctgcttcaaatgattttgttcagtcagcatccatctctgaaatgtgtgccaagcatttatatgcatgcaagagtcatgttggcacttctatcatttccaacagttcagtagcctttgtatcatgacatttcttggccttttctctacaatatttgaggctgagcagactggccgtgcccctgtccatgcttccagagcctgtgtgcagacttctgctctagacagagacagctaaccatcctgcagtgcccagaaaacccaactcaaagaccctcaagtaaggaaggatttattggctcacgtaatctggaatccaggcatggggtattcagggccacctgaaccagaggccctggccctgttctctaagcttcttcctgccctgccctcgttctggaagtgaccctgaaggacagcaatgaagggcagctcccccagggacagatgactgagaggtccatttcaagtccaacttggcctagattgagaggcagcaagaaatatggacctacagtgagtcacaggatttaccagtggtttggctgggttgtcagtgttacaggctaaacatttgggtccctccaaaattaacatgttgccactctaaccaccaaaatcatggtatttgggggtggggcccttggaggtaattaggtttagaaagaatgaagagggggcccttgtgatgggactagtgcctttatagagagagaagagagaggg

SEQ ID NO: 39 - DNA, 1913 bp, Sus scrofa
cacctcatccccaaccacctggatggtggcaagtggcaggctgagaggctgcatatgagctcatcaagagggtccccaccccacagaggctgacccagctgccactgccacctagtggctgatcggccaagagcaggagccccaggggcagctccattccctggggcggccagggaaccacctggtggtaggacaattccattgcacctcatccatcaggaaaaggtttgccttccctggcagtaatgcatcttcccataacatggtccctggcctcttggaatggcttggccaccgtcatggcctcacccacaaagccttgtgtctcagcaaggaacttattccacagcaaaggacttgcagcctggaatgaactggtctgactacataccccattgcccagaagtaggtggtctattgcaaagtggagtggcttacccaagactcagttgtgcccaagttgagagatagcatcctaaaatatgggcttatgtctcactggctgaggtttattctttgaatcaaagacaattatatggtgtggtccccccagagatagaatacatgagtctgggaatcaagggatagaagtaagaagagattttgtcaccattaatcccaataactcgcccaaagaatatttgctttctgtcctggcagctctgctgctttggcaataacttcctagaatataatgtctccaccaggggactccacaacggttccattgatttgaagccaatgggcagaggaggggctgccttactggtcggactggtcagccctgattactaaggagaaatcaggcaacttcaacaaaactaaggcaggggggactttgtctagaacccaaagcactaagcatcttagtactttttagttctcagagcctccaagaacaaagatttagcccctcagcaccaccaggtaaagaacaggtaaatccagctgaggacaagagaaatattgaatggatagaggaagaaagaaattatagatatcaactatggcctcatgactagagtctccagattaagcggaataaaaatacagatgattagatctgaacatcaggccaaacaacgaacaacagtttaagtgcgacctaggcaatatttgggacatacttatactaaaattttttcgctatttgagcatcctgtattttatctggcaactttattcatccctagcgaaaaaggaactgtggtaacttagtgtatttttactttgctcattattgtgtatatacctacttgtatttatcaatcatatttactctgttctcagtattactttatatagcagttggtggtgatggttagcaacatattcagtggaactgtgactgaatttgaggagaaattaacagagttggctgtggctacaataacccttcgggacatgtgtcccctcattttggggagatggttagatctctgggtaaatgttagggcatctgagccagaaaccaagattttgccagctggtgcaatgtcagattttaccagcagagggtgccagaggaatgcggcaaaacccgagtgccagaaagcacctccctgttttccagcttttcttcctttttatttattttatttacggcccaggagtccgtaatagcgctgaggatggcccaggctcttctcagcagccctgactgactagttcagcaatgcgctcaggccccatctggccaccgggcagcctcttctgtggtagctccagcctcagccagtgcaaaaggctaccctacactggcgccacttctacaatcagcactggccacaccctccacgccatccggcacggagccaggtgatctgccggccagattgcagttcgtgctgcctgagtccaggtgattacactggctgcatcttttctttctggaccattcattccattttttt

SEQ ID NO: 40 - DNA, 394 bp, Sus scrofa
ctctgcactcactaccgccggacgcgcactgccgtgctgcccatggaccacgctggggaggggtgagcggacagcacgttaggaagtgtgtgtgtgcgcgtgggtgcaagtcgagccaaggccaagatccaggggctgggccctgtgcccagaggagaatggcaggtggagtgtagctggattgaaaggtggcctgaagggtggggcatcctgtttggaggctcactctcagccccagggtctctggttcctgccggggtggggggcgcaaggtgcctaccacaccctgctagcccctcgtccagtcccgggcctgcctcttcaccacggaagaggataagccaggctgcaggcttcatgtgcgccgtggagaacccagttcggcccttggagg

SEQ ID NO: 41 - DNA, 333 bp, Sus scrofa
ggctgaagtctgaggcctggcagatgagcttggacgtgcgctggggagtactggagaaggactcccgggtggggacgaagatgttcaagacggggggctgctcctctacgactgcaggcaggaacggggcgtcactgtgccggcggcacccggccccgcccccgccacagccacagggggagcccagctcacctggcccagagatggacacggacttggtgccactggggtgctggacctcgcacaccaggaaggcctctgggtcctgggggatgctcacagagggtaggagcacccgggaggaggccaagtacttgccgcctctcaggacgg

SEQ ID NO: 42 - DNA, 399 bp, Sus scrofa
gaagtgaagccagccagttcctcctgggcaggtggccaaaattacagttgacccctcctggtctggctgaaccttgccccatatggtgacagccatctggccagggcccaggtctccctctgaagcctttgggaggagagggagagtggctggcccgatcacagatgcggaaggggctgactcctcaaccggggtgcagactctgcagggtgggtctgggcccaacacacccaaagcacgcccaggaaggaaaggcagcttggtatcactgcccagagctaggagaggcaccgggaaaatgatctgtccaagacccgttcttgcttctaaactccgagggggtcagatgaagtggttttgtttcttggcctgaagcatcgtgttccctgcaagaagcgg

SEQ ID NO: 43 - DNA, 23 bp, Sus scrofa
gtctttggtttttgctgagggtt